(19) United States
(12) Patent Application Publication
(10) Pub. No.: US 2025/0320227 A1
(43) Pub. Date: Oct. 16, 2025

(54) MACROCYCLIC OREXIN RECEPTOR AGONISTS AND USES THEREOF

(71) Applicant: Jazz Pharmaceuticals Ireland Limited, Dublin (IE)

(72) Inventors: Claudia BEATO, Castel d'Azzano (IT); Prafulkumar CHOVATIA, Abingdon (GB); Davide MARINELLI, Verona (IT); Gilles OUVRY, Abingdon (GB); Ricky Michael CAIN, Abingdon (GB); Diego FIORUCCI, Verona (IT)

(21) Appl. No.: 18/869,941

(22) PCT Filed: Jun. 1, 2023
(86) PCT No.: PCT/EP2023/064735

§ 371 Date: Nov. 27, 2024

Related U.S. Application Data
(60) Provisional application No. 63/347,708, filed on Jun. 1, 2022.

(51) Int. Cl. C07D 498/20; A61K 31/439; A61K 31/537; A61K 31/5386; A61K 31/547; C07D 498/22; C07D 513/20

(52) U.S. Cl. CPC C07D 498/20; A61K 31/439; A61K 31/537; A61K 31/5386; A61K 31/547; C07D 498/22; C07D 513/20

(57) ABSTRACT

Provided herein are compounds of Formula (I), (I), or pharmaceutically acceptable salt thereof, wherein m, n, p, A₁, A₂, A₃, A₄, L, R₁, R₂, R₃, R₄, Formula (II), (II), V, X, Y and Z are defined herein. Also provided herein are pharmaceutical compositions comprising a compound of Formula (I) or pharmaceutically acceptable salt thereof, and methods of using a compound of Formula (I) or pharmaceutically acceptable salt thereof, e.g., in the treatment of a disease or disorder that is treatable by administration of an Orexin agonist.

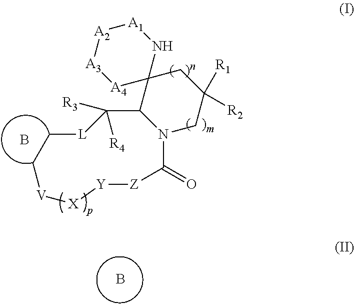

### CROSS-REFERENCE TO RELATED APPLICATIONS

This application claims the benefit of priority to U.S. Provisional Application No. 63/347,708, filed Jun. 1, 2022, which is incorporated herein by reference in its entirety.

### BACKGROUND

Orexin is a neuropeptide specifically produced in particular neurons located sparsely in the lateral hypothalamus and its surrounding area. Orexin consists of two subtypes, orexin A and orexin B. Both orexin A (OX-A) and orexin B (OX-B) are endogenous ligands of the orexin receptors, which are mainly present in the brain. Two orexin receptors have been cloned and characterized in mammals. They belong to the super family of G-protein coupled receptors: the orexin-1 receptor (OX or OX1R) is partially selective for OX-A and the orexin-2 receptor (OX2 or OX2R) is capable of binding OX-A as well as OX-B with similar affinity. The physiological actions in which orexins are presumed to participate are thought to be expressed via one or both of OX1 receptor and OX2 receptor as the two subtypes of orexin receptors.

Orexins regulate states of sleep and wakefulness making the orexin system a target for potential therapeutic approaches to treat sleep disorders. Orexins are found to stimulate food consumption in rats suggesting a physiological role for these peptides as mediators in the central feedback mechan ism that regulates feeding behavior. Orexins have also been indicated as playing a role in arousal, emotion, energy homeostasis, reward, learning and memory.

There is a need for compounds that modulate orexin receptors, as well as compositions and methods for treating a disease or disorder that is treatable by administration of an Orexin agonist.

### SUMMARY

The present disclosure is directed to compounds that are agonists of the orexin-2 receptor as well as pharmaceutical compositions thereof and uses thereof in treating a disease or disorder that is treatable by administration of an Orexin agonist.

In one aspect, the present disclosure provides a compound of Formula (I):

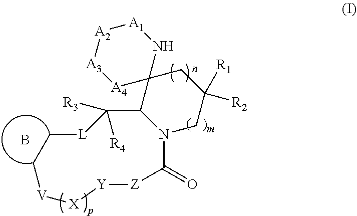

or a pharmaceutically acceptable salt or stereoisomer thereof,
wherein:
L is a linker selected from the group consisting of aryl, heteroaryl, -carbocyclyl-O—, and -heterocyclyl-O—, wherein-carbocyclyl-O— and -heterocyclyl-O— have the following orientation:

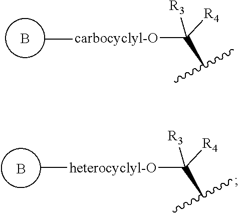

A₁ is —C(O)—, —S(O)₂—, or —C(H)(CF₃)—;
A₂ and A₃ are each independently a bond, —O—, —CR₅R₆—, —NR₇—, or —S—; or A₂ and A₃ together with an optionally substituted carbon atom form a cyclopropyl ring having the structure:

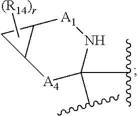

A₄ is a bond, —O—, —CR₅R₆—, —NR₇—, —S—, —(CR₅R₆)₂—, —CR₅R₆—O—, —CR₅R₆—S—, —CR₅R₆—N(R₇)—, —O—CR₅R₆—, —S—CR₅R₆—, or —N(R₇)—CR₅R₆—, with the proviso that the ring that includes A₂, A₃ and A₄ does not contain —O—O—, —O—NR₇— or —NR₇—NR₇—;
B is phenyl, 5- or 6-membered heteroaryl, cycloalkyl, or heterocyclyl, each of which is optionally substituted;
V and Z are each independently —O—, —CR₈R₉—, or —NR₁₀—;
X is —O—, —CR₁₁R₁₂—, or —NR₁₃—;
Y is a bond, —O—, —CR₈R₉—, or —NR₁₀—;
R₁, R₂, R₃, R₄, R₅, R₆, R₈, R₉, R₁₁, R₁₂ and R₁₄ are each independently hydrogen, halogen, alkyl, cycloalkyl, or heterocyclyl; and/or R₁ and R₂ together with the atom to which they are attached form a carbocycle or heterocycle; and/or R₃ and R₄ together with the atom to which they are attached form a carbocycle or heterocycle; and/or R₅ and R₆ together with the atom to which they are attached form a carbocycle or heterocycle; and/or R₈ and R₉ together with the atom to which they are attached form a carbocycle or heterocycle; and/or R₁₁ and R₁₂ together with the atom to which they are attached form a carbocycle or heterocycle;

R₇, R₁₀, and R₁₃ are each independently hydrogen, alkyl, cycloalkyl, heterocyclyl, aryl, heteroaryl, —(C═O) alkyl, —(C═O) cycloalkyl, —(C═O) heterocyclyl, —(C═O)—O-alkyl, —(C═O)—O-cycloalkyl, —(C═O)—O-heterocyclyl, —(C═O)—O-heteroaryl, —S(O)₂-alkyl, —S(O)₂-cycloalkyl, or —S(O)₂-heterocyclyl; and m, n, p, and r are each independently 0, 1, or 2.

In some embodiments, the present disclosure provides a compound of Formula (IA):

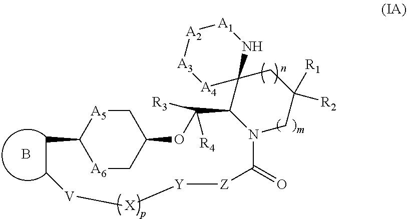

or a pharmaceutically acceptable salt thereof,
wherein:
A₁ is —C(O)—, —S(O)₂—, or —C(H)(CF₃)—;
A₂ and A₃ are each independently a bond, —O—, —CR₅R₆—, —NR₇—, or —S—; or A₂ and A₃ together with an optionally substituted carbon atom form a cyclopropyl ring having the structure:

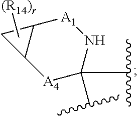

A₄ is a bond, —O—, —CR₅R₆—, —NR₇—, —S—, —(CR₅R₆)₂—, —CR₅R₆—O—, —CR₅R₆—S—, —CR₅R₆—N(R₇)—, —O—CR₅R₆—, —S—CR₅R₆—, or —N(R₇)—CR₅R₆—, with the proviso that the ring that includes A₂, A₃ and A₄ does not contain —O—O—, —O—NR₇— or —NR₇—NR₇—;
A₅ and A₆ are each independently —O— or —CH₂—;
\textcircled{B} is phenyl, 5- or 6-membered heteroaryl, cycloalkyl, or heterocyclyl, each of which is optionally substituted;
V and Z are each independently —O—, —CR₈R₉—, or —NR₁₀—; X is —O—, —CR₁₁R₁₂—, or —NR₁₃—;
Y is a bond, —O—, —CR₈R₉—, or —NR₁₀—;
R₁, R₂, R₃, R₄, R₅, R₆, R₈, R₉, R₁₁, R₁₂ and R₁₄ are each independently hydrogen, halogen, alkyl, cycloalkyl, or heterocyclyl; and/or R₁ and R₂ together with the atom to which they are attached form a carbocycle or heterocycle; and/or R₃ and R₄ together with the atom to which they are attached form a carbocycle or heterocycle; and/or R₅ and R₆ together with the atom to which they are attached form a carbocycle or heterocycle; and/or R₈ and Ry together with the atom to which they are attached form a carbocycle or heterocycle; and/or R₁₁ and R₁₂ together with the atom to which they are attached form a carbocycle or heterocycle;
R₇, R₁₀, and R₁₃ are each independently hydrogen, alkyl, cycloalkyl, heterocyclyl, aryl, heteroaryl, —(C═O) alkyl, —(C═O) cycloalkyl, —(C═O) heterocyclyl, —(C—O)—O-alkyl, —(C═O)—O-cycloalkyl, —(C═O)—O-heterocyclyl, (C═O)—O-heteroaryl, —S(O), alkyl, —S(O)₂-cycloalkyl, or —S(O)₂-heterocyclyl; and
m, n, p, and r are each independently 0, 1, or 2.

In some embodiments, the present disclosure provides a compound of Formula (IB):

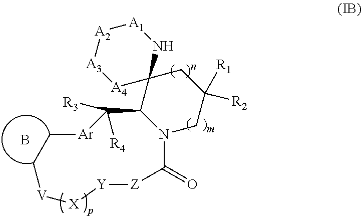

or a pharmaceutically acceptable salt thereof,
wherein:
A₁ is —C(O)—, —S(O)₂—, or —C(H)(CF₃)—;
A₂ and A₃ are each independently a bond, —O—, —CR₅R₆—, —NR₇—, or —S—; or A₂ and A₃ together with an optionally substituted carbon atom form a cyclopropyl ring having the structure:

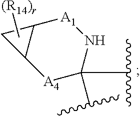

A₄ is a bond, —O—, —CR₅R₆—, —NR₇—, —S—, —(CR₅R₆)₂—, —CR₅R₆—O—, —CR₅R₆—S—, —CR₅R₆—N(R₇)—, —O—CR₅R₆—, —S—CR₅R₆—, or —N(R₇)—CR₅R₆—, with the proviso that the ring that includes A₂, A₃ and A₄ does not contain —O—O—, O—NR₇— or —NR₇—NR₇—;
Ar is an aryl or heteroaryl linker;
\textcircled{B} is phenyl, 5- or 6-membered heteroaryl, cycloalkyl, or heterocyclyl, each of which is optionally substituted;
V and Z are each independently —O—, —CR₈R₉—, or —NR₁₀—;
X is —O—, —CR₁₁R₁₂—, or —NR₁₃—;
Y is a bond, —O—, —CR₈R₉—, or —NR₁₀—;
R₁, R₂, R₃, R₄, R₅, R₆, R₈, R₉, R₁₁, R₁₂ and R₁₄ are each independently hydrogen, halogen, alkyl, cycloalkyl, or heterocyclyl; and/or R₁ and R₂ together with the atom to which they are attached form a carbocycle or heterocycle; and/or R₃ and R₄ together with the atom to which they are attached form a carbocycle or heterocycle; and/or R₅ and R₆ together with the atom to which they are attached form a carbocycle or heterocycle; and/or R₈ and R₉ together with the atom to which they are attached form a carbocycle or heterocycle; and/or R₁₁ and R₁₂ together with the atom to which they are attached form a carbocycle or heterocycle;
R₇, R₁₀, and R₁₃ are each independently hydrogen, alkyl, cycloalkyl, heterocyclyl, aryl, heteroaryl, —(C═O) alkyl, —(C═O) cycloalkyl, —(C—O) heterocyclyl, —(C═O)—O-alkyl, —(C═O)—O-cycloalkyl, —(C═O)—O-heterocyclyl, —(C═O)—O-heteroaryl, —S(O)₂-alkyl, —S(O)₂-cycloalkyl, or —S(O)₂-heterocyclyl; and
m, n, p, and r are each independently 0, 1, or 2.

In some embodiments, the present disclosure provides a compound of Formula (IC):

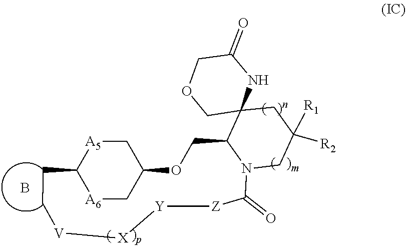

or a pharmaceutically acceptable salt thereof, wherein m, n, p, A₅, A₆, R₁, R₂, \textcircled{B}, V, X, Y and Z are as defined herein.

In some embodiments, the present disclosure provides a compound of Formula (ID):

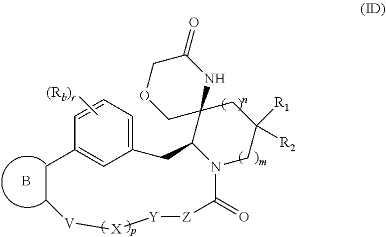

or a pharmaceutically acceptable salt thereof, wherein m, n, p, r, A₅, A₆, R₁, R₂, R_{b}, \textcircled{B}, V, X, Y and Z are defined herein.

In some embodiments, A₁ is —C(O)— or —S(O)₂—. In some embodiments, A₁ is —C(O)—. In some embodiments, A₁ is —S(O)₂—.

In some embodiments, A₂ is —O—, —NR₇—, or —CR₅R₆—. In some embodiments, A₂ is a bond, —O—, or —CR₅R₆—. In some embodiments, A₂ is —CR₅R₆—. In some embodiments, A₂ is —O— In some embodiments, A₂ is a bond.

In some embodiments, A₃ is a bond, —O—, or —CR₅R₆—. In some embodiments, A₃ is —O— or —CR₅R₆—. In some embodiments, A₃ is —O—. In some embodiments, A₃ is —CR₅R₆—. In some embodiments, A₃ is a bond.

In some embodiments, A₄ is —CR₅R₆—.

In some embodiments, R₅ and R₆ are each independently H, halogen, or alkyl. In some embodiments, R₅ and R₆ are each independently H or alkyl. In some embodiments, the alkyl is methyl or ethyl.

In some embodiments, R₅ and R₆ are H. In some embodiments, R₅ and R₆ are halogen. In some embodiments, R₅ and R₆ together with the carbon atom to which they are attached form a carbocycle or heterocycle. In some embodiments, carbocycle is a C₃₋₆ cycloalkyl. In some embodiments, the heterocycle is a 3- or 6-membered heterocycle. In some embodiments, the heterocycle comprises 1 or 2 heteroatoms selected from the group consisting of N, O, and S.

In some embodiments, R₇ is H or alkyl.

In some embodiments, R₁ and R₂ are each independently H, halogen, or alkyl. In some embodiments, R₁ and R₂ are each independently H or alkyl. In some embodiments, the alkyl is methyl or ethyl. In some embodiments, R₁ and R₂ are H. In some embodiments, R₁ and R₂ are H or halogen. In some embodiments, halogen is fluoride. In some embodiments, R₁ and R₂ together with the carbon atom to which they are attached form a carbocycle or heterocycle. In some embodiments, the carbocycle is a C₃₋₆ cycloalkyl. In some embodiments, the heterocycle is a 3-or 6-membered heterocycle. In some embodiments, the heterocycle comprises 1 or 2 heteroatoms selected from the group consisting of N, O, and S.

In some embodiments, R₃ and R₄ are each independently H, halogen, or alkyl. In some embodiments, R₃ and R₄ are each independently H or alkyl. In some embodiments, the alkyl is methyl or ethyl. In some embodiments, R₃ and R₄ are H. In some embodiments, R₃ and R₄ are halogen. In some embodiments, the halogen is fluoride. In some embodiments, R₃ and R₄ together with the carbon atom to which they are attached form a carbocycle or heterocycle. In some embodiments, the carbocycle is a C₃₋₆ cycloalkyl. In some embodiments, the heterocycle is a 3- or 6-membered heterocycle. In some embodiments, the heterocycle comprises 1 or 2 heteroatoms selected from the group consisting of N, O, and S.

In some embodiments, \textcircled{B} is phenyl or 5- or 6-membered heteroaryl.

In some embodiments, V is —O— or —CR₈R₉—. In some embodiments, V is —O— or —NR₁₀—. In some embodiments, V is —O—. In some embodiments, V is —CR₈R₉—. In some embodiments, R₈ and R₉ are each independently H or alkyl. In some embodiments, R₈ and R₉ together with the carbon atom to which they are attached form a C₃₋₆ cycloalkyl. In some embodiments, R₁₀ is H, alkyl, —(C═O) alkyl, or —S(O), alkyl. In some embodiments, R₁₀ is H or alkyl. In some embodiments, the alkyl is methyl, ethyl, or isopropyl. In some embodiments, the alkyl is methyl.

In some embodiments, Y is a bond or —CR₈R₉—. In some embodiments, R₈ and R₉ are each independently H or alkyl. In some embodiments, R₈ and R₉ together with the carbon atom to which they are attached form a C₃₋₆ cycloalkyl.

In some embodiments, Z is —NR₁₀— or —CR₈R₉—. In some embodiments, Z is —NR₁₀—. In some embodiments, Z is —CR₈R₉—. In some embodiments, R₈ and R₉ are each independently H or alkyl. In some embodiments, R₈ and R₉ together with the carbon atom to which they are attached form a C₃₋₆ cycloalkyl. In some embodiments, R₁₀ is H, alkyl, —(C═O)alkyl, or —S(O)₂-alkyl. In some embodiments, R₁₀ is H or alkyl. In some embodiments, the alkyl is methyl, ethyl, or isopropyl. In some embodiments, the alkyl is methyl.

In some embodiments, X is —CR₁₁R₁₂—. In some embodiments, R₁₁ and R₁₂ are each independently H or alkyl. In some embodiments, the alkyl is methyl or ethyl. In some embodiments, R₁₁ and R₁₂ together with the carbon atom to which they are attached form a C₃₋₆ cycloalkyl.

In some embodiments, \textcircled{B} is optionally substituted phenyl. In some embodiments, the optionally substituted phenyl is:

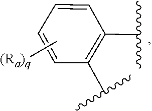

wherein Rₐ is halogen, alkyl, or alkoxy; and q is 0, 1, or 2. In some embodiments, \textcircled{B} is optionally substituted 5-membered heteroaryl. In some embodiments, \textcircled{B} is optionally substituted 6-membered heteroaryl. In some embodiments, the optionally substituted 6-membered heteroaryl is selected from the group consisting of pyridinyl, pyrazinyl, pyrimidinyl, or pyridazinyl. In some embodiments, the heteroaryl is optionally substituted with one or more halogen, alkyl, alkoxy, or combination thereof. In some embodiments, the optionally substituted 6-membered heteroaryl is:

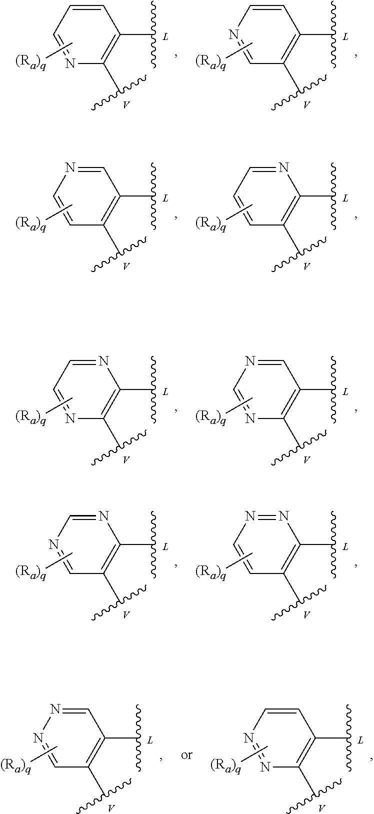

wherein Rₐ is halogen, alkyl, or alkoxy; and q is 0, 1, or 2. In some embodiments, Rₐ is halogen or alkyl. In some embodiments, the halogen is F or Cl. In some embodiments, the alkyl is methyl. In some embodiments, q is 0 or 1. In some embodiments, q is 0.

In some embodiments, m is 0 or 1. In some embodiments, m is 0.

In some embodiments, n is 0 or 1. In some embodiments, n is 1.

In some embodiments, p is 0 or 1. In some embodiments, p is 0. In some embodiments, p is 1.

In some embodiments, L is a -carbocyclyl-O— or -heterocyclyl-O— linker having the structure

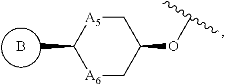

wherein A₅ and A₆ are each independently —O— or —CH₂—. In some embodiments, A₅ is —O—. In some embodiments, As is —CH₂—. In some embodiments, A₆ is —O—. In some embodiments, A₆ is —CH₂—.

In some embodiments, L is

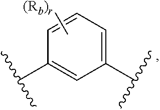

wherein R_{b} is halogen, alkyl, or alkoxy; and r is 0, 1, or 2. In some embodiments, R_{b} is halogen. In some embodiments, the halogen is fluoride. In some embodiments, r is 1. In some embodiments, r is 0. In some embodiments, L is

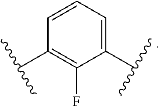 [SMILES: *c1cccc(*)c1F]

In some embodiments, L is a 5- or 6-membered heteroaryl linker. In some embodiments, L is a 5- or 6-membered heteroaryl linker having 1 or 2 nitrogen atoms. In some embodiments, L is

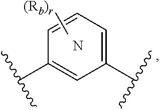

wherein R_{b} is halogen, alkyl, or alkoxy; and r is 0 or 1.

In some embodiments, the present disclosure provides a compound selected from the group consisting of:

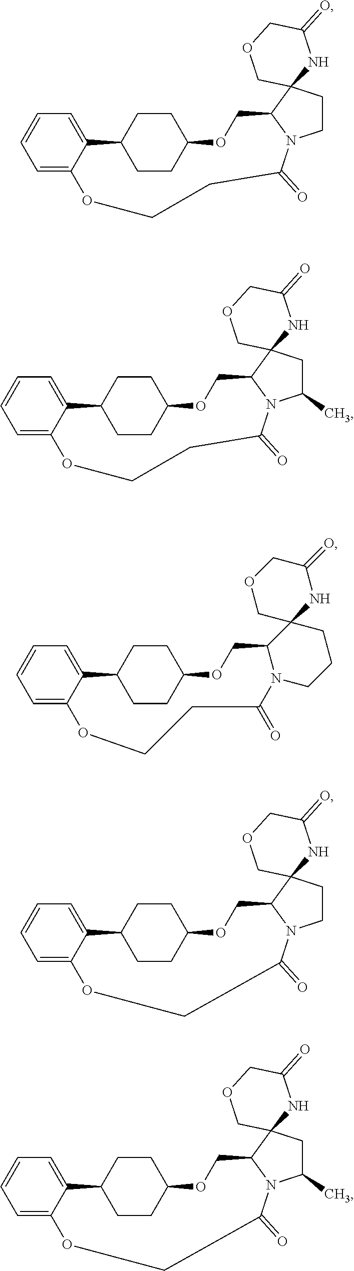 [SMILES: O=C1COC[C@@]2(CCN3C(=O)CCCCOc4ccccc4[C@H]4CC[C@H](CC4)OC[C@H]32)N1 ; C[C@@H]1C[C@@]2(COCC(=O)N2)[C@@H]2CO[C@H]3CC[C@H](CC3)c3ccccc3OCCCCC(=O)N12 ; O=C1COC[C@@]2(CCCN3C(=O)CCCCOc4ccccc4[C@H]4CC[C@H](CC4)OC[C@H]32)N1 ; O=C1COC[C@@]2(CCN3C(=O)CCCOc4ccccc4[C@H]4CC[C@H](CC4)OC[C@H]32)N1 ; C[C@@H]1C[C@@]2(COCC(=O)N2)[C@@H]2CO[C@H]3CC[C@H](CC3)c3ccccc3OCCCC(=O)N12]

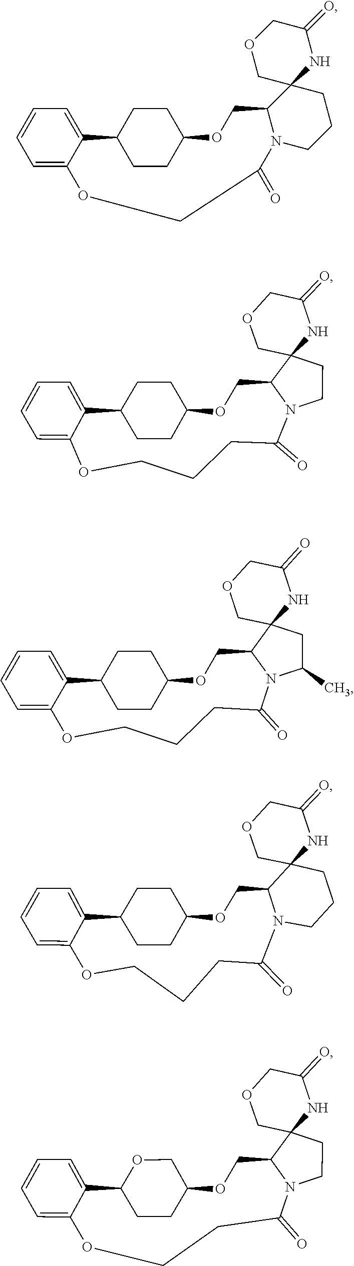 [SMILES: O=C1COC[C@@]2(CCCN3C(=O)CCCOc4ccccc4[C@H]4CC[C@H](CC4)OC[C@H]32)N1 ; O=C1COC[C@@]2(CCN3C(=O)CCCOc4ccccc4[C@H]4CC[C@H](CC4)OC[C@H]32)N1 ; C[C@@H]1C[C@@]2(COCC(=O)N2)[C@@H]2CO[C@H]3CC[C@H](CC3)c3ccccc3OCCCC(=O)N12 ; O=C1COC[C@@]2(CCCN3C(=O)CCCOc4ccccc4[C@H]4CC[C@H](CC4)OC[C@H]32)N1 ; O=C1COC[C@@]2(CCN3C(=O)CCOc4ccccc4[C@@H]4CC[C@@H](CO4)OC[C@H]32)N1]

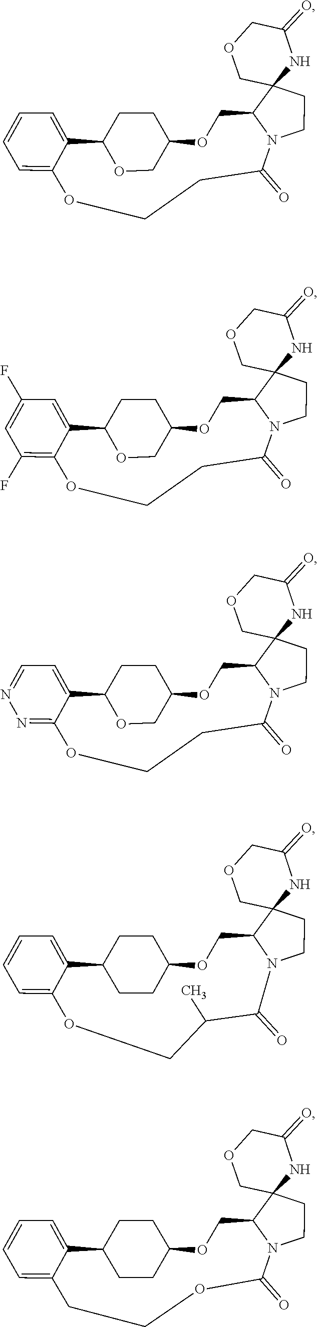 [SMILES: O=C1COC[C@@]2(CCN3C(=O)CCOc4ccccc4[C@H]4CC[C@H](CO4)OC[C@H]32)N1 ; O=C1COC[C@@]2(CCN3C(=O)CCOc4c(F)cc(F)cc4[C@H]4CC[C@H](CO4)OC[C@H]32)N1 ; O=C1COC[C@@]2(CCN3C(=O)CCOc4nnccc4[C@H]4CC[C@H](CO4)OC[C@H]32)N1 ; CC1COc2ccccc2[C@H]2CC[C@H](CC2)OC[C@@H]2N(CC[C@@]23COCC(=O)N3)C1=O ; O=C1COC[C@@]2(CCN3C(=O)OCCc4ccccc4[C@H]4CC[C@H](CC4)OC[C@H]32)N1]

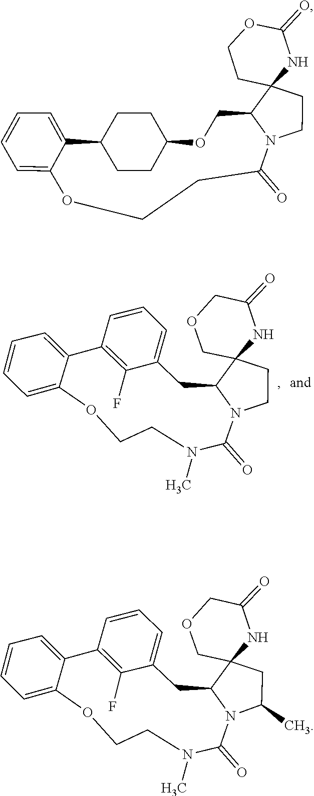

In some embodiments, the present disclosure provides a pharmaceutical composition comprising a compound disclosed herein (e.g., a compound of Formula (I), Formula (I-1), Formula (I-2), Formula (IA), Formula (IA-1), Formula (IA-1-1), Formula (IA-2), Formula (IA-2-1), Formula (IA-3), Formula (IA-4), Formula (IA-5), Formula (IB), Formula (IB-1), Formula (IB-2), Formula (IC), Formula (IC-1), Formula (IC-2), Formula (IC-3), Formula (IC-4), Formula (IC-5), Formula (IC-6), Formula (ID), Formula (ID-1), Formula (ID-2), Formula (IE), Formula, (IF), Table 1, or Table 2) or a pharmaceutically acceptable salt thereof, and a pharmaceutically acceptable excipient.

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The terms “admin ister,” “administering” or “administration” as used herein refer to administering a compound or pharmaceutically acceptable salt of the compound or a composition or formulation comprising the compound or pharmaceutically acceptable salt of the compound to a patient.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tr is -(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Those skilled in the art will further recognize that acid addition salts may be prepared by reaction of the compounds with the appropriate inorganic or organic acid via any of a number of known methods.

The term “treating” as used herein with regard to a patient, refers to improving at least one symptom of the patient's disorder. Treating can be improving, or at least partially ameliorating a disorder or an associated symptom of a disorder.

The terms “effective amount” and “therapeutically effective amount” are used interchangeably in this disclosure and refer to an amount of a compound, or a salt thereof, (or pharmaceutical composition containing the compound or salt) that, when administered to a patient, is capable of performing the intended result. The “effective amount” can vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired clinical benefit after administration to a patient in need thereof.

The term “carrier” or “vehicle” as used interchangeably herein encompasses carriers, excipients, adjuvants, and diluents or a combination of any of the foregoing, meaning a material, composition or vehicle, such as a liquid or solid filler, diluent, excipient, solvent or encapsulating material involved in carrying or transporting a pharmaceutical agent from one organ, or portion of the body, to another organ or portion of the body. In addition to the adjuvants, excipients and diluents known to one skilled in the art, the carrier includes nanoparticles of organic and inorganic nature.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, and the like. The alkylene chain is attached to the rest of the molecule through a single bond and to a radical group (e.g., those described herein) through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain having from two to twelve carbon atoms and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to an unsaturated, straight or branched divalent hydrocarbon chain radical having one or more olefins and from two to twelve carbon atoms. Non-limiting examples of C₂-C₁₂ alkenylene include ethenylene, propenylene, n-butenylene, and the like. The alkenylene chain is attached to the rest of the molecule through a single bond and to a radical group (e.g., those described herein) through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to an unsaturated, straight or branched divalent hydrocarbon chain radical having one or more alkynes and from two to twelve carbon atoms. Non-limiting examples of C₂-C₁₂ alkynylene include ethynylene, propynylene, n-butynylene, and the like. The alkynylene chain is attached to the rest of the molecule through a single bond and to a radical group (e.g., those described herein) through a single bond. The points of attachment of the alkynylene chain to the rest of the molecule and to the radical group can be through any two carbons within the chain having a suitable valency. Unless stated otherwise specifically in the specification, an alkynylene chain can be optionally substituted.

“Alkoxy” refers to a group of the formula —ORₐ where Rₐ is an alkyl, alkenyl or alkynyl as defined above containing one to twelve carbon atoms. Unless stated otherwise specifically in the specification, an alkoxy group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

“Aralkyl” or “arylalkyl” refers to a radical of the formula —R_{b}—R_{c} where R_{b} is an alkylene group as defined above and R_{c} is one or more aryl radicals as defined above, for example, benzyl, diphenylmethyl and the like. Unless stated otherwise specifically in the specification, an aralkyl group can be optionally substituted.

“Carbocyclyl,” “carbocyclic ring” or “carbocycle” refers to a rings structure, wherein the atoms which form the ring are each carbon, and which is attached to the rest of the molecule by a single bond. Carbocyclic rings can comprise from 3 to 20 carbon atoms in the ring. Carbocyclic rings include aryls and cycloalkyl, cycloalkenyl, and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon consisting solely of carbon and hydrogen atoms, which can include fused, bridged, or spirocyclic ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyls include, for example, cyclopentenyl, cyclohexenyl, cycloheptenyl, cycloctenyl, and the like. Polycyclic cycloalkenyls include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl include, for example, cycloheptynyl, cyclooctynyl, and the like. Unless otherwise stated specifically in the specification, a cycloalkynyl group can be optionally substituted.

“Haloalkyl” refers to an alkyl, as defined above, that is substituted by one or more halo radicals, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Heterocyclyl,” “heterocyclic ring” or “heterocycle” refers to a stable saturated or unsaturated 3- to 20-membered ring which consists of two to nineteen carbon atoms and from one to six heteroatoms selected from the group consisting of nitrogen, oxygen and sulfur, and which is attached to the rest of the molecule by a single bond. Heterocyclyl or heterocyclic rings include heterocyclylalkyls, heterocyclylalkenyls, and hetercyclylalkynyls. Unless stated otherwise specifically in the specification, the heterocyclyl can be a monocyclic, bicyclic, tricyclic or tetracyclic ring system, which can include fused, bridged, or spirocyclic ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heteroaryl” refers to a 5- to 20-membered ring system comprising hydrogen atoms, one to nineteen carbon atoms, one to six heteroatoms selected from the group consisting of nitrogen, oxygen and sulfur, at least one aromatic ring, and which is attached to the rest of the molecule by a single bond. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“Heterocyclylalkyl” refers to a radical of the formula —R_{b}—R_{c} where R_{b} is an alkylene, alkenylene, or alkynylene group as defined above and Re is a heterocyclyl radical as defined above. Unless stated otherwise specifically in the specification, a heterocyclylalkyl group can be optionally substituted.

The term “substituted” used herein means any of the groups described herein (e.g., alkyl, alkenyl, alkynyl, alkoxy, aryl, aralkyl, carbocyclyl, cycloalkyl, cycloalkenyl, cycloalkynyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher- order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, =NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol “ [symbol] ” (hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example, “XY [symbol] ” indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or “XY [symbol] ” infers that when R₃ is “XY”, the point of attachment bond is the same bond as the bond by which R₃ is depicted as being bonded to CH₃.

### Compounds

The present disclosure provides macrocyclic compounds that are agonists of the orexin type 2 receptor as well as pharmaceutical compositions thereof and uses thereof in treating various diseases and disorders.

In one aspect, the present disclosure provides a compound of Formula (I):

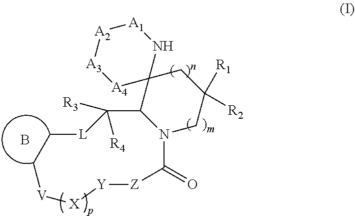

or a pharmaceutically acceptable salt or stereoisomer thereof,
wherein:
L is a linker selected from the group consisting of aryl, heteroaryl, -carbocyclyl-O—, and -heterocyclyl-O—;
A₁ is —C(O)—, —S(O)₂—, or —C(H)(CF₃)—;
A₂ and A₃ are each independently a bond, —O—, —CR₅R₆—, —NR₇—, or —S—; or A₂ and A₃ together with an optionally substituted carbon atom form a cyclopropyl ring having the structure:

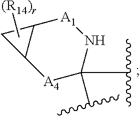

A₄ is a bond, —O—, CR₅R₆—, —NR₇—, —S—, —(CR₅R₆)₂—, —CR₅R₆—O—, —CR₅R₆—S—, —CR₅R₆—N(R₇)—, —O—CR₅R₆—, —S—CR₅R₆—, or —N(R₇)—CR₅R₆—, with the proviso that the ring that includes A₂, A₃ and A₄ does not contain-O—O—, —O—NR₇— or —NR₇—NR₇—;
\textcircled{B} is phenyl, 5- or 6-membered heteroaryl, cycloalkyl, or heterocyclyl, each of which is optionally substituted;
V and Z are each independently —O—, —CR₈R₉—, or —NR₁₀—;
X is —O—, —CR₁₁R₁₂—, or —NR₁₃—;
Y is a bond, —O—, —CR₈R₉—, or —NR₁₀—;
R₁, R₂, R₃, R₄, R₅, R₆, R₈, R₉, R₁₁, R₁₂ and R₁₄ are each independently hydrogen, halogen, alkyl, cycloalkyl, or heterocyclyl; and/or R₁ and R₂ together with the atom to which they are attached form a carbocycle or heterocycle; and/or R₃ and R₄ together with the atom to which they are attached form a carbocycle or heterocycle; and/or R₅ and R₆ together with the atom to which they are attached form a carbocycle or heterocycle; and/or R₈ and R₉ together with the atom to which they are attached form a carbocycle or heterocycle; and/or R₁₁ and R₁₂ together with the atom to which they are attached form a carbocycle or heterocycle;
R₇, R₁₀, and R₁₃ are each independently hydrogen, alkyl, cycloalkyl, heterocyclyl, aryl, heteroaryl, —(C═O) alkyl, —(C═O) cycloalkyl, —(C═O) heterocyclyl, —(C═O)—O-alkyl, —(C═O)—O— cycloalkyl, —(C═O)—O-heterocyclyl, —(C═O)—O-heteroaryl, —S(O)₂-alkyl, —S(O)₂-cycloalkyl, or —S(O)₂-heterocyclyl; and
m, n, p, and r are each independently 0, 1, or 2.

In another aspect, the present disclosure provides a compound of Formula (I):

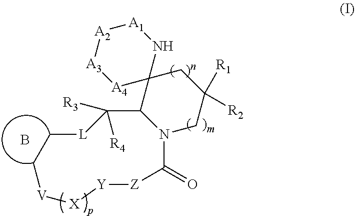

or a pharmaceutically acceptable salt or stereoisomer thereof,
wherein:
L is a linker selected from the group consisting of aryl, heteroaryl, -carbocyclyl-O—, and -heterocyclyl-O—;
A₁ is —C(O)— or —S(O)₂—;
A₂ and A₃ are each independently a bond, —O—, —CR₅R₆—, —NR₇—, or —S—; or A₂ and A₃ together with an optionally substituted carbon atom form a cyclopropyl ring having the structure:

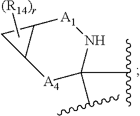

A₄ is a bond, —O—, —CR₅R₆—, —NR₇—, —S—, —(CR₅R₆)₂—, —CR₅R₆—O—, —CR₅R₆—S—, —CR₅R₆—N(R₇)—, —O—CR₅R₆—, —S—CR₅R₆—, or —N(R₇)—CR₅R₆—, with the proviso that the ring that includes A₂, A₃ and A₄ does not contain —O—O—, —O—NR₇— or —NR₇—NR₇—;
\textcircled{B} is phenyl, 5- or 6-membered heteroaryl, cycloalkyl, or heterocyclyl, each of which is optionally substituted;
V and Z are each independently —O—, —CR₈R₉—, or —NR₁₀—;
X is —O—, —CR₁₁R₁₂—, or —NR₁₃—;
Y is a bond, —O—, —CR₈R₉—, or —NR₁₀—;
R₁, R₂, R₃, R₄, R₅, R₆, R₈, R₉, R₁₁, R₁₂ and R₁₄ are each independently hydrogen, halogen, alkyl, cycloalkyl, or heterocyclyl; and/or R₁ and R₂ together with the atom to which they are attached form a carbocycle or heterocycle; and/or R₃ and R₄ together with the atom to which they are attached form a carbocycle or heterocycle; and/or R₅ and R₆ together with the atom to which they are attached form a carbocycle or heterocycle; and/or R₈ and R₉ together with the atom to which they are attached form a carbocycle or heterocycle; and/or R₁₁ and R₁₂ together with the atom to which they are attached form a carbocycle or heterocycle;
R₇, R₁₀, and R₁₃ are each independently hydrogen, alkyl, cycloalkyl, heterocyclyl, aryl, heteroaryl, —(C═O)alkyl, —(C═O)cycloalkyl, —(C═O)heterocyclyl, —(C═O)—O-alkyl, —(C═O)—O-cycloalkyl, —(C═O)—O-heterocyclyl, —(C—O)—O-heteroaryl, —S(O)₂-alkyl, —S(O)₂-cycloalkyl, or —S(O)₂-heterocyclyl; and
m, n, p, and r are each independently 0, 1, or 2.

In some embodiments, L is a linker selected from the group consisting of aryl, heteroaryl, -carbocyclyl-O—, and -heterocyclyl-O—, wherein-carbocyclyl-O— and -heterocyclyl-O— have the following orientation:

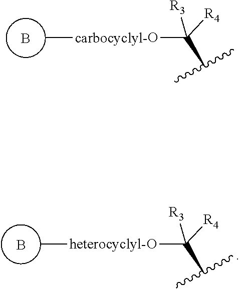

In some embodiments, L is a linker selected from the group consisting of aryl, heteroaryl, -cycloalkyl-O—, and -heterocyclyl-O—, wherein-cycloalkyl-O— and -heterocyclyl-O— have the following orientation:

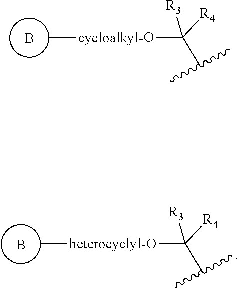

In some embodiments, the present disclosure provides a compound of Formula (I-1):

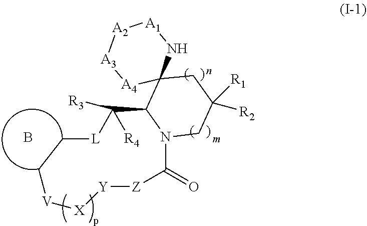

or a pharmaceutically acceptable salt or stereoisomer thereof, wherein m, n, p, A₁, A₂, A₃, A₄, R₁, R₂, R₃, R₄, L, \textcircled{B}, V, X, Y and Z are as defined herein.

In some embodiments, the present disclosure provides a compound of Formula (I-2):

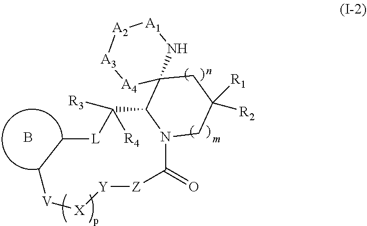

or a pharmaceutically acceptable salt or stereoisomer thereof, wherein m, n, p, A₁, A₂, A₃, A₄, R₁, R₂, R₃, R₄, L, \textcircled{B}, V, X, Y and Z are as defined herein.

In some embodiments, the present disclosure provides a compound of Formula (IA):

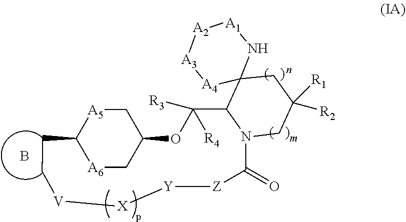

or a pharmaceutically acceptable salt or stereoisomer thereof, wherein m, n, p, A₁, A₂, A₃, A₄, A₅, A₆, R₁, R₂, R₃, R₄, \textcircled{B}, V, X, Y and Z are as defined herein.

In some embodiments, the present disclosure provides a compound of Formula (IA-1):

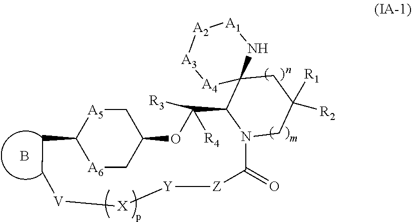

or a pharmaceutically acceptable salt or stereoisomer thereof, wherein m, n, p, A₁, A₂, A₃, A₄, A₅, A₆, R₁, R₂, R₃, R₄, \textcircled{B}, V, X, Y and Z are as defined herein.

In some embodiments, the present disclosure provides a compound of Formula (IA-1-1):

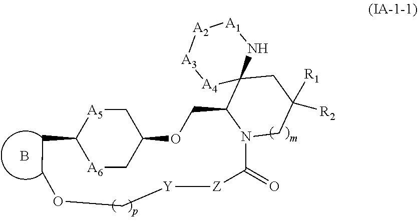

or a pharmaceutically acceptable salt or stereoisomer thereof, wherein m, p, A₁, A₂, A₃, A₄, R₁, R₂, \textcircled{B}, Y, and Z are as defined herein.

In some embodiments, the present disclosure provides a compound of Formula (IA-2):

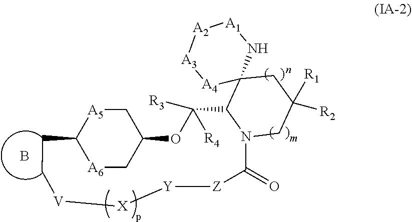

or a pharmaceutically acceptable salt or stereoisomer thereof, wherein m, n, p, A₁, A₂, A₃, A₄, A₅, A₆, R₁, R₂, R₃, R₄, \textcircled{B}, V, X, Y and Z are as defined herein.

In some embodiments, the present disclosure provides a compound of Formula (IA-2-1):

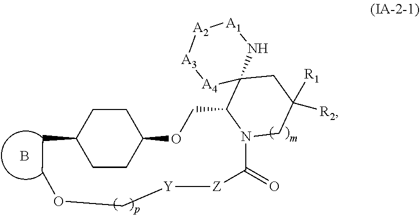

or a pharmaceutically acceptable salt or stereoisomer thereof, wherein m, p, A₁, A₂, A₃, A₄, R₁, R₂, \textcircled{B}, Y, and Z are as defined herein.

In some embodiments, the present disclosure provides a compound of Formula (IA-3):

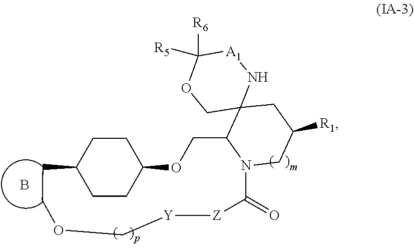

or a pharmaceutically acceptable salt or stereoisomer thereof, wherein m, p, A₁, R₁, R₅, R₆, \textcircled{B} Y, and Z are as defined herein.

In some embodiments, the present disclosure provides a compound of Formula (IA-4):

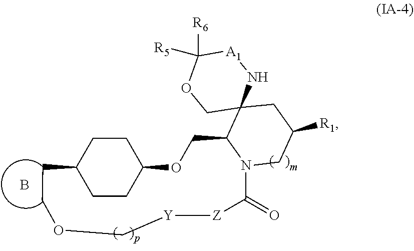

or a pharmaceutically acceptable salt thereof, wherein m, p, A₁, R₁, R₅, R₆, \textcircled{B}, Y, and Z are as defined herein.

In some embodiments, the present disclosure provides a compound of Formula (IA-5):

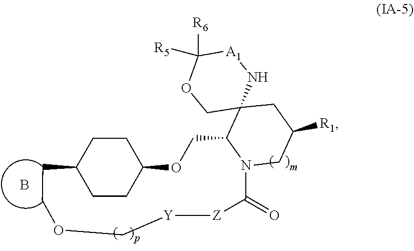

or a pharmaceutically acceptable salt or stereoisomer thereof, wherein m, p, A₁, R₁, R₅, R₆, \textcircled{B}, Y, and Z are as defined herein.

In some embodiments, the present disclosure provides a compound of Formula (IB):

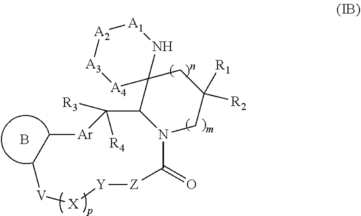

or a pharmaceutically acceptable salt or stereoisomer thereof, wherein m, n, p, A₁, A₂, A₃, A₄, R₁, R₂, R₃, R₄, Ar, \textcircled{B}, V, X, Y and Z are as defined herein.

In some embodiments, the present disclosure provides a compound of Formula (IB-1):

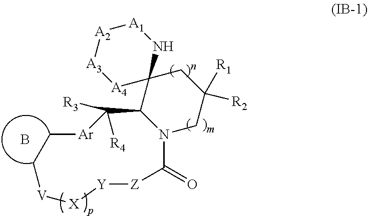

or a pharmaceutically acceptable salt or stereoisomer thereof, wherein m, n, p, A₁, A₂, A₃, A₄, R₁, R₂, R₃, R₄, Ar, \textcircled{B}, V, X, Y and Z are as defined herein.

In some embodiments, the present disclosure provides a compound of Formula (IB-2):

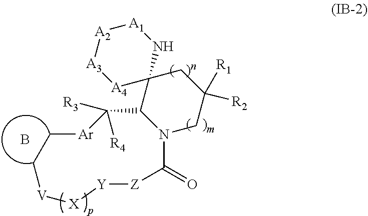

or a pharmaceutically acceptable salt or stereoisomer thereof, wherein m, n, p, A₁, A₂, A₃, A₄, R₁, R₂, R₃, R₄, Ar, \textcircled{B}, V, X, Y and Z are as defined herein.

In some embodiments, the present disclosure provides a compound of Formula (IC):

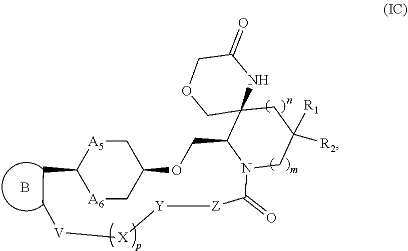

or a pharmaceutically acceptable salt or stereoisomer thereof, wherein m, n, p, A₅, A₆, R₁, R₂, \textcircled{B}, V, X, Y and Z are as defined herein.

In some embodiments, the present disclosure provides a compound of Formula (IC-1):

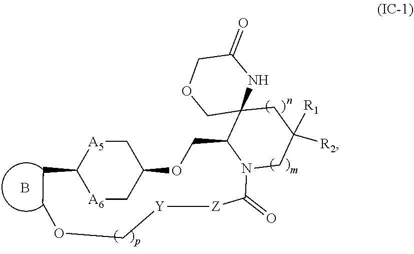

or a pharmaceutically acceptable salt or stereoisomer thereof, wherein m, n, p, A₅, A₆, R₁, R₂, \textcircled{B}, Y and Z are as defined herein.

In some embodiments, the present disclosure provides a compound of Formula (IC-2):

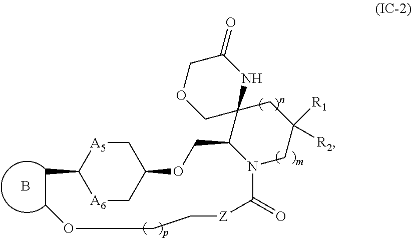

or a pharmaceutically acceptable salt or stereoisomer thereof, wherein m, n, p, A₅, A₆, R₁, R₂, \textcircled{B}, and Z are as defined herein.

In some embodiments, the present disclosure provides a compound of Formula (IC-3):

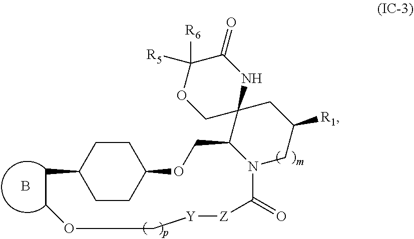

or a pharmaceutically acceptable salt or stereoisomer thereof, wherein m, p, R₁, R₅, R₆, \textcircled{B}, Y, and Z are as defined herein.

In some embodiments, the present disclosure provides a compound of Formula (IC-4):

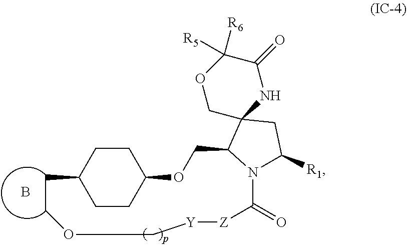

or a pharmaceutically acceptable salt or stereoisomer thereof, wherein p, R₁, R₅, R₆, \textcircled{B}, Y, and Z are as defined herein.

In some embodiments, the present disclosure provides a compound of Formula (IC-5):

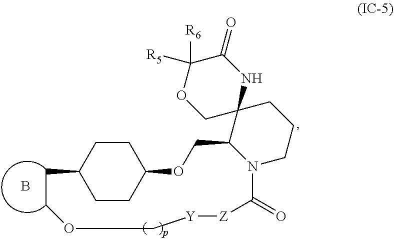

or a pharmaceutically acceptable salt or stereoisomer thereof, wherein p, R₅, R₆, \textcircled{B}, Y, and Z are as defined herein.

In some embodiments, the present disclosure provides a compound of Formula (IC-6):

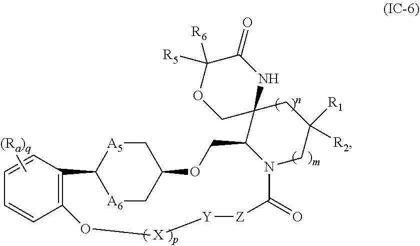

or a pharmaceutically acceptable salt or stereoisomer thereof, wherein m, n, p, q, Rₐ, R₁, R₂, R₅, R₆, X, Y and Z are as defined herein.

In some embodiments, the present disclosure provides a compound of Formula (ID):

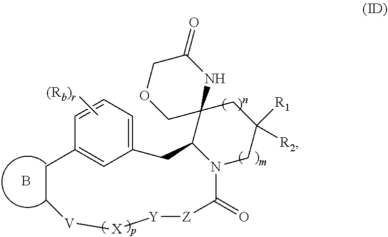

or a pharmaceutically acceptable salt or stereoisomer thereof, wherein m, n, p, r, R₁, R₂, R_{b}, \textcircled{B}, V, X, Y and Z are defined herein.

In some embodiments, the present disclosure provides a compound of Formula (ID-1):

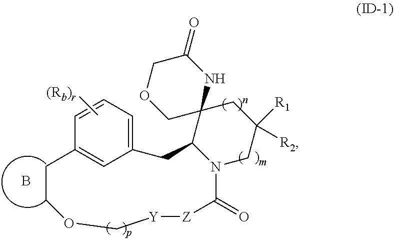

or a pharmaceutically acceptable salt or stereoisomer thereof, wherein m, n, p, r, R₁, R₂, R_{b}, \textcircled{B} Y, and Z are defined herein.

In some embodiments, the present disclosure provides a compound of Formula (ID-2):

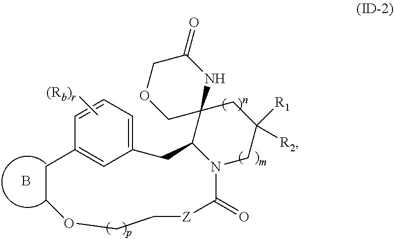

or a pharmaceutically acceptable salt or stereoisomer thereof, wherein m, n, p, r, R₁, R₂, R_{b}, \textcircled{B} and Z are defined herein.

In some embodiments, the present disclosure provides a compound of Formula (IE):

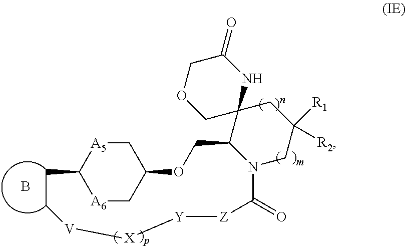

or a pharmaceutically acceptable salt or stereoisomer thereof, wherein m, n, p, A₅, A₆, R₁, R₂, \textcircled{B}, V, X, Y and Z are as defined herein.

In some embodiments, the present disclosure provides a compound of Formula (IF):

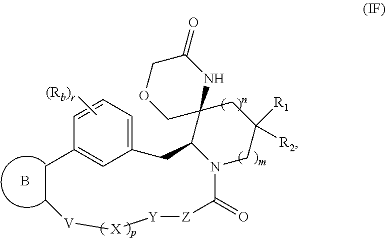

or a pharmaceutically acceptable salt or stereoisomer thereof, wherein m, n, p, r, R₁, R₂, R_{b}, \textcircled{B}, V, X, Y and Z are defined herein.

In some embodiments, the stereoisomer is a diastereoisomer of the compound. In some embodiments, the stereoisomer is an enantiomer of the compound.

In some embodiments, L is a linker selected from the group consisting of aryl, —carbocyclyl-O—, and -heterocyclyl-O—. In some embodiments, L is -carbocyclyl-O— or -heterocyclyl-O—. In some embodiments, L is -carbocyclyl-O—. In some embodiments, L is -heterocyclyl-O—. In some embodiments, the carbocyclyl is a C₃₋₆ cycloalkyl. In some embodiments, the carbocyclyl is cyclohexyl. In some embodiments, the carbocyclyl is

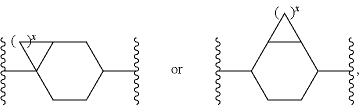

wherein x is 1, 2, 3, or 4. In some embodiments, the heterocyclyl is a 4- to 6-membered heterocyclyl. In some embodiments, the heterocycle comprises 1 or 2 heteroatoms selected from the group consisting of N, O, and S. In some embodiments, L is a -carbocyclyl-O— or -heterocyclyl-O— linker having the structure

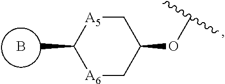

wherein A₅ and A₆ are each independently —O— or —CH₂—. In some embodiments, As is —O—. In some embodiments, As is —CH₂—. In some embodiments. A₆ is —O—. In some embodiments. A₆ is —CH₂—. In some embodiments, L has the structure

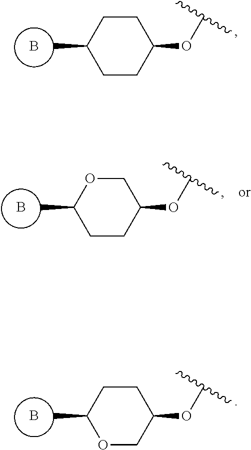

In some embodiments, L has the structure

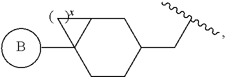

wherein x is 1, 2, 3, or 4. In some embodiments, L has the structure

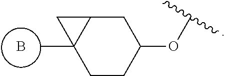

In some embodiments, L has the structure

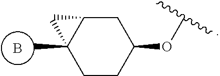

In some embodiments, A₁ is —C(O)— or —S(O)₂—. In some embodiments, A₁ is —C(O)—. In some embodiments, A₁ is —S(O)₂—. In some embodiments, A₁ is —C(H)(CF₃)—.

In some embodiments, A₂, A₃ and A₄ are each independently —O—, —CR₅R₆—, —NR₇—, or —S—, with the proviso that the ring that includes A₂, A₃ and A₄ does not contain —O—O—, —O—S—, —S—S—, —O—NR₇—, —S—NR₇—, or —NR₇—NR₇—. In some embodiments, A₂, A₃ and A₄ are each independently —O—, —CR₅R₆—, —NR₇—, or —S—, with the proviso that the ring that includes A₂, A₃ and A₄ does not contain —O—O—, —O—NR₇— or —NR₇—NR₇—. In some embodiments, A₂, A₃ and A₄ are each independently —O—, —CR₅R₆—, or —NR₇—, with the proviso that the ring that includes A₂, A₃ and A₄ does not contain —O—O—, —O—NR₇— or —NR₇—NR₇—. In some embodiments, A₂ and A₃ together with an optionally substituted carbon atom form a cyclopropyl ring having the structure:

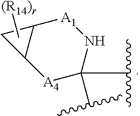

In some embodiments, A₂ and A₃ together with an optionally substituted carbon atom form a cyclopropyl ring having the structure:

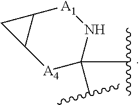

In some embodiments, A₂ is —O—, —NR₇—, or —CR₅R₆—. In some embodiments, A₂ is a bond, —O—, or —CR₅R₆—. In some embodiments, A₂ is —O— or —CR₅R₆—. In some embodiments, A₂ is —O— or —NR₇—. In some embodiments, A₂ is —CR₅R₆—. In some embodiments, A₂ is —O—. In some embodiments A₂ is a bond.

In some embodiments, A₃ is —O—, —NR₇—, or —CR₅R₆—. In some embodiments, A₃ is a bond, —O—, or —CR₅R₆—. In some embodiments, A₃ is —O— or —CR₅R₆—. In some embodiments, A₃ is —O— or —NR₇—. In some embodiments, A₃ is —O—. In some embodiments, A₃ is —CH₂—. In some embodiments A₃ is a bond.

In some embodiments, A₄ is a bond, —O—, —CR₅R₆—, —NR₇—, —(CR₅R₆)₂—, —CR₅R₆—O—, —CR₅R₆—N(R₇)—, —O—CR₅R₆—, or —N(R₇)—CR₅R₆—. In some embodiments, A₄ is a bond, —O—, —CR₅R₆—, —(CR₅R₆)₂—, —CR₅R₆—O—, or —O—CR₅R₆—. In some embodiments, A₄ is —O—, —NR₇—, or —CR₅R₆—. In some embodiments, A₄ is —O— or —NR₇—. In some embodiments, A₄ is —O— or —CR₅R₆—. In some embodiments, A₄ is —CR₅R₆—. In some embodiments, A₄ is —O—.

In some embodiments, A₂ is —O—, —NR₇—, or —S—, and A₃ and A₄ are each —CR₅R₆—. In some embodiments, A₂ is —O—, and A₃ and A₄ are each —CR₅R₆—. In some embodiments, A₂ is —O—, A₃ is —CR₅R₆—, and A₄ is a bond. In some embodiments, A₂ is —CR₅R₆—, A₃ is —O—, and A₄ is a bond. In some embodiments, A₃ is —O—, —NR₇—, or —S—, and A₂ and A₄ are each —CR₅R₆—. In some embodiments, A₃ is —O—, and A₂ and A₄ are each —CR₅R₆—. In some embodiments, A₄ is —O—, —NR₇—, or —S—, and A₂ and A₃ are each —CR₅R₆—. In some embodiments, A₄ is —O—, and A₂ and A₃ are each —CR₅R₆—.

In some embodiments, A₁ is —C(O)—, —S(O)₂—, or —C(H)(CF₃)—; A₂ is —O— or —CR₅R₆—; A₃ is —O— or —CR₅R₆—; and A₄ is a bond or —CH₂—. In some embodiments, A₁ is —C(O)— or —S(O)₂—; A₂ is —O— or —CR₅R₆—; A₃ is —O— or —CR₅R₆—; and A₄ is a bond or —CH₂—. In some embodiments, A₁ is —C(O)— or —S(O)₂—; A₂ is —O—; A₃ is —CR₅R₆—; and A₄ is a bond or —CH₂—. In some embodiments, A₁ is —C(O)— or —S(O)₂—; A₂ is —CR₅R₆—; A₃ is —O—; and A₄ is a bond or —CH₂—.

In some embodiments, the ring that includes A₂, A₃ and A₄ does not contain —O—O—, —O— NR₇— or —NR₇—NR₇—. In some embodiments, the ring that includes A₂, A₃ and A₄ does not contain two adjacent heteroatoms.

In some embodiments, R₁, R₂, R₃, R₄, R₅, R₆, R₈, R₉, R₁₁, R₁₂, and R₁₄ are each independently hydrogen, halogen, alkyl, or cycloalkyl. In some embodiments, R₁, R₂, R₃, R₄, R₅, R₆, R₈, R₉, R₁₁, R₁₂ and R₁₄ are each independently hydrogen, halogen, or alkyl. In some embodiments, the alkyl is a C₁₋₅alkyl. In some embodiments, the alkyl is methyl, ethyl, or isopropyl. In some embodiments, the cycloalkyl is a C₃₋₆cycloalkyl. In some embodiments, the cycloalkyl is a cyclopropyl.

In some embodiments, R₁ and R₂ are each independently H, halogen, or alkyl. In some embodiments, R₁ and R₂ are each independently H or alkyl. In some embodiments, R₁ and R₂ are alkyl. In some embodiments, the alkyl is methyl or ethyl. In some embodiments, the alkyl is methyl. In some embodiments, R₁ is methyl and R₂ is H. In some embodiments, R₁ and R₂ are H. In some embodiments, R₁ and R₂ are each independently H or halogen. In some embodiments, halogen is fluoride. In some embodiments, R₁ and R₂ together with the carbon atom to which they are attached form a carbocycle or heterocycle. In some embodiments, the carbocycle is a C₃₋₆ cycloalkyl. In some embodiments, the carbocycle is a cyclopropyl. In some embodiments, the heterocycle is a 3- or 6-membered heterocycle. In some embodiments, the heterocycle comprises 1 or 2 heteroatoms selected from the group consisting of N, O, and S.

In some embodiments, R₃ and R₄ are each independently H, halogen, or alkyl. In some embodiments, R₃ and R₄ are each independently H or alkyl. In some embodiments, R₃ and R₄ are alkyl. In some embodiments, the alkyl is methyl or ethyl. In some embodiments, R₃ and R₄ are each independently H or halogen. In some embodiments, R₃ and R₄ are H. In some embodiments, R₃ and R₄ are halogen. In some embodiments, the halogen is fluoride. In some embodiments, R₃ and R₄ together with the carbon atom to which they are attached form a carbocycle or heterocycle. In some embodiments, the carbocycle is a C₃₋₆ cycloalkyl. In some embodiments, the carbocycle is a cyclopropyl. In some embodiments, the heterocycle is a 3- or 6-membered heterocycle. In some embodiments, the heterocycle comprises 1 or 2 heteroatoms selected from the group consisting of N, O, and S.

In some embodiments, R₅ and R₆ are each independently H, halogen, or alkyl. In some embodiments, R₅ and R₆ are each independently H or alkyl. In some embodiments, R₅ and R₆ are alkyl. In some embodiments, R₅ and R₆ are each independently H or halogen. In some embodiments, the alkyl is a haloalkyl. In some embodiments, the haloalkyl is CF₃. In some embodiments, the alkyl is methyl or ethyl. In some embodiments, R₅ and R₆ are each independently H, F, or CF₃. In some embodiments, R₅ and R₆ are H. In some embodiments, R₅ and R₆ are halogen. In some embodiments, the halogen is fluoride. In some embodiments, R₅ and R₆ together with the carbon atom to which they are attached form a carbocycle or heterocycle. In some embodiments, the carbocycle is a C₃₋₆ cycloalkyl. In some embodiments, the carbocycle is a cyclopropyl. In some embodiments, the heterocycle is a 3- or 6-membered heterocycle. In some embodiments, the heterocycle comprises 1 or 2 heteroatoms selected from the group consisting of N, O, and S.

In some embodiments, R₇, R₁₀, and R₁₃ are each independently hydrogen, alkyl, cycloalkyl, heterocyclyl, aryl, heteroaryl, —(C═O) alkyl, —(C═O) cycloalkyl, —(C═O)—O-alkyl, —(C═O)—O— cycloalkyl, —S(O)₂-alkyl, or —S(O)₂-cycloalkyl. In some embodiments, R₇, R₁₀, R₁₃, and R₁₄ are each independently hydrogen, alkyl, cycloalkyl, —(C—O) alkyl, —(C—O) cycloalkyl, —(C═O)—O— alkyl, —(C═O)—O-cycloalkyl, —S(O)₂-alkyl, —S(O)₂-cycloalkyl, or —S(O)₂-heterocyclyl. In some embodiments, R₇, R₁₀, R₁₃, and R₁₄ are each independently hydrogen, alkyl, —(C═O) alkyl, or —S(O)₂-alkyl. In some embodiments, the alkyl is a C₁₋₅alkyl. In some embodiments, the alkyl is methyl, ethyl, or isopropyl. In some embodiments, the cycloalkyl is a C₃₋₆cycloalkyl. In some embodiments, the aryl is a phenyl. In some embodiments, the heterocyclyl is a 5- or 6-membered heterocyclyl having 1 or 2 heteroatoms selected from the group consisting of N, O, and S. In some embodiments, the heteroaryl is a 5- or 6-membered heteroaryl having 1, 2 or 3 heteroatoms selected from the group consisting of N, O, and S.

In some embodiments, R₇ is H, alkyl, cycloalkyl, —(C═O)—O-alkyl, —S(O)₂-alkyl. In some embodiments, R₇ is H, alkyl, cycloalkyl, —(C═O)—O-alkyl, or —S(O)₂-alkyl. In some embodiments, R₇ is H, alkyl, —(C═O)—O-alkyl, or —S(O)₂-alkyl. In some embodiments, R₇ is H, alkyl, or cycloalkyl. In some embodiments, R₇ is H or alkyl. In some embodiments, R₇ is alkyl. In some embodiments, the alkyl is a C₁₋₅alkyl. In some embodiments, the alkyl is methyl, ethyl, or isopropyl. In some embodiments, the alkyl is methyl. In some embodiments, the cycloalkyl is a C₃₋₆cycloalkyl.

In some embodiments, V is —O— or —CR₈R₉—. In some embodiments, V is —O— or —NR₁₀—. In some embodiments, V is —O—. In some embodiments, V is —CR₈R₉—. In some embodiments, R₈ and R₉ are each independently H or alkyl.

In some embodiments, X is —O— or —NR₁₃—. In some embodiments, X is —O— or —CR₁₁R₁₂—. In some embodiments, X is —CR₁₁R₁₂— or —NR₁₃—. In some embodiments, X is —CR₁₁R₁₂—.

In some embodiments, Y is a bond, —CR₈R₉—, or —NR₁₀—. In some embodiments, Y is a bond, —O—, or —CR₈R₉—. In some embodiments, Y is a bond or —CR₈R₉—. In some embodiments, Y is a bond. In some embodiments, Y is a —CR₈R₉—. In some embodiments, R₈ and R₉ together with the carbon atom to which they are attached form a C₃₋₆ cycloalkyl. In some embodiments, R₈ and R₉ together with the carbon atom to which they are attached form a cyclopropyl.

In some embodiments, Z is a —NR₁₀— or —CR₈R₉—. In some embodiments, Z is —NR₁₀—. In some embodiments, R₈ and R₉ are each independently H or alkyl. In some embodiments, R₈ and R₉ together with the carbon atom to which they are attached form a C₃₋₆ cycloalkyl. In some embodiments, R₈ and R₉ together with the carbon atom to which they are attached form a cyclopropyl. In some embodiments, R₁₀ is H, alkyl, —(C═O)alkyl, or —S(O)₂-alkyl. In some embodiments, R₁₀ is H or alkyl. In some embodiments, the alkyl is methyl, ethyl, or isopropyl. In some embodiments, the alkyl is methyl.

In some embodiments, V—(X)ₚ—Y—Z is —O—(CH₂)ₚ—CH₂—CR₈R₉—, —O—(CH₂)ₚCR₈R₉—, or —O—(CH₂)ₚ—CR₈R₉—O—, wherein p is 0, 1, or 2. In some embodiments, V—(X)ₚ—Y—Z is —O—(CH₂)ₚ—CH₂—CR₈R₉— or —O—(CH₂)ₚ—CR₈R₉—, wherein p is 0, 1, or 2. In some embodiments, V—(X)ₚ—Y—Z is —O—(CH₂)ₚ—CH₂—CR₈R₉— or —O—(CH₂)ₚ—CR₈R₉—, wherein p is 0 or 1. In some embodiments, V—(X)ₚ—Y—Z is —O—(CH₂)ₚ—CH₂—CH₂— or —O—(CH₂)ₚ—CH₂—, wherein p is 0 or 1. In some embodiments, V—(X)ₚ—Y—Z is —O—(CH₂)ₚ—CH₂—O—, wherein p is 1. In some embodiments, V—(X)ₚ—Y—Z is —O—CH₂—CH₂—CH₂—CH₂—, —O—CH₂—CH₂—CH₂—, —O—CH₂—CH₂—, —O—CH₂—, —O—CH (CH₃)—, —O—CH₂—CH₂—CH₂—O—, —O—CH₂—CH₂—O—. In some embodiments, V—(X)ₚ—Y—Z is —O—CH₂—CH₂—O—. In some embodiments, V—(X)ₚ—Y—Z is —O—CH₂—O—. In some embodiments, V—(X)ₚ—Y—Z is —O—CH₂—. In some embodiments, V—(X)ₚ—Y—Z is —O—CH₂—CH₂—.

In some embodiments, V—(X)ₚ—Y—Z does not comprise an —O—O— or —N—N— bond.

In some embodiments, R₈ and R₉ are each independently H, halogen, or alkyl. In some embodiments, R₈ and R₉ are each independently H or alkyl. In some embodiments, R₈ and R₉ are alkyl. In some embodiments, the alkyl is methyl or ethyl. In some embodiments, R₈ and R₉ are each independently H or halogen. In some embodiments, R₈ and R₉ are H. In some embodiments, R₈ and R₉ are halogen. In some embodiments, the halogen is fluoride. In some embodiments, R₈ and R₉ together with the carbon atom to which they are attached form a carbocycle or heterocycle. In some embodiments, the carbocycle is a C₃₋₆ cycloalkyl. In some embodiments, the carbocycle is a cyclopropyl. In some embodiments, the heterocycle is a 3- or 6-membered heterocycle. In some embodiments, the heterocycle comprises 1 or 2 heteroatoms selected from the group consisting of N, O, and S.

In some embodiments, R₁₀ is H, alkyl, cycloalkyl, —(C—O)—O-alkyl, —S(O)₂-alkyl. In some embodiments, R₁₀ is H, alkyl, cycloalkyl, —(C═O)—O-alkyl, or —S(O)₂-alkyl. In some embodiments, R₁₀ is H, alkyl, —(C═O)—O-alkyl, or —S(O)₂-alkyl. In some embodiments, R₁₀ is H, alkyl, or cycloalkyl. In some embodiments, R₁₀ is H or alkyl. In some embodiments, R₁₀ is alkyl. In some embodiments, the alkyl is a C₁₋₅alkyl. In some embodiments, the alkyl is methyl, ethyl, or isopropyl. In some embodiments, the alkyl is methyl. In some embodiments, the cycloalkyl is a C₃₋₆cycloalkyl.

In some embodiments, R₁₁ and R₁₂ are each independently H, halogen, or alkyl. In some embodiments, R₁₁ and R₁₂ are each independently H or alkyl. In some embodiments, R₁₁ and R₁₂ are alkyl. In some embodiments, the alkyl is methyl or ethyl. In some embodiments, R₁₁ and R₁₂ are each independently H or halogen. In some embodiments, R₁₁ and R₁₂ are H. In some embodiments, R₁₁ and R₁₂ are halogen. In some embodiments, the halogen is fluoride. In some embodiments, R₁₁ and R₁₂ together with the carbon atom to which they are attached form a carbocycle or heterocycle. In some embodiments, the carbocycle is a C₃₋₆ cycloalkyl. In some embodiments, the carbocycle is a cyclopropyl. In some embodiments, the heterocycle is a 3- or 6-membered heterocycle. In some embodiments, the heterocycle comprises 1 or 2 heteroatoms selected from the group consisting of N, O, and S.

In some embodiments, \textcircled{B} is phenyl or 5- or 6-membered heteroaryl, each of which is optionally substituted.

In some embodiments, \textcircled{B} is an optionally substituted phenyl. In some embodiments, the optionally substituted phenyl is:

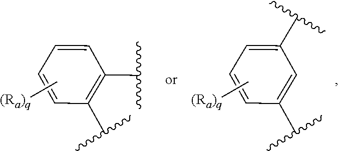

wherein Rₐ is halogen, alkyl, or alkoxy; and q is 0, 1, or 2. In some embodiments, the optionally substituted phenyl is:

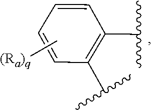

wherein Rₐ is halogen, alkyl, or alkoxy; and q is 0, 1, or 2. In some embodiments, the optionally substituted phenyl is:

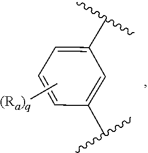

wherein Rₐ is halogen, alkyl, or alkoxy; and q is 0, 1, or 2. In some embodiments, Rₐ is halogen. In some embodiments, q is 0 or 1. In some embodiments, q is 0. In some embodiments, q is 1. In some embodiments, \textcircled{B} is

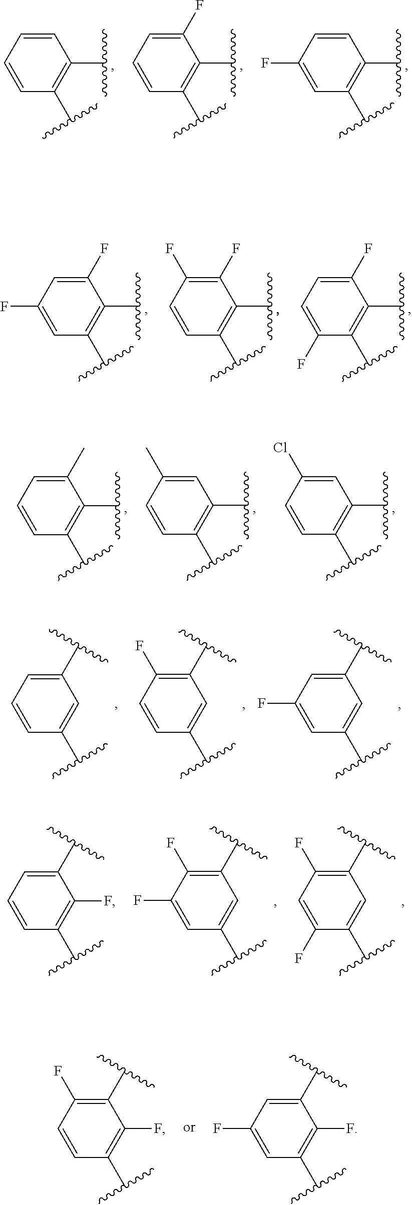

In some embodiments, \textcircled{B} is

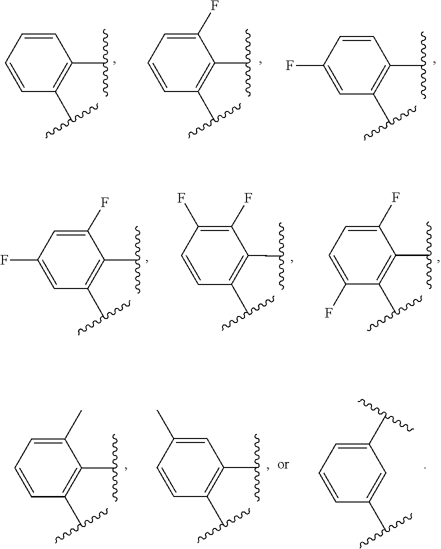

In some embodiments, \textcircled{B} is an optionally substituted 5-membered heteroaryl. In some embodiments, \textcircled{B} is optionally substituted 5-membered heteroaryl having 1, 2, or 3 heteroatoms selected from the group consisting of N, O, and S. In some embodiments, \textcircled{B} is optionally substituted 6-membered heteroaryl. In some embodiments, \textcircled{B} is optionally substituted 6-membered heteroaryl having 1, 2, or 3 heteroatoms selected from the group consisting of N, O, and S. In some embodiments, the optionally substituted 6-membered heteroaryl is selected from the group consisting of pyridinyl, pyrazinyl, pyrimidinyl, or pyridazinyl. In some embodiments, the optionally substituted 6-membered heteroaryl is:

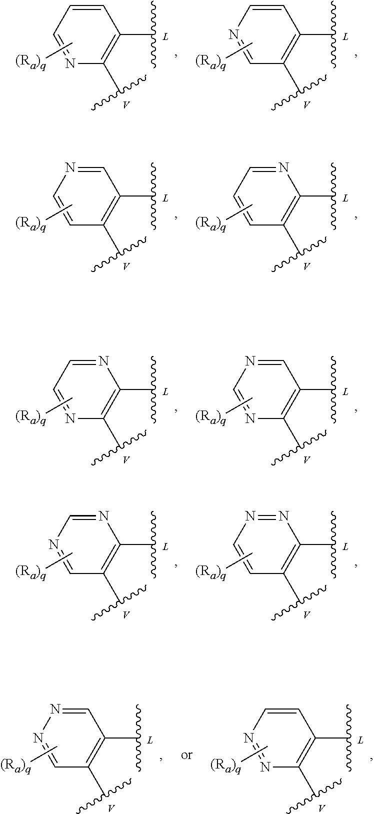

wherein Rₐ is halogen, alkyl, or alkoxy; and q is 0, 1, or 2. In some embodiments, the optionally substituted 6-membered heteroaryl is:

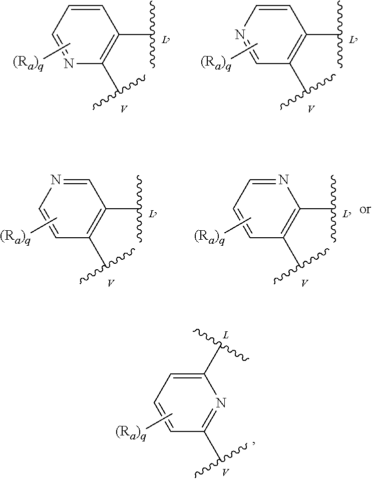

wherein Rₐ is halogen, alkyl, or alkoxy; and q is 0, 1, or 2. In some embodiments, Rₐ is halogen. In some embodiments, q is 0 or 1. In some embodiments, q is 0. In some embodiments, \textcircled{B} is

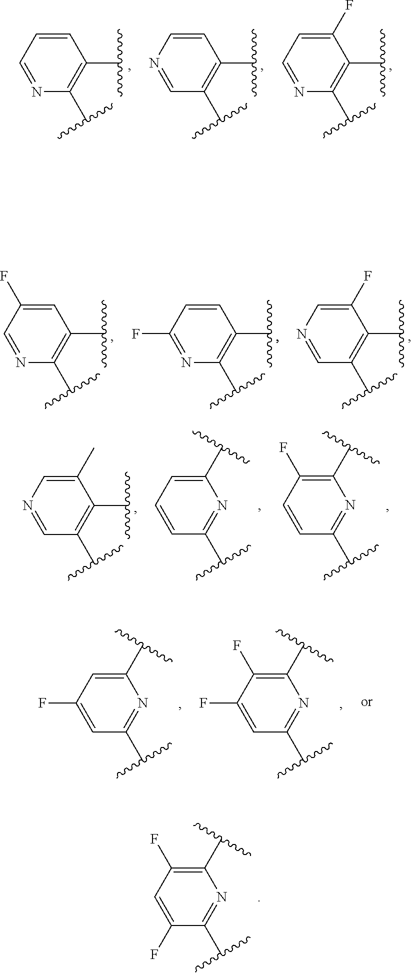

In some embodiments, \textcircled{B} is

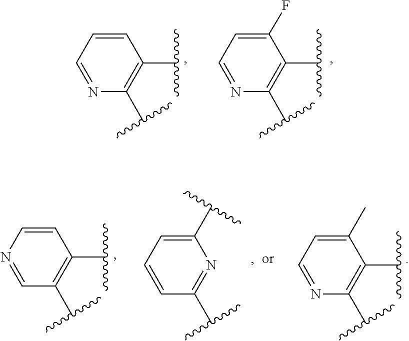

In some embodiments, \textcircled{B} is

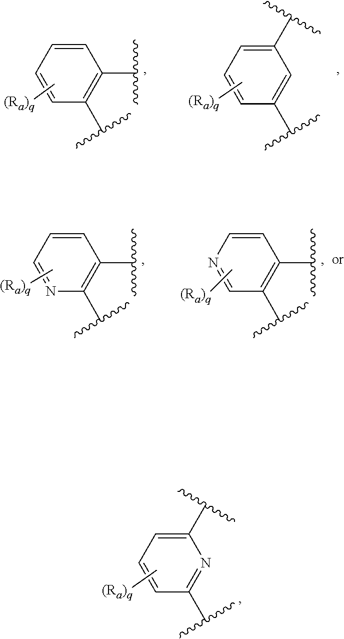

wherein Rₐ is halogen, alkyl, or alkoxy; and q is 0, 1, or 2

In some embodiments, \textcircled{B} is

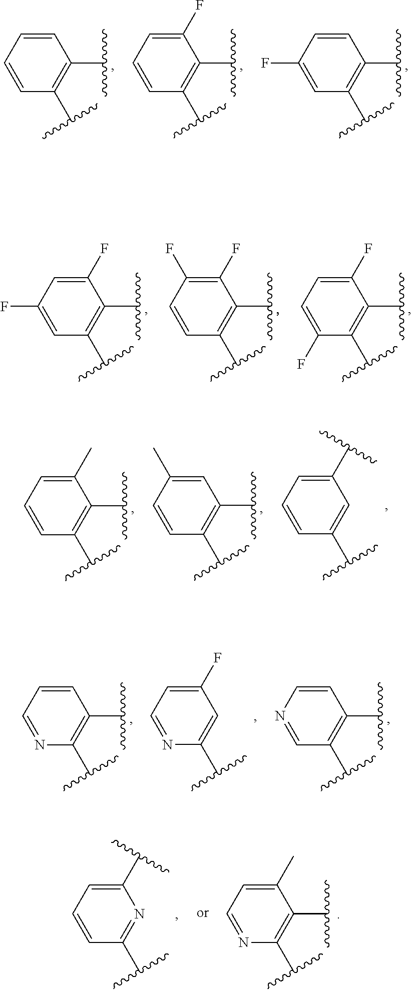

In some embodiments, m is 0 or 1. In some embodiments, m is 0. In some embodiments, m is 1

In some embodiments, n is 0 or 1. In some embodiments, n is 1. In some embodiments, n is 0.

In some embodiments, p is 0 or 1. In some embodiments, p is 0. In some embodiments, p is 1.

In some embodiments, L is a-carbocyclyl-O— or -heterocyclyl-O-linker having the structure

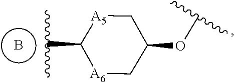

wherein A₅ and A₆ are each independently —O— or —CH₂—. In some embodiments, As is —O—. In some embodiments, A₅ is —CH₂—. In some embodiments, A₆ is —O—. In some embodiments, A₆ is —CH₂—.

In some embodiments, L is an aryl linker having the structure

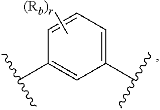

wherein R_{b} is halogen, alkyl, or alkoxy; and r is 0, 1, or 2. In some embodiments, R_{b} is halogen. In some embodiments, the halogen is fluoride. In some embodiments, r is 1. In some embodiments, r is 0. In some embodiments, the aryl linker is

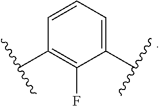 [SMILES: *c1cccc(*)c1F]

In some embodiments, L is a 5- or 6-membered heteroaryl linker. In some embodiments, L is a 5- or 6-membered heteroaryl linker having 1 or 2 nitrogen atoms. In some embodiments, linker L is a heteroaryl aryl linker having the structure

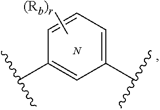

wherein R_{b} is halogen, alkyl, or alkoxy; and r is 0 or 1. In some embodiments, r is 0. In some embodiments, r is 1.

In some embodiments, A₁ is —C(O)— or —S(O)₂—, A₂ is —CR₅R₆—, A₃ is —O—, R₁ and R₂ are each independently H or alkyl, R₃ and R₄ are H, X is —CH₂—, V is —O—, Y is a bond or —CR₈R₉—, Z is —O— or —CR₈R₉—, m is 0 or 1, n is 0 or 1, and p is 0 or 1.

In some embodiments, A₁ is —C(O)— or —S(O)₂—, A₂ is —O—, A₃ is —CR₅R₆—, R₁ and R₂ are each independently H or alkyl, R₃ and R₄ are H, X is —CH₂—, V is —O—, Y is a bond or —CR₈R₉—, Z is —O— or —CR₈R₉—, m is 0 or 1, n is 0 or 1, and p is 0 or 1.

In some embodiments, A₁ is —C(O)—, A₃ is —O—, R₁ and R₂ are each independently H or alkyl, R₃ and R₄ are H, X is —CH₂—, V is —O—, Y is a bond or —CH₂—, Z is —O—, —CH₂—, or —N(alkyl)-, m is 0 or 1, n is 0 or 1, and p is 0 or 1.

In some embodiments, A₁ is —C(O)—, A₂ and A₄ are —CH₂—, A₃ is —O—, R₁ and R₂ are each independently H or alkyl, R₃ and R₄ are H, X is —CH₂—, V is —O—, Y is a bond or —CH₂—, Z is —O—, —CH₂—, —CH(Me)-, or —N(alkyl)-, m is 0 or 1, n is 0 or 1, and p is 0 or 1.

In some embodiments, A₁ is —C(O)—, A₂ and A₄ are —CH₂—, A₃ is —O—, L is -carbocyclyl-O— or -heterocyclyl-O—, R₁ and R₂ are each independently H or alkyl, R₃ and R₄ are H, X is —CH₂—, V is —O—, Y is a bond or —CH₂—, Z is —O—, —CH₂—, —CH(Me)-, or —N(alkyl)-, m is 0 or 1, n is 0 or 1, and p is 0 or 1.

In some embodiments, A₁ is —C(O)—, A₂ and A₄ are —CH₂—, A₃ is —O—, L is

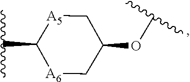

wherein A₅ and A₆ are each independently —CH₂— or —O—, R₁ and R₂ are each independently H or alkyl, R₃ and R₄ are H, X is —CH₂—, V is —O—, Y is a bond or —CH₂—, Z is —O—, —CH₂—, —CH(Me)-, or —N(alkyl)-, m is 0 or 1, n is 0 or 1, and p is 0 or 1.

In some embodiments, A₁ is —C(O)—, A₂ is —O—, A₃ and A₄ are —CH₂—, L is

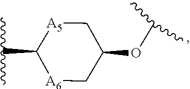

wherein A₅ and A₆ are each independently —CH₂— or —O—, R₁ and R₂ are each independently H or alkyl, R₃ and R₄ are H, X is —CH₂—, V is —O—, Y is a bond or —CH₂—, Z is —O—, —CH₂—, —CH(Me)-, or —N(alkyl)-, m is 0 or 1, n is 0 or 1, and p is 0 or 1.

In some embodiments, A₁ is —C(O)—, A₂ and A₄ are —CH₂—, A₃ is —O—, \textcircled{B} is optionally substituted phenyl or optionally substituted heteroaryl having 1 or 2 N atoms, L is

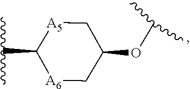

wherein A₅ and A₆ are each independently —CH₂— or —O—, R₁ and R₂ are each independently H or alkyl, R₃ and R₄ are H, X is —CH₂—, V is —O—, Y is a bond or —CH₂—, Z is —O—, —CH₂—, —CH(Me)-, or —N(alkyl)-, m is 0 or 1, n is 0 or 1, and p is 0 or 1.

In some embodiments, A₁ is —C(O)—, A₂ is —O—, A₃ and A₄ are —CH₂—, \textcircled{B} is optionally substituted phenyl or optionally substituted heteroaryl having 1 or 2 N atoms, L is

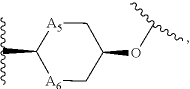

wherein A₅ and A₆ are each independently —CH₂— or —O—, R₁ and R₂ are each independently H or alkyl, R₃ and R₄ are H, X is —CH₂—, V is —O—, Y is a bond or —CH₂—, Z is —O—, —CH₂—, —CH(Me)-, or —N(alkyl)-, m is 0 or 1, n is 0 or 1, and p is 0 or 1.

In some embodiments, A₁ is —C(O)—, A₂ and A₄ are —CH₂—, A₃ is —O—, \textcircled{B} is optionally substituted phenyl or optionally substituted heteroaryl having 1 or 2 N atoms, L is

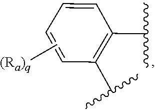

wherein Rₐ is halogen, alkyl, or alkoxy and q is 0 or 1, R₁ and R₂ are each independently H or alkyl, R₃ and R₄ are H, X is —CH₂—, V is —O—, Y is a bond or —CH₂—, Z is —O—, —CH₂—, —CH(Me)-, or —N(alkyl)-, m is 0 or 1, n is 0 or 1, and p is 0 or 1.

In some embodiments, A₁ is —C(O)—, A₂ and A₄ are —CH₂—, A₃ is —O—, \textcircled{B} is optionally substituted phenyl, L is

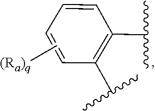

wherein Rₐ is halogen, alkyl, or alkoxy and q is 0 or 1, R₁ and R₂ are each independently H or alkyl, R₃ and R₄ are H, X is —CH₂—, V is —O—, Y is a bond or —CH₂—, Z is —O—, —CH₂—, —CH(Me)-, or —N(alkyl)-, m is 0 or 1, n is 0 or 1, and p is 0 or 1.

In some embodiments, the compound of the present disclosure has one of the following structures:

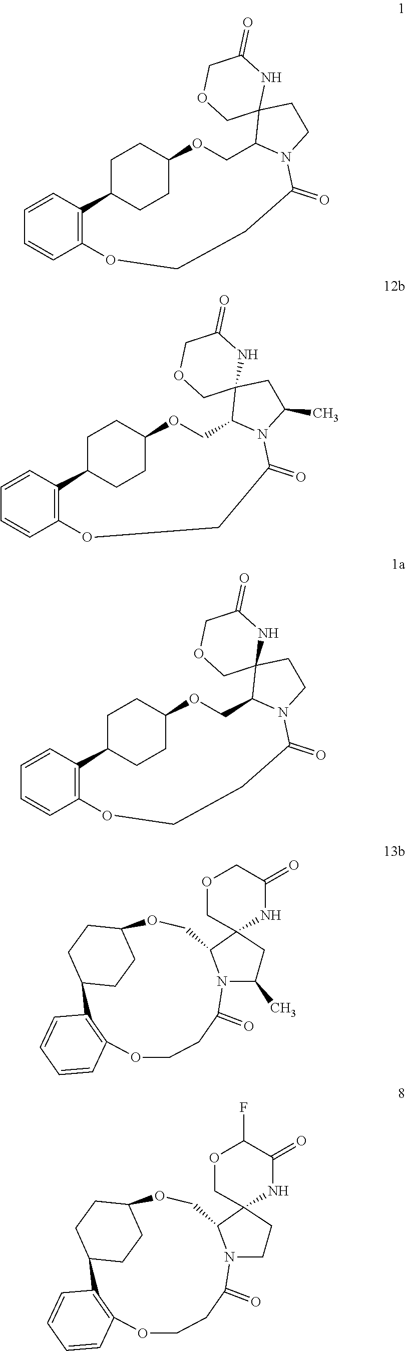

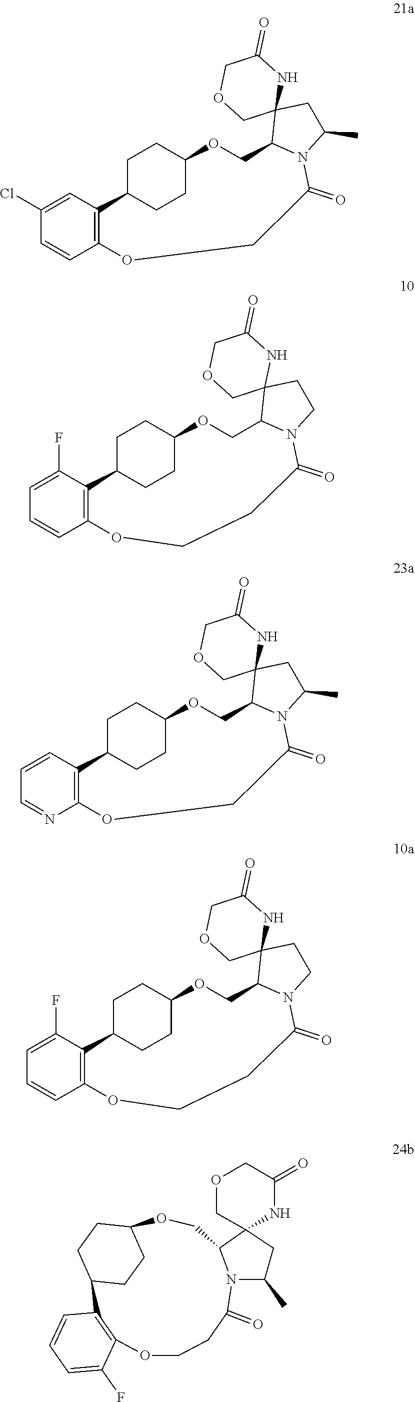 [SMILES: C[C@@H]1C[C@@]2(COCC(=O)N2)[C@@H]2CO[C@H]3CC[C@H](CC3)c3cc(Cl)ccc3OCC(=O)N12 ; O=C1COCC2(CCN3C(=O)CCOc4cccc(F)c4[C@H]4CC[C@H](CC4)OCC32)N1 ; C[C@@H]1C[C@@]2(COCC(=O)N2)[C@@H]2CO[C@H]3CC[C@H](CC3)c3cccnc3OCC(=O)N12 ; O=C1COC[C@@]2(CCN3C(=O)CCOc4cccc(F)c4[C@H]4CC[C@H](CC4)OC[C@H]32)N1 ; C[C@@H]1C[C@]2(COCC(=O)N2)[C@H]2CO[C@H]3CC[C@H](CC3)c3cccc(F)c3OCCC(=O)N12]

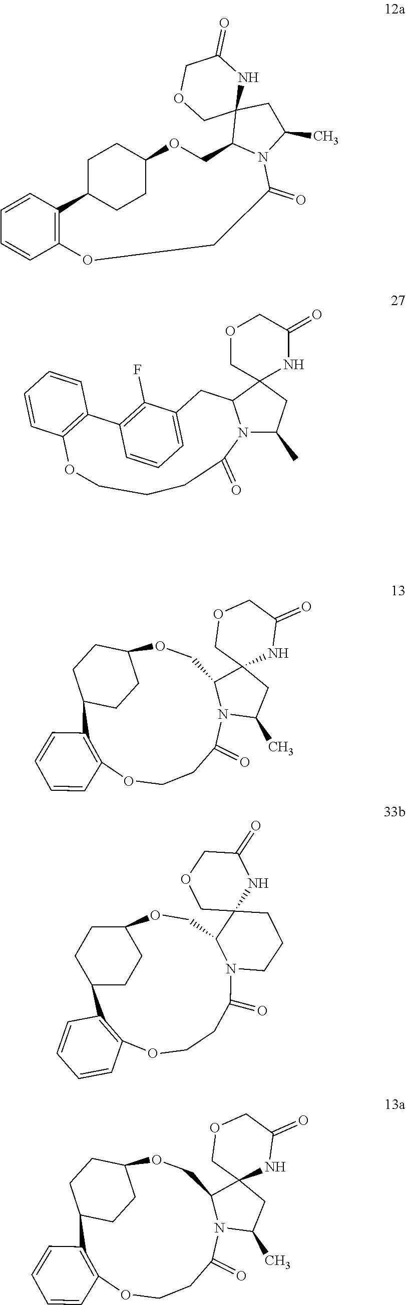 [SMILES: C[C@@H]1C[C@@]2(COCC(=O)N2)[C@@H]2CO[C@H]3CC[C@H](CC3)c3ccccc3OCC(=O)N12 ; C[C@@H]1CC2(COCC(=O)N2)C2Cc3cccc(c3F)-c3ccccc3OCCCC(=O)N21 ; C[C@@H]1C[C@]2(COCC(=O)N2)[C@H]2CO[C@H]3CC[C@H](CC3)c3ccccc3OCCC(=O)N12 ; O=C1COC[C@]2(CCCN3C(=O)CCOc4ccccc4[C@H]4CC[C@H](CC4)OC[C@@H]32)N1 ; C[C@@H]1C[C@@]2(COCC(=O)N2)[C@@H]2CO[C@H]3CC[C@H](CC3)c3ccccc3OCCC(=O)N12]

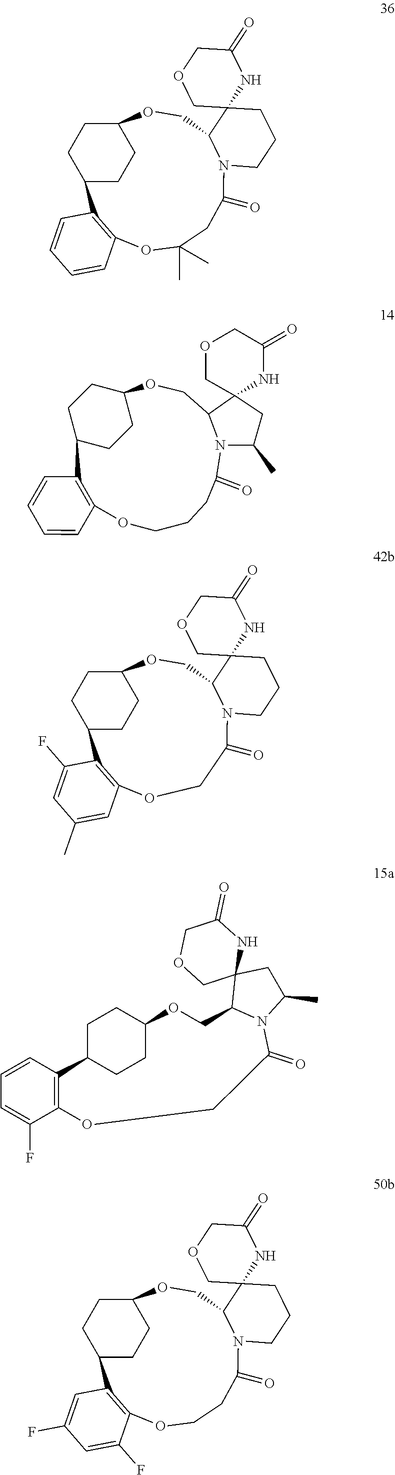 [SMILES: CC1(C)CC(=O)N2CCC[C@]3(COCC(=O)N3)[C@H]2CO[C@H]2CC[C@H](CC2)c2ccccc2O1 ; C[C@@H]1C[C@]2(COCC(=O)N2)C2CO[C@H]3CC[C@H](CC3)c3ccccc3OCCCC(=O)N21 ; Cc1cc(F)c2c(c1)OCC(=O)N1CCC[C@]3(COCC(=O)N3)[C@H]1CO[C@H]1CC[C@@H]2CC1 ; C[C@@H]1C[C@@]2(COCC(=O)N2)[C@@H]2CO[C@H]3CC[C@H](CC3)c3cccc(F)c3OCC(=O)N12 ; O=C1COC[C@]2(CCCN3C(=O)CCOc4c(F)cc(F)cc4[C@H]4CC[C@H](CC4)OC[C@@H]32)N1]

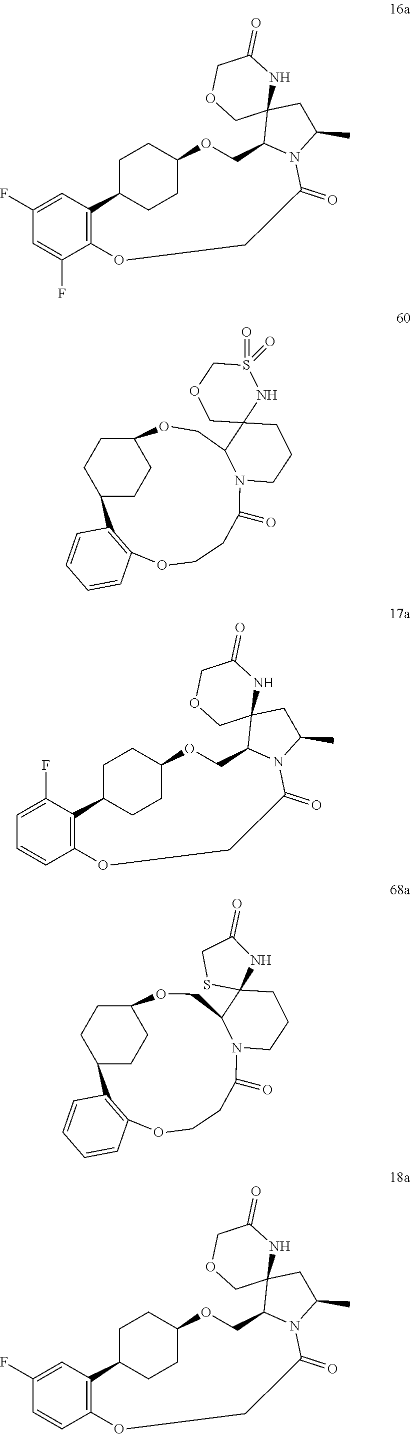 [SMILES: C[C@@H]1C[C@@]2(COCC(=O)N2)[C@@H]2CO[C@H]3CC[C@H](CC3)c3cc(F)cc(F)c3OCC(=O)N12 ; O=C1CCOc2ccccc2[C@H]2CC[C@H](CC2)OCC2N1CCCC21COCS(=O)(=O)N1 ; C[C@@H]1C[C@@]2(COCC(=O)N2)[C@@H]2CO[C@H]3CC[C@H](CC3)c3c(F)cccc3OCC(=O)N12 ; O=C1CS[C@@]2(CCCN3C(=O)CCOc4ccccc4[C@H]4CC[C@H](CC4)OC[C@H]32)N1 ; C[C@@H]1C[C@@]2(COCC(=O)N2)[C@@H]2CO[C@H]3CC[C@H](CC3)c3cc(F)ccc3OCC(=O)N12]

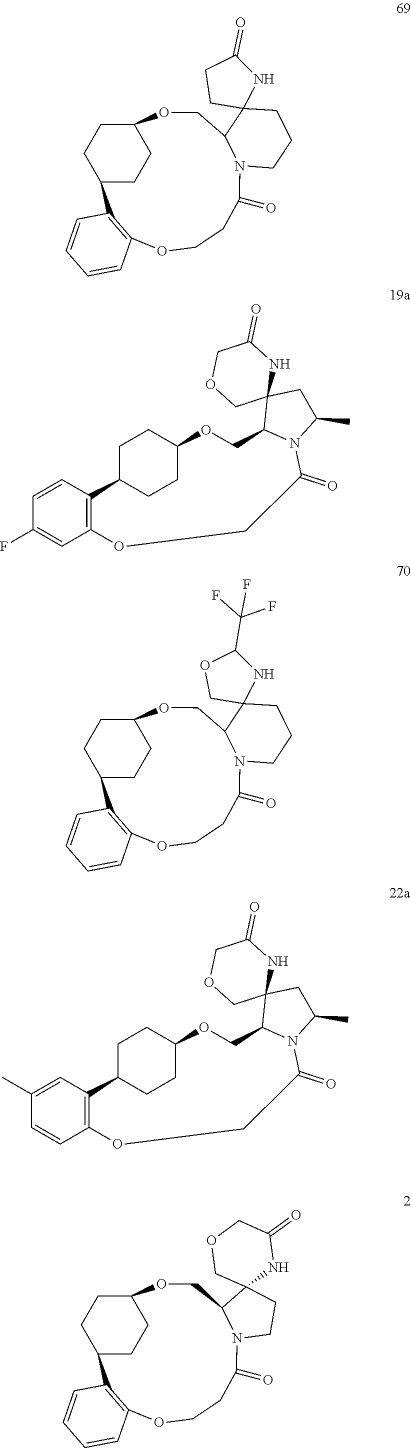

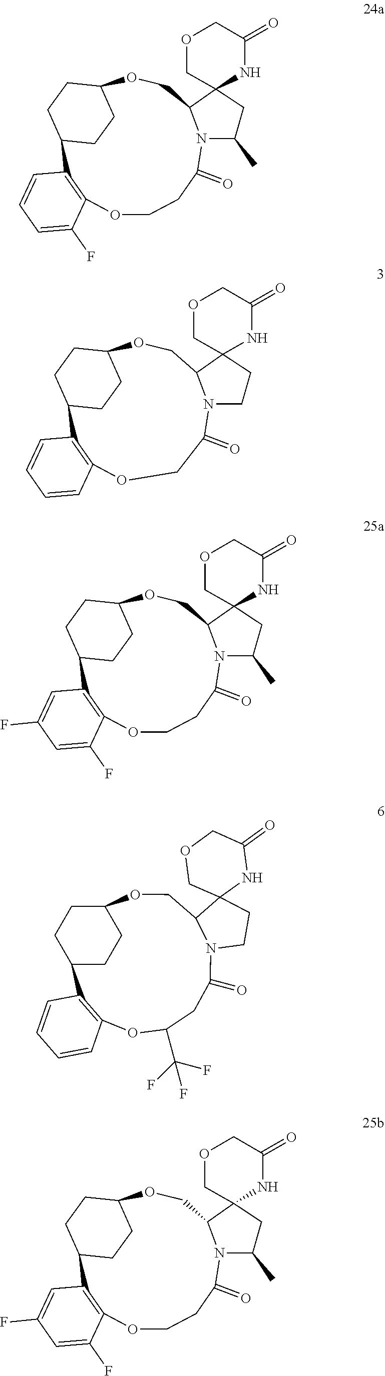 [SMILES: C[C@@H]1C[C@@]2(COCC(=O)N2)[C@@H]2CO[C@H]3CC[C@H](CC3)c3cccc(F)c3OCCC(=O)N12 ; O=C1COCC2(CCN3C(=O)COc4ccccc4[C@H]4CC[C@H](CC4)OCC32)N1 ; C[C@@H]1C[C@@]2(COCC(=O)N2)[C@@H]2CO[C@H]3CC[C@H](CC3)c3cc(F)cc(F)c3OCCC(=O)N12 ; O=C1COCC2(CCN3C(=O)CC(C(F)(F)F)Oc4ccccc4[C@H]4CC[C@H](CC4)OCC32)N1 ; C[C@@H]1C[C@]2(COCC(=O)N2)[C@H]2CO[C@H]3CC[C@H](CC3)c3cc(F)cc(F)c3OCCC(=O)N12]

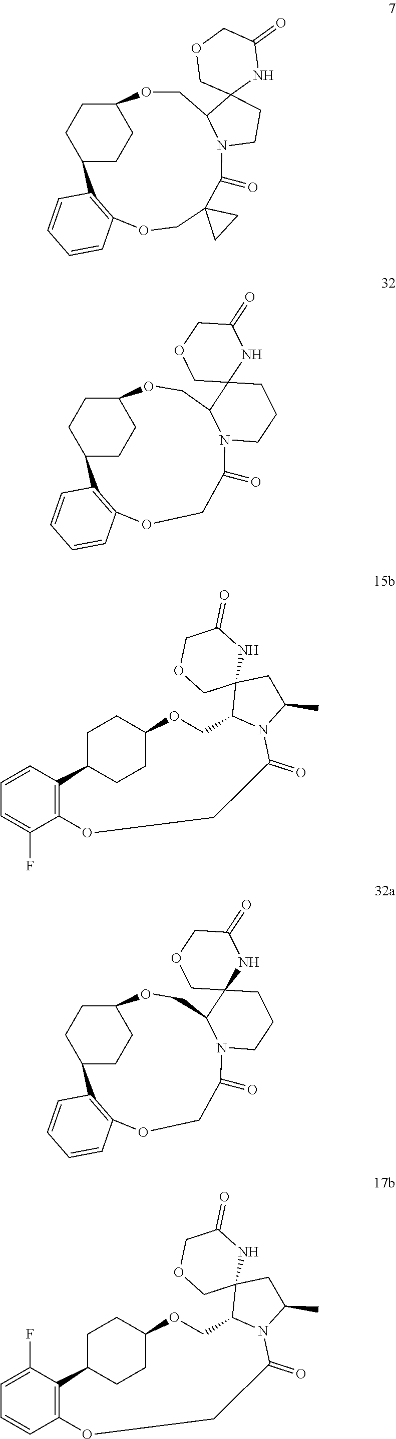 [SMILES: O=C1COCC2(CCN3C(=O)C4(CC4)COc4ccccc4[C@H]4CC[C@H](CC4)OCC32)N1 ; O=C1COCC2(CCCN3C(=O)COc4ccccc4[C@H]4CC[C@H](CC4)OCC32)N1 ; C[C@@H]1C[C@]2(COCC(=O)N2)[C@H]2CO[C@H]3CC[C@H](CC3)c3cccc(F)c3OCC(=O)N12 ; O=C1COC[C@@]2(CCCN3C(=O)COc4ccccc4[C@H]4CC[C@H](CC4)OC[C@H]32)N1 ; C[C@@H]1C[C@]2(COCC(=O)N2)[C@H]2CO[C@H]3CC[C@H](CC3)c3c(F)cccc3OCC(=O)N12]

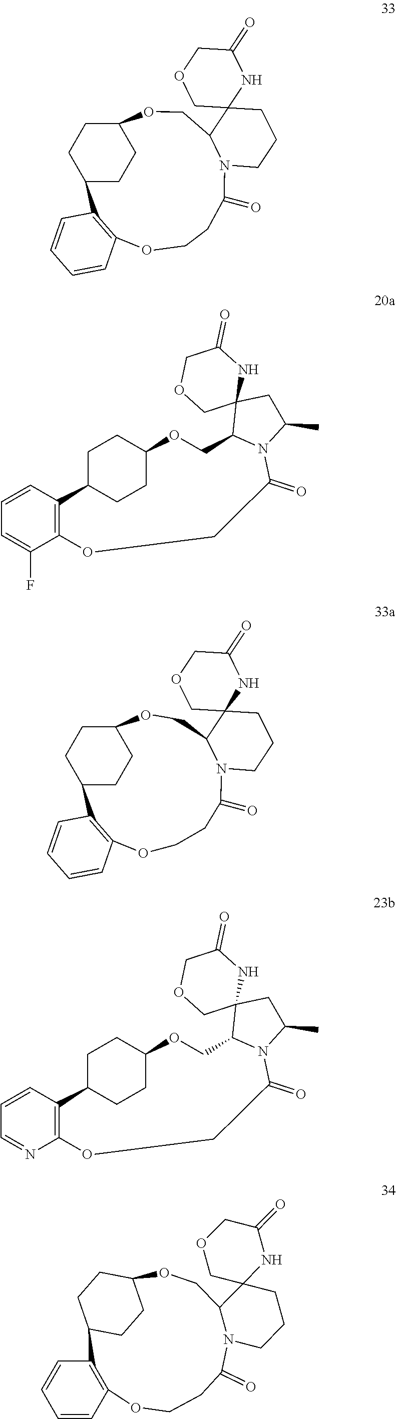 [SMILES: O=C1COCC2(CCCN3C(=O)CCOc4ccccc4[C@H]4CC[C@H](CC4)OCC32)N1 ; C[C@@H]1C[C@@]2(COCC(=O)N2)[C@@H]2CO[C@H]3CC[C@H](CC3)c3cccc(F)c3OCC(=O)N12 ; O=C1COC[C@@]2(CCCN3C(=O)CCOc4ccccc4[C@H]4CC[C@H](CC4)OC[C@H]32)N1 ; C[C@@H]1C[C@]2(COCC(=O)N2)[C@H]2CO[C@H]3CC[C@H](CC3)c3cccnc3OCC(=O)N12 ; O=C1COCC2(CCCN3C(=O)CCOc4ccccc4[C@H]4CC[C@H](CC4)OCC32)N1]

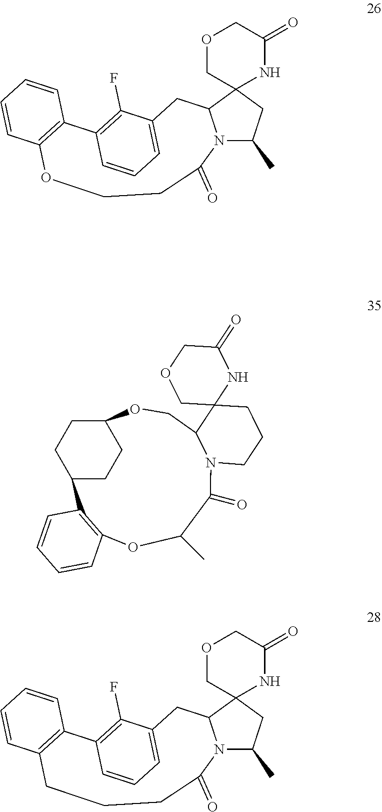 [SMILES: C[C@@H]1CC2(COCC(=O)N2)C2Cc3cccc(c3F)-c3ccccc3OCCC(=O)N21 ; CC1Oc2ccccc2[C@H]2CC[C@H](CC2)OCC2N(CCCC23COCC(=O)N3)C1=O ; C[C@@H]1CC2(COCC(=O)N2)C2Cc3cccc(c3F)-c3ccccc3CCCC(=O)N21]

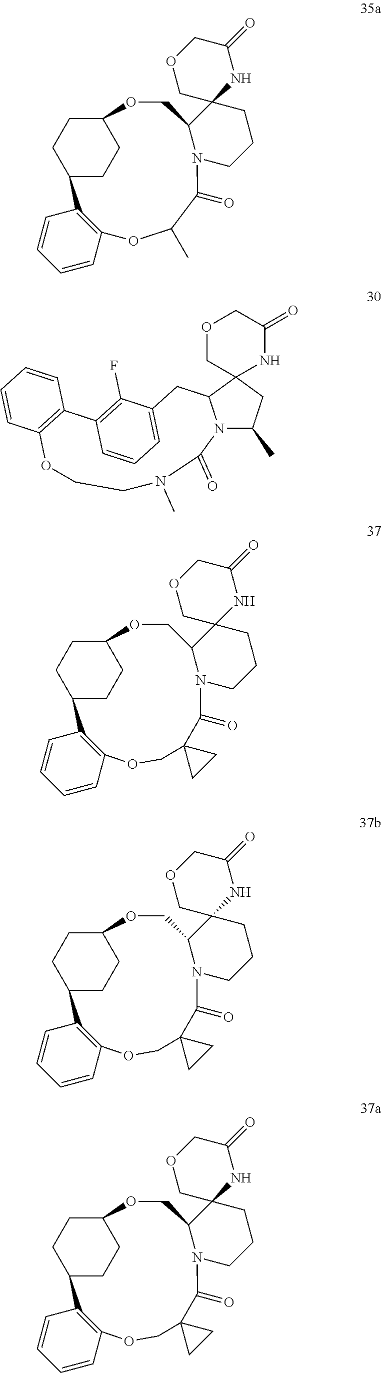 [SMILES: CC1Oc2ccccc2[C@H]2CC[C@H](CC2)OC[C@@H]2N(CCC[C@@]23COCC(=O)N3)C1=O ; C[C@@H]1CC2(COCC(=O)N2)C2Cc3cccc(c3F)-c3ccccc3OCCN(C)C(=O)N21 ; O=C1COCC2(CCCN3C(=O)C4(CC4)COc4ccccc4[C@H]4CC[C@H](CC4)OCC32)N1 ; O=C1COC[C@]2(CCCN3C(=O)C4(CC4)COc4ccccc4[C@H]4CC[C@H](CC4)OC[C@@H]32)N1 ; O=C1COC[C@@]2(CCCN3C(=O)C4(CC4)COc4ccccc4[C@H]4CC[C@H](CC4)OC[C@H]32)N1]

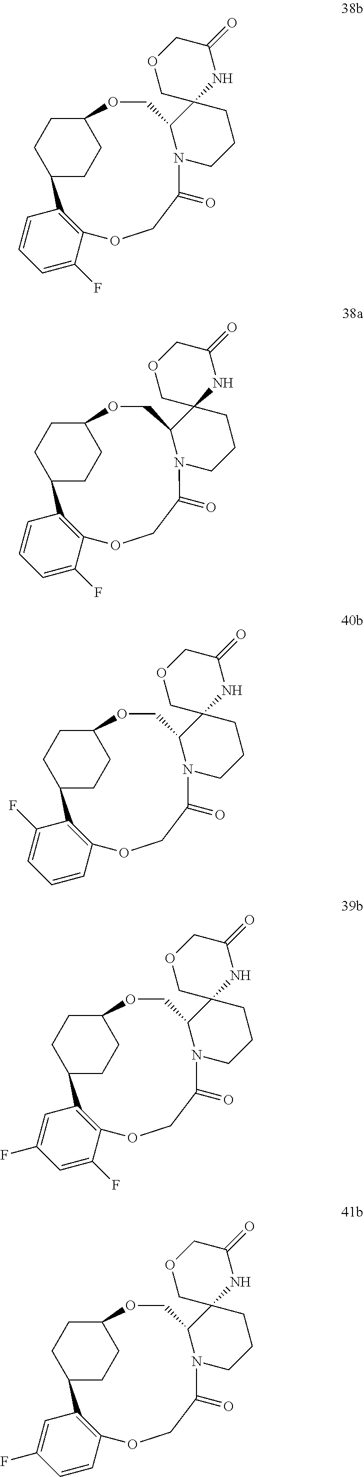 [SMILES: O=C1COC[C@]2(CCCN3C(=O)COc4c(F)cccc4[C@H]4CC[C@H](CC4)OC[C@@H]32)N1 ; O=C1COC[C@@]2(CCCN3C(=O)COc4c(F)cccc4[C@H]4CC[C@H](CC4)OC[C@H]32)N1 ; O=C1COC[C@]2(CCCN3C(=O)COc4cccc(F)c4[C@H]4CC[C@H](CC4)OC[C@@H]32)N1 ; O=C1COC[C@]2(CCCN3C(=O)COc4c(F)cc(F)cc4[C@H]4CC[C@H](CC4)OC[C@@H]32)N1 ; O=C1COC[C@]2(CCCN3C(=O)COc4ccc(F)cc4[C@H]4CC[C@H](CC4)OC[C@@H]32)N1]

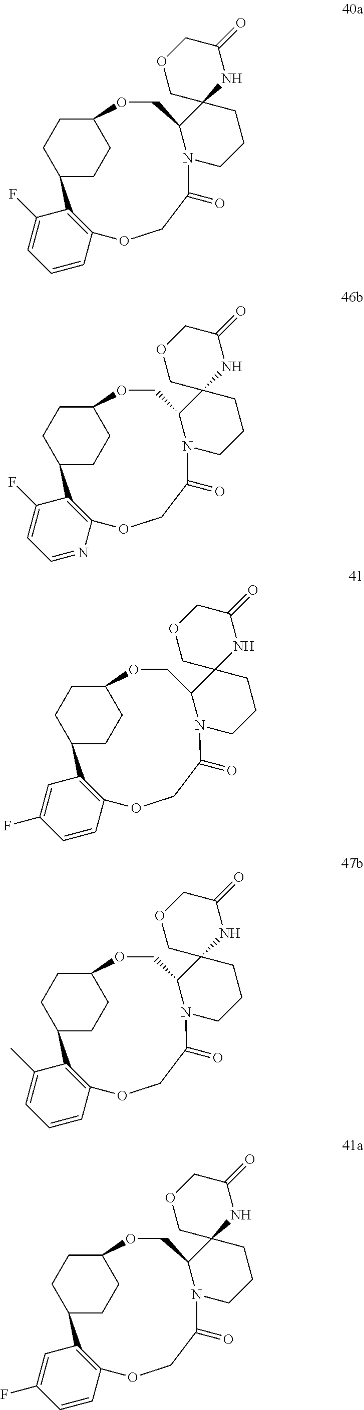 [SMILES: O=C1COC[C@@]2(CCCN3C(=O)COc4cccc(F)c4[C@H]4CC[C@H](CC4)OC[C@H]32)N1 ; O=C1COC[C@]2(CCCN3C(=O)COc4nccc(F)c4[C@H]4CC[C@H](CC4)OC[C@@H]32)N1 ; O=C1COCC2(CCCN3C(=O)COc4ccc(F)cc4[C@H]4CC[C@H](CC4)OCC32)N1 ; Cc1cccc2c1[C@H]1CC[C@H](CC1)OC[C@H]1N(CCC[C@]13COCC(=O)N3)C(=O)CO2 ; O=C1COC[C@@]2(CCCN3C(=O)COc4ccc(F)cc4[C@H]4CC[C@H](CC4)OC[C@H]32)N1]

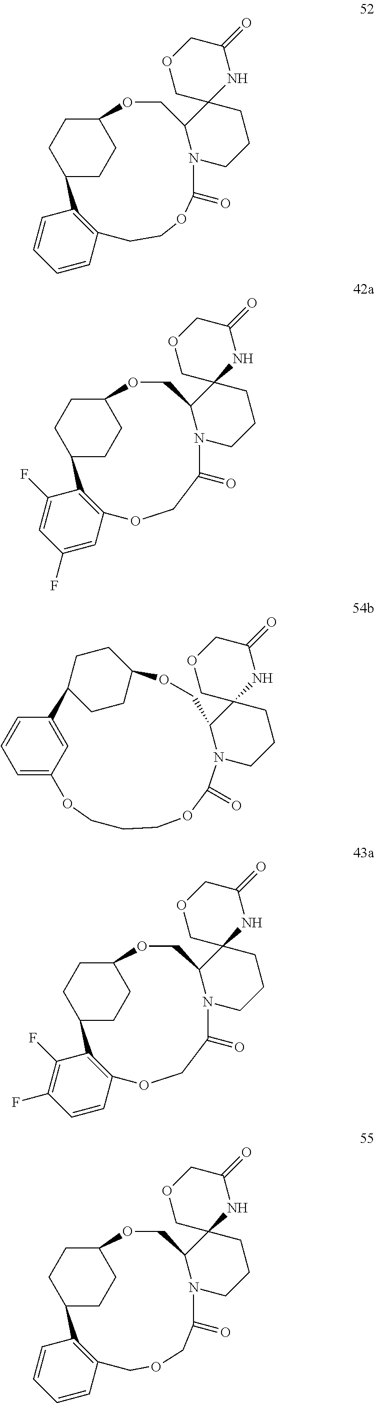 [SMILES: O=C1COCC2(CCCN3C(=O)OCCc4ccccc4[C@H]4CC[C@H](CC4)OCC32)N1 ; O=C1COC[C@@]2(CCCN3C(=O)COc4cc(F)cc(F)c4[C@H]4CC[C@H](CC4)OC[C@H]32)N1 ; O=C1COC[C@]2(CCCN3C(=O)OCCCOc4cccc(c4)[C@H]4CC[C@H](CC4)OC[C@@H]32)N1 ; O=C1COC[C@@]2(CCCN3C(=O)COc4ccc(F)c(F)c4[C@H]4CC[C@H](CC4)OC[C@H]32)N1 ; O=C1COC[C@@]2(CCCN3C(=O)COCc4ccccc4[C@H]4CC[C@H](CC4)OC[C@H]32)N1]

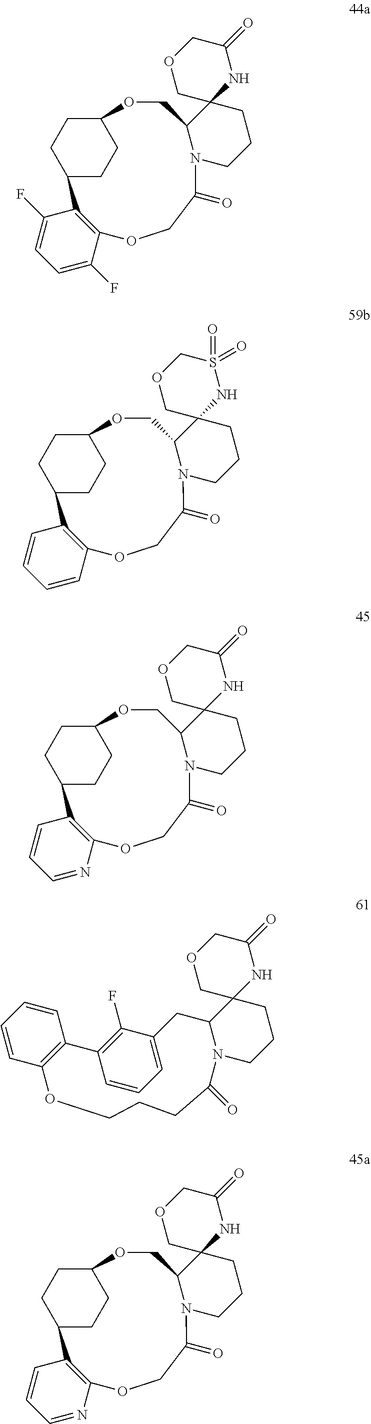 [SMILES: O=C1COC[C@@]2(CCCN3C(=O)COc4c(F)ccc(F)c4[C@H]4CC[C@H](CC4)OC[C@H]32)N1 ; O=C1COc2ccccc2[C@H]2CC[C@H](CC2)OC[C@H]2N1CCC[C@]21COCS(=O)(=O)N1 ; O=C1COCC2(CCCN3C(=O)COc4ncccc4[C@H]4CC[C@H](CC4)OCC32)N1 ; O=C1COCC2(CCCN3C(=O)CCCOc4ccccc4-c4cccc(c4F)CC32)N1 ; O=C1COC[C@@]2(CCCN3C(=O)COc4ncccc4[C@H]4CC[C@H](CC4)OC[C@H]32)N1]

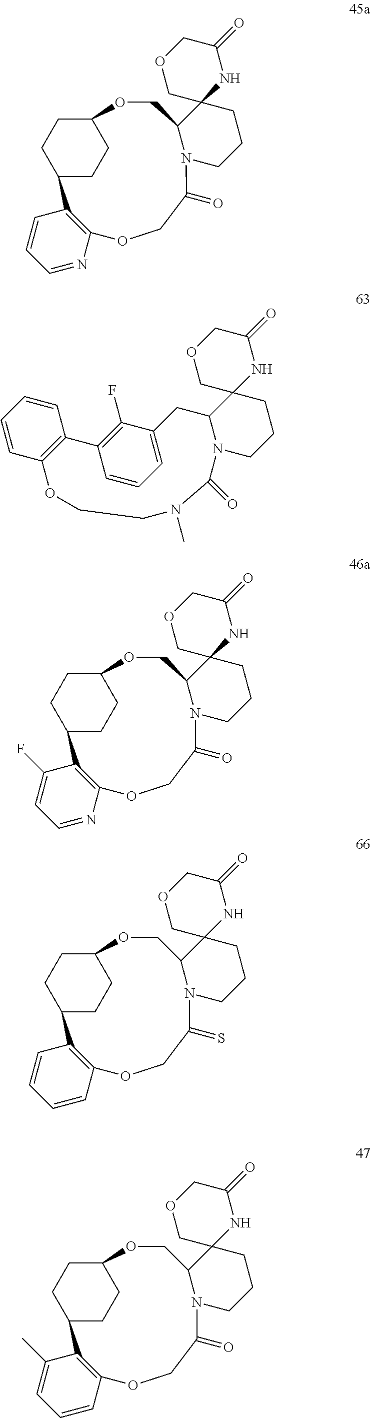 [SMILES: O=C1COC[C@@]2(CCCN3C(=O)COc4ncccc4[C@H]4CC[C@H](CC4)OC[C@H]32)N1 ; CN1CCOc2ccccc2-c2cccc(c2F)CC2N(CCCC23COCC(=O)N3)C1=O ; O=C1COC[C@@]2(CCCN3C(=O)COc4nccc(F)c4[C@H]4CC[C@H](CC4)OC[C@H]32)N1 ; O=C1COCC2(CCCN3C(=S)COc4ccccc4[C@H]4CC[C@H](CC4)OCC32)N1 ; Cc1cccc2c1[C@H]1CC[C@H](CC1)OCC1N(CCCC13COCC(=O)N3)C(=O)CO2]

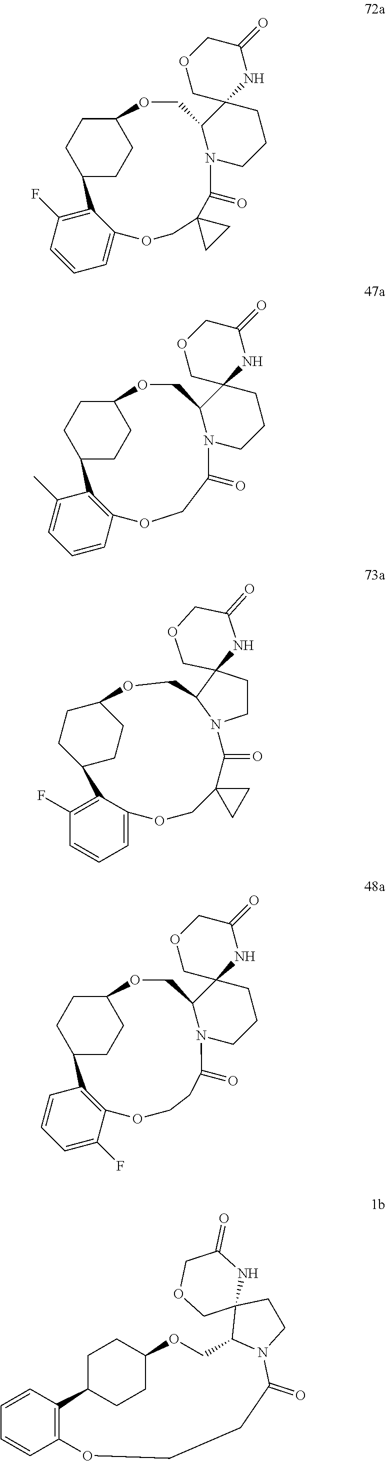 [SMILES: O=C1COC[C@]2(CCCN3C(=O)C4(CC4)COc4cccc(F)c4[C@H]4CC[C@H](CC4)OC[C@@H]32)N1 ; Cc1cccc2c1[C@H]1CC[C@H](CC1)OC[C@@H]1N(CCC[C@@]13COCC(=O)N3)C(=O)CO2 ; O=C1COC[C@@]2(CCN3C(=O)C4(CC4)COc4cccc(F)c4[C@H]4CC[C@H](CC4)OC[C@H]32)N1 ; O=C1COC[C@@]2(CCCN3C(=O)CCOc4c(F)cccc4[C@H]4CC[C@H](CC4)OC[C@H]32)N1 ; O=C1COC[C@]2(CCN3C(=O)CCOc4ccccc4[C@H]4CC[C@H](CC4)OC[C@@H]32)N1]

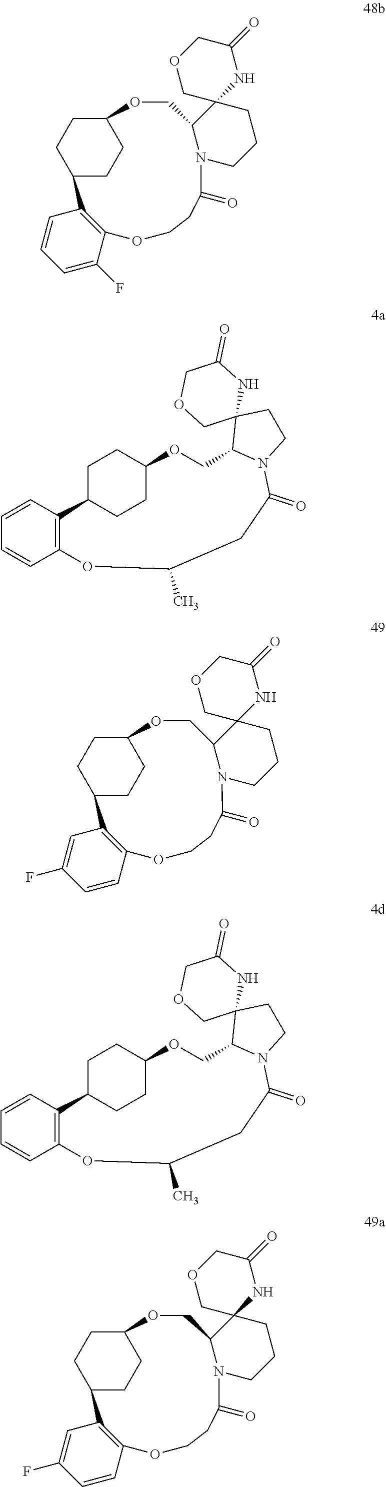 [SMILES: O=C1COC[C@]2(CCCN3C(=O)CCOc4c(F)cccc4[C@H]4CC[C@H](CC4)OC[C@@H]32)N1 ; C[C@H]1CC(=O)N2CC[C@]3(COCC(=O)N3)[C@H]2CO[C@H]2CC[C@H](CC2)c2ccccc2O1 ; O=C1COCC2(CCCN3C(=O)CCOc4ccc(F)cc4[C@H]4CC[C@H](CC4)OCC32)N1 ; C[C@@H]1CC(=O)N2CC[C@]3(COCC(=O)N3)[C@H]2CO[C@H]2CC[C@H](CC2)c2ccccc2O1 ; O=C1COC[C@@]2(CCCN3C(=O)CCOc4ccc(F)cc4[C@H]4CC[C@H](CC4)OC[C@H]32)N1]

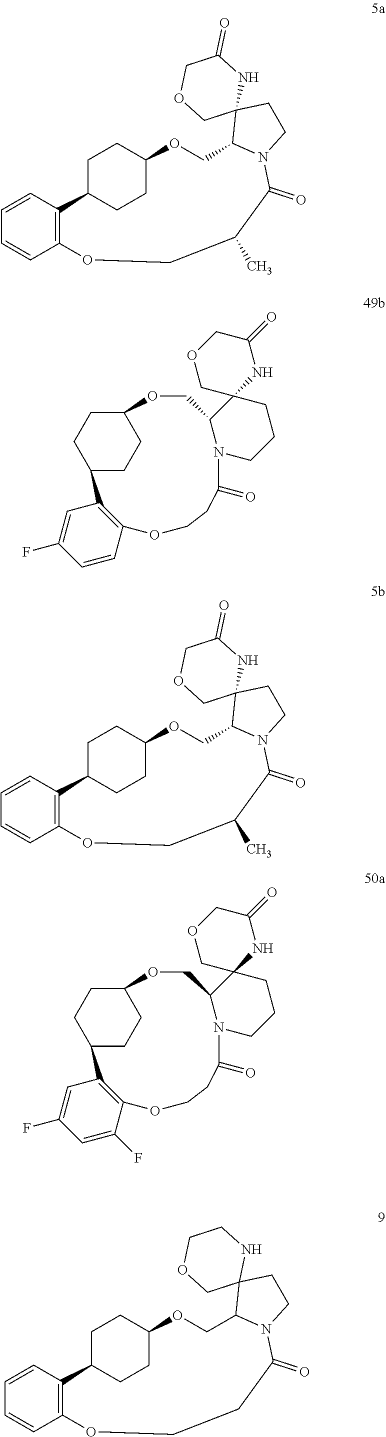 [SMILES: C[C@@H]1COc2ccccc2[C@H]2CC[C@H](CC2)OC[C@H]2N(CC[C@]23COCC(=O)N3)C1=O ; O=C1COC[C@]2(CCCN3C(=O)CCOc4ccc(F)cc4[C@H]4CC[C@H](CC4)OC[C@@H]32)N1 ; C[C@H]1COc2ccccc2[C@H]2CC[C@H](CC2)OC[C@H]2N(CC[C@]23COCC(=O)N3)C1=O ; O=C1COC[C@@]2(CCCN3C(=O)CCOc4c(F)cc(F)cc4[C@H]4CC[C@H](CC4)OC[C@H]32)N1 ; O=C1CCOc2ccccc2[C@H]2CC[C@H](CC2)OCC2N1CCC21COCCN1]

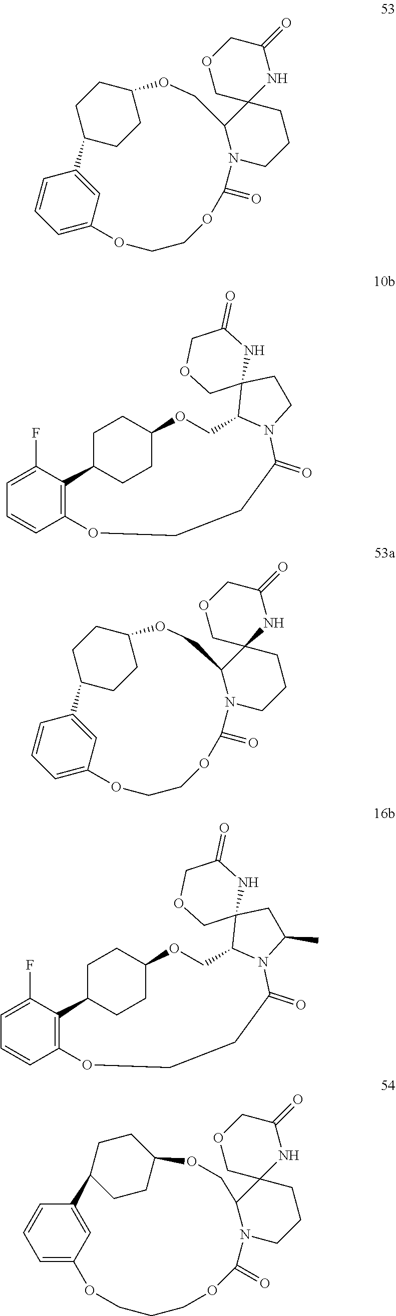 [SMILES: O=C1COCC2(CCCN3C(=O)OCCOc4cccc(c4)[C@H]4CC[C@H](CC4)OCC32)N1 ; O=C1COC[C@]2(CCN3C(=O)CCOc4cccc(F)c4[C@H]4CC[C@H](CC4)OC[C@@H]32)N1 ; O=C1COC[C@@]2(CCCN3C(=O)OCCOc4cccc(c4)[C@H]4CC[C@H](CC4)OC[C@H]32)N1 ; C[C@@H]1C[C@]2(COCC(=O)N2)[C@H]2CO[C@H]3CC[C@H](CC3)c3c(F)cccc3OCCC(=O)N12 ; O=C1COCC2(CCCN3C(=O)OCCCOc4cccc(c4)[C@H]4CC[C@H](CC4)OCC32)N1]

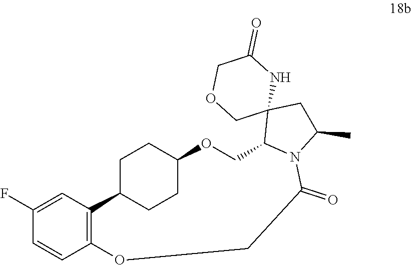 [SMILES: C[C@@H]1C[C@]2(COCC(=O)N2)[C@H]2CO[C@H]3CC[C@H](CC3)c3cc(F)ccc3OCC(=O)N12]

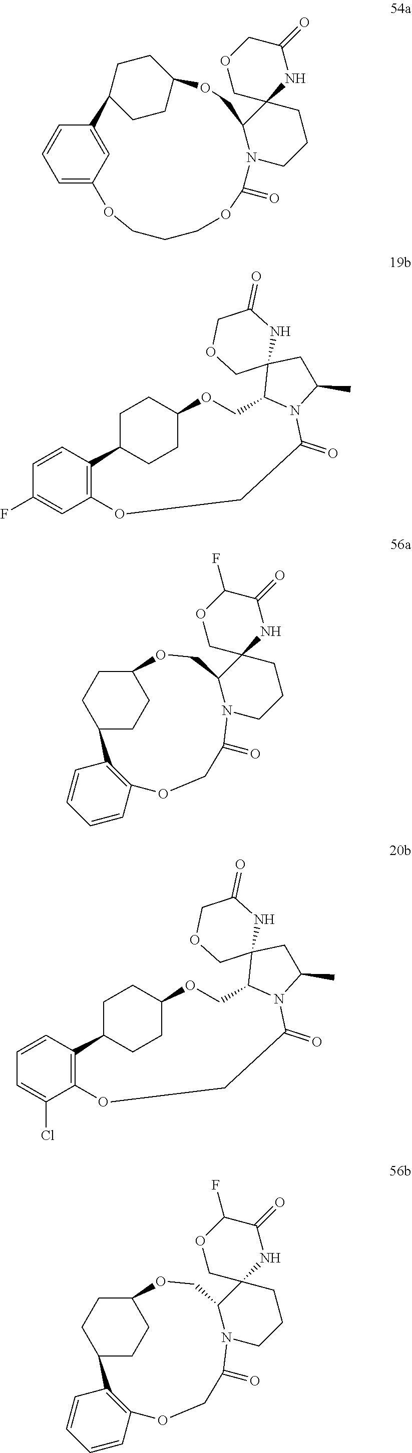

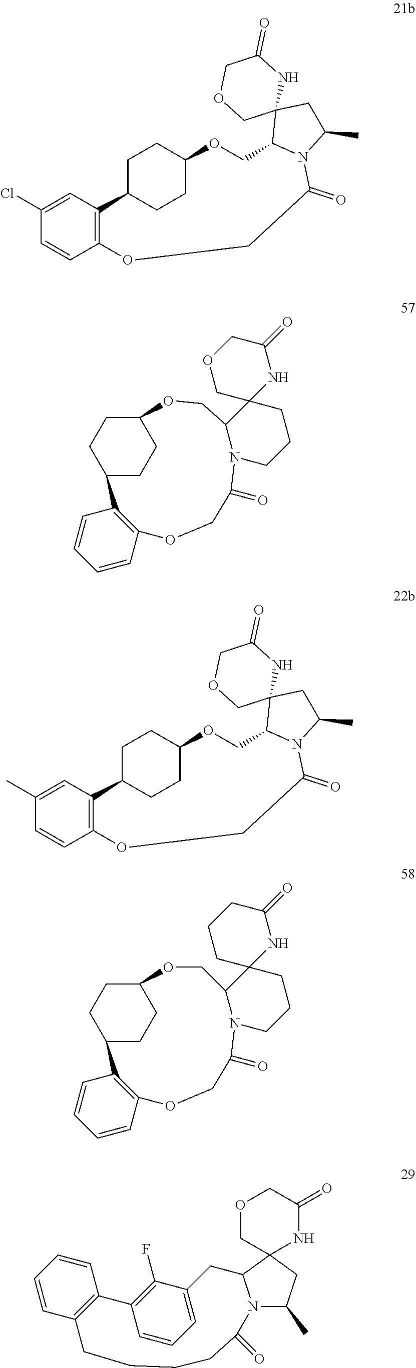

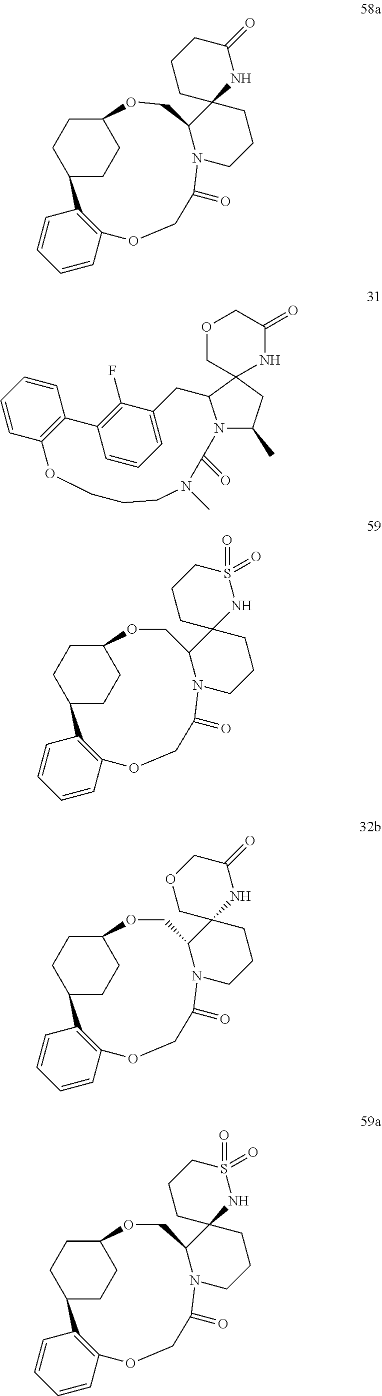

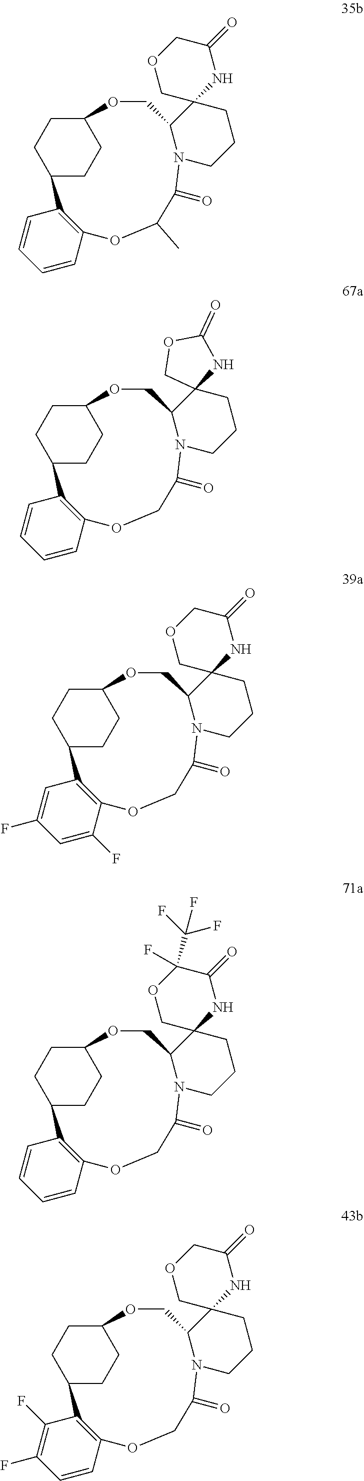 [SMILES: CC1Oc2ccccc2[C@H]2CC[C@H](CC2)OC[C@H]2N(CCC[C@]23COCC(=O)N3)C1=O ; O=C1N[C@]2(CCCN3C(=O)COc4ccccc4[C@H]4CC[C@H](CC4)OC[C@H]32)CO1 ; O=C1COC[C@@]2(CCCN3C(=O)COc4c(F)cc(F)cc4[C@H]4CC[C@H](CC4)OC[C@H]32)N1 ; O=C1COc2ccccc2[C@H]2CC[C@H](CC2)OC[C@@H]2N1CCC[C@@]21CO[C@](F)(C(F)(F)F)C(=O)N1 ; O=C1COC[C@]2(CCCN3C(=O)COc4ccc(F)c(F)c4[C@H]4CC[C@H](CC4)OC[C@@H]32)N1]

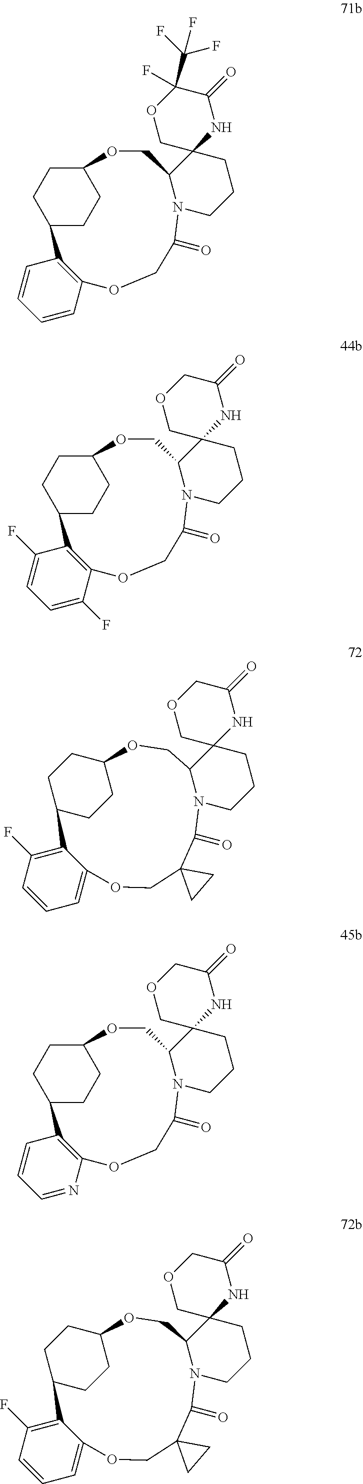 [SMILES: O=C1COc2ccccc2[C@H]2CC[C@H](CC2)OC[C@@H]2N1CCC[C@@]21CO[C@@](F)(C(F)(F)F)C(=O)N1 ; O=C1COC[C@]2(CCCN3C(=O)COc4c(F)ccc(F)c4[C@H]4CC[C@H](CC4)OC[C@@H]32)N1 ; O=C1COCC2(CCCN3C(=O)C4(CC4)COc4cccc(F)c4[C@H]4CC[C@H](CC4)OCC32)N1 ; O=C1COC[C@]2(CCCN3C(=O)COc4ncccc4[C@H]4CC[C@H](CC4)OC[C@@H]32)N1 ; O=C1COC[C@@]2(CCCN3C(=O)C4(CC4)COc4cccc(F)c4[C@H]4CC[C@H](CC4)OC[C@H]32)N1]

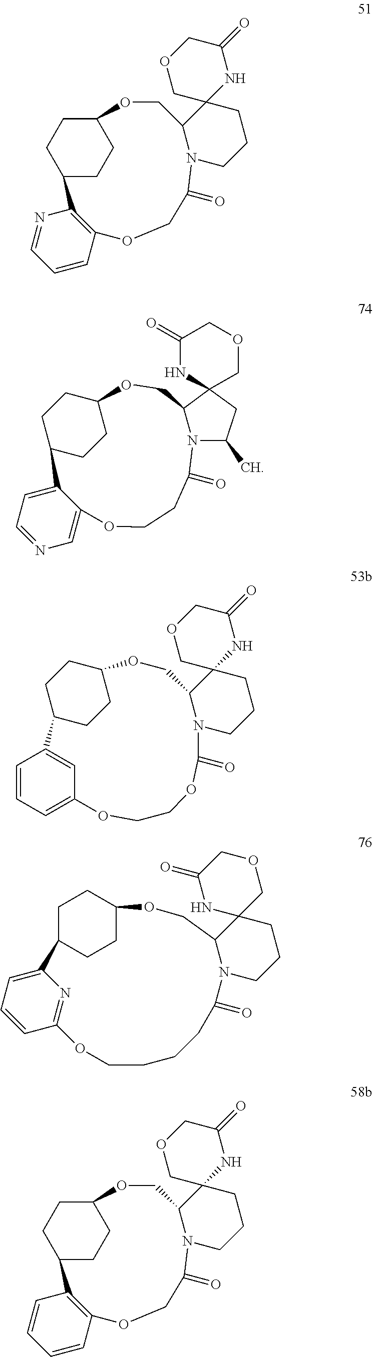

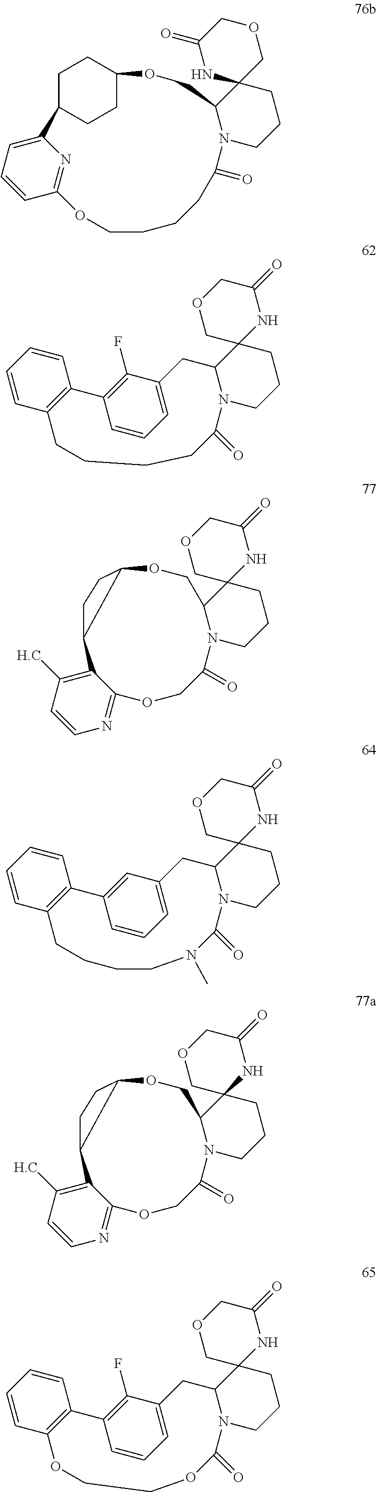

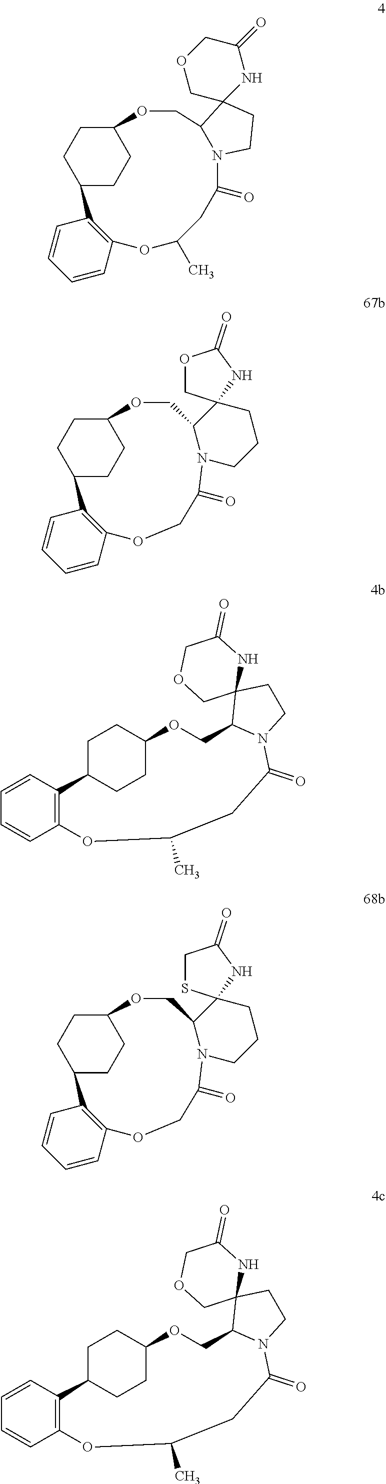 [SMILES: CC1CC(=O)N2CCC3(COCC(=O)N3)C2CO[C@H]2CC[C@H](CC2)c2ccccc2O1 ; O=C1N[C@@]2(CCCN3C(=O)COc4ccccc4[C@H]4CC[C@H](CC4)OC[C@@H]32)CO1 ; C[C@H]1CC(=O)N2CC[C@@]3(COCC(=O)N3)[C@@H]2CO[C@H]2CC[C@H](CC2)c2ccccc2O1 ; O=C1CS[C@]2(CCCN3C(=O)COc4ccccc4[C@H]4CC[C@H](CC4)OC[C@H]32)N1 ; C[C@@H]1CC(=O)N2CC[C@@]3(COCC(=O)N3)[C@@H]2CO[C@H]2CC[C@H](CC2)c2ccccc2O1]

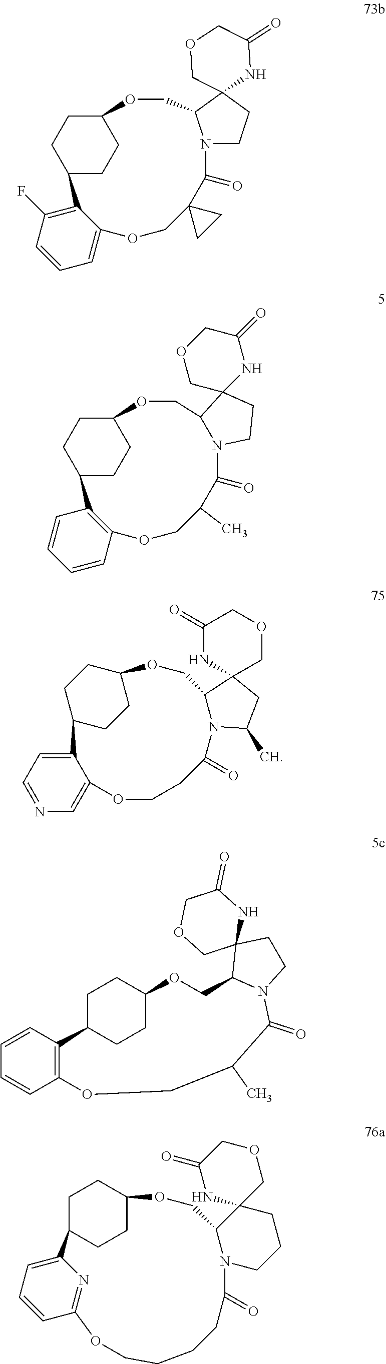

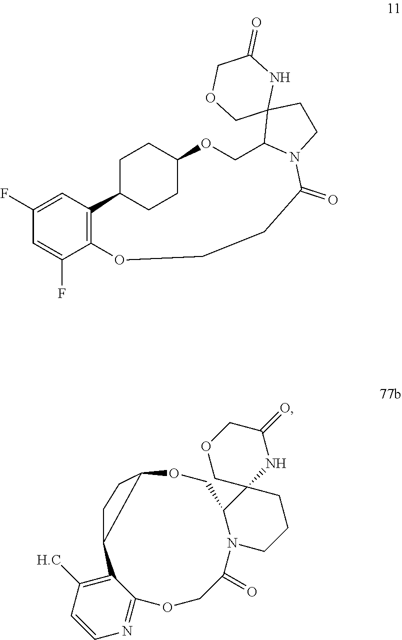

or a pharmaceutically acceptable salt thereof.

In some embodiments, the present disclosure provides a compound selected from the group consisting of:

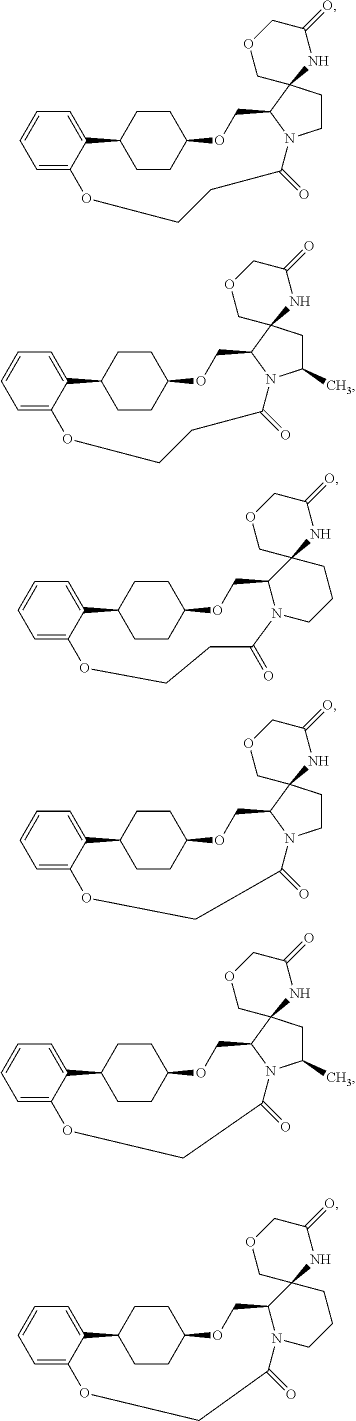

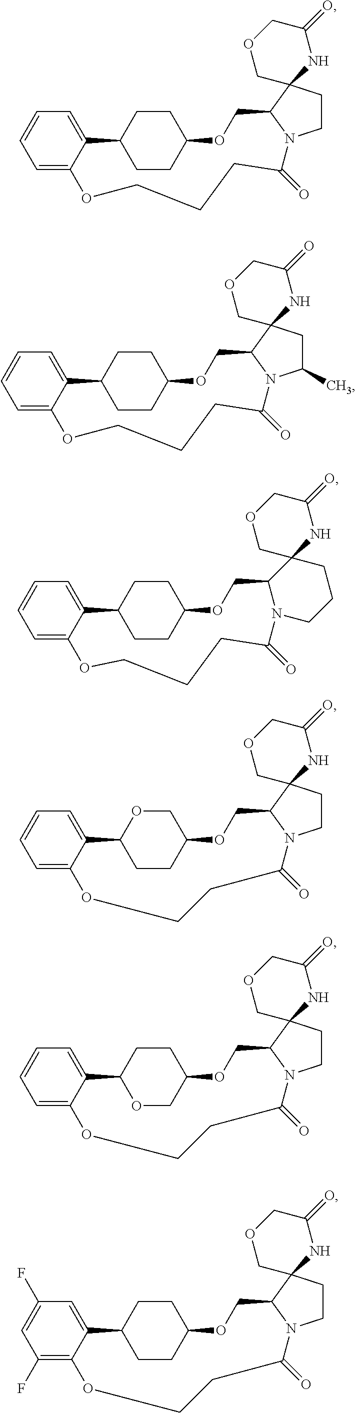 [SMILES: O=C1COC[C@@]2(CCN3C(=O)CCCOc4ccccc4[C@H]4CC[C@H](CC4)OC[C@H]32)N1 ; C[C@@H]1C[C@@]2(COCC(=O)N2)[C@@H]2CO[C@H]3CC[C@H](CC3)c3ccccc3OCCCC(=O)N12 ; O=C1COC[C@@]2(CCCN3C(=O)CCCOc4ccccc4[C@H]4CC[C@H](CC4)OC[C@H]32)N1 ; O=C1COC[C@@]2(CCN3C(=O)CCOc4ccccc4[C@@H]4CC[C@@H](CO4)OC[C@H]32)N1 ; O=C1COC[C@@]2(CCN3C(=O)CCOc4ccccc4[C@H]4CC[C@H](CO4)OC[C@H]32)N1 ; O=C1COC[C@@]2(CCN3C(=O)CCOc4c(F)cc(F)cc4[C@H]4CC[C@H](CC4)OC[C@H]32)N1]

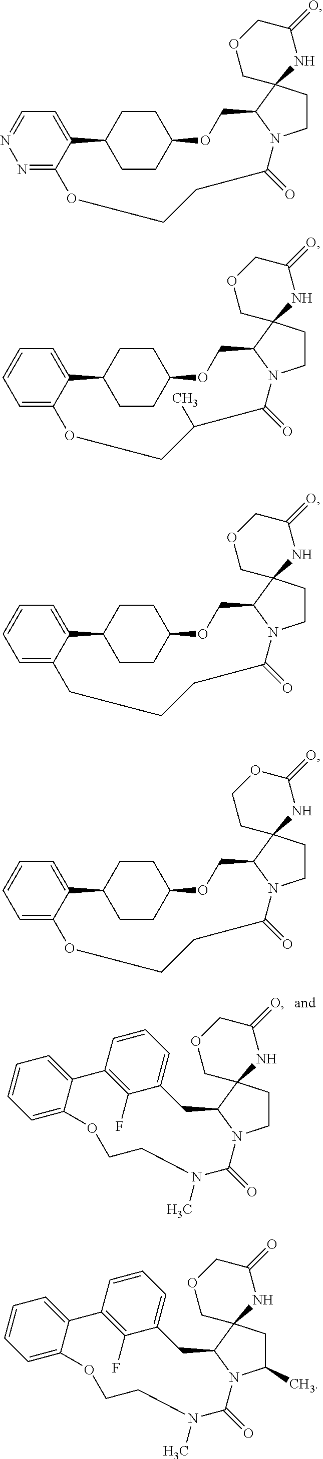

In some embodiments, the compounds disclosed herein are a racemic mixture. In some embodiments, the compounds disclosed herein are enriched in one enantiomer. In some embodiments, the compounds disclosed herein are enriched in one enantiomer and substantially free of the opposite enantiomer. In some embodiments, the compounds disclosed herein have an enantiomeric excess of about or greater than about 55%, about or greater than about 60%, about or greater than about 65%, about or greater than about 70%, about or greater than about 75%, about or greater than about 80%, about or greater than about 85%, about or greater than about 90%, about or greater than about 91%, about or greater than about 92%, about or greater than about 93%, about or greater than about 94%, about or greater than about 95%, about or greater than about 96%, about or greater than about 97%, about or greater than about 98%, about or greater than about 98.5%, about or greater than about 99%, about or greater than about 99.5%, or more, including all subranges and values therebetween. In some embodiments, the compounds of the present disclosure are provided as a mixture of diastereomers. In some embodiments, a diastereomer of a compound of the present disclosure is provided substantially free of other possible diastereomer(s). The present disclosure includes tautomers of any compounds described herein.

In some embodiments, provided herein is one or more compounds selected from Table 1 or a pharmaceutically acceptable salt thereof, or a stereoisomer thereof.

In some embodiments, provided herein is one or more compounds selected from Table 1 or a pharmaceutically acceptable salt thereof, or an enantiomer thereof.

In some embodiments, provided herein is one or more compounds selected from Table 1 or a pharmaceutically acceptable salt thereof, or a diastereomer, or mixture of diastereomers thereof.

In some embodiments, provided herein is one or more compounds selected from Table 1.

In some embodiments, provided herein is one or more pharmaceutically acceptable salts of a compound selected from Table 1.

TABLE 1
Compounds

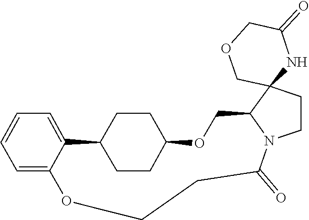 [SMILES: O=C1COC[C@@]2(CCN3C(=O)CCOc4ccccc4[C@H]4CC[C@H](CC4)OC[C@H]32)N1]

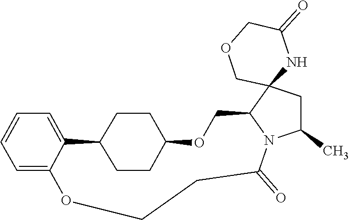 [SMILES: C[C@@H]1C[C@@]2(COCC(=O)N2)[C@@H]2CO[C@H]3CC[C@H](CC3)c3ccccc3OCCC(=O)N12]

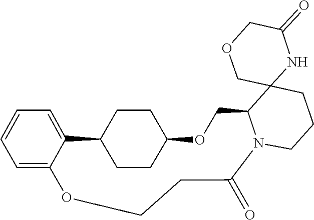 [SMILES: O=C1COCC2(CCCN3C(=O)CCOc4ccccc4[C@H]4CC[C@H](CC4)OC[C@H]32)N1]

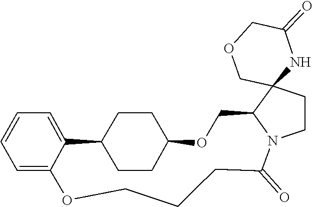 [SMILES: O=C1COC[C@@]2(CCN3C(=O)CCCOc4ccccc4[C@H]4CC[C@H](CC4)OC[C@H]32)N1]

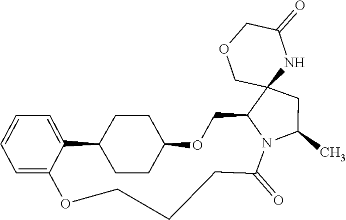 [SMILES: C[C@@H]1C[C@@]2(COCC(=O)N2)[C@@H]2CO[C@H]3CC[C@H](CC3)c3ccccc3OCCCC(=O)N12]

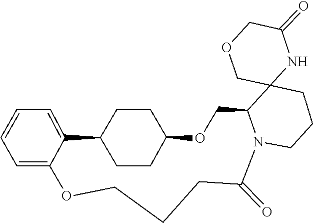 [SMILES: O=C1COCC2(CCCN3C(=O)CCCOc4ccccc4[C@H]4CC[C@H](CC4)OC[C@H]32)N1]

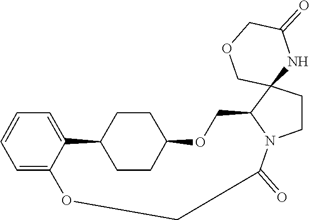 [SMILES: O=C1COC[C@@]2(CCN3C(=O)COc4ccccc4[C@H]4CC[C@H](CC4)OC[C@H]32)N1]

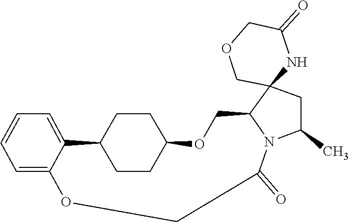 [SMILES: C[C@@H]1C[C@@]2(COCC(=O)N2)[C@@H]2CO[C@H]3CC[C@H](CC3)c3ccccc3OCC(=O)N12]

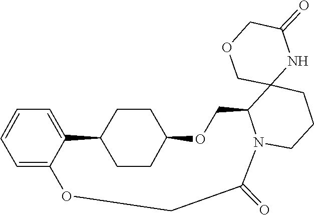 [SMILES: O=C1COCC2(CCCN3C(=O)COc4ccccc4[C@H]4CC[C@H](CC4)OC[C@H]32)N1]

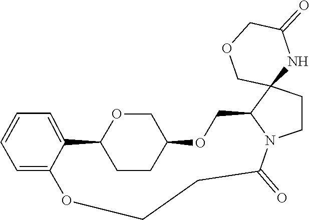 [SMILES: O=C1COC[C@@]2(CCN3C(=O)CCOc4ccccc4[C@@H]4CC[C@@H](CO4)OC[C@H]32)N1]

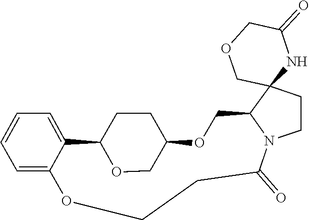 [SMILES: O=C1COC[C@@]2(CCN3C(=O)CCOc4ccccc4[C@H]4CC[C@H](CO4)OC[C@H]32)N1]

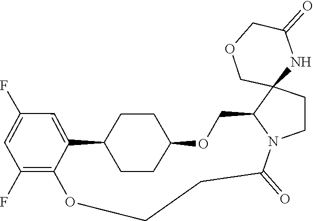 [SMILES: O=C1COC[C@@]2(CCN3C(=O)CCOc4c(F)cc(F)cc4[C@H]4CC[C@H](CC4)OC[C@H]32)N1]

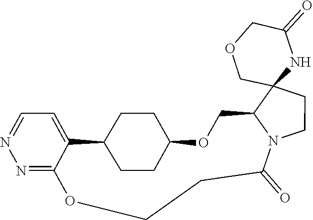 [SMILES: O=C1COC[C@@]2(CCN3C(=O)CCOc4nnccc4[C@H]4CC[C@H](CC4)OC[C@H]32)N1]

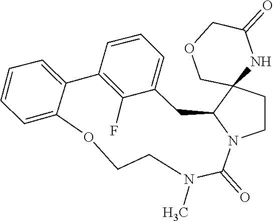

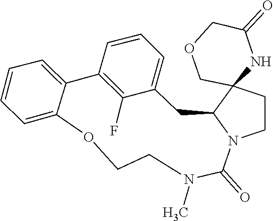

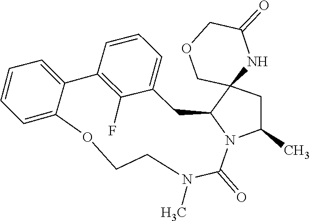 [SMILES: C[C@@H]1C[C@@]2(COCC(=O)N2)[C@@H]2Cc3cccc(c3F)-c3ccccc3OCCN(C)C(=O)N12]

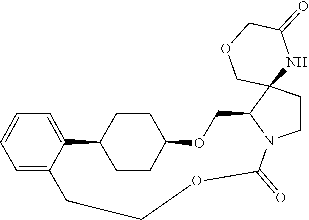 [SMILES: O=C1COC[C@@]2(CCN3C(=O)OCCc4ccccc4[C@H]4CC[C@H](CC4)OC[C@H]32)N1]

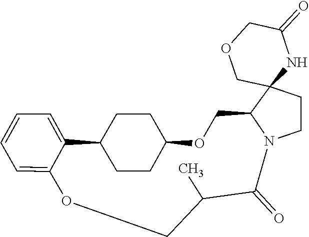 [SMILES: CC1COc2ccccc2[C@H]2CC[C@H](CC2)OC[C@@H]2N(CC[C@@]23COCC(=O)N3)C1=O]

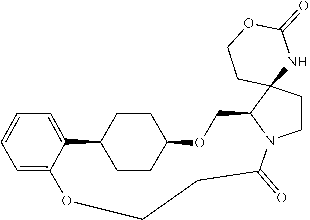 [SMILES: O=C1N[C@@]2(CCO1)CCN1C(=O)CCOc3ccccc3[C@H]3CC[C@H](CC3)OC[C@H]12]

In some embodiments, the present disclosure provides a compound provided in Table 2 or a pharmaceutically acceptable salt thereof. In some embodiments, the present disclosure provides a compound provided in Table 2 or a pharmaceutically acceptable salt thereof having A or B activity. In some embodiments, the present disclosure provides a compound provided in Table 2 or a pharmaceutically acceptable salt thereof having A activity.

### Compositions

The present disclosure provides pharmaceutical compositions for modulating orexin receptor (e.g., orexin type 2 receptor) in a subject. In some embodiments, a pharmaceutical composition comprises one or more compounds of the present disclosure (e.g., a compound of Formula (I), Formula (I-1), Formula (I-2), Formula (IA), Formula (IA-1), Formula (IA-1-1), Formula (IA-2), Formula (IA-2-1), Formula (IA-3), Formula (IA-4), Formula (IA-5), Formula (IB), Formula (IB-1), Formula (IB-2), Formula (IC), Formula (IC-1), Formula (IC-2), Formula (IC-3), Formula (IC-4), Formula (IC-5), Formula (IC-6), Formula (ID), Formula (ID-1), Formula (ID-2), Formula (IE), Formula, (IF), Table 1, or Table 2) or a pharmaceutically acceptable salt thereof.

In some embodiments of the present disclosure, a pharmaceutical composition comprises a therapeutically effective amounts of one or more compounds of the present disclosure (e.g., a compound of Formula (I), Formula (I-1), Formula (I-2), Formula (IA), Formula (IA-1), Formula (IA-2), Formula (IB), Formula (IB-1), Formula (IB-2), Formula (IC), Formula (IC-1), Formula (IC-2), Formula (IC-3), Formula (ID), Formula (ID-1), Formula (ID-2), Formula (IE), Formula, (IF), Table 1, or Table 2) or a pharmaceutically acceptable salt thereof.

In some embodiments, a pharmaceutical composition, as described herein, comprises one or more compounds selected from Table 1, or a pharmaceutically acceptable salt thereof or stereoisomer thereof.

In some embodiments, a pharmaceutical composition, as described herein, comprises one or more compounds selected from Table 2, or a pharmaceutically acceptable salt thereof.

In some embodiments, the present disclosure provides a pharmaceutical composition comprising a compound disclosed herein (e.g., a compound of Formula (I), Formula (I-1), Formula (I-2), Formula (IA), Formula (IA-1), Formula (IA-1-1), Formula (IA-2), Formula (IA-2-1), Formula (IA-3), Formula (IA-4), Formula (IA-5), Formula (IB), Formula (IB-1), Formula (IB-2), Formula (IC), Formula (IC-1), Formula (IC-2), Formula (IC-3), Formula (IC-4), Formula (IC-5), Formula (IC-6), Formula (ID), Formula (ID-1), Formula (ID-2), Formula (IE), Formula, (IF), Table 1, or Table 2), or a pharmaceutically acceptable salt thereof; and one or more pharmaceutically acceptable excipients.

In some embodiments of the present disclosure, a pharmaceutical composition comprising one or more compounds of the present disclosure (e.g., a compound of Formula (I), Formula (I-1), Formula (I-2), Formula (IA), Formula (IA-1), Formula (IA-1-1), Formula (IA-2), Formula (IA-2-1), Formula (IA-3), Formula (IA-4), Formula (IA-5), Formula (IB), Formula (IB-1), Formula (IB-2), Formula (IC), Formula (IC-1), Formula (IC-2), Formula (IC-3), Formula (IC-4), Formula (IC-5), Formula (IC-6), Formula (ID), Formula (ID-1), Formula (ID-2), Formula (IE), Formula, (IF), Table 1, or Table 2) or a pharmaceutically acceptable salt thereof, and a pharmaceutically acceptable excipient or adjuvant is provided. The pharmaceutically acceptable excipients and adjuvants are added to the composition or formulation for a variety of purposes. In some embodiments, a pharmaceutical composition comprising one or more compounds disclosed herein, or a pharmaceutically acceptable salt thereof, further comprise a pharmaceutically acceptable carrier. In some embodiments, a pharmaceutically acceptable carrier includes a pharmaceutically acceptable excipient, binder, and/or diluent. In some embodiments, suitable pharmaceutically acceptable carriers include, but are not limited to, inert solid fillers or diluents and sterile aqueous or organic solutions. In some embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like.

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

Generally, the compounds of the present disclosure are administered in a therapeutically effective amount. The amount of the compound actually administered will typically be determined by a physician, in the light of the relevant circumstances, including the condition to be treated, the chosen route of administration, the actual compound-administered, the age, weight, and response of the individual patient, the severity of the patient's symptoms, and the like.

### Methods of Treatment

The compounds of the present disclosure find use in any number of methods. For example, in some embodiments the compounds are useful in methods for modulating an orexin receptor, e.g., orexin type 2 receptor. Accordingly, in some embodiments, the present disclosure provides the use of any one of the foregoing compounds of Formula (I), Formula (I-1), Formula (I-2), Formula (IA), Formula (IA-1), Formula (IA-1-1), Formula (IA-2), Formula (IA-2-1), Formula (IA-3), Formula (IA-4), Formula (IA-5), Formula (IB), Formula (IB-1), Formula (IB-2), Formula (IC), Formula (IC-1), Formula (IC-2), Formula (IC-3), Formula (IC-4), Formula (IC-5), Formula (IC-6), Formula (ID), Formula (ID-1), Formula (ID-2), Formula (IE), Formula, (IF), Table 1, or Table 2 or a pharmaceutically acceptable salt thereof, for modulating orexin receptor (e.g., orexin type 2 receptor) activity. For example in some embodiments, modulating orexin receptor (e.g., orexin type 2 receptor) activity is in a mammalian cell. Modulating orexin receptor (e.g., orexin type 2 receptor) activity can be in a subject in need thereof (e.g., a mammalian subject, such as a human) and for treatment of any of the described conditions or diseases.

In some embodiments, the modulating orexin receptor (e.g., orexin type 2 receptor) activity is binding. In some embodiments, the modulating orexin receptor (e.g., orexin type 2 receptor) activity is agonizing or stimulating the orexin receptor.

In some embodiments, the present disclosure provides methods of treating a disease or disorder that is treatable by administration of an Orexin agonist, the method comprising administering a therapeutically effective amount of one or more compounds of the present disclosure (e.g., compounds of Formula (I), Formula (I-1), Formula (I-2), Formula (IA), Formula (IA-1), Formula (IA-1-1), Formula (IA-2), Formula (IA-2-1), Formula (IA-3), Formula (IA-4), Formula (IA-5), Formula (IB), Formula (IB-1), Formula (IB-2), Formula (IC), Formula (IC-1), Formula (IC-2), Formula (IC-3), Formula (IC-4), Formula (IC-5), Formula (IC-6), Formula (ID), Formula (ID-1), Formula (ID-2), Formula (IE), Formula, (IF), Table 1, or Table 2) to a subject in need thereof.

In some embodiments, the present disclosure provides methods of treating a disease or disorder that is treatable by administration of an Orexin agonist, the method comprising administering a composition comprising a therapeutically effective amount of one or more compounds of the present disclosure (e.g., compounds of Formula (I), Formula (I-1), Formula (I-2), Formula (IA), Formula (IA-1), Formula (IA-1-1), Formula (IA-2), Formula (IA-2-1), Formula (IA-3), Formula (IA-4), Formula (IA-5), Formula (IB), Formula (IB-1), Formula (IB-2), Formula (IC), Formula (IC-1), Formula (IC-2), Formula (IC-3), Formula (IC-4), Formula (IC-5), Formula (IC-6), Formula (ID), Formula (ID-1), Formula (ID-2), Formula (IE), Formula, (IF), Table 1, or Table 2) to a subject in need thereof.

In some embodiments, the compounds of the present disclosure are used for treating, preventing, ameliorating, controlling or reducing the risk of a variety of disorders associated with orexin receptors, including one or more of the following conditions or diseases: narcolepsy, narcolepsy syndrome accompanied by narcolepsy-like symptoms, cataplexy in narcolepsy, excessive daytime sleepiness (EDS) in narcolepsy, hypersomnia, idiopathic hypersomnia, repeatability hypersomnia, intrinsic hypersomnia, hypersomnia accompanied by daytime hypersomnia, interrupted sleep, sleep apnea, hypersomnia associated with sleep apnea, nocturnal myoclonus, disturbances of consciousness, such as coma, REM sleep interruptions, jet-lag, excessive daytime sleepiness, shift workers' sleep disturbances, dyssomnias, sleep disorders, sleep disturbances, hypersomnia associated with depression, emotional/mood disorders, drug use, Alzheimer's disease or cognitive impairment, Parkinson's disease, Guillain-Barre syndrome, Kleine Levin syndrome, and sleep disorders which accompany aging, muscular dystrophies, immune-mediated diseases; Alzheimer's sundowning; conditions associated with circadian rhythmicity as well as mental and physical disorders associated with travel across time zones and with rotating shift-work schedules; fibromyalgia; cardiac failure; diseases related to bone loss; sepsis; syndromes which are manifested by non-restorative sleep and muscle pain or sleep apnea which is associated with respiratory disturbances during sleep; conditions which result from a diminished quality of sleep; and other diseases related to general orexin system dysfunction. In some embodiments, compounds of the present disclosure are useful for treating, preventing, ameliorating, controlling or reducing the risk of a variety of narcolepsy, idiopathic hypersomnia, hypersomnia, sleep apnea syndrome, narcolepsy syndrome accompanied by narcolepsy-like symptoms, hypersomnia syndrome accompanied by daytime hypersomnia (e.g., Parkinson's disease, Guillain-Barre syndrome and Kleine Levin syndrome), Alzheimer's disease obesity, insulin resistance syndrome, cardiac failure, diseases related to bone loss, sepsis, disturbance of consciousness such as coma and the like, side effects and complications due to anesthesia, and the like, or anesthetic antagonist.

In some embodiments, a compound of the present disclosure (e.g., a compound of Formula (I), Formula (I-1), Formula (I-2), Formula (IA), Formula (IA-1), Formula (IA-1-1), Formula (IA-2), Formula (IA-2-1), Formula (IA-3), Formula (IA-4), Formula (IA-5), Formula (IB), Formula (IB-1), Formula (IB-2), Formula (IC), Formula (IC-1), Formula (IC-2), Formula (IC-3), Formula (IC-4), Formula (IC-5), Formula (IC-6), Formula (ID), Formula (ID-1), Formula (ID-2), Formula (IE), Formula, (IF), Table 1, or Table 2), a pharmaceutically acceptable salt thereof, or a composition thereof is used to treat diseases or disorders or symptoms associated with excessive sleepiness in a subject in need thereof. In some embodiments, the excessive sleepiness is caused by any one of the following: insufficient quality or quantity of night time sleep; misalignments of the body's circadian pacemaker with the environment (e.g., caused by requirement to remain awake at night for employment such as shift work or personal obligations such as caretaker for sick, young or old family members), such as jet lag, shift work and other circadian rhythm sleep disorders; another underlying sleep disorder, such as narcolepsy (e.g., narcolepsy type 1, narcolepsy type 2, probable narcolepsy), sleep apnea (e.g., obstructive sleep apnea, obstructive sleep apnea with use of continuous positive airway pressure), idiopathic hypersomnia, idiopathic excessive sleepiness, and restless legs syndrome; disorders, such as clinical depression or atypical depression; tumors; head trauma; anemia; kidney failure; hypothyroidism; injury to the central nervous system; drug abuse; genetic vitamin deficiency, such as biotin deficiency; and particular classes of prescription and over the counter medication.

In some embodiments, a compound of the present disclosure (e.g., a compound of Formula (I), Formula (I-1), Formula (I-2), Formula (IA), Formula (IA-1), Formula (IA-1-1), Formula (IA-2), Formula (IA-2-1), Formula (IA-3), Formula (IA-4), Formula (IA-5), Formula (IB), Formula (IB-1), Formula (IB-2), Formula (IC), Formula (IC-1), Formula (IC-2), Formula (IC-3), Formula (IC-4), Formula (IC-5), Formula (IC-6), Formula (ID), Formula (ID-1), Formula (ID-2), Formula (IE), Formula, (IF), Table 1, or Table 2), a pharmaceutically acceptable salt thereof, or a composition thereof is used to treat any one of the following: shift work disorder; shift work sleep disorder; and jet lag syndrome. In some embodiments, the methods and uses herein are used to treat any one of the following: narcolepsy type 1, narcolepsy type 2, probable narcolepsy, idiopathic hypersomnia, idiopathic excessive sleepiness, hypersomnia, hypersomnolence, sleep apnea syndrome (e.g., obstructive sleep apnea, obstructive sleep apnea with use of continuous positive airway pressure); or disturbance of consciousness such as coma and the like; and narcolepsy syndrome accompanied by narcolepsy-like symptoms; hypersomnolence or hypersomnia syndrome accompanied by daytime hypersomnia (e.g., Parkinson's disease, Guillain-Barre syndrome and Kleine Levin syndrome); excessive daytime sleepiness in Parkinson's disease, Prader-Willi Syndrome, depressions (depression, atypical depression, major depressive disorder, treatment resistant depression), ADHD, sleep apnea syndrome (e.g., obstructive sleep apnea, obstructive sleep apnea with use of continuous positive airway pressure) and other disorders of vigilance; residual excessive daytime sleepiness in sleep apnea syndrome (e.g., obstructive sleep apnea, obstructive sleep apnea with use of continuous positive airway pressure); and the like. Narcolepsy (e.g., narcolepsy type 1, narcolepsy type 2, probable narcolepsy) may be diagnosed by diagnostic criteria generally used in the field, e.g., The third edition of the International Classification of Sleep Disorders (ICSD-3) and the fifth edition of the Diagnostic and Statistical Manual of Mental Disorders (DSM-5). In some embodiments, the excessive sleepiness is excessive daytime sleepiness or excessive sleepiness during working hours, or excessive sleepiness or reduced quantity of sleep which is caused by requirement to remain awake at night for employment (e.g., shift work) or personal obligations (e.g., caretaker for sick, young or old family members). In some embodiments, the subject suffers from the diseases or disorders or symptoms associated with excessive sleepiness. In some embodiments, the subject is sleep-deprived subject, subject with excessive sleepiness, subject with disruptive regular sleep cycle, or subject with a need to decrease sleepiness. In some embodiments, the present disclosure provides methods for decreasing or treating excessive sleepiness. In some embodiments, the excessive sleepiness is caused by narcolepsy type 1, narcolepsy type 2 or idiopathic hypersomnia. In some embodiments, the excessive sleepiness is caused by obstructive sleep apnea despite the use of continuous positive airway pressure (CPAP). In some embodiments, methods for increasing wakefulness in a subject in need thereof is provided. In some embodiments, the orexin level in the subject is not compromised or partially compromised.

In some embodiments of the present disclosure, a method for the treatment of a sleep disorder (e.g., as disclosed herein) in a subject in need thereof is provided, comprising administering a compound of the present disclosure (e.g., a compound Formula (I), Formula (I-1), Formula (I-2), Formula (IA), Formula (IA-1), Formula (IA-1-1), Formula (IA-2), Formula (IA-2-1), Formula (IA-3), Formula (IA-4), Formula (IA-5), Formula (IB), Formula (IB-1), Formula (IB-2), Formula (IC), Formula (IC-1), Formula (IC-2), Formula (IC-3), Formula (IC-4), Formula (IC-5), Formula (IC-6), Formula (ID), Formula (ID-1), Formula (ID-2), Formula (IE), Formula, (IF), Table 1, or Table 2), a pharmaceutically acceptable salt thereof, or a composition thereof, to the subject. In some embodiments, a compound of the present disclosure (e.g., a compound of Formula (I), Formula (I-1), Formula (I-2), Formula (IA), Formula (IA-1), Formula (IA-1-1), Formula (IA-2), Formula (IA-2-1), Formula (IA-3), Formula (IA-4), Formula (IA-5), Formula (IB), Formula (IB-1), Formula (IB-2), Formula (IC), Formula (IC-1), Formula (IC-2), Formula (IC-3), Formula (IC-4), Formula (IC-5), Formula (IC-6), Formula (ID), Formula (ID-1), Formula (ID-2), Formula (IE), Formula, (IF), Table 1, or Table 2), or a pharmaceutically acceptable salt thereof, is used to treat a subject with a sleep disorder, to treat a sleep disorder, or to treat the symptoms of a sleep disorder.

In some embodiments of the present disclosure, a method for the treatment of narcolepsy in a subject in need thereof is provided, comprising administering a compound of the present disclosure (e.g., a compound of Formula (I), Formula (I-1), Formula (I-2), Formula (IA), Formula (IA-1), Formula (IA-1-1), Formula (IA-2), Formula (IA-2-1), Formula (IA-3), Formula (IA-4), Formula (IA-5), Formula (IB), Formula (IB-1), Formula (IB-2), Formula (IC), Formula (IC-1), Formula (IC-2), Formula (IC-3), Formula (IC-4), Formula (IC-5), Formula (IC-6), Formula (ID), Formula (ID-1), Formula (ID-2), Formula (IE), Formula, (IF), Table 1, or Table 2), or a pharmaceutically acceptable salt thereof, to a subject in need thereof. In some embodiments, a compound of the present disclosure (e.g., a compound of Formula (I), Formula (I-1), Formula (I-2), Formula (IA), Formula (IA-1), Formula (IA-1-1), Formula (IA-2), Formula (IA-2-1), Formula (IA-3), Formula (IA-4), Formula (IA-5), Formula (IB), Formula (IB-1), Formula (IB-2), Formula (IC), Formula (IC-1), Formula (IC-2), Formula (IC-3), Formula (IC-4), Formula (IC-5), Formula (IC-6), Formula (ID), Formula (ID-1), Formula (ID-2), Formula (IE), Formula, (IF), Table 1, or Table 2), or a pharmaceutically acceptable salt thereof, is used to treat a subject with narcolepsy, to treat narcolepsy, or to treat the symptoms of narcolepsy.

In some embodiments of the present disclosure, a method for the treatment of idiopathic hypersomnia (IH) in a subject in need thereof is provided, comprising administering a compound of the present disclosure (e.g., a compound Formula (I), Formula (I-1), Formula (I-2), Formula (IA), Formula (IA-1), Formula (IA-1-1), Formula (IA-2), Formula (IA-2-1), Formula (IA-3), Formula (IA-4), Formula (IA-5), Formula (IB), Formula (IB-1), Formula (IB-2), Formula (IC), Formula (IC-1), Formula (IC-2), Formula (IC-3), Formula (IC-4), Formula (IC-5), Formula (IC-6), Formula (ID), Formula (ID-1), Formula (ID-2), Formula (IE), Formula, (IF), Table 1, or Table 2), or a pharmaceutically acceptable salt thereof, to a subject in need thereof. In some embodiments, a compound of the present disclosure (e.g., a compound Formula (I), Formula (I-1), Formula (I-2), Formula (IA), Formula (IA-1), Formula (IA-1-1), Formula (IA-2), Formula (IA-2-1), Formula (IA-3), Formula (IA-4), Formula (IA-5), Formula (IB), Formula (IB-1), Formula (IB-2), Formula (IC), Formula (IC-1), Formula (IC-2), Formula (IC-3), Formula (IC-4), Formula (IC-5), Formula (IC-6), Formula (ID), Formula (ID-1), Formula (ID-2), Formula (IE), Formula, (IF), Table 1, or Table 2), or a pharmaceutically acceptable salt thereof, is used to treat a subject with IH, to treat IH, or to treat the symptoms of IH.

### EXAMPLES

The disclosure now being generally described, it will be more readily understood by reference to the following examples which are included merely for purposes of illustration of certain aspects and embodiments of the present disclosure and are not intended to limit the disclosure.

The compounds of the present disclosure can be synthesized using the methods as hereinafter described below, together with synthetic methods known in the art of synthetic organic chemistry or variations thereon as appreciated by those skilled in the art.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene and Wuts, Protective Groups in Organic Synthesis, 44th. Ed., Wiley and Sons, 2006, as well as in Jerry March, Advanced Organic Chemistry, 4^{th} edition, John Wiley and Sons, publisher, New York, 1992 which are incorporated herein by reference in their entirety.

### Abbreviations

AcOH acetic acid
DCM dichloromethane
DIPEA N,N-diisopropylethylamine
DMPU N,N′-dimethylpropyleneurea
DMSO dimethyl sulfoxide
EtOAc ethyl acetate
IPA isopropyl alcohol
LDA lithium diisopropylamide
NMO N-methylmorpholine-N-Oxide
TEA triethylamine
TFA trifluoroacetic acid
TFAA trifluoroacetic anhydride
THF tetrahydrofuran

### General Synthesis

Compounds of the present disclosure can be synthesized using the following methods. General reaction conditions are given, and reaction products can be purified by generally known methods including silica gel chromatography using various organic solvents such as hexane, dichloromethane, ethyl acetate, methanol and the like or preparative reverse phase high pressure liquid chromatography.

### Analytical Conditions:

### Method A:

Column: Waters UPLC® BEH™ C18, Part No. 186002352, 2.1×100 mm, 1.7 μm
Column Temperature: 40° C.
Mobile Phase A: 2 mM ammonia bicarbonate, buffered to pH 10
Mobile Phase B: Acetonitrile
Injection volume: 1 μL
Gradient program: Flow rate 0.6 mL/minutes

| Time | A % | B % |
|---|---|---|
| 0.00 | 95.00 | 5.00 |
| 5.30 | 0 | 100 |
| 5.80 | 0 | 100 |
| 5.82 | 95.00 | 5.00 |
| 7.00 | 95.00 | 5.00 |

UV 215 nm, PDA spectrum 200-400 nm, step: 1 nm
MSD Scan Positive: 100-1000; Scan Positive Negative: 150-850; Scan Neg: 100-1000

### Method B:

Column: Phenomenex, Kinetex-XB C18, Part No. 00D-4498-AN, 2.1 mm×100 mm, 1.7 μm
Column temperature: 40° C.
Mobile Phase A: 0.1% Formic acid in water
Mobile Phase B: 0.1% Formic acid in acetonitrile
Injection volume: 1 μL
Gradient program: Flow rate 0.6 mL/minutes

| Time | A % | B % |
|---|---|---|
| 0.00 | 95 | 5 |
| 5.30 | 0 | 100 |
| 5.80 | 0 | 100 |
| 5.82 | 95 | 5 |
| 7.00 | 95 | 5 |

UV 215 nm, PDA spectrum 200-400 nm, step: 1 nm
MSD Scan Positive: 100-1000; Scan Positive Negative: 150-850

### Method C:

Column: Acquity UPLC CSH C18 (5.0 mm×2.1 mm I.d. 1.7 um) column
Column Temperature: 40° C.
Mobile Phase A: 0.1% Formic acid in water
Mobile Phase B: 0.1% Formic acid in acetonitrile
Gradient program: Flow rate 1 mL/minute

| Time | A % | B % |
|---|---|---|
| 0.00 | 97.00 | 3.00 |
| 1.50 | 0.10 | 99.90 |
| 1.90 | 0.10 | 99.90 |
| 2.00 | 97.0 | 3.00 |

### Method D:

Column: Kinetex EVO C18 (1.7 μm, 2.1×50 mm) column
Column Temperature: 40° C.
Mobile Phase A: 10 mM ammonia bicarbonate aq. solution adjusted to pH 10 with NH₃
Mobile Phase B: Acetonitrile
Gradient program: Flow rate 1 mL/minute

| Time | A % | B % |
|---|---|---|
| 0.00 | 97.00 | 3.00 |
| 1.50 | 0.10 | 99.90 |
| 1.90 | 0.10 | 99.90 |
| 2.00 | 97.0 | 3.00 |

### Synthesis of Spirocyclic Core

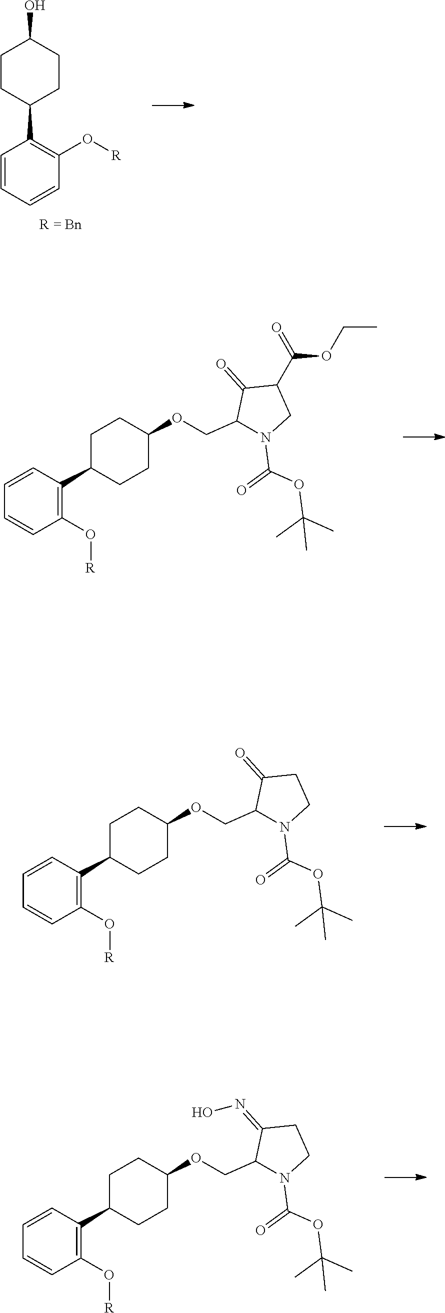

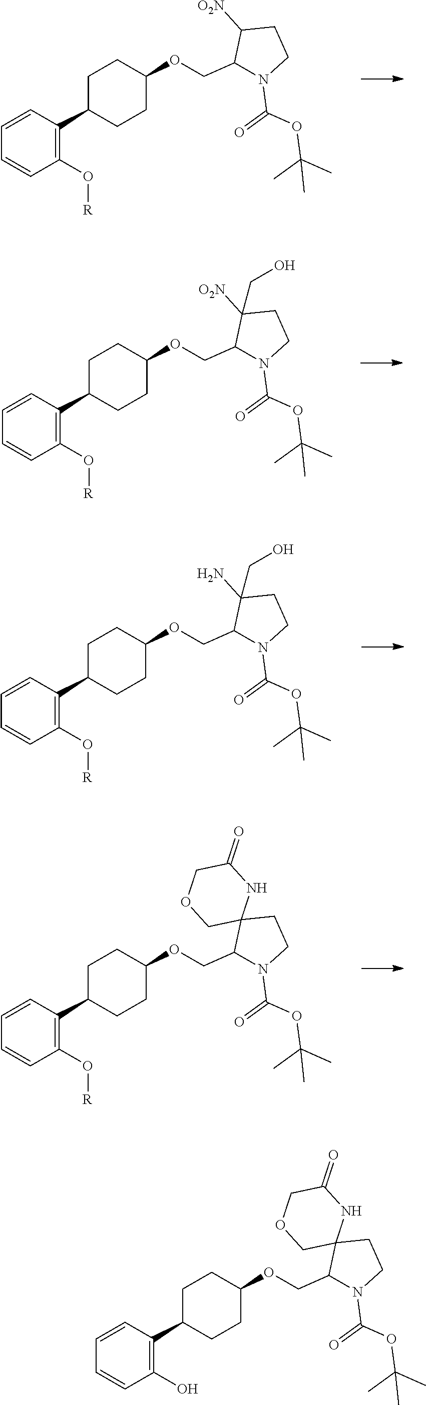

### Intermediate 1

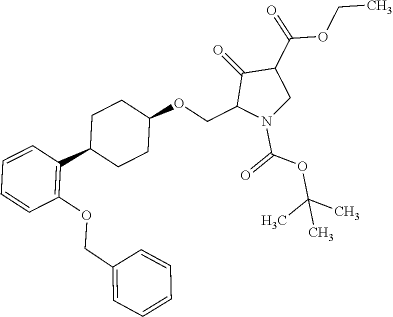 [SMILES: CCOC(=O)C1CN(C(=O)OC(C)(C)C)C(CO[C@H]2CC[C@@H](c3ccccc3OCc3ccccc3)CC2)C1=O]

### 1-tert-butyl 3-ethyl 4-oxo-5-({[(1s,4s)-4-[2-(benzyloxy)phenyl]cyclohexyl]oxy}methyl)pyrrolidine-1,3-dicarboxylate

In a flask, 2.4 M butyllithium (49 mL, 0.117 mol) was added to a stirred solution of N-(propan-2-yl) propan-2-amine (16 mL, 0.116 mol) in anhydrous THF (58 mL) at −78° C. The reaction was held at this temperature for 40 min. This freshly made LDA was added, via an addition funnel over 0.5 h, to a stirred solution of 1-tert-butyl 3-ethyl 4-oxopyrrolidine-1,3-dicarboxylate (13.70 g, 53.2 mmol) and 1,3-dimethylhexahydropyrimidin-2-one (27.20 g, 0.212 mol) in anhydrous THF (154 mL) at −78° C., the reaction temperature did not raise above −65° C. The solution was held at this temperature for 20 mins. A solution of 1-benzyloxy-2-[4-(chloromethoxy)cyclohexyl]benzene (17.60 g, 53.2 mmol) in anhydrous THF (37 mL) was added to the reaction mixture over 20 mins. The reaction mixture was stirred at −78° C. for 1 h, warmed up to room temperature, and stirred for 2 hours. The reaction was quenched with NH₄Cl, diluted with water (100 mL) and extracted with EtOAc (3×300 mL). The combined organic layers were dried over MgSO₄, filter, and concentrated in vacuo to afford the crude material. The crude material was purified by silica gel column chromatography (0-25% EtOAc in heptane), to afford the title compound (17.50 g) as a yellow oil. (M+Na)⁺ m/z: 574.3.

### Intermediate 2

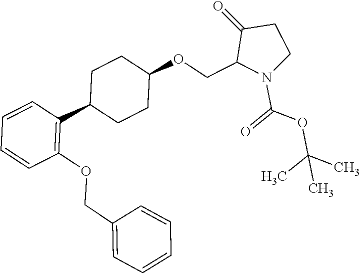 [SMILES: CC(C)(C)OC(=O)N1CCC(=O)C1CO[C@H]1CC[C@@H](c2ccccc2OCc2ccccc2)CC1]

### tert-butyl 3-oxo-2-({[(1s,4s)-4-[2-(benzyloxy)phenyl]cyclohexyl]oxy}methyl)pyrrolidine-1-carboxylate

Intermediate 1 (17.70 g, 32.1 mmol) in DMSO (148 mL) was added sodium chloride (3.50 g, 59.9 mmol) and water (15 mL) and reaction mixture was heated to 125° C. for 2.5 h. The reaction mixture was cooled to room temperature, quenched water (50 mL) and extracted with EtOAc (2×100 mL). The combined organic layers were washed with water (2×50 mL), brine (50 mL), dried over sodium sulfate, filtered, and evaporated to dryness to afford crude material. The crude material was purified by column chromatography (0-40% EtOAc in heptane), to afford the title compound (10.00 g) as a yellow oil. [M+Na]⁺ m/z 502.3.

### Intermediate 3

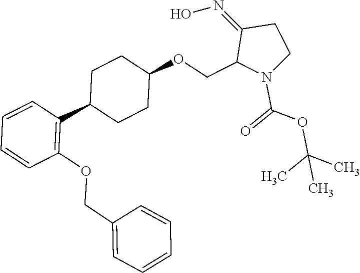 [SMILES: CC(C)(C)OC(=O)N1CC/C(=N/O)C1CO[C@H]1CC[C@@H](c2ccccc2OCc2ccccc2)CC1]

### tert-butyl (3E/Z)-3-(hydroxyimino)-2-({[(1s,4s)-4-[2-(benzyloxy)phenyl]cyclohexyl]oxy}methyl)pyrrolidine-1-carboxylate

A solution of N,N-diethylethanamine (8.0 mL, 57.4 mmol), hydroxylamine hydrochloride (1:1) (3.99 g, 57.4 mmol) and Intermediate 2 (92%, 10.00 g, 19.2 mmol) in ethanol (38.818 mL) was heated to 90° C. for 1 h. The reaction mixture was diluted with water (50 mL) and extracted with EtOAc (3×75 mL). The organic extracts were dried over magnesium sulfate and concentrated in vacuo to afford the title compound (10 g) as a yellow gum. [M+Na]⁺ m/z=517.3.

### Intermediate 4

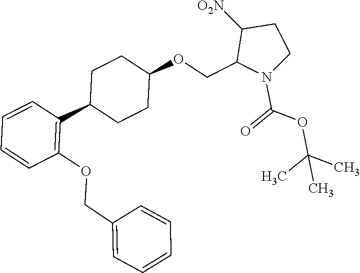 [SMILES: CC(C)(C)OC(=O)N1CCC([N+](=O)[O-])C1CO[C@H]1CC[C@@H](c2ccccc2OCc2ccccc2)CC1]

### tert-butyl 3-nitro-2-({[(1s,4s)-4-[2-(benzyloxy)phenyl]cyclohexyl]oxy}methyl)pyrrolidine-1-carboxylate

A solution of trifluoroacetic anhydride (7.0 mL, 50.5 mmol) in acetonitrile (36 mL) was added to a stirred solution of hydrogen peroxide-urea (1:1) (6.60 g, 70.2 mmol) in acetonitrile (36 mL) at 0° C. and the mixture was stirred at 0° C. for 2 h. The resulting solution was added dropwise to a mixture of intermediate 3 (10.00 g, 20.2 mmol) and sodium hydrogen carbonate (8.50 g, 0.101 mol) in Acetonitrile (36 mL) at 80° C. for 1 h. The reaction mixture was cooled to room temperature, quenched with sat. Na₂SO₃ (50 mL) and stirred for 10 min then extracted with EtOAc (2×100 mL). The combined organic extracts were dried over MgSO₄, filtered, and concentrated in vacuo to afford the crude material. The crude material was purified by silica gel column chromatography (0-40% EtOAc in heptane), to afford the title compounds (6.70 g) as a yellow gum. [M+H]⁺ m/z=511.3.

### Intermediate 5

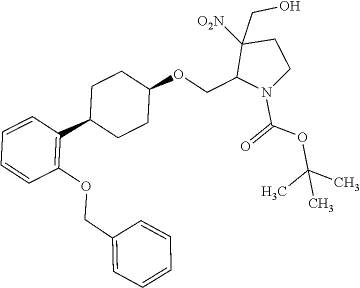 [SMILES: CC(C)(C)OC(=O)N1CCC(CO)([N+](=O)[O-])C1CO[C@H]1CC[C@@H](c2ccccc2OCc2ccccc2)CC1]

### tert-butyl-rel-(2R,3S)-3-(hydroxymethyl)-3-nitro-2-({[(1s,4s)-4-[2-(benzyloxy)phenyl]cyclohexyl]oxy}methyl)pyrrolidine-1-carboxylate

Formaldehyde (in water) (37%, 8.8 mL, 0.118 mol) was added to intermediate 5 (6.70 g, 13.1 mmol) and triethylamine (2.2 mL, 15.8 mmol) in THF (66 mL) at room temperature. The solution was heated to 70° C. for 18 h. After cooling the reaction mixture was diluted with water (50 mL) and extracted with EtOAc (3×75 mL). The combined organic extracts were washed with brine (50 mL), dried (MgSO₄), filtered and concentrated in vacuo. The crude material was purified by silica gel column chromatography (0-60% EtOAc in heptane), to afford the title compound (5.80 g) as a yellow solid. [M+H]⁺ m/z=541.4

### Intermediate 6

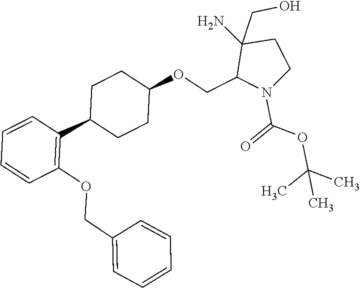 [SMILES: CC(C)(C)OC(=O)N1CCC(N)(CO)C1CO[C@H]1CC[C@@H](c2ccccc2OCc2ccccc2)CC1]

### tert-butyl-rel-(2R,3S)-3-amino-3-(hydroxymethyl)-2-({[(1s,4s)-4-[2-(benzyloxy)phenyl]cyclohexyl]oxy}methyl)pyrrolidine-1-carboxylate

A suspension of intermediate 5 (5.80 g, 10.7 mmol) and zinc (7.00 g, 0.107 mol) in acetic acid (49 mL) and ethanol (371 mL) was stirred for 12 h at room temperature. The reaction mixture was filtered through a pad of celite and washed with MeOH. The filtrate was neutralized with NaHCO₃, extracted with DCM (3×75 mL). The combined organic layers were dried (MgSO₄) and concentrated under vacuum to afford the title compound (5.49 g) as a yellow oil. [M+H]⁺ m/z 511.4.

### Intermediate 7

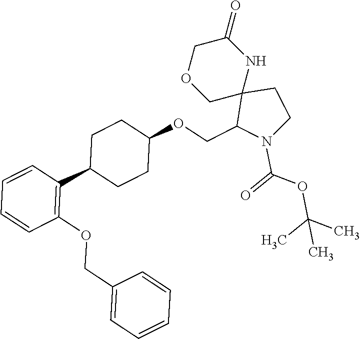 [SMILES: CC(C)(C)OC(=O)N1CCC2(COCC(=O)N2)C1CO[C@H]1CC[C@@H](c2ccccc2OCc2ccccc2)CC1]

### tert-butyl-rel-(1R,5S)-7-oxo-1-({[(1s,4s)-4-[2-(benzyloxy)phenyl]cyclohexyl]oxy}methyl)-9-oxa-2,6-diazaspiro[4.5]decane-2-carboxylate

To a solution of intermediate 6 (3.00 g, 5.87 mmol) in THF (25 mL) at 0° C. was added dipotassium carbonate (2.43 g, 17.6 mmol) then water (25 mL). To this mixture chloroacetyl chloride (0.65 mL, 8.15 mmol) was added dropwise at 0° C. The reaction was stirred for 1 h at 0° C. The mixture was quenched with water and extracted with DCM (3×50 mL) The combined organic extracts were washed with brine (40 mL), dried (MgSO₄), filtered and concentrated to give an oily residue. The intermediate was dissolved in DCM (53 mL) and IPA (82 mL), cooled to 0° C. potassium 2-methylpropan-2-olate (2.63 g, 23.4 mmol) was added and the reaction was stirred at 0° C. for 1 h. The reaction was quenched by addition of water (20 mL). The mixture was poured onto aqueous saturated NaHCO₃ (30 ml). After extraction with DCM (3×50 mL), the combined organic extracts were washed with brine (20 mL), dried (MgSO₄), filtered and concentrated to give the title compound (3.12 g) as a yellow solid [M+H]⁺ m/z 551.4.

### Intermediate 8

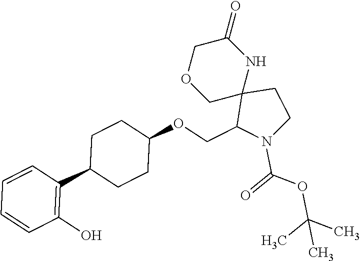 [SMILES: CC(C)(C)OC(=O)N1CCC2(COCC(=O)N2)C1CO[C@H]1CC[C@@H](c2ccccc2O)CC1]

### tert-butyl-rel-(1R,5S)-7-oxo-1-({[(1s,4s)-4-(2-hydroxyphenyl)cyclohexyl]oxy}methyl)-9-oxa-2,6-diazaspiro[4.5]decane-2-carboxylate

Intermediate 7 (1.00 g, 1.62 mmol) was dissolved in ethanol (77 mL) and the atmosphere was evacuated and backfilled with nitrogen 3 times. palladium on carbon (10%) (5.0%, 344 mg, 0.161 mmol) was added and the atmosphere was evacuated and backfilled with hydrogen 3 times. The reaction was stirred for 6 hours and then filtered through celite, washing with EtOAc, and concentrated in vacuo to afford the title compound (680 mg) as a white solid. [M+H]⁺ m/z 461.4

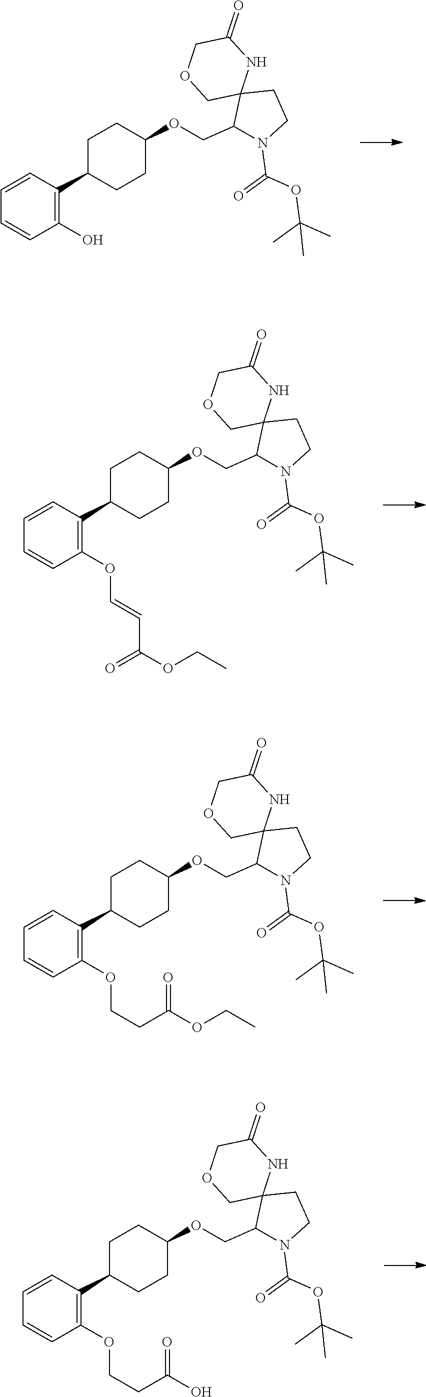 [SMILES: CC(C)(C)OC(=O)N1CCC2(COCC(=O)N2)C1CO[C@H]1CC[C@@H](c2ccccc2O)CC1 ; CCOC(=O)/C=C/Oc1ccccc1[C@H]1CC[C@@H](OCC2N(C(=O)OC(C)(C)C)CCC23COCC(=O)N3)CC1 ; CCOC(=O)CCOc1ccccc1[C@H]1CC[C@@H](OCC2N(C(=O)OC(C)(C)C)CCC23COCC(=O)N3)CC1 ; CC(C)(C)OC(=O)N1CCC2(COCC(=O)N2)C1CO[C@H]1CC[C@@H](c2ccccc2OCCC(=O)O)CC1]

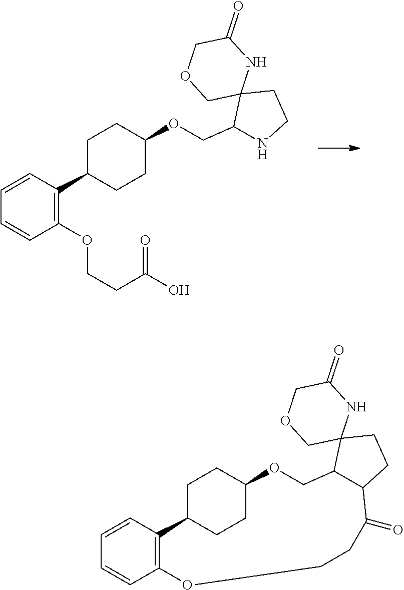 [SMILES: O=C(O)CCOc1ccccc1[C@H]1CC[C@@H](OCC2NCCC23COCC(=O)N3)CC1 ; O=C1COCC2(CCC3C(=O)CCOc4ccccc4[C@H]4CC[C@H](CC4)OCC32)N1]

### Intermediate 9

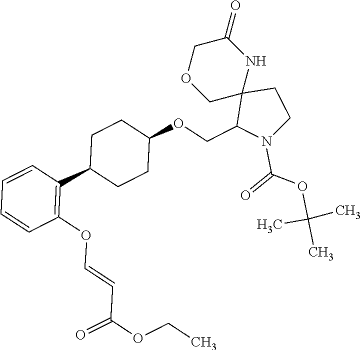 [SMILES: CCOC(=O)/C=C/Oc1ccccc1[C@H]1CC[C@@H](OCC2N(C(=O)OC(C)(C)C)CCC23COCC(=O)N3)CC1]

### tert-butyl-rel-(1R,5S)-7-oxo-1-({[(1s,4s)-4-(2-{[3-ethoxy-3-oxoprop-1-en-1-yl]oxy}phenyl)cyclohexyl]oxy}methyl)-9-oxa-2,6-diazaspiro[4.5]decane-2-carboxylate

To a solution of ethyl prop-2-ynoate (98%, 44 mg, 0.44 mmol) and 1,4-diazabicyclo[2.2.2]octane (4.1 mg, 0.0366 mmol) in THF (1.68 mL) was added intermediate 8 (84%, 200 mg, 0.365 mmol) in THF (8.4 mL) at ° C. under nitrogen atmosphere and the solution was stirred at room temperature for 3 hours. The reaction mixture was quenched with water (10 mL) and extracted with EtOAc (2×15 mL). The organic phase was washed with brine, dried over MgSO₄, and concentrated in vacuo to afford the title compound (160 mg) as a yellow oil. [M+H]⁺ m/z 559.4.

### Intermediate 10

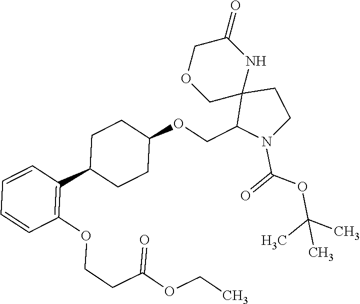 [SMILES: CCOC(=O)CCOc1ccccc1[C@H]1CC[C@@H](OCC2N(C(=O)OC(C)(C)C)CCC23COCC(=O)N3)CC1]

### tert-butyl-rel-(1R,5S)-7-oxo-1-({[(1s,4s)-4-[2-(3-ethoxy-3-oxopropoxy)phenyl]cyclohexyl]oxy}methyl)-9-oxa-2,6-diazaspiro[4.5]decane-2-carboxylate

Intermediate 9 (64%, 160 mg, 0.183 mmol) and ammonium formate (114 mg, 1.84 mmol) were dissolved in IPA (2.6 mL) then palladium (2+) dihydroxide (20%, 129 mg, 0.184 mmol) was added at room temperature under nitrogen. The reaction was stirred at 80° C. for 5 hours, cool to room temperature, filtered through celite, washed with IPA, and concentrated in vacuo to afford the title compound (120 mg) as a yellow oil. [M+H]⁺ m/z 561.4

### Intermediate 11

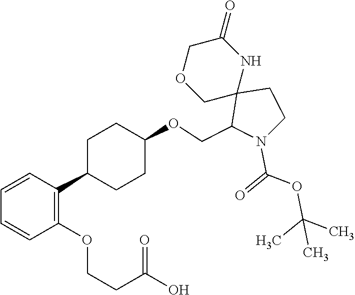 [SMILES: CC(C)(C)OC(=O)N1CCC2(COCC(=O)N2)C1CO[C@H]1CC[C@@H](c2ccccc2OCCC(=O)O)CC1]

### 3-{2-[(1s,4s)-4-{[rel-(1R,5S)-2-[(tert-butoxy)carbonyl]-7-oxo-9-oxa-2,6-diazaspiro[4.5]decan-1-yl]methoxy}cyclohexyl]phenoxy}propanoic acid

To a solution of intermediate 10 (72%, 120 mg, 0.154 mmol) in THF (4.4 mL) and water (1.25 mL), aqueous 2 M lithium hydroxide (0.77 mL, 1.54 mmol) was added at room temperature and mixture was stirred at room temperature for 18 hours. The mixture was diluted with water (5 mL) and neutralized to pH 7 with aq. 1M HCL then extracted with 10% MeOH in DCM (3×10 mL). The organic layer was dried over MgSO₄ and concentrated in vacuo to afford the title (110 mg) as a colorless oil. [M+H]⁺ m/z: 533.4

### Intermediate 12

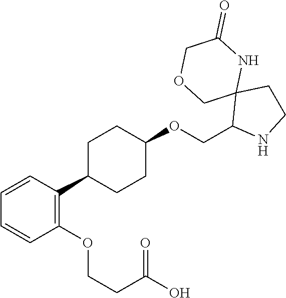 [SMILES: O=C(O)CCOc1ccccc1[C@H]1CC[C@@H](OCC2NCCC23COCC(=O)N3)CC1]

### 3-{2-[(1s,4s)-4-{[rel-(1R,5S)-7-oxo-9-oxa-2,6-diazaspiro[4.5]decan-1-yl]methoxy}cyclohexyl]phenoxy}propanoic acid

4 M hydrogen chloride in dioxane (0.20 mL, 0.805 mmol) was added to intermediate 11 (50 mg, 0.109 mmol) and the reaction was stirred at room temperature for 30 min. The aqueous phase was neutralized with a few drops of aq. 1M K₂CO₃, washed with DCM (2×2 mL) and concentrated in vacuo to afford the title compound (60 mg) as a yellow solid. [M+H]⁺ m/z: 433.3

### Example 1

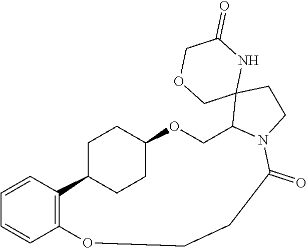 [SMILES: O=C1COCC2(CCN3C(=O)CCOc4ccccc4[C@H]4CC[C@H](CC4)OCC32)N1]

### rel-(1's,3S,16′R,19′s)-8′,18′-dioxa-12′-azaspiro[morpholine-3,15′-tetracyclo[17.2.2.0^{2,7}0.0^{12,16}]tricosane]-2′(7′),3′,5′-triene-5,11′-dione

To a stirred solution of HATU (49 mg, 0.129 mmol) and DIPEA (45 μL, 0.255 mmol) in acetonitrile (40 mL) was added intermediate 12 (62%, 60 mg, 0.0860 mmol) in acetonitrile (3.6 mL) over 2h using a syringe pump. The resulting solution was stirred for one hours. The reaction mixture was concentrated in vacuo to afford the crude material. The mixture was diluted in water and extracted with DCM (3×5 mL) The combined organic extracts were dried (MgSO₄), filtered and concentrated to afford the crude as a solid. The crude material was purified by basic reverse phase column chromatography (10-55% acetonitrile in water (0.1% ammonia), to afford the title compound (33 mg) as a white solid.

LCMS (Method A): [M+H]⁺ m/z 415.3, RT 2.87 minutes

¹H NMR (500 MHZ, CDCl₃) δ 7.20-7.11 (m, 2H), 7.09 (dd, J=7.5, 1.7 Hz, 1H), 7.01 (d, J=7.8 Hz, 1H), 6.93-6.86 (m, 1H), 6.42 (s, 1H), 4.57-4.32 (m, 2H), 4.29 (d, J=2.4 Hz, 1H), 4.29-4.10 (m, 3H), 4.07-3.95 (m, 1H), 3.83 (s, 1H), 3.78 (d, J=11.6 Hz, 1H), 3.65-3.53 (m, 2H), 3.30 (d, J=9.4 Hz, 1H), 3.18-3.06 (m, 1H), 2.55-2.36 (m, 3H), 2.37-2.25 (m, 1H), 2.22-2.06 (m, 3H), 1.88-1.78 (m, 1H), 1.60-1.50 (m, 1H), 1.51-1.45 (m, 1H), 1.43-1.30 (m, 1H).

### Examples 1a and 1b

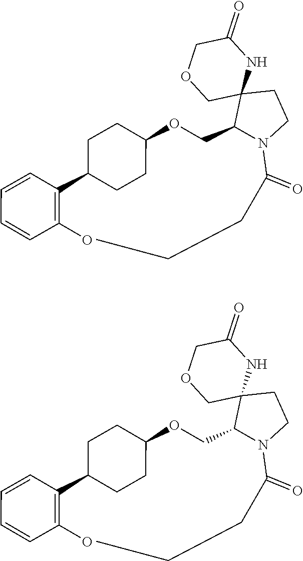 [SMILES: CCCC(=O)N1CC[C@@]2(COCC(=O)N2)[C@@H]1CO[C@H]1CC[C@@H](c2ccccc2O)CC1 ; CCCC(=O)N1CC[C@]2(COCC(=O)N2)[C@H]1CO[C@H]1CC[C@@H](c2ccccc2O)CC1]

### Example 1a: (1's,3S,16′R,19′s)-8′,18′-dioxa-12′-azaspiro[morpholine-3,15′-tetracyclo[17.2.2.0^{2,7}0.0^{12,16}]tricosane]-2′(7′),3′,5′-triene-5,11′-dione

### Example 1b: (1′s,3R,16′S,19′s)-8′,18′-dioxa-12′-azaspiro[morpholine-3,15′-tetracyclo[17.2.2.0^{2,7}0.0^{12,16}]tricosane]-2′(7′),3′,5′-triene-5,11′-dione

Example 1 (33 mg) was subjected to chiral preparative purification using Waters 600 eluting with 30/70% v/v n-Hexane/(ethanol/methanol 1:1+0.1% isopropylamine), Chiralpak IC (25×2.0 cm), 5 μm, flow rate 17 mL/minutes to afford the title compounds (Peak 1, 11.1 mg, 100% ee; and Peak 2, 10.9 mg, 100% ee).

### Example 1a: Peak 1 (Stereochemistry Arbitrarily Assigned at Pyrrolidine)

LCMS (Method C): [M+H]⁺ m/z 415.3, RT 1.00 minutes.

Chiral analysis (Chiralpak IC, 25×0.46 cm, 5 μm, 30:70 n-Hexane: (ethanol/methanol 1:1 +0.1% isopropylamine)): RT 9.2 minutes

¹H NMR (500 MHZ, CDCl₃) δ 7.21-7.13 (m, 1H), 7.09 (dd, J=7.5, 1.6 Hz, 1H), 7.01 (d, J=8.1 Hz, 1H), 6.89 (td, J=7.4, 1.0 Hz, 1H), 6.26 (s, 1H), 4.47-4.44 (m, 1H), 4.45-4.42 (m, 1H), 4.29 (d, J=2.2 Hz, 1H), 4.25 (d, J=16.9 Hz, 1H), 4.26-4.21 (m, 1H), 4.18 (d, J=16.9 Hz, 1H), 4.01 (td, J=10.0, 3.2 Hz, 1H), 3.83 (br s, 1H), 3.78 (d, J=11.7 Hz, 1H), 3.59 (d, J=11.7 Hz, 1H), 3.59-3.53 (m, 1H), 3.30 (d, J=9.5 Hz, 1H), 3.12 (ddd, J=14.2, 12.2, 4.2 Hz, 1H), 2.50-2.40 (m, 2H), 2.42-2.36 (m, 1H), 2.30 (qd, J=12.8, 3.7 Hz, 1H), 2.22-2.16 (m, 1H), 2.19-2.11 (m, 1H), 2.15-2.08 (m, 1H), 1.84-1.77 (m, 1H), 1.60-1.51 (m, 1H), 1.48 (br d, J=12.2 Hz, 1H), 1.43-1.30 (m, 2H).

### Example 1b: Peak 2 (Stereochemistry Arbitrarily Assigned at Pyrrolidine)

LCMS (Method C): [M+H]⁺ m/z 415.3, RT 1.00 minutes.

Chiral analysis (Chiralpak IC, 25×0.46 cm, 5 μm, 30:70 n-Hexane: (ethanol/methanol 1:1 +0.1% isopropylamine)): RT 15.9 minutes

¹H NMR (500 MHZ, CDCl₃) δ 7.21-7.13 (m, 1H), 7.09 (dd, J=7.5, 1.6 Hz, 1H), 7.01 (d, J=8.1 Hz, 1H), 6.89 (td, J=7.4, 1.0 Hz, 1H), 6.26 (s, 1H), 4.47-4.44 (m, 1H), 4.45-4.42 (m, 1H), 4.29 (d, J=2.2 Hz, 1H), 4.25 (d, J=16.9 Hz, 1H), 4.26-4.21 (m, 1H), 4.18 (d, J=16.9 Hz, 1H), 4.01 (td, J=10.0, 3.2 Hz, 1H), 3.83 (br s, 1H), 3.78 (d, J=11.7 Hz, 1H), 3.59 (d, J=11.7 Hz, 1H), 3.59-3.53 (m, 1H), 3.30 (d, J=9.5 Hz, 1H), 3.12 (ddd, J=14.2, 12.2, 4.2 Hz, 1H), 2.50-2.40 (m, 2H), 2.42-2.36 (m, 1H), 2.30 (qd, J=12.8, 3.7 Hz, 1H), 2.22-2.16 (m, 1H), 2.19-2.11 (m, 1H), 2.15-2.08 (m, 1H), 1.84-1.77 (m, 1H), 1.60-1.51 (m, 1H), 1.48 (br d, J=12.2 Hz, 1H), 1.43-1.30 (m, 2H).

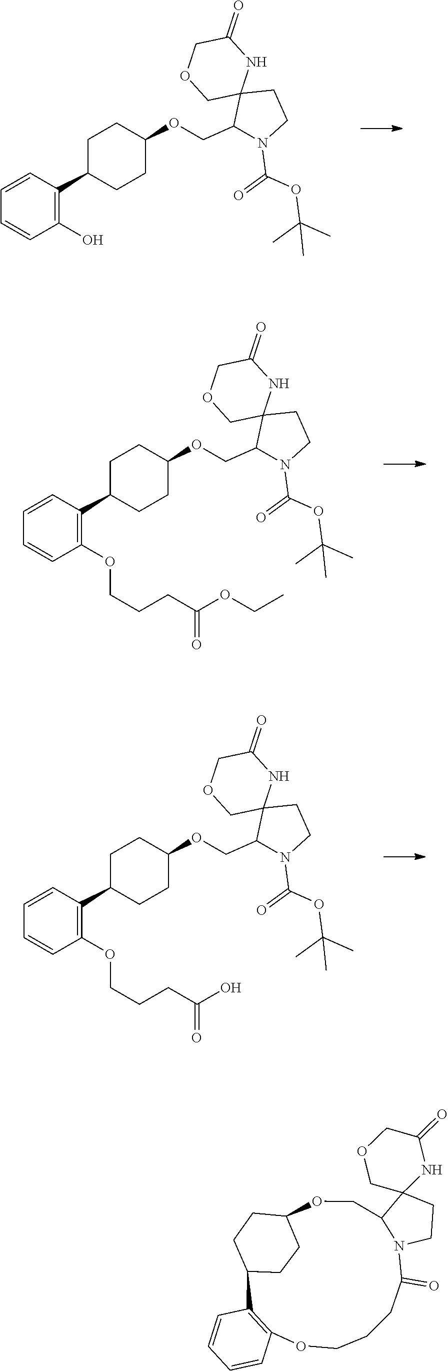 [SMILES: CC(C)(C)OC(=O)N1CCC2(COCC(=O)N2)C1CO[C@H]1CC[C@@H](c2ccccc2O)CC1 ; CCOC(=O)CCCOc1ccccc1[C@H]1CC[C@@H](OCC2N(C(=O)OC(C)(C)C)CCC23COCC(=O)N3)CC1 ; CC(C)(C)OC(=O)N1CCC2(COCC(=O)N2)C1CO[C@H]1CC[C@@H](c2ccccc2OCCCC(=O)O)CC1 ; O=C1COCC2(CCN3C(=O)CCCOc4ccccc4[C@H]4CC[C@H](CC4)OCC32)N1]

### Intermediate 13

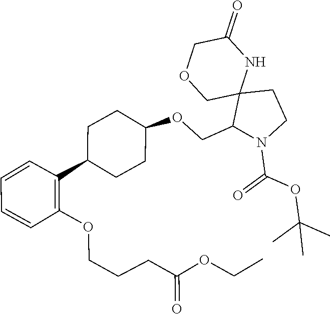 [SMILES: CCOC(=O)CCCOc1ccccc1[C@H]1CC[C@@H](OCC2N(C(=O)OC(C)(C)C)CCC23COCC(=O)N3)CC1]

### tert-butyl-rel-(1R,5S)-7-oxo-1-({[(1s,4s)-4-[2-(4-ethoxy-4-oxobutoxy)phenyl]cyclohexyl]oxy}methyl)-9-oxa-2,6-diazaspiro[4.5]decane-2-carboxylate

To a solution of intermediate 8 (200 mg, 0.434 mmol) and ethyl 4-bromobutanoate (0.12 mL, 0.866 mmol) in acetone (2.7 mL) was added dipotassium carbonate (180 mg, 1.30 mmol) and the solution heated to 50° C. for 24 hours. The solids were removed by filtration, and the filtrate was concentrated in vacuo, suspended in water (5 mL) and extracted with DCM (3×5 mL). The solvent was removed in vacuo, to afford the title compound (202 mg) as a colorless oil. [M+H]⁺ m/z 575.4

### Intermediate 14

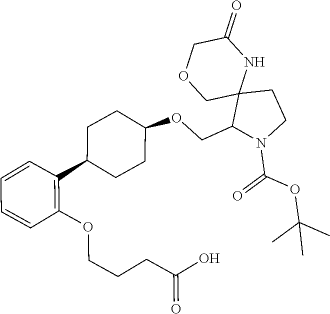 [SMILES: CC(C)(C)OC(=O)N1CCC2(COCC(=O)N2)C1CO[C@H]1CC[C@@H](c2ccccc2OCCCC(=O)O)CC1]

### 4-{2-[(1s,4s)-4-{[rel-(1R,5S)-2-[(tert-butoxy)carbonyl]-7-oxo-9-oxa-2,6-diazaspiro[4.5]decan-1-yl]methoxy}cyclohexyl]phenoxy}butanoic acid

To a solution of intermediate 13 (64%, 202 mg, 0.225 mmol) in THF (6.4 mL) and water (1.8 mL), aqueous 2 M lithium hydroxide (1.1 mL, 2.25 mmol) was added at room temperature. The mixture was stirred at room temperature overnight. The mixture was diluted with water (5 mL) and neutralized with aq. 1M HCl to pH 7, and the aqueous phase extracted with 10% methanol in DCM (3×10 mL). The organic layer was dried (MgSO₄) and concentrated in vacuo, to afford the title compound (200 mg) as a colorless oil. [M+H]⁺ m/z: 547.4

### Intermediate 15

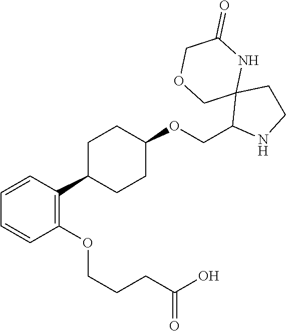 [SMILES: O=C(O)CCCOc1ccccc1[C@H]1CC[C@@H](OCC2NCCC23COCC(=O)N3)CC1]

### 4-{2-[(1s,4s)-4-{[rel-(1R,5S)-7-oxo-9-oxa-2,6-diazaspiro[4.5]decan-1-yl]methoxy}cyclohexyl]phenoxy}butanoic acid

4 M hydrogen chloride (0.45 mL, 1.80 mmol) was added to intermediate 14 (50 mg, 0.109 mmol) at room temperature and the reaction was stirred for 30 min. The aqueous phase was washed with DCM (2×2 mL) and concentrated in vacuo, to afford the title compound (80 mg) as a yellow solid. [M+H]⁺ 447.4

### Example 2

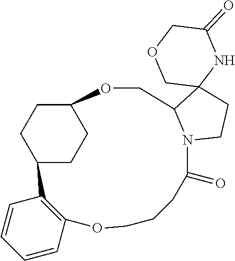 [SMILES: O=C1COCC2(CCN3C(=O)CCCOc4ccccc4[C@H]4CC[C@H](CC4)OCC32)N1]

### rel-(1′s,3S, 17′R,20′s)-8′,19′-dioxa-13′-azaspiro[morpholine-3, 16′-tetracyclo [18.2.2.0^{2,7}0.0^{13,17}]tetracosane]-2′(7′),3′,5′-triene-5,12′-dione

To a stirred solution of HATU (75 mg, 0.199 mmol) and DIPEA (68 μL, 0.391 mmol) in acetonitrile (63.5 mL) was added intermediate 15 (74%, 80 mg, 0.133 mmol) in acetonitrile (2.6 mL) over 2h using a syringe pump. The resulting solution was stirred for one hours. The reaction mixture was concentrated in vacuo to afford the crude material. The mixture was diluted with water and extracted with DCM (3×5 mL) The combined organic extracts were dried (MgSO₄), filtered, and concentrated to give a solid residue. The crude material was purified by basic reverse phase column chromatography (10-50% acetonitrile in water (0.1% ammonia), to afford the title compound (31 mg) as a white solid.

LCMS (Method A): [M+H]⁺ m/z 429.3, RT 2.98 minutes

¹H NMR (500 MHZ, CDCl₃) δ 7.77 (s, 1H), 7.19-7.12 (m, 1H), 7.08 (dd, J=7.4, 1.6 Hz, 1H), 6.86 (d, J=8.6 Hz, 1H), 6.83 (d, J=7.3 Hz, 1H), 4.38 (dd, J=10.7, 2.7 Hz, 1H), 4.28 (d, J=17.1 Hz, 1H), 4.23-4.18 (m, 1H), 4.13 (d, J=17.1 Hz, 1H), 4.06-3.92 (m, 1H), 3.86-3.77 (m, 1H), 3.77-3.72 (m, 1H), 3.73-3.69 (m, 1H), 3.64 (dd, J=9.4, 2.9 Hz, 1H), 3.58 (s, 1H), 3.57-3.50 (m, 1H), 3.49-3.45 (m, 1H), 3.39 (d, J=11.8 Hz, 1H), 2.56-2.48 (m, 3H), 2.40 (ddd, J=16.4, 9.6, 5.1 Hz, 1H), 2.26-2.12 (m, 1H), 2.11-2.04 (m, 2H), 2.03-1.89 (m, 3H), 1.51-1.40 (m, 4H).

### Example 3

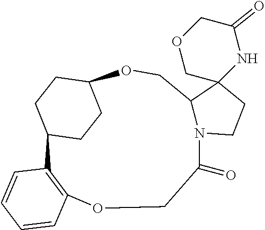 [SMILES: O=C1COCC2(CCN3C(=O)COc4ccccc4[C@H]4CC[C@H](CC4)OCC32)N1]

### rel-(1′s,3S, 15′R,18′s)-8′,17′-dioxa-11′-azaspiro[morpholine-3,14′-tetracyclo[16.2.2.0^{2,7}0.0^{11,15}]docosane]-2′(7′),3′,5′-triene-5,10′-dione

Example 3 was prepared using intermediate 8 and following the procedure described for example 2 to afford the title compound (2.4 mg) as an off-white solid.

LCMS (Method A): [M+H]⁺ m/z 401.2, RT 2.88 minutes

¹H NMR (500 MHZ, CDCl₃) δ 7.23-7.16 (m, 1H), 7.10 (dd, J=7.5, 1.7 Hz, 1H), 6.93 (td, J=7.4, 1.1 Hz, 1H), 6.78 (dd, J=8.0, 0.9 Hz, 1H), 6.38 (s, 1H), 5.00 (d, J=10.4 Hz, 1H), 4.42-4.37 (m, 1H), 4.36 (dd, J=10.2, 3.4 Hz, 1H), 4.33-4.25 (m, 3H), 4.20 (d, J=16.7 Hz, 1H), 3.87 (s, 1H), 3.83 (d, J=11.7 Hz, 1H), 3.64 (d, J=11.7 Hz, 1H), 3.62-3.53 (m, 1H), 3.34 (d, J=9.8 Hz, 1H), 2.62-2.54 (m, 1H), 2.54-2.48 (m, 1H), 2.49-2.39 (m, 1H), 2.21-2.12 (m, 2H), 2.11-2.00 (m, 1H), 1.87-1.80 (m, 1H), 1.58-1.49 (m, 2H), 1.44-1.32 (m, 2H).

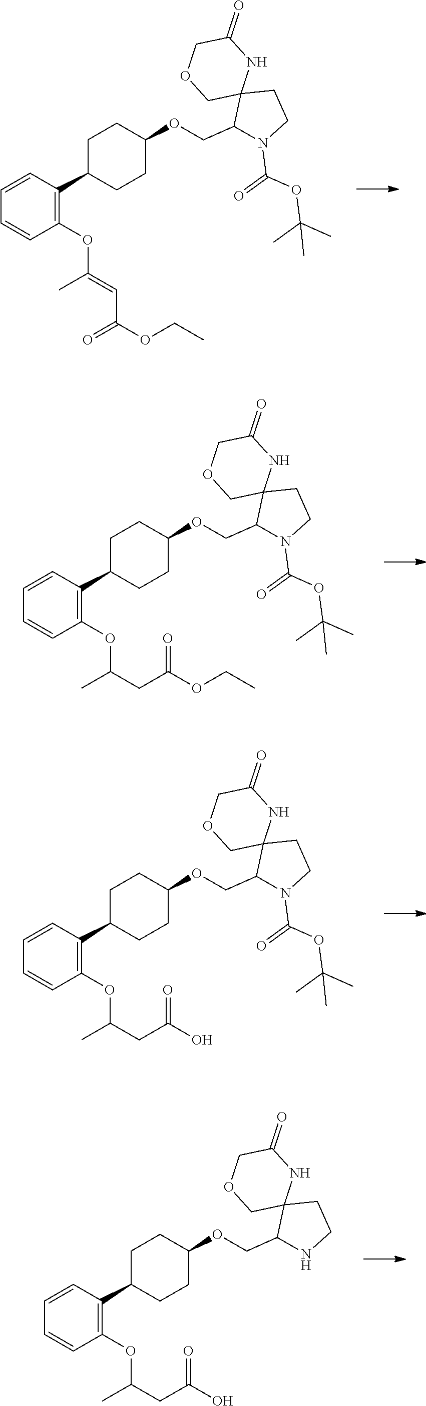 [SMILES: CCOC(=O)/C=C(\C)Oc1ccccc1[C@H]1CC[C@@H](OCC2N(C(=O)OC(C)(C)C)CCC23COCC(=O)N3)CC1 ; CCOC(=O)CC(C)Oc1ccccc1[C@H]1CC[C@@H](OCC2N(C(=O)OC(C)(C)C)CCC23COCC(=O)N3)CC1 ; CC(CC(=O)O)Oc1ccccc1[C@H]1CC[C@@H](OCC2N(C(=O)OC(C)(C)C)CCC23COCC(=O)N3)CC1 ; CC(CC(=O)O)Oc1ccccc1[C@H]1CC[C@@H](OCC2NCCC23COCC(=O)N3)CC1]

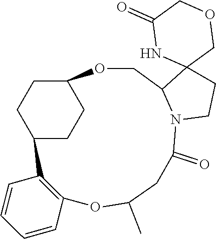 [SMILES: CC1CC(=O)N2CCC3(COCC(=O)N3)C2CO[C@H]2CC[C@H](CC2)c2ccccc2O1]

### Intermediate 16

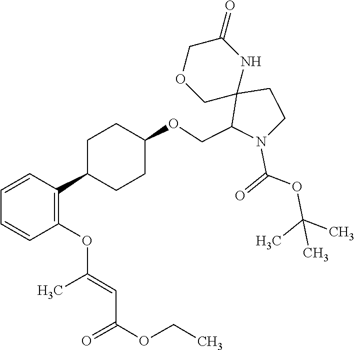 [SMILES: CCOC(=O)/C=C(\C)Oc1ccccc1[C@H]1CC[C@@H](OCC2N(C(=O)OC(C)(C)C)CCC23COCC(=O)N3)CC1]

### tert-butyl-rel-(1R,5S)-7-oxo-1-({[(1s,4s)-4-(2-{[4-ethoxy-4-oxobut-2-en-2-yl]oxy}phenyl)cyclohexyl]oxy}methyl)-9-oxa-2,6-diazaspiro[4.5]decane-2-carboxylate

To a solution of Intermediate 8 (84%, 200 mg, 0.365 mmol) in acetonitrile (3 mL) at room temperature under nitrogen was added 1,4-diazabicyclo[2.2.2]octane (41 mg, 0.365 mmol) and the solution heated to 70° C. ethyl but-2-ynoate (0.047 mL, 0.401 mmol) was added to the reaction and the solution stirred for 16 hours at 70° C. The reaction mixture was quenched with water (10 ml) and extracted with EtOAc (2×15 ml). The organic phase was washed with brine, dried over MgSO₄, and concentrated in vacuo. The crude material was purified by column chromatography (0-100% EtOAc in heptane), to afford to afford the title compound (198 mg) as a yellow oil. [M+H]⁺ m/z 573.4

### Intermediate 17

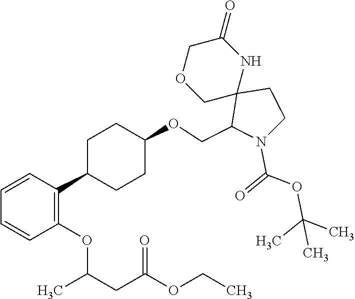 [SMILES: CCOC(=O)CC(C)Oc1ccccc1[C@H]1CC[C@@H](OCC2N(C(=O)OC(C)(C)C)CCC23COCC(=O)N3)CC1]

### tert-butyl-rel-(1R,5S)-7-oxo-1-({[(1s,4s)-4-{2-[(4-ethoxy-4-oxobutan-2-yl)oxy]phenyl}cyclohexyl]oxy}methyl)-9-oxa-2,6-diazaspiro[4.5]decane-2-carboxylate

Intermediate 16 (89%, 198 mg, 0.308 mmol) and ammonium formate (191 mg, 3.08 mmol) were dissolved in IPA (4.4 mL). palladium (2+) dihydroxide (20%, 216 mg, 0.308 mmol) was added at room temperature under nitrogen. The reaction was stirred at 80° C. for 5 hours, then filtered through celite, washing with isopropanol and concentrated in vacuo to afford the title compound (168 mg) as a yellow oil [M+H]⁺ m/z 575.3

### Intermediate 18

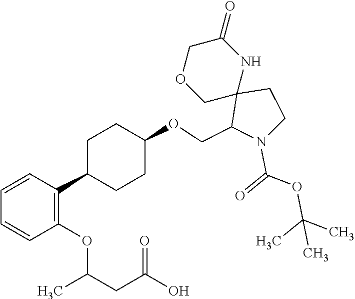 [SMILES: CC(CC(=O)O)Oc1ccccc1[C@H]1CC[C@@H](OCC2N(C(=O)OC(C)(C)C)CCC23COCC(=O)N3)CC1]

### 3-{2-[(1s,4s)-4-{[rel-(1R,5S)-2-[(tert-butoxy)carbonyl]-7-oxo-9-oxa-2,6-diazaspiro[4.5]decan-1-yl]methoxy}cyclohexyl]phenoxy}butanoic acid

To a solution of Intermediate 17 (76%, 168 mg, 0.222 mmol) in THF (6.4 mL) and Water (1.8 mL), aqueous 2 M lithium hydroxide (5.6 mL, 11.1 mmol) was added at room temperature. The mixture was stirred at room temperature for 18 hours. The reaction mixture was diluted with water (5 ml), washed with DCM (2×3 mL) and neutralized to pH 7 with aq. 1M HCL and the aqueous layer was extracted with 10% MeOH in DCM (3×10 mL). The organic layer was dried (MgSO₄) and concentrated in vacuo to afford the title compound (90 mg) as a colorless oil. [M+H]⁺ m/z 547.4

### Intermediate 19

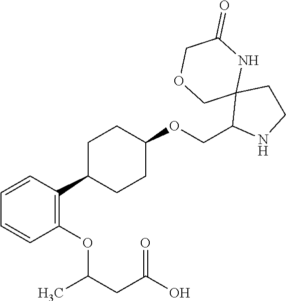 [SMILES: CC(CC(=O)O)Oc1ccccc1[C@H]1CC[C@@H](OCC2NCCC23COCC(=O)N3)CC1]

### 3-{2-[(1s,4s)-4-{[rel-(1R,5S)-7-oxo-9-oxa-2,6-diazaspiro[4.5]decan-1-yl]methoxy}cyclohexyl]phenoxy}butanoic acid

4 M hydrogen chloride in dioxane (2.0 mL, 7.82 mmol) was added to Intermediate 18 (90 mg, 0.109 mmol) at room temperature and the reaction was stirred for 30 min. The aqueous phase was washed with DCM (2×2 mL) and concentrated in vacuo to afford the title compound (74 mg) as a yellow solid. [M+H]⁺ m/z 447.3

### Example 4

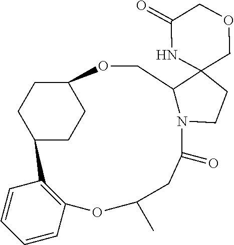 [SMILES: CC1CC(=O)N2CCC3(COCC(=O)N3)C2CO[C@H]2CC[C@H](CC2)c2ccccc2O1]

### rel-(1′s,3S,16′R,19′s)-9′-methyl-8′,18′-dioxa-12′-azaspiro[morpholine-3,15′-tetracyclo[17.2.2.0^{2,7}0.0^{12,16}]tricosane]-2′(7′),3′,5′-triene-5,11′-dione

To a stirred solution of HATU (76 mg, 0.200 mmol) and DIPEA (69 μL, 0.395 mmol) in acetonitrile (33 mL) was added Intermediate 19 (80%, 74 mg, 0.133 mmol) in acetonitrile (2.9 mL) over 2 hours using a syringe pump. The resulting solution was stirred for one hours. The reaction mixture was concentrated in vacuo to afford the crude material. The mixture was diluted in water and extracted with DCM (3×5 mL). The combined organic extracts were dried (MgSO₄), filtered and concentrated to give a solid residue. The crude material was purified by basic reverse phase column chromatography (10-60% acetonitrile in water (0.1% ammonia), to afford the title compound (26 mg) as a white solid. [M+H]⁺ m/z 429.3

LCMS (Method A): [M+H]⁺ m/z 429.3, RT 2.98 minutes

¹H NMR (400 MHZ, CDCl₃) δ 7.08-6.98 (m, 2H), 6.88-6.76 (m, 2H), 6.42 (s, 1H), 4.69-4.53 (m, 1H), 4.40 (dd, J=9.6, 3.3 Hz, 1H), 4.20-4.06 (m, 3H), 3.74 (s, 1H), 3.73-3.64 (m, 1H), 3.52 (d, J=11.7 Hz, 1H), 3.24-3.16 (m, 1H), 2.99-2.83 (m, 1H), 2.46-2.33 (m, 3H), 2.32-2.21 (m, 2H), 2.17-2.07 (m, 2H), 2.06-1.95 (m, 2H), 1.78-1.68 (m, 3H), 1.41-1.33 (m, 5H).

### Examples 4a, 4b, 4c and 4d

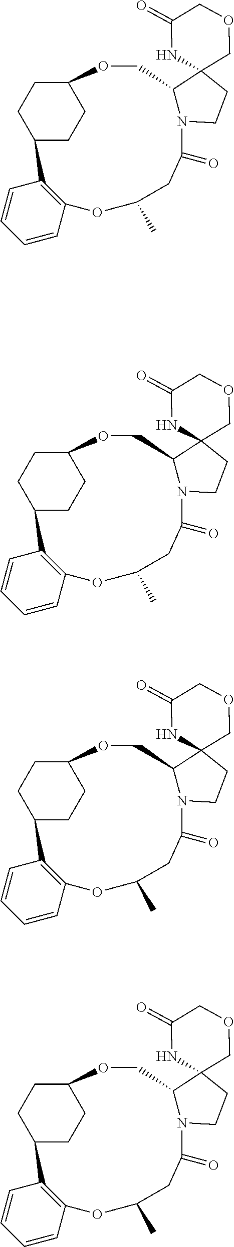 [SMILES: C[C@H]1CC(=O)N2CC[C@]3(COCC(=O)N3)[C@H]2CO[C@H]2CC[C@H](CC2)c2ccccc2O1 ; C[C@H]1CC(=O)N2CC[C@@]3(COCC(=O)N3)[C@@H]2CO[C@H]2CC[C@H](CC2)c2ccccc2O1 ; C[C@@H]1CC(=O)N2CC[C@@]3(COCC(=O)N3)[C@@H]2CO[C@H]2CC[C@H](CC2)c2ccccc2O1 ; C[C@@H]1CC(=O)N2CC[C@]3(COCC(=O)N3)[C@H]2CO[C@H]2CC[C@H](CC2)c2ccccc2O1]

### Example 4a: (1′s,3R,9′S,16′S,19′s)-9′-methyl-8′,18′-dioxa-12′-azaspiro[morpholine-3,15′-tetracyclo[17.2.2.0^{2,7}0.0^{12,16}]tricosane]-2′(7′),3′,5′-triene-5,11′-dione

### Example 4b: (1′s, 3S,9′S,16′R,19′s)-9′-methyl-8′,18′-dioxa-12′-azaspiro[morpholine-3,15′-tetracyclo[17.2.2.0^{2,7}0.0^{12,16}]tricosane]-2′(7′),3′,5′-triene-5,11′-dione

### Example 4c: (1′s,3S,9′R,16′R,19′s)-9′-methyl-8′,18′-dioxa-12′-azaspiro[morpholine-3,15′-tetracyclo[17.2.2.0^{2,7}0.0^{12,16}]tricosane]-2′(7′),3′,5′-triene-5,11′-dione

### Example 4d: (1′s,3R,9′R,16′S,19′s)-9′-methyl-8′,18′-dioxa-12′-azaspiro[morpholine-3,15′-tetracyclo[17.2.2.0^{2,7}0.0^{12,16}]tricosane]-2′(7′), 3′,5′-triene-5,11′-dione

Example 4 (24 mg) was subjected to chiral preparative purification using Waters 600 eluting with 50/50% v/v n-Hexane/ethanol+0.1% isopropylamine, Chiralpak AD-H (25×2.0 cm), 5 μm, flow rate 17 mL/minutes to afford the title compounds (Peak 1, 1 mg; Peak 2, 3.7 mg, 99% ee; Peak 3, 3.2 mg; Peak 4, 4.5 mg, 100% ee).

### Example 4a: Peak 1 (Stereochemistry Arbitrarily Assigned at Pyrrolidine and Methyl Center)

LCMS (Method C): [M+H]⁺ m/z 429.3, RT 0.96, 0.99 minutes.

Chiral analysis (Chiralpak AD-H, 25×0.46 cm, 5 μm, 50:50 n-Hexane: (ethanol+0.1% isopropylamine): RT 9.0 minutes

¹H NMR (400 MHZ, CDCl₃) δ 7.21-7.14 (m, 1H), 7.09 (d, J=7.5 Hz, 1H), 7.01 (d, J=8.1 Hz, 1H), 6.91-6.85 (m, 1H), 6.22 (s, 1H), 5.09-4.97 (m, 1H), 4.35 (br d, J=9.4 Hz, 1H), 4.32 (s, 1H), 4.29-4.12 (m, 2H), 4.03 (br t, J=10.0 Hz, 1H), 3.83 (br s, 1H), 3.80-3.74 (m, 1H), 3.56 (d, J=11.6 Hz, 1H), 3.55-3.46 (m, 1H), 3.37-3.31 (m, 1H), 3.24 (dd, J=13.6, 5.3 Hz, 1H), 2.57-2.41 (m, 2H), 2.34 (d, J=13.6 Hz, 1H), 2.15-2.00 (m, 1H), 1.44 (d, J=6.4 Hz, 3H), 2.25-1.15 (m, 8H).

### Example 4b: Peak 2 (Stereochemistry Arbitrarily Assigned at Pyrrolidine and Methyl Center))

LCMS (Method C): [M+H]⁺ m/z 429.3, RT 0.97 minutes.

Chiral analysis (Chiralpak AD-H, 25×0.46 cm, 5 μm, 50:50 n-Hexane: (ethanol+0.1% isopropylamine): RT 18.4 minutes

¹H NMR (400 MHZ, CDCl₃) δ 7.16-7.05 (m, 2H), 6.96-6.86 (m, 2H), 6.47-6.36 (m, 1H), 4.75-4.63 (m, 1H), 4.49 (dd, J=9.6, 3.1 Hz, 1H), 4.29-4.15 (m, 3H), 4.09 (td, J=9.8, 3.9 Hz, 1H), 3.82 (br s, 1H), 3.75 (d, J=11.6 Hz, 1H), 3.61 (d, J=11.6 Hz, 1H), 3.58-3.48 (m, 1H), 3.28 (d, J=9.6 Hz, 1H), 2.98 (dd, J=14.5, 9.9 Hz, 1H), 2.54-2.41 (m, 2H), 2.40-2.27 (m, 2H), 2.24-2.16 (m, 1H), 2.09 (td, J=8.3, 3.7 Hz, 1H), 2.08-1.96 (m, 1H), 1.82 (br d, J=13.6 Hz, 1H), 1.59-1.47 (m, 1H), 1.45 (d, J=5.9 Hz, 3H), 1.50-1.39 (m, 2H), 1.42-1.23 (m, 1H).

### Example 4c: Peak 3 (Stereochemistry Arbitrarily Assigned at Pyrrolidine and Methyl Center))

LCMS (Method C): [M+H]⁺ m/z 429.3, RT 0.96, 0.99 minutes.

Chiral analysis (Chiralpak AD-H, 25×0.46 cm, 5 μm, 50:50 n-Hexane: (ethanol+0.1% isopropylamine): RT 21.8 minutes

¹H NMR (400 MHZ, CDCl₃) δ 7.21-7.14 (m, 1H), 7.09 (d, J=7.5 Hz, 1H), 7.01 (d, J=8.1 Hz, 1H), 6.91-6.85 (m, 1H), 6.22 (s, 1H), 5.09-4.97 (m, 1H), 4.35 (br d, J=9.4 Hz, 1H), 4.32 (s, 1H), 4.29-4.12 (m, 2H), 4.03 (br t, J=10.0 Hz, 1H), 3.83 (br s, 1H), 3.80-3.74 (m, 1H), 3.56 (d, J=11.6 Hz, 1H), 3.55-3.46 (m, 1H), 3.37-3.31 (m, 1H), 3.24 (dd, J=13.6, 5.3 Hz, 1H), 2.57-2.41 (m, 2H), 2.34 (d, J=13.6 Hz, 1H), 2.15-2.00 (m, 1H), 1.44 (d, J=6.4 Hz, 3H), 2.25-1.15 (m, 8H).

### Example 4d: Peak 4 (Stereochemistry Arbitrarily Assigned at Pyrrolidine and Methyl Center))

LCMS (Method C): [M+H]⁺ m/z 429.3, RT 0.97 minutes.

Chiral analysis (Chiralpak AD-H, 25×0.46 cm, 5 μm, 50:50 n-Hexane: (ethanol+0.1% isopropylamine): RT 27.4 minutes

¹H NMR (400 MHZ, CDCl₃) δ 7.16-7.05 (m, 2H), 6.96-6.86 (m, 2H), 6.47-6.36 (m, 1H), 4.75-4.63 (m, 1H), 4.49 (dd, J=9.6, 3.1 Hz, 1H), 4.29-4.15 (m, 3H), 4.09 (td, J=9.8, 3.9 Hz, 1H), 3.82 (br s, 1H), 3.75 (d, J=11.6 Hz, 1H), 3.61 (d, J=11.6 Hz, 1H), 3.58-3.48 (m, 1H), 3.28 (d, J=9.6 Hz, 1H), 2.98 (dd, J=14.5, 9.9 Hz, 1H), 2.54-2.41 (m, 2H), 2.40-2.27 (m, 2H), 2.24-2.16 (m, 1H), 2.09 (td, J=8.3, 3.7 Hz, 1H), 2.08-1.96 (m, 1H), 1.82 (br d, J=13.6 Hz, 1H), 1.59-1.47 (m, 1H), 1.45 (d, J=5.9 Hz, 3H), 1.50-1.39 (m, 2H), 1.42-1.23 (m, 1H).

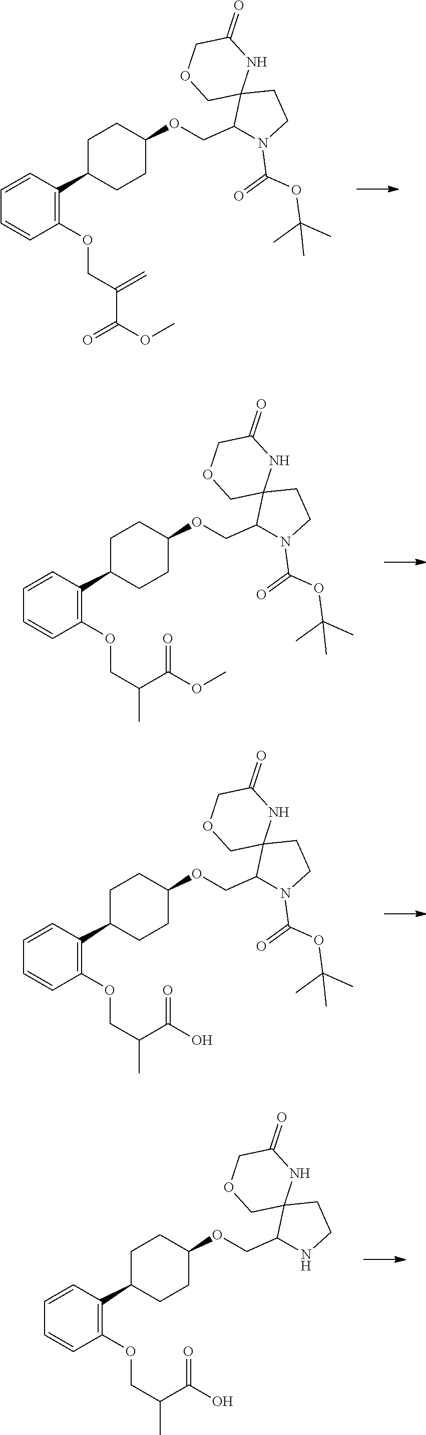 [SMILES: C=C(COc1ccccc1[C@H]1CC[C@@H](OCC2N(C(=O)OC(C)(C)C)CCC23COCC(=O)N3)CC1)C(=O)OC ; COC(=O)C(C)COc1ccccc1[C@H]1CC[C@@H](OCC2N(C(=O)OC(C)(C)C)CCC23COCC(=O)N3)CC1 ; CC(COc1ccccc1[C@H]1CC[C@@H](OCC2N(C(=O)OC(C)(C)C)CCC23COCC(=O)N3)CC1)C(=O)O ; CC(COc1ccccc1[C@H]1CC[C@@H](OCC2NCCC23COCC(=O)N3)CC1)C(=O)O]

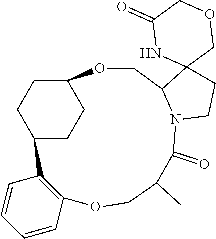 [SMILES: CC1COc2ccccc2[C@H]2CC[C@H](CC2)OCC2N(CCC23COCC(=O)N3)C1=O]

### Intermediate 20

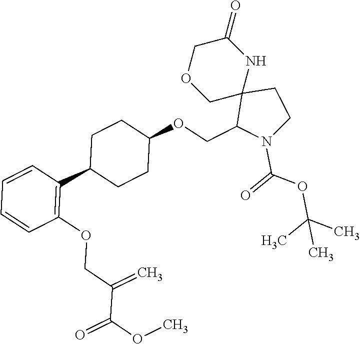 [SMILES: C=C(COc1ccccc1[C@H]1CC[C@@H](OCC2N(C(=O)OC(C)(C)C)CCC23COCC(=O)N3)CC1)C(=O)OC]

### tert-butyl-rel-(1R,5S)-7-oxo-1-({[(1s,4s)-4-[2-(3-methoxy-2-methylidene-3-oxopropoxy)phenyl]cyclohexyl]oxy}methyl)-9-oxa-2,6-diazaspiro[4.5]decane-2-carboxylate

To a solution of Intermediate 8 (200 mg, 0.434 mmol) and methyl 2-(bromomethyl) prop-2-enoate (98%, 0.080 mL, 0.651 mmol) in THF (2.5 mL) was added dipotassium carbonate (181 mg, 1.31 mmol) and the solution was stirred at room temperature overnight. The reaction was heated to 60° C. and stirred at that temperature for 5 h. The reaction was cooled to room temperature, the solids were filtered out and the filtrate concentrated in vacuo suspended in water (5 mL) and extracted with DCM (3×5 mL). The solvent was removed in vacuo to afford the crude material. The crude material was purified by silica gel column chromatography (0-100% EtOAc in heptane), to afford the title compound (200 mg) as a colorless oil. [M+H]⁺ m/z=559.4

### Intermediate 21

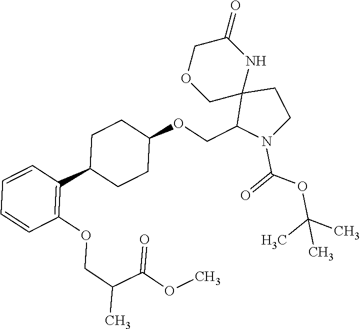 [SMILES: COC(=O)C(C)COc1ccccc1[C@H]1CC[C@@H](OCC2N(C(=O)OC(C)(C)C)CCC23COCC(=O)N3)CC1]

### tert-butyl-rel-(1R,5S)-7-oxo-1-({[(1s,4s)-4-[2-(3-methoxy-2-methyl-3-oxopropoxy)phenyl]cyclohexyl]oxy}methyl)-9-oxa-2,6-diazaspiro[4.5]decane-2-carboxylate

Intermediate 20 (200 mg, 0.358 mmol) was dissolved in ethanol (15 mL) and the atmosphere was evacuated and backfilled with nitrogen 3 times. Palladium on carbon (10%, 38 mg, 0.0358 mmol) was added and the atmosphere was evacuated and backfilled with hydrogen three times. The reaction was stirred for 5 h and then filtered through celite, washing with EtOAc, and concentrated in vacuo to afford the title compound (200 mg) as a colorless oil. [M+H]⁺ m/z=561.4

### Intermediate 22

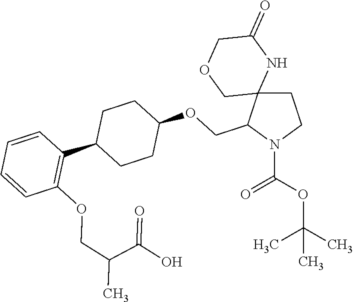 [SMILES: CC(COc1ccccc1[C@H]1CC[C@@H](OCC2N(C(=O)OC(C)(C)C)CCC23COCC(=O)N3)CC1)C(=O)O]

### 2-methyl-3-{2-[(1s,4s)-4-{[rel-(1R,5S)-2-[(tert-butoxy)carbonyl]-7-oxo-9-oxa-2,6-diazaspiro[4.5]decan-1-yl]methoxy}cyclohexyl]phenoxy}propanoic acid

To a solution of Intermediate 21 (200 mg, 0.357 mmol) in THF (10 mL) and Water (3 mL), aqueous 2 M lithium hydroxide (8.9 mL, 17.8 mmol) was added at room temperature. The mixture was stirred at room temperature for 18 hours. The mixture was diluted with water (5 mL) and neutralized to pH 7 with HCL (1M) and the aqueous phase extracted with 10% MeOH in DCM (3×10 mL). The organic layer was dried (MgSO₄) and concentrated in vacuo to afford the title compound (200 mg) as a colorless oil. [M+H]⁺ m/z=547.4

### Intermediate 23

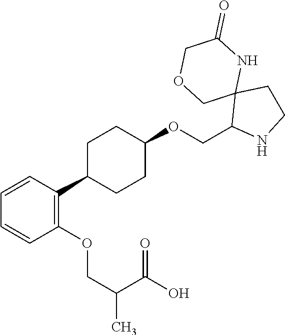 [SMILES: CC(COc1ccccc1[C@H]1CC[C@@H](OCC2NCCC23COCC(=O)N3)CC1)C(=O)O]

### 2-methyl-3-{2-[(1s,4s)-4-{[rel-(1R,5S)-7-oxo-9-oxa-2,6-diazaspiro[4.5]decan-1-yl]methoxy}cyclohexyl]phenoxy}propanoic acid

4 M hydrogen chloride in dioxane (0.37 mL, 1.46 mmol) was added to Intermediate 22 (200 mg, 0.366 mmol) at room temperature and the reaction was stirred for 30 min. The aqueous phase was neutralized with a few drops K₂CO₃ (1M) washed with DCM (2×2 mL) and concentrated in vacuo to afford the title compound (200 mg) as a beige solid. [M+H]⁺ m/z=447.3

### Example 5

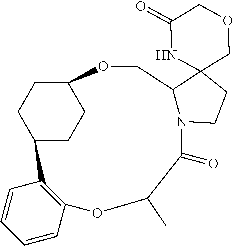 [SMILES: CC1Oc2ccccc2[C@H]2CC[C@H](CC2)OCC2N(CCC23COCC(=O)N3)C1=O]

### (1′s,3S,16′R,19′s)-10′-methyl-8′,18′-dioxa-12′-azaspiro[morpholine-3,15-′tetracyclo[17.2.2.0^{2,7}0.0^{12,16}]tricosane]-2′(7′), 3′,5′-triene-5, 11′-dione

To a stirred solution of HATU (255 mg, 0.671 mmol) and DIPEA (232 μL, 1.33 mmol) in acetonitrile (112 mL) was added Intermediate 22 (200 mg, 0.448 mmol) in acetonitrile (9.8 mL) over 2h using a syringe pump. The resulting solution was stirred for one hours. The reaction mixture was concentrated in vacuo to afford the crude material. The mixture was diluted in water and extracted with DCM (3×5 mL). The combined organic extracts were dried (MgSO₄), filtered and concentrated to afford solid residue. The crude material was purified by basic reverse phase column chromatography (10-50% acetonitrile in water (0.1% ammonia), to afford the title compound (18 mg) as a white solid.

LCMS (Method A): [M+H]⁺ m/z 429.3, RT 3.12 and 3.17 minutes

¹H NMR (500 MHZ, CDCl₃) δ 7.17-6.76 (m, 4H), 6.18 (s, 1H), 4.72-4.42 (m, 1.5H), 4.35-4.14 (m, 3H), 4.03-3.90 (m, 1H), 3.87-3.80 (m, 1.5H), 3.75 (t, J=11.5 Hz, 1H), 3.70-3.59 (m, 1H), 3.60-3.40 (m, 3H), 3.31-3.19 (m, 1H), 2.59-2.25 (m, 3H), 2.22-1.74 (m, 4H), 1.50-1.24 (m, 4H), 1.16 (dd, J=20.8, 7.1 Hz, 3H).

### Examples 5a, 5b and 5c

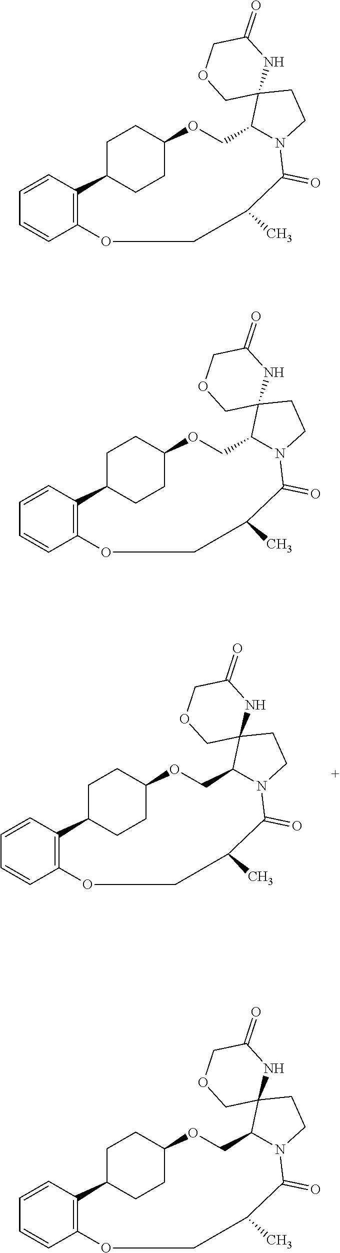 [SMILES: C[C@@H]1COc2ccccc2[C@H]2CC[C@H](CC2)OC[C@H]2N(CC[C@]23COCC(=O)N3)C1=O ; C[C@H]1COc2ccccc2[C@H]2CC[C@H](CC2)OC[C@H]2N(CC[C@]23COCC(=O)N3)C1=O ; C[C@H]1COc2ccccc2[C@H]2CC[C@H](CC2)OC[C@@H]2N(CC[C@@]23COCC(=O)N3)C1=O ; C[C@@H]1COc2ccccc2[C@H]2CC[C@H](CC2)OC[C@@H]2N(CC[C@@]23COCC(=O)N3)C1=O]

### Example 5a: (1′s,3S, 10′R,16′R,19′s)-10′-methyl-8′,18′-dioxa-12′-azaspiro[morpholine-3,15′-tetracyclo[17.2.2.0^{2,7}0.0^{12,16}]tricosane]-2′(7′),3′,5′-triene-5,11′-dione

### Example 5b: (1′s,3R,10′S,16′S,19′s)-10′-methyl-8′,18′-dioxa-12′-azaspiro[morpholine-3,15′-tetracyclo[17.2.2.0^{2,7}0.0^{12,16}]tricosane]-2′(7′),3′,5′-triene-5,11′-dione

### Example 5c: (1′s,3R,10′R,16′S,19′s)-10′-methyl-8′,18′-dioxa-12′-azaspiro[morpholine-3,15′-tetracyclo[17.2.2.0^{2,7}0.0^{12,16}]tricosane]-2′(7′),3′,5′-triene-5,11′-dione and (1′s,3S, 10′S,16′R,19′s)-10′-methyl-8′,18′-dioxa-12′-azaspiro[morpholine-3,15′-tetracyclo[17.2.2.0^{2,7}0.0^{12,16}]tricosane]-2′(7′), 3′,5′-triene-5,11′-dione

Example 5 (16 mg) was subjected to chiral preparative purification using Waters 600 eluting with 55/45% v/v n-Hexane/(ethanol+0.1% isopropylamine), Chiralpak IC (25×2.0 cm), 5 μm, flow rate 17 mL/minutes to afford the title compounds (Peak 1, 3.8 mg, 100% ee; Peak 2, 3.45 mg, 100% ee; Peak 3, 8 mg).

Example 5a Peak 1 (stereochemistry are arbitrarily assigned); ¹H NMR (500 MHz, CDCl₃) δ 7.17-7.10 (m, 1H), 7.03 (dd, J=7.3, 1.2 Hz, 1H), 6.86-6.76 (m, 3H), 4.67 (dd, J=10.2, 7.8 Hz, 1H), 4.47 (dd, J=9.7, 2.0 Hz, 1H), 4.35-4.28 (m, 1H), 4.21-4.14 (m, 1H), 3.90-3.82 (m, 1H), 3.81 (dd, J=9.1, 2.1 Hz, 1H), 3.74 (d, J=11.9 Hz, 1H), 3.68 (br s, 1H), 3.62 (dd, J=7.6, 2.8 Hz, 1H), 3.60-3.53 (m, 1H), 3.52-3.49 (m, 1H), 3.48-3.43 (m, 1H), 3.43-3.38 (m, 1H), 2.61-2.44 (m, 1H), 2.11-1.94 (m, 2H), 2.70-1.21 (m, 8H), 1.15 (d, J=7.3 Hz, 3H).

LCMS (Method C): [M+H]⁺ m/z 429.2, RT 0.98 minutes.

Chiral analysis (Chiralpak IC, 25×0.46 cm, 5 μm, 55:45 n-Hexane: (ethanol+0.1% isopropylamine)): RT 6.3 minutes

Example 5b Peak 2 (stereochemistry are arbitrarily assigned); ¹H NMR (500 MHZ, CDCl₃) δ 7.17-7.10 (m, 1H), 7.03 (dd, J=7.3, 1.2 Hz, 1H), 6.86-6.76 (m, 3H), 4.67 (dd, J=10.2, 7.8 Hz, 1H), 4.47 (dd, J=9.7, 2.0 Hz, 1H), 4.35-4.28 (m, 1H), 4.21-4.14 (m, 1H), 3.90-3.82 (m, 1H), 3.81 (dd, J=9.1, 2.1 Hz, 1H), 3.74 (d, J=11.9 Hz, 1H), 3.68 (br s, 1H), 3.62 (dd, J=7.6, 2.8 Hz, 1H), 3.60-3.53 (m, 1H), 3.52-3.49 (m, 1H), 3.48-3.43 (m, 1H), 3.43-3.38 (m, 1H), 2.61-2.44 (m, 1H), 2.11-1.94 (m, 2H), 2.70-1.21 (m, 8H), 1.15 (d, J=7.3 Hz, 3H).

LCMS (Method C): [M+H]⁺ m/z 429.2, RT 0.98 minutes.

Chiral analysis (Chiralpak IC, 25×0.46 cm, 5 μm, 55:45 n-Hexane: (ethanol+0.1% isopropylamine)): RT 8.8 minutes

Example 5c Peak 3/Peak4 (stereochemistry are arbitrarily assigned); ¹H NMR (500 MHZ, CDCl₃) δ 7.18-7.13 (m, 1H), 7.10-7.05 (m, 1H), 6.97 (d, J=8.1 Hz, 1H), 6.87 (t, J=7.3 Hz, 1H), 6.27 (s, 1H), 4.48 (dd, J=9.5, 2.8 Hz, 1H), 4.29-4.22 (m, 2H), 4.21-4.13 (m, 2H), 4.04-3.92 (m, 2H), 3.88-3.81 (m, 1H), 3.77 (d, J=11.7 Hz, 1H), 3.59 (d, J=11.7 Hz, 1H), 3.57-3.51 (m, 1H), 3.30 (d, J=9.5 Hz, 1H), 3.23 (ddd, J=10.8, 6.9, 4.2 Hz, 1H), 2.52-2.28 (m, 2H), 2.24-2.08 (m, 1H), 2.57-1.27 (m, 8H), 1.19 (d, J=7.0 Hz, 3H).

LCMS (Method C): [M+H]⁺ m/z 429.2, RT 0.99 minutes.

Chiral analysis (Chiralpak IC, 25×0.46 cm, 5 μm, 55:45 n-Hexane: (ethanol+0.1% isopropylamine)): RT 13.3, 13.8 minutes

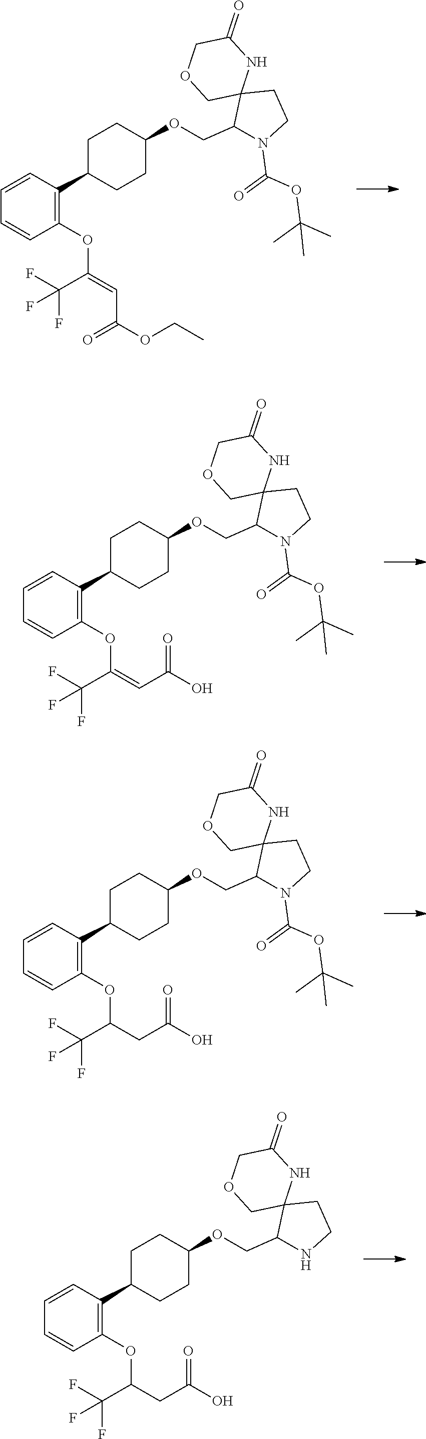 [SMILES: CCOC(=O)/C=C(/Oc1ccccc1[C@H]1CC[C@@H](OCC2N(C(=O)OC(C)(C)C)CCC23COCC(=O)N3)CC1)C(F)(F)F ; CC(C)(C)OC(=O)N1CCC2(COCC(=O)N2)C1CO[C@H]1CC[C@@H](c2ccccc2O/C(=C\C(=O)O)C(F)(F)F)CC1 ; CC(C)(C)OC(=O)N1CCC2(COCC(=O)N2)C1CO[C@H]1CC[C@@H](c2ccccc2OC(CC(=O)O)C(F)(F)F)CC1 ; O=C(O)CC(Oc1ccccc1[C@H]1CC[C@@H](OCC2NCCC23COCC(=O)N3)CC1)C(F)(F)F]

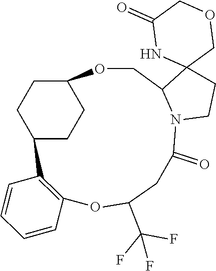 [SMILES: O=C1COCC2(CCN3C(=O)CC(C(F)(F)F)Oc4ccccc4[C@H]4CC[C@H](CC4)OCC32)N1]

### Intermediate 24

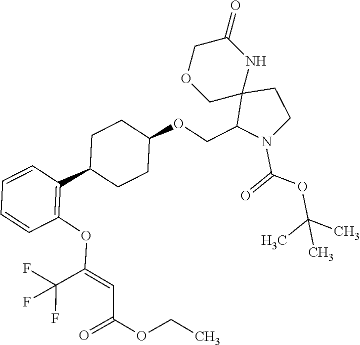 [SMILES: CCOC(=O)/C=C(/Oc1ccccc1[C@H]1CC[C@@H](OCC2N(C(=O)OC(C)(C)C)CCC23COCC(=O)N3)CC1)C(F)(F)F]

### tert-butyl-rel-(1R,5S)-7-oxo-1-({[(1s,4s)-4-(2-{[(2E)-4-ethoxy-1,1,1-trifluoro-4-oxobut-2-en-2-yl]oxy}phenyl)cyclohexyl]oxy}methyl)-9-oxa-2,6-diazaspiro[4.5]decane-2-carboxylate

To a solution of Intermediate 8 (78%, 125 mg, 0.212 mmol) in acetonitrile (1.74 mL) at room temperature under nitrogen was added 1,4-diazabicyclo[2.2.2]octane (24 μL, 0.214 mmol) and ethyl 4,4,4-trifluorobut-2-ynoate (36 μL, 0.253 mmol) and the solution stirred at room temperature for 30 min. The reaction mixture was quenched with water (5 mL) and extracted with EtOAc (2×5 mL). The organic phase was washed with brine, dried over MgSO₄, and concentrated in vacuo to afford the title compound (140 mg) as a yellow oil. [M+H]⁺ m/z 627.5

### Intermediate 25

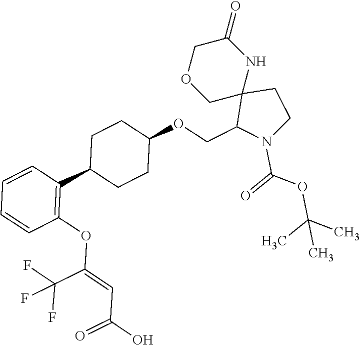 [SMILES: CC(C)(C)OC(=O)N1CCC2(COCC(=O)N2)C1CO[C@H]1CC[C@@H](c2ccccc2O/C(=C/C(=O)O)C(F)(F)F)CC1]

### 4,4,4-trifluoro-3-{2-[(1s,4s)-4-{[rel-(1R,5S)-2-[(tert-butoxy)carbonyl]-7-oxo-9-oxa-2,6-diazaspiro[4.5]decan-1-yl]methoxy}cyclohexyl]phenoxy}but-2-enoic acid

To a solution of Intermediate 24 (82%, 140 mg, 0.183 mmol) in THF (5.2 mL) and Water (1.5 mL), aqueous 2 M lithium hydroxide (4.6 mL, 9.16 mmol) was added at room temperature. The mixture was stirred at room temperature for 18 hours. The mixture was diluted with water (5 mL) neutralized to pH 7 with HCL (1M) and the aqueous phase was extracted with 10% MeOH in DCM (3×10 mL). The organic layer was dried (MgSO₄) and concentrated in vacuo to afford the title compound (130 mg) as a white solid. [M+Na]⁺ 621.2

### Intermediate 26

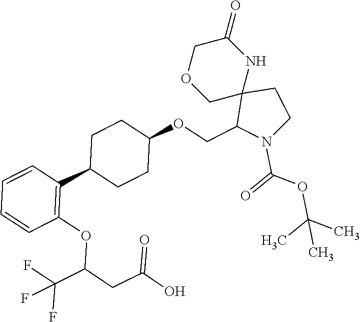 [SMILES: CC(C)(C)OC(=O)N1CCC2(COCC(=O)N2)C1CO[C@H]1CC[C@@H](c2ccccc2OC(CC(=O)O)C(F)(F)F)CC1]

### 4,4,4-trifluoro-3-{2-[(1s,4s)-4-{[rel-(1R,5S)-2-[(tert-butoxy)carbonyl]-7-oxo-9-oxa-2,6-diazaspiro[4.5]decan-1-yl]methoxy}cyclohexyl]phenoxy}butanoic acid

Intermediate 25 (78%, 130 mg, 0.169 mmol) was dissolved in ethanol (32 mL) and the atmosphere was evacuated and backfilled with nitrogen three times. Palladium on carbon (10%, 90 mg, 0.0847 mmol) was added and the atmosphere was evacuated and backfilled with hydrogen three times. The reaction was stirred for overnight and then filtered through celite, washing with EtOAc, and concentrated in vacuo to afford (100 mg) as a colorless oil. [M+H]⁺ m/z 601.35

### Intermediate 27

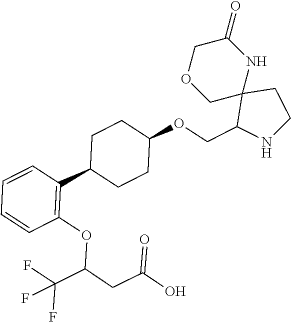 [SMILES: O=C(O)CC(Oc1ccccc1[C@H]1CC[C@@H](OCC2NCCC23COCC(=O)N3)CC1)C(F)(F)F]

### 4,4,4-trifluoro-3-{2-[(1s,4s)-4-{[rel-(1R,5S)-7-oxo-9-oxa-2,6-diazaspiro[4.5]decan-1-yl]methoxy}cyclohexyl]phenoxy}butanoic acid

4 M hydrogen chloride in dioxane (1.8 mL, 7.16 mmol) was added to Intermediate 26 (86%, 100 mg, 0.143 mmol) at room temperature and the reaction was stirred for 30 min. The reaction mixture was concentrated in vacuo to afford the title compound (97 mg) as a yellow solid. [M+H]⁺ m/z 501.3

### Example 6

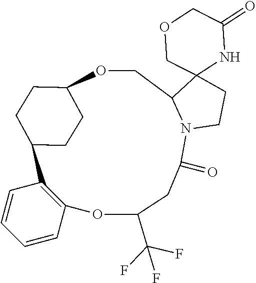 [SMILES: O=C1COCC2(CCN3C(=O)CC(C(F)(F)F)Oc4ccccc4[C@H]4CC[C@H](CC4)OCC32)N1]

### rel-(1′s,3S,16′R,19′s)-9′-(trifluoromethyl)-8′,18′-dioxa-12′-azaspiro[morpholine-3,15′-tetracyclo[17.2.2.0^{2,7}0.0^{12,16}]tricosane]-2′(7′),3′,5′-triene-5,11′-dione

To a stirred solution of HATU (82 mg, 0.216 mmol) and DIPEA (74 μL, 0.426 mmol) in acetonitrile (36 mL) was added Intermediate 28 (72 mg, 0.144 mmol) in acetonitrile (3.1 mL) over 2 hours using a syringe pump. The resulting solution was stirred for one hours. The reaction mixture was concentrated in vacuo to afford the crude material. The mixture was diluted in water and extracted with DCM (3×5 mL). The combined organic extracts were dried (MgSO₄), filtered and concentrated to give a solid residue. The crude material was purified by basic reverse phase column chromatography (10-65% acetonitrile in water (0.1% ammonia), to afford mixture of diastereoisomers (1:1) as a title compound (38 mg) as a white solid.

LCMS (Method A): [M+H]⁺ m/z 483.3, RT=3.35 and 3.39 minutes

Isomer 1: 1H NMR (500 MHZ, CDCl₃) δ 8.09 (s, 1H), 7.17-7.11 (m, 1H), 7.11-7.03 (m, 1H), 7.02-6.94 (m, 1H), 6.89-6.83 (m, 1H), 5.65-5.30 (m, 1H), 4.30-4.10 (m, 3H), 4.03-3.92 (m, 1H), 3.65-3.59 (m, 3H), 3.58-3.53 (m, 1H), 3.43-3.33 (m, 2H), 2.63 (d, J=15.7 Hz, 1H), 2.41-2.30 (m, 2H), 2.18 (d, J=14.6 Hz, 1H), 2.11-2.03 (m, 2H), 1.99-1.77 (m, 3H), 1.62-1.25 (m, 4H).

Isomer 2: 1H NMR (500 MHZ, CDCl₃) δ 7.17-7.11 (m, 2H), 7.11-7.03 (m, 1H), 7.02-6.94 (m, 1H), 6.89-6.83 (m, 1H), 5.07-4.95 (m, 1H), 4.50-4.42 (m, 2H), 4.31-4.08 (m, 3H), 3.83-3.79 (m, 2H), 3.76 (d, J=11.5 Hz, 2H), 3.70 (d, J=11.8 Hz, 1H), 3.29 (d, J=9.5 Hz, 1H), 3.21-3.11 (m, 1H), 3.05-2.89 (m, 1H), 2.83-2.74 (m, 1H), 2.58-2.49 (m, 2H), 1.98-1.75 (m, 3H), 1.65-1.24 (m, 4H).

### Example 7

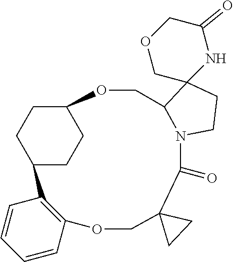 [SMILES: O=C1COCC2(CCN3C(=O)C4(CC4)COc4ccccc4[C@H]4CC[C@H](CC4)OCC32)N1]

### Rel-(1′s, 15'S,16′R,19′s)-dispiro[cyclopropane-1,10′-[8,18]dioxa-[12]azatetracyclo[17.2.2.0^{2,7}0.0^{12,16}]tricosane-15′,3″-morpholine]-2′(7′),3′,5′-triene-5″,11′-dione

Example 7 was prepared using Intermediate 8 and following the procedure described for example 2 to afford the title compound (28 mg) as a white solid.

LCMS (Method A): [M+H]⁺ m/z 441.3, RT 3.14 minutes.

¹H NMR (500 MHz, CDCl₃) δ 7.18-7.12 (m, 1H), 7.10 (dd, J=7.5, 1.5 Hz, 1H), 6.97-6.89 (m, 2H), 6.36 (s, 1H), 4.45 (dd, J=9.8, 2.1 Hz, 1H), 4.38 (td, J=10.5, 2.6 Hz, 1H), 4.33 (d, J=10.2 Hz, 1H), 4.26-4.21 (m, 2H), 4.16 (d, J=16.7 Hz, 1H), 3.81 (s, 1H), 3.72-3.64 (m, 2H), 3.62 (d, J=10.2 Hz, 1H), 3.55 (d, J=11.7 Hz, 1H), 3.31 (d, J=9.6 Hz, 1H), 2.54-2.40 (m, 2H), 2.34 (qd, J=13.1, 3.3 Hz, 1H), 2.25-2.18 (m, 1H), 2.13-2.01 (m, 2H), 1.86-1.79 (m, 1H), 1.56-1.41 (m, 3H), 1.37-1.24 (m, 1H), 1.20-1.06 (m, 3H), 0.76-0.66 (m, 1H).

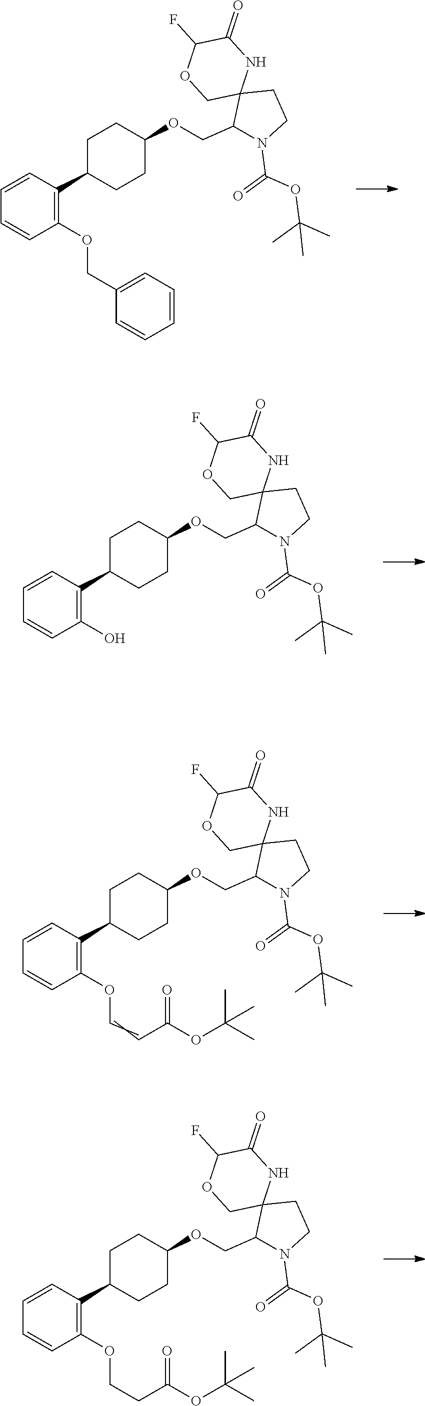 [SMILES: CC(C)(C)OC(=O)N1CCC2(COC(F)C(=O)N2)C1CO[C@H]1CC[C@@H](c2ccccc2OCc2ccccc2)CC1 ; CC(C)(C)OC(=O)N1CCC2(COC(F)C(=O)N2)C1CO[C@H]1CC[C@@H](c2ccccc2O)CC1 ; CC(C)(C)OC(=O)C=COc1ccccc1[C@H]1CC[C@@H](OCC2N(C(=O)OC(C)(C)C)CCC23COC(F)C(=O)N3)CC1 ; CC(C)(C)OC(=O)CCOc1ccccc1[C@H]1CC[C@@H](OCC2N(C(=O)OC(C)(C)C)CCC23COC(F)C(=O)N3)CC1]

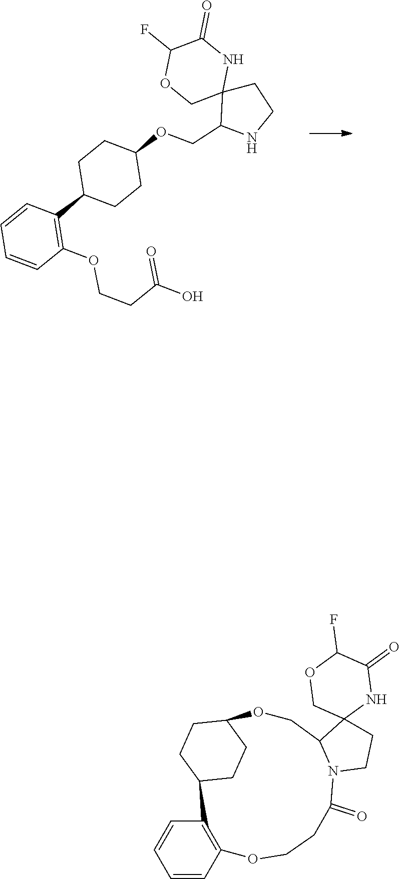 [SMILES: O=C(O)CCOc1ccccc1[C@H]1CC[C@@H](OCC2NCCC23COC(F)C(=O)N3)CC1 ; O=C1NC2(CCN3C(=O)CCOc4ccccc4[C@H]4CC[C@H](CC4)OCC32)COC1F]

### Intermediate 28

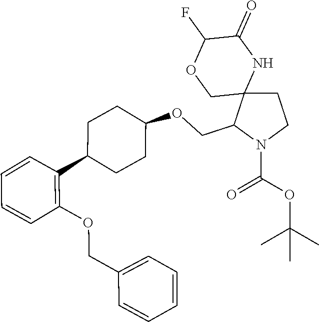 [SMILES: CC(C)(C)OC(=O)N1CCC2(COC(F)C(=O)N2)C1CO[C@H]1CC[C@@H](c2ccccc2OCc2ccccc2)CC1]

### tert-butyl-rel-(1R,5S)-8-fluoro-7-oxo-1-({[(1s,4s)-4-[2-(benzyloxy)phenyl]cyclohexyl]oxy}methyl)-9-oxa-2,6-diazaspiro[4.5]decane-2-carboxylate

A solution of 2-chloro-2-fluoro-acetyl chloride (64 mg, 0.489 mmol) in DCM (4.7 mL) was added to a stirred solution of Intermediate 6 (91%, 250 mg, 0.446 mmol) and N-ethyl-N-(propan-2-yl)propan-2-amine (0.30 mL, 1.71 mmol) in DCM (4.7 mL) at 0° C. and the mixture was stirred for 0.5 h. The reaction mixture was quenched with water (5 mL) and extracted with DCM (3×5 mL). The combined organic layers were passed through a phase separator and concentrated in vacuo to afford the crude material. The crude was purified by column chromatography (0-60% EtOAc in Heptane), to afford the corresponding amide intermediate. The residue was dissolved in THF-Anhydrous (2.6 mL) and sodium hydride (60%, 91 mg, 2.28 mmol) was slowly added at 0° C., the mixture was stirred for 30 min at this temperature and then heated at 50° C. for 2 h. The reaction mixture was quenched with water (5 ml) and extracted with DCM (2×5 ml). The combined organic extracts were dried (MgSO₄), filtered, and concentrated to afford the title compound (225 mg) as a colorless oil. [M+H]⁺ m/z 569.4

### Intermediate 29

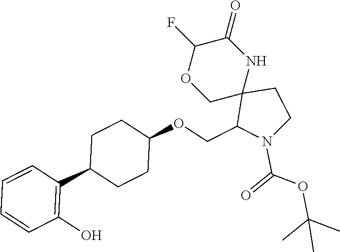 [SMILES: CC(C)(C)OC(=O)N1CCC2(COC(F)C(=O)N2)C1CO[C@H]1CC[C@@H](c2ccccc2O)CC1]

### tert-butyl-rel-(1R,5S)-8-fluoro-7-oxo-1-({[(1s,4s)-4-(2-hydroxyphenyl)cyclohexyl]oxy}methyl)-9-oxa-2,6-diazaspiro[4.5]decane-2-carboxylate

Intermediate 28 (99%, 400 mg, 0.696 mmol) was dissolved in Ethanol (33 mL) and the atmosphere was evacuated and backfilled with nitrogen three times. Palladium on carbon (10%) (5.0%, 592 mg, 0.278 mmol) was added and the atmosphere was evacuated and backfilled with hydrogen three times. The reaction was stirred for 16 hours and then filtered through celite, washing with EtOAc, and concentrated in vacuo to afford the title compound (300 mg) as a colorless oil. [M+H]⁺ m/z 479.3

### Intermediate 30

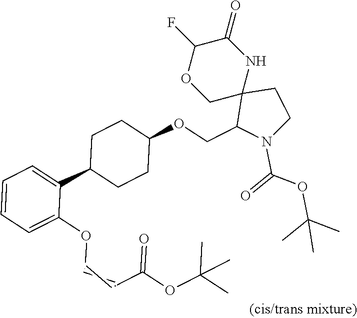

### tert-butyl-rel-(1R,5S)-8-fluoro-7-oxo-1-({[(1s,4s)-4-(2-{[3-(tert-butoxy)-3-oxoprop-1-en-1-yl]oxy}phenyl)cyclohexyl]oxy}methyl)-9-oxa-2,6-diazaspiro[4.5]decane-2-carboxylate

To a solution of tert-butyl prop-2-ynoate (86 mg, 0.680 mmol) and 1,4-diazabicyclo[2.2.2]octane (25 mg, 0.226 mmol) in THF (13 mL) at 0° C. under nitrogen was added Intermediate 29 (90%, 300 mg, 0.564 mmol) in THF (2.6 mL) and the solution stirred at room temperature for 16 hours. The reaction mixture was quenched with water (5 mL) and extracted with EtOAc (2×5 mL). The organic phase was washed with brine, dried over MgSO₄, and concentrated in vacuo to afford the crude material. The crude was purified by column chromatography (0-80% EtOAc in Heptane) to afford the title compound (215 mg) as a colorless oil. [M+H]⁺ m/z 605.5

### Intermediate 31

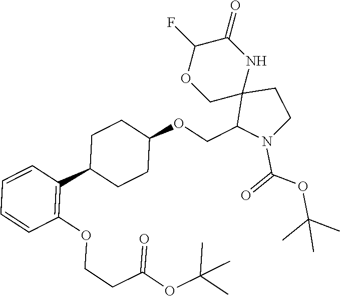 [SMILES: CC(C)(C)OC(=O)CCOc1ccccc1[C@H]1CC[C@@H](OCC2N(C(=O)OC(C)(C)C)CCC23COC(F)C(=O)N3)CC1]

### tert-butyl-rel-(1R,5S)-8-fluoro-7-oxo-1-({[(1s,4s)-4-{2-[3-(tert-butoxy)-3-oxopropoxy]phenyl}cyclohexyl]oxy}methyl)-9-oxa-2,6-diazaspiro[4.5]decane-2-carboxylate

Intermediate 30 (91%, 215 mg, 0.324 mmol) was dissolved in Ethanol (61 mL) and the atmosphere was evacuated and backfilled with nitrogen three times. Palladium on carbon (10%, 69 mg, 0.0647 mmol) was added and the atmosphere was evacuated and backfilled with hydrogen three times. The reaction was stirred for 5 hours and then filtered through celite, washed with EtOAc, and concentrated in vacuo to afford the title compound (240 mg, 0.306 mmol) as a colorless oil. [M+Na]⁺ m/z 624.6

### Intermediate 32

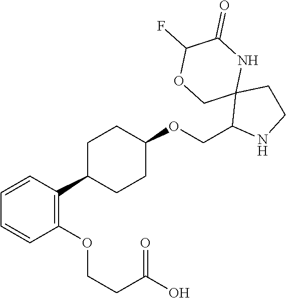 [SMILES: O=C(O)CCOc1ccccc1[C@H]1CC[C@@H](OCC2NCCC23COC(F)C(=O)N3)CC1]

### 3-{2-[(1s,4s)-4-{[rel-(1R,5S)-8-fluoro-7-oxo-9-oxa-2,6-diazaspiro[4.5]decan-1-yl]methoxy}cyclohexyl]phenoxy}propanoic acid

4 M hydrogen chloride in dioxane (3.7 mL, 14.8 mmol) was added to Intermediate 31 (99%, 180 mg, 0.294 mmol) at room temperature and the reaction was stirred for 2.5 hours. The reaction mixture was concentrated in vacuo to afford the title compound (130 mg) as a white solid. [M+H]⁺ m/z 451.3

### Example 8

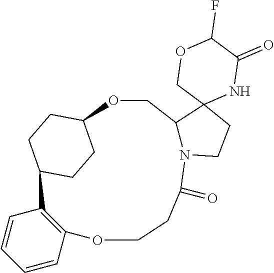 [SMILES: O=C1NC2(CCN3C(=O)CCOc4ccccc4[C@H]4CC[C@H](CC4)OCC32)COC1F]

### rel-(1′s,3S,16′R,19′s)-6-fluoro-8′,18′-dioxa-12′-azaspiro[morpholine-3,15′-tetracyclo[17.2.2.0^{2,7}0.0^{12,16}]tricosane]-2′(7′),3′,5′-triene-5,11′-dione

To a stirred solution of HATU (162 mg, 0.426 mmol) and DIPEA (147 μL, 0.843 mmol) in Acetonitrile (72 mL) was added Intermediate 32 (99%, 130 mg, 0.286 mmol) in Acetonitrile (6.2 mL) over 2h using a syringe pump. The resulting solution was stirred for one hours. The reaction mixture was concentrated in vacuo to afford the crude material. The mixture was diluted in water and extracted with DCM (3×5 mL). The combined organic extracts were dried (MgSO₄), filtered and concentrated to give a solid residue. The crude material was purified by basic reverse phase column chromatography (10-65% acetonitrile in water (0.1% ammonia), to afford the title compound (57 mg) as a yellow solid.

LCMS (Method A): [M+H]⁺ m/z 433.3, RT 3.14 and 3.22 minutes.

¹H NMR (500 MHZ, CDCl₃) δ 7.12-7.05 (m, 1H), 7.03-6.98 (m, 1H), 6.96-6.90 (m, 1H), 6.85-6.78 (m, 1H), 6.57 (s, 1H), 5.49 (dd, J=51.8, 7.1 Hz, 1H), 4.44-4.40 (m, 1H), 4.41-4.34 (m, 1H), 4.33 (dd, J=9.4, 2.9 Hz, 1H), 4.19-4.10 (m, 1H), 3.96 (d, J=11.8 Hz, 1H), 3.77-3.70 (m, 2H), 3.53-3.43 (m, 1H), 3.15 (d, J=9.5 Hz, 1H), 3.01 (dd, J=11.3, 7.0 Hz, 1H), 2.39-2.33 (m, 2H), 2.33-2.25 (m, 1H), 2.18 (td, J=12.7, 3.7 Hz, 1H), 2.12-2.05 (m, 2H), 1.97-1.90 (m, 1H), 1.78-1.70 (m, 1H), 1.52-1.44 (m, 1H), 1.44-1.37 (m, 1H), 1.29 (t, J=14.1 Hz, 3H).

### Example 9

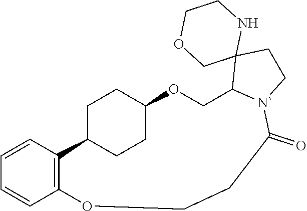

### Rel-(1′s,3S,16′R,19′s)-8′,18′-dioxa-12′-azaspiro[morpholine-3,15′-tetracyclo[17.2.2.0^{2,7}0.0^{12,16}]tricosane]-2′,4′,6′-trien-11′-one

To a stirred solution of HATU (481 mg, 1.27 mmol) and DIPEA (442 μL, 2.53 mmol) in acetonitrile (58 mL) was added Intermediate 37 (415 mg) in DMF (5.8 mL) over 2h using a syringe pump. The resulting solution was stirred for one hour. The reaction mixture was concentrated in vacuo to afford the crude material. The mixture was diluted in water and extracted with DCM (3×5 mL) The combined organic extracts were dried (MgSO₄), filtered and concentrated to give a solid residue. The crude material was purified by basic reverse phase column chromatography (20-60% acetonitrile in water (0.1% ammonia)) to afford the title compound (136 mg) as a yellow solid.

LCMS (Method A): [M+H]⁺ m/z 401.4, RT 3.17 min

¹H NMR (500 MHZ, CDCl₃) δ 7.21-7.09 (m, 1H), 7.09-7.03 (m, 1H), 7.02-6.95 (m, 1H), 6.91-6.83 (m, 1H), 4.46-4.38 (m, 1H), 4.38-4.30 (m, 1H), 4.26-4.17 (m, 2H), 4.03-3.92 (m, 1H), 3.79 (s, 1H), 3.77-3.71 (m, 2H), 3.56-3.48 (m, 2H), 3.45 (d, J=11.2 Hz, 1H), 3.32 (d, J=9.4 Hz, 1H), 3.22-3.06 (m, 1H), 3.03-2.92 (m, 2H), 2.47-2.29 (m, 3H), 2.32-2.23 (m, 1H), 2.21-2.10 (m, 3H), 1.85-1.78 (m, 2H), 1.53-1.41 (m, 2H), 1.37-1.21 (m, 2H).

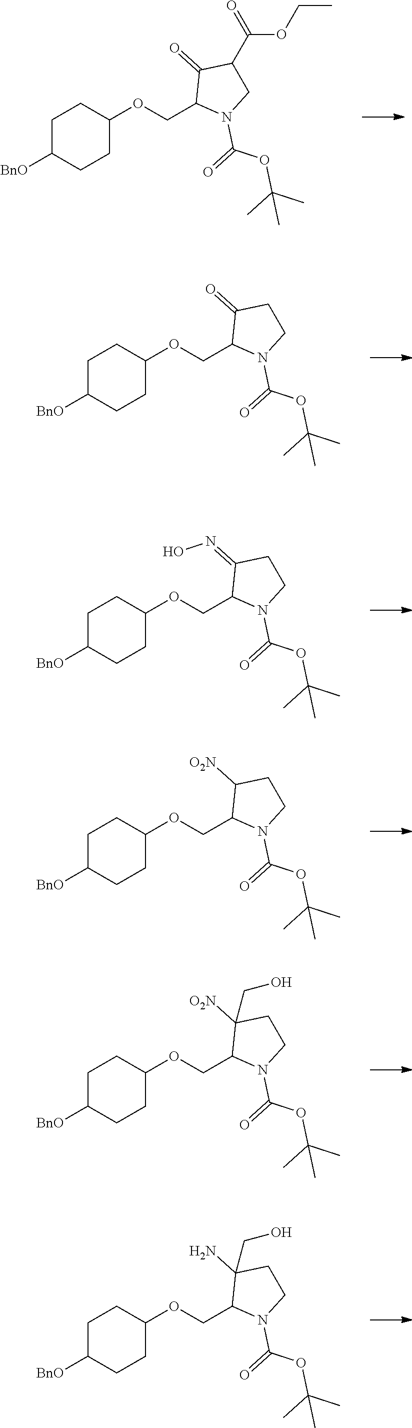 [SMILES: CCOC(=O)C1CN(C(=O)OC(C)(C)C)C(COC2CCC(OCc3ccccc3)CC2)C1=O ; CC(C)(C)OC(=O)N1CCC(=O)C1COC1CCC(OCc2ccccc2)CC1 ; CC(C)(C)OC(=O)N1CC/C(=N/O)C1COC1CCC(OCc2ccccc2)CC1 ; CC(C)(C)OC(=O)N1CCC([N+](=O)[O-])C1COC1CCC(OCc2ccccc2)CC1 ; CC(C)(C)OC(=O)N1CCC(CO)([N+](=O)[O-])C1COC1CCC(OCc2ccccc2)CC1 ; CC(C)(C)OC(=O)N1CCC(N)(CO)C1COC1CCC(OCc2ccccc2)CC1]

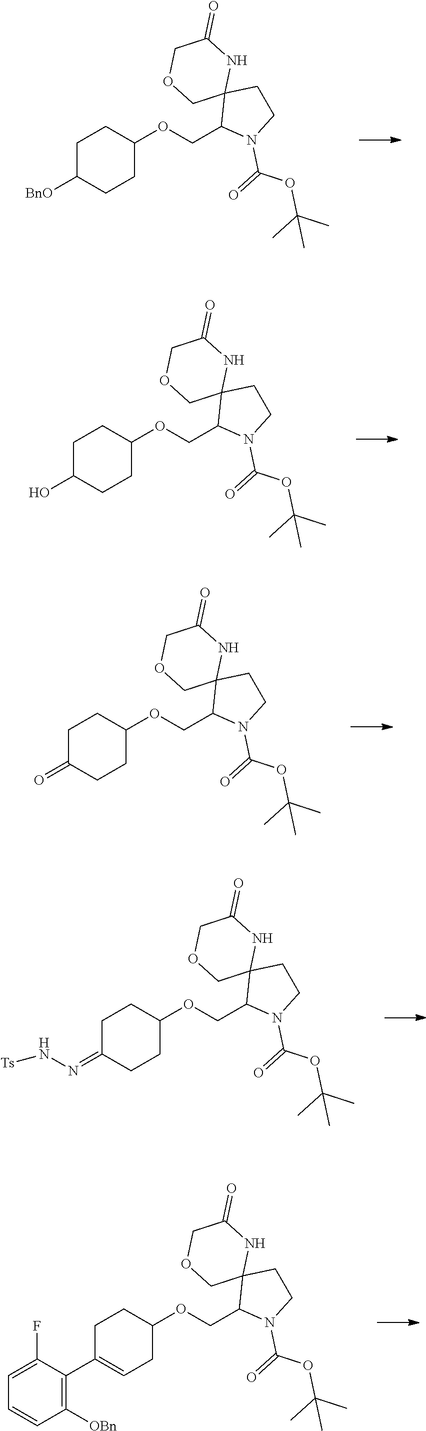 [SMILES: CC(C)(C)OC(=O)N1CCC2(COCC(=O)N2)C1COC1CCC(OCc2ccccc2)CC1 ; CC(C)(C)OC(=O)N1CCC2(COCC(=O)N2)C1COC1CCC(O)CC1 ; CC(C)(C)OC(=O)N1CCC2(COCC(=O)N2)C1COC1CCC(=O)CC1 ; Cc1ccc(S(=O)(=O)NN=C2CCC(OCC3N(C(=O)OC(C)(C)C)CCC34COCC(=O)N4)CC2)cc1 ; CC(C)(C)OC(=O)N1CCC2(COCC(=O)N2)C1COC1CC=C(c2c(F)cccc2OCc2ccccc2)CC1]

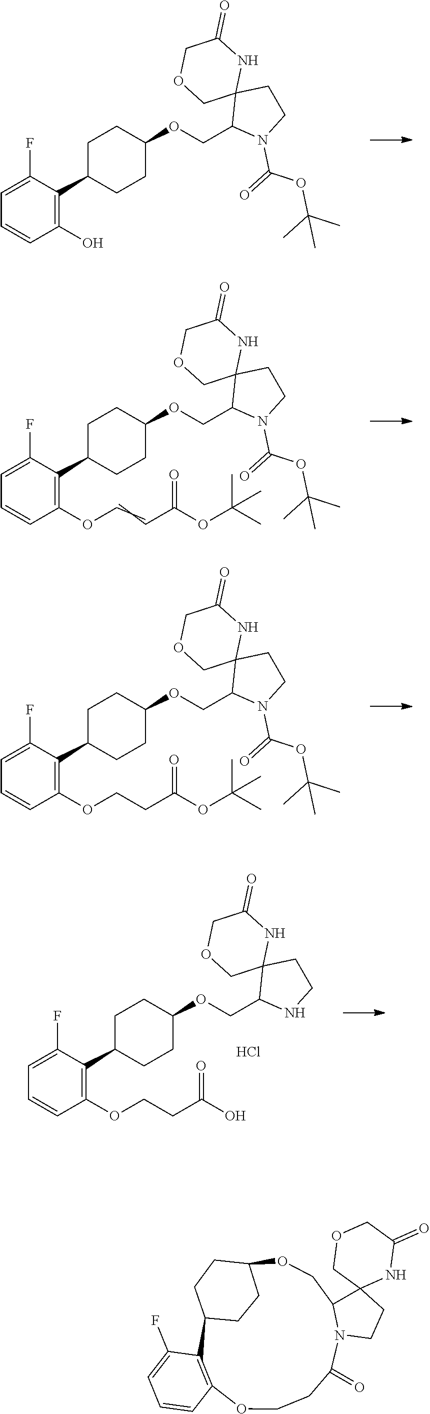 [SMILES: CC(C)(C)OC(=O)N1CCC2(COCC(=O)N2)C1CO[C@H]1CC[C@@H](c2c(O)cccc2F)CC1 ; CC(C)(C)OC(=O)C=COc1cccc(F)c1[C@H]1CC[C@@H](OCC2N(C(=O)OC(C)(C)C)CCC23COCC(=O)N3)CC1 ; CC(C)(C)OC(=O)CCOc1cccc(F)c1[C@H]1CC[C@@H](OCC2N(C(=O)OC(C)(C)C)CCC23COCC(=O)N3)CC1 ; O=C(O)CCOc1cccc(F)c1[C@H]1CC[C@@H](OCC2NCCC23COCC(=O)N3)CC1 ; Cl ; O=C1COCC2(CCN3C(=O)CCOc4cccc(F)c4[C@H]4CC[C@H](CC4)OCC32)N1]

### Intermediate 38

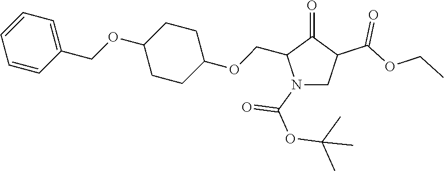 [SMILES: CCOC(=O)C1CN(C(=O)OC(C)(C)C)C(COC2CCC(OCc3ccccc3)CC2)C1=O]

### 1-tert-butyl 3-ethyl 5-({[4-(benzyloxy)cyclohexyl]oxy}methyl)-4-oxopyrrolidine-1,3-dicarboxylate

To a solution of 4-benzyloxycyclohexanol (23.50 g, 0.114 mol) in anhydrous DCM (134 mL) was added paraformaldehyde (3.40 g, 0.113 mol) followed by chloro (trimethyl) silane (20 mL, 0.155 mol). The reaction was stirred for 2.5 h at room temperature. The reaction was concentrated in vacuo at 30° C. to give a pale-yellow oil of the chloro-intermediate. In a separate flask, 2.4 M butyllithium in THF (87 mL, 0.209 mol) was added to a stirred solution of N-(propan-2-yl) propan-2-amine (29 mL, 0.207 mol) in anhydrous THF (104 mL) at −78° C. The reaction was held at this temperature for 40 min. In a third flask the freshly made LDA was added, via an addition funnel over 0.5 h, to a stirred solution of 1,3-dimethylhexahydropyrimidin-2-one (48.70 g, 0.380 mol) and 1-tert-butyl 3-ethyl 4-oxopyrrolidine-1,3-dicarboxylate (24.50 g, 95.2 mmol) in anhydrous THF (276 mL) at −78° C., the reaction temperature did not rise above −65° C. The solution was held at this temperature for 20 mins. The oil containing [4-(chloromethoxy)cyclohexyl]benzene was dissolved in anhydrous THF (67 mL) and added to the reaction mixture via an additional funnel over 20 mins, the reaction temperature did not rise above −65° C. The reaction mixture was stirred at −78° C. for 2 h. The reaction was quenched with saturated aqueous NH₄Cl. The crude mixture was diluted with water (100 mL) and extracted with EtOAc (3×300 mL). The combined organic extracts were dried over MgSO₄, filtered and concentrated in vacuo to afford the crude material. The crude material was purified by silica gel column chromatography (0-20% EtOAc in heptane) and gave the title compound (4.00 g) as a yellow oil. [M+Na]⁺: m/z 498.3

### Intermediate 39

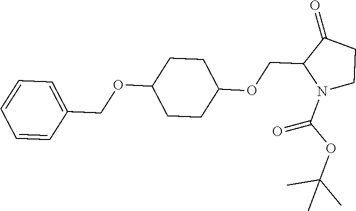 [SMILES: CC(C)(C)OC(=O)N1CCC(=O)C1COC1CCC(OCc2ccccc2)CC1]

### tert-butyl 2-({[4-(benzyloxy)cyclohexyl]oxy}methyl)-3-oxopyrrolidine-1-carboxylate

To a stirred solution of Intermediate 38 (13.50 g) in DMSO (131 mL) was added sodium chloride (3.10 g, 53.0 mmol) and water (13 mL) and the reaction mixture was heated to 125° C. for 2.5 h. The reaction mixture was cooled to room temperature, quenched with water (50 mL) and extracted with EtOAc (2×100 mL). The combined organic extracts were washed with water (2×50 mL) and brine (50 mL), dried over sodium sulfate, filtered and evaporated to dryness to afford crude material. The crude material was purified by column chromatography (0-100% EtOAc in heptane) to afford the title compound (13.5 g) as a yellow oil. [M+Na]⁺ m/z 426.3

### Intermediate 40

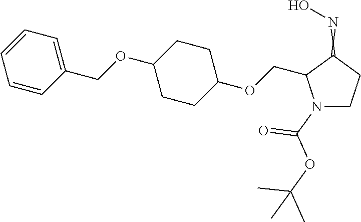 [SMILES: CC(C)(C)OC(=O)N1CCC(=NO)C1COC1CCC(OCc2ccccc2)CC1]

### tert-butyl 2-({[4-(benzyloxy)cyclohexyl]oxy}methyl)-3-(hydroxyimino) pyrrolidine-1-carboxylate

A solution of N,N-diethylethanamine (8.7 mL, 62.3 mmol), hydroxylamine hydrochloride (1:1) (4.30 g, 61.9 mmol) and Intermediate 39 (8.40 g) in ethanol (42 mL) was heated to 90° C. for 1 h. The reaction mixture was diluted with water (50 mL) and extracted with EtOAc (3×75 mL). The combined organic extracts were dried over magnesium sulfate and concentrated in vacuo to afford the title compound (8.6 g) as a yellow oil. [M+Na]⁺ m/z 441.3

### Intermediate 41

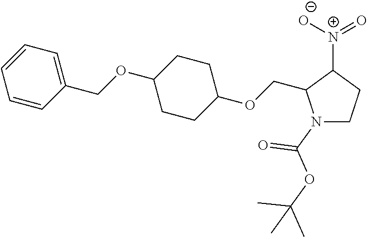 [SMILES: CC(C)(C)OC(=O)N1CCC([N+](=O)[O-])C1COC1CCC(OCc2ccccc2)CC1]

### tert-butyl 2-({[4-(benzyloxy)cyclohexyl]oxy}methyl)-3-nitropyrrolidine-1-carboxylate

A solution of trifluoroacetic anhydride (7.1 mL, 51.4 mmol) in acetonitrile (37 mL) was added to a stirred solution of hydrogen peroxide-urea (1:1) (6.8 g, 71.9 mmol) in acetonitrile (37 mL) at 0° C. and the mixture was stirred at 0° C. for 2 h. The resulting solution was added dropwise to a mixture of Intermediate 40 (8.60 g) and sodium hydrogen carbonate (8.6 g, 0.103 mol) in acetonitrile (37 mL) at 80° C. for 1 h. The reaction mixture was cooled to room temperature, quenched with sat. aq. Na₂SO₃ (50 mL) and stirred for 10 min then extracted with EtOAc (2×100 mL). The combined organic extracts were dried over MgSO₄, filtered and concentrated in vacuo to afford the crude material. The crude material was purified by silica gel column chromatography (0-70% EtOAc in heptane) to afford the title compound (4.80 g) as a pale-yellow oil. [M+H]⁺ m/z 435.3

### Intermediate 42

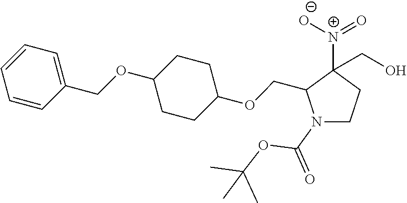 [SMILES: CC(C)(C)OC(=O)N1CCC(CO)([N+](=O)[O-])C1COC1CCC(OCc2ccccc2)CC1]

### tert-butyl-rel-(2R,3S)-2-({[4-(benzyloxy)cyclohexyl]oxy}methyl)-3-(hydroxymethyl)-3-nitropyrrolidine-1-carboxylate

Formaldehyde (37% in water, 7.4 mL, 0.10 mol) was added to a solution of Intermediate 41 (4.80 g) and triethylamine (1.9 mL, 13.3 mmol) in THF (56 mL) at room temperature. The solution was heated to 70° C. for 18 h. After cooling the reaction mixture was diluted with water (50 mL) and extracted with EtOAc (3×75 mL). The combined organic extracts were washed with brine (50 mL), dried (MgSO₄), filtered and concentrated in vacuo to afford the crude material. The crude material was purified by silica gel column chromatography (0-90% EtOAc in heptane) to afford the title compound (4.0 g) as a colorless oil. [M+H]⁺ m/z 465.5

### Intermediate 43

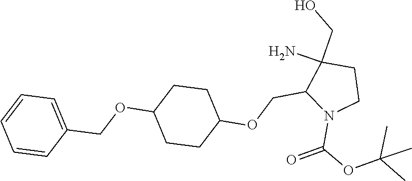 [SMILES: CC(C)(C)OC(=O)N1CCC(N)(CO)C1COC1CCC(OCc2ccccc2)CC1]

### tert-butyl-rel-(2R,3S)-3-amino-2-({[4-(benzyloxy)cyclohexyl]oxy}methyl)-3-(hydroxymethyl)pyrrolidine-1-carboxylate

A suspension of Intermediate 42 (4.00 g) and zinc (5.60 g, 85.6 mmol) in acetic acid (39 mL) and ethanol (298 mL) was stirred for 6 h at room temperature. The reaction mixture was filtered through a pad of Celite, washing with methanol. The filtrate was neutralized with sat. aq. NaHCO₃ and extracted with DCM (3×75 mL). The combined organic extracts were dried (MgSO₄) and concentrated under vacuum to afford the title compound (3.63 g) as a colorless oil. [M+H]⁺ m/z 435.3.

### Intermediate 44

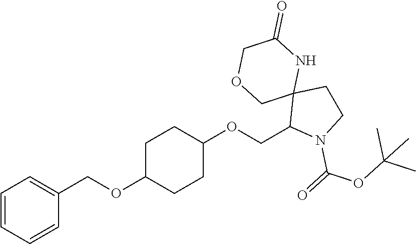 [SMILES: CC(C)(C)OC(=O)N1CCC2(COCC(=O)N2)C1COC1CCC(OCc2ccccc2)CC1]

### tert-butyl-rel-(1R,5S)-1-({[4-(benzyloxy)cyclohexyl]oxy}methyl)-7-oxo-9-oxa-2,6-diazaspiro[4.5]decane-2-carboxylate

To a solution of Intermediate 43 (3.63 g) in THF (36 mL) at 0° C. was added dipotassium carbonate (3.46 g, 25.0 mmol) then water (36 mL). To this mixture chloroacetyl chloride (0.93 mL, 11.7 mmol) was added dropwise at 0° C. The reaction was stirred at 0° C. for 1 h. The mixture was quenched with water and extracted with DCM (3×50 mL) The combined organic extracts were washed with brine (40 mL), dried (MgSO₄), filtered and concentrated to give an oily residue. This intermediate was dissolved in DCM (75 mL) and IPA (117 mL), cooled to 0° C., and potassium 2-methylpropan-2-olate (3.75 g, 33.4 mmol) was added and the reaction was stirred at 0° C. for 1 h. The reaction was quenched by addition of water (20 mL). The mixture was poured onto aqueous saturated NaHCO₃ (30 ml). After extraction with DCM (3×50 mL), the combined organic extracts were washed with brine (20 mL), dried (MgSO₄), filtered and concentrated to afford the title compound (3.72 g) as a yellow oil. [M+H]⁺ m/z 475.4.

### Intermediate 45

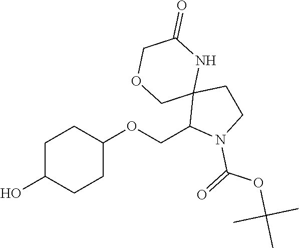 [SMILES: CC(C)(C)OC(=O)N1CCC2(COCC(=O)N2)C1COC1CCC(O)CC1]

### tert-butyl-rel-(1R,5S)-1-{[(4-hydroxycyclohexyl)oxy]methyl}-7-oxo-9-oxa-2,6-diazaspiro[4.5]decane-2-carboxylate

Intermediate 44 (2.43 g) was dissolved in Ethanol (95 mL) and the atmosphere was evacuated and backfilled with nitrogen 3 times. palladium on C (5.0%, 1013 mg, 0.476 mmol) was added and the atmosphere was evacuated and backfilled with hydrogen 3 times. The reaction was stirred for 6 hours and then filtered through Celite, washing with EtOAc and concentrated in vacuo to furnish the title compound (1.95 g) as a white solid. [M+H]⁺ m/z: 385.3

### Intermediate 46

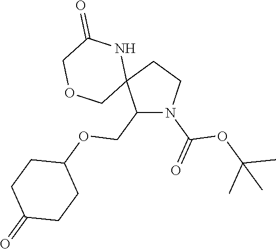 [SMILES: CC(C)(C)OC(=O)N1CCC2(COCC(=O)N2)C1COC1CCC(=O)CC1]

### tert-butyl-rel-(1R,5S)-7-oxo-1-{[(4-oxocyclohexyl)oxy]methyl}-9-oxa-2,6-diazaspiro[4.5]decane-2-carboxylate

To a solution of Intermediate 45 (1.90 g) in DCM (10 mL) at 0° C. was added Dess-Martin periodinane (2.73 g, 6.43 mmol) portion wise. The mixture was stirred at room temperature for 40 minutes, quenched by the addition of a saturated aqueous solution of Na₂S₂O₃ (10 mL) and NaHCO₃ (10 mL), and extracted with DCM (3×10 mL). The crude material was purified by silica gel column chromatography (0-100% EtOAc in heptane) to afford the title compound (1 g) as a white solid. [M+H]⁺ m/z: 383.3

### Intermediate 47

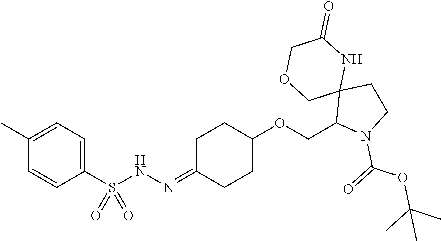 [SMILES: Cc1ccc(S(=O)(=O)NN=C2CCC(OCC3N(C(=O)OC(C)(C)C)CCC34COCC(=O)N4)CC2)cc1]

### tert-butyl-rel-(1R,5S)-1-[({4-[(4-methylbenzenesulfonamido)imino]-cyclohexyl}oxy)methyl]-7-oxo-9-oxa-2,6-diazaspiro[4.5]decane-2-carboxylate

To a stirred solution of Intermediate 46 (1.00 g) in ethanol (20 mL) was added 4-methylbenzenesulfonohydrazide (528 mg, 2.83 mmol) and the resultant solution was stirred at room temperature for 3 h. The reaction mixture was quenched with water (10 mL) and extracted with DCM (2×10 mL). The organic phase was dried (MgSO₄), filtered and concentrated in vacuo to afford the crude material. The crude material was purified by silica gel column chromatography (0-100% EtOAc in heptane) to afford the title compound (1.1 g) as a white solid. [M+H]⁺ m/z 551.4

### Intermediate 48

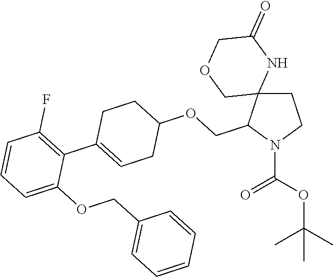 [SMILES: CC(C)(C)OC(=O)N1CCC2(COCC(=O)N2)C1COC1CC=C(c2c(F)cccc2OCc2ccccc2)CC1]

### tert-butyl-rel-(1R,5S)-1-[({4-[2-(benzyloxy)-6-fluorophenyl]cyclohex-3-en-1-yl}oxy)methyl]-7-oxo-9-oxa-2,6-diazaspiro[4.5]decane-2-carboxylate

A solution of Intermediate 47 (600 mg), (1E,4E)-1,5-diphenylpenta-1,4-dien-3-one-palladium (50 mg, 0.0545 mmol), dicyclohexyl [2′,4′,6′-tri(propan-2-yl)biphenyl-2-yl]phosphane (52 mg, 0.109 mmol), lithium tert-butoxide (218 mg, 2.72 mmol) and 1-benzyloxy-2-bromo-3-fluoro-benzene (368 mg, 1.31 mmol) in anhydrous 1,4-dioxane (10 mL) was heated to 110° C. under a nitrogen atmosphere for 18 hours. The mixture was cooled to room temperature and filtered through Celite, washed with ethyl acetate. The filtrate was concentrated in vacuo to afford the crude material. The latter was purified by column chromatography (0-100% EtOAc in heptane) to afford the title compound (452 mg) as an off white solid. [M+H]⁺ m/z 567.4

### Intermediate 49

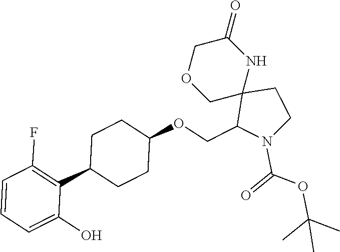 [SMILES: CC(C)(C)OC(=O)N1CCC2(COCC(=O)N2)C1CO[C@H]1CC[C@@H](c2c(O)cccc2F)CC1]

### tert-butyl-rel-(1R,5S)-7-oxo-1-({[(1s,4s)-4-(2-fluoro-6-hydroxyphenyl)-cyclohexyl]oxy}methyl)-9-oxa-2,6-

diazaspiro[4.5]decane-2-carboxylate

Intermediate 48 (450 mg) was dissolved in ethanol (20 mL) and the atmosphere was evacuated and backfilled with nitrogen 3 times. Palladium on carbon (10%, 169 mg, 0.159 mmol) was added and the atmosphere was evacuated and backfilled with hydrogen 3 times. The reaction was stirred for 18 hours and then filtered through Celite, washing with EtOAc and the filtrate was concentrated in vacuo to afford the title compound (330 mg) as an off white solid. [M+H]⁺ m/z 479.3

### Intermediate 50

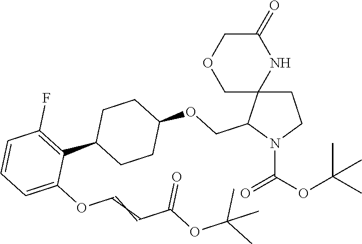 [SMILES: CC(C)(C)OC(=O)C=COc1cccc(F)c1[C@H]1CC[C@@H](OCC2N(C(=O)OC(C)(C)C)CCC23COCC(=O)N3)CC1]

### tert-butyl-rel-(1R,5S)-7-oxo-1-({[(1s,4s)-4-(2-{[(1E)-3-(tert-butoxy)-3-oxoprop-1-en-1-yl]oxy}-6-fluorophenyl)cyclohexyl]oxy}methyl)-9-oxa-2,6-diazaspiro[4.5]decane-2-carboxylate

To a solution of tert-butyl prop-2-ynoate (138 μL, 1.00 mmol) and 1,4-diazabicyclo[2.2.2]octane (16 mg, 0.143 mmol) in THF (0.5 mL) at 0° C. under nitrogen was added Intermediate 49 (330 mg) in THF (3 mL) and the solution was stirred at room temperature for 16 hours. The reaction mixture was quenched with water (10 mL) and extracted with EtOAc (2×10 mL). The combined organic phases were washed with brine, dried over MgSO₄ and concentrated in vacuo to afford the crude. The crude was purified by column chromatography (0-100% EtOAc in heptane), to afford the title compound (352 mg) as a colorless gum. [M+H]⁺ m/z 605.4.

### Intermediate 51

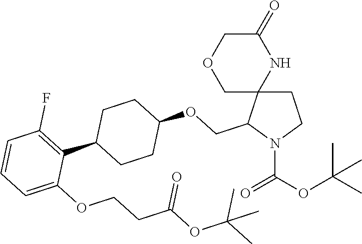 [SMILES: CC(C)(C)OC(=O)CCOc1cccc(F)c1[C@H]1CC[C@@H](OCC2N(C(=O)OC(C)(C)C)CCC23COCC(=O)N3)CC1]

### tert-butyl-rel-(1R,5S)-7-oxo-1-({[(1s,4s)-4-{2-[3-(tert-butoxy)-3-oxopropoxy]-6-fluorophenyl}cyclohexyl]oxy}methyl)-9-oxa-2,6-diazaspiro[4.5]decane-2-carboxylate

Intermediate 50 (350 mg) was dissolved in ethanol (15 mL) and the atmosphere was evacuated and backfilled with nitrogen 3 times. Palladium on carbon (10%, 123 mg, 0.12 mmol) was added and the atmosphere was evacuated and backfilled with hydrogen 3 times. The reaction was stirred for 5 hours and then filtered through Celite, washing with EtOAc and the filtrate was concentrated in vacuo to afford the title compound (303 mg) as a colorless gum. [M+H]⁺ m/z 607.4

### Intermediate 52

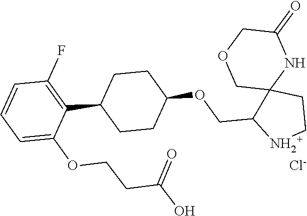 [SMILES: O=C(O)CCOc1cccc(F)c1[C@H]1CC[C@@H](OCC2NCCC23COCC(=O)N3)CC1 ; [Cl-]]

### Rel-(1R,5S)-7-oxo-1-({[(1s,4s)-4-[2-(2-carboxyethoxy)-6-fluorophenyl]cyclohexyl]oxy}methyl)-9-oxa-2,6-diazaspiro[4.5]decan-2-ium chloride

4 M Hydrogen chloride in dioxane (3.5 mL, 14.0 mmol) was added to Intermediate 51 (300 mg) at room temperature and the reaction was stirred for 2 hours. The reaction mixture was concentrated in vacuo to afford the title compound (254 mg). [M+H]⁺ m/z 451.3

### Example 10

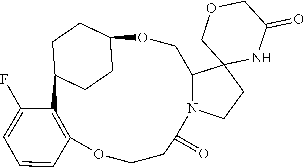 [SMILES: O=C1COCC2(CCN3C(=O)CCOc4cccc(F)c4[C@H]4CC[C@H](CC4)OCC32)N1]

### Rel-(1′s,3S,16′R,19′s)-3′-fluoro-8′,18′-dioxa-12′-azaspiro[morpholine-3,15′-tetracyclo[17.2.2.0^{2,7}0.0^{12,16}]tricosane]-2′(7′),3′,5′-triene-5,11′-dione

To a stirred solution of HATU (281 mg, 0.739 mmol) and DIPEA (430 μL, 2.46 mmol) in acetonitrile (85 mL) was added Intermediate 52 (240 mg) in anhydrous DMF (6 mL) dropwise at room temperature under nitrogen over 2 h. The reaction mixture was stirred at room temperature for 2 hours. The reaction mixture was concentrated in vacuo to afford the crude material. The crude material was diluted in water and extracted with DCM (3×20 mL). The combined organic extracts were dried (MgSO₄), filtered and concentrated to give a solid residue. The crude material was purified by basic reverse phase column chromatography (20-50% acetonitrile in water (0.1% ammonia)) to afford the title compound (99 mg) as a white solid.

LCMS (Method B): [M+H]⁺ m/z 433.3, RT 2.80 min

¹H NMR (500 MHZ, CDCl₃) δ 7.11-7.03 (m, 1H), 6.76 (d, J=8.3 Hz, 1H), 6.71-6.65 (m, 1H), 6.29 (s, 1H), 4.44 (dd, J=9.6, 3.1 Hz, 1H), 4.42-4.37 (m, 1H), 4.29-4.14 (m, 4H), 3.95 (td, J=9.9, 3.6 Hz, 1H), 3.86-3.81 (m, 1H), 3.77 (d, J=11.7 Hz, 1H), 3.60-3.53 (m, 2H), 3.28 (dd, J=9.5, 0.9 Hz, 1H), 3.14-3.00 (m, 2H), 2.46-2.34 (m, 2H), 2.27 (qd, J=12.9, 3.7 Hz, 1H), 2.20-2.07 (m, 3H), 1.81 (dt, J=14.0, 3.1 Hz, 1H), 1.64-1.60 (m, 1H), 1.46-1.33 (m, 2H), 1.32-1.23 (m, 1H).

### Examples 10a and 10b

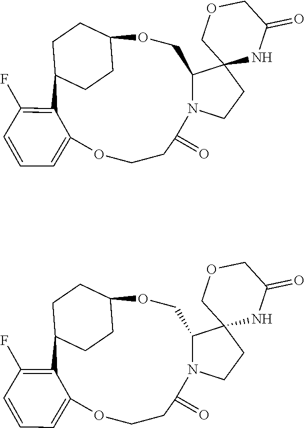 [SMILES: O=C1COC[C@@]2(CCN3C(=O)CCOc4cccc(F)c4[C@H]4CC[C@H](CC4)OC[C@H]32)N1 ; O=C1COC[C@]2(CCN3C(=O)CCOc4cccc(F)c4[C@H]4CC[C@H](CC4)OC[C@@H]32)N1]

### Example 10a: (1′s,3S,16′R,19′s)-3′-fluoro-8′,18′-dioxa-12′-azaspiro[morpholine-3,15′-tetracyclo[17.2.2.0^{2,7}0.0^{12,16}]tricosane]-2′(7′),3′,5′-triene-5,11′-dione

### Example 10b: (1′s,3R,16′S,19′s)-3′-fluoro-8′,18′-dioxa-12′-azaspiro[morpholine-3,15′-tetracyclo[17.2.2.0^{2,7}0.0^{12,16}]tricosane]-2′(7′),3′,5′-triene-5,11′-dione

Example 10 (95 mg, 0.220 mmol) was subjected to chiral preparative purification using Chiralcel OD-H, 20×250 mm, 5 μm column eluting with 85:15 Heptane: ethanol to afford the title compounds (Peak 1, 35 mg, 100% ee; and Peak 2, 30 mg, 100% ee).

### Example 10a: Peak 1 (Stereochemistry tentatively assigned at pyrrolidine)

LCMS (Method B): [M+H]⁺ m/z 433.3, RT 2.80 min

Chiral analysis (Chiralcel OD-H, 4.6×250 mm, 5 μm, 85:15 n-Hexane: Ethanol: RT 14.0 minutes

¹H NMR (500 MHz, CDCl₃) δ 7.10-7.04 (m, 1H), 6.76 (d, J=8.4 Hz, 1H), 6.71-6.65 (m, 1H), 6.30 (s, 1H), 4.44 (dd, J=9.6, 3.1 Hz, 1H), 4.42-4.37 (m, 1H), 4.30-4.15 (m, 4H), 3.95 (td, J=9.9, 3.6 Hz, 1H), 3.85-3.81 (m, 1H), 3.77 (d, J=11.6 Hz, 1H), 3.61-3.53 (m, 2H), 3.28 (dd, J=9.5, 0.9 Hz, 1H), 3.14-3.00 (m, 2H), 2.46-2.34 (m, 2H), 2.27 (qd, J=12.8, 3.7 Hz, 1H), 2.20-2.08 (m, 3H), 1.81 (dt, J=13.7, 3.1 Hz, 1H), 1.64-1.60 (m, 1H), 1.45-1.33 (m, 2H), 1.31-1.25 (m, 1H).

### Example 10b: Peak 2 (Stereochemistry Tentatively Assigned at Pyrrolidine)

LCMS (Method B): [M+H]⁺ m/z 433.3, RT 2.79 min

Chiral analysis (Chiralcel OD-H, 4.6×250 mm, 5 μm, 85:15 n-Hexane: Ethanol: RT 22.4 minutes

¹H NMR (500 MHZ, CDCl₃) δ 7.11-7.03 (m, 1H), 6.76 (d, J=8.3 Hz, 1H), 6.72-6.64 (m, 1H), 6.32 (s, 1H), 4.44 (dd, J=9.6, 3.2 Hz, 1H), 4.42-4.37 (m, 1H), 4.28-4.15 (m, 4H), 3.95 (td, J=9.9, 3.6 Hz, 1H), 3.84-3.81 (m, 1H), 3.77 (d, J=11.6 Hz, 1H), 3.61-3.53 (m, 2H), 3.28 (dd, J=9.5, 0.9 Hz, 1H), 3.13-3.00 (m, 2H), 2.46-2.34 (m, 2H), 2.27 (qd, J=12.9, 3.7 Hz, 1H), 2.20-2.06 (m, 3H), 1.81 (dt, J=13.6, 3.1 Hz, 1H), 1.59-1.54 (m, 1H), 1.44-1.33 (m, 2H), 1.31-1.26 (m, 1H).

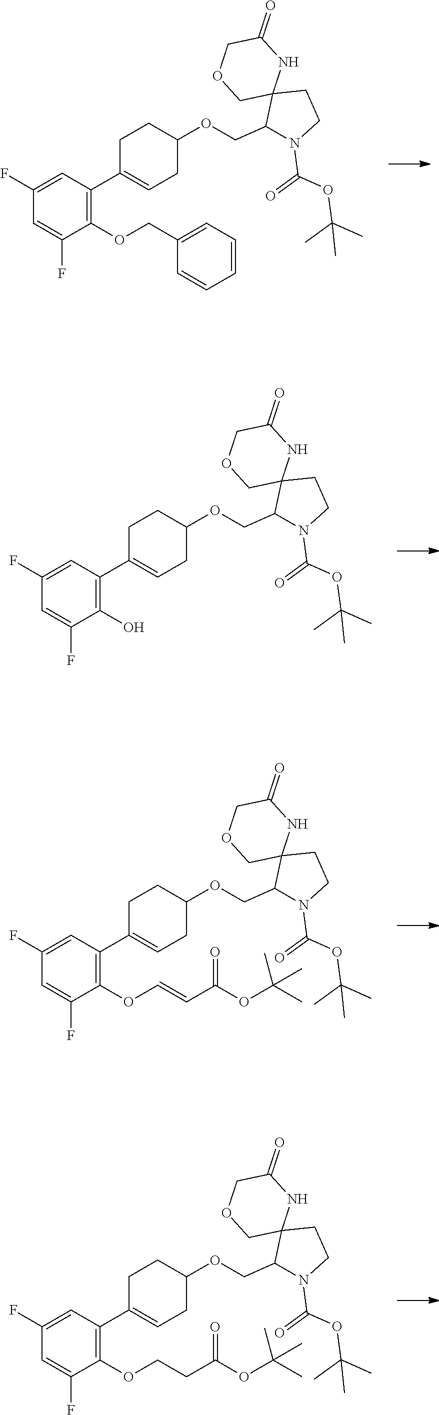 [SMILES: CC(C)(C)OC(=O)N1CCC2(COCC(=O)N2)C1COC1CC=C(c2cc(F)cc(F)c2OCc2ccccc2)CC1 ; CC(C)(C)OC(=O)N1CCC2(COCC(=O)N2)C1COC1CC=C(c2cc(F)cc(F)c2O)CC1 ; CC(C)(C)OC(=O)/C=C/Oc1c(F)cc(F)cc1C1=CCC(OCC2N(C(=O)OC(C)(C)C)CCC23COCC(=O)N3)CC1 ; CC(C)(C)OC(=O)CCOc1c(F)cc(F)cc1C1=CCC(OCC2N(C(=O)OC(C)(C)C)CCC23COCC(=O)N3)CC1]

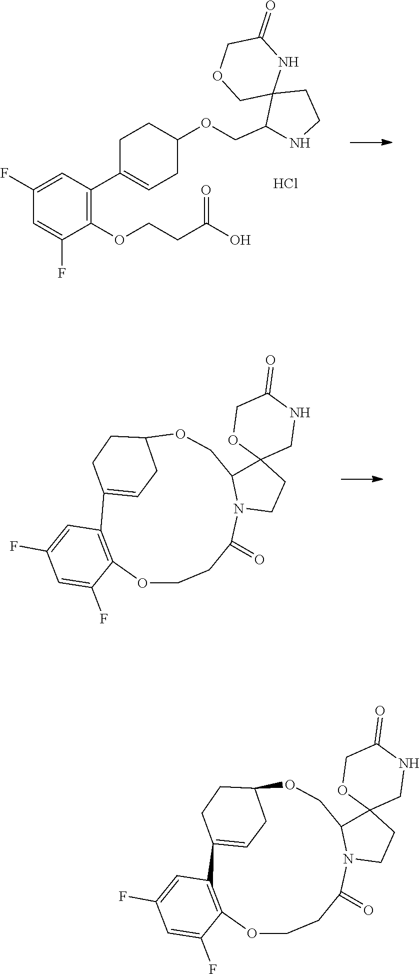

### Intermediate 53

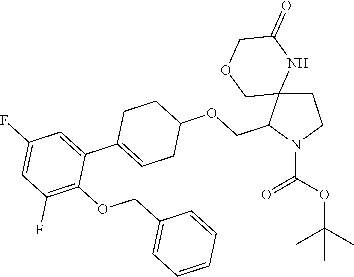 [SMILES: CC(C)(C)OC(=O)N1CCC2(COCC(=O)N2)C1COC1CC=C(c2cc(F)cc(F)c2OCc2ccccc2)CC1]

### tert-butyl-rel-(1R,5S)-1-[({4-[2-(benzyloxy)-3,5-difluorophenyl]cyclohex-3-en-1-yl}oxy)methyl]-7-oxo-9-oxa-2,6-diazaspiro[4.5]decane-2-carboxylate

A solution of Intermediate 47 (561 mg), (1 {E},4 {E})-1,5-diphenylpenta-1,4-dien-3-one:palladium (47 mg, 0.0509 mmol), dicyclohexyl-[2-(2,4,6-triisopropylphenyl)phenyl]-phosphane (49 mg, 0.102 mmol), lithium tert-butoxide (0.23 mL, 2.55 mmol) and 2-benzyloxy-1-bromo-3,5-difluoro-benzene (274 mg, 0.917 mmol) in anhydrous 1,4-dioxane (6 mL) was heated to 110° C. under a nitrogen atmosphere for 4 hours. The mixture was cooled to room temperature and filtered through Celite, washing with EtOAc (10 mL). The filtrate was concentrated in vacuo. The residue was resuspended in EtOAc (20 mL) and water (20 mL). The organic phase was separated and the aqueous phase was further extracted with EtOAc (2×20 mL). The combined organic phases were washed with brine, dried (MgSO₄) and concentrated in vacuo to afford the crude material. The crude material was purified by flash column chromatography (0-100% EtOAc in heptane) to afford the title compound (250 mg) as a yellow oil [M+H]⁺ m/z: 585.3

### Intermediate 54

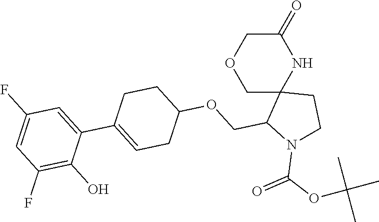 [SMILES: CC(C)(C)OC(=O)N1CCC2(COCC(=O)N2)C1COC1CC=C(c2cc(F)cc(F)c2O)CC1]

### tert-butyl-rel-(1R,5S)-1-({[(1R)-4-(3,5-difluoro-2-hydroxyphenyl)cyclohex-3-en-1-yl]oxy}methyl)-7-oxo-9-oxa-2,6-diazaspiro[4.5]decane-2-carboxylate

Intermediate 53 (300 mg) was dissolved in ethanol (10 mL) and the atmosphere was evacuated and backfilled with nitrogen 3 times. Palladium on carbon (10%, 33 mg, 0.0313 mmol) was added and the atmosphere was evacuated and backfilled with hydrogen 3 times. The reaction was stirred for 6 hours and then filtered through Celite, washing with EtOAc and the filtrate was concentrated in vacuo to afford the title compound (278 mg) as an orange oil. [M+H]⁺ m/z 495.3.

### Intermediate 55

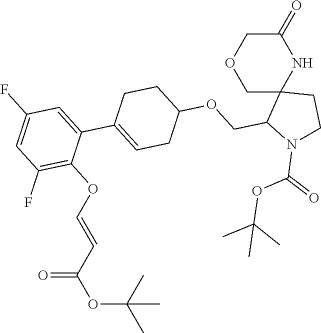 [SMILES: CC(C)(C)OC(=O)/C=C/Oc1c(F)cc(F)cc1C1=CCC(OCC2N(C(=O)OC(C)(C)C)CCC23COCC(=O)N3)CC1]

### tert-butyl-rel-(1R,5S)-1-({[(1R)-4-(2-{[(1E)-3-(tert-butoxy)-3-oxoprop-1-en-1-yl]oxy}-3,5-difluorophenyl)cyclohex-3-en-1-yl]oxy}methyl)-7-oxo-9-oxa-2,6-diazaspiro[4.5]decane-2-carboxylate

To a solution of tert-butyl prop-2-ynoate (63 mg, 0.496 mmol) and 1,4-diazabicyclo[2.2.2]octane (18 mg, 0.164 mmol) in THF (9 mL) at 0° C. under nitrogen was added Intermediate 54 (278 mg) in THF (2 mL) and the solution was stirred at room temperature for 16 hours. Further tert-butyl prop-2-ynoate (63 mg, 0.496 mmol) and 1,4-diazabicyclo[2.2.2]octane (18 mg, 0.164 mmol) in THF (2 mL) was added and the reaction mixture was stirred at room temperature for 3 hours. The reaction mixture was quenched with water (10 mL) and extracted with EtOAc (2×10 mL). The combined organic phases were washed with brine, dried over MgSO₄ and concentrated in vacuo to afford the crude material. The crude material was purified by flash column chromatography (0-100% EtOAc in heptane) to afford the title compound (206 mg) as a yellow solid [M+H]⁺: m/z 621.3

### Intermediate 56

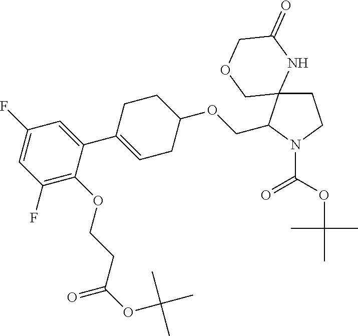 [SMILES: CC(C)(C)OC(=O)CCOc1c(F)cc(F)cc1C1=CCC(OCC2N(C(=O)OC(C)(C)C)CCC23COCC(=O)N3)CC1]

### tert-butyl-rel-(1R,5S)-1-({[(1R)-4-{2-[3-(tert-butoxy)-3-oxopropoxy]-3,5-difluorophenyl}cyclohex-3-en-1-yl]oxy}methyl)-7-oxo-9-oxa-2,6-diazaspiro[4.5]decane-2-carboxylate

Intermediate 55 (205 mg) was dissolved in ethanol (5 mL) and the atmosphere was evacuated and backfilled with nitrogen 3 times. Palladium on carbon (10%, 61 mg, 0.0576 mmol) was added and the atmosphere was evacuated and backfilled with hydrogen 3 times. The reaction was stirred for 4 hours and then filtered through Celite, washing with EtOAc and the filtrate was concentrated in vacuo to afford the title compound (195 mg) as a yellow foam [M+Na]⁺ m/z 645.3

### Intermediate 57

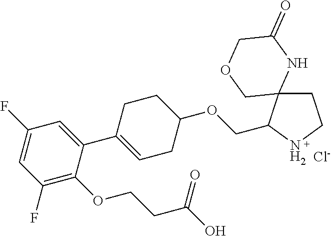 [SMILES: O=C(O)CCOc1c(F)cc(F)cc1C1=CCC(OCC2NCCC23COCC(=O)N3)CC1 ; [Cl-]]

### Rel-(1R,5S)-1-[({4-[2-(2-carboxyethoxy)-3,5-difluorophenyl]cyclohex-3-en-1-yl}oxy)methyl]-7-oxo-9-oxa-2,6-diazaspiro[4.5]decan-2-ium chloride

4 M Hydrogen chloride (2.7 mL, 11.0 mmol) was added to Intermediate 56 (136 mg) at room temperature and the reaction was stirred for 1 hour. The reaction mixture was concentrated in vacuo to afford the title compound (160 mg) as a yellow solid. [M+H]⁺: m/z 467.2

### Intermediate 58

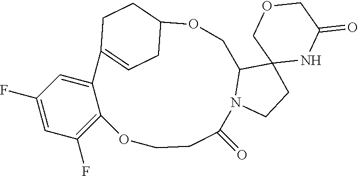 [SMILES: O=C1COCC2(CCN3C(=O)CCOc4c(F)cc(F)cc4C4=CCC(CC4)OCC32)N1]

### Rel-(3S,16′R)-4′,6′-difluoro-8′,18′-dioxa-12′-azaspiro[morpholine-3,15′-tetracyclo[17.2.2.0^{2,7}0.0^{12,16}]tricosane]-1′(21′),2′,4′,6′-tetraene-5,11′-dione

To a stirred solution of HATU (136 mg, 0.359 mmol) and DIPEA (168 μL, 0.960 mmol) in acetonitrile (60 mL) was added Intermediate 57 (112 mg) in anhydrous DMF (5 mL) over 2h using a syringe pump. The resulting solution was stirred for one hour. The reaction mixture was concentrated in vacuo to afford the crude material. The crude material was diluted in water and extracted with DCM (3×5 mL). The combined organic extracts were dried (MgSO₄), filtered and concentrated to give a solid residue. The crude material was purified by basic reverse phase column chromatography (10-100% acetonitrile in water (0.1% ammonia)) to afford the title compound (60 mg) as a yellow solid [M+H]⁺ m/z 449.5

### Example 11

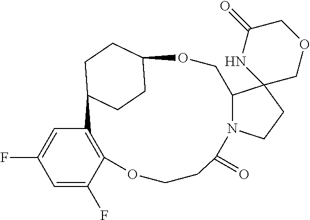 [SMILES: O=C1COCC2(CCN3C(=O)CCOc4c(F)cc(F)cc4[C@H]4CC[C@H](CC4)OCC32)N1]

### Rel-(1′s,3S,16′R,19′s)-4′,6′-difluoro-8′,18′-dioxa-12′-azaspiro[morpholine-3,15′-tetracyclo[17.2.2.0^{2,7}0.0^{12,16}]tricosane]-2′(7′),3′,5′-triene-5,11′-dione

Intermediate 58 (60 mg) was dissolved in ethanol (2.75 mL) and the atmosphere was evacuated and backfilled with nitrogen 3 times. Palladium on carbon (10%, 5.5 mg, 5.17 μmol) was added and the atmosphere was evacuated and backfilled with hydrogen 3 times. The reaction was stirred for 16 hours and then filtered through Celite, washing with EtOAc and the filtrate was concentrated in vacuo to afford the crude. The crude material was purified by basic reverse phase column chromatography (10-45% acetonitrile in water (0.1% ammonia)) to afford the title compound (3 mg) as a white solid.

LCMS (Method A): [M+H]⁺ m/z 451.3, RT 3.13 minutes

¹H NMR (500 MHZ, DMSO) δ 8.15 (s, 1H), 7.28-7.02 (m, 1H), 6.97-6.81 (m, 1H), 4.52-4.38 (m, 1H), 4.26-3.91 (m, 5H), 3.89-3.71 (m, 2H), 3.67 (s, 1H), 3.64-3.54 (m, 2H), 3.49-3.37 (m, 1H), 3.18-3.14 (m, 1H), 3.08-2.98 (m, 1H), 2.41-2.24 (m, 2H), 2.17-1.81 (m, 5H), 1.44-1.21 (m, 4H).

### Examples 12a and 12b

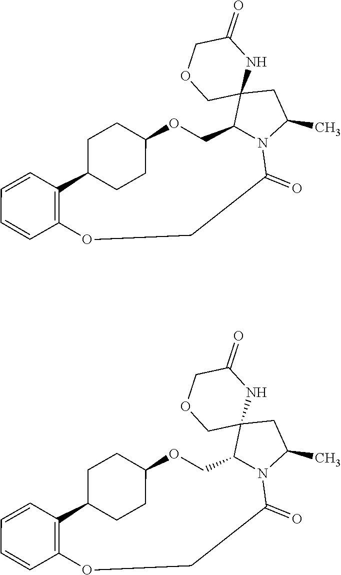 [SMILES: C[C@@H]1C[C@@]2(COCC(=O)N2)[C@@H]2CO[C@H]3CC[C@H](CC3)c3ccccc3OCC(=O)N12 ; C[C@@H]1C[C@]2(COCC(=O)N2)[C@H]2CO[C@H]3CC[C@H](CC3)c3ccccc3OCC(=O)N12]

### Example 12a: (1′s,3S,12′R,15′R,18′s)-12′-methyl-8′,17′-dioxa-11′-azaspiro[morpholine-3,14′-tetracyclo[16.2.2.0^{2,7}0.0^{11,15}]docosane]-2′(7′),3′,5′-triene-5,10′-dione

### Example 12b: (1′s,3R,12′R,15′S, 18′s)-12′-methyl-8′,17′-dioxa-11′-azaspiro[morpholine-3,14′-tetracyclo[16.2.2.0^{2,7}0.0^{11,15}]docosane]-2′(7′),3′,5′-triene-5,10′-dione

Above example 12a and 12b was prepared following the similar procedure described for example 3. The crude material was purified by acidic reverse phase column chromatography (20-40% acetonitrile in water (0.1% formic acid), to afford the title compounds example 12a (11 mg) as an orange solid and 12b (16 mg) as a yellow solid.

### Example 12a: LCMS (Method B): [M+H]⁺ m/z 415.3, RT=2.76 minutes

¹H NMR (500 MHZ, CDCl₃) δ 7.47 (s, 1H), 7.17 (t, J=7.7 Hz, 1H), 7.08 (d, J=7.3 Hz, 1H), 6.89 (t, J=7.4 Hz, 1H), 6.77 (s, 1H), 4.98 (d, J=8.4 Hz, 1H), 4.44-4.11 (m, 6H), 3.88 (s, 1H), 3.71-3.62 (m, 2H), 3.23 (d, J=9.0 Hz, 1H), 2.79 (s, 1H), 2.56 (s, 1H), 2.41-2.28 (m, 1H), 2.25-2.06 (m, 3H), 1.63 (s, 4H), 1.53-1.41 (m, 3H), 1.39-1.30 (m, 2H).

### Example 12b: LCMS (Method B): [M+H]⁺ m/z 415.3, RT=2.99 minutes

¹H NMR (500 MHZ, CDCl₃) δ 7.21-7.14 (m, 1H), 7.09 (d, J=7.4 Hz, 1H), 6.91 (t, J=7.4 Hz, 1H), 6.77 (d, J=8.0 Hz, 1H), 6.17 (s, 1H), 5.09 (d, J=10.5 Hz, 1H), 4.70 (dt, J=13.5, 6.6 Hz, 1H), 4.55 (s, 1H), 4.35-4.22 (m, 3H), 4.13 (d, J=16.7 Hz, 1H), 4.04 (d, J=11.5 Hz, 1H), 3.84 (s, 1H), 3.61 (d, J=11.6 Hz, 1H), 3.24 (d, J=9.8 Hz, 1H), 2.64 (dd, J=12.9, 9.9 Hz, 1H), 2.59-2.43 (m, 2H), 2.16-2.01 (m, 2H), 1.87-1.73 (m, 2H), 1.55-1.45 (m, 2H), 1.42-1.32 (m, 5H).

### Example 13

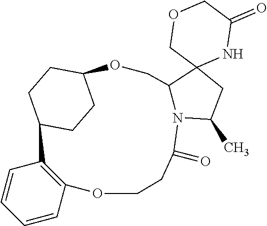 [SMILES: C[C@@H]1CC2(COCC(=O)N2)C2CO[C@H]3CC[C@H](CC3)c3ccccc3OCCC(=O)N21]

### rel-(1′s,3S, 13′R,16′R,19′s)-13′-methyl-8′,18′-dioxa-12′-azaspiro[morpholine-3,15′-tetracyclo[17.2.2.0^{2,7}0.0^{12,16}]tricosane]-2′(7′),3′,5′-triene-5,11′-dione

Example 13 was prepared using known starting materials 1-tert-butyl 3-ethyl (2R)-2-methyl-4-oxopyrrolidine-1,3-dicarboxylate and following the similar procedure described for example 1 to afford the title compound (38 mg) as an off white solid.

LCMS (Method A): [M+H]⁺ m/z 429.3, RT 3.08 and 3.17 minutes

¹H NMR (500 MHZ, CDCl₃) § 7.18-7.10 (m, 1H), 7.10-7.01 (m, 1H), 6.99-6.91 (m, 1H), 6.90-6.76 (m, 1H), 6.46-5.98 (m, 1H), 4.88-4.41 (m, 2H), 4.38-4.09 (m, 4H), 4.04-3.77 (m, 2H), 3.73-3.55 (m, 1H), 3.54-3.33 (m, 1H), 3.26-2.97 (m, 1H), 2.68-2.38 (m, 3H), 2.38-2.08 (m, 2H), 1.97-1.77 (m, 2H), 1.63-1.50 (m, 3H), 1.42-1.21 (m, 6H).

### Examples 13a and 13b

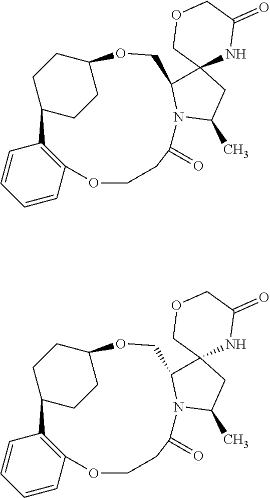 [SMILES: C[C@@H]1C[C@@]2(COCC(=O)N2)[C@@H]2CO[C@H]3CC[C@H](CC3)c3ccccc3OCCC(=O)N12 ; C[C@@H]1C[C@]2(COCC(=O)N2)[C@H]2CO[C@H]3CC[C@H](CC3)c3ccccc3OCCC(=O)N12]

### Example 13a: (1′s,3S, 13′R,16′R,19′s)-13′-methyl-8′,18′-dioxa-12′-azaspiro[morpholine-3,15′-tetracyclo[17.2.2.0^{2,7}0.0^{12,16}]tricosane]-2′(7′),3′,5′-triene-5,11′-dione

### Example 13b: (1′s,3R,13′R,16′S,19′s)-13′-methyl-8′,18′-dioxa-12′-azaspiro[morpholine-3,15′-tetracyclo[17.2.2.0^{2,7}0.0^{12,16}]tricosane]-2′(7′),3′,5′-triene-5,11′-dione

Example 13 (35 mg) was subjected to chiral preparative purification using Waters 600 eluting with 70/30% v/v n-Hexane/(ethanol+0.1% isopropylamine), Chiralpak OD-H (25×2.0 cm), 5 μm, flow rate 17 mL/minutes to afford the title compounds (Peak 1, 11.8 mg, 100% ee; and Peak 2, 6.4 mg, 100% ee).

Example 13a: Peak 1 (stereochemistry arbitrarily assigned at pyrrolidine); ¹H NMR (500 MHz, CDCl₃) δ 7.19-7.12 (m, 1H), 7.06 (ddd, J=14.5, 7.4, 1.5 Hz, 1H), 7.00-6.78 (m, 2H), 7.38-6.00 (m, 1H), 4.92-4.36 (m, 1H), 4.56-3.79 (m, 6H), 3.96-3.34 (m, 3H), 3.51-3.12 (m, 1H), 3.55-2.41 (m, 1H), 3.12-2.31 (m, 1H), 2.38-2.24 (m, 1H), 2.72-2.12 (m, 1H), 2.73-1.82 (m, 4H), 2.23-1.59 (m, 1H), 1.60-1.27 (m, 7H).

LCMS (Method C): [M+H]⁺ m/z 429.3, RT 0.97 minutes.

Chiral analysis (Chiralpak OD-H, 25×0.46 cm, 5 μm, 70:30 n-Hexane: (ethanol+0.1% isopropylamine)): RT 6.6 minutes

Example 13b: Peak 2 (stereochemistry arbitrarily assigned at pyrrolidine); ¹H NMR (500 MHz, CDCl₃) δ 7.19-7.12 (m, 1H), 7.10-7.04 (m, 1H), 6.95 (d, J=8.1 Hz, 1H), 6.86 (t, J=7.3 Hz, 1H), 6.03 (s, 1H), 4.56 (dd, J=9.6, 2.6 Hz, 1H), 4.45 (d, J=1.6 Hz, 1H), 4.41-4.29 (m, 3H), 4.29-4.20 (m, 1H), 4.18-4.08 (m, 1H), 4.02 (d, J=11.5 Hz, 1H), 3.84 (br s, 1H), 3.60 (d, J=11.5 Hz, 1H), 3.19 (d, J=9.5 Hz, 1H), 3.09 (ddd, J=15.0, 11.4, 4.0 Hz, 1H), 2.61 (dd, J=12.9, 9.7 Hz, 1H), 2.52-2.34 (m, 3H), 2.22-2.04 (m, 2H), 1.85 (d, J=13.0 Hz, 1H), 1.79 (br d, J=13.3 Hz, 1H), 1.57-1.40 (m, 2H), 1.38-1.24 (m, 5H).

LCMS (Method C): [M+H]⁺ m/z 429.2, RT 0.99 minutes.

Chiral analysis (Chiralpak OD-H, 25×0.46 cm, 5 μm, 70:30 n-Hexane: (ethanol+0.1% isopropylamine)): RT 9.5 minutes

### Example 14

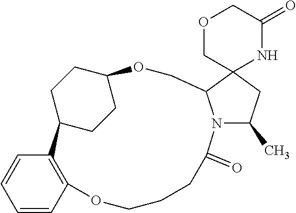 [SMILES: C[C@@H]1CC2(COCC(=O)N2)C2CO[C@H]3CC[C@H](CC3)c3ccccc3OCCCC(=O)N21]

### Example 14: (1′s,14′R,20's)-14′-methyl-8′,19′-dioxa-13′-azaspiro[morpholine-3,16′-tetracyclo[18.2.2.0^{2,7}0.0^{13,17}]tetracosane]-2′(7′),3′,5′-triene-5,12′-dione

Example 14 was prepared using known starting materials 1-tert-butyl 3-ethyl (2R)-2-methyl-4-oxopyrrolidine-1,3-dicarboxylate and following the similar procedure described for example 2 to afford the title compound (15 mg) as a yellow solid.

¹H NMR (500 MHZ, CDCl₃) δ 7.18-7.12 (m, 1H), 7.07 (dd, J=7.4, 1.9 Hz, 1H), 6.90-6.78 (m, 2H), 6.46-5.99 (m, 1H), 4.61-3.06 (m, 11H), 2.88-1.61 (m, 11H), 1.52-1.38 (m, 4H), 1.38-1.24 (m, 3H).

LCMS (Method B): [M+H]⁺ m/z 443.3, RT 3.05 minutes.

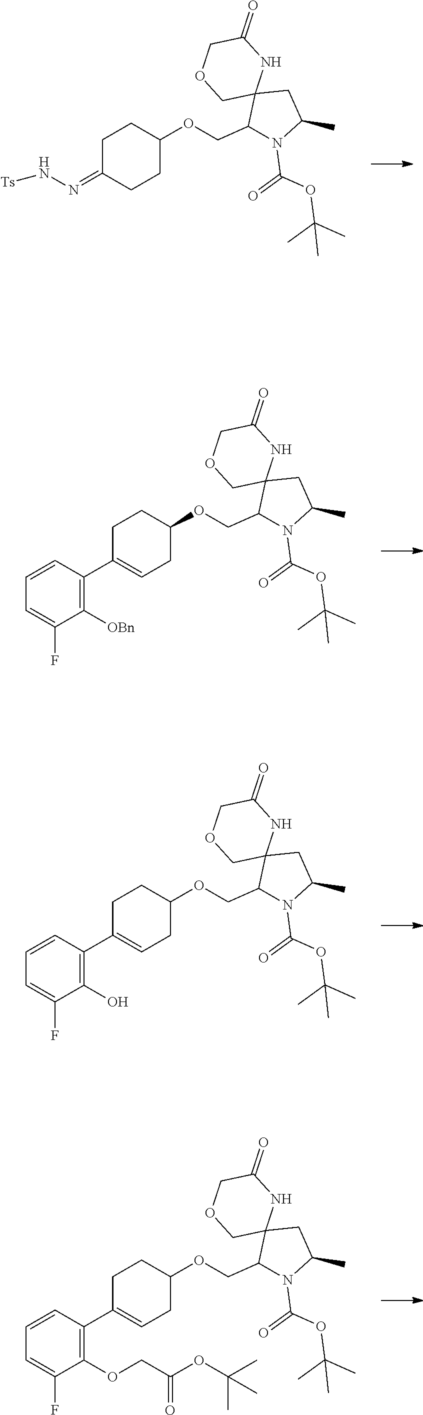 [SMILES: Cc1ccc(S(=O)(=O)NN=C2CCC(OCC3N(C(=O)OC(C)(C)C)[C@H](C)CC34COCC(=O)N4)CC2)cc1 ; C[C@@H]1CC2(COCC(=O)N2)C(CO[C@H]2CC=C(c3cccc(F)c3OCc3ccccc3)CC2)N1C(=O)OC(C)(C)C ; C[C@@H]1CC2(COCC(=O)N2)C(COC2CC=C(c3cccc(F)c3O)CC2)N1C(=O)OC(C)(C)C ; C[C@@H]1CC2(COCC(=O)N2)C(COC2CC=C(c3cccc(F)c3OCC(=O)OC(C)(C)C)CC2)N1C(=O)OC(C)(C)C]

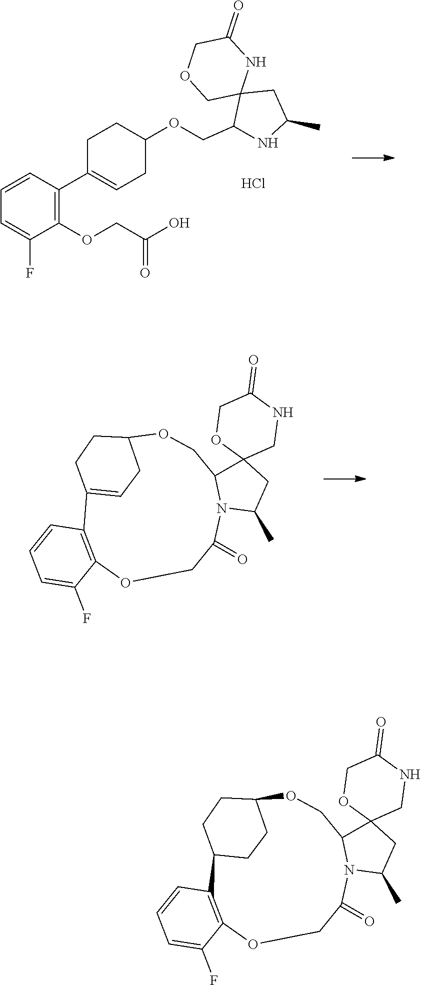 [SMILES: C[C@@H]1CC2(COCC(=O)N2)C(COC2CC=C(c3cccc(F)c3OCC(=O)O)CC2)N1 ; Cl ; C[C@@H]1CC2(CNC(=O)CO2)C2COC3CC=C(CC3)c3cccc(F)c3OCC(=O)N21 ; C[C@@H]1CC2(CNC(=O)CO2)C2CO[C@H]3CC[C@H](CC3)c3cccc(F)c3OCC(=O)N21]

### Intermediate 59

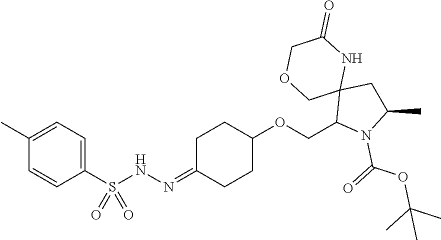 [SMILES: Cc1ccc(S(=O)(=O)NN=C2CCC(OCC3N(C(=O)OC(C)(C)C)[C@H](C)CC34COCC(=O)N4)CC2)cc1]

### tert-butyl (3R)-3-methyl-1-[({4-[(4-methylbenzenesulfonamido)imino]cyclohexyl}oxy)-methyl]-7-oxo-9-oxa-2,6-diazaspiro[4.5]decane-2-carboxylate

Intermediate 59 was prepared using the known starting material 1-tert-butyl 3-ethyl (2R)-2-methyl-4-oxopyrrolidine-1,3-dicarboxylate following a similar procedure as described for Intermediate 47 to afford the title compound (6.80 g) as a white solid. [M+H]⁺ m/z 565.3.

### Intermediate 60

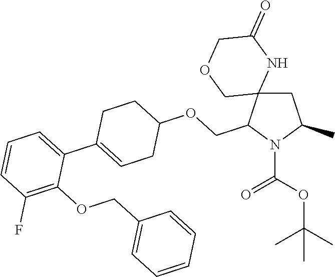 [SMILES: C[C@@H]1CC2(COCC(=O)N2)C(COC2CC=C(c3cccc(F)c3OCc3ccccc3)CC2)N1C(=O)OC(C)(C)C]

### tert-butyl-(3R)-1-[({4-[2-(benzyloxy)-3-fluorophenyl]cyclohex-3-en-1-yl}oxy)methyl]-3-methyl-7-oxo-9-oxa-2,6-diazaspiro[4.5]decane-2-carboxylate

A solution of Intermediate 59 (2.85 g), 2-benzyloxy-1-bromo-3-fluoro-benzene (1.50 g, 5.34 mmol) (1 {E},4 {E})-1,5-diphenylpenta-1,4-dien-3-one; palladium (231 mg, 0.252 mmol), dicyclohexyl-[2-(2,4,6-triisopropylphenyl)phenyl]phosphane (241 mg, 0.505 mmol) and lithium tert-butoxide (1.1 mL, 12.6 mmol) in anhydrous 1,4-dioxane (30 mL) degassed for 10 minutes then heated to 110° C. under a nitrogen atmosphere for 21 hours. The mixture was cooled to room temperature and filtered through Celite, washed with ethyl acetate. The filtrate was concentrated in vacuo to afford the crude material. The crude material was purified by silica gel column chromatography (0-100% EtOAc in heptane) to afford the title compound (2.32 g) as an orange oil. [M+Na]⁺ m/z 603.3

### Intermediate 61

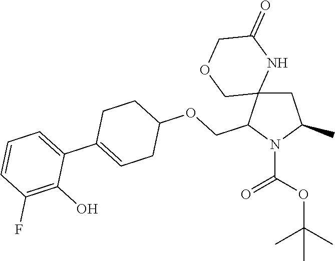 [SMILES: C[C@@H]1CC2(COCC(=O)N2)C(COC2CC=C(c3cccc(F)c3O)CC2)N1C(=O)OC(C)(C)C]

### tert-butyl-(3R)-1-({[4-(3-fluoro-2-hydroxyphenyl)cyclohex-3-en-1-yl]oxy}methyl)-3-methyl-7-oxo-9-oxa-2,6-diazaspiro[4.5]decane-2-carboxylate

Intermediate 60 (2.32 g) was dissolved in ethanol (78 mL) and the atmosphere was evacuated and backfilled with nitrogen 3 times. Palladium on carbon (10%, 850 mg, 0.799 mmol) was added and the atmosphere was evacuated and backfilled with hydrogen 3 times. The reaction was stirred for 2 hours and then filtered through Celite, washing with EtOAc and the filtrate was concentrated in vacuo to afford the title compound (2.1 g) as a light-yellow gum. [M+H]+ m/z 491.4.

### Intermediate 62

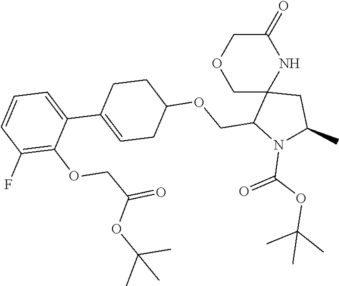 [SMILES: C[C@@H]1CC2(COCC(=O)N2)C(COC2CC=C(c3cccc(F)c3OCC(=O)OC(C)(C)C)CC2)N1C(=O)OC(C)(C)C]

### tert-butyl-(3R)-1-{[(4-{2-[2-(tert-butoxy)-2-oxoethoxy]-3-fluorophenyl}cyclohex-3-en-1-yl)oxy]methyl}-3-methyl-7-oxo-9-oxa-2,6-diazaspiro[4.5]decane-2-carboxylate

To a solution of Intermediate 61 (0.80 g) and tert-butyl bromoacetate (0.37 mL, 2.45 mmol) in acetone (8.6 mL) was added dipotassium carbonate (676 mg, 4.89 mmol) and the solution was heated to 50° C. overnight. The solids were filtered off and the filtrate was concentrated in vacuo. The residue was suspended in water (15 mL) and extracted with DCM (3×20 mL). The combined organic extracts were concentrated in vacuo to afford the title compound (1.2 g) as a yellow oil. [M+H]⁺ m/z 605.4.

### Intermediate 63

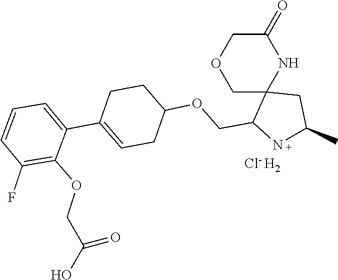

### (3R)-1-[({4-[2-(carboxymethoxy)-3-fluorophenyl]cyclohex-3-en-1-yl}oxy)methyl]-3-methyl-7-oxo-9-oxa-2,6-diazaspiro[4.5]decan-2-ium chloride

4 M Hydrogen chloride in dioxane (11 mL, 42.3 mmol) was added to Intermediate 62 (1.28 g) at room temperature and the reaction was stirred for 2 hours. The reaction mixture was concentrated in vacuo to afford the title compound (1.31 g) as a yellow solid. [M+H]⁺ m/z 449.3.

### Intermediate 64

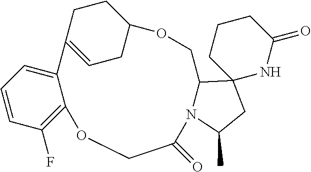 [SMILES: C[C@@H]1CC2(CCCC(=O)N2)C2COC3CC=C(CC3)c3cccc(F)c3OCC(=O)N21]

### (12′R)-6′-fluoro-12′-methyl-8′,17′-dioxa-11′-azaspiro[morpholine-3,14′tetracyclo[16.2.2.0^{2,7}0.0^{11,15}]docosane]-1′(20′),2′,4′,6′-tetraene-5,10′-dione

To a stirred solution of HATU (1.65 g, 4.35 mmol) and DIPEA (1.50 mL, 8.59 mmol) in acetonitrile (150 mL) was added Intermediate 63 (1.31 g) in DMF (10 mL) over 2h using a syringe pump. The resulting solution was stirred for one hour. The reaction mixture was concentrated in vacuo to afford the crude material. The crude material was diluted in water and extracted with DCM (3×20 mL) The combined organic extracts were dried (MgSO₄), filtered and concentrated to give a solid residue. The crude material was purified by basic reverse phase column chromatography (10-50% acetonitrile in water (0.1% ammonia)) to afford the title compound (350 mg) as a yellow oil. [M+H]⁺ m/z 431.3.

### Examples 15a and 15b

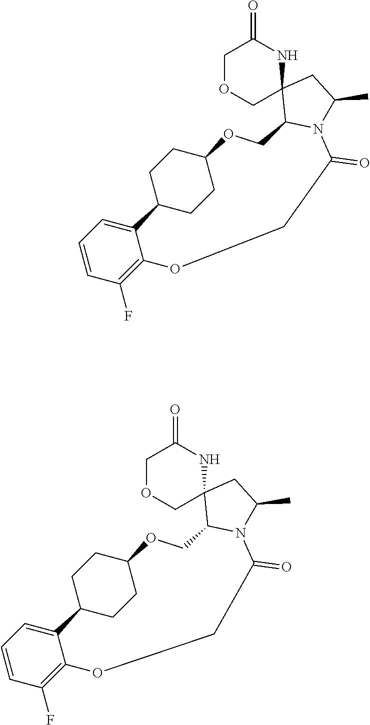 [SMILES: C[C@@H]1C[C@@]2(COCC(=O)N2)[C@@H]2CO[C@H]3CC[C@H](CC3)c3cccc(F)c3OCC(=O)N12 ; C[C@@H]1C[C@]2(COCC(=O)N2)[C@H]2CO[C@H]3CC[C@H](CC3)c3cccc(F)c3OCC(=O)N12]

### Example 15a: (1′s,3S,12′R,15′R,18′s)-6′-fluoro-12′-methyl-8′,17′-dioxa-11′-azaspiro[morpholine-3,14′-tetracyclo[16.2.2.0^{2,7}0.0^{11,15}]docosane]-2′,4′,6′-triene-5,10′-dione

### Example 15b: (1′s,3R,12′R,15′S,18′s)-6′-fluoro-12′-methyl-8′,17′-dioxa-11′-azaspiro[morpholine-3,14′-tetracyclo[16.2.2.0^{2,7}0.0^{11,15}]docosane]-2′,4′,6′-triene-5,10′-dione

Intermediate 64 (300 mg) was dissolved in ethanol (24 mL) and the atmosphere was evacuated and backfilled with nitrogen 3 times. Palladium on carbon (10%, 345 mg, 0.324 mmol) was added and the atmosphere was evacuated and backfilled with hydrogen 3 times. The reaction was stirred for 24 hours and then filtered through Celite, washing with EtOAc and the filtrate was concentrated in vacuo to afford the crude mixture. The crude material was purified by basic reverse phase column chromatography (15-50% acetonitrile in water (0.1% ammonia)) to afford the title compounds (Peak 1, 53 mg) as a white solid and (Peak 2, 30 mg) as a white solid.

### Example 15a: Peak 1

LCMS (Method A): [M+H]⁺ m/z 433.3, RT 2.99 min

¹H NMR (500 MHZ, CDCl₃) δ 7.42 (s, 1H), 6.96-6.90 (m, 1H), 6.87-6.79 (m, 2H), 5.22 (d, J=11.2 Hz, 1H), 4.52-4.13 (m, 6H), 3.88 (s, 1H), 3.75-3.59 (m, 2H), 3.25 (d, J=9.4 Hz, 1H), 2.87-2.71 (m, 1H), 2.66-2.50 (m, 1H), 2.35 (dd, J=13.4, 8.3 Hz, 1H), 2.24-2.12 (m, 2H), 2.07-2.01 (m, 1H), 1.99-1.94 (m, 1H), 1.68 (d, J=6.9 Hz, 3H), 1.50-1.37 (m, 3H), 1.37-1.29 (m, 1H).

### Example 15b: Peak 2

LCMS (Method A): [M+H]⁺ m/z 433.3, RT 3.29 min

¹H NMR (500 MHZ, CDCl₃) δ 7.01-6.82 (m, 3H), 6.22 (s, 1H), 5.36 (dd, J=12.7, 3.7 Hz, 1H), 4.87 (ddt, J=12.3, 7.7, 3.8 Hz, 1H), 4.53 (d, J=2.4 Hz, 1H), 4.32 (dd, J=9.9, 2.8 Hz, 1H), 4.26 (d, J=16.7 Hz, 1H), 4.22 (dd, J=12.7, 2.3 Hz, 1H), 4.14 (d, J=16.7 Hz, 1H), 4.02 (d, J=11.6 Hz, 1H), 3.84 (s, 1H), 3.62 (d, J=11.6 Hz, 1H), 3.25 (dd, J=9.8, 1.1 Hz, 1H), 2.66-2.39 (m, 3H), 2.15 (d, J=14.7 Hz, 1H), 1.92-1.72 (m, 3H), 1.49 (d, J=12.3 Hz, 3H), 1.43 (d, J=6.6 Hz, 3H), 1.34 (td, J=14.2, 3.9 Hz, 1H).

Below examples were prepared following analogous procedures as described for Examples 15a and 15b using the appropriate reagents followed by purification.

| | | | Obs. | |
|---|---|---|---|---|
| Ex. | Structure | Name | Mass | ¹H NMR |
| 16a | 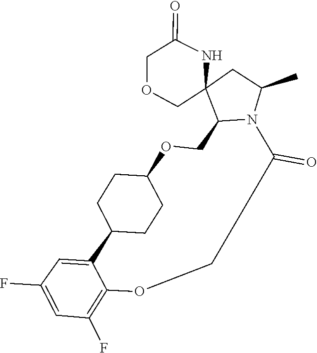 [SMILES: C[C@@H]1C[C@@]2(COCC(=O)N2)[C@@H]2CO[C@H]3CC[C@H](CC3)c3cc(F)cc(F)c3OCC(=O)N12] | (1′s,3S,12′R,15′R, 18′s)-4′,6′- difluoro-12′- methyl-8′,17′- dioxa-11′- azaspiro [morpholine- 3,14′- tetracyclo[16.2.2. 0^{2,7}.0¹¹,¹⁵] docosane]- 2′,4′,6′-triene- 5,10′-dione | LCMS (Method C): [M + H]+ m/z 451.3, RT 0.96 min | ¹H NMR (500 MHz, CDCl₃) δ 7.39 (br s, 1 H), 6.72 (ddd, J = 11.5, 8.2, 3.0 Hz, 1 H),6.59 − 6.66 (m, 1 H), 5.21 (br d, J = 9.5 Hz, 1 H), 4.32 − 4.47 (m, 2 H), 4.19 − 4.32 (m, 3 H), 4.11 − 4.19 (m, 1H), 3.84 − 3.94 (m, 1 H), 3.63 − 3.73 (m, 2 H), 3.27 (br d, J = 9.1 Hz, 1 H), 2.78 (qd, J = 12.9, 4.3 Hz, 1 H), 2.47 − 2.59 (m, 1 H), 2.35 (dd, J = 13.4, 8.3 Hz, 1 H), 2.12 − 2.29 (m, 2 H), 1.92 − 2.09 (m, 2 H), 1.68 (br d, J = 6.3 Hz, 3 H), 1.27 − 1.56 (m, 4H) |
| 16b | 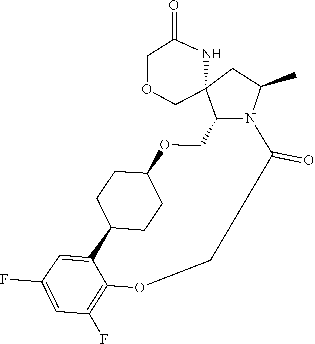 [SMILES: C[C@@H]1C[C@]2(COCC(=O)N2)[C@H]2CO[C@H]3CC[C@H](CC3)c3cc(F)cc(F)c3OCC(=O)N12] | (1′s,3R,12′R,15′S, 18′s)-4′,6′- difluoro-12′- methyl-8′,17′- dioxa-11′- azaspiro [morpholine- 3,14′- tetracyclo [16.2.2.0^{2,7}.0^{11,15}] docosane]- 2′,4′,6′-triene- 5,10′-dione | LCMS (Method A): [M + H]+ m/z 451.3, RT 3.3 8 minutes. | ¹H NMR (500 MHz, CDCl₃) δ 6.71 (ddd, J = 11.2, 8.0, 3.1 Hz, 1H), 6.61 (ddd, J = 8.8, 3.1, 1.8 Hz, 1H), 6.31 (s, 1H), 5.33 (dd, J = 12.7, 3.5 Hz, 1H), 4.89 − 4.80 (m, 1H), 4.52 (d, J = 2.6 Hz, 1H), 4.30 (dd, J = 9.9, 2.8 Hz, 1H), 4.24 (d, J = 16.7 Hz, 1H), 4.19 − 4.09 (m, 2H), 4.00 (d, J = 11.6 Hz, 1H), 3.82 (p, J = 2.2 Hz, 1H), 3.61 (d, J = 11.6 Hz, 1H), 3.24 (dd, J = 9.9, 1.1 Hz, 1H), 2.57 (dd, J = 13.1, 9.7 Hz, 1H), 2.51 − 2.39 (m, 2H), 2.15 (ddt, J = 14.8, 6.5, 2.9 Hz, 1H), 1.90 − 1.77 (m, 3H), 1.53 − 1.43 (m, 3H), 1.41 (d, J = 6.6 Hz, 3H), 1.38 − 1.27 (m, 1H). |

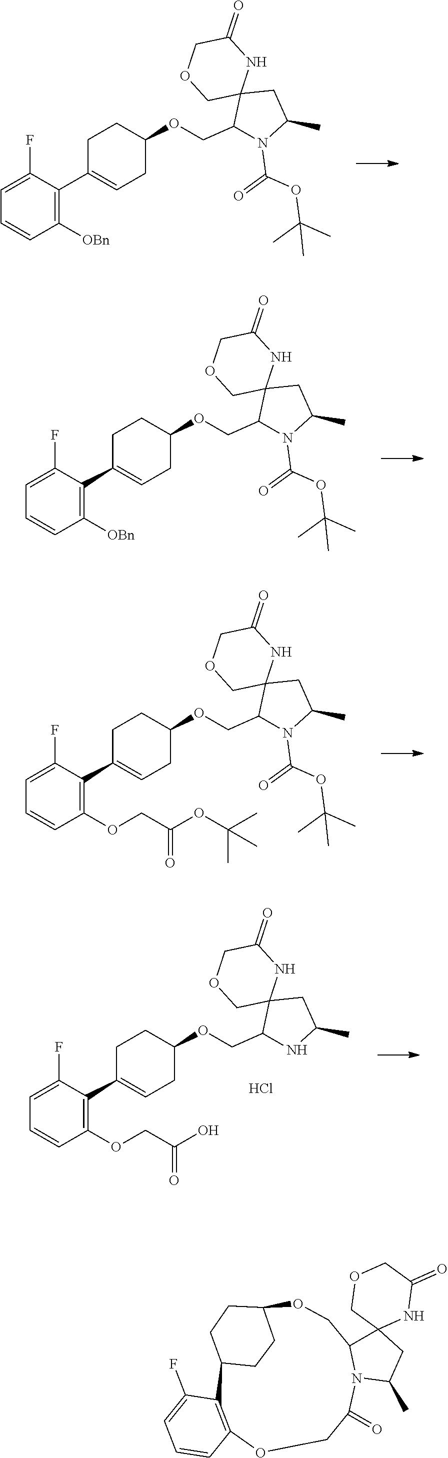

### Intermediate 65

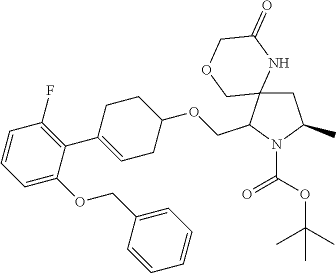 [SMILES: C[C@@H]1CC2(COCC(=O)N2)C(COC2CC=C(c3c(F)cccc3OCc3ccccc3)CC2)N1C(=O)OC(C)(C)C]

### tert-butyl-(3R)-1-[({4-[2-(benzyloxy)-6-fluorophenyl]cyclohex-3-en-1-yl}oxy)methyl]-3-methyl-7-oxo-9-oxa-2,6-diazaspiro[4.5]decane-2-carboxylate

Intermediate 65 was prepared using Intermediate 59 and known starting material 1-(benzyloxy)-2-bromo-3-fluorobenzene following a similar procedure as described for Intermediate 60 to afford the title compound (2.75 g) as a yellow oil. [M+H]⁺ m/z 581.6.

### Intermediate 66

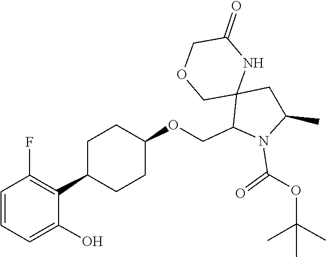 [SMILES: C[C@@H]1CC2(COCC(=O)N2)C(CO[C@H]2CC[C@@H](c3c(O)cccc3F)CC2)N1C(=O)OC(C)(C)C]

### tert-butyl-(3R)-3-methyl-7-oxo-1-({[(1s,4s)-4-(2-fluoro-6-hydroxyphenyl)cyclohexyl]-oxy}methyl)-9-oxa-2,6-diazaspiro[4.5]decane-2-carboxylate

Intermediate 65 (330 mg) was dissolved in ethanol (11.095 mL) and the atmosphere was evacuated and backfilled with nitrogen 3 times. Palladium on carbon (10%, 242 mg, 0.227 mmol) was added and the atmosphere was evacuated and backfilled with hydrogen 3 times. The reaction was stirred for 16 hours and then filtered through Celite, washing with EtOAc and the filtrate was concentrated in vacuo to afford the title compound (270 mg) as a yellow oil. [M+H]⁺ m/z 493.4.

### Intermediate 67

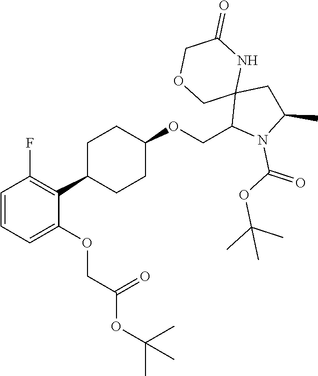 [SMILES: C[C@@H]1CC2(COCC(=O)N2)C(CO[C@H]2CC[C@@H](c3c(F)cccc3OCC(=O)OC(C)(C)C)CC2)N1C(=O)OC(C)(C)C]

### tert-butyl (3R)-3-methyl-7-oxo-1-({[(1s,4s)-4-{2-[2-(tert-butoxy)-2-oxoethoxy]-6-fluorophenyl}cyclohexyl]oxy}methyl)-9-oxa-2,6-diazaspiro[4.5]decane-2-carboxylate

To a solution of Intermediate 66 (270 mg) and tert-butyl 2-bromoacetate (209 mg, 1.05 mmol) in acetonitrile (6.7 mL) was added dipotassium carbonate (145 mg, 1.05 mmol) and the solution was heated to 50° C. for 3 hours. The solids were filtered off and the filtrate was concentrated in vacuo. The residue was suspended in water (5 mL) and extracted with DCM (3×10 mL). The combined organic extracts were concentrated in vacuo to afford the title compound (250 mg) as a yellow oil. [M+H]⁺ m/z 607.5.

### Intermediate 68

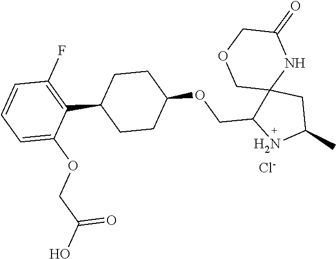 [SMILES: C[C@@H]1CC2(COCC(=O)N2)C(CO[C@H]2CC[C@@H](c3c(F)cccc3OCC(=O)O)CC2)N1 ; [Cl-]]

### (3R)-3-methyl-7-oxo-1-({[(1s,4s)-4-[2-(carboxymethoxy)-6-fluorophenyl]cyclohexyl]-oxy}methyl)-9-oxa-2,6-diazaspiro[4.5]decan-2-ium chloride

4 M Hydrogen chloride in dioxane (1.6 mL, 6.59 mmol) was added to Intermediate 67 (250 mg) at room temperature and the reaction was stirred for 2 hours. The reaction mixture was concentrated in vacuo to afford the title compound (250 mg) as a yellow solid. [M+H]⁺ m/z 451.3

### Examples 17a and 17b

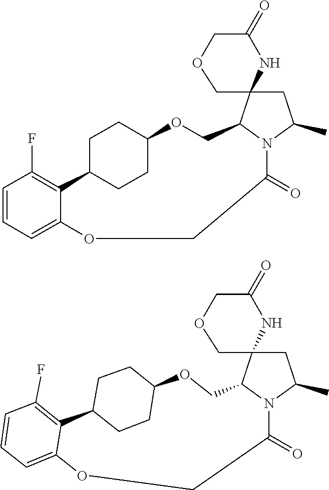 [SMILES: C[C@@H]1C[C@@]2(COCC(=O)N2)[C@@H]2CO[C@H]3CC[C@H](CC3)c3c(F)cccc3OCC(=O)N12 ; C[C@@H]1C[C@]2(COCC(=O)N2)[C@H]2CO[C@H]3CC[C@H](CC3)c3c(F)cccc3OCC(=O)N12]

### Example 17a: (1′s,3S,12′R,15′R,18′s)-3′-fluoro-12′-methyl-8′,17′-dioxa-11′-azaspiro[morpholine-3,14′-tetracyclo[16.2.2.0^{2,7}0.0^{11,15}]docosane]-2′,4′,6′-triene-5,10′-dione

### Example 17b: (1′s,3R,12′R,15′S, 18′s)-3′-fluoro-12′-methyl-8′,17′-dioxa-11′-azaspiro[morpholine-3,14′-tetracyclo[16.2.2.0^{2,7}0.0^{11,15}]docosane]-2′,4′,6′-triene-5,10′-dione

To a stirred solution of HATU (201 mg, 0.529 mmol) and DIPEA (182 μL, 1.04 mmol) in acetonitrile (24.3 mL) was added Intermediate 68 (250 mg) in DMF (2.4 mL) over 2h using a syringe pump. The resulting solution was stirred for one hour. The reaction mixture was concentrated in vacuo to afford the crude material. The mixture was diluted in water and extracted with DCM (3×5 mL) The combined organic extracts were dried (MgSO₄), filtered and concentrated to give a solid residue. The crude material was purified by basic reverse phase column chromatography (15-45% acetonitrile in water (0.1% ammonia)) to afford the title compounds (Peak 1, 8.6 mg) as a white solid and (Peak 2, 11.3 mg) as a white solid.

### Example 17a: Peak 1

LCMS (Method A): [M+H]⁺ m/z 433.3, RT 3.03 min

¹H NMR (500 MHZ, CDCl₃) δ 7.47 (s, 1H), 7.12-7.05 (m, 1H), 6.74-6.65 (m, 1H), 6.60-6.49 (m, 1H), 5.02-4.91 (m, 1H), 4.41-4.15 (m, 5H), 3.88 (s, 1H), 3.76-3.56 (m, 2H), 3.29-3.11 (m, 2H), 2.80-2.62 (m, 1H), 2.44-2.25 (m, 2H), 2.21-2.07 (m, 3H), 2.00-1.88 (m, 1H), 1.44-1.37 (m, 3H), 1.32-1.28 (m, 4H).

### Example 17b: Peak 2

LCMS (Method A): [M+H]⁺ m/z 433.3, RT 3.23 min

¹H NMR (500 MHZ, CDCl₃) δ 7.17-7.02 (m, 1H), 6.71 (t, J=8.8 Hz, 1H), 6.55 (d, J=8.1 Hz, 1H), 6.28 (s, 1H), 5.06 (d, J=10.3 Hz, 1H), 4.73-4.60 (m, 1H), 4.55 (s, 1H), 4.39-4.21 (m, 3H), 4.13 (d, J=16.8 Hz, 1H), 4.03 (d, J=11.5 Hz, 1H), 3.91-3.74 (m, 1H), 3.60 (d, J=11.6 Hz, 1H), 3.24 (d, J=9.8 Hz, 1H), 3.19-3.07 (m, 1H), 2.74-2.59 (m, 1H), 2.55-2.33 (m, 1H), 2.17-2.01 (m, 2H), 1.90-1.75 (m, 2H), 1.59-1.48 (m, 1H), 1.47-1.29 (m, 6H).

Below examples were prepared following analogous procedures as described for Example 17a and17b using the appropriate reagents

| Ex. | Structure | Name | Obs. Mass | ¹H NMR |
|---|---|---|---|---|
| 18a | 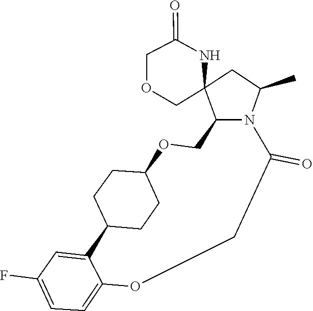 [SMILES: C[C@@H]1C[C@@]2(COCC(=O)N2)[C@@H]2CO[C@H]3CC[C@H](CC3)c3cc(F)ccc3OCC(=O)N12] | (1′s,3S,12′R,15′R, 18′s)-4′-fluoro-12′- methyl-8′,17′- dioxa-11′- azaspiro [morpholine-3,14′- tetracyclo [16.2.2.0²,⁷.0¹¹,¹⁵] docosane]- 2′,4′,6′-triene-5,10′- dione | LCMS (Method B): [M + H]⁺ m/z 433.3, RT 2.91 minutes. | ¹H NMR (400 MHz, CDCl₃) δ 7.44 (s, 1H), 6.90 − 6.76 (m, 2H), 6.70 (s, 1H), 4.97 (s, 1H), 4.44 − 4.10 (m, 5H), 3.87 (s, 1H), 3.75 − 3.59 (m, 2H), 3.24 (s, 1H), 2.59 − 2.42 (m, 1H), 2.41 − 2.28 (m, 1H), 2.24 − 2.02 (m, 3H), 1.98 − 1.89 (m, 1H), 1.63 (s, 3H), 1.50 − 1.27 (m, 6H). |
| 18b | 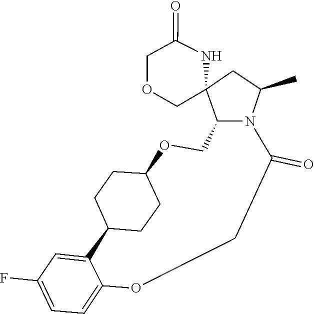 [SMILES: C[C@@H]1C[C@]2(COCC(=O)N2)[C@H]2CO[C@H]3CC[C@H](CC3)c3cc(F)ccc3OCC(=O)N12] | (1′s,3R,12′R,15′S, 18′s)-4′-fluoro-12′- methyl-8′,17′- dioxa-11′- azaspiro[morpholine- 3,14′- tetracyclo [16.2.2.0²,⁷.0¹¹,¹⁵] docosane]- 2′,4′,6′-triene-5,10′- dione | LCMS (Method B): [M + H]⁺ m/z 433.2, RT 3.03 minutes. | ¹H NMR (400 MHz, CDCl₃) δ 6.92 − 6.77 (m, 2H), 6.70 (dd, J = 8.7, 4.6 Hz, 1H), 6.18 (s, 1H), 5.07 (d, J = 10.7 Hz, 1H), 4.70 (dt, J = 13.9, 6.8 Hz, 1H), 4.54 (s, 1H), 4.33 − 4.18 (m, 3H), 4.13 (d, J = 16.8 Hz, 1H), 4.03 (d, J = 11.6 Hz, 1H), 3.84 (s, 1H), 3.61 (d, J = 11.6 Hz, 1H), 3.24 (d, J = 9.8 Hz, 1H), 2.62 (dd, J = 12.9, 9.9 Hz, 1H), 2.48 (s, 2H), 2.15 − 1.96 (m, 2H), 1.86 − 1.76 (m, 2H), 1.49 (s, 2H), 1.43 − 1.25 (m, 5H). |
| 19a | 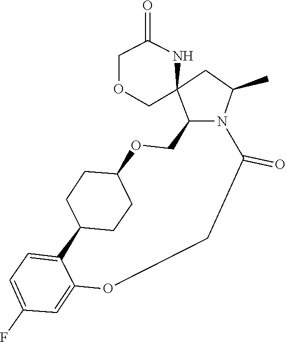 [SMILES: C[C@@H]1C[C@@]2(COCC(=O)N2)[C@@H]2CO[C@H]3CC[C@H](CC3)c3ccc(F)cc3OCC(=O)N12] | (1′s,3S,12′R,15′R, 18′s)-5′-fluoro-12′- methyl-8′,17′- dioxa-11′- azaspiro[morpholine- 3,14′- tetracyclo [16.2.2.0²,⁷.0¹¹,¹⁵] docosane]- 2′,4′,6′-triene-5,10′- dione | LCMS (Method A): [M + H]⁺ m/z 433.3, RT 3.16 minutes. | ¹H NMR (500 MHz, CDCl₃) δ 7.44 (s, 1H), 7.11 − 6.94 (m, 1H), 6.63 − 6.53 (m, 1H), 6.52 − 6.43 (m, 1H), 5.04 − 4.85 (m, 1H), 4.41 − 4.16 (m, 5H), 3.87 (s, 1H), 3.76 − 3.59 (m, 2H), 3.29 − 3.12 (m, 1H), 2.76 − 2.62 (m, 1H), 2.55 (s, 1H), 2.43 − 2.30 (m, 1H), 2.21 − 2.04 (m, 3H), 1.98 − 1.90 (m, 1H), 1.52 − 1.39 (m, 5H), 1.38 − 1.26 (m, 3H) |
| 19b | 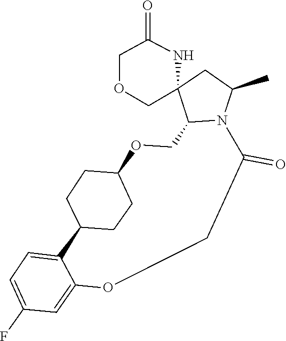 | (1′s,3R,12′R,15′S, 18′s)-5′-fluoro-12′- methyl-8′,17′- dioxa-11′- azaspiro[morpholine- 3,14′- tetracyclo [16.2.2.0²,⁷.0¹¹,¹⁵] docosane]- 2′,4′,6′-triene-5,10′- dione | LCMS (Method A): [M + H]⁺ m/z 433.3, RT 3.37 minutes. | ¹H NMR (400 MHz, CDCl₃) δ 7.02 (t, J = 7.6 Hz, 1H), 6.70 − 6.54 (m, 1H), 6.50 (dd, J = 10.0, 2.6 Hz, 1H), 6.19 (s, 1H), 5.04 (d, J = 10.0 Hz, 1H), 4.71 − 4.58 (m, 1H), 4.55 (s, 1H), 4.37 − 4.20 (m, 3H), 4.18 − 4.10 (m, 1H), 4.04 (d, J = 11.5 Hz, 1H), 3.83 (s, 1H), 3.60 (d, J = 11.5 Hz, 1H), 3.24 (d, J = 9.9 Hz, 1H), 2.70 − 2.60 (m, 1H), 2.58 − 2.37 (m, 2H), 2.12 − 1.95 (m, 2H), 1.87 − 1.74 (m, 2H), 1.56 − 1.43 (m, 2H), 1.41 − 1.32 (m, 5H). |
| 20a | 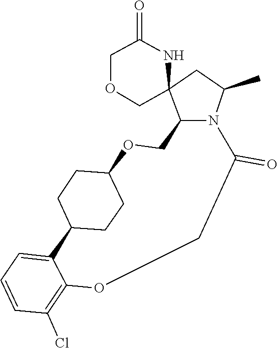 [SMILES: C[C@@H]1C[C@@]2(COCC(=O)N2)[C@@H]2CO[C@H]3CC[C@H](CC3)c3cccc(Cl)c3OCC(=O)N12] | (1′s,3S,12′R,15′R, 18′s)-6′-chloro-12′- methyl-8′,17′- dioxa-11′- azaspiro[morpholine- 3,14′- tetracyclo [16.2.2.0²,⁷.0¹¹,¹⁵] docosane]- 2′,4′,6′-triene-5,10′- dione | LCMS (Method A): [M + H]⁺ m/z 449.3, RT 3.03 minutes. | ¹H NMR (400 MHz, CDCl₃) δ 7.21 (dd, J = 7.9, 1.7 Hz, 1H), 7.06 − 6.96 (m, 2H), 6.90 (t, J = 7.7 Hz, 1H), 5.48 − 5.34 (m, 1H), 4.49 − 4.06 (m, 8H), 4.05 − 3.90 (m, 1H), 3.91 − 3.85 (m, 1H), 3.66 (s, 1H), 3.33 − 3.25 (m, 1H), 2.86 − 2.78 (m, 1H), 2.64 − 2.47 (m, 2H), 2.35 (dd, J = 13.4, 8.3 Hz, 1H), 2.17 (d, J = 15.1 Hz, 1H), 2.05 − 1.93 (m, 2H), 1.70 (d, J = 6.9 Hz, 3H), 1.44 − 1.23 (m, 2H). |
| 20b | 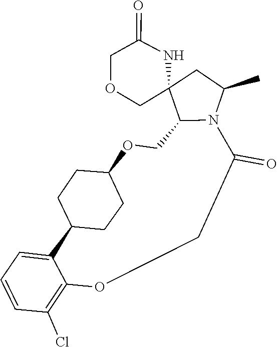 [SMILES: C[C@@H]1C[C@]2(COCC(=O)N2)[C@H]2CO[C@H]3CC[C@H](CC3)c3cccc(Cl)c3OCC(=O)N12] | (1′s,3R,12′R,15′S, 18′s)-6′-chloro-12′- methyl-8′,17′- dioxa-11′- azaspiro[morpholine- 3,14′- tetracyclo [16.2.2.0²,⁷.0¹¹,¹⁵] docosane]- 2′(7′),3′,5′-triene- 5,10′-dione | LCMS (Method A): [M + H]⁺ m/z 449.3, RT 3.38 minutes. | ¹H NMR (400 MHz, CDCl₃) δ 7.22 (dd, J = 7.9, 1.8 Hz, 1H), 7.02 (dd, J = 7.6, 1.8 Hz, 1H), 6.94 (t, J = 7.7 Hz, 1H), 6.28 (s, 1H), 5.65 (d, J = 13.3 Hz, 1H), 5.01 − 4.90 (m, 1H), 4.55 (d, J = 2.6 Hz, 1H), 4.30 (dd, J = 9.9, 2.9 Hz, 1H), 4.25 (d, J = 16.8 Hz, 1H), 4.15 (d, J = 16.8 Hz, 1H), 4.08 (d, J = 13.3 Hz, 1H), 3.99 (d, J = 11.6 Hz, 1H), 3.84 (s, 1H), 3.63 (d, J = 11.6 Hz, 1H), 3.27 (d, J = 9.8 Hz, 1H), 2.61 − 2.38 (m, 3H), 2.17 (d, J = 14.9 Hz, 1H), 1.91 − 1.74 (m, 4H), 1.52 − 1.41 (m, 6H). |
| 21a | 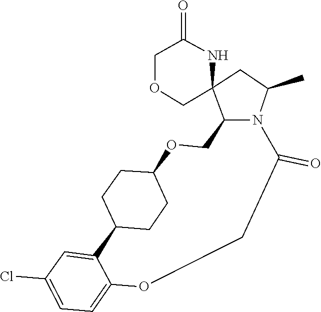 [SMILES: C[C@@H]1C[C@@]2(COCC(=O)N2)[C@@H]2CO[C@H]3CC[C@H](CC3)c3cc(Cl)ccc3OCC(=O)N12] | (1′s,3S,12′R,15′R, 18′s)-4′-chloro-12′- methyl-8′,17′- dioxa-11′- azaspiro [morpholine- 3,14′- tetracyclo [16.2.2.0²,⁷.0¹¹,¹⁵] docosane]- 2′,4′,6′-triene-5,10′- dione | LCMS (Method B): [M + H]⁺ m/z 449.2, RT 3.10 minutes. | ¹H NMR (500 MHz, CDCl₃) δ 7.42 (s, 1H), 7.12 (dd, J = 8.5, 2.6 Hz, 1H), 7.06 (d, J = 2.7 Hz, 1H), 6.68 (d, J = 8.5 Hz, 1H), 4.97 (d, J = 9.4 Hz, 1H), 4.43 − 4.34 (m, 1H), 4.33 − 4.25 (m, 2H), 4.24 − 4.16 (m, 2H), 3.91 − 3.80 (m, 1H), 3.72 − 3.61 (m, 2H), 3.22 (d, J = 9.4 Hz, 1H), 2.80 − 2.67 (m, 1H), 2.58 − 2.46 (m, 1H), 2.41 − 2.28 (m, 1H), 2.22 − 2.05 (m, 3H), 1.94 (d, J = 13.8 Hz, 1H), 1.61 (d, J = 6.8 Hz, 3H), 1.56 − 1.41 (m, 3H), 1.34 (d, J = 13.2 Hz, 2H). |
| 21b | 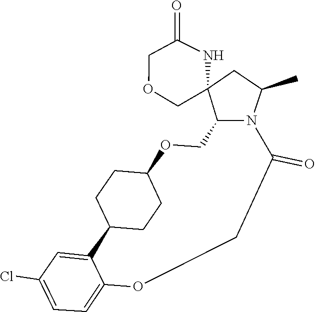 [SMILES: C[C@@H]1C[C@]2(COCC(=O)N2)[C@H]2CO[C@H]3CC[C@H](CC3)c3cc(Cl)ccc3OCC(=O)N12] | (1′s,3R,12′R,15′S, 18′s)-4′-chloro-12′- methyl-8′,17′- dioxa-11′- azaspiro[morpholine- 3,14′- tetracyclo [16.2.2.0²,⁷.0¹¹,¹⁵] docosane]- 2′(7′),3′,5′-triene- 5,10′-dione | LCMS (Method B): [M + H]⁺ m/z 449.3, RT 3.32 minutes. | ¹H NMR (500 MHz, CDCl₃) δ 7.12 (dd, J = 8.5, 2.6 Hz, 1H), 7.07 (d, J = 2.6 Hz, 1H), 6.68 (d, J = 8.6 Hz, 1H), 6.25 (s, 1H), 5.06 (d, J = 10.4 Hz, 1H), 4.64 (dtd, J = 16.5, 6.5, 1.5 Hz, 1H), 4.54 (d, J = 2.2 Hz, 1H), 4.29 (dd, J = 9.9, 2.6 Hz, 1H), 4.27 − 4.18 (m, 2H), 4.12 (d, J = 16.7 Hz, 1H), 4.03 (dt, J = 11.6, 1.0 Hz, 1H), 3.83 (dt, J = 4.2, 2.1 Hz, 1H), 3.59 (d, J = 11.6 Hz, 1H), 3.23 (dd, J = 9.8, 1.2 Hz, 1H), 2.63 (dd, J = 13.0, 9.8 Hz, 1H), 2.55 − 2.40 (m, 2H), 2.13 − 2.00 (m, 2H), 1.84 − 1.75 (m, 2H), 1.52 − 1.43 (m, 2H), 1.38 (d, J = 6.6 Hz, 3H), 1.37 − 1.28 (m, 2H). |
| 22a | 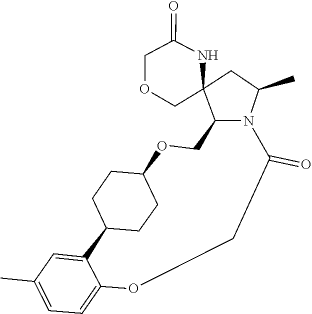 [SMILES: Cc1ccc2c(c1)[C@H]1CC[C@H](CC1)OC[C@@H]1N(C(=O)CO2)[C@H](C)C[C@@]12COCC(=O)N2] | (1′s,3S,12′R,15′R, 18′s)-4′,12′- dimethyl-8′,17′- dioxa-11′- azaspiro [morpholine- 3,14′- tetracyclo [16.2.2.0²,⁷.0¹¹,¹⁵] docosane]- 2′,4′,6′-triene-5,10′- dione | LCMS (Method B): [M + H]⁺ m/z 429.3, RT 2.85 minutes. | ¹H NMR (400 MHz, CDCl₃) δ 7.44 (d, J = 21.6 Hz, 1H), 6.99 − 6.92 (m, 1H), 6.89 (d, J = 2.3 Hz, 1H), 6.66 (s, 1H), 4.95 (s, 1H), 4.43 − 4.13 (m, 5H), 3.87 (s, 1H), 3.65 (d, J = 11.0 Hz, 2H), 3.22 (d, J = 9.4 Hz, 1H), 2.76 (d, J = 13.7 Hz, 1H), 2.50 (s, 1H), 2.35 (dd, J = 13.4, 8.3 Hz, 1H), 2.25 (s, 3H), 2.21 − 2.05 (m, 3H), 1.94 (d, J = 13.8 Hz, 1H), 1.62 (s, 2H), 1.52 − 1.23 (m, 6H). |
| 22b | 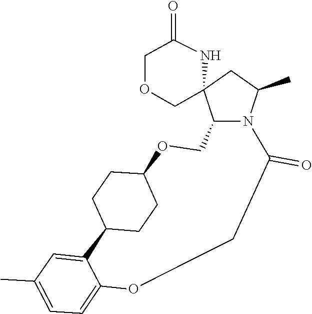 [SMILES: Cc1ccc2c(c1)[C@H]1CC[C@H](CC1)OC[C@H]1N(C(=O)CO2)[C@H](C)C[C@]12COCC(=O)N2] | (1′s,3R,12′R,15′S, 18′s)-4′,12′- dimethyl-8′,17′- dioxa-11′- azaspiro[morpholine- 3,14′- tetracyclo [16.2.2.0²,⁷.0¹¹,¹⁵] docosane]- 2′,4′,6′-triene-5,10′- dione | LCMS (Method B): [M + H]⁺ m/z 429.3, RT 3.03 minutes. | ¹H NMR (400 MHz, CDCl₃) δ 6.96 (ddd, J = 8.1, 2.3, 0.8 Hz, 1H), 6.89 (d, J = 2.3 Hz, 1H), 6.67 (d, J = 8.1 Hz, 1H), 6.16 (s, 1H), 5.07 (d, J = 10.7 Hz, 1H), 4.72 (dtd, J = 9.4, 7.2, 5.6 Hz, 1H), 4.54 (d, J = 2.2 Hz, 1H), 4.30 (dd, J = 9.9, 2.5 Hz, 1H), 4.28 − 4.20 (m, 2H), 4.13 (d, J = 16.7 Hz, 1H), 4.06 − 4.00 (m, 1H), 3.83 (d, J = 4.2 Hz, 1H), 3.60 (d, J = 11.6 Hz, 1H), 3.24 (dd, J = 9.8, 1.2 Hz, 1H), 2.63 (dd, J = 13.0, 9.8 Hz, 1H), 2.53 − 2.43 (m, 2H), 2.26 (s, 3H), 2.14 − 1.98 (m, 2H), 1.80 (ddd, J = 14.9, 11.8, 2.3 Hz, 2H), 1.54 − 1.42 (m, 3H), 1.41 − 1.30 (m, 4H). |
| 23a | 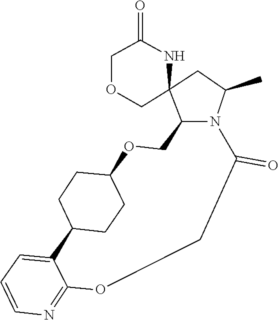 [SMILES: C[C@@H]1C[C@@]2(COCC(=O)N2)[C@@H]2CO[C@H]3CC[C@H](CC3)c3cccnc3OCC(=O)N12] | (1′s,3S,12′R,15′R, 18′s)-12′-methyl- 8′,17′-dioxa-6′,11′- diazaspiro [morpholine-3,14′- tetracyclo [16.2.2.0²,⁷.0¹¹,¹⁵] docosane]- 2′,4′,6′-triene-5,10′- dione | LCMS (Method B): [M + H]+ m/z 416.3, RT 2.34 minutes. | ¹H NMR (500 MHz, CDCl₃) δ 8.00 (dd, J = 5.0, 1.9 Hz, 1H), 7.43 (s, 1H), 7.37 (dd, J = 7.0, 2.0 Hz, 1H), 6.87 − 6.79 (m, 1H), 4.98 − 4.81 (m, 2H), 4.42 − 4.31 (m, 2H), 4.28 − 4.17 (m, 3H), 3.88 (s, 1H), 3.74 − 3.61 (m, 3H), 3.31 − 3.19 (m, 1H), 2.82 − 2.73 (m, 1H), 2.65 − 2.52 (m, 1H), 2.36 (dd, J = 12.7, 8.1 Hz, 1H), 2.11 (d, J = 20.5 Hz, 3H), 1.64 − 1.60 (m, 2H), 1.41 − 1.33 (m, 3H), 1.32 − 1.26 (m, 2H). |
| 23b | 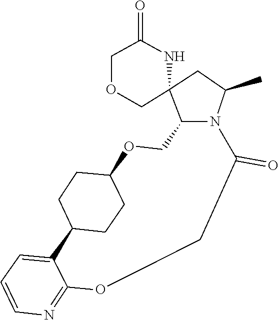 [SMILES: C[C@@H]1C[C@]2(COCC(=O)N2)[C@H]2CO[C@H]3CC[C@H](CC3)c3cccnc3OCC(=O)N12] | (1′s,3R,12′R, 15′S, 18′s)-12′-methyl- 8′,17′-dioxa-6′,11′- diazaspiro [morpholine-3,14′- tetracyclo [16.2.2.0²,⁷.0¹¹,¹⁵] docosane]- 2′,4′,6′-triene-5,10′- dione | LCMS (Method B): [M + H]+ m/z 416.3, RT 2.46 minutes. | ¹H NMR (500 MHz, CDCl₃) δ 8.00 (dd, J = 5.0, 1.9 Hz, 1H), 7.38 (dd, J = 7.2, 1.9 Hz, 1H), 6.85 (dd, J = 7.2, 5.0 Hz, 1H), 6.19 (s, 1H), 5.04 (d, J = 10.8 Hz, 1H), 4.69 (d, J = 10.9 Hz, 1H), 4.61 − 4.52 (m, 2H), 4.32 (dd, J = 9.8, 1.2 Hz, 1H), 2.66 (dd, J = 13.0, Hz, 1H), 4.13 (d, J = 16.7 Hz, 1H), 4.05 (dt, J = 11.5, 1.0 Hz, 1H), 3.88 − 3.81 (m, 1H), 3.80 − 3.62 (m, 1H), 3.60 (d, J = 11.6 Hz, 1H), 3.24 (dd, J = 9.8, 2.5 Hz, 1H), 4.26 (d, J = 16.7 9.8 Hz, 1H), 2.59 − 2.45 (m, 2H), 2.19 − 2.13 (m, 1H), 2.10 − 2.05 (m, 1H), 1.85 − 1.78 (m, 2H), 1.54 − 1.49 (m, 1H), 1.38 (d, J = 6.6 Hz, 3H), 1.35 − 1.33 (m, 1H), 1.33 − 1.30 (m, 1H). |

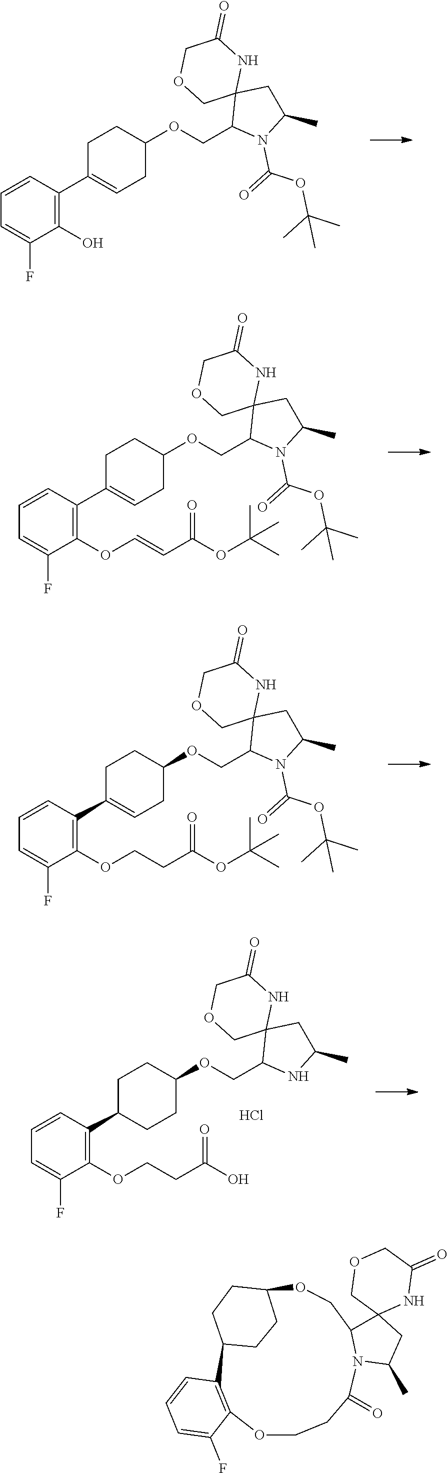

### Intermediate 69

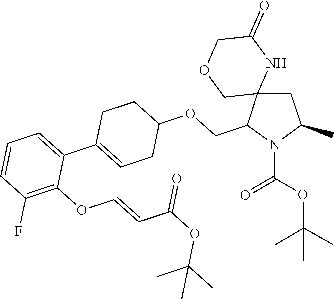 [SMILES: C[C@@H]1CC2(COCC(=O)N2)C(COC2CC=C(c3cccc(F)c3O/C=C/C(=O)OC(C)(C)C)CC2)N1C(=O)OC(C)(C)C]

### tert-butyl (3R)-1-({[4-(2-{[(1E)-3-(tert-butoxy)-3-oxoprop-1-en-1-yl]oxy}-3-fluorophenyl)cyclohex-3-en-1-yl]oxy}methyl)-3-methyl-7-oxo-9-oxa-2,6-diazaspiro[4.5]decane-2-carboxylate

To a solution of tert-butyl prop-2-ynoate (436 μL, 3.18 mmol) and 1,4-diazabicyclo[2.2.2]octane (117 mg, 1.04 mmol) in THF (2.1 mL) at 0° C. under nitrogen was added Intermediate 61 (1.30 g) in THF (11 mL) and the solution was stirred at room temperature for 5 hours. The reaction mixture was quenched with water (20 mL) and extracted with EtOAc (2×25 mL). The combined organic phases were washed with brine, dried over MgSO₄ and concentrated in vacuo to afford the crude. The crude was purified by silica gel column chromatography (0-80% EtOAc in heptane) to afford the title compound (1.48 g) as a yellow oil. [M+H]⁺ m/z 617.5.

### Intermediate 70

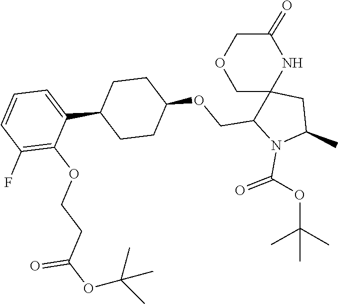 [SMILES: C[C@@H]1CC2(COCC(=O)N2)C(CO[C@H]2CC[C@@H](c3cccc(F)c3OCCC(=O)OC(C)(C)C)CC2)N1C(=O)OC(C)(C)C]

### tert-butyl (3R)-3-methyl-7-oxo-1-({[(1s,4s)-4-{2-[3-(tert-butoxy)-3-oxopropoxy]-3-fluorophenyl}cyclohexyl]oxy}methyl)-9-oxa-2,6-diazaspiro[4.5]decane-2-carboxylate

Intermediate 69 (1.48 g) was dissolved in ethanol (40 mL) and the atmosphere was evacuated and backfilled with nitrogen 3 times. Palladium on carbon (10%, 255 mg, 0.240 mmol) was added and the atmosphere was evacuated and backfilled with hydrogen 3 times. The reaction was stirred for 16 hours and then filtered through Celite, washing with EtOAc and the filtrate was concentrated in vacuo to afford the crude. The crude material was purified by silica gel column chromatography (0-80% EtOAc in heptane) to afford the title compound (1.2 g) as a colorless oil. [M+H]⁺ m/z 621.5.

### Intermediate 71

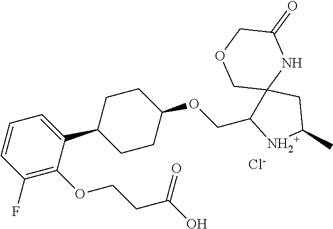 [SMILES: C[C@@H]1CC2(COCC(=O)N2)C(CO[C@H]2CC[C@@H](c3cccc(F)c3OCCC(=O)O)CC2)N1 ; [Cl-]]

### (3R)-3-methyl-7-oxo-1-({[(1s,4s)-4-[2-(2-carboxyethoxy)-3-fluorophenyl]cyclohexyl]-oxy}methyl)-9-oxa-2,6-diazaspiro[4.5]decan-2-ium chloride

4 M Hydrogen chloride in dioxane (4.8 mL, 19.3 mmol) was added to Intermediate 70 (1.20 g) at room temperature and the reaction was stirred for 2 hours. The reaction mixture was concentrated in vacuo to afford the title compound (1.0 g) as an orange solid. [M+H]⁺ m/z 463.4.

### Examples 24a and 24b

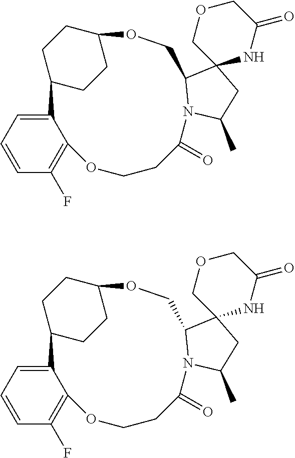 [SMILES: C[C@@H]1C[C@@]2(COCC(=O)N2)[C@@H]2CO[C@H]3CC[C@H](CC3)c3cccc(F)c3OCCC(=O)N12 ; C[C@@H]1C[C@]2(COCC(=O)N2)[C@H]2CO[C@H]3CC[C@H](CC3)c3cccc(F)c3OCCC(=O)N12]

### Example 24a: (1′s,3S,13′R,16′R,19′s)-6′-fluoro-13′-methyl-8′,18′-dioxa-12′-azaspiro[morpholine-3, 15′-tetracyclo[17.2.2.0^{2,7}0.0^{12,16}]tricosane]-2′(7′),3′,5′-triene-5,11′-dione

### Example 24b: (1′s,3R,13′R,16′S,19′s)-6′-fluoro-13′-methyl-8′,18′-dioxa-12′-azaspiro[morpholine-3, 15′-tetracyclo[17.2.2.0^{2,7}0.0^{12,16}]tricosane]-2′(7′),3′,5′-triene-5,11′-dione

To a stirred solution of HATU (908 mg, 2.39 mmol) and DIPEA (822 μL, 4.70 mmol) in acetonitrile (110 mL) was added Intermediate 71 (0.74 g) in DMF (11 mL) over 2h using a syringe pump. The resulting solution was stirred for one hour. The reaction mixture was concentrated in vacuo to afford the crude material. The crude material was diluted in water and extracted with DCM (3×5 mL) The combined organic extracts were dried (MgSO₄), filtered and concentrated in vacuo to give a solid residue. The crude material was purified by basic reverse phase column chromatography (15-45% acetonitrile in water (0.1% ammonia)) to afford the title compound (245 mg) as a yellow solid as a mixture of diastereoisomers. The mixture of diastereoisomers (245 mg) was subjected to achiral preparative purification eluting with 5-95% acetonitrile in water (0.2% ammonia), X-Bridge (100×30 mm), 5 μm, flow rate 40 mL/minute to afford the title compounds (Peak 1, 43 mg) as a white solid and (Peak 2, 26 mg) as a white solid.

### Example 24a: Peak 1

LCMS (Method A): [M+H]⁺ m/z 447.3, RT 3.15 min

¹H NMR (500 MHZ, CDCl₃) δ 7.46 (s, 1H), 6.98-6.74 (m, 3H), 5.87 (s, 1H), 5.27 (s, 0H), 4.78-4.57 (m, 1H), 4.45-3.96 (m, 5H), 3.99-3.60 (m, 4H), 3.56-3.35 (m, 1H), 3.22-3.06 (m, 1H), 2.54-2.43 (m, 1H), 2.40-1.88 (m, 7H), 1.60 (d, J=6.9 Hz, 2H), 1.50-1.41 (m, 2H), 1.38 (d, J=6.3 Hz, 1H), 1.34-1.28 (m, 1H).

### Example 24b: Peak 2

LCMS (Method A): [M+H]⁺ m/z 447.3, RT 3.27 min

¹H NMR (500 MHZ, CDCl₃) δ 6.97-6.88 (m, 2H), 6.88-6.83 (m, 1H), 6.14 (s, 1H), 4.63 (dd, J=9.7, 2.4 Hz, 1H), 4.56 (ddd, J=8.8, 4.9, 2.5 Hz, 1H), 4.46 (dq, J=9.4, 6.6 Hz, 1H), 4.39 (d, J=1.9 Hz, 1H), 4.28-4.19 (m, 2H), 4.12 (d, J=16.7 Hz, 1H), 4.06-3.98 (m, 1H), 3.79 (s, 1H), 3.62 (d, J=11.6 Hz, 1H), 3.21 (ddd, J=13.9, 11.0, 2.6 Hz, 1H), 3.15 (dd, J=9.7, 1.5 Hz, 1H), 2.67-2.55 (m, 1H), 2.52-2.39 (m, 2H), 2.26-2.15 (m, 2H), 2.09-1.97 (m, 1H), 1.85 (d, J=12.9 Hz, 1H), 1.82-1.76 (m, 1H), 1.54-1.45 (m, 2H), 1.40 (d, J=6.6 Hz, 3H), 1.37 (d, J=2.4 Hz, 1H), 1.35-1.27 (m, 1H).

Below examples were prepared following analogous procedures as described for Example 24a and 24b using the appropriate reagents

| | | | Obs. | |
|---|---|---|---|---|
| Ex. | Structure | Name | Mass | ¹H NMR |
| 25a | 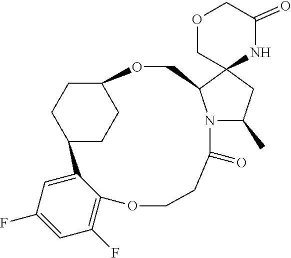 [SMILES: C[C@@H]1C[C@@]2(COCC(=O)N2)[C@@H]2CO[C@H]3CC[C@H](CC3)c3cc(F)cc(F)c3OCCC(=O)N12] | (1′s,3S,13′R,16′R, 19′s)-4′,6′- difluoro-13′- methyl-8′,18′- dioxa-12′- azaspiro[morpholine- 3,15′- tetracyclo[17.2.2. 0²,⁷.0¹²,¹⁶] tricosane]- 2′,4′,6′-triene- 5,11′-dione | LCMS (Method A): [M + H]⁺ m/z 463.4, RT 3.25 min | ¹H NMR (500 MHz, CDCl₃) δ 7.42 (s, 1H), 6.76 − 6.51 (m, 2H), 6.25 − 5.19 (m, 1H), 4.75 − 4.51 (m, 1H), 4.33 − 4.00 (m, 5H), 3.91 − 3.57 (m, 4H), 3.54 − 3.34 (m, 1H), 3.23 − 3.02 (m, 1H), 2.70 − 1.74 (m, 8H), 1.74 − 1.19 (m, 6H). |
| 25b | 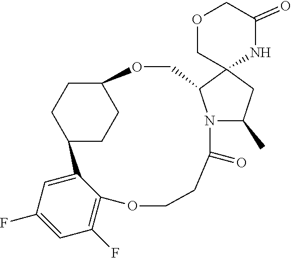 [SMILES: C[C@@H]1C[C@]2(COCC(=O)N2)[C@H]2CO[C@H]3CC[C@H](CC3)c3cc(F)cc(F)c3OCCC(=O)N12] | (1′s,3R,13′R,16′S, 19′s)-4′,6′- difluoro-13′- methyl-8′,18′- dioxa-12′- azaspiro[morpholine- 3,15′- tetracyclo[17.2.2. 0²,⁷.0¹²,¹⁶]tricosane]- 2′,4′,6′-triene- 5,11′-dione | LCMS (Method A): [M + H]⁺ m/z 463.4, RT 3.38 min | ¹H NMR (500 MHz, CDCl₃) δ 6.76 − 6.64 (m, 1H), 6.64 − 6.57 (m, 1H), 6.16 (s, 1H), 4.63 (dd, J = 9.7, 2.5 Hz, 1H), 4.54 − 4.41 (m, 2H), 4.38 (d, J = 1.9 Hz, 1H), 4.30 − 4.17 (m, 2H), 4.12 (d, J = 16.7 Hz, 1H), 4.03 (d, J = 1.0 Hz, 1H), 3.81 − 3.75 (m, 1H), 3.61 (d, J = 11.6 Hz, 1H), 3.26 − 3.11 (m, 2H), 2.58 (dd, J = 13.0, 9.6 Hz, 1H), 2.49 − 2.37 (m, 2H), 2.25 − 2.14 (m, 2H), 2.06 − 1.94 (m, 1H), 1.88 − 1.76 (m, 2H), 1.56 − 1.46 (m, 2H), 1.43 − 1.38 (m, 4H), 1.33 − 1.24 (m, 1H) |

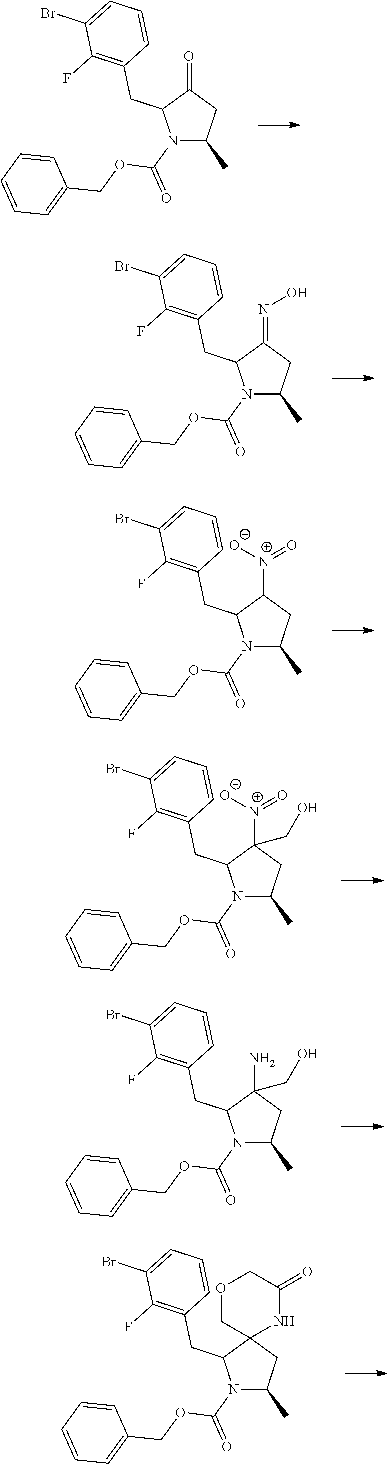

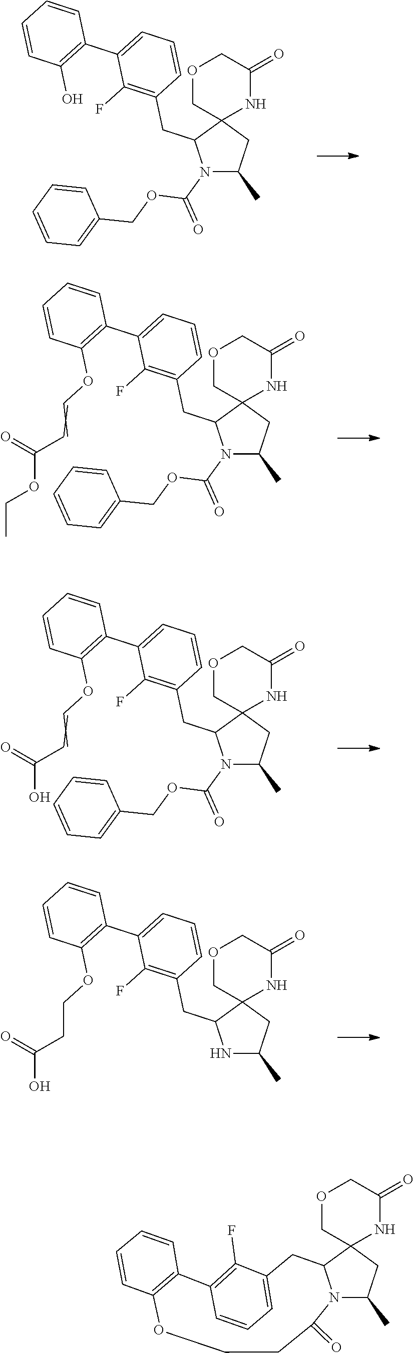

### Intermediate 72

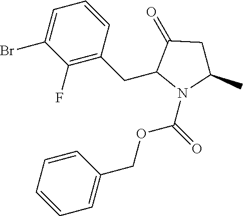 [SMILES: C[C@@H]1CC(=O)C(Cc2cccc(Br)c2F)N1C(=O)OCc1ccccc1]

### benzyl (5R)-2-[(3-bromo-2-fluorophenyl)methyl]-5-methyl-3-oxopyrrolidine-1-carboxylate

To a mixture of benzyl (2R)-2-methyl-4-oxo-pyrrolidine-1-carboxylate (52.24 g, 223.95 mmol) and toluene (200 mL) was added pyrrolidine (19.16 mL, 233.28 mmol) at room temperature. The mixture was heated under reflux for 3 hours using a Dean-stark apparatus, then the mixture was concentrated under reduced pressure. The residue was co-evaporated twice with anhydrous MeCN (2×100 mL), then dissolved in anhydrous MeCN (200 mL) under nitrogen. A solution of 1-bromo-3-(bromomethyl)-2-fluoro-benzene (50.0 g, 186.62 mmol) in anhydrous MeCN (100 mL) was added dropwise and the mixture then heated to reflux for 2 hours. The mixture was concentrated under reduced pressure, then partitioned between water (50 mL) and EtOAc (400 mL). The mixture was acidified with aqueous HCl 2N (150 mL) and stirred at room temperature for 1 hour. The biphasic mixture was separated, and the aqueous layer further extracted with EtOAc (2×100 mL). The combined organic phases were washed with water (400 mL), brine (200 mL), dried over Na₂SO₄, filtered, and concentrated under reduced pressure to give the crude material. The crude material was purified by silica gel column chromatography (5-25% EtOAc in cyclohexane) to afford the title compound (71 g) as an orange oil. [M+H]⁺ m/z 420.3, 422.3

### Intermediate 73

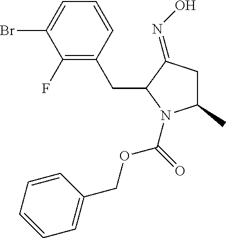 [SMILES: C[C@@H]1C/C(=N\O)C(Cc2cccc(Br)c2F)N1C(=O)OCc1ccccc1]

### Benzyl (3E,5R)-2-[(3-bromo-2-fluorophenyl)methyl]-3-(hydroxyimino)-5-methylpyrrolidine-1-carboxylate

To a solution of Intermediate 72 (71 g) and hydroxylamine hydrochloride (12.0 g, 172.69 mmol) in ethanol (300 mL) was added triethylamine (30 mL, 215.24 mmol). The solution was heated to reflux for 2 hours. The reaction mixture was cooled to room temperature and concentrated under reduced pressure. The residue was diluted with water (250 mL) and extracted with EtOAc (3×100 ml). The combined organic phases were washed with water (150 mL) and brine (150 mL), dried over Na₂SO₄, filtered, and concentrated under reduced pressure to afford the title compound (79 g) as a yellow oil. [M+H]⁺ m/z 435.3, 437.3

### Intermediate 74

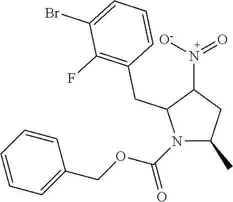

### benzyl (5R)-2-[(3-bromo-2-fluorophenyl)methyl]-5-methyl-3-nitropyrrolidine-1-carboxylate

A solution of trifluoroacetic anhydride (54.7 mL, 393.35 mmol) was added to a stirred solution of hydrogen peroxide-urea (1:1) (47.62 g, 506.23 mmol) in acetonitrile (280 mL) at 0° C. and the mixture was stirred at 0° C. for 30 mins. The resulting solution was added dropwise to a mixture of Intermediate 73 (75.0 g) and sodium hydrogen carbonate (70.55 g, 829.9 mmol) in anhydrous acetonitrile (280 mL) at 80° C. for 1 h. The reaction mixture was cooled to room temperature, quenched with saturated aqueous Na₂SO₃ (200 mL) and stirred for 10 min then extracted with EtOAc (3×150 mL). The combined organic extracts were washed with brine (250 mL), dried over Na₂SO₄, filtered, and concentrated in vacuo to afford the crude material. The crude material was purified by silica gel column chromatography (5-30% EtOAc in cyclohexane) to afford the title compound (73.6 g) as a yellow oil. [M+H]⁺ m/z 451.3, 453.3.

### Intermediate 75

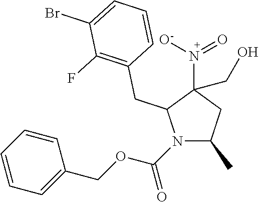

### benzyl (5R)-2-[(3-bromo-2-fluorophenyl)methyl]-3-(hydroxymethyl)-5-methyl-3-nitropyrrolidine-1-carboxylate

Formaldehyde (37% in water, 73.0 mL, 980.4 mmol) was added to Intermediate 74 (73.6 g) and triethylamine (25 mL, 179.37 mmol) in THF (280 mL) at room temperature. The solution was heated to 70° C. for 3 h. After cooling the reaction mixture was diluted with water (400 mL) and extracted with EtOAc (3×200 mL). The combined organic extracts were washed with saturated aqueous NH₄Cl (300 mL), water (200 mL) and brine (100 mL), dried (Na₂SO₄), filtered and concentrated in vacuo to afford the title compound (44 g) as a yellow oil. [M+H]⁺ m/z 481.3, 483.3

### Intermediate 76

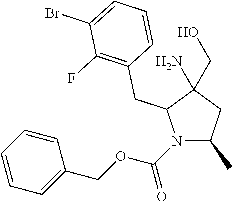 [SMILES: C[C@@H]1CC(N)(CO)C(Cc2cccc(Br)c2F)N1C(=O)OCc1ccccc1]

### benzyl (5R)-3-amino-2-[(3-bromo-2-fluorophenyl)methyl]-3-(hydroxymethyl)-5-methylpyrrolidine-1-carboxylate

A suspension of Intermediate 75 (44.0 g) and zinc (28.09 g, 429.66 mmol) in acetic acid (50 mL) and ethanol (140 mL) was stirred for 1 hour at room temperature. The mixture was then heated to 60° C. for 1 hour. The reaction mixture was cooled to room temperature and filtered through a pad of Celite, washing with methanol. The filtrate was diluted with toluene (100 mL) and concentrated under reduced pressure. The residue was partitioned between EtOAc and saturated aqueous K₂CO₃. The obtained white slurry was filtered over Celite and washed with EtOAc. The filtrate was separated, and the aqueous phase extracted with EtOAc. The combined organics were washed with brine, dried over Na₂SO₄, filtered and concentrated under reduced pressure to afford the title compound (40 g) as a yellow oil. [M+H]⁺ m/z 451.3, 453.3.

### Intermediate 77

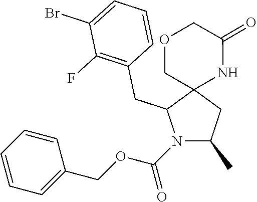 [SMILES: C[C@@H]1CC2(COCC(=O)N2)C(Cc2cccc(Br)c2F)N1C(=O)OCc1ccccc1]

### Benzyl (3R)-1-[(3-bromo-2-fluorophenyl)methyl]-3-methyl-7-oxo-9-oxa-2,6-diazaspiro[4.5]decane-2-carboxylate

To a solution of Intermediate 76 (9.2 g) in THF (45 mL) at 0° C. was added dipotassium carbonate (8.45 g, 61.15 mmol) then water (45 mL). To this mixture chloroacetyl chloride (2.27 mL, 28.54 mmol) was added dropwise at 0° C. The reaction was stirred for 1 h at 0° C. The mixture was quenched with aqueous saturated NaHCO₃ (100 mL) and extracted with EtOAc (3×100 mL). The combined organic extracts were washed with brine (40 mL), dried (Na₂SO₄), filtered and concentrated to give an oily residue. The intermediate was dissolved in DCM (45 mL) and IPA (45 mL), cooled to 0° C. potassium 2-methylpropan-2-olate (9.15 g, 81.54 mmol) was added and the reaction was stirred at 0° C. for 30 minutes. The mixture was poured onto aqueous saturated NaHCO₃ (100 ml) then concentrated to remove the DCM and IPA. The aqueous suspension was extraction with EtOAc (2×100 mL), the combined organic extracts were washed with brine (50 mL), dried (Na₂SO₄), filtered and concentrated to give the title compound (9.6 g) as a colorless oil. [M+H]⁺ m/z 491.3, 493.3.

### Intermediate 78

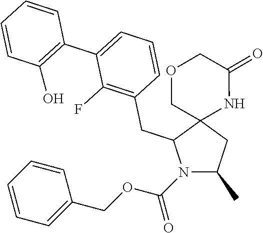 [SMILES: C[C@@H]1CC2(COCC(=O)N2)C(Cc2cccc(-c3ccccc3O)c2F)N1C(=O)OCc1ccccc1]

### Benzyl (3R)-1-({2-fluoro-2′-hydroxy-[1,1′-biphenyl]-3-yl}methyl)-3-methyl-7-oxo-9-oxa-2,6-diazaspiro[4.5]decane-2-carboxylate

XPHOS Pd G3 (258.4 mg, 0.310 mmol), Intermediate 77 (1.5 g), 1M aq. tripotassium phosphate (12.21 ml, 12.21 mmol), THF (30 mL) and 2M aqueous solution of 2-(4,4,5,5-tertamethyl-1,3,2-dioxaborolan-2-yl)phenol (767.8 μL, 3.66 mmol) were added to a microwave vial which was purged with nitrogen for 10 minutes. The reaction mixture was heated to 70° C. for 18 hours. The mixture was allowed to cool to room temperature before being filtered through Celite washing with EtOAc. The mixture was washed with water (20 mL), brine (20 mL), dried over Na₂SO₄, filtered, and concentrated under reduced pressure to give the crude material. The crude material was purified by silica gel column chromatography (0-100% EtOAc in DCM) to afford the title compound (1.16 g) as a white solid. [M+H]⁺ m/z 505.2.

### Intermediate 79

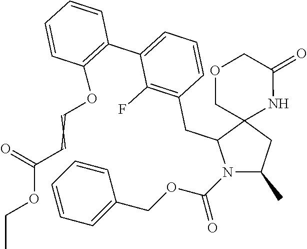 [SMILES: CCOC(=O)C=COc1ccccc1-c1cccc(CC2N(C(=O)OCc3ccccc3)[C@H](C)CC23COCC(=O)N3)c1F]

### Benzyl (3R)-1-({2′-[(3-ethoxy-3-oxoprop-1-en-1-yl)oxy]-2-fluoro-[1,1′-biphenyl]-3-yl}methyl)-3-methyl-7-oxo-9-oxa-2,6-diazaspiro[4.5]decane-2-carboxylate

To a solution of ethyl prop-2-ynoate (33.2 μL, 0.330 mmol) and 1,4-diazabicyclo[2.2.2]octane (13 mg, 0.120 mmol) in THF (7.4 mL) was added Intermediate 78 (150 mg) in THF (2.5 mL) at 0° C. under nitrogen atmosphere and the solution was stirred at room temperature for 2 hours. The reaction mixture was quenched with water (10 mL) and extracted with EtOAc (2×15 mL). The organic phase was washed with brine, dried over Na₂SO₄, and concentrated in vacuo to afford the title compound (160 mg) as a yellow solid. [M+H]⁺ m/z 603.3

### Intermediate 80

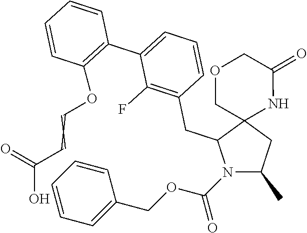 [SMILES: C[C@@H]1CC2(COCC(=O)N2)C(Cc2cccc(-c3ccccc3OC=CC(=O)O)c2F)N1C(=O)OCc1ccccc1]

### 3-[(3′-{[(3R)-2-[(benzyloxy)carbonyl]-3-methyl-7-oxo-9-oxa-2,6-diazaspiro[4.5]decan-1-yl]methyl}-2′-fluoro-[1,1′-biphenyl]-2-yl)oxy]prop-2-enoic acid

To a solution of Intermediate 79 (153 mg) in 2-Propanol (3.3 mL) and water (3.3 mL), aqueous 2 M lithium hydroxide (54.55 mg, 1.27 mmol) was added at room temperature and mixture was stirred at room temperature for 16 hours. The mixture was diluted with water (5 mL) and neutralized to pH 4 with aqueous 2M HCL then extracted with EtOAc (3×10 mL). The combined organic extracts were dried over Na₂SO₄ and concentrated in vacuo to afford the title (152 mg) as an off white solid. [M+H]⁺ m/z: 575.2

### Intermediate 81

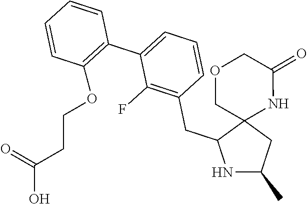 [SMILES: C[C@@H]1CC2(COCC(=O)N2)C(Cc2cccc(-c3ccccc3OCCC(=O)O)c2F)N1]

### 3-[(2′-fluoro-3′-{[(3R)-3-methyl-7-oxo-9-oxa-2,6-diazaspiro[4.5]decan-1-yl]methyl}-[1,1′-biphenyl]-2-yl)oxy]propanoic acid

Intermediate 80 (128 mg) was dissolved in methanol (5 mL) and the atmosphere was evacuated and backfilled with nitrogen 3 times. Palladium on carbon (23.71 mg, 0.020 mmol) was added and the atmosphere was evacuated and backfilled with hydrogen 3 times. The reaction was stirred for 18 hours and then filtered through Celite, washing with methanol, and concentrated in vacuo to afford the title compound (91 mg) as a white solid. [M+H]⁺ m/z 443.3

### Example 26

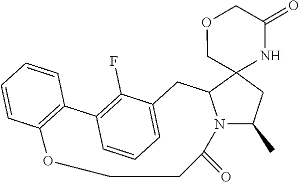 [SMILES: C[C@@H]1CC2(COCC(=O)N2)C2Cc3cccc(c3F)-c3ccccc3OCCC(=O)N21]

### (13′R)-22′-fluoro-13′-methyl-8′-oxa-12′-azaspiro[morpholine-3,15′-tetracyclo[16.3.1.0^{2,7}0.0^{12,16}]docosane]-1′(22′),2′(7′),3′,5′,18′,20′-hexaene-5,11′-dione

To a stirring solution of HATU (53.6 mg, 0.140 mmol) and DIPEA (32.74 μL,0.190 mmol) in MeCN (200 mL) under nitrogen, a solution of Intermediate 81 (77 mg) in DMA (8 mL) was added dropwise at 25-C by the aid of a syringe pump over a period of 2 h. After additional 30 min the reaction mixture was concentrated under reduced pressure. The crude mixture was taken up with diethyl ether, then washed with water: brine 1:1 (x3). The water phase was back-extracted with diethyl ether (x2). Then the combined organic extracts were filtered through a phase separator and concentrated under reduced pressure to afford crude material, which was purified by reverse column chromatography, (0-55% MeCN in Water) to afford the crude residue, which was further purified using a silica gel column chromatography (0-5% Methanol in DCM) to afford the crude product. The crude product was purified by preparative HPLC: MDAP Waters with mass spectrometry detection (MS: ZQ2000). Column: CSH C18 (30×100 mm, 3-m). Conditions: [A1: Waters+0.1% HCOOH]; [B1: MeCN]. Gradient: from 34.0% B1 to 35.0% B1 in 10 min (flow: 40.00 mL/min). Detection: UV/V is detection range 210 nm to 350 nm MS (ES+/ES−) Scan range 100 to 1000 AMU to afford the title compound (1.8 mg) as white foam.

LCMS (Method C): [M+H]⁺ m/z 425.3, RT 0.84 min

¹H NMR (400 MHZ, CDCl₃) δ 7.28-7.47 (m, 3H), 7.10-7.24 (m, 4H), 6.21 (s, 1H), 4.72 (dd, J=12.5, 3.1 Hz, 1H), 4.26-4.35 (m, 1H), 4.12-4.23 (m, 2H), 3.81-4.02 (m, 1H), 3.59-3.76 (m, 2H), 3.49 (d, J=11.6 Hz, 1H), 2.79-3.05 (m, 3H), 2.52-2.65 (m, 1H), 2.07 (dt, J=13.0, 3.1 Hz, 1H), 1.95 (dd, J=13.7, 8.7 Hz, 1H), 1.72 (d, J=6.2 Hz, 3H)

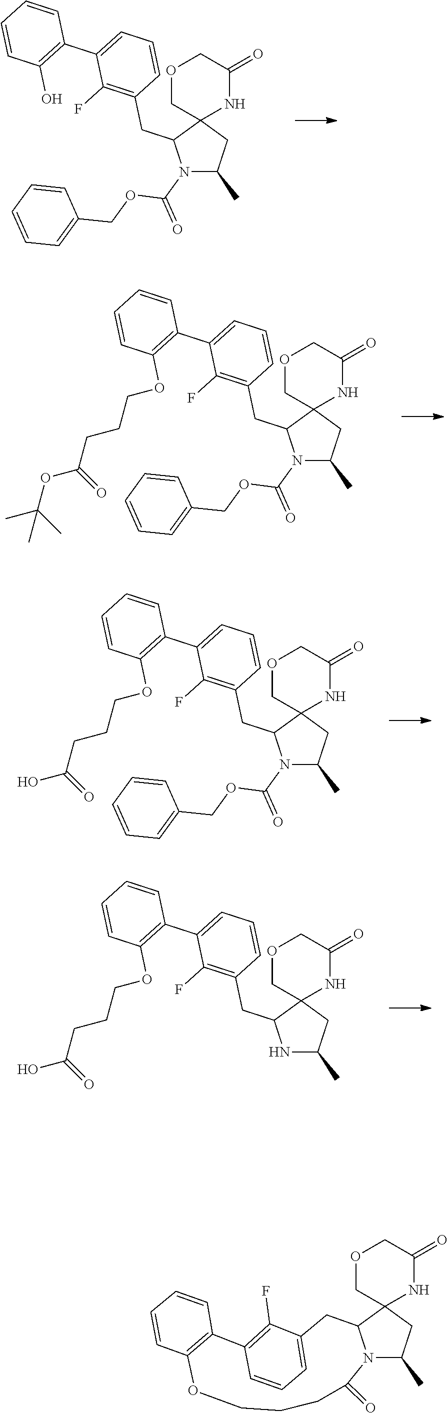

### Intermediate 82

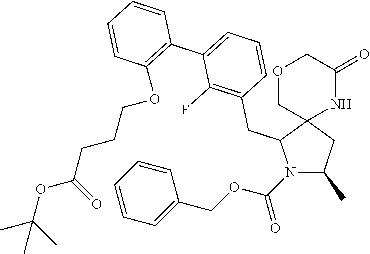 [SMILES: C[C@@H]1CC2(COCC(=O)N2)C(Cc2cccc(-c3ccccc3OCCCC(=O)OC(C)(C)C)c2F)N1C(=O)OCc1ccccc1]

### benzyl (3R)-1-({2′-[4-(tert-butoxy)-4-oxobutoxy]-2-fluoro-[1,1′-biphenyl]-3-yl}methyl)-3-methyl-7-oxo-9-oxa-2,6-diazaspiro[4.5]decane-2-carboxylate

To a solution of Intermediate 78 (150 mg), 4-hydroxy-butyric-acid-tert-butyl ester (73.2 μL, 0.450 mmol) and triphenylphosphine (116.97 mg, 0.450 mmol) in THF (3.0 mL) at 0-C under nitrogen was added Diisopropylazodicarboxylate (87.55 μL, 0.450 mmol). The ice batch was removed and the reaction mixture was heated to 70° C. overnight. Then the crude mixture was allowed to cool down, diluted with EtOAc and quenched with water. Then the water phase was back extracted with EtOAc (×2) and the combined organic extracts were filtered through a phase separator and concentrated under reduced pressure. The crude material was purified by reverse phase column chromatography (0-100% MeCN in water) to afford the title compound (143 mg) benzyl as white solid. [M+H]⁺ m/z 647.3

### Intermediate 83

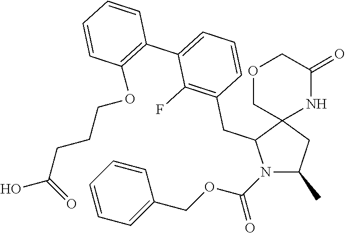 [SMILES: C[C@@H]1CC2(COCC(=O)N2)C(Cc2cccc(-c3ccccc3OCCCC(=O)O)c2F)N1C(=O)OCc1ccccc1]

### 4-[(3′-{[(3R)-2-[(benzyloxy)carbonyl]-3-methyl-7-oxo-9-oxa-2,6-diazaspiro[4.5]decan-1-yl]methyl}-2′-fluoro-[1,1′-biphenyl]-2-yl)oxy]butanoic acid

To a stirring solution Intermediate 82 (143 mg) in DCM (2.211 mL) at 25° C., was added Trifluoroacetic acid (851.72 μL, 11.06 mmol). The mixture was stirred for 2 h, then the reaction was quenched with water and diluted with DCM. The organic phase was washed with water (x2), filtered through phase separator, diluted with 10 mL of toluene and then concentrated under reduced pressure to give the title compound (150 mg) as pale yellow solid. [M+H]⁺ m/z 591.2

### Intermediate 84

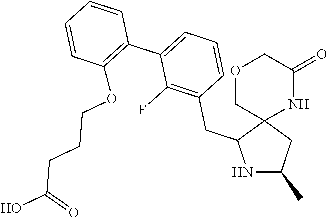 [SMILES: C[C@@H]1CC2(COCC(=O)N2)C(Cc2cccc(-c3ccccc3OCCCC(=O)O)c2F)N1]

### 4-[(2′-fluoro-3′-{[(3R)-3-methyl-7-oxo-9-oxa-2,6-diazaspiro[4.5]decan-1-yl]methyl}-[1,1′-biphenyl]-2-yl)oxy]butanoic acid

To a stirring solution of Intermediate 83 (162.5 mg) in ethanol (8.07 mL), Pd/C 10% wt. (25.77 mg, 0.020 mmol) was added and then the reaction mixture was stirred under 1 atm. of molecular hydrogen (0.49 mg, 0.240 mmol) overnight at 25° C. The reaction was filtered through a cellulose paper under vacuum washing with methanol and the filtrate was concentrated under reduced pressure to afford the title compound (113 mg) as white solid. [M+H]⁺ m/z 457.3

### Example 27

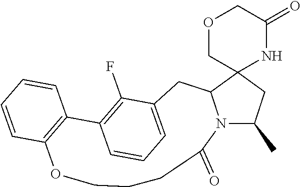 [SMILES: C[C@@H]1CC2(COCC(=O)N2)C2Cc3cccc(c3F)-c3ccccc3OCCCC(=O)N21]

### (14′R)-23′-fluoro-14′-methyl-8′-oxa-13′-azaspiro[morpholine-3,16′-tetracyclo[17.3.1.0^{2,7}0.0^{13,17}]tricosane]-1′(23′),2′(7′),3′,5′,19′,21′-hexaene-5, 12′-dione

To a stirring solution of HATU (127.43 mg, 0.340 mmol) and DIPEA (77.83 μL, 0.450 mmol) in MeCN (89.16 mL) under nitrogen, a solution of Intermediate 84 (102 mg) in DMA (8 mL) was added dropwise at 25° C. over a period of 2 h with the aid of a syringe pump. After additional 30 min the reaction mixture was concentrated under reduced pressure. The crude mixture was taken up with ethyl acetate, then washed with water (x3). The water phase was back extracted with diethyl ether (x2) then the combined organic extracts were filtered through a phase separator and concentrated under reduced pressure to afford crude product which was purified by reverse phase column chromatography (0-40% MeCN in water) to afford the title compound (83.2 mg) as white foam.

LCMS (Method C): [M+H]⁺ m/z 439.3, RT 0.92 min

¹H NMR (500 MHZ, CDCl₃) δ 7.23-7.41 (m, 2H), 7.02-7.26 (m, 4H), 6.83-7.02 (m, 1 H), 6.36-6.78 (m, 1H), 4.15-4.81 (m, 1H), 4.14-4.38 (m, 2H), 3.81-4.21 (m, 1H), 3.66-4.16 (m, 2H), 3.29-3.67 (m, 2H), 2.74-3.11 (m, 2H), 1.63-2.70 (m, 6H), 1.37-1.61 (m, 3H)

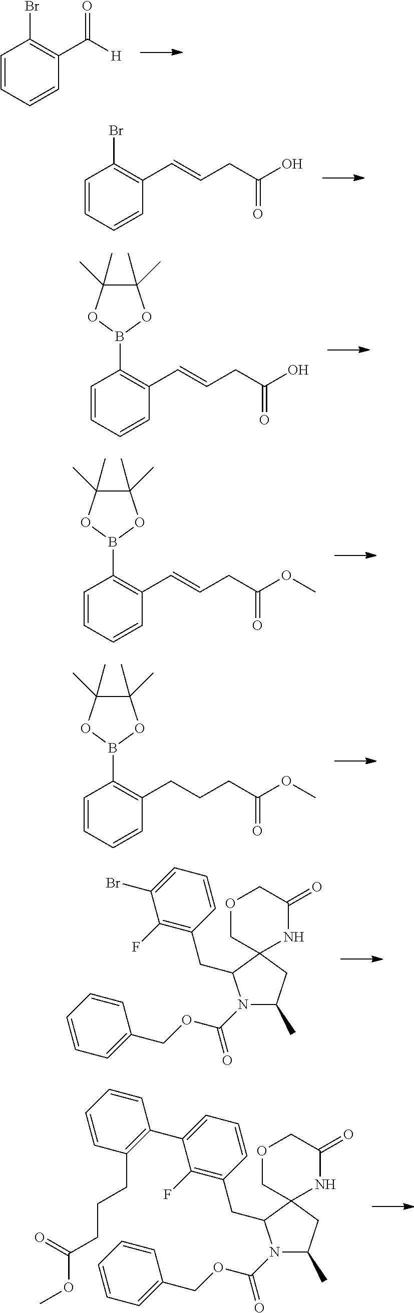

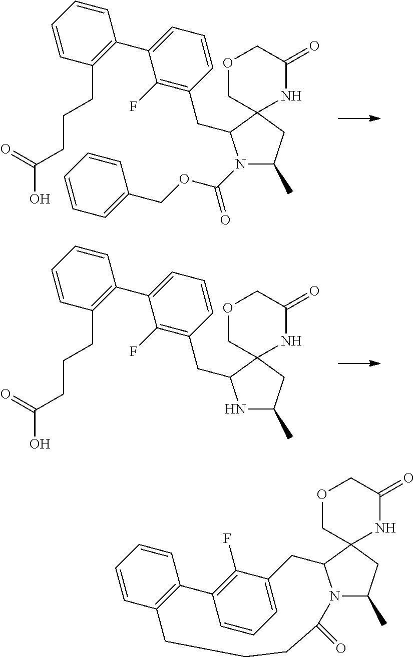

### Intermediate 85

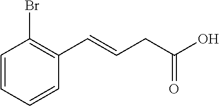 [SMILES: O=C(O)C/C=C/c1ccccc1Br]

### (3E)-4-(2-bromophenyl)but-3-enoic acid

To a suspension of 2-carboxyethyl (triphenyl)phosphonium bromide (4.94 g, 11.89 mmol) in THF (30 ml) and DMSO (10 mL) was added sodium hydride (951 mg, 23.78 mmol) at room temperature. After 10 mins, 2-bromobenzaldehyde (1.26 mL, 10.81 mmol) in THF (1.26 mL) was added dropwise. The resulting mixture was stirred at room temperature for 18 hours. The reaction mixture was then acidified to ˜pH 4 with 1M aqueous HCL and extracted with EtOAc (20 mL). The organic phase was washed with basic water (NaOH) ˜pH 8. The aqueous washing was then acidified to ˜pH 4 with 1M aqueous HCL and extracted with EtOAc (20 mL). The combined organics were washed with brine (10 mL) dried over Na₂SO₄, filtered and concentrated under reduced pressure to give the crude material. The crude material was purified by silica gel column chromatography (10% Methanol in DCM) to afford the title compound (1.45 g) as a yellow solid. [M+H]⁺ m/z241.0, 243.0

### Intermediate 86

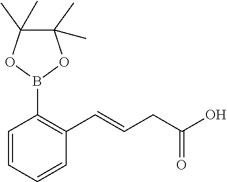 [SMILES: CC1(C)OB(c2ccccc2/C=C/CC(=O)O)OC1(C)C]

### (3E)-4-[2-(4,4,5,5-tetramethyl-1,3,2-dioxaborolan-2-yl)phenyl]but-3-enoic acid

Potassium acetate (1.33 g, 13.58 mmol), [1,1-Bis(diphenylphosphino)ferrocene]-dichloropalladium(II) (199.4 mg, 0.270 mmol), Intermediate 85 (1.31 g) and 4,4,5,5-tetramethyl-2-(4,4,5,5-tetramethyl-1,3,2-dioxaborolan-2-yl)dioxaborolane (2.07 g, 8.15 mmol), CPME (23 mL) were degassed with N₂ for 10 mins. The mixture was heated to 100° C. with stirring for 18 hours. The reaction was cooled to room temperature, filtered through a Celite pad washing with diethyl ether and concentrated under reduced pressure to afford the title compound (3.1 g) as a yellow solid. [M+H]⁺ m/z 289.2

### Intermediate 87

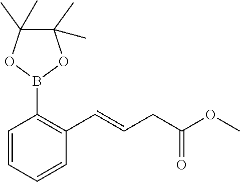

### Methyl (3E)-4-[2-(4,4,5,5-tetramethyl-1,3,2-dioxaborolan-2-yl)phenyl]but-3-enoate

To a mixture of Intermediate 86 (3.0 g) and methanol (15 mL) was added trimethylsilyldiazomethane (ca. 10% in Hexane, ca. 0.6 mol/L) (34.7 mL, 20.82 mmol) dropwise at 0° C. under an atmosphere of nitrogen. The reaction was stirred at this temperature for 1 hour. The reaction was warmed to room temperature and concentrated under reduced pressure to afford the crude material. The crude material was purified by silica gel column chromatography (0-10% EtOAc in cyclohexane) to afford the title compound (1.35 g) as a yellow solid. [M+H]⁺ m/z 303.3

### Intermediate 88

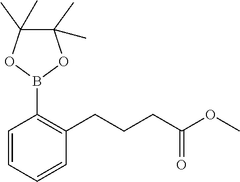

### Methyl 4-[2-(4,4,5,5-tetramethyl-1,3,2-dioxaborolan-2-yl)phenyl]butanoate

To a mixture of Intermediate 87 (1.3 g) was dissolved in methanol (30 mL) and the atmosphere was evacuated and backfilled with nitrogen 3 times. Palladium on carbon (10%, 475 mg, 0.450 mmol) was added and the atmosphere was evacuated and backfilled with hydrogen 3 times. The reaction was stirred for 1 hour and then filtered through Celite, washing with methanol, and concentrated in vacuo to afford the title compound (721 mg) as an off white solid. [M+H]⁺ m/z 305.2

### Intermediate 89

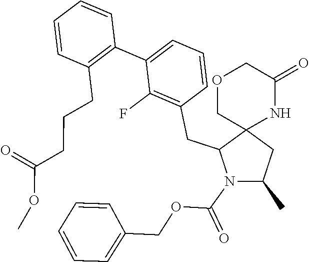 [SMILES: COC(=O)CCCc1ccccc1-c1cccc(CC2N(C(=O)OCc3ccccc3)[C@H](C)CC23COCC(=O)N3)c1F]

### benzyl (3R)-1-{[2-fluoro-2′-(4-methoxy-4-oxobutyl)-[1,1′-biphenyl]-3-yl]methyl}-3-methyl-7-oxo-9-oxa-2,6-diazaspiro[4.5]decane-2-carboxylate

XPHOS Pd G3 (137.8 mg, 0.160 mmol), Intermediate 77 (400 mg), 0.6 M aq. tripotassium phosphate (4.07 ml, 2.44 mmol), THF (10 mL) and Intermediate 88 (619 mg) were added to a microwave vial which was purged with nitrogen for 10 minutes. The reaction mixture was heated to 70° C. for 1 hour. The mixture was allowed to cool to room temperature before being filtered through Celite washing with EtOAc. The mixture was washed with water (20 mL), brine (20 mL), dried over Na₂SO₄, filtered, and concentrated under reduced pressure to give the crude material. The crude material was purified by silica gel column chromatography (0-5% Methanol in DCM) to afford the title compound (444 mg) as an orange oil. [M+H]⁺ m/z 589.3.

### Intermediate 90

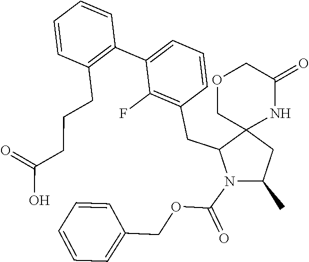 [SMILES: C[C@@H]1CC2(COCC(=O)N2)C(Cc2cccc(-c3ccccc3CCCC(=O)O)c2F)N1C(=O)OCc1ccccc1]

### 4-(3′-{[(3R)-2-[(benzyloxy)carbonyl]-3-methyl-7-oxo-9-oxa-2,6-diazaspiro[4.5]decan-1-yl]methyl}-2′-fluoro-[1,1′-biphenyl]-2-yl)butanoic acid

To a solution of Intermediate 89 (444 mg) in THF (10 mL) and water (10 mL), lithium hydroxide hydrate (124.8 mg, 2.9 mmol) was added at room temperature and mixture was stirred at room temperature for 3 hours. The mixture was diluted with water (5 mL) and neutralized to pH 4 with aqueous 1M HCL then extracted with EtOAc (3×10 mL). The combined organic extracts were dried over Na₂SO₄ and concentrated in vacuo to afford the title (468 mg) as an orange oil. [M+H]⁺ m/z: 575.4

### Intermediate 91

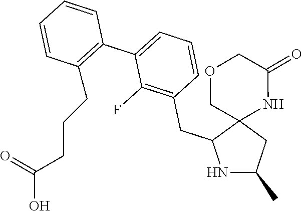 [SMILES: C[C@@H]1CC2(COCC(=O)N2)C(Cc2cccc(-c3ccccc3CCCC(=O)O)c2F)N1]

### 4-(2′-fluoro-3′-{[(3R)-3-methyl-7-oxo-9-oxa-2,6-diazaspiro[4.5]decan-1-yl]methyl}-[1,1′-biphenyl]-2-yl)butanoic acid

Intermediate 90 (468 mg) was dissolved in methanol (10 mL) and the atmosphere was evacuated and backfilled with nitrogen 3 times. Palladium on carbon (10%, 39 mg, 0.040 mmol) was added and the atmosphere was evacuated and backfilled with hydrogen 3 times. The reaction was stirred for 18 hours and then filtered through Celite, washing with methanol, and concentrated in vacuo to afford the title compound (354 mg) as a yellow oil. [M+H]⁺ m/z 441.3

### Example 28

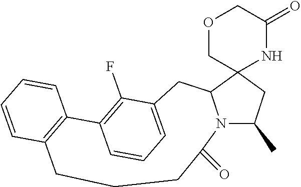 [SMILES: C[C@@H]1CC2(COCC(=O)N2)C2Cc3cccc(c3F)-c3ccccc3CCCC(=O)N21]

### (13′R)-22′-fluoro-13′-methyl-12′-azaspiro[morpholine-3,15′-tetracyclo-[16.3.1.0^{2,7}0.0^{12,16}docosane]-1′(22′),2′(7′),3′,5′,18′,20′-hexaene-5,11′-dione

To a stirring solution of HATU (307 mg, 0.810 mmol) and DIPEA (187.46 μL, 1.08 mmol) in MeCN (650 mL) under nitrogen, a solution of Intermediate 91 (354 mg) in DMA (15 mL) was added dropwise at 25° C. over a period of 2 h with the aid of a syringe pump. After additional 30 min the reaction mixture was concentrated under reduced pressure. The crude mixture was taken up with diethyl ether, then washed with water: brine 1:1 (x3). The aqueous phase was back-extracted with diethyl ether (x2) then the combined organic phases were filtered through a phase separator and concentrated under reduced pressure to afford crude material. The crude material was purified by silica gel column chromatography (0-5% methanol in DCM) to afford the crude product. The crude product was purified by preparative HPLC: MDAP Waters with mass spectrometry detection (MS: ZQ2000). Column: CSH C18 (30×100 mm, 3-m). Conditions: [A1: Waters+0.1% HCOOH]; [B1: MeCN]. Gradient: from 34.0% B1 to 35.0% B1 in 10 min (flow: 40.00 mL/min). Detection: UV/V is detection range 210 nm to 350 nm MS (ES+/ES−) Scan range 100 to 1000 AMU to afford the title compound (23 mg) as a white solid.

LCMS (Method C): [M+H]⁺ m/z 423.2, RT 0.92 min

¹H NMR (500 MHZ, CDCl₃) δ 7.24-7.40 (m, 5H), 6.96-7.21 (m, 2H), 6.28-6.59 (m, 1H), 4.57-4.84 (m, 1H), 4.07-4.36 (m, 2H), 3.74-4.00 (m, 1H), 3.66 (br d, J=11.8 Hz, 1H), 3.47 (d, J=11.8 Hz, 1H), 2.42-3.43 (m, 2H), 2.42-2.57 (m, 2H), 2.34-2.42 (m, 2H), 1.96-2.07 (m, 1H), 1.85-1.97 (m, 2H), 1.70-1.84 (m, 1H), 1.31-1.48 (m, 3H).

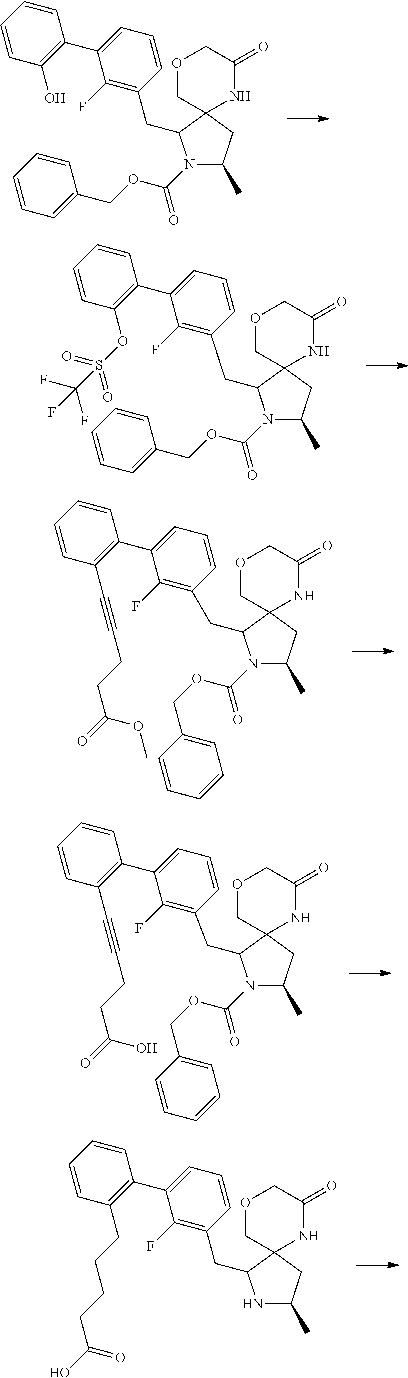

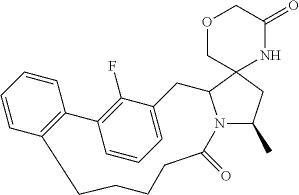 [SMILES: C[C@@H]1CC2(COCC(=O)N2)C2Cc3cccc(c3F)-c3ccccc3CCCCC(=O)N21]

### Intermediate 92

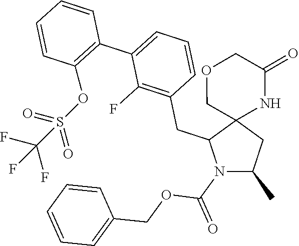 [SMILES: C[C@@H]1CC2(COCC(=O)N2)C(Cc2cccc(-c3ccccc3OS(=O)(=O)C(F)(F)F)c2F)N1C(=O)OCc1ccccc1]

### benzyl (3R)-1-{[2-fluoro-2′-(trifluoromethanesulfonyloxy)-[1,1′-biphenyl]-3-yl]methyl}-3-methyl-7-oxo-9-oxa-2,6-diazaspiro[4.5]decane-2-carboxylate

To a mixture of Intermediate 78 (300 mg) and DCM (12 mL) at −20° C., were added triethylamine (100.0 μL, 0.720 mmol) and trifluoromethanesulfonic acid trifluoromethylsulfonyl ester (100.03 μL, 0.590 mmol). The reaction was stirred at −20° C. for 1h. Additional trifluoromethanesulfonic acid trifluoromethylsulfonyl ester (17.0 μL, 0.100 mmol) was added, then the mixture was stirred overnight at room temperature. Additional triethylamine (16.57 μL, 0.120 mmol) and trifluoromethanesulfonic acid trifluoromethylsulfonyl ester (20.01 μL, 0.120 mmol) were added. Stirring was continued for 2 h then the mixture was diluted with EtOAc and washed with saturated aqueous NaHCO₃. The combined organic extracts were dried by passage through a phase separator and concentrated in vacuo to give crude material. The crude material was purified by silica gel column chromatography (0-50% EtOAc in DCM) to afford the title compound (359 mg) as a colorless oil. [M+H]⁺ m/z 637.4

### Intermediate 93

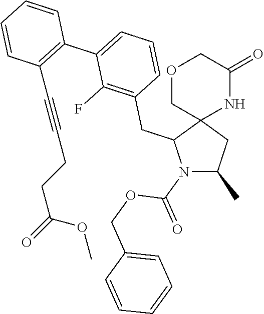 [SMILES: COC(=O)CCC#Cc1ccccc1-c1cccc(CC2N(C(=O)OCc3ccccc3)[C@H](C)CC23COCC(=O)N3)c1F]

### benzyl (3R)-1-{[2-fluoro-2′-(5-methoxy-5-oxopent-1-yn-1-yl)-[1,1′-biphenyl]-3-yl]methyl}-3-methyl-7-oxo-9-oxa-2,6-diazaspiro[4.5]decane-2-carboxylate

Dicyclohexyl-[2-[2,4,6-tri(propan-2-yl)phenyl]phenyl]phosphine (40.21 mg, 0.080 mmol), palladium dichlorobis(acetonitrile) (7.35 mg, 0.030 mmol) and cesium carbonate (549.68 mg, 1.69 mmol) were charged into a microwave vial and sealed. The vial was purged with nitrogen for 10 mins then a solution of Intermediate 92 (358 mg) in anhydrous MeCN (5.5 mL) (previously sparged with nitrogen flow for 15 min) was added, followed by the addition of methyl pent-4-ynoate (126.11 mg, 1.12 mmol). The reaction mixture was purged for a further 5 mins then heated to 80° C. for 2 hours. The mixture was cooled to room temperature and filtered through a Celite pad, then the filtrate was concentrated under reduced pressure. The crude was dissolved in EtOAc and washed with water and brine. The organic phase was filtered through a phase separator and concentrated under reduced pressure to afford crude material. The crude material was purified by silica gel column chromatography (0-100% EtOAc in DCM) to afford the title compound (274 mg) as a pale-yellow foam. [M+H]⁺ m/z 599.12

### Intermediate 94

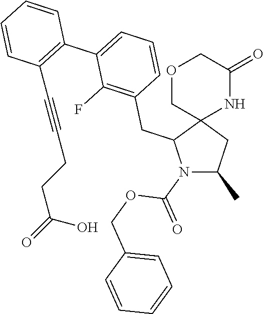 [SMILES: C[C@@H]1CC2(COCC(=O)N2)C(Cc2cccc(-c3ccccc3C#CCCC(=O)O)c2F)N1C(=O)OCc1ccccc1]

### 5-(3′-{[(3R)-2-[(benzyloxy)carbonyl]-3-methyl-7-oxo-9-oxa-2,6-diazaspiro[4.5]decan-1-yl]methyl}-2′-fluoro-[1,1′-biphenyl]-2-yl)pent-4-ynoic acid

To a stirred solution of Intermediate 93 (274 mg) in THF (6 mL) and water (6 mL) at 25° C., lithium hydroxide hydrate (98.33 mg, 2.29 mmol) was added as a solid in one portion. The reaction mixture was stirred for 20 min, then concentrated under reduced pressure to remove THF. The pH of the aqueous phase was adjusted to 2-3 with a 0.1M aq. HCl solution and then the aqueous phase was extracted with EtOAc (×3). The combined organic phases were filtered through a phase separator, concentrated under reduced pressure and triturated with diethyl ether to afford the title compound (264 mg) as a pale-yellow foam. [M+H]⁺ m/z 505.3

### Intermediate 95

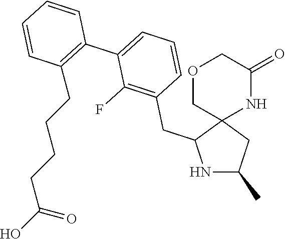 [SMILES: C[C@@H]1CC2(COCC(=O)N2)C(Cc2cccc(-c3ccccc3CCCCC(=O)O)c2F)N1]

### 5-(2′-fluoro-3′-{[(3R)-3-methyl-7-oxo-9-oxa-2,6-diazaspiro[4.5]decan-1-yl]methyl}-[1,1′-biphenyl]-2-yl)pentanoic acid

To a stirred solution of Intermediate 94 (264 mg) in ethanol (15.11 mL), Pd/C 10% wt. (48.06 mg, 0.050 mmol) was added and then the reaction mixture was stirred under 1 atm. of molecular hydrogen (0.91 mg, 0.450 mmol) for 24 h at 25° C. The reaction mixture was filtered through a cellulose filter paper under vacuum washing with methanol and the filtrate was concentrated under reduced pressure to afford the title compound (197 mg) as an off-white solid. [M+H]⁺ m/z 455.3

### Example 29

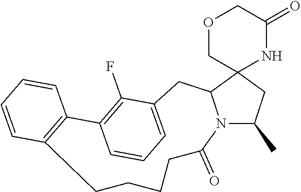 [SMILES: C[C@@H]1CC2(COCC(=O)N2)C2Cc3cccc(c3F)-c3ccccc3CCCCC(=O)N21]

### (14′R)-23′-fluoro-14′-methyl-13′-azaspiro[morpholine-3,16′tetracyclo-[17.3.1.0^{2,7}0.0^{13,17}]tricosane]-1′(23′),2′(7′),3′,5′,19′,21′-hexaene-5,12′-dione

To a stirred solution of HATU (247.2 mg, 0.650 mmol) and DIPEA (151 μL, 0.870 mmol) in MeCN (179.58 mL) under nitrogen, a solution of Intermediate 95 (197 mg) in DMA (16 mL) was added dropwise at 25° C. over a period of 2 h with the aid of a syringe pump. After additional 30 min the reaction mixture was concentrated under reduced pressure. The crude mixture was taken up with diethyl ether, then washed with water (x3). The aqueous phase was back-extracted with diethyl ether (x2) then the combined organic extracts were filtered through a phase separator and concentrated under reduced pressure to afford crude material. The crude material was purified by reverse phase column chromatography (0-40% MeCN in water) to afford the title compound (120 mg) as a pale-yellow foam.

LCMS (Method C): [M+H]⁺ m/z 437.3, RT 0.99 min

¹H NMR (500 MHZ, CDCl₃) δ 7.04-7.43 (m, 7H), 6.25-6.65 (m, 1H), 4.14-4.38 (m, 2H), 4.06-4.94 (m, 1H), 3.75-4.23 (m, 1H), 3.52-3.67 (m, 1H), 3.32-3.48 (m, 1H), 2.82-3.12 (m, 2H), 2.53-2.73 (m, 1H), 2.19-2.33 (m, 1H), 2.18-2.54 (m, 1H), 1.76-1.94 (m, 1 H), 1.43-1.57 (m, 3H), 1.11-1.43 (m, 1H), 1.01-2.08 (m, 4H), 0.76-1.38 (m, 1H)

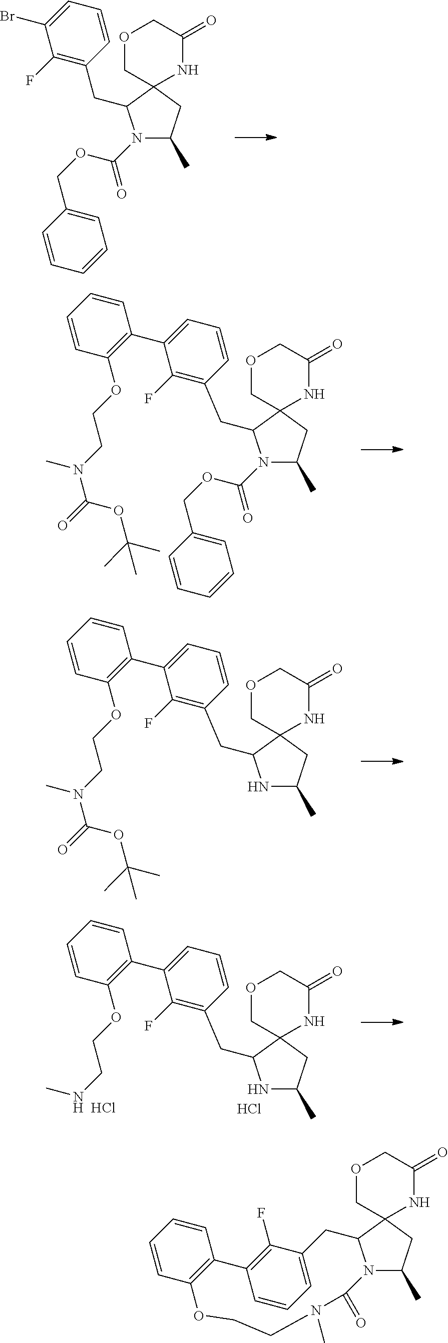

### Intermediate 96

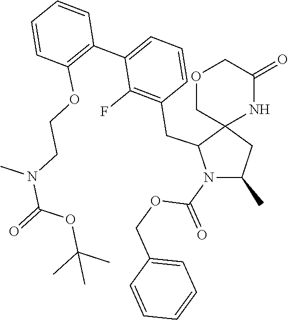 [SMILES: C[C@@H]1CC2(COCC(=O)N2)C(Cc2cccc(-c3ccccc3OCCN(C)C(=O)OC(C)(C)C)c2F)N1C(=O)OCc1ccccc1]

### Benzyl (3R)-1-{[2′-(2-{[(tert-butoxy)carbonyl](methyl) amino}ethoxy)-2-fluoro-[1,1′-biphenyl]-3-yl]methyl}-3-methyl-7-oxo-9-oxa-2,6-diazaspiro[4.5]decane-2-carboxylate

Tripotassium phosphate (610.56 μL, 1.22 mmol), [1,1-bis(diphenylphosphino)ferrocene]dichloropalladium(II) (20 mg, 0.030 mmol), Intermediate 77 (200 mg), THF (1.357 mL) and a 2M aqueous solution of tert-butyl N-methyl-N-[2-[2-(4,4,5,5-tetramethyl-1,3,2-dioxaborolan-2-yl)phenoxy]ethyl]carbamate (230.35 mg, 0.610 mmol) were added to a microwave tube. The tube was sealed, then evacuated and back-filled with nitrogen three times, then the tube was transferred into a pre-heated metal block at 70° C. and stirred for 5 h. The mixture was cooled and concentrated under reduced pressure to give the crude material. The crude material was purified by silica gel column chromatography (0-100% EtOAc in cyclohexane) to afford the title compound (198 mg) as an orange oil. [M+H]⁺ m/z 662.4

### Intermediate 97

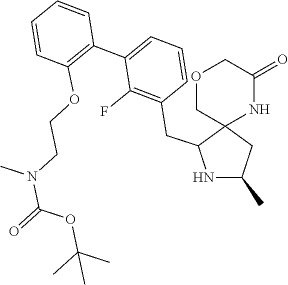 [SMILES: C[C@@H]1CC2(COCC(=O)N2)C(Cc2cccc(-c3ccccc3OCCN(C)C(=O)OC(C)(C)C)c2F)N1]

### tert-butyl N-{2-[(2′-fluoro-3′-{[(3R)-3-methyl-7-oxo-9-oxa-2,6-diazaspiro[4.5]decan-1-yl]methyl}-[1,1′-biphenyl]-2-yl)oxy]ethyl}-N-methylcarbamate

To a solution of Intermediate 96 (194 mg) in methanol (19.54 mL), palladium on carbon (10%, 106.42 mg, 0.100 mmol) was added and the reaction mixture was stirred under an atmosphere of molecular hydrogen (5 atm. pressure) for 4 h. The reaction was filtered through a Celite pad and the filtrate was concentrated under vacuum to afford the title compound (274 mg) as a yellow oil. [M+H]⁺ m/z 528.2

### Intermediate 98

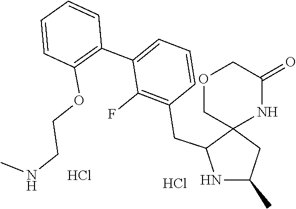 [SMILES: CNCCOc1ccccc1-c1cccc(CC2N[C@H](C)CC23COCC(=O)N3)c1F ; Cl ; Cl]

### (3R)-1-({2-fluoro-2′-[2-(methylamino)ethoxy]-[1,1′-biphenyl]-3-yl}methyl)-3-methyl-9-oxa-2,6-diazaspiro[4.5]decan-7-one dihydrochloride

Intermediate 97 (260.19 mg) was dissolved in 4M hydrogen chloride in dioxane (3.21 mL, 12.82 mmol.) The reaction mixture was stirred for 2 hours at 25° C. then concentrated under vacuum to afford the title compound (244 mg) as a white solid. [M+H]⁺ m/z 428.2

### Example 30

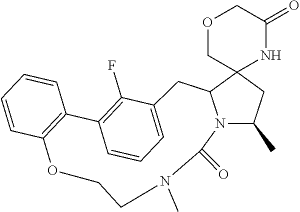 [SMILES: C[C@@H]1CC2(COCC(=O)N2)C2Cc3cccc(c3F)-c3ccccc3OCCN(C)C(=O)N21]

(14′R)-23′-fluoro-11′,14′-dimethyl-8′-oxa-11′,13′-diazaspiro[morpholine-3,16′-tetracyclo[17.3.1.0^{2,7}0.0^{13,17}]tricosane]-1′(23′),2′(7′),3′,5′,19′,21′-hexaene-5,12′-dione

Carbonic acid bis(trichloromethyl) ester (8.8 mg, 0.030 mmol) was added to a solution of Intermediate 98 (115 mg) in DCM (20 mL). The reaction mixture was stirred for 12 h at 60° C. then concentrated in vacuo to afford the crude reaction material. The crude material was purified by preparative HPLC: UPLC Waters with mass spectrometry detection (MS: SQD2). Column: CSH C18 (2.1×50 mm, 1.7-m). Conditions: [A1: Waters+0.1% HCOOH]; [B1: MeCN+0.1% HCOOH]. Gradient: from 3% B1 to 99.9% B1 in 1.4 min (flow: 0.90 mL/min). Detection: UV/Vis detection range 210 nm to 350 nm MS (ES+/ES−) Scan range 100 to 1000 AMU. to afford the title compound (3.5 mg) as a white solid.

LCMS (Method C): [M+H]⁺ m/z 454.2, RT 0.89 min

¹H NMR (400 MHZ, CDCl₃) δ 7.39-7.28 (m, 2H), 7.25-7.17 (m, 1H), 7.16-7.08 (m, 2H), 7.08-7.02 (m, 1H), 6.89 (d, J=8.2 Hz, 1H), 6.46 (br s, 1H), 4.48-4.39 (m, 1H), 4.33 (d, J=16.9 Hz, 1H), 4.24 (d, J=16.8 Hz, 1H), 4.21-4.15 (m, 1H), 4.05-3.97 (m, 1H), 3.91 (br d, J=11.5 Hz, 1H), 3.74 (br d, J=11.5 Hz, 1H), 3.74-3.68 (m, 2H), 3.66-3.56 (m, 1H), 3.19-3.01 (m, 1H), 2.87 (s, 3H), 2.71 (br d, J=13.0 Hz, 1H), 2.23 (dd, J=13.5, 8.0 Hz, 1H), 1.77 (dd, J=13.6, 3.9 Hz, 1H), 1.49 (br d, J=6.7 Hz, 3H)

### Example 31

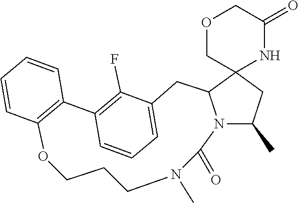 [SMILES: C[C@@H]1CC2(COCC(=O)N2)C2Cc3cccc(c3F)-c3ccccc3OCCCN(C)C(=O)N21]

### (15′R)-24′-fluoro-12′, 15′-dimethyl-8′-oxa-12′, 14′-diazaspiro[morpholine-3,17′-tetracyclo[18.3.1.0^{2,7}0.0^{14,18}]tetracosane]-1′(24′),2′(7′),3′,5′,20′,22′-hexaene-5,13′-dione

Example 31 was prepared using Intermediate 77 and tert-butyl N-methyl-N-{3-[2-(4,4,5,5-tetramethyl-1,3,2-dioxaborolan-2-yl)phenoxy]propyl}carbamate following a similar procedure as described for example 30 to afford the title compound (13.1 mg) as a pink solid.

LCMS (Method C): [M+H⁺m/z 468.2, RT 0.97 min

¹H NMR (400 MHZ, CDCl₃) δ 7.35 (t, J=8.0 Hz, 1H), 7.23 (d, J=6.2 Hz, 2H), 7.09 (dt, J=24.9, 7.3 Hz, 3H), 6.92 (d, J=8.1 Hz, 1H), 6.20 (d, J=20.1 Hz, 1H), 4.69 (d, J=10.2 Hz, 1H), 4.36-4.15 (m, 2H), 4.06-3.68 (m, 6H), 3.57 (d, J=11.6 Hz, 1H), 3.24-2.98 (m, 3H), 2.65 (s, 3H), 2.48-2.29 (m, 2H), 1.90 (s, 1H), 1.70 (dd, J=13.7, 5.3 Hz, 1H), 1.55 (bs, 3H).

### Example 32

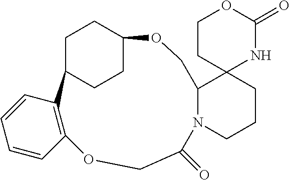 [SMILES: O=C1NC2(CCCN3C(=O)COc4ccccc4[C@H]4CC[C@H](CC4)OCC32)CCO1]

### Rel-(1′s,3S,16′R,19′s)-8′,18′-dioxa-11′-azaspiro[morpholine-3, 15′-tetracyclo-[17.2.2.0^{2,7}0.0^{11,16}]tricosane]-2′(7′),3′,5′-triene-5,10′-dione

Example 32 was prepared using commercially available starting material 1-tert-butyl 4-ethyl 3-oxopiperidine-1,4-dicarboxylate following a similar procedure as described for example 3 to afford the title compound (28 mg) as a white solid.

LCMS (Method A): [M+H]⁺ m/z 415.3, RT 2.93 min

¹H NMR (500 MHZ, CDCl₃) δ 7.16 (td, J=7.8, 1.6 Hz, 1H), 7.11-7.06 (m, 1H), 6.89 (t, J=7.4 Hz, 1H), 6.74 (d, J=8.0 Hz, 1H), 6.20 (s, 1H), 5.36 (dd, J=10.1, 3.9 Hz, 1H), 5.10 (d, J=10.6 Hz, 1H), 4.35-4.23 (m, 2H), 4.17-4.08 (m, 2H), 3.91-3.81 (m, 1H), 3.77-3.67 (m, 2H), 3.60-3.47 (m, 2H), 3.40 (d, J=11.7 Hz, 1H), 2.60 (dtd, J=28.3, 12.4, 5.5 Hz, 2H), 2.31-2.17 (m, 1H), 2.13-2.01 (m, 1H), 1.94-1.80 (m, 2H), 1.80-1.71 (m, 2H), 1.52-1.46 (m, 2H), 1.40-1.34 (m, 3H).

### Examples 32a and 32b

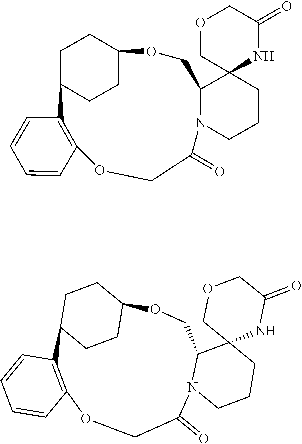 [SMILES: O=C1COC[C@@]2(CCCN3C(=O)COc4ccccc4[C@H]4CC[C@H](CC4)OC[C@H]32)N1 ; O=C1COC[C@]2(CCCN3C(=O)COc4ccccc4[C@H]4CC[C@H](CC4)OC[C@@H]32)N1]

### Example 32a: (1′s,3S,16′R,19′s)-8′,18′-dioxa-11′-azaspiro[morpholine-3,15′-tetracyclo[17.2.2.0^{2,7}0.0^{11,16}]tricosane]-2′(7′),3′,5′-triene-5,10′-dione

### Example 32b: (1′s,3R,16′S,19′s)-8′,18′-dioxa-11′-azaspiro[morpholine-3,15′-tetracyclo[17.2.2.0^{2,7}0.0^{11,16}]tricosane]-2′(7′),3′,5′-triene-5,10′-dione

Example 32 (25 mg) was subjected to chiral preparative purification using Waters 600 eluting with 60/40% v/v n-Hexane/(ethanol+0.1% isopropylamine), Chiralpak IC (25×2.0 cm), 5 μm, flow rate 17 mL/minute to afford the title compounds (Peak 1, 12.3 mg, 96.6% ee; and Peak 2, 5.6 mg, 99% ee).

### Example 32a: Peak 1 (Stereochemistry Tentatively Assigned)

LCMS (Method C): [M+H]⁺ m/z 415.2, RT 0.92 minutes.

Chiral analysis (Chiralpak IC, 25×0.46 cm, 5 μm, 60:40 n-Hexane: (ethanol+0.1% isopropylamine): RT 9.9 minutes

¹H NMR (500 MHZ, CDCl₃) δ 7.17 (td, J=7.7, 1.6 Hz, 1H), 7.10 (dd, J=7.4, 1.5 Hz, 1H), 6.93-6.88 (m, 1H), 6.75 (d, J=7.8 Hz, 1H), 5.96 (s, 1H), 5.37 (dd, J=10.0, 3.8 Hz, 1H), 5.11 (d, J=10.6 Hz, 1H), 4.33 (d, J=10.6 Hz, 1H), 4.31 (d, J=16.7 Hz, 1H), 4.15 (d, J=11.5 Hz, 1H), 4.14-4.09 (m, 1H), 3.89-3.84 (m, 1H), 3.77-3.70 (m, 2H), 3.58 (dd, J=8.5, 4.1 Hz, 1H), 3.58-3.50 (m, 1H), 3.42 (d, J=11.8 Hz, 1H), 2.70-2.60 (m, 1H), 2.62-2.53 (m, 1H), 2.30-2.20 (m, 1H), 2.09-2.03 (m, 1H), 1.92 (br d, J=13.3 Hz, 1H), 1.89-1.83 (m, 1H), 1.81-1.76 (m, 1H), 1.77-1.69 (m, 1H), 1.67-1.59 (m, 1H), 1.54-1.48 (m, 1H), 1.46-1.33 (m, 3H).

### Example 32b: Peak 2 (Stereochemistry Tentatively Assigned)

LCMS (Method C): [M+H]⁺ m/z 415.2, RT 0.92 minutes.

Chiral analysis (Chiralpak IC, 25×0.46 cm, 5 μm, 60:40 n-Hexane: (ethanol+0.1% isopropylamine): RT 14.1 minutes

¹H NMR (500 MHz, CDCl₃) δ 7.17 (td, J=7.7, 1.6 Hz, 1H), 7.10 (dd, J=7.4, 1.5 Hz, 1H), 6.93-6.88 (m, 1H), 6.75 (d, J=7.8 Hz, 1H), 5.96 (s, 1H), 5.37 (dd, J=10.0, 3.8 Hz, 1H), 5.11 (d, J=10.6 Hz, 1H), 4.33 (d, J=10.6 Hz, 1H), 4.31 (d, J=16.7 Hz, 1H), 4.15 (d, J=11.5 Hz, 1H), 4.14-4.09 (m, 1H), 3.89-3.84 (m, 1H), 3.77-3.70 (m, 2H), 3.58 (dd, J=8.5, 4.1 Hz, 1H), 3.58-3.50 (m, 1H), 3.42 (d, J=11.8 Hz, 1H), 2.70-2.60 (m, 1H), 2.62-2.53 (m, 1H), 2.30-2.20 (m, 1H), 2.09-2.03 (m, 1H), 1.92 (br d, J=13.3 Hz, 1H), 1.89-1.83 (m, 1H), 1.81-1.76 (m, 1H), 1.77-1.69 (m, 1H), 1.67-1.59 (m, 1H), 1.54-1.48 (m, 1H), 1.46-1.33 (m, 3H).

### Example 33

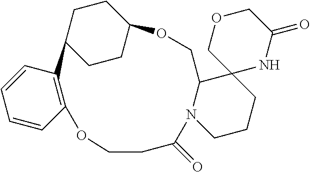 [SMILES: O=C1COCC2(CCCN3C(=O)CCOc4ccccc4[C@H]4CC[C@H](CC4)OCC32)N1]

### Rel-(1′s,3S,17′R,20's)-8′,19′-dioxa-12′-azaspiro[morpholine-3,16′-tetracyclo-[18.2.2.0^{2,7}0.0^{12,17}]tetracosane]-2′(7′),3′,5′-triene-5,11′-dione

Example 33 was prepared using commercially available starting material 1-tert-butyl 4-ethyl 3-oxopiperidine-1,4-dicarboxylate following a similar procedure as described for example 1 to afford the title compound (48 mg) as a white solid.

LCMS (Method A): [M+H]⁺ m/z 429.3, RT 2.99 minutes

¹H NMR (500 MHZ, CDCl₃) δ 8.16 (s, 1H), 7.05 (t, J=7.1 Hz, 1H), 6.94 (d, J=7.2 Hz, 1H), 6.77-6.67 (m, 2H), 4.70 (dd, J=13.8, 4.2 Hz, 1H), 4.64 (t, J=8.8 Hz, 2H), 4.23 (d, J=17.0 Hz, 1H), 4.07 (d, J=11.9 Hz, 1H), 4.02 (d, J=16.9 Hz, 1H), 3.97-3.91 (m, 1H), 3.81 (t, J=9.8 Hz, 1H), 3.75-3.68 (m, 1H), 3.57 (s, 1H), 3.43 (t, J=12.2 Hz, 1H), 3.16 (d, J=12.0 Hz, 1H), 2.70-2.57 (m, 1H), 2.58-2.49 (m, 1H), 2.44-2.31 (m, 2H), 2.19 (d, J=15.5 Hz, 1H), 1.84 (s, 1H), 1.73 (d, J=13.8 Hz, 1H), 1.66 (d, J=4.6 Hz, 2H), 1.44-1.34 (m, 2H), 1.33-1.19 (m, 4H).

### Examples 33a and 33b

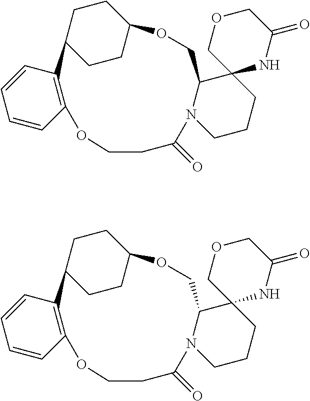 [SMILES: O=C1COC[C@@]2(CCCN3C(=O)CCOc4ccccc4[C@H]4CC[C@H](CC4)OC[C@H]32)N1 ; O=C1COC[C@]2(CCCN3C(=O)CCOc4ccccc4[C@H]4CC[C@H](CC4)OC[C@@H]32)N1]

### Example 33a: (1′s,3S, 17′R,20′s)-8′,19′-dioxa-12′-azaspiro[morpholine-3,16′-tetracyclo[18.2.2.0^{2,7}0.0^{12,17}]tetracosane]-2′(7′),3′,5′-triene-5,11′-dione

### Example 33b: (1′s,3R,17′S,20′s)-8′,19′-dioxa-12′-azaspiro[morpholine-3,16′-tetracyclo[18.2.2.0^{2,7}0.0^{12,17}]tetracosane]-2′(7′),3′,5′-triene-5,11′-dione

Example 33 (44 mg) was subjected to chiral preparative purification using Waters 600 eluting with 75/25% v/v n-Hexane/(ethanol+0.1% isopropylamine), Chiralpak OD-H (25×2.0 cm), 5 μm, flow rate 17 mL/minute to afford the title compounds (Peak 1, 10.3 mg, 100% ee; and Peak 2, 10.4 mg, 100% ee).

### Example 33a: Peak 1 (Stereochemistry Tentatively Assigned)

LCMS (Method C): [M+H]⁺ m/z 429.4, RT 0.91 minutes.

Chiral analysis (Chiralpak OD-H, 25×0.46 cm, 5 μm, 75:25 n-Hexane: (ethanol+0.1% isopropylamine): RT 7.4 minutes

¹H NMR (500 MHZ, CDCl₃) δ 7.13 (td, J=7.7, 1.6 Hz, 1H), 7.03 (br dd, J=7.4, 1.5 Hz, 1H), 7.02 (br s, 1H), 6.81 (d, J=7.7 Hz, 1H), 6.81-6.77 (m, 1H), 4.80 (br dd, J=14.1, 4.7 Hz, 1H), 4.77-4.69 (m, 2H), 4.33 (d, J=17.0 Hz, 1H), 4.16 (d, J=11.9 Hz, 1H), 4.12 (d, J=17.0 Hz, 1H), 4.02 (dt, J=7.7, 2.7 Hz, 1H), 3.88 (br t, J=9.7 Hz, 1H), 3.77 (dd, J=9.3, 2.6 Hz, 1H), 3.66 (br s, 1H), 3.56-3.46 (m, 1H), 3.26 (d, J=11.9 Hz, 1H), 2.78-2.68 (m, 1H), 2.63 (qd, J=12.6, 3.8 Hz, 1H), 2.54-2.45 (m, 1H), 2.46-2.36 (m, 1H), 2.28 (br dd, J=15.5, 1.2 Hz, 1H), 1.92 (br d, J=13.3 Hz, 1H), 1.85-1.78 (m, 1H), 1.77-1.63 (m, 3H), 1.57-1.48 (m, 1H), 1.49-1.24 (m, 4H).

### Example 33b: Peak 2 (Stereochemistry Tentatively Assigned)

LCMS (Method C): [M+H]⁺ m/z 429.5, RT 0.91 minutes.

Chiral analysis (Chiralpak OD-H, 25×0.46 cm, 5 μm, 75:25 n-Hexane: (ethanol+0.1% isopropylamine): RT 10.7 minutes

¹H NMR (500 MHZ, CDCl₃) δ 7.13 (td, J=7.7, 1.6 Hz, 1H), 7.03 (br dd, J=7.4, 1.5 Hz, 1H), 7.02 (br s, 1H), 6.81 (d, J=7.7 Hz, 1H), 6.81-6.77 (m, 1H), 4.80 (br dd, J=14.1, 4.7 Hz, 1H), 4.77-4.69 (m, 2H), 4.33 (d, J=17.0 Hz, 1H), 4.16 (d, J=11.9 Hz, 1H), 4.12 (d, J=17.0 Hz, 1H), 4.02 (dt, J=7.7, 2.7 Hz, 1H), 3.88 (br t, J=9.7 Hz, 1H), 3.77 (dd, J=9.3, 2.6 Hz, 1H), 3.66 (br s, 1H), 3.56-3.46 (m, 1H), 3.26 (d, J=11.9 Hz, 1H), 2.78-2.68 (m, 1H), 2.63 (qd, J=12.6, 3.8 Hz, 1H), 2.54-2.45 (m, 1H), 2.46-2.36 (m, 1H), 2.28 (br dd, J=15.5, 1.2 Hz, 1H), 1.92 (br d, J=13.3 Hz, 1H), 1.85-1.78 (m, 1H), 1.77-1.63 (m, 3H), 1.57-1.48 (m, 1H), 1.49-1.24 (m, 4H).

### Example 34

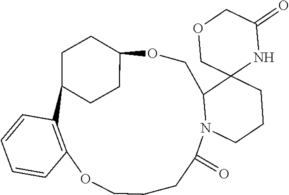 [SMILES: O=C1COCC2(CCCN3C(=O)CCCOc4ccccc4[C@H]4CC[C@H](CC4)OCC32)N1]

Rel-(1′s,3S,18′R,21′s)-8′,20′-dioxa-13′-azaspiro[morpholine-3,17′-tetracyclo[19.2.2.0^{2,7}0.0^{13,18}]pentacosane]-2′(7′),3′,5′-triene-5,12′-dione

Example 34 was prepared using commercially available starting material 1-tert-butyl 4-ethyl 3-oxopiperidine-1,4-dicarboxylate following a similar procedure as described for example 2 to afford the title compound (36 mg) as a white solid.

LCMS (Method A): [M+H]⁺ m/z 443.3, RT 3.11 minutes.

¹H NMR (500 MHZ, CDCl₃) δ 7.17-7.11 (m, 1H), 7.05 (dd, J=7.4, 1.6 Hz, 1H), 6.87 (d, J=8.1 Hz, 1H), 6.82 (t, J=7.3 Hz, 1H), 6.01 (s, 1H), 4.73-4.65 (m, 1H), 4.65-4.59 (m, 1H), 4.32 (d, J=17.0 Hz, 1H), 4.24-4.18 (m, 2H), 4.10 (d, J=17.0 Hz, 1H), 4.02 (td, J=8.8, 3.4 Hz, 1H), 3.87-3.80 (m, 1H), 3.70-3.55 (m, 3H), 3.25 (d, J=11.9 Hz, 1H), 2.71-2.61 (m, 1H), 2.49-2.32 (m, 4H), 2.26-2.16 (m, 1H), 2.08-1.97 (m, 2H), 1.85 (d, J=14.3 Hz, 1H), 1.78-1.65 (m, 2H), 1.64-1.57 (m, 1H), 1.52-1.37 (m, 5H).

### Example 35

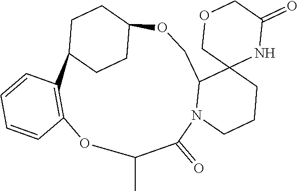 [SMILES: CC1Oc2ccccc2[C@H]2CC[C@H](CC2)OCC2N(CCCC23COCC(=O)N3)C1=O]

### Rel-(1′s,3S,16′R,19′s)-9′-methyl-8′,18′-dioxa-11′-azaspiro[morpholine-3, 15′-tetracyclo[17.2.2.0^{2,7}0.0^{11,16}]tricosane]-2′(7′),3′,5′-triene-5,10′-dione

Example 35 was prepared using commercially available starting material 1-tert-butyl 4-ethyl 3-oxopiperidine-1,4-dicarboxylate following a similar procedure as described for example 3 to afford the title compound (20 mg) as a light-yellow solid.

LCMS (Method A): [M+H]⁺ m/z 429.5, RT 2.81 minutes

¹H NMR (400 MHZ, CDCl₃) δ 7.18-7.13 (m, 1H), 7.12-7.08 (m, 1H), 6.95 (s, 1H), 6.90-6.83 (m, 1H), 6.81-6.78 (m, 1H), 5.52 (dd, J=11.4, 3.8 Hz, 1H), 5.26 (q, J=6.0 Hz, 1H), 4.28 (d, J=16.9 Hz, 1H), 4.15 (d, J=11.7 Hz, 1H), 4.08 (d, J=16.9 Hz, 1H), 3.88 (dd, J=11.5, 8.5 Hz, 1H), 3.79-3.60 (m, 2H), 3.52-3.42 (m, 2H), 3.41-3.33 (m, 2H), 2.73-2.47 (m, 2H), 2.38-2.18 (m, 1H), 2.12-2.06 (m, 1H), 1.92-1.84 (m, 1H), 1.80-1.75 (m, 1H), 1.74-1.62 (m, 3H), 1.52 (d, J=6.0 Hz, 3H), 1.43-1.32 (m, 3H).

### Examples 35a and 35b

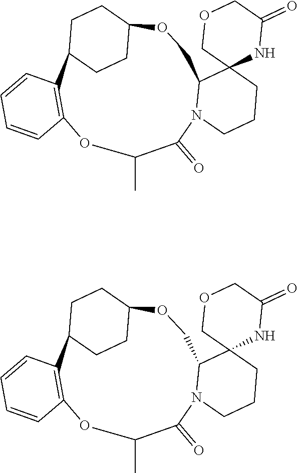 [SMILES: CC1Oc2ccccc2[C@H]2CC[C@H](CC2)OC[C@@H]2N(CCC[C@@]23COCC(=O)N3)C1=O ; CC1Oc2ccccc2[C@H]2CC[C@H](CC2)OC[C@H]2N(CCC[C@]23COCC(=O)N3)C1=O]

### Example 35a: (1′s,3S,16′R,19′s)-9′-methyl-8′,18′-dioxa-11′-azaspiro[morpholine-3,15′-tetracyclo[17.2.2.0^{2,7}0.0^{11,16}]tricosane]-2′(7′),3′,5′-triene-5,10′-dione

### Example 35b: (1′s,3R,16′S,19′s)-9′-methyl-8′,18′-dioxa-11′-azaspiro[morpholine-3,15′-tetracyclo[17.2.2.0^{2,7}0.0^{11,16}]tricosane]-2′(7′),3′,5′-triene-5,10′-dione

Example 35 (15.4 mg) was subjected to chiral preparative purification using Waters 600 eluting with 65/35% v/v n-Hexane/(ethanol/methanol 1:1+0.1% isopropylamine), Chiralpak AD-H (25×2.0 cm), 5 μm, flow rate 17 mL/minute to afford the title compounds (Peak 1, 3.32 mg, 100% ee; and Peak 2, 4.06 mg, 100% ee).

### Example 35a: Peak 1 (Stereochemistry Tentatively Assigned)

LCMS (Method C): [M+H]⁺ m/z 429.3, RT 0.95 minutes.

Chiral analysis (Chiralpak AD-H, 25×0.46 cm, 5 μm, 65:35 n-Hexane: (ethanol/methanol 1:1+0.1% isopropylamine)): RT 4.3 minutes

¹H NMR (500 MHZ, CDCl₃) δ 7.16 (td, J=7.7, 1.7 Hz, 1H), 7.10 (dd, J=7.4, 1.6 Hz, 1H), 6.87 (td, J=7.4, 1.0 Hz, 1H), 6.80 (d, J=8.1 Hz, 1H), 5.98 (br s, 1H), 5.51 (dd, J=11.3, 3.6 Hz, 1H), 5.26 (q, J=6.0 Hz, 1H), 4.30 (d, J=16.9 Hz, 1H), 4.14 (d, J=11.7 Hz, 1H), 4.11 (d, J=17.0 Hz, 1H), 3.86 (dd, J=11.4, 8.4 Hz, 1H), 3.71-3.75 (m, 1H), 3.68 (br dd, J=14.1, 3.7 Hz, 1H), 3.47 (dd, J=8.3, 3.9 Hz, 1H), 3.35-3.44 (m, 2H), 2.49-2.69 (m, 2H), 2.24-2.39 (m, 1H), 2.02-2.13 (m, 1H), 1.86-1.93 (m, 1H), 1.79-1.86 (m, 1H), 1.63-1.76 (m, 2H), 1.56 (s, 1H), 1.53 (s, 3H), 1.27-1.45 (m, 4H).

### Example 35b: Peak 2 (Stereochemistry Tentatively Assigned)

LCMS (Method C): [M+H]⁺ m/z 429.3, RT 0.95 minutes.

Chiral analysis (Chiralpak AD-H, 25×0.46 cm, 5 μm, 65:35 n-Hexane: (ethanol/methanol 1:1+0.1% isopropylamine)): RT 7.3 minutes

¹H NMR (500 MHz, CDCl₃) δ 7.16 (td, J=7.7, 1.7 Hz, 1H), 7.10 (dd, J=7.4, 1.6 Hz, 1H), 6.87 (td, J=7.4, 1.0 Hz, 1H), 6.80 (d, J=8.1 Hz, 1H), 5.98 (br s, 1H), 5.51 (dd, J=11.3, 3.6 Hz, 1H), 5.26 (q, J=6.0 Hz, 1H), 4.30 (d, J=16.9 Hz, 1H), 4.14 (d, J=11.7 Hz, 1H), 4.11 (d, J=17.0 Hz, 1H), 3.86 (dd, J=11.4, 8.4 Hz, 1H), 3.71-3.75 (m, 1H), 3.68 (br dd, J=14.1, 3.7 Hz, 1H), 3.47 (dd, J=8.3, 3.9 Hz, 1H), 3.35-3.44 (m, 2H), 2.49-2.69 (m, 2H), 2.24-2.39 (m, 1H), 2.02-2.13 (m, 1H), 1.86-1.93 (m, 1H), 1.79-1.86 (m, 1H), 1.63-1.76 (m, 2H), 1.56 (s, 1H), 1.53 (s, 3H), 1.27-1.45 (m, 4H).

### Example 36

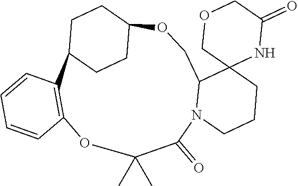 [SMILES: CC1(C)Oc2ccccc2[C@H]2CC[C@H](CC2)OCC2N(CCCC23COCC(=O)N3)C1=O]

### Rel-(1′s,3S,16′R,19′s)-9′,9′-dimethyl-8′,18′-dioxa-11′-azaspiro[morpholine-3,15′-tetracyclo[17.2.2.0^{2,7}0.0^{11,16}]tricosane]-2′(7′),3′,5′-triene-5,10′-dione

Example 36 was prepared using commercially available starting material 1-tert-butyl 4-ethyl 3-oxopiperidine-1,4-dicarboxylate following a similar procedure as described for example 3 to afford the title compound (1 mg) as a white solid.

LCMS (Method B): [M+H]⁺ m/z 443.3, RT 3.21 minutes

¹H NMR (400 MHZ, CDCl₃) δ 7.16-7.06 (m, 2H), 7.03 (dd, J=8.2, 1.3 Hz, 1H), 6.92 (td, J=7.4, 1.3 Hz, 1H), 6.09 (s, 1H), 5.49 (dd, J=10.8, 4.0 Hz, 1H), 4.52 (d, J=14.5 Hz, 1H), 4.28 (d, J=16.9 Hz, 1H), 4.16-4.05 (m, 2H), 3.84 (dd, J=10.8, 8.3 Hz, 1H), 3.74 (s, 1H), 3.54 (dd, J=8.3, 4.0 Hz, 1H), 3.49-3.34 (m, 2H), 2.66-2.41 (m, 2H), 2.23-2.04 (m, 2H), 1.97-1.82 (m, 4H), 1.78-1.64 (m, 3H), 1.53 (s, 3H), 1.45-1.32 (m, 5H).

### Example 37

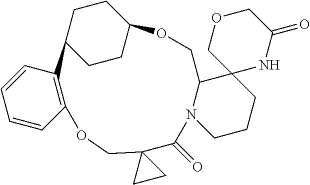 [SMILES: O=C1COCC2(CCCN3C(=O)C4(CC4)COc4ccccc4[C@H]4CC[C@H](CC4)OCC32)N1]

### Rel-(1′s,16′S,17′R,20's)-dispiro[cyclopropane-1,10′-[8,19]dioxa-[12]azatetracyclo[18.2.2.0^{2,7}0.0^{12,17}]tetracosane-16′,3″-morpholine]-2′(7′),3′,5′-triene-5″,11′-dione

Example 37 was prepared using commercially available starting material 1-tert-butyl 4-ethyl 3-oxopiperidine-1,4-dicarboxylate following a similar procedure as described for example 7 to afford the title compound (9.4 mg) as a white solid.

LCMS (Method A): [M+H]⁺ m/z 455.32, RT 3.01 minutes

¹H NMR (400 MHZ, CDCl₃) δ 7.76 (s, 1H), 7.19-7.05 (m, 2H), 6.99-6.83 (m, 2H), 5.40 (dd, J=10.1, 3.9 Hz, 1H), 4.54 (dd, J=14.2, 3.4 Hz, 1H), 4.27 (d, J=17.0 Hz, 1H), 4.17 (d, J=10.0 Hz, 1H), 4.08 (d, J=17.0 Hz, 1H), 3.96-3.87 (m, 2H), 3.83 (d, J=10.1 Hz, 1H), 3.76-3.70 (m, 1H), 3.62 (dd, J=8.8, 4.0 Hz, 1H), 3.45-3.34 (m, 1H), 3.31 (d, J=11.7 Hz, 1H), 2.53-2.34 (m, 2H), 2.31-2.19 (m, 1H), 2.18-2.12 (m, 1H), 2.02-1.97 (m, 1H), 1.91-1.69 (m, 3H), 1.57-1.39 (m, 5H), 1.34-1.22 (m, 1H), 1.14-1.07 (m, 1H), 1.07-0.96 (m, 1H), 0.87-0.79 (m, 1H).

### Examples 37a and 37b

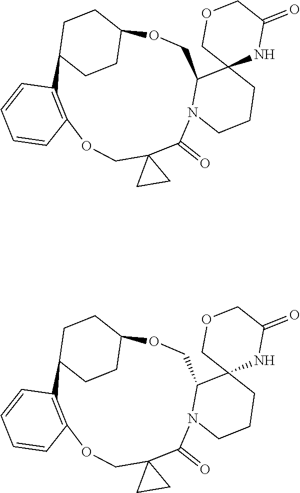 [SMILES: O=C1COC[C@@]2(CCCN3C(=O)C4(CC4)COc4ccccc4[C@H]4CC[C@H](CC4)OC[C@H]32)N1 ; O=C1COC[C@]2(CCCN3C(=O)C4(CC4)COc4ccccc4[C@H]4CC[C@H](CC4)OC[C@@H]32)N1]

### Example 37a: (1′s,16′S,17′R,20′s)-dispiro[cyclopropane-1,10′-[8,19]dioxa-[12]azatetracyclo[18.2.2.0^{2,7}0.0^{12,17}]tetracosane-16′,3″-morpholine]-2′(7′),3′,5′-triene-5″,11′-dione

### Example 37b: (1′s,16′R,17′S,20′s)-dispiro[cyclopropane-1,10′-[8,19]dioxa-[12]azatetracyclo[18.2.2.0^{2,7}0.0^{12,17}]tetracosane-16′,3″-morpholine]-2′(7′),3′,5′-triene-5″,11′-dione

Example 37 (6.1 mg) was subjected to chiral preparative purification using Waters 600 eluting with 70/30% v/v n-Hexane/(ethanol/methanol 1:1+0.1% isopropylamine), Whelk O1 (R,R) (25×2.0 cm), 5 μm, flow rate 17 mL/minute to afford the title compounds (Peak 1, 1.4 mg, 100% ee; and Peak 2, 2.6 mg, 98.7% ee).

### Example 37a: Peak 1 (Stereochemistry Tentatively Assigned)

LCMS (Method C): [M+H]⁺ m/z 455.3, RT 0.92 minutes.

Chiral analysis (Whelk O1 (R,R), 25×0.46 cm, 5 μm, 70:30 n-Hexane: (ethanol/methanol 1:1+0.1% isopropylamine)): RT 13.2 minutes

¹H NMR (500 MHZ, CDCl₃) δ 7.15 (td, J=7.8, 1.8 Hz, 1H), 7.10 (dd, J=7.8, 1.8 Hz, 1H), 6.90-6.97 (m, 2H), 5.78 (s, 1H), 5.31 (dd, J=8.9, 4.0 Hz, 1H), 4.57 (br dd, J=14.3, 3.8 Hz, 1H), 4.27 (d, J=16.7 Hz, 1H), 4.21 (d, J=10.2 Hz, 1H), 4.10 (d, J=17.0 Hz, 1H), 3.89 (d, J=11.8 Hz, 1H), 3.83 (t, J=8.8 Hz, 1H), 3.77 (d, J=10.2 Hz, 1H), 3.72 (br s, 1H), 3.68-3.72 (m, 1H), 3.44 (td, J=13.6, 3.0 Hz, 1H), 3.34 (d, J=11.8 Hz, 1H), 2.42-2.54 (m, 1H), 1.21-2.41 (m, 12H), 0.76-1.32 (m, 4H).

### Example 37b: Peak 2 (Stereochemistry Tentatively Assigned)

LCMS (Method C): [M+H]⁺ m/z 455.3, RT 0.92 minutes.

Chiral analysis (Whelk 01 (R,R), 25×0.46 cm, 5 μm, 70:30 n-Hexane: (ethanol/methanol 1:1+0.1% isopropylamine)): RT 15.3 minutes

¹H NMR (500 MHZ, CDCl₃) δ 7.15 (td, J=7.8, 1.8 Hz, 1H), 7.10 (dd, J=7.8, 1.8 Hz, 1H), 6.90-6.97 (m, 2H), 5.78 (s, 1H), 5.31 (dd, J=8.9, 4.0 Hz, 1H), 4.57 (br dd, J=14.3, 3.8 Hz, 1H), 4.27 (d, J=16.7 Hz, 1H), 4.21 (d, J=10.2 Hz, 1H), 4.10 (d, J=17.0 Hz, 1H), 3.89 (d, J=11.8 Hz, 1H), 3.83 (t, J=8.8 Hz, 1H), 3.77 (d, J=10.2 Hz, 1H), 3.72 (br s, 1H), 3.68-3.72 (m, 1H), 3.44 (td, J=13.6, 3.0 Hz, 1H), 3.34 (d, J=11.8 Hz, 1H), 2.42-2.54 (m, 1H), 1.21-2.41 (m, 12H), 0.76-1.32 (m, 4H).

### Examples 38a and 38b

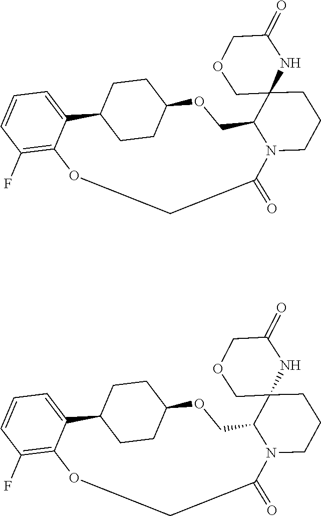 [SMILES: O=C1COC[C@@]2(CCCN3C(=O)COc4c(F)cccc4[C@H]4CC[C@H](CC4)OC[C@H]32)N1 ; O=C1COC[C@]2(CCCN3C(=O)COc4c(F)cccc4[C@H]4CC[C@H](CC4)OC[C@@H]32)N1]

### Example 38a: (1′s,3S,16′R,19′s)-6′-fluoro-8′,18′-dioxa-11′-azaspiro[morpholine-3,15′-tetracyclo[17.2.2.0^{2,7}0.0^{11,16}]tricosane]-2′(7′),3′,5′-triene-5,10′-dione

### Example 38b: (1′s,3R,16′S,19′s)-6′-fluoro-8′,18′-dioxa-11′-azaspiro[morpholine-3,15′-tetracyclo[17.2.2.0^{2,7}0.0^{11,16}]tricosane]-2′(7′),3′,5′-triene-5,10′-dione

Examples 38a and 38b were prepared using commercially available starting material 1-tert-butyl 4-ethyl 3-oxopiperidine-1,4-dicarboxylate following a similar procedure as described for examples 17a and 17b. The racemic mixture (8.6 mg) was subjected to chiral preparative purification using Waters 600 eluting with 45/55% v/v n-Hexane/ethanol+0.1% isopropylamine), Chiralcel OJ-C(25×2.0 cm), 5 μm, flow rate 17 mL/minute to afford the title compounds (Peak 1, 1.5 mg, 100% ee; and Peak 2, 1.72 mg, 100% ee).

### Example 38a: Peak 1 (Stereochemistry Tentatively Assigned)

LCMS (Method C): [M+H]⁺ m/z 433.3, RT 0.94 minutes.

Chiral analysis (Chiralcel OJ-H, 25×0.46 cm, 5 μm, 45:55 n-Hexane: ethanol+0.1% isopropylamine): RT 5.9 minutes

¹H NMR (500 MHZ, CDCl₃) δ 6.80-7.01 (m, 3H), 5.99 (br s, 1H), 5.32-5.50 (m, 2H), 4.35 (dd, J=12.5, 1.4 Hz, 1H), 4.31 (d, J=17.0 Hz, 1H), 4.13-4.17 (m, 1H), 4.11 (d, J=17.0 Hz, 1H), 3.92-4.00 (m, 1H), 3.84 (dd, J=10.0, 8.5 Hz, 1H), 3.74-3.79 (m, 1H), 3.55-3.60 (m, 1H), 3.47-3.55 (m, 1H), 3.40 (d, J=11.7 Hz, 1H), 2.63-2.73 (m, 1H), 2.53-2.64 (m, 1H), 1.20-2.16 (m, 11H).

### Example 38b: Peak 2 (Stereochemistry Tentatively Assigned)

LCMS (Method C): [M+H]⁺ m/z 433.3, RT 0.94 minutes.

Chiral analysis (Chiralcel OJ-H, 25×0.46 cm, 5 μm, 45:55 n-Hexane: ethanol+0.1% isopropylamine): RT 10.0 minutes

¹H NMR (500 MHZ, CDCl₃) δ 6.80-7.01 (m, 3H), 5.99 (br s, 1H), 5.32-5.50 (m, 2H), 4.35 (dd, J=12.5, 1.4 Hz, 1H), 4.31 (d, J=17.0 Hz, 1H), 4.13-4.17 (m, 1H), 4.11 (d, J=17.0 Hz, 1H), 3.92-4.00 (m, 1H), 3.84 (dd, J=10.0, 8.5 Hz, 1H), 3.74-3.79 (m, 1H), 3.55-3.60 (m, 1H), 3.47-3.55 (m, 1H), 3.40 (d, J=11.7 Hz, 1H), 2.63-2.73 (m, 1H), 2.53-2.64 (m, 1H), 1.20-2.16 (m, 11H).

Below examples were prepared following analogous procedures as described for Example 38a and 38b using the appropriate reagents

| | | | Obs. | |
|---|---|---|---|---|
| Ex. | Structure | Name | Mass | ¹H NMR |
| 39a | 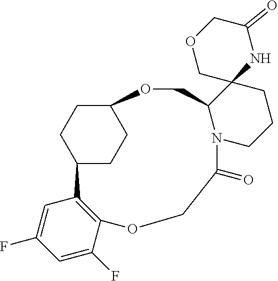 [SMILES: O=C1COC[C@@]2(CCCN3C(=O)COc4c(F)cc(F)cc4[C@H]4CC[C@H](CC4)OC[C@H]32)N1] | (1′s,3S,16′R,19′s)- 4′,6′-difluoro-8′,18′- dioxa-11′- azaspiro[morpholine- 3,15′- tetracyclo[17.2.2.0², ⁷.0¹¹,¹⁶]tricosane]- 2′(7′),3′,5′-triene- 5,10′-dione | LCMS (Method C): [M + H]⁺ m/z 451.3, RT 0.96 minutes | ¹H NMR (500 MHz, CDCl₃) δ 6.71 (ddd, J = 11.3, 8.2, 3.0 Hz, 1H), 6.62 (dt, J = 8.8, 2.2 Hz, 1H), 6.15 (s, 1H), 5.30 − 5.47 (m, 2H), 4.24 − 4.40 (m, 2H), 4.05 − 4.19 (m, 2H), 3.93 (br dd, J = 14.0, 3.6 Hz, 1H), 3.84 (dd, J = 10.3, 8.4 Hz, 1H), 3.75 (brs, 1H), 3.56 (dd, J = 8.2, 3.8 Hz, 1H), 3.44 − 3.53 (m, 1H), 3.40 (d, J = 11.8 Hz, 1H), 2.60 − 2.71 (m, 1H), 2.49 − 2.59 (m, 1H), 1.15 − 2.16 (m, 11H). |
| 39b | 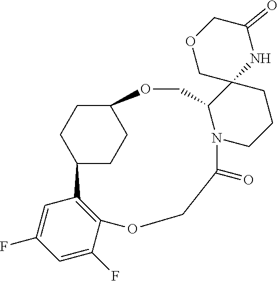 [SMILES: O=C1COC[C@]2(CCCN3C(=O)COc4c(F)cc(F)cc4[C@H]4CC[C@H](CC4)OC[C@@H]32)N1] | (1′s,3R,16′S,19′s)- 4′,6′-difluoro-8′,18′- dioxa-11′- azaspiro [morpholine- 3,15′- tetracyclo[17.2.2. 0²,⁷.0¹¹,¹⁶]tricosane]- 2′(7′),3′,5′-triene- 5,10′-dione | LCMS (Method C): [M + H]⁺ m/z 451.3, RT 0.96 minutes | ¹H NMR (500 MHz, CDCl₃) δ 6.71 (ddd, J = 11.3, 8.2, 3.0 Hz, 1H), 6.62 (dt, J = 8.8, 2.2 Hz, 1H), 6.15 (s, 1H), 5.30 − 5.47 (m, 2H), 4.24 − 4.40 (m, 2H), 4.05 − 4.19 (m, 2H), 3.93 (br dd, J = 14.0, 3.6 Hz, 1H), 3.84 (dd, J = 10.3, 8.4 Hz, 1H), 3.75 (brs, 1H), 3.56 (dd, J = 8.2, 3.8 Hz, 1H), 3.44 − 3.53 (m, 1H), 3.40 (d, J = 11.8 Hz, 1H), 2.60 − 2.71 (m, 1H), 2.49 − 2.59 (m, 1H), 1.15 − 2.16 (m, 11H). |
| 40a | 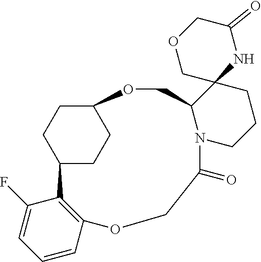 [SMILES: O=C1COC[C@@]2(CCCN3C(=O)COc4cccc(F)c4[C@H]4CC[C@H](CC4)OC[C@H]32)N1] | (1′s,3S,16′R,19′s)- 3′-fluoro-8′,18′- dioxa-11′- azaspiro[morpholine- 3,15′- tetracyclo[17.2.2. 0²,⁷.0¹¹,¹⁶]tricosane]- 2′(7′),3′,5′-triene- 5,10′-dione | LCMS (Method B): [M + H]⁺ m/z 433.3, RT 2.77 minutes | ¹H NMR (400 MHz, CDCl₃) δ 7.08 (q, J = 7.6 Hz, 1H), 6.70 (t, J = 8.9 Hz, 1H), 6.53 (d, J = 7.2 Hz, 2H), 5.37 (dd, J = 10.4, 4.0 Hz, 1H), 5.08 (d, J = 10.5 Hz, 1H), 4.36 − 4.25 (m, 2H), 4.18 − 4.03 (m, 2H), 3.92 − 3.82 (m, 1H), 3.75 − 3.66 (m, 2H), 3.60 − 3.47 (m, 2H), 3.40 (d, J = 11.7 Hz, 1H), 3.21 − 3.10 (m, 1H), 2.67 − 2.50 (m, 1H), 2.28 − 2.13 (m, 1H), 2.11 − 1.99 (m, 2H), 1.94 − 1.71 (m, 4H), 1.51 − 1.19 (m, 4H). |
| 40b | 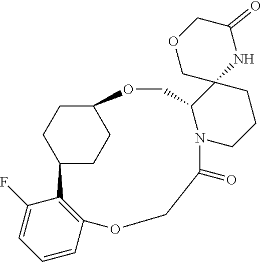 [SMILES: O=C1COC[C@]2(CCCN3C(=O)COc4cccc(F)c4[C@H]4CC[C@H](CC4)OC[C@@H]32)N1] | (1′s,3R,16′S,19′s)- 3′-fluoro-8′,18′- dioxa-11′- azaspiro[morpholine- 3,15′- tetracyclo[17.2.2. 0²,⁷.0¹¹,¹⁶]tricosane]- 2′(7′),3′,5′-triene- 5,10′-dione | LCMS (Method B): [M + H]⁺ m/z 433.3, RT 2.77 minutes | ¹H NMR (400 MHz, CDCl₃) δ 7.08 (td, J = 8.2, 6.2 Hz, 1H), 6.69 (td, J = 8.8, 4.2 Hz, 1H), 6.53 (d, J = 8.2 Hz, 1H), 6.31 (s, 1H), 5.37 (d, J = 9.1 Hz, 1H), 5.09 (d, J = 10.3 Hz, 1H), 4.36 − 4.25 (m, 2H), 4.25 − 4.03 (m, 2H), 3.91 − 3.82 (m, 1H), 3.78 − 3.65 (m, 2H), 3.59 − 3.47 (m, 2H), 3.40 (d, J = 11.2 Hz, 1H), 3.22 − 3.09 (m, 1H), 2.65 − 2.50 (m, 1H), 2.21 (qd, J = 13.0, 4.1 Hz, 1H), 2.11 − 1.98 (m, 2H), 1.96 − 1.68 (m, 4H), 1.49 − 1.28 (m, 4H). |
| 41 | 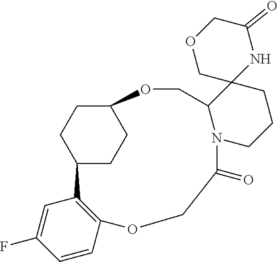 [SMILES: O=C1COCC2(CCCN3C(=O)COc4ccc(F)cc4[C@H]4CC[C@H](CC4)OCC32)N1] | Rel- (1′s,3S,16′R,19′s)- 4′-fluoro-8′,18′- dioxa-11′- azaspiro[morpholine- 3,15′- tetracyclo[17.2.2. 0²,⁷.0¹¹,¹⁶]tricosane]- 2′(7′),3′,5′-triene- 5,10′-dione | LCMS (Method B): [M + H]⁺ m/z 433.3, RT 2.85 minutes | ¹H NMR (400 MHz, CDCl₃) δ 6.90 − 6.77 (m, 2H), 6.67 (dd, J = 8.5, 4.6 Hz, 1H), 6.51 (s, 1H), 5.36 (dd, J = 10.3, 4.0 Hz, 1H), 5.08 (d, J = 10.7 Hz, 1H), 4.34 − 4.22 (m, 2H), 4.19 − 4.04 (m, 2H), 3.91 − 3.78 (m, 1H), 3.70 (d, J = 16.9 Hz, 2H), 3.59 − 3.43 (m, 2H), 3.39 (d, J = 11.7 Hz, 1H), 2.71 − 2.43 (m, 2H), 2.26 − 2.02 (m, 2H), 1.94 − 1.68 (m, 4H), 1.54 − 1.17 (m, 5H). |
| 41a | 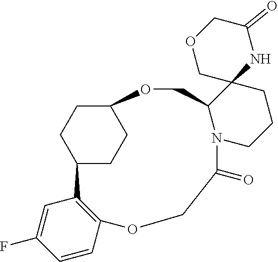 [SMILES: O=C1COC[C@@]2(CCCN3C(=O)COc4ccc(F)cc4[C@H]4CC[C@H](CC4)OC[C@H]32)N1] | (1′s,3S,16′R,19′s)- 4′-fluoro-8′,18′- dioxa-11′- azaspiro[morpholine- 3,15′- tetracyclo[17.2.2. 0²,⁷.0¹¹,¹⁶]tricosane]- 2′(7′),3′,5′-triene- 5,10′-dione | LCMS (Method C): [M + H]⁺ m/z 433.3, RT 0.92 minutes | ¹H NMR (500 MHz, CDCl₃) δ 6.96 (br s, 1H), 6.79 − 6.88 (m, 2H), 6.67 (dd, J = 8.5, 4.5 Hz, 1H), 5.38 (dd, J = 10.4, 3.9 Hz, 1H), 5.09 (d, J = 10.6 Hz, 1H), 4.26 − 4.34 (m, 2H), 4.16 (d, J = 11.7 Hz, 1H), 4.10 (d, J = 17.0 Hz, 1H), 3.88 (dd, J = 10.1, 8.9 Hz, 1H), 3.68 − 3.77 (m, 2H), 3.54 (dd, J = 8.4, 4.0 Hz, 1H), 3.47 − 3.53 (m, 1H), 3.38 (d, J = 11.7 Hz, 1H), 2.58 − 2.70 (m, 1H), 2.46 − 2.57 (m, 1H), 2.16 − 2.29 (m, 1H), 2.05 − 2.13 (m, 1H), 1.31 − 1.94 (m, 9H). |
| 41b | 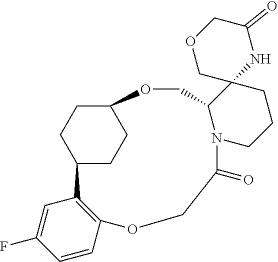 [SMILES: O=C1COC[C@]2(CCCN3C(=O)COc4ccc(F)cc4[C@H]4CC[C@H](CC4)OC[C@@H]32)N1] | (1′s,3R,16′S,19′s)- 4′-fluoro-8′,18′- dioxa-11′- azaspiro[morpholine- 3,15′- tetracyclo[17.2.2. 0²,⁷.0¹¹,¹⁶]tricosane]- 2′(7′),3′,5′-triene- 5,10′-dione | LCMS (Method C): [M + H]⁺ m/z 433.3, RT 0.92 minutes | ¹H NMR (500 MHz, CDCl₃) δ 6.96 (br s, 1H), 6.79 − 6.88 (m, 2H), 6.67 (dd, J = 8.5, 4.5 Hz, 1H), 5.38 (dd, J = 10.4, 3.9 Hz, 1H), 5.09 (d, J = 10.6 Hz, 1H), 4.26 − 4.34 (m, 2H), 4.16 (d, J = 11.7 Hz, 1H), 4.10 (d, J = 17.0 Hz, 1H), 3.88 (dd, J = 10.1, 8.9 Hz, 1H), 3.68 − 3.77 (m, 2H), 3.54 (dd, J = 8.4, 4.0 Hz, 1H), 3.47 − 3.53 (m, 1H), 3.38 (d, J = 11.7 Hz, 1H), 2.58 − 2.70 (m, 1H), 2.46 − 2.57 (m, 1H), 2.16 − 2.29 (m, 1H), 2.05 − 2.13 (m, 1H), 1.31 − 1.94 (m, 9H). |
| 42a | 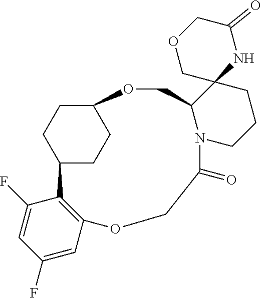 [SMILES: O=C1COC[C@@]2(CCCN3C(=O)COc4cc(F)cc(F)c4[C@H]4CC[C@H](CC4)OC[C@H]32)N1] | (1′s,3S,16′R,19′s)- 3′,5′-difluoro-8′,18′- dioxa-11′- azaspiro[morpholine- 3,15′- tetracyclo[17.2.2. 0²,⁷.0¹¹,¹⁶]tricosane]- 2′(7′),3′,5′-triene- 5,10′-dione | LCMS (Method B): [M + H]⁺ m/z 451.3, RT 2.93 minutes | ¹H NMR (400 MHz, CDCl₃) δ 6.75 (s, 1H), 6.45 (td, J = 9.4, 2.5 Hz, 1H), 6.29 (dt, J = 9.8, 2.0 Hz, 1H), 5.37 (dd, J = 10.5, 4.1 Hz, 1H), 5.04 (d, J = 10.3 Hz, 1H), 4.84 − 4.53 (m, 1H), 4.32 − 4.24 (m, 2H), 4.18 − 4.03 (m, 2H), 3.92 − 3.80 (m, 1H), 3.79 − 3.60 (m, 2H), 3.60 − 3.44 (m, 2H), 3.38 (d, J = 11.7 Hz, 1H), 3.09 (tt, J = 11.9, 5.2 Hz, 1H), 2.59 − 2.42 (m, 1H), 2.27 − 2.03 (m, 2H), 1.84 (m, 4H), 1.50 − 1.19 (m, 4H) |
| 42b | 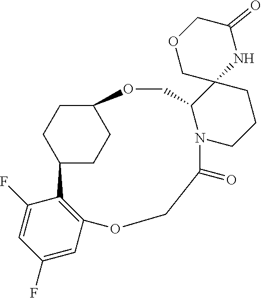 [SMILES: O=C1COC[C@]2(CCCN3C(=O)COc4cc(F)cc(F)c4[C@H]4CC[C@H](CC4)OC[C@@H]32)N1] | (1′s,3S,16′R,19′s)- 3′,5′-difluoro-8′,18′- dioxa-11′- azaspiro[morpholine- 3,15′- tetracyclo[17.2.2. 0²,⁷.0¹¹,¹⁶]tricosane]- 2′(7′),3′,5′-triene- 5,10′-dione | LCMS (Method B): [M + H]⁺ m/z 451.3, RT 2.93 minutes | ¹H NMR (400 MHz, CDCl₃) δ 6.52 6.34 (m, 2H), 6.29 (dt, J = 9.7, 2.0 Hz, 1H), 5.37 (dd, J = 10.3, 4.0 Hz, 1H), 5.04 (d, J = 10.3 Hz, 1H), 4.85 − 4.51 (m, 1H), 4.34 − 4.21 (m, 2H), 4.21 − 4.04 (m, 2H), 3.92 − 3.82 (m, 1H), 3.75 − 3.71 (m, 1H), 3.71 − 3.62 (m, 1H), 3.59 − 3.45 (m, 2H), 3.39 (d, J = 11.7 Hz, 1H), 3.10 (ddq, J = 17.9, 12.0, 5.8 Hz, 1H), 2.57 − 2.42 (m, 1H), 2.25 − 1.95 (m, 2H), 1.93 − 1.71 (m, 4H), 1.49 − 1.34 (m, 4H). |
| 43a | 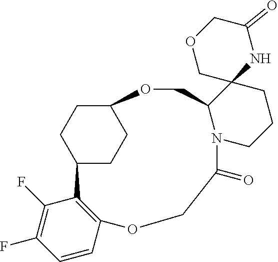 [SMILES: O=C1COC[C@@]2(CCCN3C(=O)COc4ccc(F)c(F)c4[C@H]4CC[C@H](CC4)OC[C@H]32)N1] | (1′s,3S,16′R,19′s)- 3′,4′-difluoro-8′,18′- dioxa-11′- azaspiro[morpholine- 3,15′- tetracyclo[17.2.2. 0²,⁷.0¹¹,¹⁶]tricosane]- 2′(7′),3′,5′-triene- 5,10′-dione | LCMS (Method B): [M + H]⁺ m/z 451.3, RT 2.88 minutes | ¹H NMR (400 MHz, CDCl₃) δ 6.96 − 6.81 (m, 1H), 6.76 (s, 1H), 6.69 (td, J = 9.0, 4.1 Hz, 1H), 5.45 − 5.35 (m, 2H), 4.38 − 4.25 (m, 2H), 4.18 − 3.99 (m, 3H), 3.95 − 3.78 (m, 2H), 3.76 (s, 1H), 3.59 − 3.50 (m, 1H), 3.37 (d, J = 11.7 Hz, 1H), 3.25 − 2.90 (m, 1H), 2.70 − 2.55 (m, 1H), 2.19 − 1.16 (m, 11H). |
| 43b | 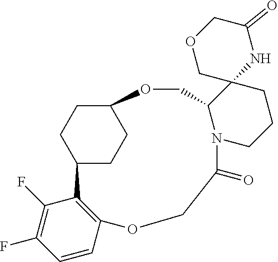 [SMILES: O=C1COC[C@]2(CCCN3C(=O)COc4ccc(F)c(F)c4[C@H]4CC[C@H](CC4)OC[C@@H]32)N1] | (1′s,3R,16′S,19′s)- 3′,4′-difluoro-8′,18′- dioxa-11′- azaspiro[morpholine- 3,15′- tetracyclo[17.2.2. 0²,⁷.0¹¹,¹⁶]tricosane]- 2′(7′),3′,5′-triene- 5,10′-dione | LCMS (Method B): [M + H]⁺ m/z 451.3, RT 2.87 minutes | ¹H NMR (400 MHz, CDCl₃) δ 6.87 (ddd, J = 10.9, 9.1, 5.1 Hz, 1H), 6.75 (s, 1H), 6.69 (td, J = 9.0, 4.0 Hz, 1H), 5.40 (dt, J = 10.8, 3.5 Hz, 2H), 4.39 − 4.24 (m, 2H), 4.14 (d, J = 11.7 Hz, 1H), 4.09 (d, J = 16.9 Hz, 1H), 3.95 − 3.80 (m, 2H), 3.76 (s, 1H), 3.57 − 3.43 (m, 2H), 3.37 (d, J = 11.7 Hz, 1H), 3.17 − 3.06 (m, 1H), 2.70 − 2.55 (m, 1H), 2.13 − 1.88 (m, 3H), 1.87 − 1.68 (m, 3H), 1.51 − 1.33 (m, 4H), 1.28 − 1.18 (m, 1H). |
| 44a | 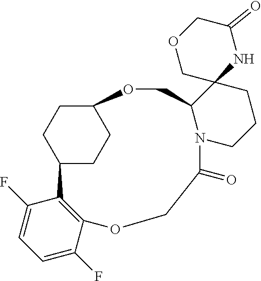 [SMILES: O=C1COC[C@@]2(CCCN3C(=O)COc4c(F)ccc(F)c4[C@H]4CC[C@H](CC4)OC[C@H]32)N1] | (1′s,3S,16′R,19′s)- 3′,6′-difluoro-8′,18′- dioxa-11′- azaspiro[morpholine- 3,15′- tetracyclo[17.2.2. 0²,⁷.0¹¹,¹⁶]tricosane]- 2′(7′),3′,5′-triene- 5,10′-dione | LCMS (Method B): [M + H]⁺ m/z 451.3, RT 2.87 minutes | ¹H NMR (500 MHz, CDCl₃) δ 6.91 − 6.81 (m, 1H), 6.74 − 6.61 (m, 2H), 5.47 − 5.32 (m, 2H), 4.38 − 4.25 (m, 2H), 4.20 − 4.05 (m, 2H), 3.96 − 3.79 (m, 2H), 3.78 − 3.72 (m, 1H), 3.58 − 3.44 (m, 2H), 3.37 (d, J = 11.7 Hz, 1H), 3.18 − 3.06 (m, 1H), 2.70 − 2.55 (m, 1H), 2.14 − 2.06 (m, 1H), 2.03 − 1.97 (m, 1H), 1.96 − 1.89 (m, 1H), 1.87 − 1.67 (m, 4H), 1.50 − 1.35 (m, 4H). |
| 44b | 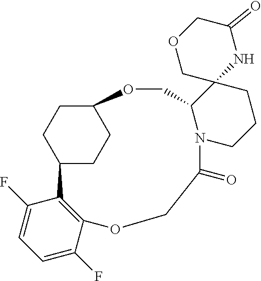 [SMILES: O=C1COC[C@]2(CCCN3C(=O)COc4c(F)ccc(F)c4[C@H]4CC[C@H](CC4)OC[C@@H]32)N1] | (1′s,3R,16′S,19′s)- 3′,6′-difluoro-8′,18′- dioxa-11′- azaspiro[morpholine- 3,15′- tetracyclo[17.2.2. 0²,⁷.0¹¹,¹⁶]tricosane]- 2′(7′),3′,5′-triene- 5,10′-dione | LCMS (Method B): [M + H]⁺ m/z 451.3, RT 2.88 minutes | ¹H NMR (500 MHz, CDCl₃) δ 6.91 − 6.83 (m, 1H), 6.69 (td, J = 9.0, 4.0 Hz, 1H), 6.51 (s, 1H), 5.45 − 5.31 (m, 2H), 4.37 − 4.27 (m, 2H), 4.16 − 4.05 (m, 2H), 3.91 (dd, J = 14.2, 4.5 Hz, 1H), 3.84 (dd, J = 10.5, 8.4 Hz, 1H), 3.79 − 3.71 (m, 1H), 3.58 − 3.45 (m, 2H), 3.38 (d, J = 11.7 Hz, 1H), 3.17 − 3.05 (m, 1H), 2.68 − 2.56 (m, 1H), 2.12 − 2.05 (m, 1H), 1.99 (dd, J = 12.6, 4.0 Hz, 1H), 1.95 − 1.89 (m, 1H), 1.87 − 1.69 (m, 4H), 1.51 − 1.35 (m, 4H). |
| 45 | 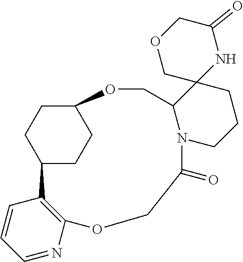 [SMILES: O=C1COCC2(CCCN3C(=O)COc4ncccc4[C@H]4CC[C@H](CC4)OCC32)N1] | Rel- (1′s,3S,16′R,19′s)- 8′,18′-dioxa-6′,11′- diazaspiro [morpholine-3,15′- tetracyclo[17.2.2. 0²,⁷.0¹¹,¹⁶]tricosane]- 2′(7′),3′,5′-triene- 5,10′-dione | LCMS (Method B): [M + H]⁺ m/z 416.3, RT 2.21 minutes | ¹H NMR (500 MHz, CDCl₃) δ 7.98 (dd, J = 5.1, 1.9 Hz, 1H), 7.37 (dd, J = 7.2, 1.9 Hz, 1H), 6.83 (dd, J = 7.1, 5.0 Hz, 1H), 6.49 (s, 1H), 5.37 (dd, J = 10.3, 4.2 Hz, 1H), 5.18 (d, J = 11.3 Hz, 1H), 4.61 (d, J = 11.3 Hz,1H), 4.32 − 4.20 (m, 1H), 4.18 − 4.08 (m, 2H), 3.92 − 3.85 (m, 1H), 3.76 − 3.64 (m, 2H), 3.56 (dd, J = 8.6, 4.2 Hz, 1H), 3.54 − 3.48 (m, 1H), 3.40 (d, J = 11.7 Hz, 1H), 2.64 − 2.50 (m, 2H), 2.36 − 2.25 (m, 1H), 2.14 − 2.03 (m, 1H), 1.91 − 1.75 (m, 4H), 1.48 − 1.26 (m, 5H). |
| 45a | 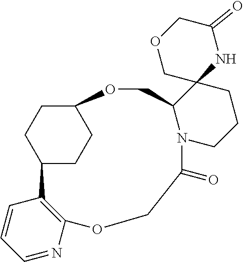 [SMILES: O=C1COC[C@@]2(CCCN3C(=O)COc4ncccc4[C@H]4CC[C@H](CC4)OC[C@H]32)N1] | (1′s,3S,16′R,19′s)- 8′,18′-dioxa-6′,11′- diazaspiro [morpholine-3,15′- tetracyclo[17.2.2. 0²,⁷.0¹¹,¹⁶]tricosane]- 2′(7′),3′,5′-triene- 5,10′-dione | LCMS (Method C): [M + H]⁺ m/z 416.4, RT 0.78 minutes | ¹H NMR (500 MHz, CDCl₃) δ 8.01 (dd, J = 5.1, 1.6 Hz, 1H), 7.42 (dd, J = 7.1, 1.4 Hz, 1H), 6.87 (dd, J = 7.1, 5.1 Hz, 1H), 6.77 (s, 1H), 5.39 (dd, J = 10.3, 3.8 Hz, 1H), 5.28 (br d, J = 11.3 Hz, 1H), 4.64 (d, J = 11.3 Hz, 1H), 4.29 (d, J = 17.0 Hz, 1H), 4.17 (d, J = 11.8 Hz, 1H), 4.10 (d, J = 16.9 Hz, 1H), 3.90 (dd, J = 10.0, 8.9 Hz, 1H), 3.67 − 3.77 (m, 2H), 3.56 (dd, J = 8.6, 4.1 Hz, 1H), 3.50 (td, J = 13.4, 2.7 Hz, 1H), 3.40 (d, J = 11.7 Hz, 1H), 2.51 − 2.68 (m, 2H), 2.31 (qd, J = 12.8, 4.1 Hz, 1H), 2.04 − 2.15 (m, 1H), 1.24 − 1.98 (m, 9H). |
| 45b | 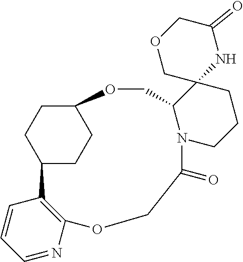 [SMILES: O=C1COC[C@]2(CCCN3C(=O)COc4ncccc4[C@H]4CC[C@H](CC4)OC[C@@H]32)N1] | (1′s,3R,16′S,19′s)- 8′,18′-dioxa-6′,11′- diazaspiro [morpholine-3,15′- tetracyclo[17.2.2. 0²,⁷.0¹¹,¹⁶]tricosane]- 2′(7′),3′,5′-triene- 5,10′-dione | LCMS (Method B): [M + H]⁺ m/z 434.3, RT 2.38 minutes | ¹H NMR (500 MHz, CDCl₃) δ 8.01 (dd, J = 5.1, 1.6 Hz, 1H), 7.42 (dd, J = 7.1, 1.4 Hz, 1H), 6.87 (dd, J = 7.1, 5.1 Hz, 1H), 6.77 (s, 1H), 5.39 (dd, J = 10.3, 3.8 Hz, 1H), 5.28 (br d, J = 11.3 Hz, 1H), 4.64 (d, J = 11.3 Hz, 1H), 4.29 (d, J = 17.0 Hz, 1H), 4.17 (d, J = 11.8 Hz, 1H), 4.10 (d, J = 16.9 Hz, 1H), 3.90 (dd, J = 10.0, 8.9 Hz, 1H), 3.67 − 3.77 (m, 2H), 3.56 (dd, J = 8.6, 4.1 Hz, 1H), 3.50 (td, J = 13.4, 2.7 Hz, 1H), 3.40 (d, J = 11.7 Hz, 1H), 2.51 − 2.68 (m, 2H), 2.31 (qd, J = 12.8, 4.1 Hz, 1H), 2.04 − 2.15 (m, 1H), 1.24 − 1.98 (m, 9H). |
| 46a | 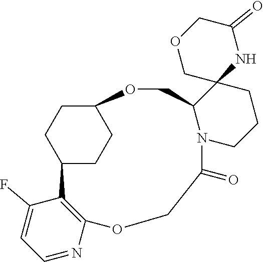 [SMILES: O=C1COC[C@@]2(CCCN3C(=O)COc4nccc(F)c4[C@H]4CC[C@H](CC4)OC[C@H]32)N1] | (1′s,3S,16′R,19′s)- 3′-fluoro-8′,18′- dioxa-6′,11′- diazaspiro [morpholine- 3,15′- tetracyclo[17.2.2. 0²,⁷.0¹¹,¹⁶]tricosane]- 2′(7′),3′,5′-triene- 5,10′-dione | LCMS (Method B): [M + H]⁺ m/z 434.3, RT 2.38 minutes | ¹H NMR (500 MHz, CDCl₃) δ 7.95 − 7.85 (m, 1H), 6.70 − 6.61 (m, 1H), 6.34 (s, 1H), 5.47 − 5.33 (m, 1H), 5.17 (d, J = 11.3 Hz, 1H), 4.77 − 4.56 (m, 1H), 4.33 − 4.22 (m, 1H), 4.20 − 4.04 (m, 2H), 3.93 − 3.80 (m, 1H), 3.76 − 3.64 (m, 2H), 3.57 (dd, J = 8.6, 4.2 Hz, 1H), 3.53 − 3.44 (m, 1H), 3.42 − 3.27 (m, 1H), 3.19 − 3.07 (m, 1H), 2.57 − 2.45 (m, 1H), 2.32 − 2.19 (m, 1H), 2.08 (t, J = 12.4 Hz, 1H), 1.95 − 1.82 (m, 2H), 1.81 − 1.63 (m, 3H), 1.50 − 1.34 (m, 4H). |
| 46b | 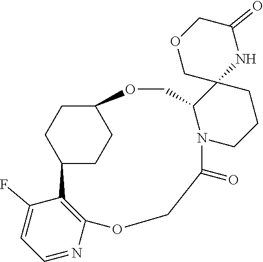 [SMILES: O=C1COC[C@]2(CCCN3C(=O)COc4nccc(F)c4[C@H]4CC[C@H](CC4)OC[C@@H]32)N1] | (1′s,3R,16′S,19′s)- 3′-fluoro-8′,18′- dioxa-6′,11′- diazaspiro [morpholine-3,15′- tetracyclo[17.2.2. 0²,⁷.0¹¹,¹⁶]tricosane]- 2′(7′),3′,5′-triene- 5,10′-dione | LCMS (Method B): [M + H]⁺ m/z 434.3, RT 2.38 minutes | ¹H NMR (500 MHz, CDCl₃) δ 7.96 − 7.86 (m, 1H), 6.71 − 6.59 (m, 1H), 6.43 (s, 1H), 5.49 − 5.32 (m, 1H), 5.17 (d, J = 11.3 Hz, 1H), 4.74 − 4.56 (m, 1H), 4.34 − 4.23 (m, 1H), 4.20 − 4.02 (m, 2H), 3.94 − 3.81 (m, 1H), 3.75 − 3.63 (m, 2H), 3.57 (dd, J = 8.6, 4.2 Hz, 1H), 3.54 − 3.45 (m, 1H), 3.42 − 3.26 (m, 1H), 3.20 − 3.07 (m, 1H), 2.58 − 2.43 (m, 1H), 2.32 − 2.20 (m, 1H), 2.17 − 2.04 (m, 1H), 1.93 − 1.83 (m, 2H), 1.81 − 1.62 (m, 3H), 1.47 − 1.36 (m, 4H). |
| 47 | 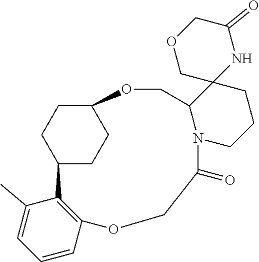 [SMILES: Cc1cccc2c1[C@H]1CC[C@H](CC1)OCC1N(CCCC13COCC(=O)N3)C(=O)CO2] | Rel- (1′s,3S,16′R,19′s)- 3′-methyl-8′,18′- dioxa-11′- azaspiro[morpholine- 3,15′- tetracyclo[17.2.2. 0²,⁷.0¹¹,¹⁶]tricosane]- 2′(7′),3′,5′-triene- 5,10′-dione | LCMS (Method C): [M + H]⁺ m/z 429.3, RT 0.96 minutes | ¹H NMR (400 MHz, CDCl₃) δ 7.04 (t, J = 7.9 Hz, 1H), 6.80 (d, J = 7.6 Hz, 1H), 6.55 (d, J = 8.7 Hz, 1H), 6.30 (s, 1H), 5.39 (dd, J = 10.4, 3.9 Hz, 1H), 5.07 (d, J = 10.4 Hz, 1H), 4.27 − 4.35 (m, 2H), 4.07 − 4.19 (m, 2H), 3.88 (dd, J = 10.3, 8.7 Hz, 1H), 3.66 − 3.77 (m, 2H), 3.56 (dd, J = 8.4, 4.1 Hz, 1H), 3.46 − 3.53 (m, 1H), 3.41 (d, J = 11.7 Hz, 1H), 2.88 − 3.00 (m, 1H), 2.57 − 2.74 (m, 1H), 2.32 (s, 3H), 2.19 − 2.37 (m, 1H), 2.05 − 2.17 (m, 1H), 1.81 − 1.98 (m, 2H), 1.69 − 1.80 (m, 2H), 1.51 − 1.66 (m, 1H), 1.20 − 1.48 (m, 4H). |
| 47a | 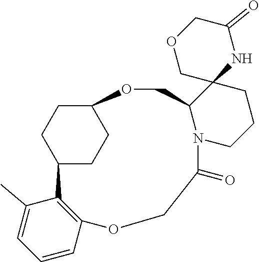 [SMILES: Cc1cccc2c1[C@H]1CC[C@H](CC1)OC[C@@H]1N(CCC[C@@]13COCC(=O)N3)C(=O)CO2] | (1′s,3S,16′R,19′s)- 3′-methyl-8′,18′- dioxa-11′- azaspiro[morpholine- 3,15′- tetracyclo[17.2.2. 0²,⁷.0¹¹,¹⁶]tricosane]- 2′(7′),3′,5′-triene- 5,10′-dione | LCMS (Method C): [M + H]⁺ m/z 429.2, RT 0.96 minutes | ¹H NMR (500 MHz, CDCl₃) δ 7.04 (t, J = 7.8 Hz, 1H), 6.80 (d, J = 7.3 Hz, 1H), 6.61 (d, J = 8.0 Hz, 1H), 6.29 (br s, 1H), 5.39 (dd, J = 10.4, 3.9 Hz, 1H), 5.07 (d, J = 10.3 Hz, 1H), 4.26 − 4.33 (m, 2H), 4.08 − 4.18 (m, 2H), 3.88 (dd, J = 10.3, 8.8 Hz, 1H), 3.65 − 3.79 (m, 2H), 3.46 − 3.60 (m, 2H), 3.41 (d, J = 11.7 Hz, 1H), 2.89 − 2.99 (m, 1H), 2.56 − 2.74 (m, 1H), 2.32 (s, 3H), 2.21 − 2.37 (m, 1H), 2.09 (br d, J = 12.6 Hz, 1H), 1.89 − 1.98 (m, 1H), 1.69 − 1.88 (m, 3H), 1.54 − 1.65 (m, 1H), 1.21 − 1.51 (m, 4H). |
| 47b | 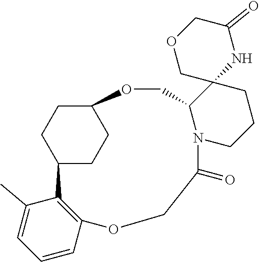 [SMILES: Cc1cccc2c1[C@H]1CC[C@H](CC1)OC[C@H]1N(CCC[C@]13COCC(=O)N3)C(=O)CO2] | (1′s,3R,16′S,19′s)- 3′-methy1-8′,18′- dioxa-11′- azaspiro[morpholine- 3,15′- tetracyclo[17.2.2. 0²,⁷.0¹¹,¹⁶]tricosane]- 2′(7′),3′,5′-triene- 5,10′-dione | LCMS (Method C): [M + H]⁺ m/z 429.3, RT 0.96 minutes | ¹H NMR (500 MHz, CDCl₃) δ 7.04 (t, J = 7.8 Hz, 1H), 6.80 (d, J = 7.3 Hz, 1H), 6.61 (d, J = 8.0 Hz, 1H), 6.29 (br s, 1H), 5.39 (dd, J = 10.4, 3.9 Hz, 1H), 5.07 (d, J = 10.3 Hz, 1H), 4.26 − 4.33 (m, 2H), 4.08 − 4.18 (m, 2H), 3.88 (dd, J = 10.3, 8.8 Hz, 1H), 3.65 − 3.79 (m, 2H), 3.46 − 3.60 (m, 2H), 3.41 (d, J = 11.7 Hz, 1H), 2.89 − 2.99 (m, 1H), 2.56 − 2.74 (m, 1H), 2.32 (s, 3H), 2.21 − 2.37 (m, 1H), 2.09 (br d, J = 12.6 Hz, 1H), 1.89 − 1.98 (m, 1H), 1.69 − 1.88 (m, 3H), 1.54 − 1.65 (m, 1H), 1.21 − 1.51 (m, 4H). |

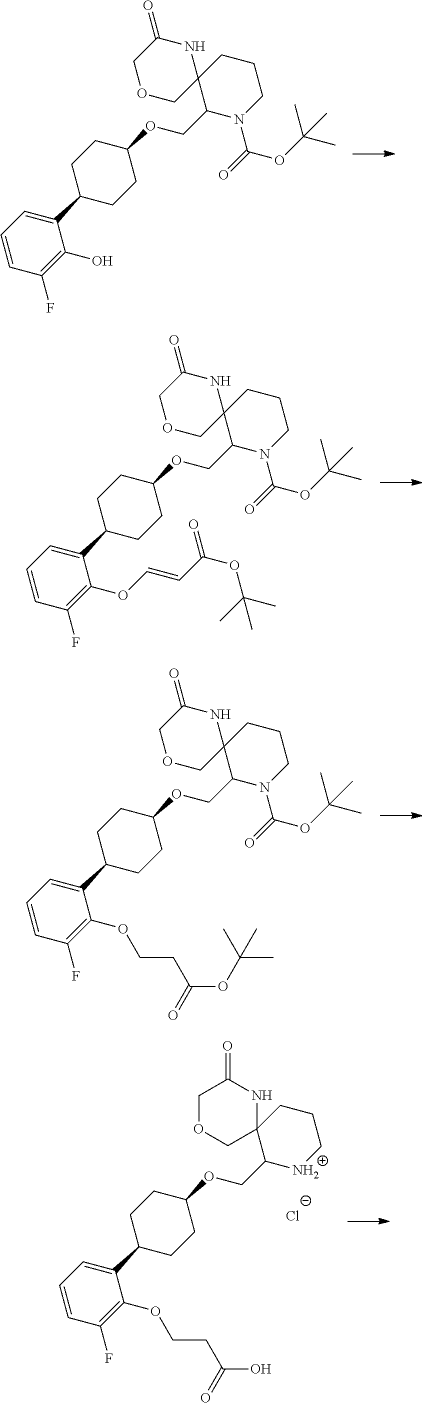 [SMILES: CC(C)(C)OC(=O)N1CCCC2(COCC(=O)N2)C1CO[C@H]1CC[C@@H](c2cccc(F)c2O)CC1 ; CC(C)(C)OC(=O)/C=C/Oc1c(F)cccc1[C@H]1CC[C@@H](OCC2N(C(=O)OC(C)(C)C)CCCC23COCC(=O)N3)CC1 ; CC(C)(C)OC(=O)CCOc1c(F)cccc1[C@H]1CC[C@@H](OCC2N(C(=O)OC(C)(C)C)CCCC23COCC(=O)N3)CC1 ; O=C(O)CCOc1c(F)cccc1[C@H]1CC[C@@H](OCC2[NH2+]CCCC23COCC(=O)N3)CC1 ; [Cl-]]

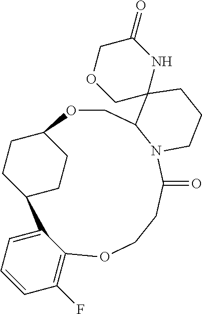 [SMILES: O=C1COCC2(CCCN3C(=O)CCOc4c(F)cccc4[C@H]4CC[C@H](CC4)OCC32)N1]

### Intermediate 99

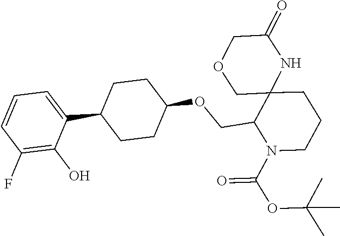 [SMILES: CC(C)(C)OC(=O)N1CCCC2(COCC(=O)N2)C1CO[C@H]1CC[C@@H](c2cccc(F)c2O)CC1]

### tert-butyl-rel-(6S, 7R)-2-oxo-7-({[(1s,4s)-4-(3-fluoro-2-hydroxyphenyl)cyclohexyl]-oxy}methyl)-4-oxa-1,8-diazaspiro[5.5]undecane-8-carboxylate

Intermediate 99 was prepared using commercially available starting material 1-tert-butyl 4-ethyl 3-oxopiperidine-1,4-dicarboxylate following a similar procedure as described for examples 17a and 17b to afford the title compound (1.80 g) as a yellow oil. M+H]⁺ m/z 493.4.

### Intermediate 100

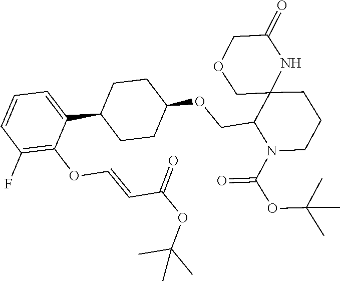 [SMILES: CC(C)(C)OC(=O)/C=C/Oc1c(F)cccc1[C@H]1CC[C@@H](OCC2N(C(=O)OC(C)(C)C)CCCC23COCC(=O)N3)CC1]

### tert-butyl-rel-(6S,7R)-2-oxo-7-({[(1s,4s)-4-(2-{[(1E)-3-(tert-butoxy)-3-oxoprop-1-en-1-yl]oxy}-3-fluorophenyl)cyclohexyl]oxy}methyl)-4-oxa-1,8-diazaspiro[5.5]undecane-8-carboxylate

To a solution of tert-butyl prop-2-ynoate (334 μL, 2.43 mmol) and 1,4-diazabicyclo[2.2.2]octane (89 mg, 0.793 mmol) in THF (1.6 mL) at 0° C. under nitrogen was added Intermediate 99 (1.00 g) in THF (8.4 mL) and the solution was stirred at room temperature for 5 hours. The reaction mixture was quenched with water (20 mL) and extracted with EtOAc (2×25 mL). The combined organic phases were washed with brine, dried over MgSO₄ and concentrated in vacuo to afford the crude. The crude was purified by flash column chromatography (0-100% EtOAc in heptane) to afford the title compound (1.00 g) as a yellow oil. [M+H]⁺ m/z 619.5

### Intermediate 101

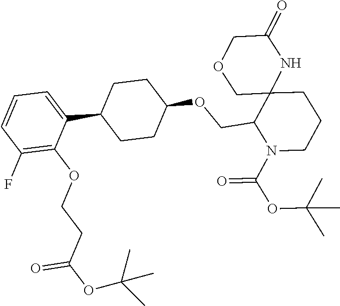 [SMILES: CC(C)(C)OC(=O)CCOc1c(F)cccc1[C@H]1CC[C@@H](OCC2N(C(=O)OC(C)(C)C)CCCC23COCC(=O)N3)CC1]

### tert-butyl-rel-(6S,7R)-2-oxo-7-({[(1s,4s)-4-{2-[3-(tert-butoxy)-3-oxopropoxy]-3-fluorophenyl}cyclohexyl]oxy}methyl)-4-oxa-1,8-diazaspiro[5.5]undecane-8-carboxylate

Intermediate 100 (1.00 g) was dissolved in ethanol (27 mL) and the atmosphere was evacuated and backfilled with nitrogen 3 times. Palladium on carbon (10%, 516 mg, 0.485 mmol) was added and the atmosphere was evacuated and backfilled with hydrogen 3 times. The reaction was stirred for 16 hours and then filtered through Celite, washing with EtOAc and the filtrate was concentrated in vacuo to afford the crude. The crude material was purified by flash column chromatography (0-100% EtOAc in heptane) to afford the title compound (800 g) as a yellow oil. [M+H]⁺ m/z 621.5.

### Intermediate 102

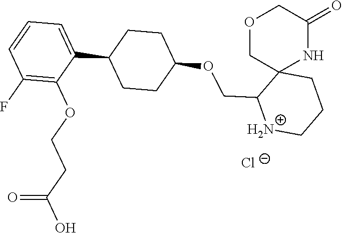 [SMILES: O=C(O)CCOc1c(F)cccc1[C@H]1CC[C@@H](OCC2[NH2+]CCCC23COCC(=O)N3)CC1 ; [Cl-]]

### Rel-(6S, 7R)-2-oxo-7-({[(1s,4s)-4-[2-(2-carboxyethoxy)-3-fluorophenyl]cyclohexyl]oxy}methyl)-4-oxa-1,8-diazaspiro[5.5]undecan-8-ium chloride

4 M Hydrogen chloride in dioxane (3.2 mL, 12.8 mmol) was added to Intermediate 101 (800 mg) at room temperature and the reaction was stirred for 2 hours. The reaction mixture was concentrated in vacuo to afford the title compound (650 mg) as an orange solid. [M+H]⁺ m/z 465.4

### Example 48

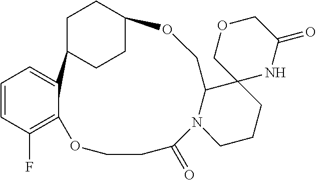 [SMILES: O=C1COCC2(CCCN3C(=O)CCOc4c(F)cccc4[C@H]4CC[C@H](CC4)OCC32)N1]

### Rel-(1′s,3S,17′R,20's)-6′-fluoro-8′,19′-dioxa-12′-azaspiro[morpholine-3,16′-tetracyclo[18.2.2.0^{2,7}0.0^{12,17}]tetracosane]-2′,4′,6′-triene-5,11′-dione

To a stirred solution of 2,4,6-tripropyl-1,3,5,2,4,6-trioxatriphosphinane 2,4,6-trioxide (50%, 1.7 mL, 2.96 mmol) and N-ethyl-N-(propan-2-yl)propan-2-amine (460 mg, 3.56 mmol) in acetonitrile (110.15 mL) was added Intermediate 102 (550 mg) in anhydrous DMF (4.2367 mL) over 2h using a syringe pump. The resulting solution was stirred for one hour. The reaction mixture was concentrated in vacuo to afford the crude material. The crude material was diluted in water and extracted with DCM (3×20 mL) The combined organic extracts were dried (MgSO₄), filtered and concentrated to give a solid residue. The crude material was purified by basic reverse phase column chromatography (20-50% acetonitrile in water (0.1% ammonia)) to afford the title compound (75 mg) as a white solid. [M+H]⁺ m/z 447.4

### Examples 48a and 48b

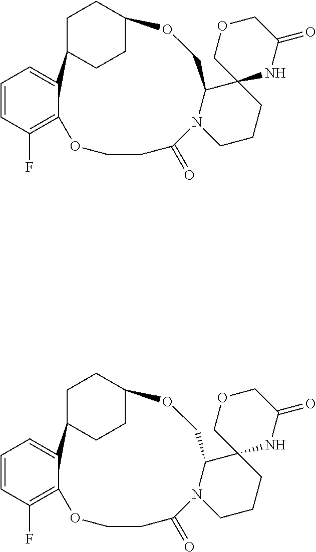 [SMILES: O=C1COC[C@@]2(CCCN3C(=O)CCOc4c(F)cccc4[C@H]4CC[C@H](CC4)OC[C@H]32)N1 ; O=C1COC[C@]2(CCCN3C(=O)CCOc4c(F)cccc4[C@H]4CC[C@H](CC4)OC[C@@H]32)N1]

### Example 48a: (1′s,3S, 17′R,20′s)-6′-fluoro-8′,19′-dioxa-12′-azaspiro[morpholine-3,16′-tetracyclo[18.2.2.0^{2,7}0.0^{12,17}]tetracosane]-2′(7′),3′,5′-triene-5,11′-dione

### Example 48b: (1′s,3R,17'S,20's)-6′-fluoro-8′,19′-dioxa-12′-azaspiro[morpholine-3,16′-tetracyclo[18.2.2.0^{2,7}0.0^{12,17}]tetracosane]-2′(7′),3′,5′-triene-5,11′-dione

Example 48 (75 mg) was subjected to chiral preparative purification using Waters 600 eluting with 40/60% v/v n-Hexane/ethanol+0.1% isopropylamine), Chiralpak IC (25×2.0 cm), 5 μm, flow rate 17 mL/minute to afford the title compounds (Peak 1, 29 mg, 100% ee; and Peak 2, 31 mg, 96.7% ee).

### Example 48a: Peak 1 (Stereochemistry Tentatively Assigned)

LCMS (Method C): [M+H]⁺ m/z 447.4, RT 0.93 minutes.

Chiral analysis (Chiralpak IC, 25×0.46 cm, 5 μm, 40:60 n-Hexane: ethanol+0.1% isopropylamine): RT 7.4 minutes

¹H NMR (600 MHZ, CDCl₃) δ 6.98-6.71 (m, 3H), 6.26-6.08 (m, 1H), 5.47-4.65 (m, 1H), 5.19-4.48 (m, 1H), 4.37-4.24 (m, 2H), 4.84-4.04 (m, 1H), 4.19-4.02 (m, 2H), 3.84 (dt, J=12.6, 9.4 Hz, 1H), 3.77-3.66 (m, 1H), 3.72-3.62 (m, 1H), 3.60-3.31 (m, 1H), 3.39-3.25 (m, 1H), 3.43-2.66 (m, 1H), 2.56-2.32 (m, 2H), 2.55-1.22 (m, 12H).

### Example 48b: Peak 2 (Stereochemistry Tentatively Assigned)

LCMS (Method C): [M+H]⁺ m/z 447.4, RT 0.93 minutes.

Chiral analysis (Chiralpak IC, 25×0.46 cm, 5 μm, 40:60 n-Hexane: ethanol+0.1% isopropylamine): RT 8.6 minutes

¹H NMR (600 MHZ, CDCl₃) δ 6.98-6.71 (m, 3H), 6.26-6.08 (m, 1H), 5.47-4.65 (m, 1H), 5.19-4.48 (m, 1H), 4.37-4.24 (m, 2H), 4.84-4.04 (m, 1H), 4.19-4.02 (m, 2H), 3.84 (dt, J=12.6, 9.4 Hz, 1H), 3.77-3.66 (m, 1H), 3.72-3.62 (m, 1H), 3.60-3.31 (m, 1H), 3.39-3.25 (m, 1H), 3.43-2.66 (m, 1H), 2.56-2.32 (m, 2H), 2.55-1.22 (m, 12H).

Below examples were prepared following analogous procedures as described for Examples 48a and 48b using the appropriate reagents

| | | | Obs. | |
|---|---|---|---|---|
| Ex. | Structure | Name | Mass | ¹H NMR |
| 49 | 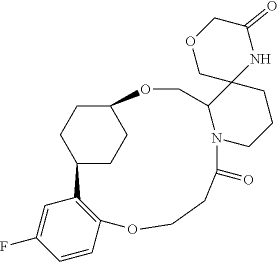 [SMILES: O=C1COCC2(CCCN3C(=O)CCOc4ccc(F)cc4[C@H]4CC[C@H](CC4)OCC32)N1] | Rel- (1′s,3S,17′R,20′s)- 4′-fluoro-8′,19′- dioxa-12′- azaspiro[morpholine- 3,16′- tetracyclo[18.2.2. 0²,⁷.0¹²,¹⁷]tetracosane]- 2′(7′),3′,5′-triene- 5,11′-dione | LCMS (Method B): [M + H]⁺ m/z 447.4, RT 3.03 minutes | ¹H NMR (400 MHz, CDCl₃) δ 6.84 − 6.66 (m, 3H), 6.31 (s, 1H), 4.83 − 4.64 (m, 3H), 4.33 (d, J = 17.0 Hz, 1H), 4.19 − 4.08 (m, 3H), 4.08 − 3.94 (m, 1H), 3.85 (t, J = 9.7 Hz, 1H), 3.74 (dd, J = 9.1, 2.8 Hz, 1H), 3.64 − 3.60 (m, 1H), 3.54 − 3.42 (m, 1H), 3.27 (d, J = 12.0 Hz, 1H), 2.76 − 2.65 (m, 1H), 2.44 − 2.31 (m, 2H), 2.26 (d, J = 15.1 Hz, 1H), 2.01-1.19 (m, 10H). |
| 49a | 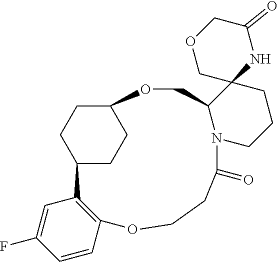 [SMILES: O=C1COC[C@@]2(CCCN3C(=O)CCOc4ccc(F)cc4[C@H]4CC[C@H](CC4)OC[C@H]32)N1] | (1′s,3S,17′R,20′s)- 4′-fluoro-8′,19′- dioxa-12′- azaspiro[morpholine- 3,16′- tetracyclo[18.2.2. 0²,⁷.0¹²,¹⁷]tetracosane]- 2′(7′),3′,5′-triene- 5,11′-dione | LCMS (Method C): [M + H]⁺ m/z 447.3, RT 0.95 minutes | ¹H NMR (500 MHz, CDCl₃) δ 6.78 − 6.83 (m, 1H), 6.74 − 6.78 (m, 1H), 6.69 − 6.73 (m, 1H), 6.19 (br s, 1H), 4.80 (br dd, J = 14.1, 4.2 Hz, 1H), 4.65 − 4.76 (m, 2H), 4.34 (d, J = 17.0 Hz, 1H), 4.14 − 4.19 (m, 1H), 4.09 − 4.15 (m, 1H), 3.98 (dt, J = 7.6, 2.8 Hz, 1H), 3.86 (t, J = 9.7 Hz, 1H), 3.75 (dd, J = 9.1, 2.5 Hz, 1H), 3.60 − 3.66 (m, 1H), 3.44 − 3.53 (m, 1H), 3.28 (d, J = 11.9 Hz, 1H), 2.71 (td, J = 13.6, 3.2 Hz, 1H), 2.54 − 2.66 (m, 1H), 2.33 − 2.52 (m, 2H), 2.27 (br d, J = 15.6 Hz, 1H), 1.19 − 2.01 (m, 10H). |
| 49b | 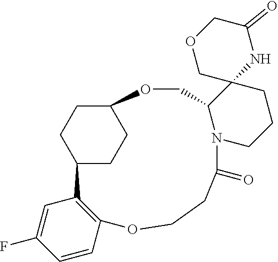 [SMILES: O=C1COC[C@]2(CCCN3C(=O)CCOc4ccc(F)cc4[C@H]4CC[C@H](CC4)OC[C@@H]32)N1] | (1′s,3R,17′S,20′s)- 4′-fluoro-8′,19′- dioxa-12′- azaspiro[morpholine- 3,16′- tetracyclo[18.2.2. 0²,⁷.0¹²,¹⁷]tetracosane]- 2′(7′),3′,5′-triene- 5,11′-dione | LCMS (Method C): [M + H]⁺ m/z 447.4, RT 0.95 minutes | ¹H NMR (500 MHz, CDCl₃) δ 6.78 − 6.83 (m, 1H), 6.74 − 6.78 (m, 1H), 6.69 − 6.73 (m, 1H), 6.19 (br s, 1H), 4.80 (br dd, J = 14.1, 4.2 Hz, 1H), 4.65 − 4.76 (m, 2H), 4.34 (d, J = 17.0 Hz, 1H), 4.14 − 4.19 (m, 1H), 4.09 − 4.15 (m, 1H), 3.98 (dt, J = 7.6, 2.8 Hz, 1H), 3.86 (t, J = 9.7 Hz, 1H), 3.75 (dd, J = 9.1, 2.5 Hz, 1H), 3.60 − 3.66 (m, 1H), 3.44 − 3.53 (m, 1H), 3.28 (d, J = 11.9 Hz, 1H), 2.71 (td, J = 13.6, 3.2 Hz, 1H), 2.54 − 2.66 (m, 1H), 2.33 − 2.52 (m, 2H), 2.27 (br d, J = 15.6 Hz, 1H), 1.19 − 2.01 (m, 10H). |
| 50a | 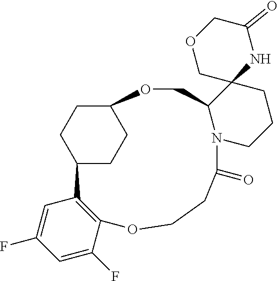 [SMILES: O=C1COC[C@@]2(CCCN3C(=O)CCOc4c(F)cc(F)cc4[C@H]4CC[C@H](CC4)OC[C@H]32)N1] | (1′s,3S,17′R,20′s)- 4′,6′-difluoro-8′,19′- dioxa-12′- azaspiro[morpholine- 3,16′- tetracyclo[18.2.2. 0²,⁷.0¹²,¹⁷]tetracosane]- 2′(7′),3′,5′-triene- 5,11′-dione | LCMS (Method C): [M + H]⁺ m/z 465.4, RT 0.94 minutes | ¹H NMR (400 MHz, CDCl₃) δ 6.75 − 6.62 (m, 1H), 6.64 − 6.51 (m, 1H), 6.07 − 5.92 (m, 1H), 5.50 − 4.62 (m, 1H), 5.15 − 4.40 (m, 1H), 4.38 − 4.19 (m, 2H), 4.19 − 4.02 (m, 2H), 4.86 − 4.01 (m, 1H), 3.89 − 3.78 (m, 1H), 3.76 − 3.62 (m, 2H), 3.61 − 3.35 (m, 1H), 3.43 − 3.21 (m, 1H), 3.43 − 2.63 (m, 1H), 2.58 − 2.22 (m, 2H), 2.46 − 1.23 (m, 12H). |
| 50b | 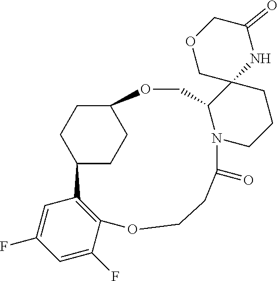 [SMILES: O=C1COC[C@]2(CCCN3C(=O)CCOc4c(F)cc(F)cc4[C@H]4CC[C@H](CC4)OC[C@@H]32)N1] | (1′s,3R,17′S,20′s)- 4′,6′-difluoro-8′,19′- dioxa-12′- azaspiro[morpholine- 3,16′- tetracyclo[18.2.2. 0²,⁷.0¹²,¹⁷]tetracosane]- 2′(7′),3′,5′-triene- 5,11′-dione | LCMS (Method C): [M + H]⁺ m/z 465.4, RT 0.94 minutes | ¹H NMR (400 MHz, CDCl₃) δ 6.75 − 6.62 (m, 1H), 6.64 − 6.51 (m, 1H), 6.07 − 5.92 (m, 1H), 5.50 − 4.62 (m, 1H), 5.15 − 4.40 (m, 1H), 4.38 − 4.19 (m, 2H), 4.19 − 4.02 (m, 2H), 4.86 − 4.01 (m, 1H), 3.89 − 3.78 (m, 1H), 3.76 − 3.62 (m, 2H), 3.61 − 3.35 (m, 1H), 3.43 − 3.21 (m, 1H), 3.43 − 2.63 (m, 1H), 2.58 − 2.22 (m, 2H), 2.46 − 1.23 (m, 12H). |
| 51 | 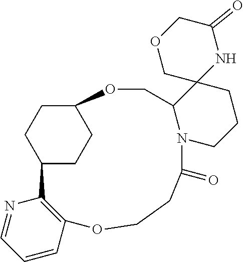 [SMILES: O=C1COCC2(CCCN3C(=O)CCOc4cccnc4[C@H]4CC[C@H](CC4)OCC32)N1] | Rel- (1′s,3S,17′R,20′s)- 8′,19′-dioxa-3′,12′- diazaspiro [morpholine- 3,16′- tetracyclo[18.2.2. 0²,⁷.0¹²,¹⁷]tetracosane]- 2′(7′),3′,5′-triene- 5,11′-dione | LCMS (Method C): [M + H]⁺ m/z 430.3, RT 0.62 minutes | ¹H NMR (400 MHz, CDCl₃) δ 8.00 (dd, J = 3.7, 2.4 Hz, 1H), 7.04 − 7.12 (m, 2H), 6.60 (s, 1H), 4.65 − 4.86 (m, 3H), 4.34 (d, J = 17.0 Hz, 1H), 4.08 − 4.21 (m, 2H), 3.99 − 4.07 (m, 1H), 3.82 − 3.93 (m, 1H), 3.77 (dd, J = 9.3, 2.6 Hz, 1H), 3.66 (br s, 1H), 3.46 − 3.59 (m, 1H), 3.28 (d, J = 12.1 Hz, 1H), 2.79 − 2.93 (m, 1H), 2.68 − 2.79 (m, 1H), 2.31 (dd, J = 15.6, 1.3 Hz, 1H), 1.29 − 2.67 (m, 12H). |

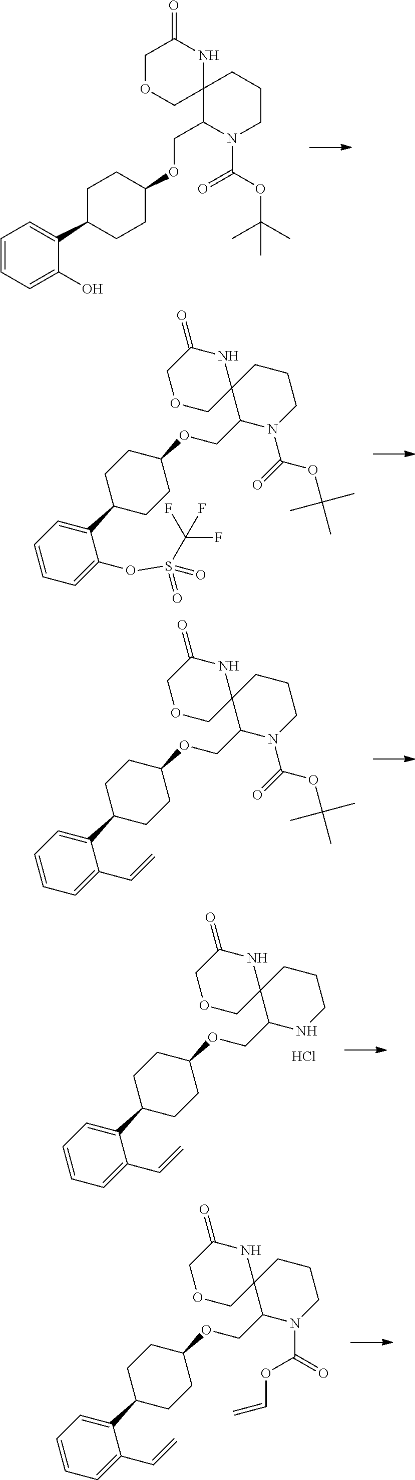

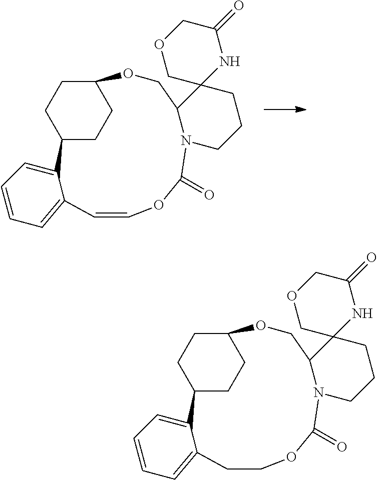 [SMILES: O=C1COCC2(CCCN3C(=O)O/C=C\c4ccccc4[C@H]4CC[C@H](CC4)OCC32)N1 ; O=C1COCC2(CCCN3C(=O)OCCc4ccccc4[C@H]4CC[C@H](CC4)OCC32)N1]

### Intermediate 103

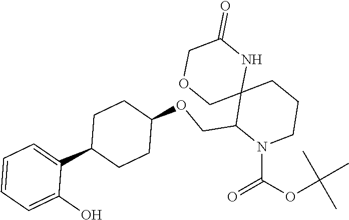 [SMILES: CC(C)(C)OC(=O)N1CCCC2(COCC(=O)N2)C1CO[C@H]1CC[C@@H](c2ccccc2O)CC1]

### tert-butyl-rel-(6S,7R)-2-oxo-7-({[(1s,4s)-4-(2-hydroxyphenyl)cyclohexyl]oxy}methyl)-4-oxa-1,8-diazaspiro[5.5]undecane-8-carboxylate

Intermediate 103 was prepared using commercially available starting material 1-tert-butyl 4-ethyl 3-oxopiperidine-1,4-dicarboxylate following a similar procedure as described in example 3 to afford the title compound (1.6 g) as a white solid. [M+H]⁺ m/z 475.3

### Intermediate 104

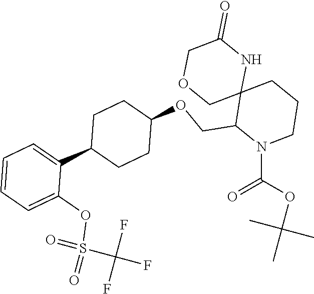 [SMILES: CC(C)(C)OC(=O)N1CCCC2(COCC(=O)N2)C1CO[C@H]1CC[C@@H](c2ccccc2OS(=O)(=O)C(F)(F)F)CC1]

### tert-butyl-rel-(6S,7R)-2-oxo-7-({[(1s,4s)-4-[2-(trifluoromethanesulfonyloxy)-phenyl]cyclohexyl]oxy}methyl)-4-oxa-1,8-diazaspiro[5.5]undecane-8-carboxylate

Trifluoromethylsulfonyl trifluoromethanesulfonate (0.38 mL, 2.02 mmol) was added dropwise in DCM (2 ml) to a stirred solution of Intermediate 103 (800 mg) and pyridine (0.41 mL, 5.06 mmol) in DCM (20 mL) at 0° C. The mixture was stirred at 0° C. for 4 hrs. The reaction mixture was diluted with DCM (20 mL) and water (20 mL), organic layer was separated and the aqueous layer was extracted with DCM (2×20 ml). The combined organic extracts were dried (Na₂SO₄) and filtered. The filtrate was concentrated in vacuo to afford the crude material. The crude material was purified by column chromatography (0-100% EtOAc in heptane) to afford the title compound (742 mg) as a yellow solid. [M+H]⁺ m/z 607.1

### Intermediate 105

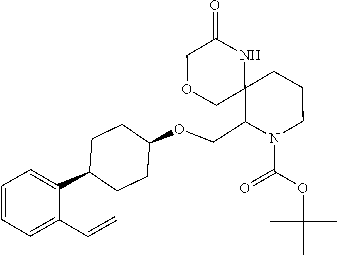 [SMILES: C=Cc1ccccc1[C@H]1CC[C@@H](OCC2N(C(=O)OC(C)(C)C)CCCC23COCC(=O)N3)CC1]

### tert-butyl-rel-(6S,7R)-2-oxo-7-({[(1s,4s)-4-(2-ethenylphenyl)cyclohexyl]oxy}methyl)-4-oxa-1,8-diazaspiro[5.5]undecane-8-carboxylate

Intermediate 104 (740 mg), potassium ethenyl(trifluoro)borate(1-) (245 mg, 1.83 mmol) and dicesium carbonate (795 mg, 2.44 mmol) were stirred in 1,4-dioxane (18 mL) and water (3 mL) and degassed with N₂. Bis(triphenylphosphine)palladium (II) dichloride (43 mg, 0.0610 mmol) was added to the reaction solution, which was then further degassed with N₂, sealed, and heated to 80° C. for 16 h. The reaction mixture was concentrated in vacuo. The crude reaction mixture was diluted with EtOAc (30 mL) and water (30 mL), the organic layer was separated and the aqueous layer was extracted with EtOAc (2×20 mL). The combined organic extracts were dried (Na₂SO₄) and filtered. The filtrate was concentrated in vacuo to afford the crude material. The crude material was purified by column chromatography (0-100% EtOAc in heptane) to afford the title compound (606 mg) as a pale-yellow gum. [M+H]⁺ m/z 485.4

### Intermediate 106

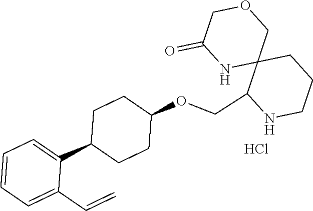 [SMILES: C=Cc1ccccc1[C@H]1CC[C@@H](OCC2NCCCC23COCC(=O)N3)CC1 ; Cl]

### Rel-(6S,7R)-7-({[(1s,4s)-4-(2-ethenylphenyl)cyclohexyl]oxy}methyl)-4-oxa-1,8-diazaspiro[5.5]undecan-2-one hydrochloride

4 M Hydrogen chloride in dioxane (6.0 mL, 24.0 mmol) was added to Intermediate 105 (600 mg) at room temperature and the reaction was stirred for 4 hours. The solvent was removed in vacuo to afford the title compound (504 mg) as a white solid. [M+H]⁺ m/z 385.3

### Intermediate 107

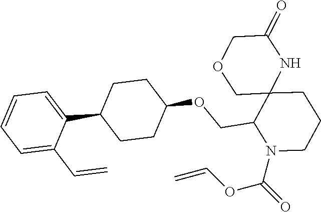 [SMILES: C=COC(=O)N1CCCC2(COCC(=O)N2)C1CO[C@H]1CC[C@@H](c2ccccc2C=C)CC1]

### Ethenyl-rel-(6S,7R)-2-oxo-7-({[(1s,4s)-4-(2-ethenylphenyl)cyclohexyl]oxy}methyl)-4-oxa-1,8-diazaspiro[5.5]undecane-8-carboxylate

Ethenyl carbonochloridate (52 μL, 0.570 mmol) in DCM-Anhydrous (1 mL) was added dropwise to a stirred solution of Intermediate 106 (200 mg) and triethylamine (331 μL, 2.38 mmol) in anhydrous DCM (5 mL) at 0° C. and mixture was stirred for 2 h. Reaction mixture was diluted with DCM then water (15 mL) was added and organic layer separated. The aqueous layer was extracted with DCM (2×20 mL) and the organic fractions combined, dried over sodium sulfate and concentrated under vacuum. The crude material was purified by column chromatography (0-100% EtOAc in Heptane), to afford the title compound (198 mg) as colorless gum. [M+H]⁺ m/z 455.3

### Intermediate 108

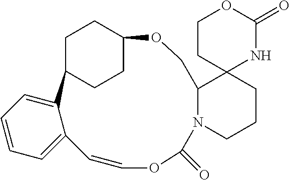 [SMILES: O=C1NC2(CCCN3C(=O)O/C=C\c4ccccc4[C@H]4CC[C@H](CC4)OCC32)CCO1]

### Rel-(1′s,3S,8′Z,17′R,20's)-10′, 19′-dioxa-12′-azaspiro[morpholine-3,16′-tetracyclo[18.2.2.0^{2,7}0.0^{12,17}]tetracosane]-2′,4′,6′,8′-tetraene-5,11′-dione

A solution of Intermediate 107 (150 mg) in anhydrous DCE (60 mL) was degassed with N₂ for 15 mins. [1,3-bis(2,4,6-Trimethylphenyl)imidazolidin-2-ylidene](dichloro)[2-(propan-2-yloxy)benzylidene] ruthenium (62 mg, 0.0990 mmol) was then added and the reaction mixture was heated at 65° C. for 16 hrs. Another 0.3 eq of [1,3-bis(2,4,6-trimethylphenyl)imidazolidin-2-ylidene](dichloro)[2-(propan-2-yloxy)benzylidene] ruthenium was added and the reaction was heated at 65° C. for another 16 hrs. The solvent was removed under reduced pressure. The resulting residue was diluted with DCM then water (15 mL) was added and the organic layer was separated. The aqueous layer was extracted with DCM (2×20 ml) and the organic fractions were combined, dried over sodium sulfate and concentrated under vacuum. The residue was purified by column chromatography (0-100% EtOAc in heptane then 0-20% methanol in DCM) to afford the crude product which was further purified by basic reverse phase column chromatography (10-100% MeCN in Water (0.1% Ammonia)) to afford the title compound (19 mg) as a brown solid. [M+H]⁺ m/z 427.4

### Example 52

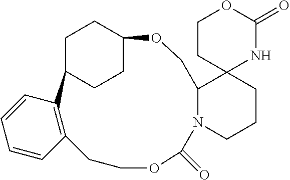 [SMILES: O=C1NC2(CCCN3C(=O)OCCc4ccccc4[C@H]4CC[C@H](CC4)OCC32)CCO1]

### Rel-(1′s,3S,17′R,20's)-10′,19′-dioxa-12′-azaspiro[morpholine-3,16′-tetracyclo [18.2.2.0^{2,7}0.0^{12,17}]tetracosane]-2′(7′), 3′,5′-triene-5,11′-dione

Intermediate 108 (19 mg) was dissolved in ethanol (4 mL) and the atmosphere was evacuated and backfilled with nitrogen 3 times. Palladium on carbon (10%, 9.5 mg, 8.91 μmol) was added and the atmosphere was evacuated and backfilled with hydrogen 3 times. The reaction was stirred for 5 hours and then filtered through Celite, washing with EtOAc and the filtrate was concentrated in vacuo to afford the crude product which was purified by Gilson 9 Gradient Run Methanol/Water (+0.2% Ammonium Hydroxide) 5% to 95% to afford the title compound (1.2 mg) as a white solid.

LCMS (Method B): [M+H]⁺ m/z 429.4, RT 3.19 minutes

¹H NMR (500 MHZ, CDCl₃) δ 7.23-7.08 (m, 4H), 6.24 (s, 1H), 4.66 (dd, J=13.7, 4.8 Hz, 1H), 4.40 (dd, J=7.3, 3.3 Hz, 1H), 4.28 (dd, J=27.5, 16.8 Hz, 1H), 4.18-4.07 (m, 2H), 3.89-3.79 (m, 2H), 3.78-3.70 (m, 1H), 3.66 (s, 1H), 3.45-3.29 (m, 1H), 2.89-2.73 (m, 2H), 2.71-2.62 (m, 2H), 2.29-2.21 (m, 1H), 2.09-1.90 (m, 3H), 1.85-1.65 (m, 5H), 1.27-1.16 (m, 4H).

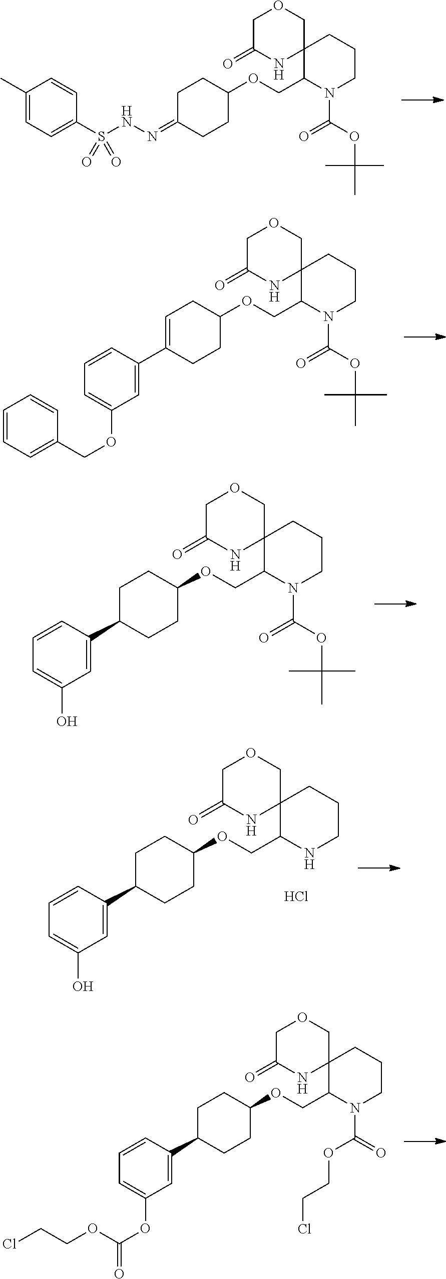 [SMILES: Cc1ccc(S(=O)(=O)NN=C2CCC(OCC3N(C(=O)OC(C)(C)C)CCCC34COCC(=O)N4)CC2)cc1 ; CC(C)(C)OC(=O)N1CCCC2(COCC(=O)N2)C1COC1CC=C(c2cccc(OCc3ccccc3)c2)CC1 ; CC(C)(C)OC(=O)N1CCCC2(COCC(=O)N2)C1CO[C@H]1CC[C@@H](c2cccc(O)c2)CC1 ; O=C1COCC2(CCCNC2CO[C@H]2CC[C@@H](c3cccc(O)c3)CC2)N1 ; Cl ; O=C1COCC2(CCCN(C(=O)OCCCl)C2CO[C@H]2CC[C@@H](c3cccc(OC(=O)OCCCl)c3)CC2)N1]

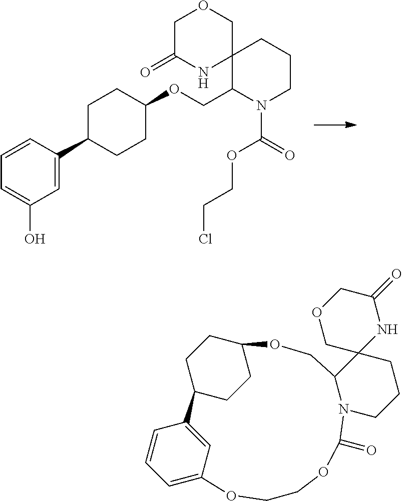 [SMILES: O=C1COCC2(CCCN(C(=O)OCCCl)C2CO[C@H]2CC[C@@H](c3cccc(O)c3)CC2)N1 ; O=C1COCC2(CCCN3C(=O)OCCOc4cccc(c4)[C@H]4CC[C@H](CC4)OCC32)N1]

### Intermediate 109

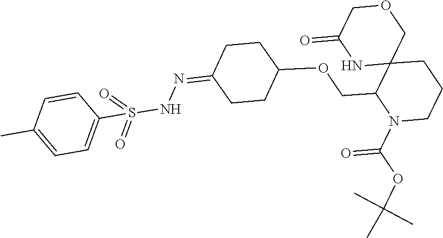 [SMILES: Cc1ccc(S(=O)(=O)NN=C2CCC(OCC3N(C(=O)OC(C)(C)C)CCCC34COCC(=O)N4)CC2)cc1]

### tert-butyl-rel-(6S,7R)-7-[({4-[(4-methylbenzenesulfonamido)imino]cyclohexyl}oxy)methyl]-2-oxo-4-oxa-1,8-diazaspiro[5.5]undecane-8-carboxylate

Intermediate 109 was prepared using known starting material tert-butyl 2-oxo-7-{[(4-oxocyclohexyl)oxy]methyl}-4-oxa-1,8-diazaspiro[5.5]undecane-8-carboxylate following a similar procedure as described for Intermediate 47 to afford the title compound (22.6 g) as a white solid. [M+H]⁺ m/z 565.3.

### Intermediate 110

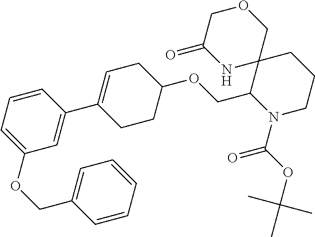 [SMILES: CC(C)(C)OC(=O)N1CCCC2(COCC(=O)N2)C1COC1CC=C(c2cccc(OCc3ccccc3)c2)CC1]

### tert-butyl-rel-(6S, 7R)-7-[({4-[3-(benzyloxy)phenyl]cyclohex-3-en-1-yl}oxy)methyl]-2-oxo-4-oxa-1,8-diazaspiro[5.5]undecane-8-carboxylate

Intermediate 110 was prepared using commercially available starting material 1-iodo-3-(phenoxymethyl)benzene and Intermediate 110 following a similar procedure as described for Intermediate 60 to afford the title compound (1.15 g) as a yellow solid. [M+H]⁺ m/z 563.4

### Intermediate 111

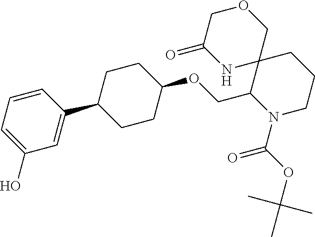 [SMILES: CC(C)(C)OC(=O)N1CCCC2(COCC(=O)N2)C1CO[C@H]1CC[C@@H](c2cccc(O)c2)CC1]

### tert-butyl-rel-(6S,7R)-2-oxo-7-({[(1s,4s)-4-(3-hydroxyphenyl)cyclohexyl]oxy}methyl)-4-oxa-1,8-diazaspiro[5.5]undecane-8-carboxylate

Intermediate 111 was prepared following a similar procedure as described for Intermediate 8 to afford the title compound (952 mg). [M+H]⁺ m/z 475.4

### Intermediate 112

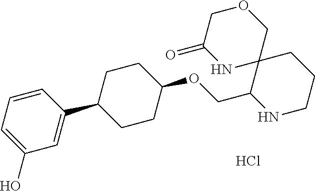 [SMILES: O=C1COCC2(CCCNC2CO[C@H]2CC[C@@H](c3cccc(O)c3)CC2)N1 ; Cl]

### Rel-(6S,7R)-7-({[(1s,4s)-4-(3-hydroxyphenyl)cyclohexyl]oxy}methyl)-4-oxa-1,8-diazaspiro[5.5]undecan-2-one hydrochloride

Intermediate 112 was prepared following a similar procedure as described for Intermediate 12 to afford the title compound (134 mg). [M+H]⁺ m/z 375.2

### Intermediate 113

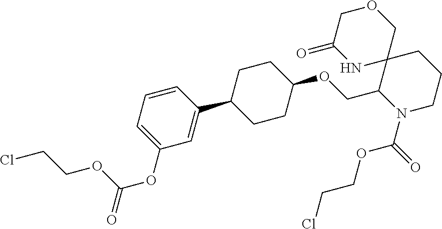 [SMILES: O=C1COCC2(CCCN(C(=O)OCCCl)C2CO[C@H]2CC[C@@H](c3cccc(OC(=O)OCCCl)c3)CC2)N1]

### 2-chloroethyl-rel-(6S,7R)-2-oxo-7-({[(1s,4s)-4-(3-{[(2-chloroethoxy)-carbonyl]oxy}phenyl)cyclohexyl]oxy}methyl)-4-oxa-1,8-diazaspiro[5.5]undecane-8-carboxylate

To a solution of Intermediate 112 (134 mg) in DCM (3.5 mL) was added triethylamine (0.2 mL, 1.43 mmol) followed by carbonochloridic acid 2-chloroethyl ester (0.07 mL, 0.720 mmol). After 30 min at room temperature the reaction was concentrated in vacuo to afford the title compound (275 mg). [M+H]⁺ m/z 587.3

### Intermediate 114

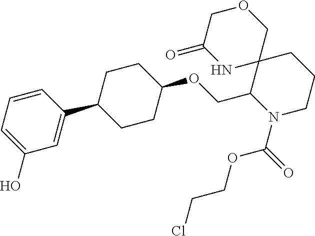 [SMILES: O=C1COCC2(CCCN(C(=O)OCCCl)C2CO[C@H]2CC[C@@H](c3cccc(O)c3)CC2)N1]

### 2-chloroethyl-rel-(6S,7R)-2-oxo-7-({[(1s,4s)-4-(3-hydroxyphenyl)-cyclohexyl]oxy}methyl)-4-oxa-1,8-diazaspiro[5.5]undecane-8-carboxylate

To a solution of Intermediate 113 (253 mg) in methanol (2 mL)/THF (2 mL) was added LiOH (52 mg, 2.15 mmol). After 30 min at room temperature the reaction mixture was acidified with aqueous 2M HCl, then water was added and the crude was extracted with EtOAc. The combined organic fractions were washed with brine, dried over Na₂SO₄, and evaporated in vacuo to afford the title compound (170 mg). [M+H]⁺ m/z 481.3

### Example 53

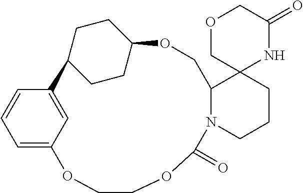 [SMILES: O=C1COCC2(CCCN3C(=O)OCCOc4cccc(c4)[C@H]4CC[C@H](CC4)OCC32)N1]

### Rel-(1′s,3S,17′R,20's)-7′,10′,19′-trioxa-12′-azaspiro[morpholine-3,16′-tetracyclo[18.2.2.1^{2,6}0.0^{12,17}]pentacosane]-2′(25′),3′,5′-triene-5,11′-dione

To a solution of Intermediate 114 (170 mg) in DMF (88 mL) was added cesium carbonate (691 mg, 2.12 mmol) and the reaction mixture was stirred at °60 C for 15 h. The reaction mixture was filtered and the filtrate was concentrated in vacuo. The crude product was purified by a first reverse phase column chromatography (0-100% acetonitrile+0.1% formic acid in H₂O+0.1% formic acid) followed by a second column chromatography (30-60% EtOAc+20% EtOH in cyclohexane) to afford the title compound (17.5 mg).

LCMS (Method C): [M+H]⁺ m/z 445.3, RT 0.99 minutes

¹H NMR (500 MHZ, CDCl₃) δ 7.11 (br t, J=7.8 Hz, 1H), 6.85 (br s, 1H), 6.70-6.78 (m, 2H), 5.85 (br s, 1H), 5.00-5.17 (m, 1H), 4.79 (br d, J=12.8 Hz, 1H), 4.64 (br dd, J=14.1, 10.0 Hz, 1H), 4.32 (br d, J=17.0 Hz, 1H), 4.27 (br d, J=14.4 Hz, 1H), 4.07-4.21 (m, 4H), 3.85 (br t, J=9.7 Hz, 1H), 3.74 (br s, 1H), 3.65 (br dd, J=8.9, 3.1 Hz, 1H), 3.35 (br d, J=11.8 Hz, 1H), 2.92-3.05 (m, 1H), 2.50-2.64 (m, 1H), 1.91-2.12 (m, 4H), 1.36-1.87 (m, 8H).

### Examples 53a and 53b

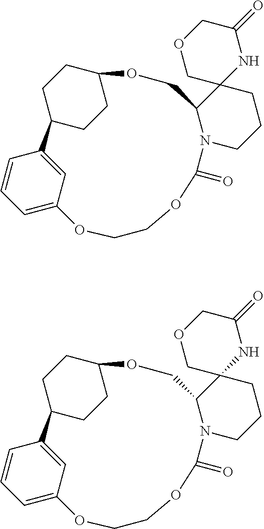 [SMILES: O=C1COCC2(CCCN3C(=O)OCCOc4cccc(c4)[C@H]4CC[C@H](CC4)OC[C@H]32)N1 ; O=C1COC[C@]2(CCCN3C(=O)OCCOc4cccc(c4)[C@H]4CC[C@H](CC4)OC[C@@H]32)N1]

### Example 53a: (1′r,3R,17′S,20′r)-7′,10′,19′-trioxa-12′-azaspiro[morpholine-3,16′-tetracyclo[18.2.2.1^{2,6}0.0^{12,17}] pentacosane]-2′(25′),3′,5′-triene-5,11′-dione

### Example 53b: (1′s,3R,17'S,20's)-7′,10′,19′-trioxa-12′-azaspiro[morpholine-3,16′-tetracyclo[18.2.2.1^{2,6}0.0^{12,17}] pentacosane]-2′(25′),3′,5′-triene-5,11′-dione

Example 53 (15.4 mg) was subjected to chiral preparative purification using Waters 600 eluting with 50/50% v/v n-Hexane/ethanol+0.1% isopropylamine, Chiralpak AS-H (25×2.0 cm), 5 μm, flow rate 17 mL/minute to afford the title compounds (Peak 1, 4.18 mg, 100% ee; and Peak 2, 4.61 mg, 100% ee).

### Example 53a: Peak 1 (Stereochemistry Tentatively Assigned)

LCMS (Method C): [M+H]⁺ m/z 445.3, RT 0.99 minutes.

Chiral analysis (Chiralpak AS-H, 25×0.46 cm, 5 μm, 50:50 n-Hexane: ethanol+0.1% isopropylamine): RT 6.2 minutes

¹H NMR (400 MHZ, CDCl₃) δ 7.11 (br t, J=7.8 Hz, 1H), 6.85 (br s, 1H), 6.70-6.78 (m, 2H), 5.85 (br s, 1H), 5.00-5.17 (m, 1H), 4.79 (br d, J=12.8 Hz, 1H), 4.64 (br dd, J=14.1, 10.0 Hz, 1H), 4.32 (br d, J=17.0 Hz, 1H), 4.27 (br d, J=14.4 Hz, 1H), 4.07-4.21 (m, 4H), 3.85 (br t, J=9.7 Hz, 1H), 3.74 (br s, 1H), 3.65 (br dd, J=8.9, 3.1 Hz, 1H), 3.35 (br d, J=11.8 Hz, 1H), 2.92-3.05 (m, 1H), 2.50-2.64 (m, 1H), 1.91-2.12 (m, 4H), 1.36-1.87 (m, 8H).

### Example 53b: Peak 2 (Stereochemistry Tentatively Assigned)

LCMS (Method C): [M+H]⁺ m/z 445.3, RT 0.99 minutes.

Chiral analysis (Chiralpak AS-H, 25×0.46 cm, 5 μm, 50:50 n-Hexane: ethanol+0.1% isopropylamine): RT 10.2 minutes

¹H NMR (400 MHZ, CDCl₃) δ 7.11 (br t, J=7.8 Hz, 1H), 6.85 (br s, 1H), 6.70-6.78 (m, 2H), 5.85 (br s, 1H), 5.00-5.17 (m, 1H), 4.79 (br d, J=12.8 Hz, 1H), 4.64 (br dd, J=14.1, 10.0 Hz, 1H), 4.32 (br d, J=17.0 Hz, 1H), 4.27 (br d, J=14.4 Hz, 1H), 4.07-4.21 (m, 4H), 3.85 (br t, J=9.7 Hz, 1H), 3.74 (br s, 1H), 3.65 (br dd, J=8.9, 3.1 Hz, 1H), 3.35 (br d, J=11.8 Hz, 1H), 2.92-3.05 (m, 1H), 2.50-2.64 (m, 1H), 1.91-2.12 (m, 4H), 1.36-1.87 (m, 8H).

### Example 54

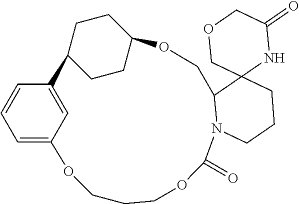 [SMILES: O=C1COCC2(CCCN3C(=O)OCCCOc4cccc(c4)[C@H]4CC[C@H](CC4)OCC32)N1]

### Rel-(1′s,3S,18′R,21′s)-7′,11′,20′-trioxa-13′-azaspiro[morpholine-3,17′-tetracyclo[19.2.2.1^{2,6}0.0^{13,18}] hexacosane]-2′(26′),3′,5′-triene-5,12′-dione

Example 54 was prepared using Intermediate 113 following a similar procedure as described for Example 53 to afford the title compound (10.9 mg).

LCMS (Method C): [M+H]⁺ m/z 459.3, RT 1.01 minutes

¹H NMR (400 MHZ, CDCl₃) δ 7.12 (t, J=7.7 Hz, 1H), 6.81-6.68 (m, 3H), 6.30 (br s, 1H), 4.96-4.85 (m, 1H), 4.78-4.68 (m, 1H), 4.45-4.34 (m, 1H), 4.33-4.22 (m, 2H), 4.21-4.06 (m, 3H), 4.00-3.90 (m, 1H), 3.87-3.78 (m, 1H), 3.76-3.67 (m, 2H), 3.35 (br d, J=11.7 Hz, 1H), 3.30-3.16 (m, 1H), 2.60-2.47 (m, 1H), 2.23 (td, J=14.1, 7.8 Hz, 1H), 1.96-1.89 (m, 1H), 2.14-1.38 (m, 12H).

### Example 54a and 54b

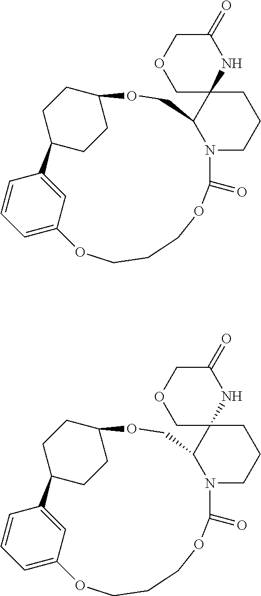 [SMILES: O=C1COC[C@@]2(CCCN3C(=O)OCCCOc4cccc(c4)[C@H]4CC[C@H](CC4)OC[C@H]32)N1 ; O=C1COC[C@]2(CCCN3C(=O)OCCCOc4cccc(c4)[C@H]4CC[C@H](CC4)OC[C@@H]32)N1]

### Example 54a: (1′s,3S,18′R,21′s)-7′,11′,20′-trioxa-13′-azaspiro[morpholine-3,17′-tetracyclo[19.2.2.1^{2,6}0.0^{13,18}] hexacosane]-2′(26′),3′,5′-triene-5,12′-dione

### Example 54b: (1′s,3R,18′S,21′s)-7′,11′,20′-trioxa-13′-azaspiro[morpholine-3,17′-tetracyclo[19.2.2.1^{2,6}0.0^{13,18}] hexacosane]-2′(26′),3′,5′-triene-5,12′-dione

Example 54 (9.3 mg) was subjected to chiral preparative purification using Waters 600 eluting with 50/50% v/v n-Hexane/ethanol+0.1% isopropylamine, Chiralpak IC (25×2.0 cm), 5 μm, flow rate 17 mL/minute to afford the title compounds (Peak 1, 1.23 mg, 100% ee; and Peak 2, 1.16 mg, 100% ee).

### Example 54a: Peak 1 (Stereochemistry Tentatively Assigned)

LCMS (Method C): [M+H]⁺ m/z 459.3, RT 1.02 minutes.

Chiral analysis (Chiralpak IC, 25×0.46 cm, 5 μm, 50:50 n-Hexane: ethanol+0.1% isopropylamine): RT 6.7 minutes

¹H NMR (400 MHZ, CDCl₃) δ 7.12 (t, J=7.7 Hz, 1H), 6.63-7.03 (m, 3H), 6.13 (s, 1H), 4.90 (dd, J=7.8, 3.6 Hz, 1H), 4.57-4.79 (m, 1H), 4.34-4.46 (m, 1H), 4.25-4.32 (m, 1H), 4.22-4.29 (m, 1H), 4.15-4.21 (m, 1H), 4.13-4.18 (m, 1H), 4.06-4.13 (m, 1H), 3.89-4.02 (m, 1H), 3.79-3.86 (m, 1H), 3.69-3.77 (m, 2H), 3.35 (d, J=11.7 Hz, 1H), 3.25 (td, J=13.1, 3.0 Hz, 1 H), 2.53 (tt, J=12.1, 3.5 Hz, 1H), 2.18-2.35 (m, 1H), 1.86-1.98 (m, 1H), 1.37-2.15 (m, 12H).

### Example 54b: Peak 2 (Stereochemistry Tentatively Assigned)

LCMS (Method C): [M+H]⁺ m/z 459.3, RT 1.02 minutes.

Chiral analysis (Chiralpak IC, 25×0.46 cm, 5 μm, 50:50 n-Hexane: ethanol+0.1% isopropylamine): RT 8.6 minutes

¹H NMR (400 MHZ, CDCl₃) δ 7.12 (t, J=7.7 Hz, 1H), 6.63-7.03 (m, 3H), 6.13 (s, 1H), 4.90 (dd, J=7.8, 3.6 Hz, 1H), 4.57-4.79 (m, 1H), 4.34-4.46 (m, 1H), 4.25-4.32 (m, 1H), 4.22-4.29 (m, 1H), 4.15-4.21 (m, 1H), 4.13-4.18 (m, 1H), 4.06-4.13 (m, 1H), 3.89-4.02 (m, 1H), 3.79-3.86 (m, 1H), 3.69-3.77 (m, 2H), 3.35 (d, J=11.7 Hz, 1H), 3.25 (td, J=13.1, 3.0 Hz, 1 H), 2.53 (tt, J=12.1, 3.5 Hz, 1H), 2.18-2.35 (m, 1H), 1.86-1.98 (m, 1H), 1.37-2.15 (m, 12H).

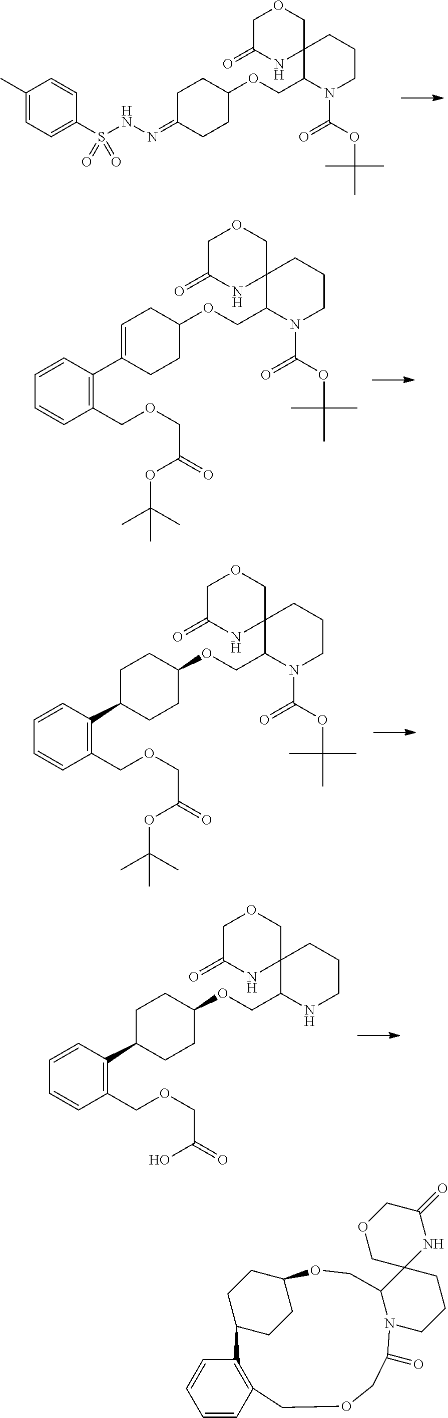 [SMILES: Cc1ccc(S(=O)(=O)NN=C2CCC(OCC3N(C(=O)OC(C)(C)C)CCCC34COCC(=O)N4)CC2)cc1 ; CC(C)(C)OC(=O)COCc1ccccc1C1=CCC(OCC2N(C(=O)OC(C)(C)C)CCCC23COCC(=O)N3)CC1 ; CC(C)(C)OC(=O)COCc1ccccc1[C@H]1CC[C@@H](OCC2N(C(=O)OC(C)(C)C)CCCC23COCC(=O)N3)CC1 ; O=C(O)COCc1ccccc1[C@H]1CC[C@@H](OCC2NCCCC23COCC(=O)N3)CC1 ; O=C1COCC2(CCCN3C(=O)COCc4ccccc4[C@H]4CC[C@H](CC4)OCC32)N1]

### Intermediate 115

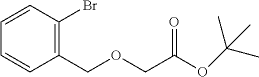 [SMILES: CC(C)(C)OC(=O)COCc1ccccc1Br]

### tert-butyl 2-[(2-bromophenyl)methoxy]acetate

To a mixture of (2-bromophenyl)methanol (1.0 g, 5.35 mmol) and sodium hydride (60%, 134 mg, 5.61 mmol) in THF (27 mL), was added 2-bromoacetic acid tert-butyl ester (0.95 mL, 6.42 mmol). The mixture was stirred at room temperature for 16 h. The mixture was diluted with EtOAc and washed with water. The combined organic extracts were dried (Na₂SO₄) and evaporated in vacuo. The crude product was purified by column chromatography (0-10% EtOAc in cyclohexane) to afford the title compound (1.76 g) as a colorless oil. [M+H+Na]⁺m/z 323.1, 325.1

### Intermediate 116

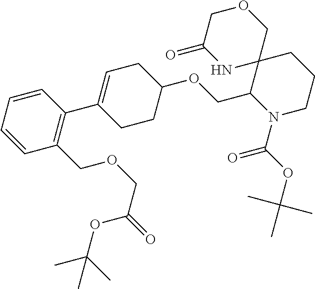 [SMILES: CC(C)(C)OC(=O)COCc1ccccc1C1=CCC(OCC2N(C(=O)OC(C)(C)C)CCCC23COCC(=O)N3)CC1]

### tert-butyl-rel-(6S,7R)-7-({[4-(2-{[2-(tert-butoxy)-2-oxoethoxy]methyl}phenyl)cyclohex-3-en-1-yl]oxy}methyl)-2-oxo-4-oxa-1,8-diazaspiro[5.5]undecane-8-carboxylate

A solution of Intermediate 109 (300 mg), cesium carbonate (432 mg, 1.33 mmol), Intermediate 115 (200.01 mg) and cataCXium® (19 mg, 0.050 mmol) in 1,4-dioxane (3 mL) was evacuated and backfilled with nitrogen three times. Then Pd(OAc)₂ (6 mg, 0.030 mmol) was added and the mixture was heated to 100° C. under a nitrogen atmosphere for 16 hours. The reaction mixture was cooled and filtered through a Celite pad washing with DCM. The filtrate was concentrated in vacuo and then diluted with DCM (50 ml). The organic phase was washed with water (50 mL) and brine (50 mL), dried over Na₂SO₄, filtered and concentrated in vacuo. The product was purified by column chromatography (0-40% EtOAc/EtOH 9:1 in cyclohexane) to afford the title compound (217 mg) as a yellow oil. [M+H]⁺ m/z 601.4

### Intermediate 117

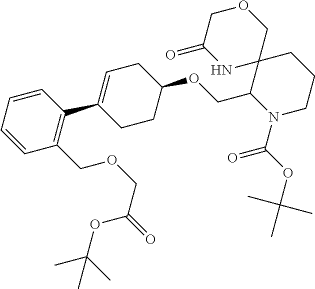

### tert-butyl-rel-(6S,7R)-2-oxo-7-({[(1s,4s)-4-(2-{[2-(tert-butoxy)-2-oxoethoxy]methyl}phenyl)cyclohexyl]oxy}methyl)-4-oxa-1,8-diazaspiro[5.5]undecane-8-carboxylate

To a solution of Intermediate 116 (197 mg) in ethanol (22 mL), sodium carbonate (104 mg, 0.980 mmol) and palladium on carbon (10%, 69 mg, 0.070 mmol) were added. The mixture was set under a hydrogen atmosphere (1 atm.) and stirred for 40 min. The mixture was filtered over a Celite pad and the filtrate was concentrated in vacuo. The crude was taken up with EtOAc and the solution was washed with water (50 mL), brine (50 mL), dried over anhydrous Na₂SO₄ and concentrated in vacuo to afford the title compound (177 mg) as a white solid. [M+H]⁺ m/z 603.4

### Intermediate 118

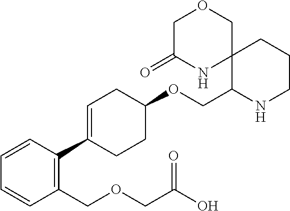

### 2-({2-[(1s,4s)-4-{[rel-(6S,7R)-2-oxo-4-oxa-1,8-diazaspiro[5.5]undecan-7-yl]methoxy}cyclohexyl]phenyl}methoxy)acetic acid

Intermediate 118 was prepared following a similar procedure as described for Intermediate 32 to afford the title compound (22 mg) as a white solid. [M+H]⁺ m/z 447.3

### Example 55

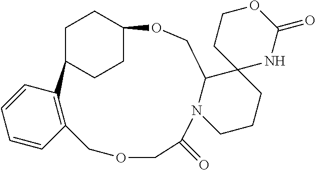 [SMILES: O=C1NC2(CCCN3C(=O)COCc4ccccc4[C@H]4CC[C@H](CC4)OCC32)CCO1]

### Rel-(1′s,3S,17′R,20's)-9′,19′-dioxa-12′-azaspiro[morpholine-3,16′-tetracyclo[18.2.2.0^{2,7}0.0^{12,17}]tetracosane]-2′(7′),3′,5′-triene-5,11′-dione

Example 55 was prepared using Intermediate 118 following a similar procedure as described for Example 8 to afford the title compound (3 mg) as a white solid.

LCMS (Method C): [M+H]⁺ m/z 429.5, RT 0.87 min

¹H NMR (400 MHZ, CDCl₃) δ 7.36-7.43 (m, 1H), 7.09-7.25 (m, 3H), 6.20 (br s, 1H), 5.51 (dd, J=11.9, 3.7 Hz, 1H), 5.25 (d, J=9.9 Hz, 1H), 4.72 (d, J=12.7 Hz, 1H), 4.26-4.37 (m, 1H), 4.23 (d, J=10.0 Hz, 1H), 4.07-4.19 (m, 2H), 3.95-4.05 (m, 2H), 3.89 (br dd, J=11.8, 9.2 Hz, 1H), 3.68-3.75 (m, 1H), 3.65 (br dd, J=9.0, 4.1 Hz, 1H), 3.32-3.41 (m, 1H), 3.11-3.26 (m, 1H), 2.60-2.78 (m, 1H), 1.34-2.26 (m, 12H).

### Example 56

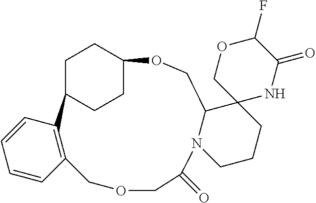 [SMILES: O=C1NC2(CCCN3C(=O)COCc4ccccc4[C@H]4CC[C@H](CC4)OCC32)COC1F]

### Rel-(1′s,3S,16′R,19′s)-6-fluoro-8′,18′-dioxa-11′-azaspiro[morpholine-3,15′-tetracyclo[17.2.2.0^{2,7}0.0^{11,16}]tricosane]-2′(7′),3′,5′-triene-5,10′-dione

Example 56 was prepared using commercially available starting material 1-tert-butyl 4-ethyl 3-oxopiperidine-1,4-dicarboxylate following a similar procedure as described for Example 8 to afford the title compound (36 mg) as a white solid. This was subjected to achiral preparative purification using UPLC Waters with mass spectrometry detection (MS:SQD2). Column: CSH C18 (2.1×50 mm, 1.7 μm). Conditions: [A1: Waters+0.1% HCOOH]; [B1: MeCN+0.1% HCOOH]. Gradient: from 3% B1 to 99.9% B1 in 1.4 min (flow: 0.90 mL/min). Detection: UV/Vis detection range 210 nm to 350 nm MS (ES+/ES−). Scan range 100 to 1000 AMU to afford the title compounds (10.7 mg).

LCMS (Method B): [M+H]⁺ m/z 433.3, RT 2.99

¹H NMR (400 MHZ, CDCl₃) δ 7.16 (td, J=7.9, 1.7 Hz, 1H), 7.09 (dd, J=7.4, 1.6 Hz, 1H), 6.90 (td, J=7.4, 1.0 Hz, 1H), 6.78-6.72 (m, 1H), 6.70 (s, 1H), 5.58 (d, J=52.0 Hz, 1H), 5.42 (dd, J=10.4, 4.0 Hz, 1H), 5.11 (d, J=10.6 Hz, 1H), 4.33 (d, J=10.6 Hz, 1H), 4.17 (d, J=11.4 Hz, 1H), 3.91-3.83 (m, 2H), 3.79-3.67 (m, 2H), 3.62-3.46 (m, 2H), 2.70-2.50 (m, 2H), 2.32-2.17 (m, 1H), 2.13-2.01 (m, 1H), 1.94-1.74 (m, 3H), 1.55-1.31 (m, 6H).

### Examples 56a and 56b

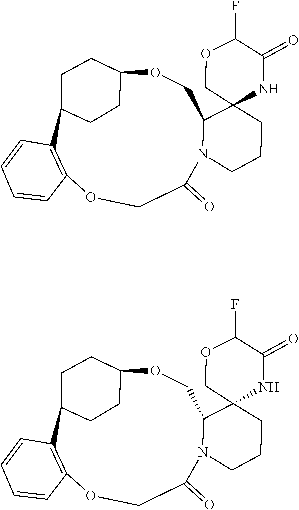

### Example 56a: (1′s,3S,16′R,19′s)-6-fluoro-8′,18′-dioxa-11′-azaspiro[morpholine-3,15′-tetracyclo[17.2.2.0^{2,7}0.0^{11,16}]tricosane]-2′(7′),3′,5′-triene-5,10′-dione

### Example 56b: (1′s,3R,16′S,19′s)-6-fluoro-8′,18′-dioxa-11′-azaspiro[morpholine-3,15′-tetracyclo[17.2.2.0^{2,7}0.0^{11,16}]tricosane]-2′(7′),3′,5′-triene-5,10′-dione

Example 56 (36 mg, 0.083 mmol) was subjected to chiral preparative purification using Waters 600 and Chiralpak IC, 25×2.0 cm, 5 μm column eluting with 55:45 Heptane: Ethanol+0.1% isopropylamine to afford the title compounds (Peak 1, 2.84 mg, 100% ee; and Peak 2, 3.4 mg, 97.7% ee).

### Example 56a: Peak 1 (Stereochemistry Tentatively Assigned)

LCMS (Method C): [M+H]⁺ m/z 433.3, RT 0.99 min

Chiral analysis (Chiralpak IC, 25×0.46 cm, 5 μm, 55:45 n-Hexane: Ethanol+0.1% isopropylamine: RT 6.3 minutes

¹H NMR (500 MHZ, CDCl₃) δ 7.18 (td, J=7.7, 1.7 Hz, 1H), 7.10 (dd, J=7.4, 1.5 Hz, 1H), 6.91 (td, J=7.4, 1.0 Hz, 1H), 6.76 (d, J=8.0 Hz, 1H), 5.91 (br s, 1H), 5.61 (d, J=52.0 Hz, 1H), 5.43 (dd, J=10.1, 3.8 Hz, 1H), 5.12 (d, J=10.6 Hz, 1H), 4.34 (d, J=10.6 Hz, 1H), 4.18 (d, J=11.7 Hz, 1H), 3.91-3.83 (m, 2H), 3.80-3.71 (m, 2H), 3.60-3.51 (m, 2H), 2.71-2.52 (m, 2H), 2.29-2.19 (m, 1H), 2.09-2.02 (m, 1H), 1.95-1.88 (m, 1H), 1.89-1.80 (m, 2H), 1.80-1.74 (m, 1H), 1.56 (s, 2H), 1.47-1.33 (m, 3H).

### Example 56b: Peak 2 (Stereochemistry Tentatively Assigned)

LCMS (Method C): [M+H]⁺ m/z 433.3, RT 0.99 min

Chiral analysis (Chiralpak IC, 25×0.46 cm, 5 μm, 55:45 n-Hexane: Ethanol+0.1% isopropylamine: RT 7.5 minutes

¹H NMR (500 MHZ, CDCl₃) δ 7.18 (td, J=7.7, 1.7 Hz, 1H), 7.10 (dd, J=7.4, 1.5 Hz, 1H), 6.91 (td, J=7.4, 1.0 Hz, 1H), 6.76 (d, J=8.0 Hz, 1H), 5.91 (br s, 1H), 5.61 (d, J=52.0 Hz, 1H), 5.43 (dd, J=10.1, 3.8 Hz, 1H), 5.12 (d, J=10.6 Hz, 1H), 4.34 (d, J=10.6 Hz, 1H), 4.18 (d, J=11.7 Hz, 1H), 3.91-3.83 (m, 2H), 3.80-3.71 (m, 2H), 3.60-3.51 (m, 2H), 2.71-2.52 (m, 2H), 2.29-2.19 (m, 1H), 2.09-2.02 (m, 1H), 1.95-1.88 (m, 1H), 1.89-1.80 (m, 2H), 1.80-1.74 (m, 1H), 1.56 (s, 2H), 1.47-1.33 (m, 3H).

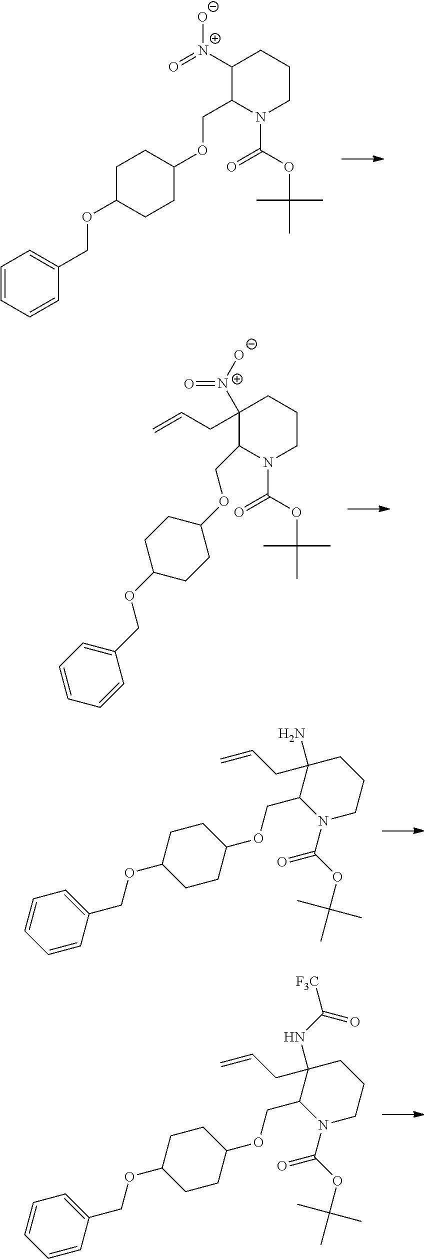

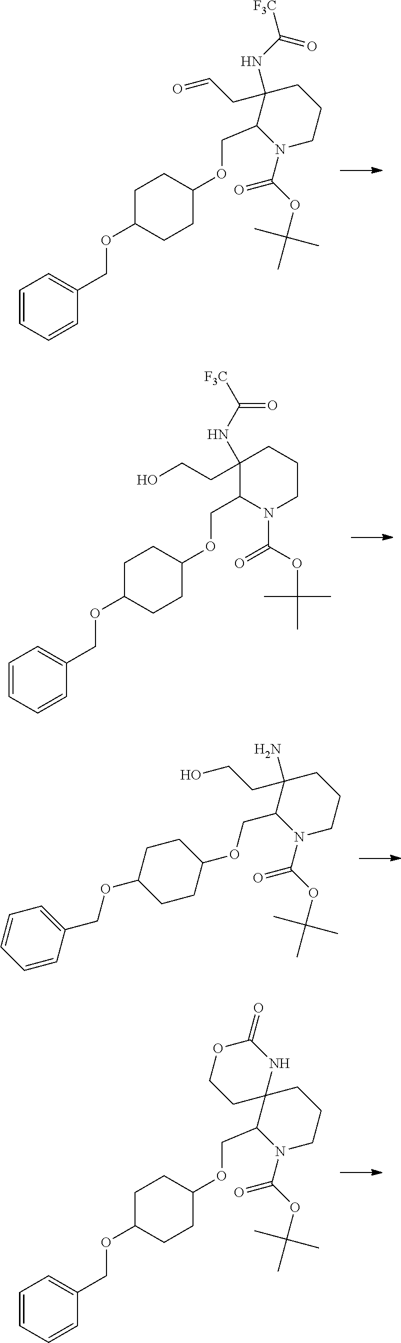 [SMILES: CC(C)(C)OC(=O)N1CCCC(CC=O)(NC(=O)C(F)(F)F)C1COC1CCC(OCc2ccccc2)CC1 ; CC(C)(C)OC(=O)N1CCCC(CCO)(NC(=O)C(F)(F)F)C1COC1CCC(OCc2ccccc2)CC1 ; CC(C)(C)OC(=O)N1CCCC(N)(CCO)C1COC1CCC(OCc2ccccc2)CC1 ; CC(C)(C)OC(=O)N1CCCC2(CCOC(=O)N2)C1COC1CCC(OCc2ccccc2)CC1]

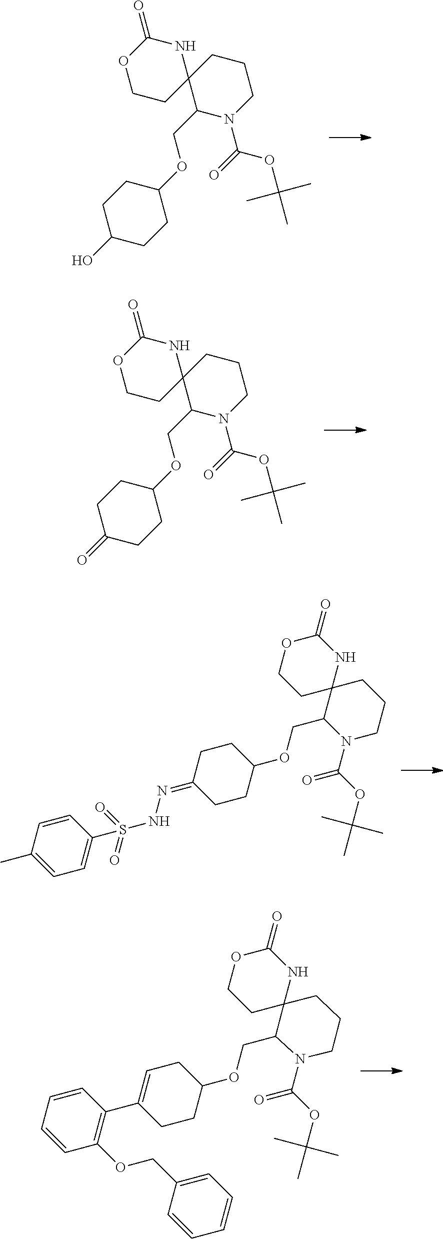 [SMILES: CC(C)(C)OC(=O)N1CCCC2(CCOC(=O)N2)C1COC1CCC(O)CC1 ; CC(C)(C)OC(=O)N1CCCC2(CCOC(=O)N2)C1COC1CCC(=O)CC1 ; Cc1ccc(S(=O)(=O)NN=C2CCC(OCC3N(C(=O)OC(C)(C)C)CCCC34CCOC(=O)N4)CC2)cc1 ; CC(C)(C)OC(=O)N1CCCC2(CCOC(=O)N2)C1COC1CC=C(c2ccccc2OCc2ccccc2)CC1]

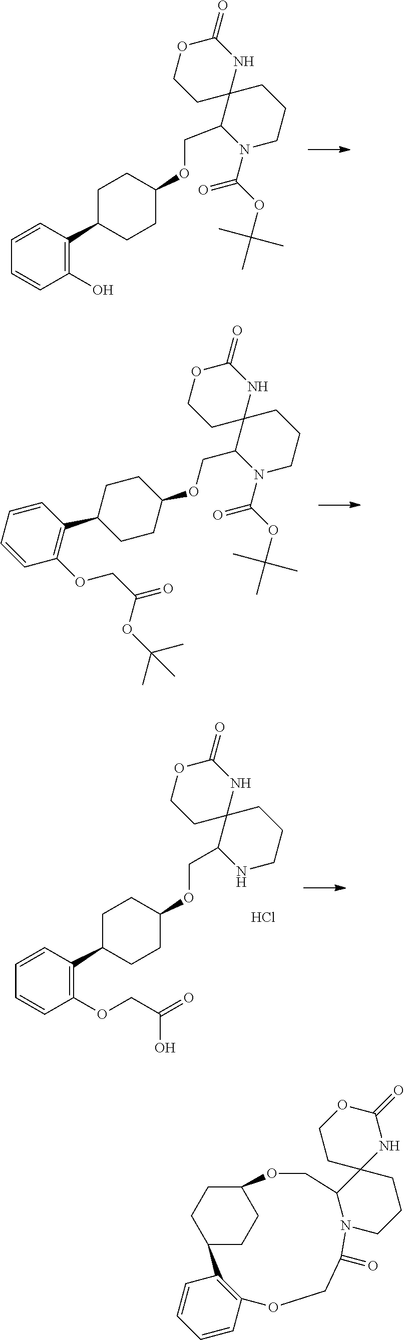 [SMILES: CC(C)(C)OC(=O)N1CCCC2(CCOC(=O)N2)C1CO[C@H]1CC[C@@H](c2ccccc2O)CC1 ; CC(C)(C)OC(=O)COc1ccccc1[C@H]1CC[C@@H](OCC2N(C(=O)OC(C)(C)C)CCCC23CCOC(=O)N3)CC1 ; O=C(O)COc1ccccc1[C@H]1CC[C@@H](OCC2NCCCC23CCOC(=O)N3)CC1 ; Cl ; O=C1NC2(CCCN3C(=O)COc4ccccc4[C@H]4CC[C@H](CC4)OCC32)CCO1]

### Intermediate 119

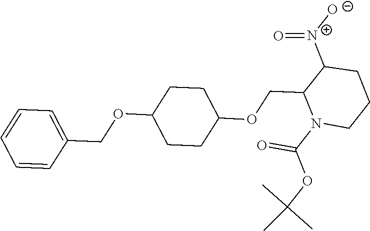 [SMILES: CC(C)(C)OC(=O)N1CCCC([N+](=O)[O-])C1COC1CCC(OCc2ccccc2)CC1]

### tert-butyl 2-({[4-(benzyloxy)cyclohexyl]oxy}methyl)-3-nitropiperidine-1-carboxylate

Intermediate 119 was prepared using commercially available starting material 1-tert-butyl 4-ethyl 3-oxopiperidine-1,4-dicarboxylate following a similar procedure as described for Intermediate 41 to afford the title compound (12.2 g) as a yellow oil. [M-Boc+H]⁺ m/z 349.3

### Intermediate 120

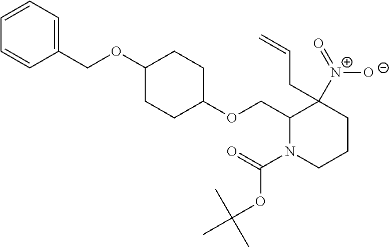 [SMILES: C=CCC1([N+](=O)[O-])CCCN(C(=O)OC(C)(C)C)C1COC1CCC(OCc2ccccc2)CC1]

### tert-butyl-rel-(2R,3R)-2-({[4-(benzyloxy)cyclohexyl]oxy}methyl)-3-nitro-3-(prop-2-en-1-yl)piperidine-1-carboxylate

A solution of potassium hydroxide (1.17 g, 20.87 mmol) in 2-propanol (42 mL) and methanol (42 mL) was stirred for 1 h. A solution of Intermediate 119 (8.51 g) in methanol (42 mL) was added. The solution was then degassed and Pd(OAc)₂ (426 mg, 1.9 mmol) was added followed by triphenylphosphine (746 mg, 2.85 mmol). The solution was heated to 45° C. 5 minutes before allyl acetate (2.25 mL, 20.87 mmol) was added. The reaction mixture was heated at 55° C. under nitrogen for 16 h then cooled to room temperature. The mixture was concentrated in vacuo and re-dissolved in MTBE. The mixture was filtered over a pad of Celite and washed with water. The organic extracts were dried (Na₂SO₄) and concentrated in vacuo. The crude product was purified by column chromatography (0-10% EtOAc in cyclohexane) to afford the title compound (5.8 g) as a colorless oil. [M+H]⁺ m/z 489.4

### Intermediate 121

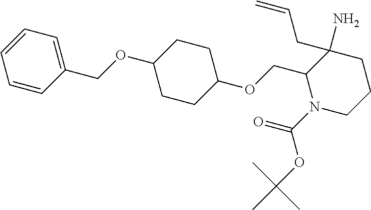 [SMILES: C=CCC1(N)CCCN(C(=O)OC(C)(C)C)C1COC1CCC(OCc2ccccc2)CC1]

### tert-butyl-rel-(2R,3R)-3-amino-2-({[4-(benzyloxy)cyclohexyl]oxy}methyl)-3-(prop-2-en-1-yl)piperidine-1-carboxylate

Intermediate 121 was prepared following a similar procedure as described for Intermediate 6 to afford the title compound (3.85 g) as a yellow oil. [M+H]⁺ m/z 459.4

### Intermediate 122

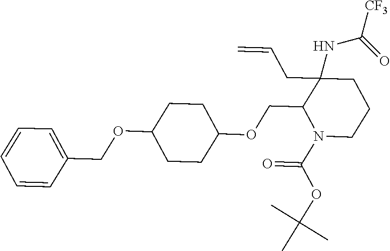 [SMILES: C=CCC1(NC(=O)C(F)(F)F)CCCN(C(=O)OC(C)(C)C)C1COC1CCC(OCc2ccccc2)CC1]

### tert-butyl-rel-(2R,3R)-2-({[4-(benzyloxy)cyclohexyl]oxy}methyl)-3-(prop-2-en-1-yl)-3-(2,2,2-trifluoro acetamido)piperidine-1-carboxylate

To a solution of Intermediate 121 (300 mg) in DCM (6.5 mL), was added triethylamine (0.18 mL, 1.31 mmol) and trifluoroacetic anhydride (0.1 mL, 0.690 mmol) dropwise at 0° C. The reaction mixture was stirred for 1 h, then it was diluted with DCM and washed with water, sat. aq. NaHCO₃ and aq. 1M Na₂CO₃ until pH=9. The organic layer was dried (Na₂SO₄) and concentrated in vacuo to afford the title compound (337 mg) as a yellow oil. [M+H]⁺ m/z 555.5

### Intermediate 123

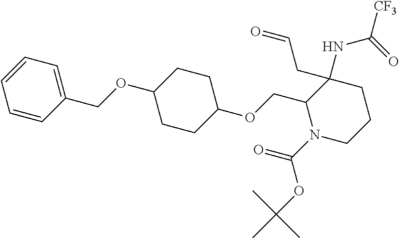 [SMILES: CC(C)(C)OC(=O)N1CCCC(CC=O)(NC(=O)C(F)(F)F)C1COC1CCC(OCc2ccccc2)CC1]

### tert-butyl-rel-(2R,3R)-2-({[4-(benzyloxy)cyclohexyl]oxy}methyl)-3-(2-oxoethyl)-3-(2,2,2-trifluoroacetamido)piperidine-1-carboxylate

In a 3-necked round bottom flask Intermediate 122 (3.13 g) was dissolved in DCM (256 mL) and the solution was stirred at −78° C. while ozone was bubbled through the solution. After 15 min the ozone was replaced by N₂. After 10 min triphenylphosphine (1.48 g, 5.64 mmol) in DCM (9 mL) was slowly added to the cold mixture. The mixture was allowed to slowly warm to room temperature and concentrated in vacuo to afford a transparent oil. The crude product was purified by column chromatography (0-30% EtOAc+EtOH 10% in cyclohexane) to afford the title compound (2 g). [M-Boc+H]⁺ m/z 457.5

### Intermediate 124

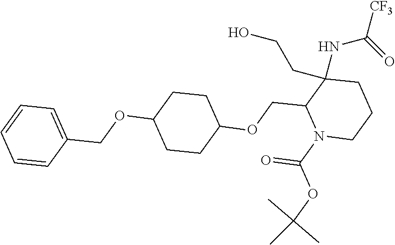 [SMILES: CC(C)(C)OC(=O)N1CCCC(CCO)(NC(=O)C(F)(F)F)C1COC1CCC(OCc2ccccc2)CC1]

### tert-butyl-rel-(2R,3R)-2-({[4-(benzyloxy)cyclohexyl]oxy}methyl)-3-(2-hydroxyethyl)-3-(2,2,2-trifluoro acetamido)piperidine-1-carboxylate

To a mixture of Intermediate 123 (2.0 g) and methanol (17 mL) at 0° C., was added sodium borohydride (136 mg, 3.59 mmol). The suspension was stirred at 0° C. for 1 h, then the reaction was warmed to room temperature and the mixture was diluted with EtOAc and washed with water (100 mL). The aqueous phase was extracted three times with EtOAc and the combined organic phases were washed with brine, dried over Na₂SO₄ and concentrated in vacuo to afford the title compound (2 g). [M-Boc+H]⁺ m/z 459.3

### Intermediate 125

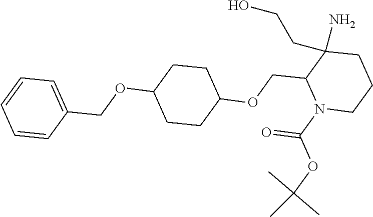 [SMILES: CC(C)(C)OC(=O)N1CCCC(N)(CCO)C1COC1CCC(OCc2ccccc2)CC1]

### tert-butyl-rel-(2R,3R)-3-amino-2-({[4-(benzyloxy)cyclohexyl]oxy}methyl)-3-(2-hydroxyethyl)piperidine-1-carboxylate

To a solution of Intermediate 124 (2.01 g) in methanol (9 mL) and water (4.5 mL), potassium carbonate (2.49 g, 17.99 mmol) was added. The mixture was stirred at 50° C. for 1h, then the mixture was diluted with EtOAc and washed with H₂O (50 mL) and brine (50 mL), dried over Na₂SO₄ and concentrated in vacuo to afford the title compound (1.51 g). [M+H]⁺ m/z 463.4

### Intermediate 126

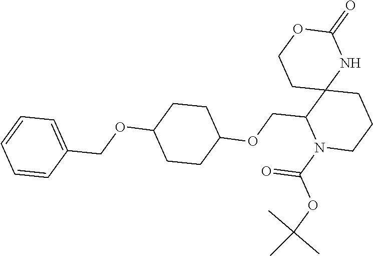 [SMILES: CC(C)(C)OC(=O)N1CCCC2(CCOC(=O)N2)C1COC1CCC(OCc2ccccc2)CC1]

### tert-butyl-rel-(6R, 7R)-7-({[4-(benzyloxy)cyclohexyl]oxy}methyl)-2-oxo-3-oxa-1,8-diazaspiro[5.5]undecane-8-carboxylate

A solution of N,N-diisopropylethylamine (0.85 mL, 4.86 mmol) was added dropwise to a stirred solution of carbonic acid bis(trichloromethyl) ester (1.85 g, 6.24 mmol) and Intermediate 125 (1.5 g) in anhydrous DCM (58 mL) at 0° C. and the resulting mixture was stirred for 30 mins. The reaction mixture was warmed to room temperature and purged for 30 mins with a stream of N₂ until pH=9 (the exhaust gasses were passed through a trap containing 2N NaOH aq. solution to quench any excess phosgene). The reaction mixture was then diluted with water (30 mL) and extracted with DCM (3×20 mL). The combined organic phases were washed with brine (30 mL), dried over Na₂SO₄, filtered, and concentrated in vacuo. The crude product was purified by column chromatography (0-35% EtOAc+20% EtOH in cyclohexane) to afford the title compound (1.34 g). [M+H]⁺ m/z 489.4

### Intermediate 127

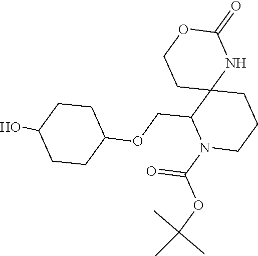 [SMILES: CC(C)(C)OC(=O)N1CCCC2(CCOC(=O)N2)C1COC1CCC(O)CC1]

### tert-butyl-rel-(6R,7R)-7-{[(4-hydroxycyclohexyl)oxy]methyl}-2-oxo-3-oxa-1,8-diazaspiro[5.5]undecane-8-carboxylate

Intermediate 127 was prepared following a similar procedure as described for Intermediate 8 to afford the title compound (859 mg) as a colorless oil. [M+H]⁺ m/z 399.5

### Intermediate 128

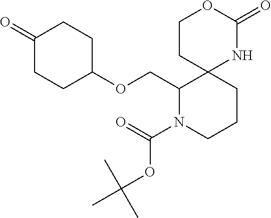 [SMILES: CC(C)(C)OC(=O)N1CCCC2(CCOC(=O)N2)C1COC1CCC(=O)CC1]

### tert-butyl-rel-(6R,7R)-2-oxo-7-{[(4-oxocyclohexyl)oxy]methyl}-3-oxa-1,8-diazaspiro[5.5]undecane-8-carboxylate

Intermediate 128 was prepared following a similar procedure as described for Intermediate 46 to afford the title compound (1.22 g). [M+H]⁺ m/z 397.5

### Intermediate 129

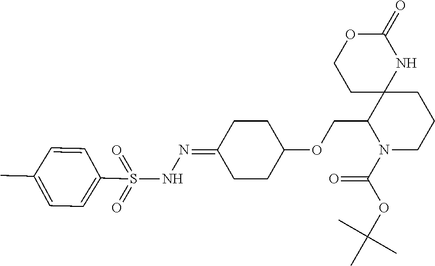 [SMILES: Cc1ccc(S(=O)(=O)NN=C2CCC(OCC3N(C(=O)OC(C)(C)C)CCCC34CCOC(=O)N4)CC2)cc1]

### tert-butyl-rel-(6R,7R)-7-[({4-[(4-methylbenzenesulfonamido)imino]cyclohexyl}oxy)methyl]-2-oxo-3-oxa-1,8-diazaspiro[5.5]undecane-8-carboxylate

Intermediate 129 was prepared following a similar procedure as described for Intermediate 47 to afford the title compound (1.1 g). [M+H]⁺ m/z 565.5

### Intermediate 130

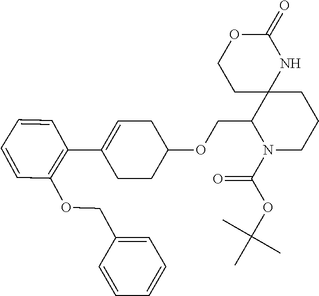 [SMILES: CC(C)(C)OC(=O)N1CCCC2(CCOC(=O)N2)C1COC1CC=C(c2ccccc2OCc2ccccc2)CC1]

### tert-butyl-rel-(6R,7R)-7-[({4-[2-(benzyloxy)phenyl]cyclohex-3-en-1-yl}oxy)methyl]-2-oxo-3-oxa-1,8-diazaspiro[5.5]undecane-8-carboxylate

Intermediate 130 was prepared following a similar procedure as described for Intermediate 48 to afford the title compound (50 mg). [M+H]⁺ m/z 563.3

### Intermediate 131

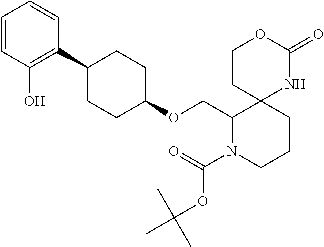 [SMILES: CC(C)(C)OC(=O)N1CCCC2(CCOC(=O)N2)C1CO[C@H]1CC[C@@H](c2ccccc2O)CC1]

### tert-butyl-rel-(6R, 7R)-2-oxo-7-({[(1s,4s)-4-(2-hydroxyphenyl)cyclohexyl]oxy}methyl)-3-oxa-1,8-diazaspiro[5.5]undecane-8-carboxylate

Intermediate 131 was prepared following a similar procedure as described for Intermediate 49 to afford the title compound (45 mg). [M+H]⁺ m/z 475.3

### Intermediate 132

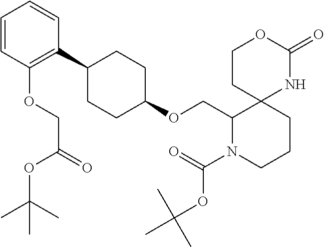 [SMILES: CC(C)(C)OC(=O)COc1ccccc1[C@H]1CC[C@@H](OCC2N(C(=O)OC(C)(C)C)CCCC23CCOC(=O)N3)CC1]

### tert-butyl-rel-(6R,7R)-2-oxo-7-({[(1s,4s)-4-{2-[2-(tert-butoxy)-2-oxoethoxy]phenyl}cyclohexyl]oxy}methyl)-3-oxa-1,8-diazaspiro[5.5]undecane-8-carboxylate

Intermediate 132 was prepared following a similar procedure as described for Intermediate 67 to afford the title compound (38 mg). [M+H]⁺ m/z 589.3

### Intermediate 133

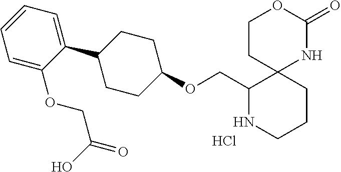

### 2-{2-[(1s,4s)-4-{[rel-(6R,7R)-2-oxo-3-oxa-1,8-diazaspiro[5.5]undecan-7-yl]methoxy}cyclohexyl]phenoxy}acetic acid hydrochloride

Intermediate 133 was prepared following a similar procedure as described for Intermediate 57 to afford the title compound (28 mg). [M+H]⁺ m/z 433.3

### Example 57

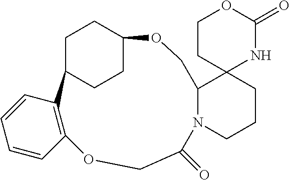 [SMILES: O=C1NC2(CCCN3C(=O)COc4ccccc4[C@H]4CC[C@H](CC4)OCC32)CCO1]

### Rel-(1′s,4R,16′R,19′s)-8′,18′-dioxa-11′-azaspiro[1,3-oxazinane-4,15′-tetracyclo[17.2.2.0^{2,7}0.0^{11,16}]tricosane]-2′(7′),3′,5′-triene-2,10′-dione

Example 57 was prepared using Intermediate 133 following a similar procedure as described for Example 1 to afford the title compound (2.2 mg).

LCMS (Method C): [M+H]⁺ m/z 415.2, RT 0.92 min

¹H NMR (500 MHZ, CDCl₃) δ 7.14-7.24 (m, 1H), 7.05-7.13 (m, 1H), 6.91 (br t, J=7.4 Hz, 1H), 6.75 (br d, J=8.0 Hz, 1H), 6.35 (br s, 1H), 5.09 (br d, J=10.4 Hz, 2H), 4.62 (br t, J=12.0 Hz, 1H), 4.34-4.41 (m, 1H), 4.31 (br d, J=10.4 Hz, 1H), 3.93 (br t, J=9.5 Hz, 1H), 3.75 (br s, 1H), 3.70 (br d, J=12.8 Hz, 1H), 3.58 (br d, J=4.8 Hz, 1H), 3.44-3.54 (m, 1H), 2.52-2.72 (m, 1H), 1.21-2.77 (m, 14H).

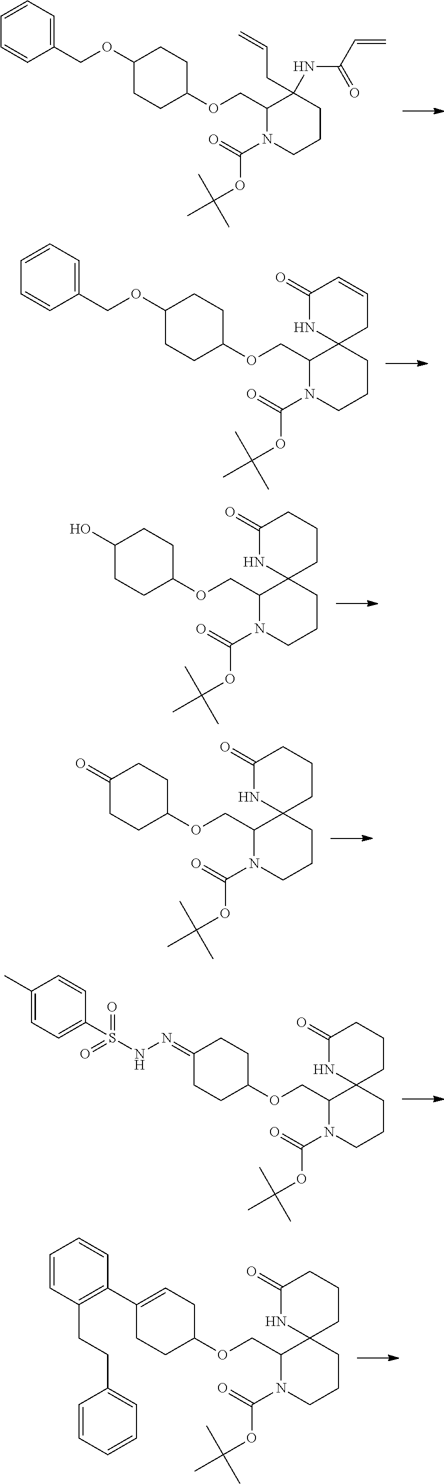

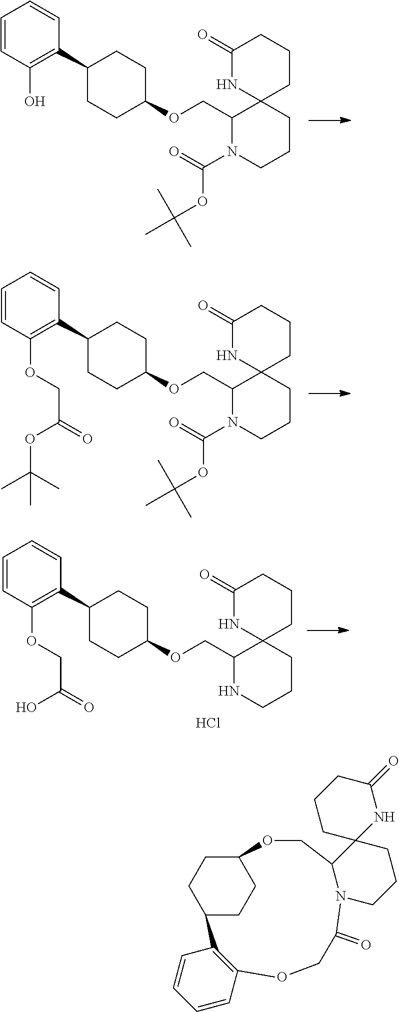

### Intermediate 134

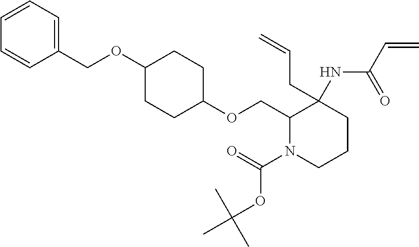 [SMILES: C=CCC1(NC(=O)C=C)CCCN(C(=O)OC(C)(C)C)C1COC1CCC(OCc2ccccc2)CC1]

### tert-butyl-rel-(2R,3R)-2-({[4-(benzyloxy)cyclohexyl]oxy}methyl)-3-(prop-2-en-1-yl)-3-(prop-2-enamido) piperidine-1-carboxylate

To a stirred solution of Intermediate 121 (2.65 g) in MeTHF (53 mL), triethylamine (5.3 mL, 38.03 mmol) and a 1 M solution of 2-propenoyl chloride (4.62 mL, 4.62 mmol) in MeTHF were added at 0° C. The solution was stirred at 0° C. for 1 h. Further triethylamine (3.0 mL, 21.52 mmol) and 2-propenoyl chloride (1.0 mL, 1 mmol, 1 M sol. in MeTHF) were added and the mixture was stirred at 0° C. for 1 h. The reaction was quenched by addition of sat. aq. NaHCO₃ (100 mL) and extracted with EtOAc (2×100 mL). The combined organic phases were dried (Na₂SO₄) and concentrated in vacuo. The crude product was purified by column chromatography (0-30% EtOAc in cyclohexane) to afford the title compound (1.5 g) as a colorless thick oil. [M+H]⁺ m/z 513.4

### Intermediate 135

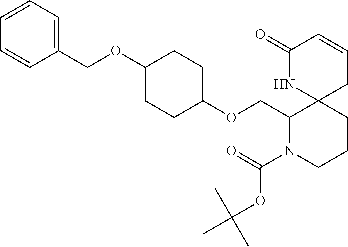 [SMILES: CC(C)(C)OC(=O)N1CCCC2(CC=CC(=O)N2)C1COC1CCC(OCc2ccccc2)CC1]

### tert-butyl-rel-(6R,7R)-7-({[4-(benzyloxy)cyclohexyl]oxy}methyl)-2-oxo-1,8-diazaspiro[5.5]undec-3-ene-8-carboxylate

A solution of Intermediate 134 (1.62 g) in toluene (632 mL) was degassed by bubbling N₂ (g) for 15 minutes. The degassed solution was heated at 65° C. and Zhan Catalyst-1B (100 mg, 0.140 mmol) was added. The pale-yellow solution was stirred at the same temperature for 3 h with a slow N₂ flow. The reaction mixture was concentrated in vacuo and the crude product was purified by column chromatography (0-50% EtOAc in cyclohexane) to afford the title compound (1.23 g) as a pale-yellow oil. [M+H]⁺ m/z 485.4

### Intermediate 136

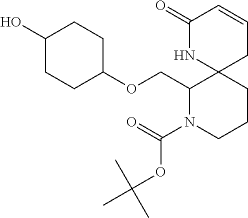 [SMILES: CC(C)(C)OC(=O)N1CCCC2(CC=CC(=O)N2)C1COC1CCC(O)CC1]

### tert-butyl-rel-(6S,7R)-7-{[(4-hydroxycyclohexyl)oxy]methyl}-2-oxo-1,8-diazaspiro[5.5]undecane-8-carboxylate

Intermediate 136 was prepared following a similar procedure as described for Intermediate 8 to afford the title compound (1.02 g) as an off-white solid. [M+H]⁺ m/z 397.4

### Intermediate 137

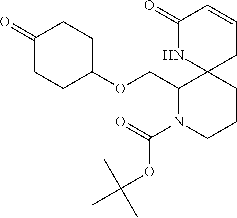 [SMILES: CC(C)(C)OC(=O)N1CCCC2(CC=CC(=O)N2)C1COC1CCC(=O)CC1]

### tert-butyl-rel-(6S,7R)-2-oxo-7-{[(4-oxocyclohexyl)oxy]methyl}-1,8-diazaspiro[5.5]-undecane-8-carboxylate

Intermediate 137 was prepared following a similar procedure as described for Intermediate 46 to afford the title compound (1.6 g) as a colorless oil. [M+H]⁺ m/z 395.4

### Intermediate 138

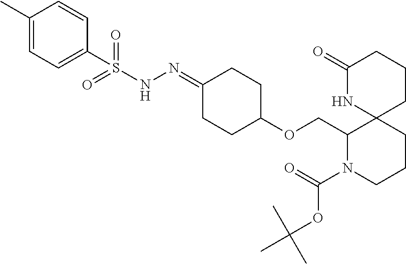 [SMILES: Cc1ccc(S(=O)(=O)NN=C2CCC(OCC3N(C(=O)OC(C)(C)C)CCCC34CCCC(=O)N4)CC2)cc1]

### tert-butyl-rel-(6S,7R)-7-[({4-[(4-methylbenzenesulfonamido)imino]cyclohexyl}oxy)methyl]-2-oxo-1,8-diazaspiro[5.5]undecane-8-carboxylate

Intermediate 138 was prepared following a similar procedure as described for Intermediate 47 to afford the title compound (1.02 g) as a white solid. [M+H]⁺ m/z 563.4

### Intermediate 139

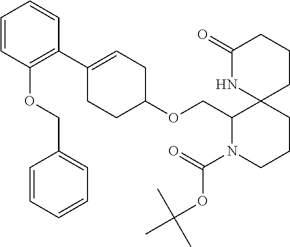 [SMILES: CC(C)(C)OC(=O)N1CCCC2(CCCC(=O)N2)C1COC1CC=C(c2ccccc2OCc2ccccc2)CC1]

### tert-butyl-rel-(6S, 7R)-7-[({4-[2-(benzyloxy)phenyl]cyclohex-3-en-1-yl}oxy)methyl]-2-oxo-1,8-diazaspiro[5.5]undecane-8-carboxylate

Intermediate 139 was prepared following a similar procedure as described for Intermediate 48 to afford the title compound (345 mg) as a yellow oil. [M+H]⁺ m/z 561.5

### Intermediate 140

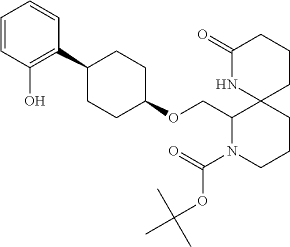 [SMILES: CC(C)(C)OC(=O)N1CCCC2(CCCC(=O)N2)C1CO[C@H]1CC[C@@H](c2ccccc2O)CC1]

### tert-butyl-rel-(6S, 7R)-2-oxo-7-({[(1s,4s)-4-(2-hydroxyphenyl)cyclohexyl]oxy}methyl)-1,8-diazaspiro[5.5]undecane-8-carboxylate

Intermediate 140 was prepared following a similar procedure as described for Intermediate 8 to afford the title compound (140 mg) as a yellow oil. [M+H]⁺ m/z 473.4

### Intermediate 141

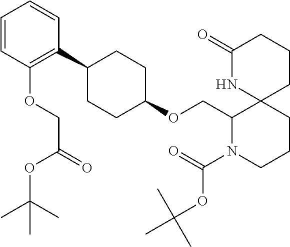 [SMILES: CC(C)(C)OC(=O)COc1ccccc1[C@H]1CC[C@@H](OCC2N(C(=O)OC(C)(C)C)CCCC23CCCC(=O)N3)CC1]

### tert-butyl-rel-(6S, 7R)-2-oxo-7-({[(1s,4s)-4-{2-[2-(tert-butoxy)-2-oxoethoxy]phenyl}cyclohexyl]oxy}methyl)-1,8-diazaspiro[5.5]undecane-8-carboxylate

Intermediate 141 was prepared following a similar procedure as described for Intermediate 67 to afford the title compound (120 mg) as a yellow oil. [M+H]⁺ m/z 587.5

### Intermediate 142

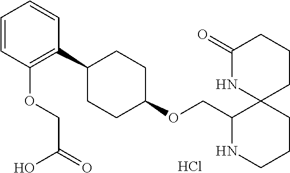 [SMILES: O=C(O)COc1ccccc1[C@H]1CC[C@@H](OCC2NCCCC23CCCC(=O)N3)CC1 ; Cl]

### 2-{2-[(1s,4s)-4-{[rel-(6S,7R)-2-oxo-1,8-diazaspiro[5.5]undecan-7-yl]methoxy}cyclohexyl]phenoxy}acetic acid hydrochloride

Intermediate 142 was prepared following a similar procedure as described for Intermediate 68 to afford the title compound (120 mg) as a yellow oil. [M−HCl+H]⁺ m/z 431.4

### Example 58

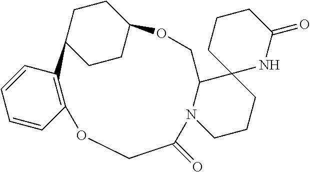 [SMILES: O=C1CCCC2(CCCN3C(=O)COc4ccccc4[C@H]4CC[C@H](CC4)OCC32)N1]

### Rel-(1′s,2S,16′R,19′s)-8′,18′-dioxa-11′-azaspiro[piperidine-2,15′-tetracyclo-[17.2.2.0^{2,7}0.0^{11,16}]tricosane]-2′(7′),3′,5′-triene-6,10′-dione

Example 58 was prepared using Intermediate 142 following a similar procedure as described for Example 1 to afford the title compound (35 mg) as a white solid.

LCMS (Method C): [M+H]⁺ m/z413.4, RT 0.95 min

¹H NMR (500 MHZ, CDCl₃) δ 7.17 (td, J=7.7, 1.7 Hz, 1H), 7.10 (dd, J=7.4, 1.5 Hz, 1H), 6.90 (td, J=7.4, 1.0 Hz, 1H), 6.75 (d, J=8.0 Hz, 1H), 6.25 (br s, 1H), 5.13 (dd, J=10.9, 3.6 Hz, 1H), 5.10 (d, J=10.6 Hz, 1H), 4.31 (d, J=10.6 Hz, 1H), 3.90 (dd, J=10.8, 8.6 Hz, 1H), 3.71 (br s, 1H), 3.74-3.66 (m, 1H), 3.53 (dd, J=8.4, 4.0 Hz, 1H), 3.52-3.43 (m, 1H), 2.71-2.61 (m, 1H), 2.62-2.53 (m, 1H), 2.53-2.44 (m, 1H), 2.37-2.28 (m, 2H), 2.29-2.21 (m, 1H), 2.20-2.13 (m, 1H), 2.12-2.05 (m, 1H), 1.94-1.85 (m, 2H), 1.83-1.69 (m, 4H), 1.54-1.47 (m, 1H), 1.46-1.31 (m, 4H).

### Example 58a and 58b

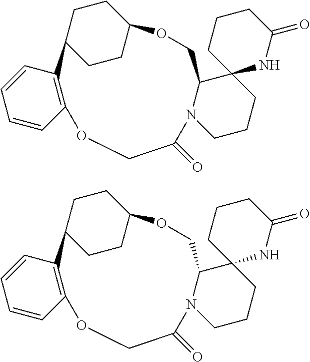

### Example 58a: (1′s,2S,16′R,19′s)-8′,18′-dioxa-11′-azaspiro[piperidine-2,15′-tetracyclo-[17.2.2.0^{2,7}0.0^{11,16}]tricosane]-2′(7′),3′,5′-triene-6,10′-dione

### Example 58b: (1′s,2R,16′S,19′s)-8′,18′-dioxa-11′-azaspiro[piperidine-2,15′-tetracyclo-[17.2.2.0^{2,7}0.0^{11,16}]tricosane]-2′(7′),3′,5′-triene-6,10′-dione

Example 58 (33 mg, 0.080 mmol) was subjected to chiral preparative purification using Waters 600 and Chiralpak AD-H, 25×2.0 cm, 5 μm column eluting with 65:35 Heptane: (Ethanol/Methanol 1/1+0.1 isopropylamine) to afford the title compounds (Peak 1, 9.52 mg, 100% ee; and Peak 2, 9.96 mg, 100% ee).

### Example 58a: Peak 1 (Stereochemistry Tentatively Assigned)

LCMS (Method C): [M+H]⁺ m/z 413.3, RT 0.95 min

Chiral analysis (Chiralpak AD-H, 25×0.46 cm, 5 μm, 65:35 n-Hexane: (Ethanol/Methanol 1/1+0.1 isopropylamine): RT 6.4 minutes

¹H NMR (500 MHZ, CDCl₃) δ 7.17 (td, J=7.7, 1.7 Hz, 1H), 7.10 (dd, J=7.4, 1.5 Hz, 1H), 6.90 (td, J=7.4, 1.0 Hz, 1H), 6.75 (d, J=8.0 Hz, 1H), 6.25 (br s, 1H), 5.13 (dd, J=10.9, 3.6 Hz, 1H), 5.10 (d, J=10.6 Hz, 1H), 4.31 (d, J=10.6 Hz, 1H), 3.90 (dd, J=10.8, 8.6 Hz, 1H), 3.71 (br s, 1H), 3.74-3.66 (m, 1H), 3.53 (dd, J=8.4, 4.0 Hz, 1H), 3.52-3.43 (m, 1H), 2.71-2.61 (m, 1H), 2.62-2.53 (m, 1H), 2.53-2.44 (m, 1H), 2.37-2.28 (m, 2H), 2.29-2.21 (m, 1H), 2.20-2.13 (m, 1H), 2.12-2.05 (m, 1H), 1.94-1.85 (m, 2H), 1.83-1.69 (m, 4H), 1.54-1.47 (m, 1H), 1.46-1.31 (m, 4H).

### Example 58b: Peak 2 (Stereochemistry Tentatively Assigned)

LCMS (Method C): [M+H]⁺ m/z 413.3, RT 0.94 min

Chiral analysis (Chiralpak AD-H, 25×0.46 cm, 5 μm, 65:35 n-Hexane: (Ethanol/Methanol 1/1+0.1 isopropylamine): RT 9.7 minutes

¹H NMR (500 MHZ, CDCl₃) δ 7.17 (td, J=7.7, 1.7 Hz, 1H), 7.10 (dd, J=7.4, 1.5 Hz, 1H), 6.90 (td, J=7.4, 1.0 Hz, 1H), 6.75 (d, J=8.0 Hz, 1H), 6.25 (br s, 1H), 5.13 (dd, J=10.9, 3.6 Hz, 1H), 5.10 (d, J=10.6 Hz, 1H), 4.31 (d, J=10.6 Hz, 1H), 3.90 (dd, J=10.8, 8.6 Hz, 1H), 3.71 (br s, 1H), 3.74-3.66 (m, 1H), 3.53 (dd, J=8.4, 4.0 Hz, 1H), 3.52-3.43 (m, 1H), 2.71-2.61 (m, 1H), 2.62-2.53 (m, 1H), 2.53-2.44 (m, 1H), 2.37-2.28 (m, 2H), 2.29-2.21 (m, 1H), 2.20-2.13 (m, 1H), 2.12-2.05 (m, 1H), 1.94-1.85 (m, 2H), 1.83-1.69 (m, 4H), 1.54-1.47 (m, 1H), 1.46-1.31 (m, 4H).

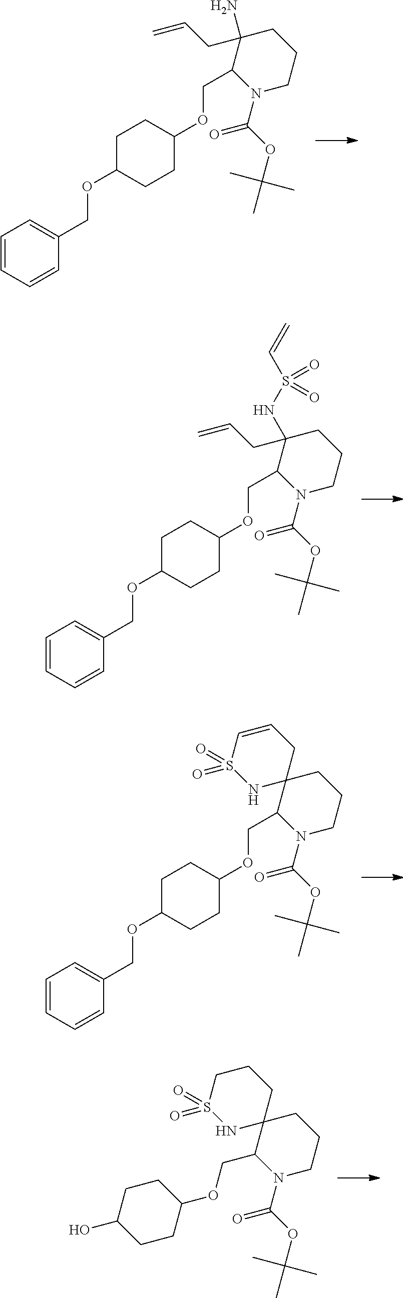 [SMILES: C=CCC1(N)CCCN(C(=O)OC(C)(C)C)C1COC1CCC(OCc2ccccc2)CC1 ; C=CCC1(NS(=O)(=O)C=C)CCCN(C(=O)OC(C)(C)C)C1COC1CCC(OCc2ccccc2)CC1 ; CC(C)(C)OC(=O)N1CCCC2(CC=CS(=O)(=O)N2)C1COC1CCC(OCc2ccccc2)CC1 ; CC(C)(C)OC(=O)N1CCCC2(CCCS(=O)(=O)N2)C1COC1CCC(O)CC1]

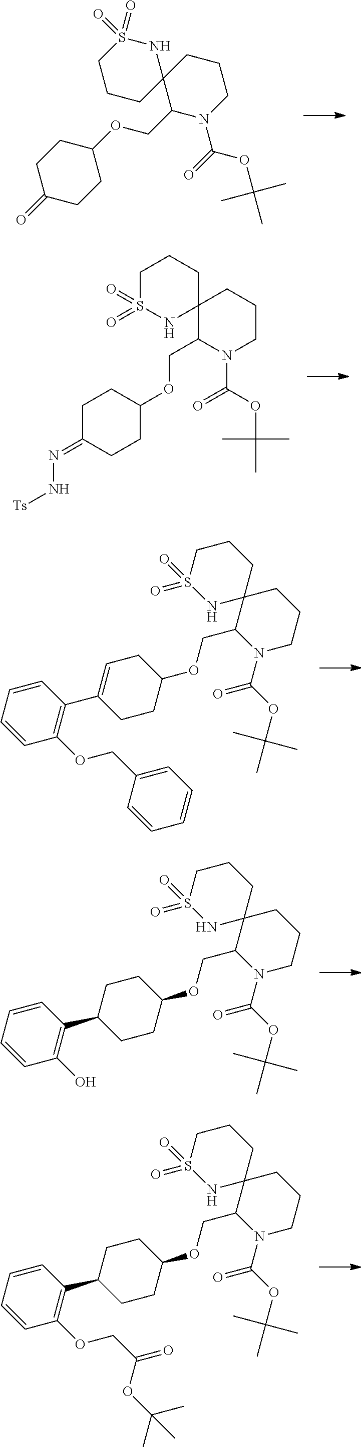

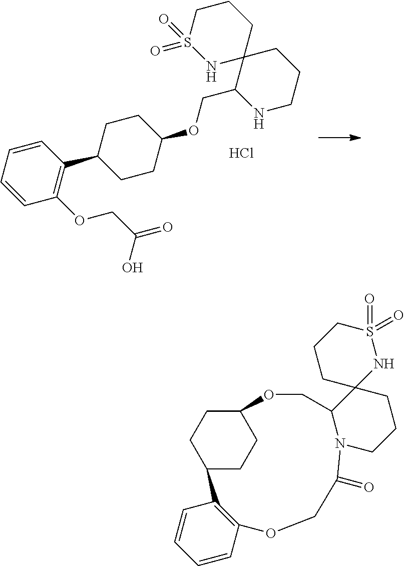 [SMILES: O=C(O)COc1ccccc1[C@H]1CC[C@@H](OCC2NCCCC23CCCS(=O)(=O)N3)CC1 ; Cl ; O=C1COc2ccccc2[C@H]2CC[C@H](CC2)OCC2N1CCCC21CCCS(=O)(=O)N1]

### Intermediate 143

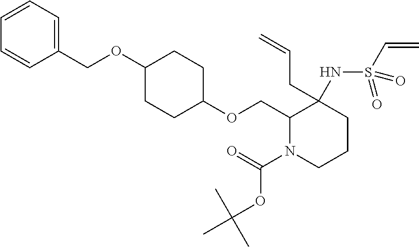 [SMILES: C=CCC1(NS(=O)(=O)C=C)CCCN(C(=O)OC(C)(C)C)C1COC1CCC(OCc2ccccc2)CC1]

### tert-butyl-rel-(2R,3R)-2-({[4-(benzyloxy)cyclohexyl]oxy}methyl)-3-ethenesulfonamido-3-(prop-2-en-1-yl)piperidine-1-carboxylate

To a stirred solution of Intermediate 121 (3.65 g) and triethylamine (3.33 mL, 23.88 mmol) in dry DCM (79 mL) was added 2-chloroethanesulfonyl chloride (1.67 mL, 15.92 mmol) at 0° C. and the mixture was stirred for 30 min. The reaction mixture was cooled to room temperature, quenched with a solution of saturated aqueous NH₄Cl, diluted with water and extracted with DCM. The combined organic extracts were washed with brine and concentrated in vacuo. The crude product was purified by column chromatography (0-100% EtOAc in cyclohexane) to afford the title compound (3.28 g) as a colorless gum. [M+H]⁺ m/z 549.4

### Intermediate 144

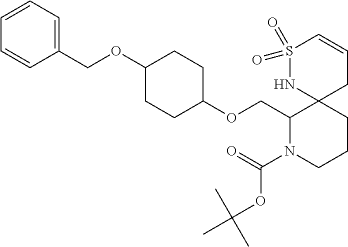 [SMILES: CC(C)(C)OC(=O)N1CCCC2(CC=CS(=O)(=O)N2)C1COC1CCC(OCc2ccccc2)CC1]

### tert-butyl-rel-(6R,7R)-7-({[4-(benzyloxy)cyclohexyl]oxy}methyl)-2,2-dioxo-2λ⁶-thia-1,8-diazaspiro[5.5]undec-3-ene-8-carboxylate

Intermediate 144 was prepared following a similar procedure as described for Intermediate 135 to afford the title compound (2.81 g) as a pale-yellow oil. [M+H]⁺ m/z 521.4

### Intermediate 145

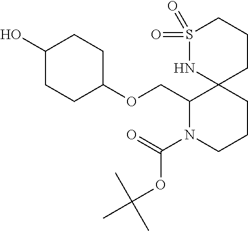 [SMILES: CC(C)(C)OC(=O)N1CCCC2(CCCS(=O)(=O)N2)C1COC1CCC(O)CC1]

### tert-butyl-rel-(6R, 7R)-7-{[(4-hydroxycyclohexyl)oxy]methyl}-2,2-dioxo-216-thia-1,8-diazaspiro[5.5]undecane-8-carboxylate

Intermediate 145 was prepared following a similar procedure as described for Intermediate 8 to afford the title compound (2.1 g) as a colorless oil. [M+H]⁺ m/z 433.3

### Intermediate 146

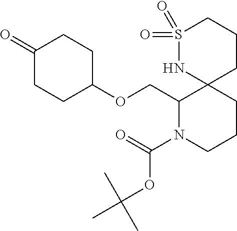 [SMILES: CC(C)(C)OC(=O)N1CCCC2(CCCS(=O)(=O)N2)C1COC1CCC(=O)CC1]

### tert-butyl-rel-(6R,7R)-2,2-dioxo-7-{[(4-oxocyclohexyl)oxy]methyl}-216-thia-1,8-diazaspiro[5.5]undecane-8-carboxylate

Intermediate 146 was prepared following a similar procedure as described for Intermediate 46 to afford the title compound (2.8 g) as a colorless oil. [M+H]⁺ m/z 431.4

### Intermediate 147

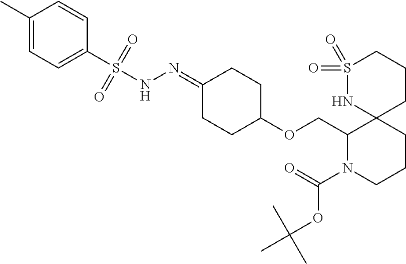 [SMILES: Cc1ccc(S(=O)(=O)NN=C2CCC(OCC3N(C(=O)OC(C)(C)C)CCCC34CCCS(=O)(=O)N4)CC2)cc1]

### tert-butyl-rel-(6R, 7R)-7-[({4-[(4-methylbenzenesulfonamido)imino]cyclohexyl}oxy)methyl]-2,2-dioxo-2λ⁶-thia-1,8-diazaspiro[5.5]undecane-8-carboxylate

Intermediate 147 was prepared following a similar procedure as described for Intermediate 47 to afford the title compound (1.87 g) as a white solid. [M+H]⁺ m/z 599.4

### Intermediate 148

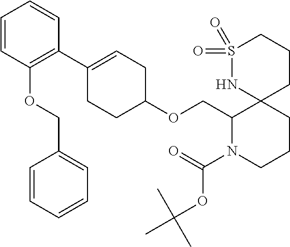 [SMILES: CC(C)(C)OC(=O)N1CCCC2(CCCS(=O)(=O)N2)C1COC1CC=C(c2ccccc2OCc2ccccc2)CC1]

### tert-butyl-rel-(6R, 7R)-7-[({4-[2-(benzyloxy)phenyl]cyclohex-3-en-1-yl}oxy)methyl]-2,2-dioxo-216-thia-1,8-diazaspiro[5.5]undecane-8-carboxylate

Intermediate 148 was prepared following a similar procedure as described for Intermediate 47 to afford the title compound (1.16 g) as a yellow oil. [M+H]⁺ m/z 597.5

### Intermediate 149

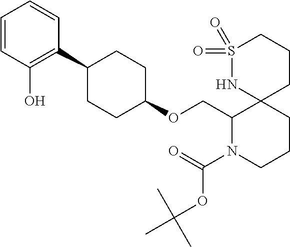 [SMILES: CC(C)(C)OC(=O)N1CCCC2(CCCS(=O)(=O)N2)C1CO[C@H]1CC[C@@H](c2ccccc2O)CC1]

### tert-butyl-rel-(6R,7R)-2,2-dioxo-7-({[(1s,4s)-4-(2-hydroxyphenyl)cyclohexyl]oxy}methyl)-216-thia-1,8-diazaspiro[5.5]undecane-8-carboxylate

Intermediate 149 was prepared following a similar procedure as described for Intermediate 8 to afford the title compound (880 mg) as a white solid. [M+H]⁺ m/z 509.4

### Intermediate 150

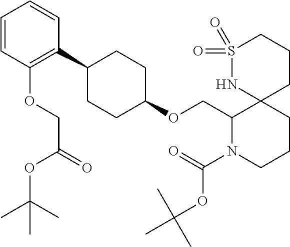 [SMILES: CC(C)(C)OC(=O)COc1ccccc1[C@H]1CC[C@@H](OCC2N(C(=O)OC(C)(C)C)CCCC23CCCS(=O)(=O)N3)CC1]

### tert-butyl-rel-(6R,7R)-2,2-dioxo-7-({[(1s,4s)-4-{2-[2-(tert-butoxy)-2-oxoethoxy]-phenyl}cyclohexyl]oxy}methyl)-26-thia-1,8-diazaspiro[5.5]undecane-8-carboxylate

Intermediate 150 was prepared following a similar procedure as described for Intermediate 67 to afford the title compound (312 mg) as a colorless oil. [M+H]⁺ m/z 623.4

### Intermediate 151

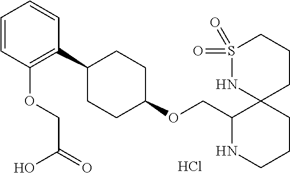 [SMILES: O=C(O)COc1ccccc1[C@H]1CC[C@@H](OCC2NCCCC23CCCS(=O)(=O)N3)CC1 ; Cl]

### 2-{2-[(1s,4s)-4-{[rel-(6R, 7R)-2,2-dioxo-2λ⁶-thia-1,8-diazaspiro[5.5]undecan-7-yl]methoxy}cyclohexyl]phenoxy}acetic acid

Intermediate 151 was prepared following a similar procedure as described for Intermediate 68 to afford the title compound (265 mg) as a white solid. [M+H]⁺ m/z 467.3

### Example 59

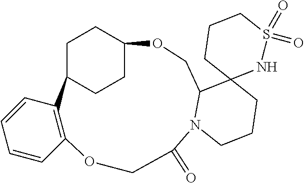 [SMILES: O=C1COc2ccccc2[C@H]2CC[C@H](CC2)OCC2N1CCCC21CCCS(=O)(=O)N1]

### Rel-(1s, 15R,16R,19s)-8,18-dioxa-11-azaspiro[tetracyclo[17.2.2.0^{2,7}0.0^{11,16}]tricosane-15,3′-[1λ⁶,2]thiazinane]-2 (7),3,5-triene-1′,1′,10-trione

Example 59 was prepared using Intermediate 151 following a similar procedure as described for Example 1 to afford the title compound (50 mg) as a white solid.

LCMS (Method C): [M+H]⁺ m/z449.3, RT 1.06 min

¹H NMR (500 MHZ, CDCl₃) δ 7.17 (td, J=7.7, 1.6 Hz, 1H), 7.10 (dd, J=7.3, 1.5 Hz, 1H), 6.90 (td, J=7.4, 0.8 Hz, 1H), 6.71-6.78 (m, 1H), 5.38 (br dd, J=9.6, 4.1 Hz, 1H), 5.08 (d, J=10.7 Hz, 1H), 4.31 (d, J=10.7 Hz, 1H), 4.15 (br s, 1H), 3.88-3.95 (m, 1H), 3.80-3.88 (m, 1H), 3.75 (br s, 1H), 3.66-3.73 (m, 1H), 3.51-3.62 (m, 1H), 3.18-3.25 (m, 1H), 2.96 (ddd, J=13.1, 11.3, 3.6 Hz, 1H), 2.61-2.73 (m, 2H), 2.51-2.62 (m, 1H), 2.18-2.33 (m, 3H), 2.05 (br d, J=14.0 Hz, 1H), 1.86-1.98 (m, 2H), 1.21-1.83 (m, 8H).

### Examples 59a and 59b

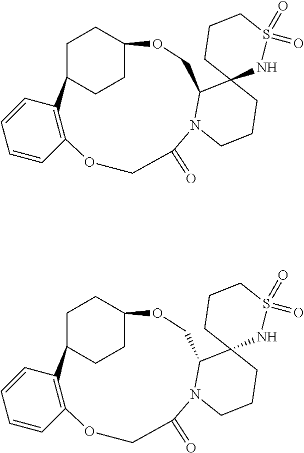 [SMILES: O=C1COc2ccccc2[C@H]2CC[C@H](CC2)OC[C@@H]2N1CCC[C@@]21CCCS(=O)(=O)N1 ; O=C1COc2ccccc2[C@H]2CC[C@H](CC2)OC[C@H]2N1CCC[C@]21CCCS(=O)(=O)N1]

### Example 59a: (1s, 15R,16R,19s)-8,18-dioxa-11-azaspiro[tetracyclo[17.2.2.0^{2,7}0.0^{11,16}]-tricosane-15,3′-[1λ⁶,2]thiazinane]-2 (7),3,5-triene-1′,1′,10-trione

### Example 59b: (1s, 15S,16S,19s)-8, 18-dioxa-11-azaspiro[tetracyclo[17.2.2.0^{2,7}0.0^{11,16}]-tricosane-15,3′-[1λ⁶,2]thiazinane]-2 (7),3,5-triene-1′,1′,10-trione

Example 59 (48 mg, 0.107 mmol) was subjected to chiral preparative purification using Waters 600 and Chiralcel OD-H, 25×2.0 cm, 5 μm column eluting with 70:30 Heptane: Ethanol +0.1 isopropylamine to afford the title compounds (Peak 1, 20.8 mg, 97.4% ee; and Peak 2, 19.3 mg, 100% ee).

### Example 59a: Peak 1 (Stereochemistry Tentatively Assigned)

LCMS (Method C): [M+H]⁺ m/z 449.2, RT 1.06 min

Chiral analysis (Chiralcel OD-H, 25×0.46 cm, 5 μm, 70:30 n-Hexane: Ethanol+0.1 isopropylamine: RT 11.9 minutes

¹H NMR (500 MHZ, CDCl₃) δ 7.17 (td, J=7.7, 1.6 Hz, 1H), 7.10 (dd, J=7.3, 1.5 Hz, 1H), 6.90 (td, J=7.4, 0.8 Hz, 1H), 6.71-6.78 (m, 1H), 5.38 (br dd, J=9.6, 4.1 Hz, 1H), 5.08 (d, J=10.7 Hz, 1H), 4.31 (d, J=10.7 Hz, 1H), 4.15 (br s, 1H), 3.88-3.95 (m, 1H), 3.80-3.88 (m, 1H), 3.75 (br s, 1H), 3.66-3.73 (m, 1H), 3.51-3.62 (m, 1H), 3.18-3.25 (m, 1H), 2.96 (ddd, J=13.1, 11.3, 3.6 Hz, 1H), 2.61-2.73 (m, 2H), 2.51-2.62 (m, 1H), 2.18-2.33 (m, 3H), 2.05 (br d, J=14.0 Hz, 1H), 1.86-1.98 (m, 2H), 1.21-1.83 (m, 8H).

### Example 59b: Peak 2 (Stereochemistry Tentatively Assigned)

LCMS (Method C): [M+H]⁺ m/z 449.2, RT 1.06 min

Chiral analysis (Chiralcel OD-H, 25×0.46 cm, 5 μm, 70:30 n-Hexane: Ethanol+0.1 isopropylamine: RT 15.5 minutes

¹H NMR (500 MHZ, CDCl₃) δ 7.17 (td, J=7.7, 1.6 Hz, 1H), 7.10 (dd, J=7.3, 1.5 Hz, 1H), 6.90 (td, J=7.4, 0.8 Hz, 1H), 6.71-6.78 (m, 1H), 5.38 (br dd, J=9.6, 4.1 Hz, 1H), 5.08 (d, J=10.7 Hz, 1H), 4.31 (d, J=10.7 Hz, 1H), 4.15 (br s, 1H), 3.88-3.95 (m, 1H), 3.80-3.88 (m, 1H), 3.75 (br s, 1H), 3.66-3.73 (m, 1H), 3.51-3.62 (m, 1H), 3.18-3.25 (m, 1H), 2.96 (ddd, J=13.1, 11.3, 3.6 Hz, 1H), 2.61-2.73 (m, 2H), 2.51-2.62 (m, 1H), 2.18-2.33 (m, 3H), 2.05 (br d, J=14.0 Hz, 1H), 1.86-1.98 (m, 2H), 1.21-1.83 (m, 8H).

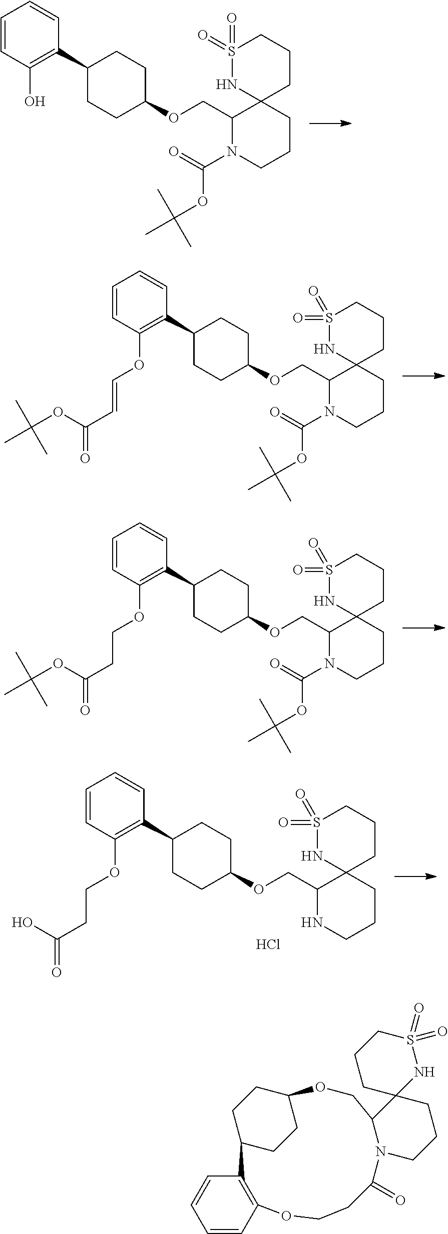

### Intermediate 152

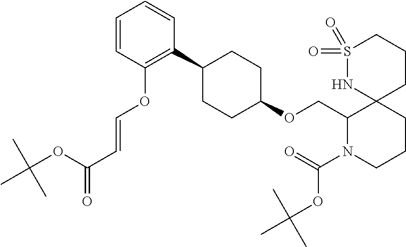 [SMILES: CC(C)(C)OC(=O)/C=C/Oc1ccccc1[C@H]1CC[C@@H](OCC2N(C(=O)OC(C)(C)C)CCCC23CCCS(=O)(=O)N3)CC1]

### tert-butyl-rel-(6R, 7R)-2,2-dioxo-7-({[(1s,4s)-4-(2-{[(1E)-3-(tert-butoxy)-3-oxoprop-1-en-1-yl]oxy}phenyl)cyclohexyl]oxy}methyl)-2λ⁶-thia-1,8-diazaspiro[5.5]undecane-8-carboxylate

Intermediate 154 was prepared from Intermediate 149 following a similar procedure as described for Intermediate 30 to afford the title compound (150 mg) as a pale-yellow oil. [M+H]⁺ m/z 635.4

### Intermediate 153

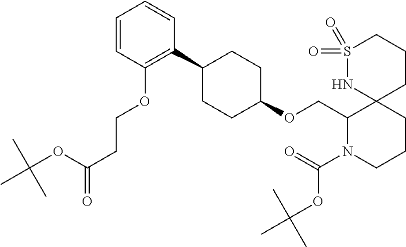 [SMILES: CC(C)(C)OC(=O)CCOc1ccccc1[C@H]1CC[C@@H](OCC2N(C(=O)OC(C)(C)C)CCCC23CCCS(=O)(=O)N3)CC1]

### tert-butyl-rel-(6R,7R)-2,2-dioxo-7-({[(1s,4s)-4-{2-[3-(tert-butoxy)-3-oxopropoxy]phenyl}cyclohexyl]oxy}methyl)-2λ⁶-thia-1,8-diazaspiro[5.5]undecane-8-carboxylate

Intermediate 153 was prepared following a similar procedure as described for Intermediate 8 to afford the title compound (125 mg) as a colorless oil. [M+H]⁺ m/z 637.4

### Intermediate 154

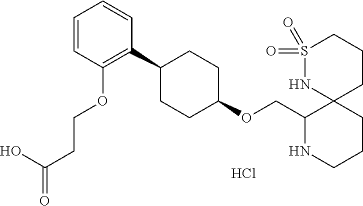 [SMILES: O=C(O)CCOc1ccccc1[C@H]1CC[C@@H](OCC2NCCCC23CCCS(=O)(=O)N3)CC1 ; Cl]

3-{2-[(1s,4s)-4-{[rel-(6R,7R)-2,2-dioxo-2λ⁶-thia-1,8-diazaspiro[5.5]undecan-7-yl]methoxy}cyclohexyl]phenoxy}propanoic acid hydrochloride

Intermediate 154 was prepared following a similar procedure as described for Intermediate 68 to afford the title compound (125 mg) as a pale-yellow solid. [M+H]⁺ m/z 481.3

### Example 60

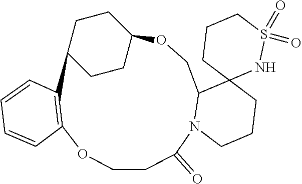 [SMILES: O=C1CCOc2ccccc2[C@H]2CC[C@H](CC2)OCC2N1CCCC21CCCS(=O)(=O)N1]

### Rel-(1s, 16R,17R,20s)-8,19-dioxa-12-azaspiro[tetracyclo[18.2.2.0^{2,7}0.0^{12,17}]tetracosane-16,3′-[1λ⁶,2]thiazinane]-2 (7),3,5-triene-1′,1′,11-trione

Example 60 was prepared using Intermediate 156 following a similar procedure as described for Example 1 to afford the title compound (44 mg) as a white solid.

LCMS (Method C): [M+H]⁺ m/z 463.3, RT 1.07 min

¹H NMR (500 MHZ, CDCl₃) δ 7.12 (td, J=7.8, 1.4 Hz, 1H), 7.07-7.00 (m, 1H), 6.88-6.79 (m, 1H), 6.84-6.76 (m, 1H), 5.60-4.84 (m, 1H), 4.80-4.23 (m, 1H), 4.07-4.01 (m, 1H), 4.46-3.97 (m, 1H), 4.16-3.94 (m, 1H), 4.82-3.78 (m, 1H), 3.94-3.73 (m, 1H), 3.69-3.59 (m, 1H), 3.27-3.18 (m, 1H), 3.00-2.84 (m, 1H), 3.41-2.71 (m, 1H), 3.61-2.39 (m, 1H), 2.49-2.39 (m, 1H), 3.18-2.15 (m, 1H), 2.71-1.19 (m, 16H).

### Example 61

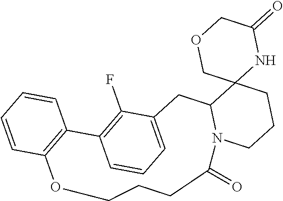 [SMILES: O=C1COCC2(CCCN3C(=O)CCCOc4ccccc4-c4cccc(c4F)CC32)N1]

### Rel-(3S,18′S)-24′-fluoro-8′-oxa-13′-azaspiro[morpholine-3,17′-tetracyclo[18.3.1.0^{2,7}0.0^{13,18}]tetracosane]-1′(24′),2′(7′), 3′,5′,20′,22′-hexaene-5,12′-dione

Example 61 was prepared using commercially available starting material tert-butyl 3-oxopiperidine-1-carboxylate following a similar procedure as described for example 27 to afford the title compound (9.9 mg) as a white solid.

LCMS (Method B): [M+H]⁺ m/z 439.3, RT 2.63 minutes

¹H NMR (400 MHZ, CDCl₃) δ 7.42-6.56 (m, 8H), 5.26-4.36 (m, 1H), 4.34-3.52 (m, 4H), 3.47-2.48 (m, 4H), 2.48-1.78 (m, 3H), 1.79-1.07 (m, 7H).

### Example 62

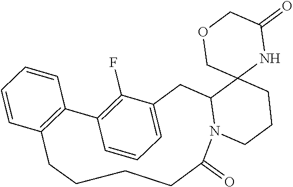 [SMILES: O=C1COCC2(CCCN3C(=O)CCCCc4ccccc4-c4cccc(c4F)CC32)N1]

### Rel-(3S, 18′S)-24′-fluoro-13′-azaspiro[morpholine-3,17′-tetracyclo[18.3.1.0^{2,7}0.0^{13,18}]tetracosane]-1′(24′),2′(7′),3′,5′,20′,22′-hexaene-5,12′-dione

Example 62 was prepared using commercially available starting material tert-butyl 3-oxopiperidine-1-carboxylate following a similar procedure as described for example 29 to afford the title compound (14.9 mg) as a white solid.

LCMS (Method B): [M+H]⁺ m/z 437.0, RT 3.00 minutes

¹H NMR (400 MHZ, CDCl₃) δ 7.42-6.56 (m, 8H), 5.26-4.36 (m, 1H), 4.34-3.52 (m, 4H), 3.47-2.48 (m, 4H), 2.48-1.78 (m, 3H), 1.79-1.07 (m, 7H).

### Example 63

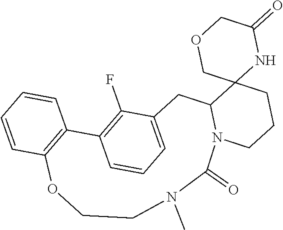 [SMILES: CN1CCOc2ccccc2-c2cccc(c2F)CC2N(CCCC23COCC(=O)N3)C1=O]

### Rel-(3S,18′S)-24′-fluoro-11′-methyl-8′-oxa-11′,13′-diazaspiro[morpholine-3,17′-tetracyclo[18.3.1.0^{2,7}0.0^{13,18}]tetracosane]-1′(24′),2′(7′),3′,5′,20′,22′-hexaene-5,12′-dione

Example 63 was prepared using commercially available starting material tert-butyl 3-oxopiperidine-1-carboxylate following a similar procedure as described for example 30 to afford the title compound (1 mg) as a white solid.

LCMS (Method B): [M+H]⁺ m/z 454.3, RT 2.73 minutes

¹H NMR (500 MHZ, CDCl₃) δ 7.43-7.31 (m, 1H), 7.27-6.83 (m, 6H), 6.13-5.91 (m, 1H), 4.77-4.50 (m, 1H), 4.39-4.26 (m, 1H), 4.24-4.06 (m, 2H), 3.96-3.86 (m, 1H), 3.53-3.44 (m, 1H), 3.42-3.30 (m, 1H), 3.26-3.16 (m, 1H), 3.04-2.89 (m, 1H), 2.87-2.71 (m, 1H), 2.70-2.59 (m, 1H), 2.58-2.43 (m, 1H), 1.96-1.78 (m, 2H), 1.51-1.42 (m, 1H), 1.34-1.22 (m, 3H), 0.93-0.80 (m, 2H).

### Example 64

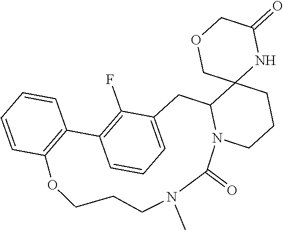 [SMILES: CN1CCCOc2ccccc2-c2cccc(c2F)CC2N(CCCC23COCC(=O)N3)C1=O]

### Rel-(3S,19′S)-25′-fluoro-12′-methyl-8′-oxa-12′,14′-diazaspiro[morpholine-3,18′-tetracyclo[19.3.1.0^{2,7}0.0^{14,19}]pentacosane]-1′(25′),2′(7′),3′,5′,21′,23′-hexaene-5,13′-dione

Example 64 was prepared using commercially available starting material tert-butyl 3-oxopiperidine-1-carboxylate following a similar procedure as described for example 31 to afford the title compound (37.2 mg) as a white solid.

LCMS (Method A): [M+H]⁺ m/z 486.3, RT 3.01 minutes

¹H NMR (500 MHZ, CDCl₃) δ 7.36 (td, J=7.9, 1.6 Hz, 1H), 7.26-7.20 (m, 2H), 7.14-7.06 (m, 2H), 7.06-7.00 (m, 1H), 6.96 (dd, J=8.0, 4.8 Hz, 1H), 6.31-6.13 (m, 1H), 4.72 (dd, J=12.8, 3.1 Hz, 1H), 4.39-4.32 (m, 2H), 4.16 (d, J=16.9 Hz, 1H), 4.06-3.95 (m, 1H), 3.66-3.57 (m, 1H), 3.54-3.42 (m, 3H), 3.42-3.32 (m, 1H), 2.98-2.88 (m, 2H), 2.67 (d, J=13.3 Hz, 1H), 2.56 (s, 3H), 1.97-1.39 (m, 6H).

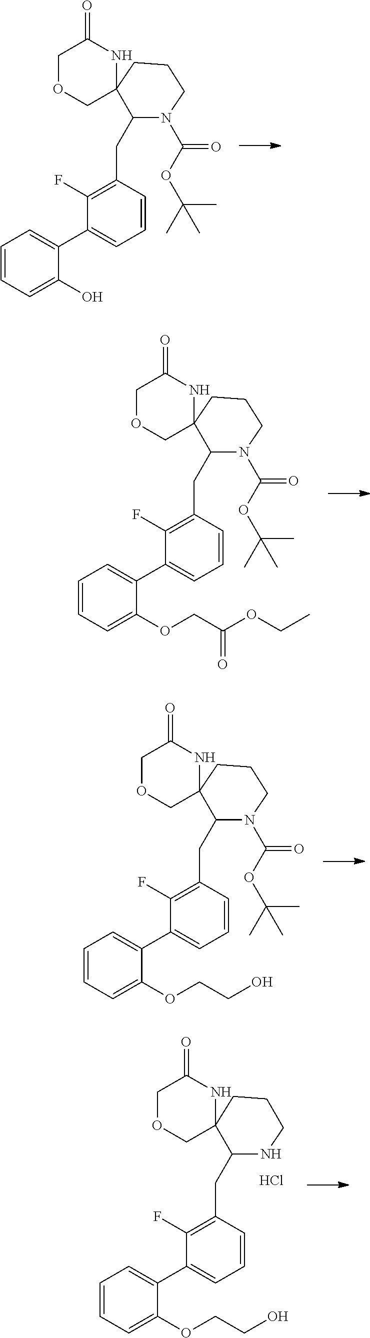

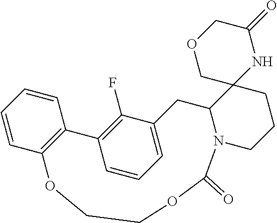 [SMILES: O=C1COCC2(CCCN3C(=O)OCCOc4ccccc4-c4cccc(c4F)CC32)N1]

### Intermediate 155

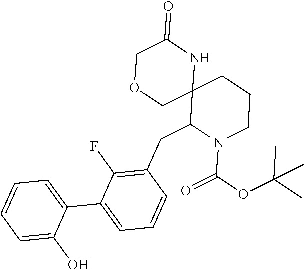 [SMILES: CC(C)(C)OC(=O)N1CCCC2(COCC(=O)N2)C1Cc1cccc(-c2ccccc2O)c1F]

### tert-butyl-rel-(6S,7S)-7-({2-fluoro-2′-hydroxy-[1,1′-biphenyl]-3-yl}methyl)-2-oxo-4-oxa-1,8-diazaspiro[5.5]undecane-8-carboxylate

Intermediate 155 was prepared using commercially available starting material tert-butyl 3-oxopiperidine-1-carboxylate following a similar procedure as described for Intermediate 78 to afford the title compound (108 mg) as a white solid. [M+H]⁺ m/z 471.3

### Intermediate 156

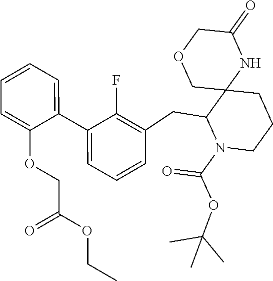 [SMILES: CCOC(=O)COc1ccccc1-c1cccc(CC2N(C(=O)OC(C)(C)C)CCCC23COCC(=O)N3)c1F]

### tert-butyl-rel-(6S,7S)-7-{[2′-(2-ethoxy-2-oxoethoxy)-2-fluoro-[1,1′-biphenyl]-3-yl]methyl}-2-oxo-4-oxa-1,8-diazaspiro[5.5]undecane-8-carboxylate

To a solution of Intermediate 155 (300 mg) and ethyl bromoacetate (0.095 mL, 0.861 mmol) in acetone (3.0259 mL) was added dipotassium carbonate (239 mg, 1.73 mmol) and the solution was heated to 50° C. overnight. The solids were filtered off and the filtrate was concentrated in vacuo then the residue was suspended in water (5 mL) and extracted with DCM (3×5 mL). The combined organic phases were concentrated in vacuo to afford the title compound (332 mg) as a pale brown solid. [M+H]⁺ m/z 557.3

### Intermediate 157

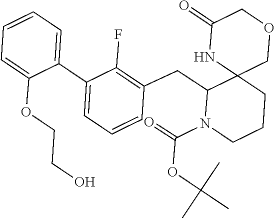 [SMILES: CC(C)(C)OC(=O)N1CCCC2(COCC(=O)N2)C1Cc1cccc(-c2ccccc2OCCO)c1F]

### tert-butyl-rel-(6S,7S)-7-{[2-fluoro-2′-(2-hydroxyethoxy)-[1,1′-biphenyl]-3-yl]methyl}-2-oxo-4-oxa-1,8-diazaspiro[5.5]undecane-8-carboxylate

2 M Lithium borohydride in THF (0.27 mL, 0.531 mmol) was added dropwise to a stirred solution of Intermediate 156 (332 mg) in anhydrous THF (5 mL) at 0° C. and the mixture was stirred for 20 h at room temperature. Further 2 M lithium borohydride (0.27 mL, 0.531 mmol) was added and the reaction mixture was stirred at room temperature for 2 hours. The reaction mixture was quenched with water (10 mL) and extracted with EtOAc (3×10 mL). The combined organic extracts were dried (MgSO₄) and concentrated in vacuo to afford the title compound (284 mg) as a colorless solid. [M+H]⁺ m/z 515.2

### Intermediate 158

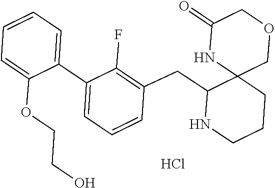 [SMILES: O=C1COCC2(CCCNC2Cc2cccc(-c3ccccc3OCCO)c2F)N1 ; Cl]

### Rel-(6S,7S)-7-{[2-fluoro-2′-(2-hydroxyethoxy)-[1,1′-biphenyl]-3-yl]methyl}-4-oxa-1,8-diazaspiro[5.5]undecan-2-one hydrochloride

4 M Hydrogen chloride in dioxane (6.0 mL, 24.0 mmol) was added to Intermediate 157 (284 mg) at room temperature and the reaction mixture was stirred for 1 hour. The reaction mixture was concentrated in vacuo. The residue was resuspended with water (10 ml) and DCM (10 mL). The organic phase was discarded and the aqueous phase was washed with further DCM (10 mL). The pH of the aqueous phase was adjusted to ˜pH10 with 2M NaOH. The aqueous phase was extracted with DCM (3×10 mL). These combined organic extracts were passed through a hydrophobic frit and concentrated in vacuo to afford the title compound (233 mg) as a colorless solid. [M+H]⁺ m/z 415.3

### Example 65

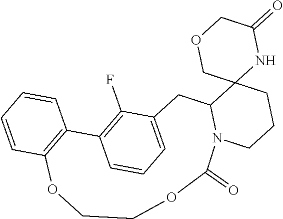 [SMILES: O=C1COCC2(CCCN3C(=O)OCCOc4ccccc4-c4cccc(c4F)CC32)N1]

### Rel-(3S, 18′S)-24′-fluoro-8′,11′-dioxa-13′-azaspiro[morpholine-3,17′-tetracyclo[18.3.1.0^{2,7}0.0^{13,18}]tetracosane]-1′(24′),2′(7′),3′,5′,20′,22′-hexaene-5,12′-dione

To a solution of Intermediate 158 (233 mg) and DIPEA (0.83 mL, 4.78 mmol) in anhydrous acetonitrile (27.049 mL) was added bis(trichloromethyl) carbonate (47 mg, 0.158 mmol) in anhydrous acetonitrile (8.791 mL) over 2 hours. The mixture was stirred at room temperature for 30 mins. The reaction mixture was then heated to 80° C. for 18 hours. The reaction mixture was cooled to room temperature before being concentrated in vacuo to afford the crude material. The crude material was purified by Acidic Preparative HPLC. Acidic Early Elute Method: Waters Sunfire C18 column (30 mm×100 mm, 5 μm; temperature: room temperature). Injection volume of 1500 μL at a flow rate of 40 mL/min. 10% B (A=0.1% formic acid in water; B=0.1% formic acid in acetonitrile) for 1.90 min then a gradient of 10-95% B over 14.1 min and held for 1.9 min. A second gradient of 95-10% B was then applied over 0.3 min and held for a further 0.9 min. UV spectra were recorded at 215 nm using a Gilson detector. The appropriate fractions were collected combined and concentrated to afford the desired product at ˜70% purity. The crude material was re-purified by reverse phase column chromatography (10-100% MeCN (0.1% Formic acid) in Water (0.1% Formic acid) to afford the title compound (8.9 mg) as a colorless solid.

LCMS (Method A): [M+H]⁺ m/z 441.2, RT 2.71 and 2.90 minutes

¹H NMR (500 MHZ, CDCl₃) δ 7.35 (q, J=8.5 Hz, 1H), 7.24 (s, 1H), 7.15 (q, J=7.2 Hz, 3H), 7.04 (q, J=7.2 Hz, 1H), 6.84 (d, J=8.2 Hz, 1H), 6.63 (s, 1H), 5.25-5.16 (m, 1H), 4.77 (d, J=11.9 Hz, 1H), 4.37-4.27 (m, 1H), 4.16-4.08 (m, 3H), 4.06-3.98 (m, 2H), 3.80 (d, J=12.3 Hz, 1H), 3.40 (d, J=11.8 Hz, 1H), 3.36-3.22 (m, 2H), 2.65 (d, J=13.1 Hz, 1H), 1.83-1.62 (m, 3H), 1.60-1.48 (m, 1H).

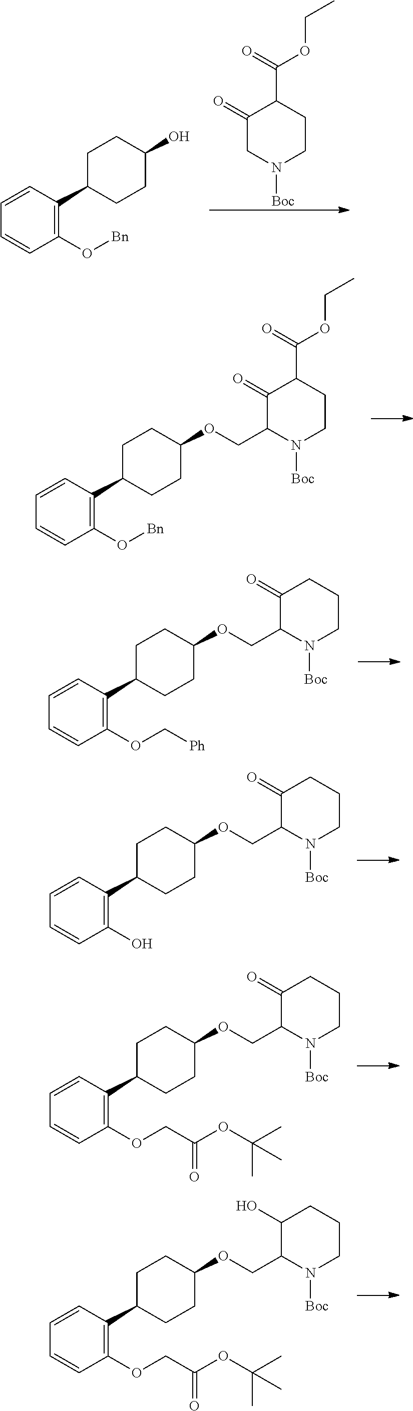

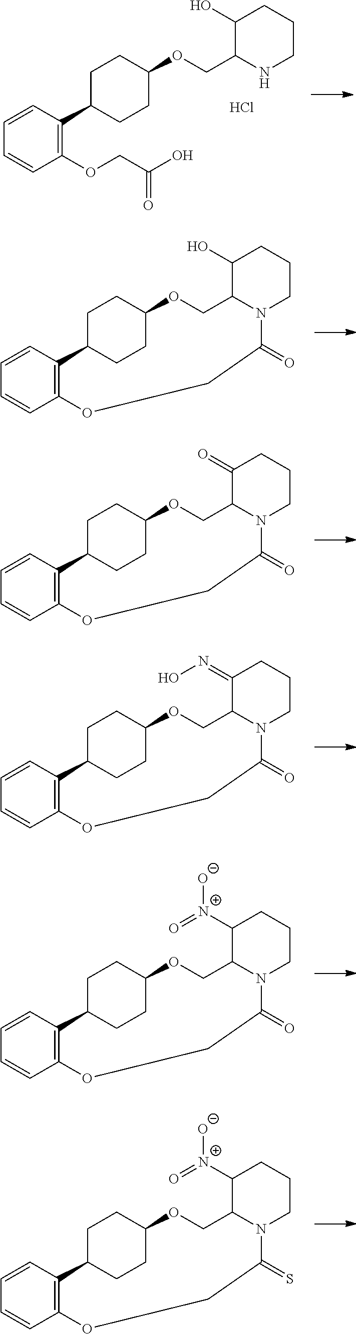

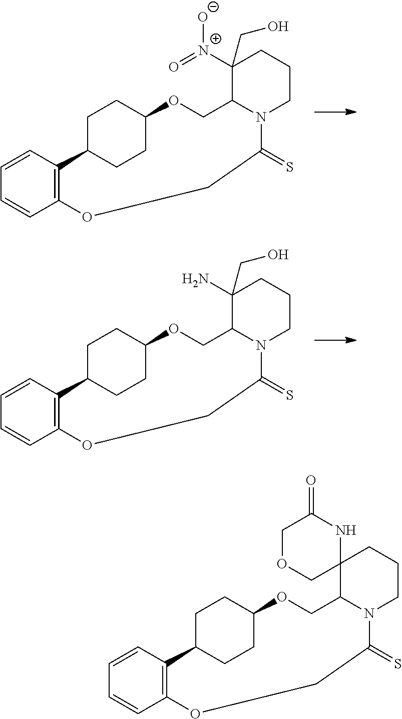 [SMILES: O=[N+]([O-])C1(CO)CCCN2C(=S)COc3ccccc3[C@H]3CC[C@H](CC3)OCC21 ; NC1(CO)CCCN2C(=S)COc3ccccc3[C@H]3CC[C@H](CC3)OCC21 ; O=C1COCC2(CCCN3C(=S)COc4ccccc4[C@H]4CC[C@H](CC4)OCC32)N1]

### Intermediate 159

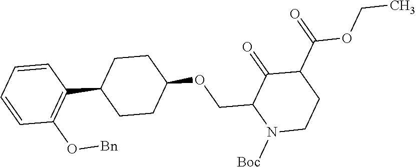 [SMILES: CCOC(=O)C1CCN(C(=O)OC(C)(C)C)C(CO[C@H]2CC[C@@H](c3ccccc3OCc3ccccc3)CC2)C1=O]

### 1-tert-butyl 4-ethyl 3-oxo-2-({[(1s,4s)-4-[2-(benzyloxy)phenyl]-cyclohexyl]oxy}methyl)-piperidine-1,4-dicarboxylate

In a flask, 2.4 M butyllithium in THF (79 mL, 0.191 mol) was added to a stirred solution of N-(propan-2-yl)propan-2-amine (26 mL, 0.188 mol) in anhydrous THF (90 mL) at −78° C. The reaction was held at this temperature for 40 min. In a second flask the freshly made LDA was added, via an addition funnel over 0.5 h, to a stirred solution of 1-tert-butyl 4-ethyl 3-oxopiperidine-1,4-dicarboxylate (24.00 g, 86.7 mmol) and 1,3-dimethylhexahydropyrimidin-2-one (42 mL, 0.345 mol) in anhydrous THF (60 mL) at −78° C., the reaction temperature did not rise above −70° C. The solution was held at this temperature for 20 mins. The oil containing 1-benzyloxy-2-[4-(chloromethoxy)cyclohexyl]benzene (28.65 g, 86.6 mmol) in anhydrous THF (90 mL) was added to the reaction mixture over 20 mins, the reaction temperature did not rise above-70° C. The reaction mixture was stirred at −78° C. for 1 h, warmed to room temperature and stirred for 2 hours. The reaction was quenched with NH₄Cl. The crude mixture was diluted with water (100 mL) and extracted with EtOAc (3×300 mL). The combined extracts were dried (MgSO₄), filtered, and concentrated in vacuo. The crude material was purified by silica gel column chromatography (0-50% EtOAc in heptane) to afford the title compound (15 g) as a pale-yellow oil. [M+Na]⁺ m/z 588.3

### Intermediate 160

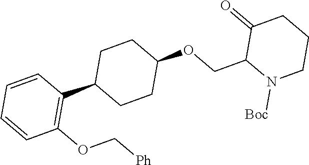 [SMILES: CC(C)(C)OC(=O)N1CCCC(=O)C1CO[C@H]1CC[C@@H](c2ccccc2OCc2ccccc2)CC1]

### tert-butyl 3-oxo-2-({[(1s,4s)-4-[2-(benzyloxy)phenyl]cyclohexyl]oxy}methyl)piperidine-1-carboxylate

To Intermediate 159 (15.00 g) in DMSO (95 mL) was added sodium chloride (2.26 g, 38.6 mmol) and water (9.5 mL) and the reaction mixture was heated to 125° C. for 2.5 h. The reaction mixture was cooled to room temperature, quenched water (200 mL) and extracted with EtOAc (2×200 mL). The combined organic extracts were washed with water (2×200 mL) and brine (200 mL), dried over magnesium sulfate, filtered, and concentrated to dryness to afford crude material. The crude material was purified by column chromatography (0-50% EtOAc in heptane) to afford the title compound (7.03 g) as an orange oil. [M+Na]⁺ m/z 516.3

### Intermediate 161

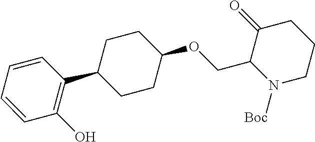 [SMILES: CC(C)(C)OC(=O)N1CCCC(=O)C1CO[C@H]1CC[C@@H](c2ccccc2O)CC1]

### tert-butyl 3-oxo-2-({[(1s,4s)-4-(2-hydroxyphenyl)cyclohexyl]oxy}methyl)piperidine-1-carboxylate

Intermediate 160 (17.4 g) were dissolved in ethanol (300 mL) and the atmosphere was evacuated and backfilled with nitrogen 3 times. Palladium on carbon (10%, 3.57 g, 3.36 mmol) was added and the atmosphere was evacuated and backfilled with hydrogen 3 times. The reaction was stirred for 18 hours and then filtered through Celite, washing with ethyl acetate, and the filtrate was concentrated in vacuo to afford the title compound (13.9 g) as a yellow oil. [M+Na]⁺ m/z 426.3

### Intermediate 162

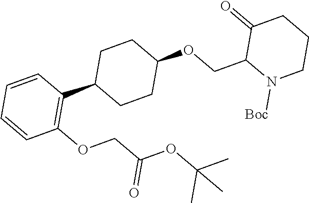 [SMILES: CC(C)(C)OC(=O)COc1ccccc1[C@H]1CC[C@@H](OCC2C(=O)CCCN2C(=O)OC(C)(C)C)CC1]

### tert-butyl 3-oxo-2-({[(1s,4s)-4-(2-hydroxyphenyl)cyclohexyl]oxy}methyl)piperidine-1-carboxylate

To a solution of Intermediate 161 (11.80 g) and tert-butyl bromoacetate (5.0 mL, 33.6 mmol) in acetone (152 mL) was added dipotassium carbonate (12.18 g, 88.1 mmol) and the solution was stirred at room temperature overnight. The solids were filtered off and the filtrate was concentrated in vacuo to afford the title compound (15.50 g) as a colorless oil. [M+Na]⁺ m/z 540.3

### Intermediate 163

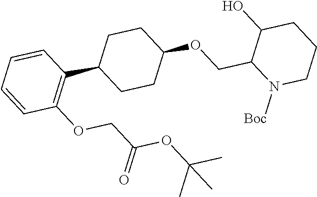 [SMILES: CC(C)(C)OC(=O)COc1ccccc1[C@H]1CC[C@@H](OCC2C(O)CCCN2C(=O)OC(C)(C)C)CC1]

### tert-butyl-3-hydroxy-2-({[(1s,4s)-4-{2-[2-(tert-butoxy)-2-ethoxy]-phenyl}cyclohexyl]oxy}methyl)piperidine-1-carboxylate

Sodium borohydride (1.07 g, 28.3 mmol) was added portionwise to a stirred solution of Intermediate 162 (15.50 g) in anhydrous DCM (118 mL) and methanol (118 mL) at 0° C. and the mixture was stirred for 2 h. The reaction mixture was concentrated in vacuo. The residue was resuspended in water (300 mL) and extracted with DCM (3×200 mL). The combined organic extracts were dried over MgSO₄, filtered, and concentrated in vacuo to afford the title compound (14.7 g) as a white solid. [M+H]⁺ m/z 542.3

### Intermediate 164

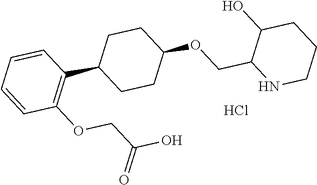 [SMILES: O=C(O)COc1ccccc1[C@H]1CC[C@@H](OCC2NCCCC2O)CC1 ; Cl]

### 2-{2-[(1s,4s)-4-[(3-hydroxypiperidin-2-yl) methoxy]cyclohexyl]phenoxy}acetic acid hydrochloride

4 M Hydrogen chloride in dioxane (69 mL, 0.277 mol) was added to Intermediate 163 (14.70 g) at 0° C. The reaction mixture was allowed to warm to room temperature and stirred for 4 hours. The solvent was removed in vacuo to afford the title compound (11.5 g) as a white solid. [M+H]⁺ m/z 362.3

### Intermediate 165

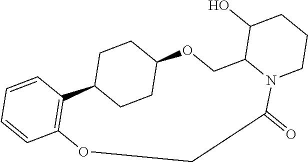 [SMILES: O=C1COc2ccccc2[C@H]2CC[C@H](CC2)OCC2C(O)CCCN12]

### (1s, 19s)-15-hydroxy-8, 18-dioxa-11-azatetracyclo[17.2.2.0^{2,7}0.0^{11,16}]tricosa-2 (7),3,5-trien-10-one

To a stirred solution of HATU (15.00 g, 39.4 mmol) and DIPEA (23 mL, 0.132 mol) in acetonitrile (1240 mL) was added Intermediate 164 (10.50 g) in anhydrous DMF (124 mL) over 2h using a syringe pump. The resulting solution was stirred for one hour. The reaction mixture was concentrated in vacuo to afford the crude material. The crude material was diluted in water (200 mL) and extracted with EtOAc (3×200 mL). The combined organic extracts were washed with brine (200 mL), dried (MgSO₄), filtered and concentrated to give the crude product. The crude material was purified by column chromatography (0-40% 3:1 EtOAc/EtOH in heptane) to afford the title compound (5.9 g) as a white solid. [M+H]⁺ m/z 346.28.

### Intermediate 166

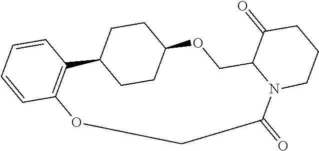 [SMILES: O=C1CCCN2C(=O)COc3ccccc3[C@H]3CC[C@H](CC3)OCC12]

### (1s, 19s)-8, 18-dioxa-11-azatetracyclo[17.2.2.0^{2,7}0.0^{11,16}]tricosa-2 (7),3,5-triene-10,15-dione

To a solution of Intermediate 165 (5.90 g) in anhydrous DCM (130.64 mL) at 0° C. was added Dess-Martin periodinane (8.76 g, 20.6 mmol). The mixture was stirred at room temperature for 1 hour. The mixture was diluted with DCM (200 mL), then washed with sat. aqueous NaHCO₃ (100 ml), 1M aqueous Na₂S₂O₃ (100 mL), and 1M aqueous Na₂CO₃ (100 mL). The organic phase was dried by passing through a hydrophobic frit and evaporated to dryness to give the crude product. The crude product was purified by column chromatography (0-100% 3:1 EtOAc/EtOH in heptane) to afford the title compound (4.1 g) as a pale-yellow solid. [M+H]⁺ m/z 344.2

### Intermediate 167

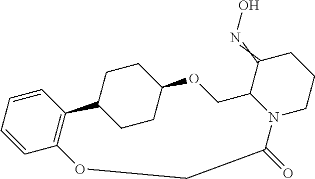

### (1s, 19s)-15-(hydroxyimino)-8,18-dioxa-11-azatetracyclo[17.2.2.0^{2,7}0.0^{11,16}]tricosa-2 (7),3,5-trien-10-one

A solution of triethylamine (0.72 mL, 5.19 mmol), hydroxylamine hydrochloride (1:1) (360 mg, 5.18 mmol) and Intermediate 166 (600 mg) in ethanol (6 mL) was heated to 90° C. for 1 h. The reaction mixture was diluted with water (5 mL) and extracted with EtOAc (3×5 mL). The combined organic extracts were dried over magnesium sulfate and concentrated in vacuo to afford the title compound (630 mg) as a white solid. [M+H]⁺ m/z 359.3

### Intermediate 168

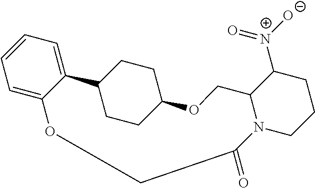

### (1s, 19s)-15-nitro-8, 18-dioxa-11-azatetracyclo[17.2.2.0^{2,7}0.0^{11,16}]tricosa-2,4,6-trien-10-one

A solution of trifluoroacetic anhydride (0.61 mL, 4.41 mmol) in acetonitrile (3 mL) was added to a stirred solution of hydrogen peroxide-urea (1:1) (562 mg, 5.97 mmol) in acetonitrile (3 mL) at 0° C. and the mixture was stirred at 0° C. for 2 h. The resulting solution was added dropwise to a mixture of Intermediate 167 (630 mg) and sodium hydrogen carbonate (724 mg, 8.62 mmol) in acetonitrile (15 mL) at 80° C. for 1 h. The reaction mixture was cooled to room temperature, quenched with sat. aqueous Na₂SO₃ (10 mL) and stirred for 10 min then extracted with EtOAc (3×20 mL). The combined organic extracts were dried over MgSO₄, filtered and concentrated in vacuo to afford the title compound (650 mg) as a yellow solid. [M+H]⁺ m/z 375.2.

### Intermediate 169

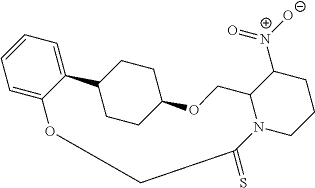

### (1s, 19s)-15-nitro-8,18-dioxa-11-azatetracyclo[17.2.2.0^{2,7}0.0^{11,16}]tricosa-2,4,6-triene-10-thione

2,4-bis(4-Methoxyphenyl)-1,3,2,4-dithiadiphosphetane 2,4-disulfide (162 mg, 0.401 mmol) was added to Intermediate 168 (100 mg) and the reaction mixture was stirred for 2 hours at 100° C. The reaction mixture was filtered and the filtrate was concentrated in vacuo. The crude product was purified by column chromatography (0-100% EtOAc in heptane) to afford the title compound (150 mg) as a yellow oil. [M+H]⁺ m/z 391.2

### Intermediate 170

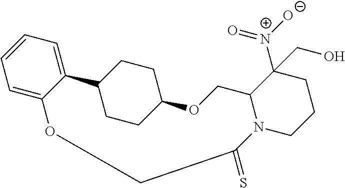

### Rel-(1s, 15S,16R,19s)-15-(hydroxymethyl)-15-nitro-8, 18-dioxa-11-azatetracyclo[17.2.2.0^{2,7}0.0^{11,16}]tricosa-2,4,6-triene-10-thione

Formaldehyde (37% in water, 0.19 mL, 2.53 mmol) was added to Intermediate 169 (150 mg) and triethylamine (0.047 mL, 0.339 mmol) in THF (1.6216 mL) at room temperature. The solution was heated to 70° C. for 18 h. After cooling the reaction mixture was diluted with water (20 mL) and extracted with EtOAc (3×20 mL). The combined organic extracts were washed with brine (20 mL), dried (MgSO₄), filtered and concentrated in vacuo. The crude product was purified by silica gel column chromatography (0-60% EtOAc in heptane) to afford the title compound (65 mg) as a yellow solid. [M+H]⁺ m/z 421.2

### Intermediate 171

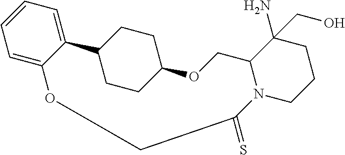

### Rel-(1s, 15S,16R,19s)-15-amino-15-(hydroxymethyl)-8,18-dioxa-11-azatetracyclo[17.2.2.0^{2,7}0.0^{11,16}]tricosa-2,4,6-triene-10-thione

A suspension of Intermediate 170 (64 mg) and zinc (100 mg, 1.53 mmol) in acetic acid (0.45 mL) and ethanol (3.4 mL) was stirred for 6 h at room temperature. The reaction mixture was filtered through a pad of Celite, washing with methanol. The filtrate was neutralized with NaHCO₃ and extracted with DCM (3×5 mL). The combined organic extracts were dried (MgSO₄) and concentrated under vacuum to afford the title compound (60 mg) as a yellow solid. [M+H]⁺ m/z 391.3.

### Example 66

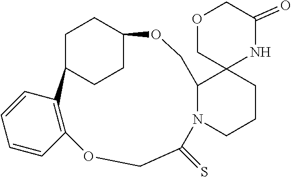 [SMILES: O=C1COCC2(CCCN3C(=S)COc4ccccc4[C@H]4CC[C@H](CC4)OCC32)N1]

### Rel-(1′s,3S,16′R,19′s)-10′-sulfanylidene-8′,18′-dioxa-11′-azaspiro[morpholine-3,15′-tetracyclo[17.2.2.0^{2,7}0.0^{11,16}]tricosane]-2′(7′),3′,5′-trien-5-one

To a solution of Intermediate 171 (60 mg) in THF (0.7 mL) at 0° C. was added dipotassium carbonate (63 mg, 0.456 mmol) then water (0.7 mL). To this mixture 2-chloroacetyl chloride (19 mg, 0.168 mmol) was added dropwise at 0° C. The reaction mixture was stirred at 0° C. for 1 h. The mixture was quenched with water and extracted with DCM (3×15 mL). The combined organic extracts were washed with brine (10 mL), dried (MgSO₄), filtered and concentrated to give an oily residue. The oily residue was dissolved in DCM (1.4 mL) and IPA (2 mL) and cooled to 0° C. Potassium 2-methylpropan-2-olate (69 mg, 0.615 mmol) was added and the reaction mixture was stirred at 0° C. for 1 h. The reaction was quenched by addition of water (3 mL). The mixture was poured onto aqueous saturated NaHCO₃ (5 mL). After extraction with DCM (3×15 mL), the combined organic extracts were washed with brine (10 mL), dried (MgSO₄), filtered and concentrated to give the crude material. The crude material was purified by basic reverse phase column chromatography (20-60% acetonitrile in water (0.1% ammonia)) to afford the title compound (4.5 mg) as a white solid.

LCMS (Method A): [M+H]⁺ m/z 433.5, RT 3.34 minutes

¹H NMR (500 MHZ, CDCl₃) δ 7.19-7.12 (m, 1H), 7.09-7.06 (m, 1H), 6.96 (s, 1H), 6.93-6.79 (m, 1H), 6.69 (d, J=8.0 Hz, 1H), 6.38 (dd, J=9.0, 4.3 Hz, 1H), 5.32 (d, J=11.6 Hz, 1H), 5.03 (d, J=11.6 Hz, 1H), 4.32 (d, J=17.0 Hz, 1H), 4.20-4.02 (m, 3H), 3.91 (t, J=9.0 Hz, 1H), 3.85-3.68 (m, 3H), 3.44 (d, J=11.8 Hz, 1H), 2.60-2.47 (m, 2H), 2.40-2.21 (m, 1H), 2.08-2.02 (m, 1H), 1.94-1.86 (m, 2H), 1.85-1.81 (m, 1H), 1.72-1.57 (m, 2H), 1.51 (s, 1H), 1.45-1.31 (m, 3H).

### Intermediate 172

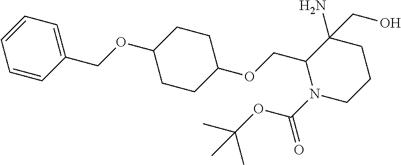 [SMILES: CC(C)(C)OC(=O)N1CCCC(N)(CO)C1COC1CCC(OCc2ccccc2)CC1]

### tert-butyl-rel-(2R,3S)-3-amino-2-({[4-(benzyloxy)cyclohexyl]oxy}methyl)-3-(hydroxymethyl)piperidine-1-carboxylate

Intermediate 172 was prepared using commercially available starting material 1-tert-butyl 4-ethyl 3-oxopiperidine-1,4-dicarboxylate following a similar procedure as described for examples 15a and 15b to afford the title compound (10.5 g) as a colorless oil. [M+H]⁺ m/z 449.4

### Intermediate 173

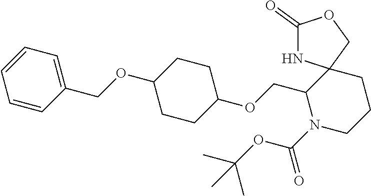 [SMILES: CC(C)(C)OC(=O)N1CCCC2(COC(=O)N2)C1COC1CCC(OCc2ccccc2)CC1]

### tert-butyl-rel-(5R,6S)-6-({[4-(benzyloxy)cyclohexyl]oxy}methyl)-2-oxo-3-oxa-1,7-diazaspiro[4.5]decane-7-carboxylate

Di-1H-imidazol-1-ylmethanone (1.30 g, 8.02 mmol) was added to a stirred solution of Intermediate 172 (3.00 g) and triethylamine (2.8 mL, 20.1 mmol) in anhydrous THF (20 mL) at room temperature and the mixture was stirred for 16 h. The reaction mixture was cooled to room temperature, quenched with a solution of saturated aqueous NH₄Cl (20 mL) and extracted with ethyl acetate (3×25 mL). The combined organic extracts were washed with brine (20 mL), dried over MgSO₄, filtered and concentrated in vacuo to afford the crude material. The crude material was purified by column chromatography (0-100% EtOAc in heptane) to afford the title compound (1.30 g) as a colorless oil. [M+Na]⁺m/z 497.3

### Examples 67a and 67b

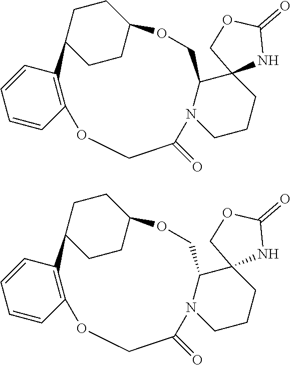

### Example 67a: (1′s,3S,16′R,19′s)-8′,18′-dioxa-11′-azaspiro[1,4-oxazolidine-3,15′-tetracyclo[17.2.2.0^{2,7}0.0^{11,16}]tricosane]-2′(7′),3′,5′-triene-5,10′-dione

### Example 67b: (1′s,3R,16′S,19′s)-8′,18′-dioxa-11′-azaspiro[1,4-oxazolidine-3,15′-tetracyclo[17.2.2.0^{2,7}0.0^{11,16}]tricosane]-2′(7′),3′,5′-triene-5,10′-dione

Examples 67a and 67b were prepared using Intermediate 173 following a similar procedure as described for examples 15a and 15b. The crude material (150 mg) was subjected to chiral preparative purification using Waters 600 eluting with 85/15% v/v n-Hexane/Ethanol, Chiralcel OD-H (20×2.5 cm), 5 μm, flow rate 18 mL/minute to afford the title compounds (Peak 1, 30.6 mg, 100% ee; and Peak 2, 22.5 mg, 98% ee).

### Example 67a: Peak 1 (Stereochemistry Tentatively Assigned)

LCMS (Method B): [M+H]⁺ m/z 401.3, RT 2.85 minutes.

Chiral analysis (Chiralcel OD-H, 20×250 mm, 5 μm, 85:15 n-Hexane: Ethanol): RT 17.6 minutes

¹H NMR (400 MHZ, CDCl₃) δ 7.16 (td, J=7.8, 1.7 Hz, 1H), 7.09 (dd, J=7.4, 1.7 Hz, 1H), 6.93-6.85 (m, 1H), 6.74 (d, J=8.0 Hz, 1H), 5.56 (s, 1H), 5.14-5.05 (m, 2H), 4.43 (d, J=8.6 Hz, 1H), 4.31 (d, J=10.6 Hz, 1H), 4.18 (d, J=8.7 Hz, 1H), 3.88-3.79 (m, 1H), 3.75 (s, 1H), 3.72-3.62 (m, 1H), 3.56-3.41 (m, 2H), 2.67-2.49 (m, 2H), 2.27-2.13 (m, 1H), 2.05 (d, J=14.5 Hz, 1H), 1.97-1.80 (m, 4H), 1.47-1.19 (m, 5H).

### Example 67b: Peak 2 (Stereochemistry Tentatively Assigned)

LCMS (Method B): [M+H]⁺ m/z 401.3, RT 2.85 minutes.

Chiral analysis (Chiralcel OD-H, 20×250 mm, 5 μm, 85:15 n-Hexane:Ethanol): RT 24.7 minutes

¹H NMR (400 MHZ, CDCl₃) δ 7.16 (td, J=7.8, 1.7 Hz, 1H), 7.09 (dd, J=7.4, 1.7 Hz, 1H), 6.90 (td, J=7.4, 1.1 Hz, 1H), 6.74 (dd, J=8.0, 0.9 Hz, 1H), 5.94 (s, 1H), 5.14-5.05 (m, 2H), 4.43 (d, J=8.6 Hz, 1H), 4.31 (d, J=10.6 Hz, 1H), 4.17 (d, J=8.7 Hz, 1H), 3.89-3.80 (m, 1H), 3.76 (s, 1H), 3.68 (dd, J=14.2, 4.1 Hz, 1H), 3.57-3.41 (m, 2H), 2.68-2.49 (m, 2H), 2.29-2.14 (m, 1H), 2.06 (d, J=14.3 Hz, 1H), 1.96-1.80 (m, 4H), 1.49-1.25 (m, 5H)

### Examples 68a and 68b

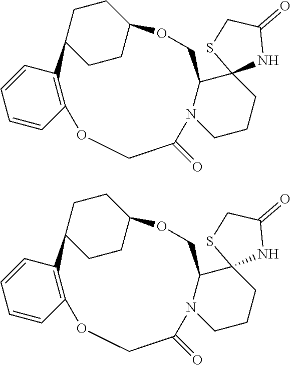

### Example 68a: rel-(1s, 15R,16S,19s)-8, 18-dioxa-11-azaspiro[tetracyclo-[17.2.2.0^{2,7}0.0^{11,16}]tricosane-15,2′-[1,3]thiazolidine]-2 (7),3,5-triene-4′, 10-dione

### Example 68b: rel-(1s,15S,16S,19s)-8, 18-dioxa-11-azaspiro[tetracyclo[17.2.2.0^{2,7}0.0^{11,16}]tricosane-15,2′-[1,3]thiazolidine]-2(7),3,5-triene-4′,10-dione

A solution of diammonium carbonate (17 mg, 0.180 mmol) and Intermediate 166 (100 mg) in toluene (1 mL) was heated to 110° C. for 1 h. 2-Sulfanylacetic acid (0.022 mL, 0.317 mmol) was then added and the reaction mixture was heated for a further 18 hours. The reaction mixture was diluted with water (20 mL) and extracted with EtOAc (3×20 mL). The combined organic extracts were dried over magnesium sulfate and concentrated in vacuo to afford the crude material. The crude material was purified by reverse phase column chromatography (10-100% acetonitrile in water (0.1% formic acid)) to afford the title compounds (Peak 1, 19 mg and Peak 2, 18 mg)

### Example 68a: Peak 1

LCMS (Method B): [M+H]⁺ m/z 417.3, RT 2.90 minutes.

¹H NMR (400 MHZ, DMSO) δ 8.87 (s, 1H), 7.16 (t, J=7.7 Hz, 1H), 7.13-7.04 (m, 1H), 6.92-6.75 (m, 2H), 5.34 (d, J=10.3 Hz, 1H), 4.95 (d, J=7.5 Hz, 1H), 3.97 (d, J=10.3 Hz, 1H), 3.86-3.38 (m, 6H), 2.65-2.52 (m, 2H), 2.27-1.98 (m, 3H), 1.76-1.61 (m, 4H), 1.40-1.11 (m, 4H).

### Example 68b: Peak 2

LCMS (Method B): [M+H]⁺ m/z 417.3, RT 3.36 minutes

¹H NMR (400 MHZ, DMSO) δ 9.20 (s, 1H), 7.19-7.11 (m, 1H), 7.07 (dd, J=7.4, 1.6 Hz, 1H), 6.90 (d, J=7.3 Hz, 1H), 6.84 (t, J=7.4 Hz, 1H), 5.21 (d, J=10.3 Hz, 1H), 4.79 (d, J=7.9 Hz, 1H), 4.01 (d, J=10.3 Hz, 1H), 3.93-3.80 (m, 1H), 3.65 (d, J=6.4 Hz, 2H), 3.60 (d, J=2.8 Hz, 1H), 3.49-3.35 (m, 2H), 2.66-2.53 (m, 2H), 2.28-2.09 (m, 2H), 2.05-1.85 (m, 3H), 1.71-1.52 (m, 2H), 1.45-1.10 (m, 5H).

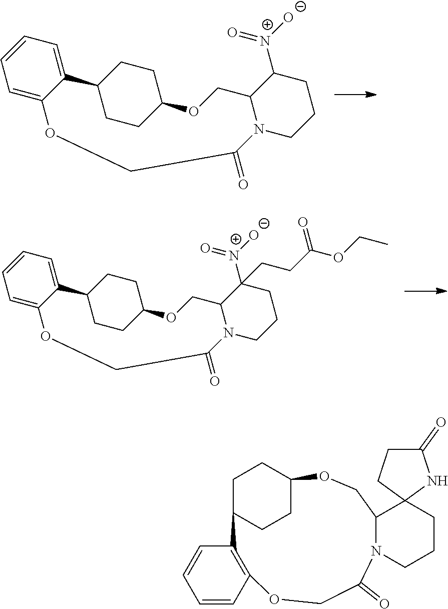 [SMILES: O=C1COc2ccccc2[C@H]2CC[C@H](CC2)OCC2C([N+](=O)[O-])CCCN12 ; CCOC(=O)CCC1([N+](=O)[O-])CCCN2C(=O)COc3ccccc3[C@H]3CC[C@H](CC3)OCC21 ; O=C1CCC2(CCCN3C(=O)COc4ccccc4[C@H]4CC[C@H](CC4)OCC32)N1]

### Intermediate 174

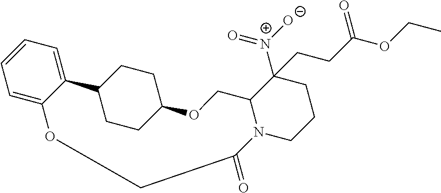

### Ethyl 3-[rel-(1s,15R,16R,19s)-15-nitro-10-oxo-8,18-dioxa-11-azatetracyclo-[17.2.2.0^{2,7}0.0^{11,16}]tricosa-2,4,6-trien-15-yl]propanoate

To a solution of Intermediate 168 (50 mg) and ethyl prop-2-enoate (21 μL, 0.200 mmol) in ethanol (2.5 mL) was added potassium carbonate (37 mg, 0.268 mmol) and the solution was stirred at room temperature for 3 hours. The solids were filtered off and the filtrate was concentrated in vacuo. The residue was suspended in water (10 mL) and extracted with DCM (3×10 mL). The combined organic phases were concentrated in vacuo to afford the title compound (57 mg) as a yellow oil. [M+H]⁺ m/z 475.4

### Example 69

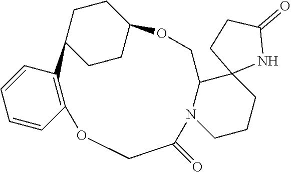 [SMILES: O=C1CCC2(CCCN3C(=O)COc4ccccc4[C@H]4CC[C@H](CC4)OCC32)N1]

### Rel-(1′s,2R,16′R,19′s)-8′,18′-dioxa-11′-azaspiro[pyrrolidine-2,15′-tetracyclo-[17.2.2.0^{2,7}0.0^{11,16}]tricosane]-2′(7′),3′,5′-triene-5,10′-dione

A suspension of Intermediate 174 (57 mg) and zinc (79 mg, 1.21 mmol) in acetic acid (0.36 mL) and ethanol (2.6 mL) was stirred for 16 h at room temperature. The reaction mixture was filtered through a pad of Celite, washing with methanol. The filtrate was neutralized with NaHCO₃ and extracted with DCM (3×10 mL). The combined organic extracts were dried (MgSO₄) and concentrated under vacuum to afford the crude material. The crude material was purified by basic reverse phase column chromatography (20-40% acetonitrile in water (0.1% ammonia)) to afford the title compound (11.3 mg) as a white solid.

LCMS (Method A): [M+H]⁺ m/z 399.3, RT 2.90 minutes

¹H NMR (500 MHz, CDCl₃) δ 7.18-7.13 (m, 1H), 7.11-7.06 (m, 1H), 6.95-6.86 (m, 1H), 6.78-6.71 (m, 1H), 6.58 (s, 1H), 5.11 (d, J=10.5 Hz, 1H), 4.95 (dd, J=10.7, 3.8 Hz, 1H), 4.31 (d, J=10.5 Hz, 1H), 3.93-3.80 (m, 1H), 3.74-3.61 (m, 2H), 3.52-3.36 (m, 2H), 2.84-2.47 (m, 3H), 2.38-2.30 (m, 2H), 2.29-2.16 (m, 1H), 2.08 (d, J=14.4 Hz, 1H), 1.92-1.75 (m, 4H), 1.72-1.65 (m, 2H), 1.54-1.45 (m, 1H), 1.44-1.30 (m, 3H).

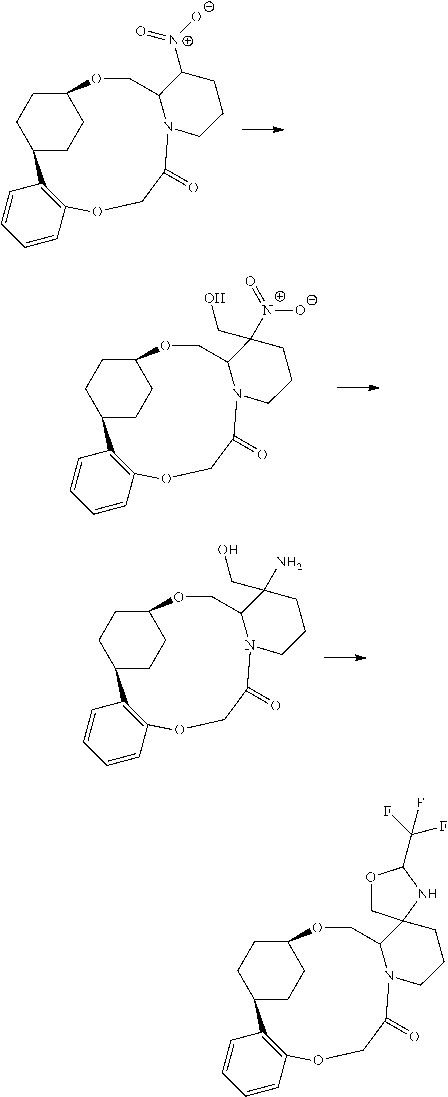 [SMILES: O=C1COc2ccccc2[C@H]2CC[C@H](CC2)OCC2C([N+](=O)[O-])CCCN12 ; O=C1COc2ccccc2[C@H]2CC[C@H](CC2)OCC2N1CCCC2(CO)[N+](=O)[O-] ; NC1(CO)CCCN2C(=O)COc3ccccc3[C@H]3CC[C@H](CC3)OCC21 ; O=C1COc2ccccc2[C@H]2CC[C@H](CC2)OCC2N1CCCC21COC(C(F)(F)F)N1]

### Intermediate 175

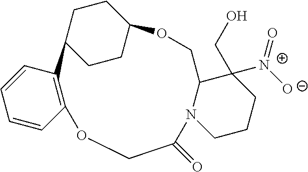 [SMILES: O=C1COc2ccccc2[C@H]2CC[C@H](CC2)OCC2N1CCCC2(CO)[N+](=O)[O-]]

### Rel-(1s, 15S,16R,19s)-15-(hydroxymethyl)-15-nitro-8,18-dioxa-11-azatetracyclo[17.2.2.0^{2,7}0.0^{11,16}]tricosa-2,4,6-trien-10-one

Formaldehyde (37% in water, 0.29 mL, 3.91 mmol) was added to Intermediate 168 (197 mg) and triethylamine (0.073 mL, 0.522 mmol) in THF (2.5 mL) at room temperature. The solution was heated to 70° C. for 18 h. After cooling the reaction mixture was diluted with water (20 mL) and extracted with EtOAc (3×20 mL). The combined organic extracts were washed with brine (20 mL), dried (MgSO₄), filtered and concentrated in vacuo. The crude product was purified by silica gel column chromatography (0-100% EtOAc in heptane) to afford the title compound (118 mg) as a pale yellow solid. [M+Na]⁺ m/z 405.2

### Intermediate 176

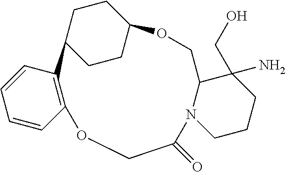 [SMILES: NC1(CO)CCCN2C(=O)COc3ccccc3[C@H]3CC[C@H](CC3)OCC21]

### Rel-(1s, 15S,16R,19s)-15-amino-15-(hydroxymethyl)-8,18-dioxa-11-azatetracyclo[17.2.2.0^{2,7}0.0^{11,16}]tricosa-2,4,6-trien-10-one

A suspension of Intermediate 175 (118 mg) and zinc (148 mg, 2.27 mmol) in acetic acid (0.7 mL) and ethanol (7 mL) was stirred for 18 h at room temperature. Further zinc (148 mg, 2.27 mmol) was added, and the reaction was stirred for 18 hours. The reaction mixture was filtered through a pad of Celite, washing with methanol. The filtrate was neutralized with NaHCO₃ (20 mL) and extracted with DCM (3×20 mL). The combined organic extracts were dried (MgSO₄) and concentrated under vacuum to afford the title compound (80 mg) as an off white solid. [M+H]⁺ m/z 375.3

### Example 70

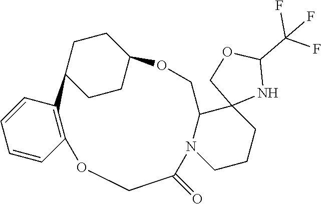 [SMILES: O=C1COc2ccccc2[C@H]2CC[C@H](CC2)OCC2N1CCCC21COC(C(F)(F)F)N1]

### Rel-(1′s,3S,16′R,19′s)-5-(trifluoromethyl)-8′,18′-dioxa-11′-azaspiro[1,4-oxazolidine-3,15′-tetracyclo[17.2.2.0^{2,7}0.0^{11,16}]tricosane]-2′(7′),3′,5′-trien-10′-one

A solution of trifluoroacetaldehyde hydrate (16 mg, 0.101 mmol) in anhydrous THF (0.5 mL) was added dropwise to a stirred solution of Intermediate 176 (40 mg) and titanium (4+) tetraethanolate (39 μL, 0.184 mmol) in anhydrous THF (0.5 mL). The mixture was stirred at 60° C. for 16 h then 110° C. for 18h. Sodium borohydride (10 mg, 0.276 mmol) was added, and the mixture was stirred for 4 h at room temperature. The reaction was quenched with water (10 mL) and diluted with EtOAc (10 mL). The mixture was filtered through Celite. The phases were separated, and the aqueous phase was extracted with further EtOAc (2×10 mL). The combined organic phases were dried over MgSO₄ and concentrated under reduced pressure to afford the crude material. The crude material was purified by reverse phase column chromatography (10-100% acetonitrile in water (0.1% NH₃)) to afford the title compound (4.2 mg) as a white solid.

LCMS (Method A): [M+H]⁺ m/z 455.3, RT 3.99 and 4.09 minutes

¹H NMR (400 MHZ, CDCl₃) δ 7.19-7.13 (m, 1H), 7.09 (dd, J=7.4, 1.5 Hz, 1H), 6.89 (ddd, J=8.4, 5.1, 1.9 Hz, 1H), 6.74 (dd, J=8.0, 2.7 Hz, 1H), 5.15-4.95 (m, 3H), 4.29 (dd, J=10.5, 2.7 Hz, 1H), 4.10 (dd, J=32.9, 8.1 Hz, 1H), 3.93-3.78 (m, 1H), 3.75-3.60 (m, 4H), 3.58-3.45 (m, 1H), 2.75-2.49 (m, 2H), 2.35-2.13 (m, 2H), 2.08 (d, J=11.4 Hz, 1H), 2.00-1.77 (m, 3H), 1.77-1.65 (m, 1H), 1.52-1.27 (m, 5H).

### Intermediate 177

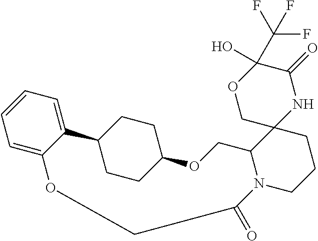 [SMILES: O=C1COc2ccccc2[C@H]2CC[C@H](CC2)OCC2N1CCCC21COC(O)(C(F)(F)F)C(=O)N1]

### Rel-(1′s,3S,16′R,19′s)-6-hydroxy-6-(trifluoromethyl)-8′,18′-dioxa-11′-azaspiro[morpholine-3, 15′-tetracyclo[17.2.2.0^{2,7}0.0^{11,16}]tricosane]-2′,4′,6′-triene-5,10′-dione

Ethyl 3,3,3-trifluoro-2-oxopropanoate (23 mg, 0.133 mmol) was added to a stirred solution of Intermediate 176 (50 mg) in 1,4-dioxane (1.5 mL). The solution was stirred at 110° C. for 16 h. The reaction mixture was cooled to room temperature and the solvent evaporated to afford the crude material. The crude material was purified by column chromatography (0-100% EtOAc in heptane) to afford the title compound (60 mg) as a colorless oil. [M+H]⁺ m/z 516.4

### Examples 71a and 71b

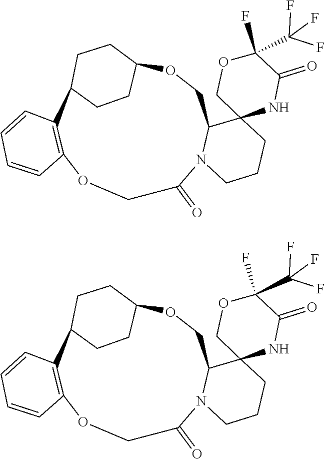 [SMILES: O=C1COc2ccccc2[C@H]2CC[C@H](CC2)OC[C@@H]2N1CCC[C@@]21CO[C@](F)(C(F)(F)F)C(=O)N1 ; O=C1COc2ccccc2[C@H]2CC[C@H](CC2)OC[C@@H]2N1CCC[C@@]21CO[C@@](F)(C(F)(F)F)C(=O)N1]

### Example 71a: rel-(1′s,3S,16′R,19′s)-6 (R)-fluoro-6-(trifluoromethyl)-8′,18′-dioxa-11′-azaspiro[morpholine-3,15′-tetracyclo[17.2.2.0^{2,7}0.0^{11,16}]tricosane]-2′(7′),3′,5′-triene-5,10′-dione

### Example 71b: rel-(1′s,3R,16′S,19′s)-6 (S)-fluoro-6-(trifluoromethyl)-8′,18′-dioxa-11′-azaspiro[morpholine-3,15′-tetracyclo[17.2.2.0^{2,7}0.0^{11,16}]tricosane]-2′(7′),3′,5′-triene-5,10′-dione

DAST (19 μL, 0.140 mmol) was added to a solution of Intermediate 177 (40 mg) in anhydrous DCM (0.9 mL) at 0° C. The reaction mixture was stirred at 0° C. for 2 hours and ice and saturated aqueous NaHCO₃were poured into the mixture. The reaction mixture was extracted with DCM (2×10 mL). The combined organic phases were washed with brine, dried over MgSO₄ and concentrated in vacuo to afford the crude. The crude material was purified by basic reverse phase column chromatography (15-45% acetonitrile in water (0.1% ammonia)) to afford the title compounds (Peak 1, 3.9 mg and Peak 2, 3.8 mg).

### Peak 1: Example 71a

LCMS (Method A): [M+H]⁺ m/z 518.4, RT 3.58 minutes

¹H NMR (500 MHZ, CDCl₃) δ 8.08 (s, 1H), 7.20-7.14 (m, 1H), 7.13-7.08 (m, 1H), 6.94-6.88 (m, 1H), 6.77-6.73 (m, 1H), 5.41 (dd, J=10.2, 4.1 Hz, 1H), 5.10 (d, J=10.6 Hz, 1H), 4.38 (d, J=11.8 Hz, 1H), 4.32 (d, J=10.6 Hz, 1H), 3.92-3.83 (m, 2H), 3.76-3.70 (m, 1H), 3.67 (s, 1H), 3.58-3.49 (m, 1H), 3.40 (dd, J=8.8, 4.2 Hz, 1H), 2.66-2.52 (m, 2H), 2.27-2.17 (m, 1H), 2.09-2.02 (m, 1H), 1.99-1.93 (m, 1H), 1.90 (d, J=13.2 Hz, 1H), 1.86-1.77 (m, 2H), 1.56-1.44 (m, 2H), 1.43-1.31 (m, 3H).

### Peak 2: Example 71b

LCMS (Method A): [M+H]⁺ m/z 518.4, RT 3.76 minutes

¹H NMR (500 MHZ, CDCl₃) δ 7.78 (s, 1H), 7.17 (t, J=7.0 Hz, 1H), 7.12-7.04 (m, 1H), 6.96-6.84 (m, 1H), 6.74 (d, J=8.3 Hz, 1H), 5.39-4.85 (m, 2H), 4.72-4.45 (m, 1H), 4.38-4.19 (m, 1H), 4.00-3.93 (m, 1H), 3.94-3.42 (m, 6H), 2.67-2.51 (m, 2H), 2.34-1.73 (m, 7H), 1.43-1.30 (m, 3H).

Below examples were prepared following similar procedures as described for Example 7 using the appropriate reagents

| | | | Obs. | |
|---|---|---|---|---|
| Ex. | Structure | Name | Mass | ¹H NMR |
| 72 | 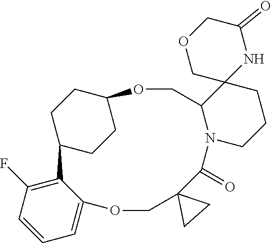 [SMILES: O=C1COCC2(CCCN3C(=O)C4(CC4)COc4cccc(F)c4[C@H]4CC[C@H](CC4)OCC32)N1] | (1′s,20′s)-3′- fluorodispiro [cyclopropane-1,10′- [8,19]dioxa- [12]azatetracyclo [18.2.2.0²,⁷.0¹²,¹⁷] tetracosane-16′,3″- morpholine]- 2′,4′,6′-triene-5″,11′- dione | LCMS (Method A): [M + H]⁺ m/z 473.4, RT 3.08 minutes | ¹H NMR (400 MHz, CDCl₃) δ 7.06 (td, J = 8.2, 6.5 Hz, 1H), 6.73 (td, J = 8.3, 1.2 Hz, 2H), 6.07 (s, 1H), 5.32(dd, J = 9.3, 4.0 Hz, 1H), 4.52 (dd, J = 14.2, 4.5 Hz,1H), 4.26 (d, J = 16.9 Hz, 1H), 4.21 − 4.14 (m, 1H),4.08 (d, J = 16.9 Hz, 1H), 3.92 − 3.77 (m, 3H), 3.74 −3.64 (m, 2H), 3.40 (td, J = 13.6, 3.1 Hz, 1H), 3.32 (d, J = 11.7 Hz, 1H), 3.06 (ddt, J = 12.5, 7.6, 3.9 Hz, 1H),2.40 − 2.26 (m, 1H), 2.26 − 2.06 (m, 1H), 2.06 − 1.97(m, 1H), 1.84 − 1.69 (m, 2H), 1.54 − 1.23 (m, 8H), 1.14-1.04 (m, 1H), 1.04 − 0.95 (m, 1H), 0.86 − 0.77 (m,1H). |
| 72a | 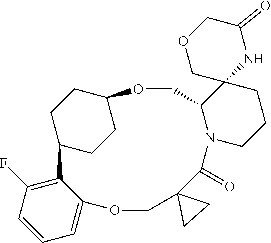 [SMILES: O=C1COC[C@]2(CCCN3C(=O)C4(CC4)COc4cccc(F)c4[C@H]4CC[C@H](CC4)OC[C@@H]32)N1] | (1′s,16′R,17′S,20′s)- 3′- fluorodispiro [cyclopropane-1,10′- [8,19]dioxa- [12]azatetracyclo [18.2.2.0²,⁷.0¹²,¹⁷] tetracosane-16′,3″- morpholine]- 2′,4′,6′-triene-5″,11′- dione | LCMS (Method C): [M + H]⁺ m/z 473.2, RT 0.94 minutes | ¹H NMR (600 MHz, CD₃OD) δ 7.10 (td, J = 8.2, 6.8 Hz, 1H), 6.88 (d, J = 8.2 Hz, 1H), 6.72 (t, J = 9.0 Hz, 1H), 5.40 (dd, J = 11.4, 3.8 Hz, 1H), 4.48 (br dd, J = 14.0, 3.1 Hz, 1H), 4.18 (d, J = 16.8 Hz, 1H), 4.12 (d, J = 10.2 Hz, 1H), 4.05 (d, J = 16.8 Hz, 1H), 4.03 − 3.98 (m, 1H), 3.94 (d, J = 10.0 Hz, 1H), 3.89 (d, J = 11.9 Hz, 1H), 3.70 (br s, 1H), 3.48 (dd, J = 9.1, 4.0 Hz, 1H), 3.38 − 3.32 (m, 1H), 3.33 − 3.30 (m, 1H), 3.06 (tt, J = 12.5, 3.9 Hz, 1H), 2.44 (qd, J = 12.9, 3.5 Hz, 1H), 2.33 − 2.25 (m, 1H), 2.26 − 2.20 (m, 1H), 1.95 − 1.86 (m, 2H), 1.71 (br d, J = 9.9 Hz, 2H), 1.63 − 1.55 (m, |
| | | | | 1H), 1.51 (tdd, J = 13.4, 4.0, 2.1 Hz, |
| | | | | 1H), 1.45 − 1.39 (m, 1H), 1.39 − 1.32 |
| | | | | (m, 1H), 1.30 − 1.23 (m, 1H), 1.19 − |
| | | | | 1.10 (m, 3H), 0.93 − 0.87 (m, 1H). |
| 72b | 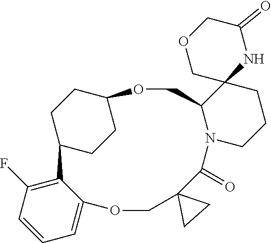 [SMILES: O=C1COC[C@@]2(CCCN3C(=O)C4(CC4)COc4cccc(F)c4[C@H]4CC[C@H](CC4)OC[C@H]32)N1] | (1′s,16′S,17′R,20′s)- 3′- fluorodispiro[cyclo propane-1,10′- [8,19]dioxa- [12]azatetracyclo [18.2.2.0²,⁷.0¹²,¹⁷] tetracosane-16′,3″- morpholine]- 2′,4′,6′-triene-5″,11′- dione | LCMS (Method C): [M + H]⁺ m/z 473.2, RT 0.94 minutes | ¹H NMR (600 MHz, CD₃OD) δ 7.10 (td, J = 8.2, 6.8 Hz, 1H), 6.88 (d, J = 8.2 Hz, 1H), 6.72 (t, J = 9.0 Hz, 1H), 5.40 (dd, J = 11.4, 3.8 Hz, 1H), 4.48 (br dd, J = 14.0, 3.1 Hz, 1H), 4.18 (d, J = 16.8 Hz, 1H), 4.12 (d, J = 10.2 Hz, 1H), 4.05 (d, J = 16.8 Hz, 1H), 4.03 − 3.98 (m, 1H), 3.94 (d, J = 10.0 Hz, 1H), 3.89 (d, J = 11.9 Hz, 1H), 3.70 (br s, 1H), 3.48 (dd, J = 9.1, 4.0 Hz, 1H), 3.38 − 3.32 (m, 1H), 3.33 − 3.30 (m, 1H), 3.06 (tt, J = 12.5, 3.9 Hz, 1H), 2.44 (qd, J = 12.9, 3.5 Hz, 1H), 2.33 − 2.25 (m, 1H), 2.26 − 2.20 (m, 1H), 1.95 − 1.86 (m, 2H), 1.71 (br d, J = 9.9 Hz, 2H), 1.63 − 1.55 (m, |
| | | | | 1H), 1.51 (tdd, J = 13.4, 4.0, 2.1 Hz, |
| | | | | 1H), 1.45 − 1.39 (m, 1H), 1.39 − 1.32 |
| | | | | (m, 1H), 1.30 − 1.23 (m, 1H), 1.19 − |
| | | | | 1.10 (m, 3H), 0.93 − 0.87 (m, 1H). |
| 73a | 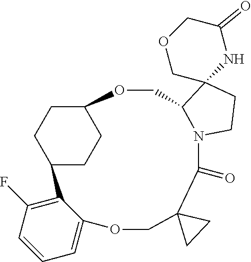 [SMILES: O=C1COC[C@]2(CCN3C(=O)C4(CC4)COc4cccc(F)c4[C@H]4CC[C@H](CC4)OC[C@@H]32)N1] | (1′s,15′R,16′S,19′s)- 3′- fluorodispiro [cyclopropane-1,10′- [8,18]dioxa- [12]azatetracyclo[17. 2.2.0²,⁷.0¹²,¹⁶] tricosane-15′,3″- morpholine]- 2′,4′,6′-triene-5″,11′- dione | LCMS (Method C): [M + H]⁺ m/z 459.4, RT 0.97 minutes | ¹H NMR (500 MHz, CDCl₃) δ 7.00 − 7.13 (m, 1H), 6.60 − 6.88 (m, 2H), 6.31 − 6.46 (m, 1H), 4.41 − 4.58 (m, 1H), 4.27 − 4.40 (m, 1H), 4.26 − 4.37 (m, 1H), 4.16 − 4.27 (m, 3H), 3.83 (br s, 1H), 3.64 − 3.77 (m, 3H), 3.53 − 3.62 (m, 1H), 3.32 (br d, J = 9.6 Hz, 1H), 2.97 − 3.21 (m, 1H), 2.39 − 2.49 (m, 1H), 2.27 − 2.39 (m, 1H), 2.18 − 2.26 (m, 1H), 2.05 − 2.13 (m, 1H), 1.95 − 2.08 (m, 1H), 1.80 − 1.89 (m, 1H), 1.18 − 1.72 (m, 4H), 1.04 − 1.26 (m, 2H), 0.58 − 1.00 (m, 2H). |
| 73b | 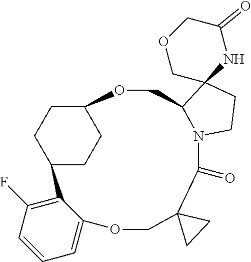 [SMILES: O=C1COC[C@@]2(CCN3C(=O)C4(CC4)COc4cccc(F)c4[C@H]4CC[C@H](CC4)OC[C@H]32)N1] | (1′s,15′S,16′R,19′s)- 3′- fluorodispiro [cyclopropane-1,10′- [8,18]dioxa- [12]azatetracyclo [17.2.2.0²,⁷.0¹²,¹⁶] tricosane-15′,3″- morpholine]- 2′,4′,6′-triene-5″,11′- dione | LCMS (Method C): [M + H]⁺ m/z 459.5, RT 0.97 minutes | ¹H NMR (500 MHz, CDCl₃) δ 7.00 − 7.13 (m, 1H), 6.60 − 6.88 (m, 2H), 6.31 − 6.46 (m, 1H), 4.41 − 4.58 (m, 1H), 4.27 − 4.40 (m, 1H), 4.26 − 4.37 (m, 1H), 4.16 − 4.27 (m, 3H), 3.83 (br s, 1H), 3.64 − 3.77 (m, 3H), 3.53 − 3.62 (m, 1H), 3.32 (br d, J = 9.6 Hz, 1H), 2.97 − 3.21 (m, 1H), 2.39 − 2.49 (m, 1H), 2.27 − 2.39 (m, 1H), 1.18 − 1.72 (m, 4H), 1.04 − 1.26 (m, 1H), 1.95 − 2.08 (m, 1H), 1.80 − 1.89 (m, 1H), 2.18 − 2.26 (m, 1H), 2.05 − 2.13 (m, 2H), 0.58 − 1.00 (m, 2H). |

### Examples 74 and 75

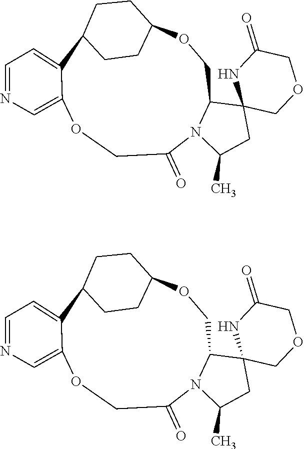 [SMILES: C[C@@H]1C[C@@]2(COCC(=O)N2)[C@@H]2CO[C@H]3CC[C@H](CC3)c3ccncc3OCC(=O)N12 ; C[C@@H]1C[C@]2(COCC(=O)N2)[C@H]2CO[C@H]3CC[C@H](CC3)c3ccncc3OCC(=O)N12]

### Example 74

(1′s,3S, 13′R,16′R,19′s)-13′-methyl-8′,18′-dioxa-5′,12′-diazaspiro[morpholine-3,15′-tetracyclo [17.2.2.0^{2,7}0.0^{12,16}]tricosane]-2′,4′,6′-triene-5,11′-dione

### Example 75

(1′s,3R,13′R,16′S,19′s)-13′-methyl-8′,18′-dioxa-5′,12′-diazaspiro[morpholine-3,15′-tetracyclo[17.2.2.0^{2,7}0.0^{12,16}]tricosane]-2′,4′,6′-triene-5,11′-dione

Examples 74 and 75 were prepared using known starting material 1-tert-butyl 3-ethyl (2R)-2-methyl-4-oxopyrrolidine-1,3-dicarboxylate following a similar procedure as described for Example 8, and then subjected to preparative HPLC: MDAP Waters with mass spectrometry detection (MS: ZQ2000). Column: CSH C18 (30×100 mm, 3-m). Conditions: [A1: Water+0.1% HCOOH]; [B1: MeCN]. Gradient: from 3.0% B1 to 50.0% B1 in 10 min (flow: 40.00 mL/min). Detection: UV/V is detection range 210 nm to 350 nm MS (ES+/ES—) Scan range 100 to 1000 AMU to afford the title compounds (Peak 1, 2 mg and Peak 2, 1.4 mg).

### Peak 1: Example 74

LCMS (Method C): [M+H]⁺ m/z 430.2, RT 0.48 minutes

¹H NMR (500 MHZ, CD₃OD) δ 8.14 (s, 1H), 8.02 (br d, J=4.8 Hz, 1H), 7.10-7.19 (m, 1H), 4.87-4.93 (m, 1H), 4.49 (dd, J=11.0, 2.4 Hz, 1H), 4.10-4.25 (m, 3H), 3.99 (dt, J=8.1, 3.0 Hz, 1H), 3.76 (dd, J=9.4, 2.7 Hz, 1H), 3.70 (br s, 1H), 3.66 (d, J=12.1 Hz, 1H), 3.51-3.62 (m, 2H), 3.46 (d, J=12.1 Hz, 1H), 2.50-2.67 (m, 3H), 2.42-2.48 (m, 1H), 2.26 (dd, J=13.5, 8.3 Hz, 1H), 1.89-2.09 (m, 2H), 1.84 (dd, J=13.6, 9.1 Hz, 1H), 1.36 (d, J=6.3 Hz, 3H), 1.31-1.64 (m, 4H).

### Peak 2: Example 75

LCMS (Method D): [M+H]⁺ m/z 430.1, RT 0.70 minutes

¹H NMR (500 MHZ, CD₃OD) δ 8.23 (s, 1H), 8.01 (d, J=4.8 Hz, 1H), 7.14 (d, J=4.8 Hz, 1H), 4.52 (dt, J=7.9, 3.6 Hz, 1H), 4.39-4.44 (m, 2H), 4.29-4.36 (m, 2H), 4.14 (q, J=16.9 Hz, 2H), 4.04 (d, J=11.5 Hz, 1H), 3.84 (br s, 1H), 3.62 (d, J=11.7 Hz, 1H), 3.23 (ddd, J=15.7, 11.9, 4.1 Hz, 1H), 3.13-3.18 (m, 1H), 2.73 (dd, J=13.0, 9.8 Hz, 1H), 2.51-2.66 (m, 3H), 2.11-2.24 (m, 2H), 1.90-1.98 (m, 1H), 1.82 (d, J=12.9 Hz, 1H), 1.48-1.58 (m, 1H), 1.38 (d, J=6.7 Hz, 3H), 1.31-1.45 (m, 2H), 1.18-1.25 (m, 1H).

### Intermediate 178

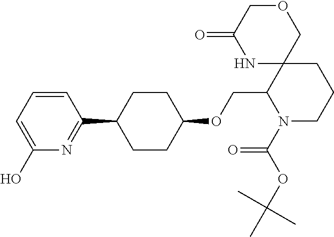 [SMILES: CC(C)(C)OC(=O)N1CCCC2(COCC(=O)N2)C1CO[C@H]1CC[C@@H](c2cccc(O)n2)CC1]

### tert-butyl 2-oxo-7-({[(1s, 4s)-4-(6-hydroxypyridin-2-yl)cyclohexyl]oxy}methyl)-4-oxa-1,8-diazaspiro[5.5]undecane-8-carboxylate

Intermediate 178 was prepared following a similar procedure as described for Intermediate 49 to afford the title compound (150 mg). [M+H]⁺ m/z 476.3

### Intermediate 179

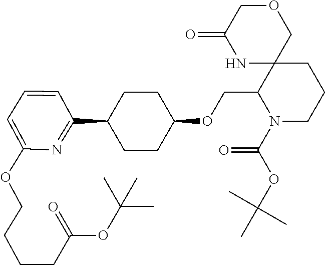 [SMILES: CC(C)(C)OC(=O)CCCCOc1cccc([C@H]2CC[C@@H](OCC3N(C(=O)OC(C)(C)C)CCCC34COCC(=O)N4)CC2)n1]

tert-butyl 2-oxo-7-({[(1s,4s)-4-(6-{[5-(tert-butoxy)-5-oxopentyl]oxy}pyridin-2-yl)cyclohexyl]oxy}methyl)-4-oxa-1,8-diazaspiro[5.5]undecane-8-carboxylate

A suspension of Intermediate 178 (140 mg), tert-butyl 5-bromopentanoate (698 mg, 2.94 mmol) and silver carbonate (162 mg, 0.59 mmol) in toluene (1.5 mL) was stirred at 100° C. for 16 h. The mixture was then cooled to room temperature, diluted with DCM (50 mL) and filtered through a Celite pad. The filtrate was concentrated in vacuo and the residue was purified by column chromatography (0-30% MeCN in DCM) to afford the title compound (141 mg) as a colorless oil. [M+H]⁺ m/z 632.4

### Intermediate 180

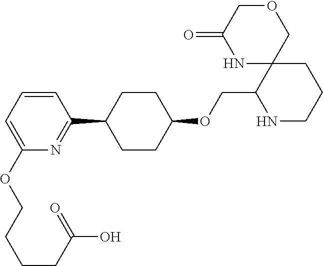 [SMILES: O=C(O)CCCCOc1cccc([C@H]2CC[C@@H](OCC3NCCCC34COCC(=O)N4)CC2)n1]

### 5-({6-[(1s,4s)-4-({2-oxo-4-oxa-1,8-diazaspiro[5.5]undecan-7-yl}methoxy)-cyclohexyl]pyridin-2-yl}oxy) pentanoic acid

Intermediate 180 was prepared following a similar procedure as described for Intermediate 12 to afford the title compound (134 mg). [M+H]⁺ m/z 476.3

### Example 76

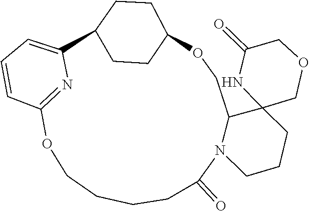 [SMILES: O=C1COCC2(CCCN3C(=O)CCCCOc4cccc(n4)[C@H]4CC[C@H](CC4)OCC32)N1]

### Example 76: (1′s,21′s)-7′,20′-dioxa-13′,26′-diazaspiro[morpholine-3,17′-tetracyclo[19.2.2.1^{2,6}0.0^{13,18}]hexacosane]-2′,4′,6′(26′)-triene-5,12′-dione

Example 76 was prepared following a similar procedure as described for Example 1 to afford the title compound (58 mg).

LCMS (Method C): [M+H]⁺ m/z 458.3, RT 0.96 minutes

¹H NMR (500 MHZ, CD₃OD) δ 7.44 (dd, J=8.2, 7.1 Hz, 1H), 6.65 (d, J=7.1 Hz, 1H), 6.45 (d, J=7.7 Hz, 1H), 4.91 (dt, J=10.8, 7.9 Hz, 1H), 4.60 (dd, J=10.0, 3.2 Hz, 1H), 4.49 (br dd, J=13.9, 4.0 Hz, 1H), 4.23-4.30 (m, 1H), 4.07-4.15 (m, 2H), 3.99-4.06 (m, 1H), 3.85-3.91 (m, 1H), 3.75-3.83 (m, 1H), 3.66 (br s, 1H), 3.35 (t, J=6.0 Hz, 1H), 2.88-3.01 (m, 1H), 2.71-2.81 (m, 1H), 2.61-2.73 (m, 1H), 2.55-2.63 (m, 1H), 1.31-2.32 (m, 16H).

### Examples 76a and 76b

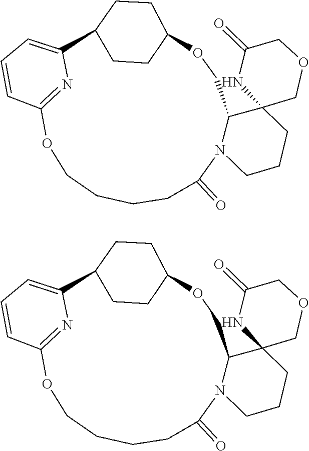

### Example 76a: (1′s,3R,18′S,21′s)-7′,20′-dioxa-13′,26′-diazaspiro[morpholine-3,17′-tetracyclo[19.2.2.1^{2,6}0.0^{13,18}]hexacosane]-2′,4′,6′(26′)-triene-5,12′-dione

### Example 76b: (1′s,3S,18′R,21′s)-7′,20′-dioxa-13′,26′-diazaspiro[morpholine-3,17′-tetracyclo[19.2.2.1^{2,6}0.0^{13,18}]hexacosane]-2′,4′,6′(26′)-triene-5,12′-dione

Example 76 (56 mg) was subjected to chiral preparative SFC purification using as modifier 30% methanol+0.1% isopropylamine, Chiralpak IC (25×2.0 cm), 5 μm, flow rate 45 mL/minute, pressure 120 bar, temperature 40° C., UV detection 220 nm, loop 500 μL, to afford the title compounds (Peak 1, 16.3 mg, 100% ee; and Peak 2, 17.9 mg, 99.7% ee).

Peak 1: Example 76a (Stereochemistry tentatively assigned)

LCMS (Method C): [M+H]⁺ m/z 458.3, RT 0.97 minutes

Chiral SFC analysis (Chiralpak IC (25×0.46 cm), 5u; modifier (methanol+0.1% isopropylamine) 30% v/v; flowrate 2.5 mL/min; pressure 120 bar; temp. 38° C.; UV detection 220 nm; loop 20 μL): RT 9.8 minutes

¹H NMR (500 MHZ, CD₃OD) δ 7.44 (dd, J=8.2, 7.1 Hz, 1H), 6.65 (d, J=7.1 Hz, 1H), 6.45 (d, J=7.7 Hz, 1H), 4.91 (dt, J=10.8, 7.9 Hz, 1H), 4.60 (dd, J=10.0, 3.2 Hz, 1H), 4.49 (br dd, J=13.9, 4.0 Hz, 1H), 4.23-4.30 (m, 1H), 4.07-4.15 (m, 2H), 3.99-4.06 (m, 1H), 3.85-3.91 (m, 1H), 3.75-3.83 (m, 1H), 3.66 (br s, 1H), 3.35 (t, J=6.0 Hz, 1H), 2.88-3.01 (m, 1H), 2.71-2.81 (m, 1H), 2.61-2.73 (m, 1H), 2.55-2.63 (m, 1H), 1.31-2.32 (m, 16H).

Peak 2: Example 76b (Stereochemistry tentatively assigned)

LCMS (Method C): [M+H]⁺ m/z 458.3, RT 0.97 minutes

Chiral SFC analysis (Chiralpak IC (25×0.46 cm), 5u; modifier (methanol+0.1% isopropylamine) 30% v/v; flowrate 2.5 mL/min; pressure 120 bar; temp. 38C; UV detection 220 nm; loop 20 μL): RT 13.9 minutes.

¹H NMR (500 MHZ, CD₃OD) δ 7.44 (dd, J=8.2, 7.1 Hz, 1H), 6.65 (d, J=7.1 Hz, 1H), 6.45 (d, J=7.7 Hz, 1H), 4.91 (dt, J=10.8, 7.9 Hz, 1H), 4.60 (dd, J=10.0, 3.2 Hz, 1H), 4.49 (br dd, J=13.9, 4.0 Hz, 1H), 4.23-4.30 (m, 1H), 4.07-4.15 (m, 2H), 3.99-4.06 (m, 1H), 3.85-3.91 (m, 1H), 3.75-3.83 (m, 1H), 3.66 (br s, 1H), 3.35 (t, J=6.0 Hz, 1H), 2.88-3.01 (m, 1H), 2.71-2.81 (m, 1H), 2.61-2.73 (m, 1H), 2.55-2.63 (m, 1H), 1.31-2.32 (m, 16H).

### Intermediate 181

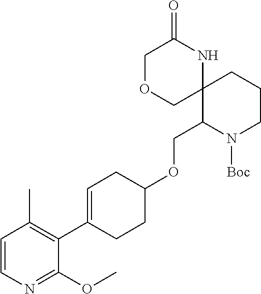 [SMILES: COc1nccc(C)c1C1=CCC(OCC2N(C(=O)OC(C)(C)C)CCCC23COCC(=O)N3)CC1]

### tert-butyl 7-({[4-(2-methoxy-4-methylpyridin-3-yl)cyclohex-3-en-1-yl]oxy}methyl)-2-oxo-4-oxa-1,8-diazaspiro[5.5]undecane-8-carboxylate

Intermediate 181 was prepared following a similar procedure as described for Intermediate 48 to afford the title compound (1.38 g) as a colorless oil. [M+H]⁺ m/z 502.3

### Intermediate 182

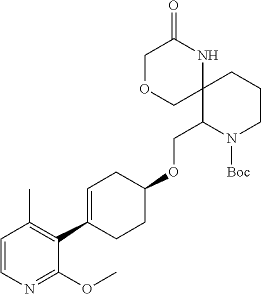

### tert-butyl 2-oxo-7-({[(1s,4s)-4-(2-methoxy-4-methylpyridin-3-yl)cyclohexyl]-oxy}methyl)-4-oxa-1,8-diazaspiro[5.5]undecane-8-carboxylate

Intermediate 182 was prepared following a similar procedure as described for Intermediate 8 to afford the title compound (1.02 g) as a colorless oil. [M+H]⁺ m/z 504.5

### Intermediate 183

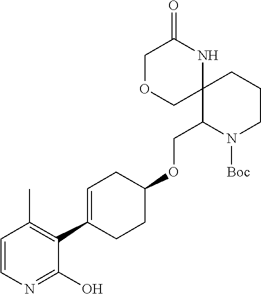

### tert-butyl 2-oxo-7-({[(1s,4s)-4-(2-hydroxy-4-methylpyridin-3-yl)cyclohexyl]oxy}methyl)-4-oxa-1,8-diazaspiro[5.5]undecane-8-carboxylate

To a mixture of Intermediate 182 (980 mg) and acetic acid (19 mL), KI (1.94 g, 11.68 mmol) was added. The suspension was stirred at 70° C. for 18 h. Sat. aq. NaHCO₃ (200 mL) was added and the mixture was extracted with EtOAc (3×50 mL). The combined organic extracts were washed with water (100 mL) and 0.1 M aq. Na₂SO₃ (50 mL), dried (Na₂SO₄) and evaporated in vacuo to afford the title compound (692 mg) as a yellow oil. [M+H]⁺ m/z 490.5

### Intermediate 184

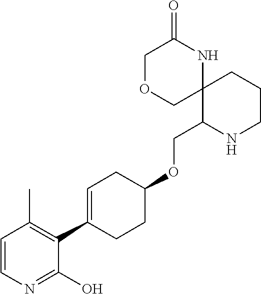

### 7-({[(1s,4s)-4-(2-hydroxy-4-methylpyridin-3-yl)cyclohexyl]oxy}methyl)-4-oxa-1,8-diazaspiro[5.5]undecan-2-one

A 3 M solution of hydrogen chloride in CPME (10.2 mL, 30.6 mmol) was added to Intermediate 183 (300 mg) in MeCN (2.7 mL) at room temperature and the reaction mixture was stirred for 30 minutes. The reaction mixture was concentrated in vacuo and co-evaporated twice with toluene to remove all the excess HCl to afford the title compound (350 mg) as a light brown solid. [M+H]⁺ m/z 390.5

### Intermediate 185

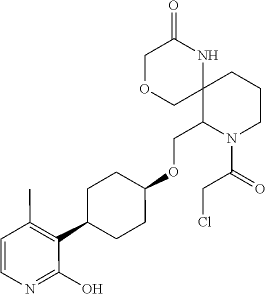 [SMILES: Cc1ccnc(O)c1[C@H]1CC[C@@H](OCC2N(C(=O)CCl)CCCC23COCC(=O)N3)CC1]

### 8-(2-chloroacetyl)-7-({[(1s,4s)-4-(2-hydroxy-4-methylpyridin-3-yl)cyclohexyl]oxy}methyl)-4-oxa-1,8-diazaspiro[5.5]undecan-2-one

To a mixture of Intermediate 184 (350 mg) and DCM (8 mL), was added triethylamine (0.35 mL, 2.5 mmol) and 2-chloroacetyl chloride (0.05 mL, 0.62 mmol). The mixture was stirred under N₂ (g) for 25 h at room temperature. The mixture was diluted with EtOAc (50 mL) and washed with water (50 mL) and sat. aq. NaHCO₃ (50 mL). The organic layer was dried (Na₂SO₄) and concentrated in vacuo to afford the title compound (350 mg) as an off-white solid. [M+H]⁺ m/z 466.5

### Example 77

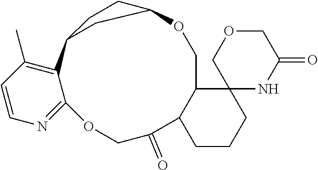 [SMILES: Cc1ccnc2c1[C@H]1CC[C@H](C1)OCC1C(CCCC13COCC(=O)N3)C(=O)CO2]

### (1′s,19′s)-3′-methyl-8′,18′-dioxa-6′,11′-diazaspiro[morpholine-3,15′-tetracyclo[17.2.2.0^{2,7}0.0^{11,16}]tricosane]-2′,4′,6′-triene-5,10′-dione

A mixture of Intermediate 185 (300 mg) and potassium carbonate (236 mg, 1.71 mmol) in MeCN (312 mL) was stirred under nitrogen at 90° C. for 12 h. The mixture was filtered through Celite and concentrated in vacuo. The crude was purified by silica gel column chromatography (0-100% EtOAc/EtOH 9:1 in cyclohexane) and then by reverse phase column chromatography (2-70% acetonitrile in water (0.1% formic acid)) to afford the title compound (26 mg) as a white solid. LCMS (Method A): [M+H]⁺ m/z 430.5, RT 0.80 minutes

¹H NMR (400 MHZ, CD₃OD) δ 7.89-7.72 (m, 1H), 6.85-6.75 (m, 1H), 5.35 (dd, J=4.1, 11.3 Hz, 1H), 5.30-5.23 (m, 1H), 4.37 (d, J=11.3 Hz, 1H), 4.27-3.97 (m, 4H), 3.90-3.70 (m, 2H), 3.54-3.43 (m, 2H), 3.39 (d, J=11.7 Hz, 1H), 3.12-2.96 (m, 1H), 2.74-2.57 (m, 1H), 2.32 (s, 3H), 2.42-2.28 (m, 1H), 2.21 (br d, J=11.7 Hz, 1H), 2.08-1.25 (m, 8H), 1.24-1.13 (m, 1H).

### Examples 77a and 77b

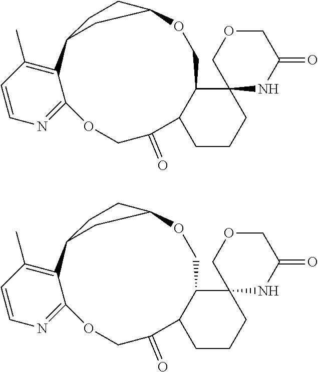 [SMILES: Cc1ccnc2c1[C@H]1CC[C@H](C1)OC[C@@H]1C(CCC[C@@]13COCC(=O)N3)C(=O)CO2 ; Cc1ccnc2c1[C@H]1CC[C@H](C1)OC[C@H]1C(CCC[C@]13COCC(=O)N3)C(=O)CO2]

### Example 77a: (1′s,3S,16′R,19′s)-3′-methyl-8′,18′-dioxa-6′,11′-diazaspiro[morpholine-3,15′-tetracyclo[17.2.2.0^{2,7}0.0^{11,16}]tricosane]-2′,4′,6′-triene-5,10′-dione

### Example 77b: (1′s,3R,16′S,19′s)-3′-methyl-8′,18′-dioxa-6′, 11′-diazaspiro[morpholine-3,15′-tetracyclo[17.2.2.0^{2,7}0.0^{11,16}]tricosane]-2′,4′,6′-triene-5,10′-dione

Example 77 (22 mg) was subjected to chiral preparative SFC purification using as modifier 15% methanol+0.1% isopropylamine, Chiralpak OD-H (25×2.0 cm), 5 μm, flow rate 45 mL/minute, pressure 120 bar, temperature 40° C., UV detection 220 nm, loop 500 μL, to afford the title compounds (Peak 1, 8 mg, 96% ee; and Peak 2, 4.8 mg, 100% ee).

### Peak 1: Example 77a (Stereochemistry Tentatively Assigned)

LCMS (Method C): [M+H]⁺ m/z 430.2, RT 0.79 minutes

Chiral SFC analysis (Chiralpak OD-H (25×0.46 cm), 5u; modifier (methanol+0.1% isopropylamine) 15% v/v; flowrate 2.5 mL/min; pressure 120 bar; temp. 38° C.; UV detection 220 nm; loop 25 μL): RT 14.9 minutes

¹H NMR (400 MHZ, CD₃OD) δ 7.89-7.72 (m, 1H), 6.85-6.75 (m, 1H), 5.35 (dd, J=4.1, 11.3 Hz, 1H), 5.30-5.23 (m, 1H), 4.37 (d, J=11.3 Hz, 1H), 4.27-3.97 (m, 4H), 3.90-3.70 (m, 2H), 3.54-3.43 (m, 2H), 3.39 (d, J=11.7 Hz, 1H), 3.12-2.96 (m, 1H), 2.74-2.57 (m, 1H), 2.32 (s, 3H), 2.42-2.28 (m, 1H), 2.21 (br d, J=11.7 Hz, 1H), 2.08-1.25 (m, 8H), 1.24-1.13 (m, 1H).

### Peak 1: Example 77b (Stereochemistry Tentatively Assigned)

LCMS (Method C): [M+H]⁺ m/z 430.2, RT 0.79 minutes

Chiral SFC analysis (Chiralpak OD-H (25×0.46 cm), 5u; modifier (methanol+0.1% isopropylamine) 15% v/v; flowrate 2.5 mL/min; pressure 120 bar; temp. 38° C.; UV detection 220 nm; loop 25 L): RT 15.6 minutes

¹H NMR (400 MHZ, CD₃OD) δ 7.89-7.72 (m, 1H), 6.85-6.75 (m, 1H), 5.35 (dd, J=4.1, 11.3 Hz, 1H), 5.30-5.23 (m, 1H), 4.37 (d, J=11.3 Hz, 1H), 4.27-3.97 (m, 4H), 3.90-3.70 (m, 2H), 3.54-3.43 (m, 2H), 3.39 (d, J=11.7 Hz, 1H), 3.12-2.96 (m, 1H), 2.74-2.57 (m, 1H), 2.32 (s, 3H), 2.42-2.28 (m, 1H), 2.21 (br d, J=11.7 Hz, 1H), 2.08-1.25 (m, 8H), 1.24-1.13 (m, 1H).

### Ip-1 Accumulation Assay

The accumulation of Inositol-1 Monophosphate (IP-1) was measured using IP-One HTRF® Terbium cryptate based assay (Cisbio) in human recombinant OX1 (hOX1) and at OX2 (hOX2) receptors expressed in CHO cells (DiscoverX) according to the manufacturer's instructions for cells tested in suspension.

hOX1-CHO and hOX2-CHO cells were seeded into white 384-well plates at a density of 20,000 cells/well in Hank's Balanced Salt Solution (HBSS) containing 20 mM HEPES pH 7.4, 50 mM, LiCl and 0.1% and Bovine Serum Albumin (BSA).

Compounds of disclosure were tested in an 11 points concentration response curve (CRC) serially diluted in neat DMSO at 200 fold concentrations and added by Echo acoustic liquid handling (Labcyte) to the cells (0.5% DMSO final in the assay). After 60 min of incubation at 37° C. detection reagents, IP1-d2 tracer and anti-IP1-cryptate were diluted in lysis buffer according to the manufacturer's descriptions and added to the cells.

Following 60 min incubation at room temperature, time-resolved fluorescence (HTRF) was measured at 615 nm and 665 nm by Envision Multilabel reader (Perkin Elmer) and the HTRF ratio (A665/A615×10⁴) was calculated.

The IP-1 accumulation response was expressed as percentage of the maximal OX-A response.

Curve fitting and EC₅₀ estimations were carried out using a four-parameter logistic model using XLfit Software. Mean data of EC₅₀ are calculated from at least two independent experiments performed in duplicates

Category A corresponds to compounds displaying an IC50<100 nM, Category B between 100 nM and 1,000 nM, Category C between 1,000 nM and 10,000 nM and Category D above 10,000 nM

TABLE 2
Biological Evaluation of Disclosed Compounds

| Example | | | | EC50_OX2R |
|---|---|---|---|---|
| no. | Structure | Name | Comment | Category |
| 1 | 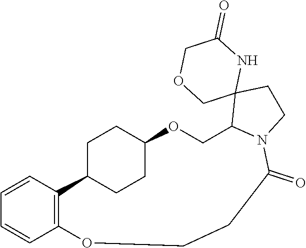 [SMILES: O=C1COCC2(CCN3C(=O)CCOc4ccccc4[C@H]4CC[C@H](CC4)OCC32)N1] | rel-(1′s,3S,16′R,19′s)-8′,18′- dioxa-12′- azaspiro[morpholine-3,15′- tetracyclo[17.2.2.0²,⁷.0¹²,¹⁶] tricosane]-2′(7′),3′,5′-triene- 5,11′-dione | Racemic- CIS | A |
| 1a | 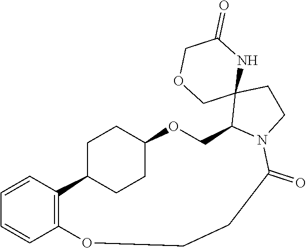 [SMILES: O=C1COC[C@@]2(CCN3C(=O)CCOc4ccccc4[C@H]4CC[C@H](CC4)OC[C@H]32)N1] | (1′s,3S,16′R,19′s)-8′,18′- dioxa-12′- azaspiro[morpholine-3,15′- tetracyclo[17.2.2.0²,⁷.0¹²,¹⁶] tricosane]-2′(7′),3′,5′-triene- 5,11′-dione | Isomer 1 | A |
| 1b | 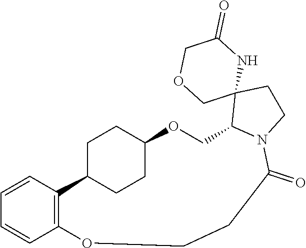 [SMILES: O=C1COC[C@]2(CCN3C(=O)CCOc4ccccc4[C@H]4CC[C@H](CC4)OC[C@@H]32)N1] | (1′s,3R,16′S,19′s)-8′,18′- dioxa-12′- azaspiro[morpholine-3,15′- tetracyclo[17.2.2.0²,⁷.0¹²,¹⁶] tricosane]-2′(7′),3′,5′-triene- 5,11′-dione | Isomer 2 | D |
| 2 | 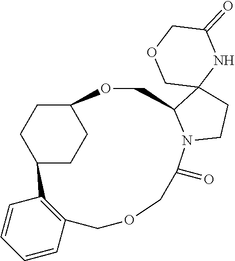 [SMILES: O=C1COCC2(CCN3C(=O)COCc4ccccc4[C@H]4CC[C@H](CC4)OC[C@H]32)N1] | rel-(1′s,3S,17′R,20′s)-8′,19′- dioxa-13′- azaspiro[morpholine-3,16′- tetracyclo[18.2.2.0²,⁷.0¹³,¹⁷] tetracosane]-2′(7′),3′,5′-triene- 5,12′-dione | Racemic- CIS | C |
| 3 | 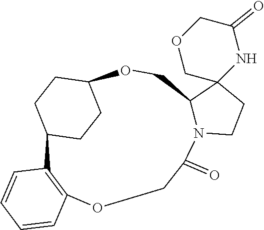 [SMILES: O=C1COCC2(CCN3C(=O)COc4ccccc4[C@H]4CC[C@H](CC4)OC[C@H]32)N1] | rel-(1′s,3S,15′R,18′s)-8′,17′- dioxa-11′- azaspiro[morpholine-3,14′- tetracyclo[16.2.2.0²,⁷.0¹¹,¹⁵] docosane]-2′(7′),3′,5′-triene- 5,10′-dione | Racemic- CIS | C |
| 4 | 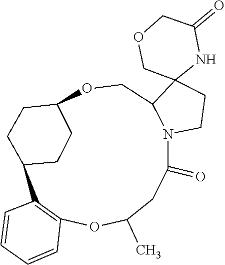 [SMILES: CC1CC(=O)N2CCC3(COCC(=O)N3)C2CO[C@H]2CC[C@H](CC2)c2ccccc2O1] | rel-(1′s,3S,16′R,19′s)-9′- methyl-8′,18′-dioxa-12′- azaspiro[morpholine-3,15′- tetracyclo[17.2.2.0²,⁷.0¹²,¹⁶] tricosane]-2′(7′),3′,5′-triene- 5,1l′-dione | Diastereo- isomeric mixture | B |
| 4a | 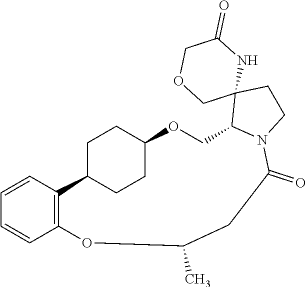 [SMILES: C[C@H]1CC(=O)N2CC[C@]3(COCC(=O)N3)[C@H]2CO[C@H]2CC[C@H](CC2)c2ccccc2O1] | (1′s,3R,9′S,16′S,19′s)-9′- methy1-8′,18′-dioxa-12′- azaspiro[morpholine-3,15′- tetracyclo[17.2.2.0²,⁷.0¹²,¹⁶] tricosane]-2′(7′),3′,5′-triene- 5,11′-dione | Isomer 1 | D |
| 4b | 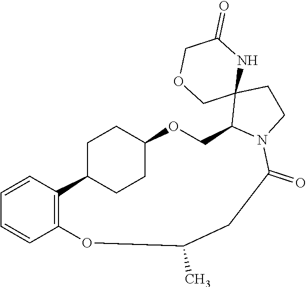 [SMILES: C[C@H]1CC(=O)N2CC[C@@]3(COCC(=O)N3)[C@@H]2CO[C@H]2CC[C@H](CC2)c2ccccc2O1] | (1′s,3S,9′S,16′R,19′s)-9′- methy1-8′,18′-dioxa-12′- azaspiro[morpholine-3,15′- tetracyclo[17.2.2.0²,⁷.0¹²,¹⁶] tricosane]-2′(7′),3′,5′-triene- 5,11′-dione | Isomer 2 | B |
| 4c | 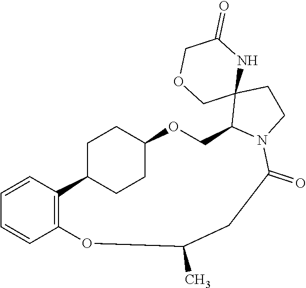 [SMILES: C[C@@H]1CC(=O)N2CC[C@@]3(COCC(=O)N3)[C@@H]2CO[C@H]2CC[C@H](CC2)c2ccccc2O1] | (1′s,3S,9′R,16′R,19′s)-9′- methy1-8′,18′-dioxa-12′- azaspiro[morpholine-3,15′- tetracyclo[17.2.2.0²,⁷.0¹²,¹⁶] tricosane]-2′(7′),3′,5′-triene- 5,11′-dione | Isomer 3 | B |
| 4d | 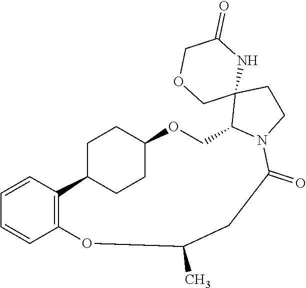 [SMILES: C[C@@H]1CC(=O)N2CC[C@]3(COCC(=O)N3)[C@H]2CO[C@H]2CC[C@H](CC2)c2ccccc2O1] | (1′s,3R,9′R,16′S,19′s)-9′- methy1-8′,18′-dioxa-12′- azaspiro[morpholine-3,15′- tetracyclo[17.2.2.0²,⁷.0¹²,¹⁶] tricosane]-2′(7′),3′,5′-triene- 5,11′-dione | Isomer 4 | D |
| 5 | 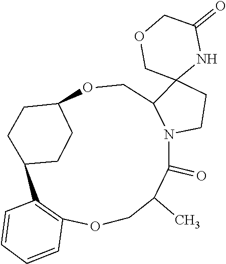 [SMILES: CC1COc2ccccc2[C@H]2CC[C@H](CC2)OCC2N(CCC23COCC(=O)N3)C1=O] | rel-(1′s,3S,16′R,19′s)-10′- methy1-8′,18′-dioxa-12′- azaspiro[morpholine-3,15′- tetracyclo[17.2.2.0²,⁷.0¹²,¹⁶] tricosane]-2′(7′),3′,5′-triene- 5,11′-dione | Diastereo- isomeric mixture | B |
| 5a | 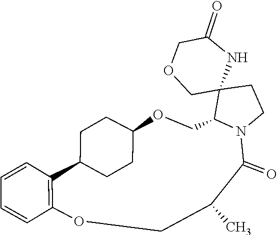 [SMILES: C[C@@H]1COc2ccccc2[C@H]2CC[C@H](CC2)OC[C@H]2N(CC[C@]23COCC(=O)N3)C1=O] | (1′s,3R,10′R,16′S,19′s)-10′- methyl-8′,18′-dioxa-12′- azaspiro[morpholine-3,15′- tetracyclo[17.2.2.0²,⁷.0¹²,¹⁶] tricosane]-2′(7′),3′,5′-triene- 5,11′-dione | Isomer 1 | D |
| 5b | 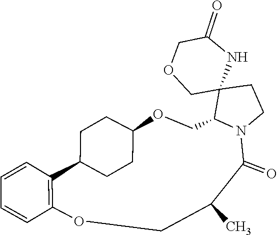 [SMILES: C[C@H]1COc2ccccc2[C@H]2CC[C@H](CC2)OC[C@H]2N(CC[C@]23COCC(=O)N3)C1=O] | (1′s,3R,10′S,16′S,19′s)-10′- methy1-8′,18′-dioxa-12′- azaspiro[morpholine-3,15′- tetracyclo[17.2.2.0²,⁷.0¹²,¹⁶] tricosane]-2′(7′),3′,5′-triene- 5,11′-dione | Isomer B | D |
| 5c | 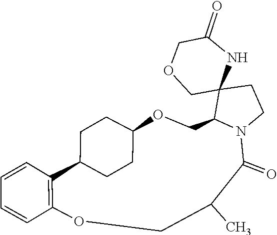 [SMILES: CC1COc2ccccc2[C@H]2CC[C@H](CC2)OC[C@@H]2N(CC[C@@]23COCC(=O)N3)C1=O] | rel-(1′s,3S,16′R,19′s)-10′- methyl-8′,18′-dioxa-12′- azaspiro[morpholine-3,15′- tetracyclo[17.2.2.0²,⁷.0¹²,¹⁶] tricosane]-2′(7′),3′,5′-triene- 5,11′-dione | Racemic | B |
| 6 | 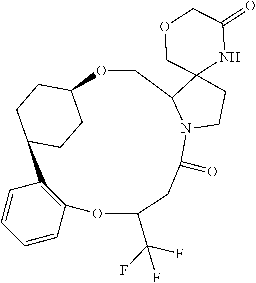 [SMILES: O=C1COCC2(CCN3C(=O)CC(C(F)(F)F)Oc4ccccc4[C@H]4CC[C@H](CC4)OCC32)N1] | rel-(1′s,3S,16′R,19′s)-9′- (trifluoromethyl)-8′,18′- dioxa-12′- azaspiro[morpholine-3,15′- tetracyclo[17.2.2.0²,⁷.0¹²,¹⁶] tricosane]-2′(7′),3′,5′-triene- 5,11′-dione | Racemic | C |
| 7 | 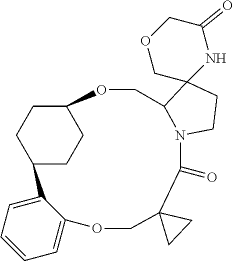 [SMILES: O=C1COCC2(CCN3C(=O)C4(CC4)COc4ccccc4[C@H]4CC[C@H](CC4)OCC32)N1] | rel-(1′s,15′S,16′R,19′s)- dispiro[cyclopropane-1,10′- [8,18]dioxa-[12] azatetracyclo[17.2.2.0²,⁷.0¹²,¹⁶] tricosane-15′,3″″- morpholine]-2′(7′),3′,5′- triene-5″,11′-dione | Racemic | C |
| 8 | 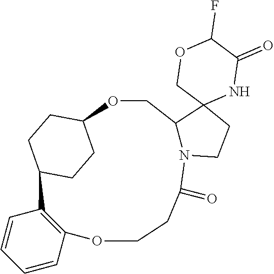 [SMILES: O=C1NC2(CCN3C(=O)CCOc4ccccc4[C@H]4CC[C@H](CC4)OCC32)COC1F] | rel-(1′s,3S,16′R,19′s)-6- fluoro-8′,18′-dioxa-12′- azaspiro[morpholine-3,15′- tetracyclo[17.2.2.0²,⁷.0¹²,¹⁶] tricosane]- 2′(7′),3′,5′-triene-5,1l′-dione | Racemic | A |
| 9 | 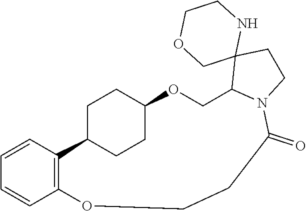 [SMILES: O=C1CCOc2ccccc2[C@H]2CC[C@H](CC2)OCC2N1CCC21COCCN1] | Rel-(1′s,3S,16′R,19′s)-8′,18′- dioxa-12′- azaspiro[morpholine-3,15′- tetracyclo[17.2.2.0²,⁷.0¹²,¹⁶] tricosane]-2′,4′,6′-trien-11′-one | Racemic | D |
| 10 | 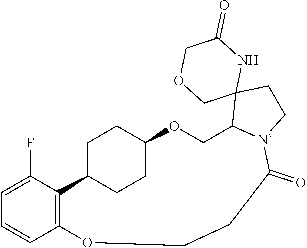 | Rel-(1′s,3S,6′R,19′s)-3′- fluoro-8′,18′-dioxa-12′- azaspiro[morpholine-3,15′- tetracyclo[17.2.2.0²,⁷.0¹²,¹⁶] tricosane]-2′(7′),3′,5′-triene- 5,11′-dione | Racemic | A |
| 10a | 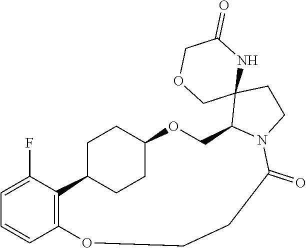 [SMILES: O=C1COC[C@@]2(CCN3C(=O)CCOc4cccc(F)c4[C@H]4CC[C@H](CC4)OC[C@H]32)N1] | (1′s,3S,16′R,19′s)-3′-fluoro- 8′,18′-dioxa-12′- azaspiro[morpholine-3,15′- tetracyclo[17.2.2.0²,⁷.0¹²,¹⁶] tricosane]-2′(7′),3′,5′-triene- 5,11′-dione | Isomer 1 | A |
| 10b | 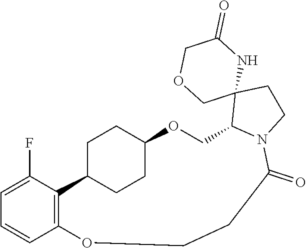 [SMILES: O=C1COC[C@]2(CCN3C(=O)CCOc4cccc(F)c4[C@H]4CC[C@H](CC4)OC[C@@H]32)N1] | (1′s,3R,16′S,19′s)-3′-fluoro- 8′,18′-dioxa-12′- azaspiro[morpholine-3,15′- tetracyclo[17.2.2.0²,⁷.0¹²,¹⁶] tricosane]-2′(7′),3′,5′-triene- 5,11′-dione | Isomer 2 | D |
| 11 | 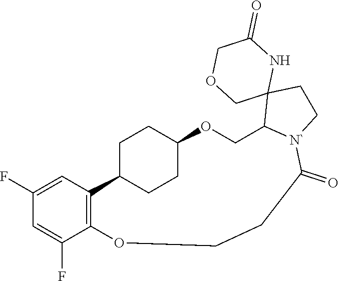 | Rel-(1′s,3S,16′R,19′s)-4′,6′- difluoro-8′,18′-dioxa-12′- azaspiro[morpholine-3,15′- tetracyclo[17.2.2.0^{2,7}.0^{12,16}] tricosane]-2′(7′),3′,5′-triene- 5,11′-dione | Racemic- | B |
| 12a | 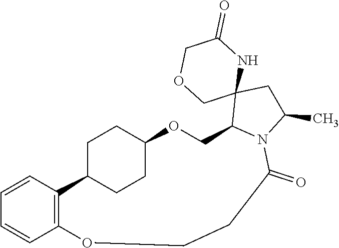 [SMILES: C[C@@H]1C[C@@]2(COCC(=O)N2)[C@@H]2CO[C@H]3CC[C@H](CC3)c3ccccc3OCCC(=O)N12] | (1′s,3S,12′R,15′R,18′s)-12′- methyl-8′,17′-dioxa-11′- azaspiro[morpholine-3,14′- tetracyclo[16.2.2.0²,⁷.0¹¹,¹⁵] docosane]-2′(7′),3′,5′-triene- 5,10′-dione | Isomer 1 | A |
| 12b | 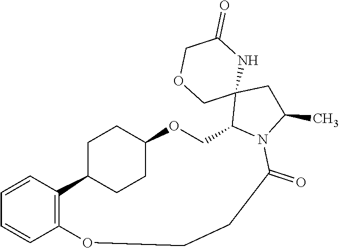 [SMILES: C[C@@H]1C[C@]2(COCC(=O)N2)[C@H]2CO[C@H]3CC[C@H](CC3)c3ccccc3OCCC(=O)N12] | (1′s,3R,12′R,15′S,18′s)-12′- methy1-8′,17′-dioxa-11′- azaspiro[morpholine-3,14′- tetracyclo[16.2.2.0²,⁷.0¹¹,¹⁵]do cosane]-2′(7′),3′,5′-triene- 5,10′-dione | Isomer 2 | B |
| 13 | 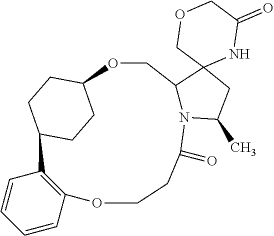 [SMILES: C[C@@H]1CC2(COCC(=O)N2)C2CO[C@H]3CC[C@H](CC3)c3ccccc3OCCC(=O)N21] | rel-(1′s,3S,13′R,16′R,19′s)- 13′-methyl-8′,18′-dioxa-12′- azaspiro[morpholine-3,15′- tetracyclo[17.2.2.02,7.012,16] tricosane]-2′(7′),3′,5′-triene- 5,11′-dione | Diastereo- isomeric mixture | A |
| 13a | 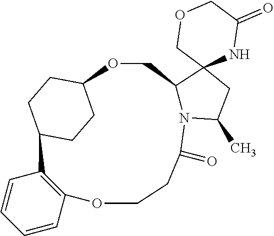 [SMILES: C[C@@H]1C[C@@]2(COCC(=O)N2)[C@@H]2CO[C@H]3CC[C@H](CC3)c3ccccc3OCCC(=O)N12] | (1′s,3S,13′R,16′R,19′s)-13′- methy1-8′,18′-dioxa-12′- azaspiro[morpholine-3,15′- tetracyclo[17.2.2.0²,⁷.0¹²,¹⁶] tricosane]-2′(7′),3′,5′-triene- 5,11′-dione | Isomer A | A |
| 13b | 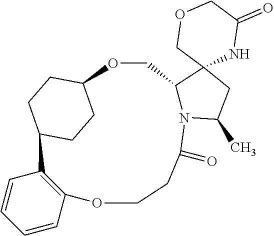 [SMILES: C[C@@H]1C[C@]2(COCC(=O)N2)[C@H]2CO[C@H]3CC[C@H](CC3)c3ccccc3OCCC(=O)N12] | (1′s,3R,13′R,16′S,19′s)-13′- methy1-8′,18′-dioxa-12′- azaspiro[morpholine-3,15′- tetracyclo[17.2.2.0²,⁷.0¹²,¹⁶] tricosane]-2′(7′),3′,5′-triene- 5,11′-dione | Isomer B | B |
| 14 | 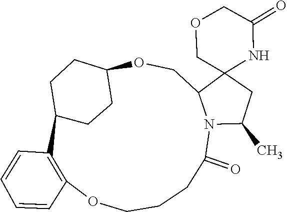 [SMILES: C[C@@H]1CC2(COCC(=O)N2)C2CO[C@H]3CC[C@H](CC3)c3ccccc3OCCCC(=O)N21] | (1′s,14′R,20′s)-14′-methyl- 8′,19′-dioxa-13′- azaspiro[morpholine-3,16′- tetracyclo[18.2.2.0²,⁷.0¹³,¹⁷] tetracosane]-2′(7′),3′,5′-triene- 5,12′-dione | Racemic | A |
| 15a | 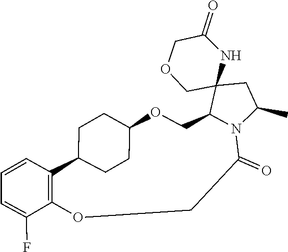 [SMILES: C[C@@H]1C[C@@]2(COCC(=O)N2)[C@@H]2CO[C@H]3CC[C@H](CC3)c3cccc(F)c3OCC(=O)N12] | (1′s,3S,12′R,15′R,18′s)-6′- fluoro-12′-methyl-8′,17′- dioxa-11′- azaspiro[morpholine-3,14′- tetracyclo[16.2.2.0²,⁷.0¹¹,¹⁵] docosane]-2′,4′,6′-triene-5,10′- dione | Isomer 1 | A |
| 15b | 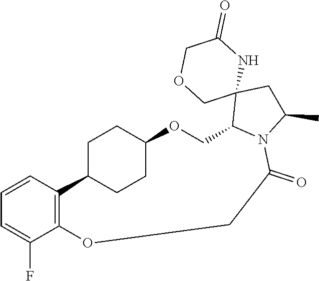 [SMILES: C[C@@H]1C[C@]2(COCC(=O)N2)[C@H]2CO[C@H]3CC[C@H](CC3)c3cccc(F)c3OCC(=O)N12] | (1′s,3R,12′R,15′S,18′s)-6′- fluoro-12′-methyl-8′,17′- dioxa-11′- azaspiro[morpholine-3,14′- tetracyclo[16.2.2.0²,⁷.0¹¹,¹⁵] docosane]-2′,4′,6′-triene-5,10′- dione | Isomer 2 | C |
| 16a | 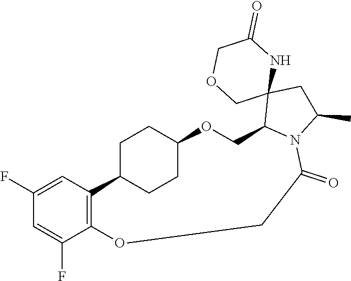 [SMILES: C[C@@H]1C[C@@]2(COCC(=O)N2)[C@@H]2CO[C@H]3CC[C@H](CC3)c3cc(F)cc(F)c3OCC(=O)N12] | (1′s,3S,12′R,15′R,18′s)-4′,6′- difluoro-12′-methyl-8′,17′- dioxa-11′- azaspiro[morpholine-3,14′- tetracyclo[16.2.2.0²,⁷.0¹¹,¹⁵] docosane]-2′,4′,6′-triene-5,10′- dione | Isomer 1 | A |
| 16b | 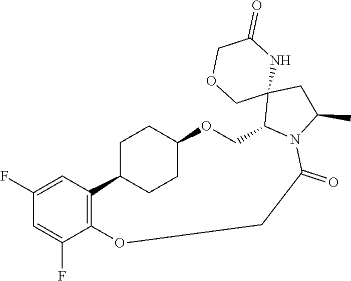 [SMILES: C[C@@H]1C[C@]2(COCC(=O)N2)[C@H]2CO[C@H]3CC[C@H](CC3)c3cc(F)cc(F)c3OCC(=O)N12] | (1′s,3R,12′R,15′S,18′s)-4′,6′- difluoro-12′-methy1-8′,17′- dioxa-11′- azaspiro[morpholine-3,14′- tetracyclo[16.2.2.0²,⁷.0¹¹,¹⁵] docosane]-2′,4′,6′-triene-5,10′- dione | Isomer 2 | D |
| 17a | 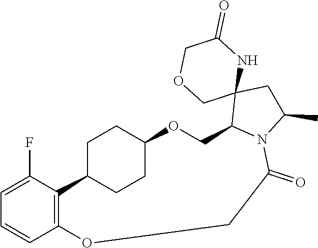 [SMILES: C[C@@H]1C[C@@]2(COCC(=O)N2)[C@@H]2CO[C@H]3CC[C@H](CC3)c3c(F)cccc3OCC(=O)N12] | (1′s,3S,12′R,15′R,18′s)-3′- fluoro-12′-methyl-8′,17′- dioxa-11′- azaspiro[morpholine-3,14′- tetracyclo[16.2.2.0²,⁷.0¹¹,¹⁵] docosane]-2′,4′,6′-triene-5,10′- dione | Isomer 1 | A |
| 17b | 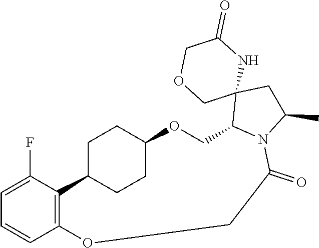 [SMILES: C[C@@H]1C[C@]2(COCC(=O)N2)[C@H]2CO[C@H]3CC[C@H](CC3)c3c(F)cccc3OCC(=O)N12] | (1′s,3R,12′R,15′S,18′s)-3′- fluoro-12′-methyl-8′,17′- dioxa-11′- azaspiro[morpholine-3,14′- tetracyclo[16.2.2.0²,⁷.0¹¹,¹⁵] docosane]-2′,4′,6′-triene-5,10′- dione | Isomer 2 | C |
| 18a | 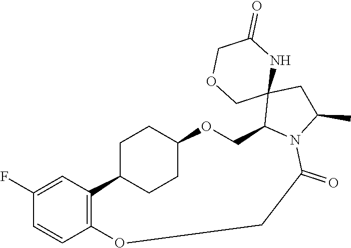 [SMILES: C[C@@H]1C[C@@]2(COCC(=O)N2)[C@@H]2CO[C@H]3CC[C@H](CC3)c3cc(F)ccc3OCC(=O)N12] | (1′s,3S,12′R,15′R,18′s)-4′- fluoro-12′-methyl-8′,17′- dioxa-11′- azaspiro[morpholine-3,14′- tetracyclo[16.2.2.0²,⁷.0¹¹,¹⁵] docosane]-2′,4′,6′-triene-5,10′- dione | Isomer 1 | A |
| 18b | 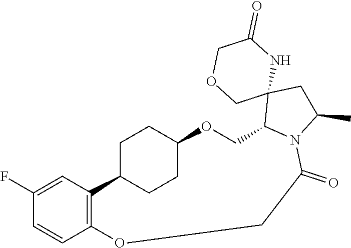 [SMILES: C[C@@H]1C[C@]2(COCC(=O)N2)[C@H]2CO[C@H]3CC[C@H](CC3)c3cc(F)ccc3OCC(=O)N12] | (1′s,3R,12′R,15′S,18′s)-4′- fluoro-12′-methyl-8′,17′- dioxa-11′- azaspiro[morpholine-3,14′- tetracyclo[16.2.2.0²,⁷.0¹¹,¹⁵] docosane]-2′,4′,6′-triene-5,10′- dione | Isomer 2 | D |
| 19a | 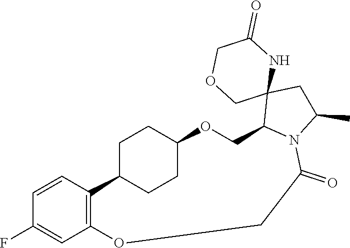 [SMILES: C[C@@H]1C[C@@]2(COCC(=O)N2)[C@@H]2CO[C@H]3CC[C@H](CC3)c3ccc(F)cc3OCC(=O)N12] | (1′s,3S,12′R,15′R,18′s)-5′- fluoro-12′-methyl-8′,17′- dioxa-11′- azaspiro[morpholine-3,14′- tetracyclo[16.2.2.0²,⁷.0¹¹,¹⁵] docosane]-2′,4′,6′-triene-5,10′- dione | Isomer 1 | A |
| 19b | 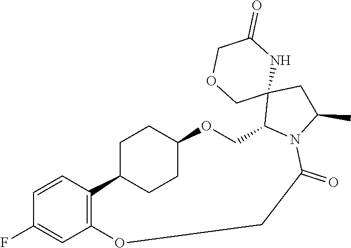 [SMILES: C[C@@H]1C[C@]2(COCC(=O)N2)[C@H]2CO[C@H]3CC[C@H](CC3)c3ccc(F)cc3OCC(=O)N12] | (1′s,3R,12′R,15′S,18′s)-5′- fluoro-12′-methyl-8′,17′- dioxa-11′- azaspiro[morpholine-3,14′- tetracyclo[16.2.2.0²,⁷.0¹¹,¹⁵] docosane]-2′,4′,6′-triene-5,10′- dione | Isomer 2 | D |
| 20a | 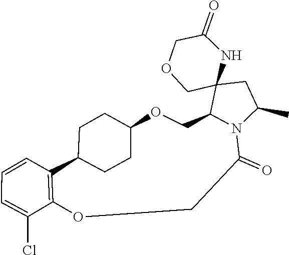 [SMILES: C[C@@H]1C[C@@]2(COCC(=O)N2)[C@@H]2CO[C@H]3CC[C@H](CC3)c3cccc(Cl)c3OCC(=O)N12] | (1′s,3S,12′R,15′R,18′s)-6′- chloro-12′-methy1-8′,17′- dioxa-11′- azaspiro[morpholine-3,14′- tetracyclo[16.2.2.0²,⁷.0¹¹,¹⁵] docosane]-2′,4′,6′-triene-5,10′- dione | Isomer 1 | C |
| 20b | 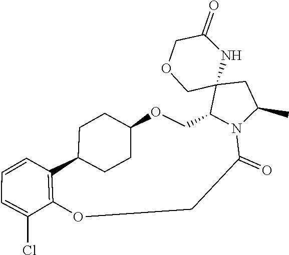 [SMILES: C[C@@H]1C[C@]2(COCC(=O)N2)[C@H]2CO[C@H]3CC[C@H](CC3)c3cccc(Cl)c3OCC(=O)N12] | (1′s,3R,12′R,15′S,18′s)-6′- chloro-12′-methyl-8′,17′- dioxa-11′- azaspiro[morpholine-3,14′- tetracyclo[16.2.2.0²,⁷.0¹¹,¹⁵] docosane]-2′(7′),3′,5′-triene- 5,10′-dione | Isomer 2 | D |
| 21a | 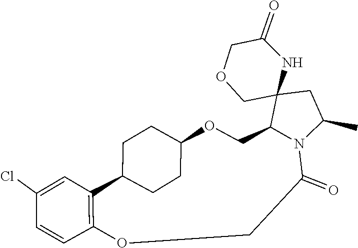 [SMILES: C[C@@H]1C[C@@]2(COCC(=O)N2)[C@@H]2CO[C@H]3CC[C@H](CC3)c3cc(Cl)ccc3OCC(=O)N12] | (1′s,3S,12′R,15′R,18′s)-4′- chloro-12′-methyl-8′,17′- dioxa-11′- azaspiro[morpholine-3,14′- tetracyclo[16.2.2.0²,⁷.0¹¹,¹⁵] docosane]-2′,4′,6′-triene-5,10′- dione | Isomer 1 | B |
| 21b | 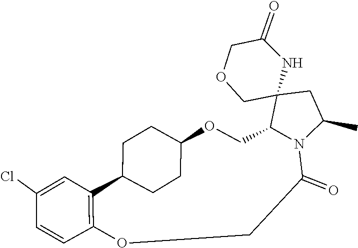 [SMILES: C[C@@H]1C[C@]2(COCC(=O)N2)[C@H]2CO[C@H]3CC[C@H](CC3)c3cc(Cl)ccc3OCC(=O)N12] | (1′s,3R,12′R,15′S,18′s)-4′- chloro-12′-methyl-8′,17′- dioxa-11′- azaspiro[morpholine-3,14′- tetracyclo[16.2.2.0²,⁷.0¹¹,¹⁵] docosane]-2′(7′),3′,5′-triene- 5,10′-dione | Isomer 2 | D |
| 22a | 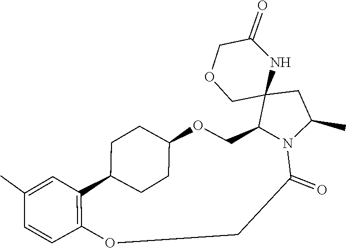 | (1′s,3S,12′R,15′R,18′s)-4′,12′- dimethy1-8′,17′-dioxa-11′- azaspiro[morpholine-3,14′- tetracyclo[16.2.2.0²,⁷.0¹¹,¹⁵] docosane]-2′,4′,6′-triene-5,10′- dione | Isomer 1 | A |
| 22b | 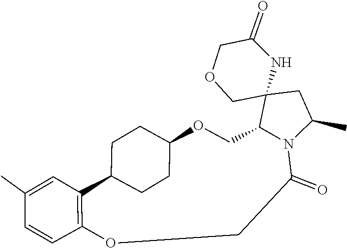 | (1′s,3R,12′R,15′S,18′s)-4′,12′- dimethy1-8′,17′-dioxa-11′- azaspiro[morpholine-3,14′- tetracyclo[16.2.2.0²,⁷.0¹¹,¹⁵] docosane]-2′,4′,6′-triene-5,10′- dione | Isomer 2 | D |
| 23a | 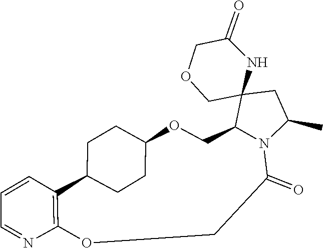 [SMILES: C[C@@H]1C[C@@]2(COCC(=O)N2)[C@@H]2CO[C@H]3CC[C@H](CC3)c3cccnc3OCC(=O)N12] | (1′s,3S,12′R,15′R,18′s)-12′- methy1-8′,17′-dioxa-6′,11′- diazaspiro[morpholine-3,14′- tetracyclo[16.2.2.0²,⁷.0¹¹,¹⁵] docosane]-2′,4′,6′-triene-5,10′- dione | Isomer 1 | B |
| 23b | 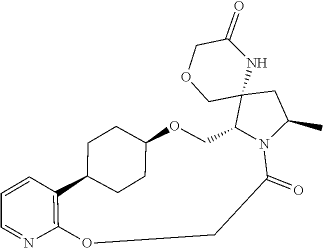 [SMILES: C[C@@H]1C[C@]2(COCC(=O)N2)[C@H]2CO[C@H]3CC[C@H](CC3)c3cccnc3OCC(=O)N12] | (1′s,3R,12′R,15′S,18′s)-12′- methyl-8′,17′-dioxa-6′,11′- diazaspiro[morpholine-3,14′- tetracyclo[16.2.2.0²,⁷.0¹¹,¹⁵] docosane]-2′,4′,6′-triene-5,10′- dione | Isomer 2 | C |
| 24a | 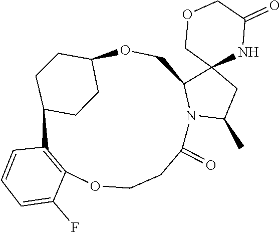 [SMILES: C[C@@H]1C[C@@]2(COCC(=O)N2)[C@@H]2CO[C@H]3CC[C@H](CC3)c3cccc(F)c3OCCC(=O)N12] | (1′s,3S,13′R,16′R,19′s)-6′- fluoro-13′-methyl-8′,18′- dioxa-12′- azaspiro[morpholine-3,15′- tetracyclo[17.2.2.0²,⁷.0¹²,¹⁶] tricosane]-2′(7′),3′,5′-triene- 5,11′-dione | Isomer 1 | A |
| 24b | 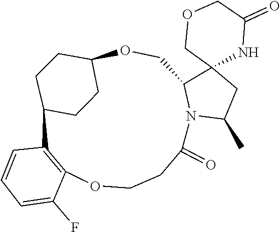 [SMILES: C[C@@H]1C[C@]2(COCC(=O)N2)[C@H]2CO[C@H]3CC[C@H](CC3)c3cccc(F)c3OCCC(=O)N12] | (1′s,3R,13′R,16′S,19′s)-6′- fluoro-13′-methyl-8′,18′- dioxa-12′- azaspiro[morpholine-3,15′- tetracyclo[17.2.2.0²,⁷.0¹²,¹⁶] tricosane]-2′(7′),3′,5′-triene- 5,1l′-dione | Isomer 2 | B |
| 25a | 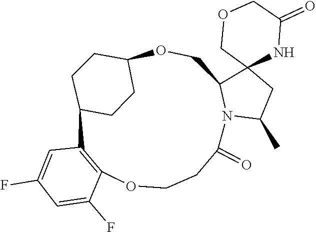 [SMILES: C[C@@H]1C[C@@]2(COCC(=O)N2)[C@@H]2CO[C@H]3CC[C@H](CC3)c3cc(F)cc(F)c3OCCC(=O)N12] | (1′s,3S,13′R,16′R,19′s)-4′,6′- difluoro-13′-methyl-8′,18′- dioxa-12′- azaspiro[morpholine-3,15′- tetracyclo[17.2.2.0²,⁷.0¹²,¹⁶] tricosane]-2′,4′,6′-triene-5,11′- dione | Isomer 1 | A |
| 25b | 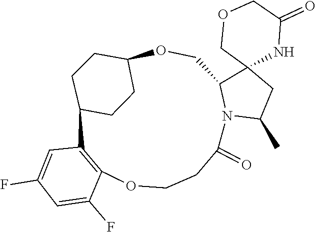 [SMILES: C[C@@H]1C[C@]2(COCC(=O)N2)[C@H]2CO[C@H]3CC[C@H](CC3)c3cc(F)cc(F)c3OCCC(=O)N12] | (1′s,3R,13′R,16′S,19′s)-4′,6′- difluoro-13′-methyl-8′,18′- dioxa-12′- azaspiro[morpholine-3,15′- tetracyclo[17.2.2.0²,⁷.0¹²,¹⁶]tri cosane]-2′,4′,6′-triene-5,11′- dione | Isomer 2 | A |
| 26 | 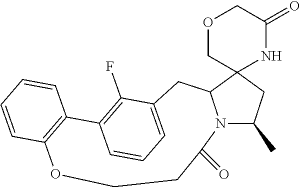 [SMILES: C[C@@H]1CC2(COCC(=O)N2)C2Cc3cccc(c3F)-c3ccccc3OCCC(=O)N21] | (13′R)-22′-fluoro-13′-methyl- 8′-oxa-12′- azaspiro[morpholine-3,15′- tetracyclo[16.3.1.0^{2,7}.0¹²,¹⁶] docosane]- 1′(22′),2′(7′),3′,5′,18′,20′- hexaene-5,1l′-dione | Racemic- CIS | C |
| 27 | 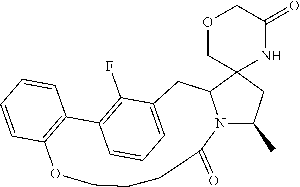 [SMILES: C[C@@H]1CC2(COCC(=O)N2)C2Cc3cccc(c3F)-c3ccccc3OCCCC(=O)N21] | (14′R)-23′-fluoro-14′-methyl- 8′-oxa-13′- azaspiro[morpholine-3,16′- tetracyclo[17.3.1.0^{2,7}.0¹³,¹⁷] tricosane]- 1′(23′),2′(7′),3′,5′,19′,21′- hexaene-5,12′-dione | Racemic- CIS | B |
| 28 | 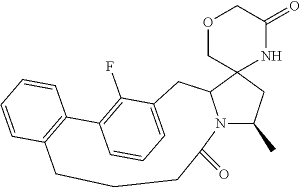 [SMILES: C[C@@H]1CC2(COCC(=O)N2)C2Cc3cccc(c3F)-c3ccccc3CCCC(=O)N21] | (13′R)-22′-fluoro-13′-methyl- 12′-azaspiro[morpholine- 3,15′- tetracyclo[16.2.2.0²,⁷.0¹²,¹⁶] docosane]- 1′(22′),2′(7′),3′,5′,18′,20′- hexaene-5,11′-dione | Racemic- CIS | C |
| 29 | 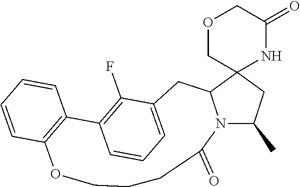 [SMILES: C[C@@H]1CC2(COCC(=O)N2)C2Cc3cccc(c3F)-c3ccccc3OCCCC(=O)N21] | (14′R)-23′-fluoro-14′-methyl- 13′-azaspiro[morpholine- 3,16′- tetracyclo[17.3.1.0^{2,7}.0¹³,¹⁷] tricosane]- 1′(23′),2′(7′),3′,5′,19′,21′- hexaene-5,12′-dione | Racemic- CIS | D |
| 30 | 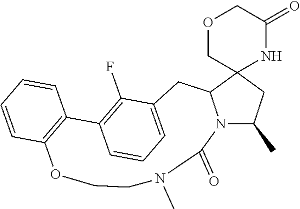 [SMILES: C[C@@H]1CC2(COCC(=O)N2)C2Cc3cccc(c3F)-c3ccccc3OCCN(C)C(=O)N21] | (14′R)-23′-fluoro-11′,14′- dimethyl-8′-oxa-11′,13′- diazaspiro[morpholine-3,16′- tetracyclo[17.3.1.0^{2,7}.0¹³,¹⁷] tricosane]- 1′(23′),2′(7′),3′,5′,19′,21′- hexaene-5,12′-dione | Racemic- CIS | C |
| 31 | 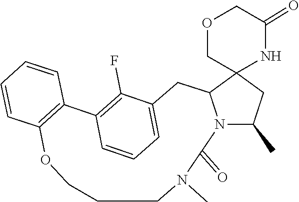 [SMILES: C[C@@H]1CC2(COCC(=O)N2)C2Cc3cccc(c3F)-c3ccccc3OCCCN(C)C(=O)N21] | (15′R)-24′-fluoro-12′,15′- dimethyl-8′-oxa-12′,14′- diazaspiro[morpholine-3,17′- tetracyclo[18.3.1.0^{2,7}.0¹⁴,¹⁸] tetracosane]- 1′(24′),2′(7′),3′,5′,20′,22′- hexaene-5,13′-dione | Racemic- CIS | D |
| 32 | 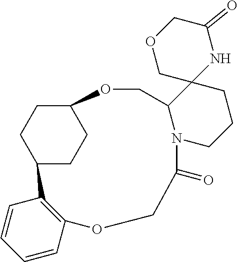 [SMILES: O=C1COCC2(CCCN3C(=O)COc4ccccc4[C@H]4CC[C@H](CC4)OCC32)N1] | Rel-(1′s,3S,16′R,19′s)-8′,18′- dioxa-11′- azaspiro[morpholine-3,15′- tetracyclo[17.3.1.0²,⁷.0¹¹,¹⁶] tricosane]-2′(7′),3′,5′-triene- 5,10′-dione | Racemic- CIS | A |
| 32a | 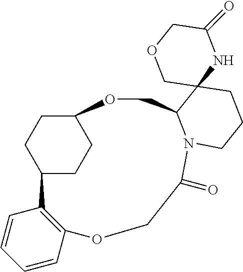 [SMILES: O=C1COC[C@@]2(CCCN3C(=O)COc4ccccc4[C@H]4CC[C@H](CC4)OC[C@H]32)N1] | (1′s,3S,16′R,19′s)-8′,18′- dioxa-11′- azaspiro[morpholine-3,15′- tetracyclo[17.3.1.0²,⁷.0¹¹,¹⁶] tricosane]-2′(7′),3′,5′-triene- 5,10′-dione | Isomer 1 | A |
| 32b | 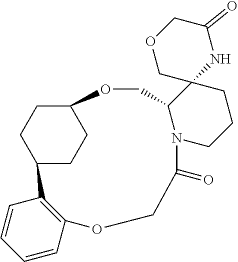 [SMILES: O=C1COC[C@]2(CCCN3C(=O)COc4ccccc4[C@H]4CC[C@H](CC4)OC[C@@H]32)N1] | (1′s,3R,16′S,19′s)-8′,18′- dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 2 | D |
| 33 | 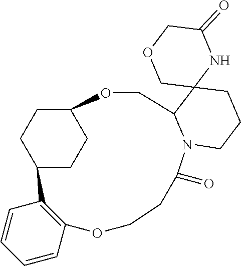 [SMILES: O=C1COCC2(CCCN3C(=O)CCOc4ccccc4[C@H]4CC[C@H](CC4)OCC32)N1] | Rel-(1′s,3S,17′R,20′s)-8′,19′- dioxa-12′- azaspiro[morpholine-3,16′- tetracyclo[18.2.2.0²,⁷.0¹²,¹⁷] tetracosane]-2′(7′),3′,5′-triene- 5,11′-dione | Racemic- CIS | A |
| 33a | 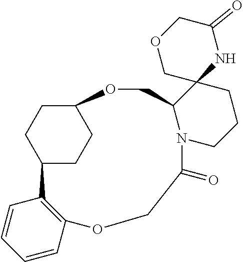 [SMILES: O=C1COC[C@@]2(CCCN3C(=O)COc4ccccc4[C@H]4CC[C@H](CC4)OC[C@H]32)N1] | (1′s,3S,17′R,20′s)-8′,19′- dioxa-12′- azaspiro[morpholine-3,16′- tetracyclo[18.2.2.0²,⁷.0¹²,¹⁷] tetracosane]-2′(7′),3′,5′-triene- 5,1l′-dione | Isomer 1 | A |
| 33b | 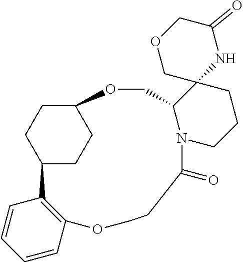 [SMILES: O=C1COC[C@]2(CCCN3C(=O)CCOc4ccccc4[C@H]4CC[C@H](CC4)OC[C@@H]32)N1] | (1′s,3R,17′S,20′s)-8′,19′- dioxa-12′- azaspiro[morpholine-3,16′- tetracyclo[18.2.2.0²,⁷.0¹²,¹⁷] tetracosane]-2′(7′),3′,5′-triene- 5,11′-dione | Isomer 2 | B |
| 34 | 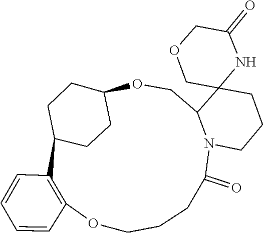 [SMILES: O=C1COCC2(CCCN3C(=O)CCCOc4ccccc4[C@H]4CC[C@H](CC4)OCC32)N1] | Rel-(1′s,3S,18′R,21′s)-8′,20′- dioxa-13′- azaspiro[morpholine-3,17′- tetracyclo[19.2.2.0^{2,7}.0^{13,18}] pentacosane]- 2′(7′),3′,5′-triene-5,12′-dione | Racemic- CIS | A |
| 35 | 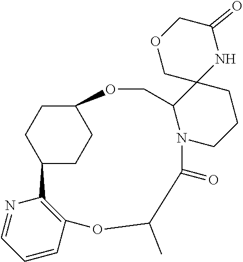 [SMILES: CC1Oc2cccnc2[C@H]2CC[C@H](CC2)OCC2N(CCCC23COCC(=O)N3)C1=O] | Rel-(1′s,3S,16′R,19′s)-9′- methy1-8′,18′-dioxa-11′- azaspiro[morpholine-3,15′- tetracyclo[17.2.2.02,7.011,16] tricosane]-2′(7′),3′,5′-triene- 5,10′-dione | Racemic- Cis | A |
| 35a | 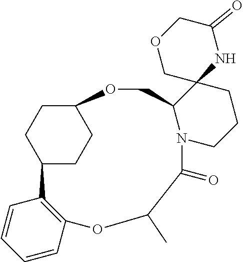 [SMILES: CC1Oc2ccccc2[C@H]2CC[C@H](CC2)OC[C@@H]2N(CCC[C@@]23COCC(=O)N3)C1=O] | (1′s,3S,16′R,19′s)-9′-methyl- 8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 1 | A |
| 35b | 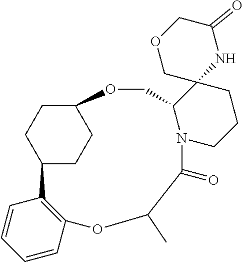 [SMILES: CC1Oc2ccccc2[C@H]2CC[C@H](CC2)OC[C@H]2N(CCC[C@]23COCC(=O)N3)C1=O] | (1′s,3R,16′S,19′s)-9′-methyl- 8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 2 | D |
| 36 | 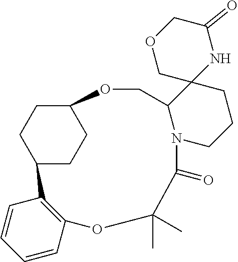 [SMILES: CC1(C)Oc2ccccc2[C@H]2CC[C@H](CC2)OCC2N(CCCC23COCC(=O)N3)C1=O] | Rel-(1′s,3S,16′R,19′s)-9′,9′- dimethyl-8′,18′-dioxa-11′- azaspiro[morpholine-3,15′- tetracyclo[17.2.2.02,7.011,16] tricosane]-2′(7′),3′,5′-triene- 5,10′-dione | Racemic- CIS | B |
| 37 | 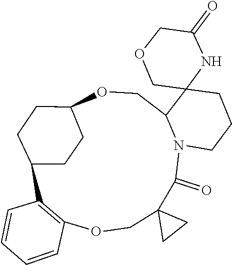 [SMILES: O=C1COCC2(CCCN3C(=O)C4(CC4)COc4ccccc4[C@H]4CC[C@H](CC4)OCC32)N1] | Rel-(1′s,16′S,17′R,20′s)- dispiro[cyclopropane-1,10′- [8,19]dioxa- [12]azatetracyclo [18.2.2.02,7.012,17] tetracosane-16′,3″- morpholine]-2′(7′),3′,5′- triene-5″,11′-dione | Racemic- CIS | A |
| 37a | 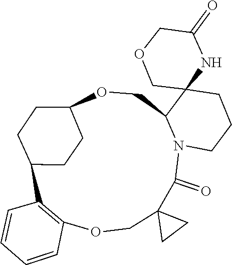 [SMILES: O=C1COC[C@@]2(CCCN3C(=O)C4(CC4)COc4ccccc4[C@H]4CC[C@H](CC4)OC[C@H]32)N1] | (1′s,16′S,17′R,20′s)- dispiro[cyclopropane-1,10′- [8,19]dioxa- [12]azatetracyclo[18.2.2.0²,⁷. 0¹²,¹⁷]tetracosane-16′,3″- morpholine]-2′(7′),3′,5′- triene-5″,11′-dione | Isomer 1 | A |
| 37b | 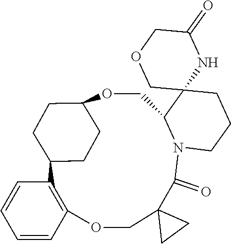 [SMILES: O=C1COC[C@]2(CCCN3C(=O)C4(CC4)COc4ccccc4[C@H]4CC[C@H](CC4)OC[C@@H]32)N1] | (1′s,16′R,17′S,20′s)- dispiro[cyclopropane-1,10′- [8,19]dioxa- [12]azatetracyclo[18.2.2.0²,⁷. 0¹²,¹⁷]tetracosane-16′,3″- morpholine]-2′(7′),3′,5′- triene-5″,11′-dione | Isomer 2 | C |
| 38a | 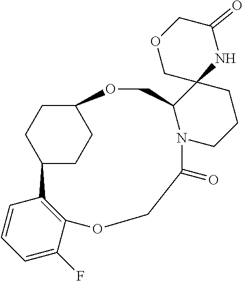 [SMILES: O=C1COC[C@@]2(CCCN3C(=O)COc4c(F)cccc4[C@H]4CC[C@H](CC4)OC[C@H]32)N1] | (1′s,3S,16′R,19′s)-6′-fluoro- 8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 1 | A |
| 38b | 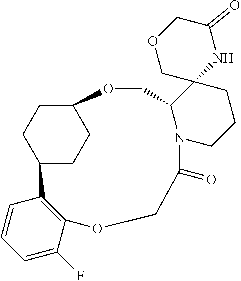 [SMILES: O=C1COC[C@]2(CCCN3C(=O)COc4c(F)cccc4[C@H]4CC[C@H](CC4)OC[C@@H]32)N1] | (1′s,3R,16′S,19′s)-6′-fluoro- 8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 2 | C |
| 39a | 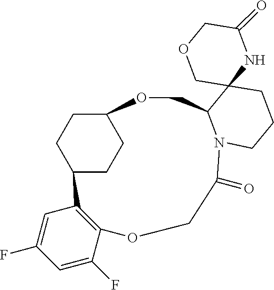 [SMILES: O=C1COC[C@@]2(CCCN3C(=O)COc4c(F)cc(F)cc4[C@H]4CC[C@H](CC4)OC[C@H]32)N1] | (1′s,3S,16′R,19′s)-4′,6′- difluoro-8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 1 | D |
| 39b | 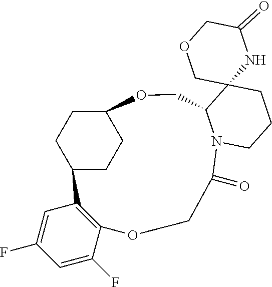 [SMILES: O=C1COC[C@]2(CCCN3C(=O)COc4c(F)cc(F)cc4[C@H]4CC[C@H](CC4)OC[C@@H]32)N1] | (1′s,3R,16′S,19′s)-4′,6′- difluoro-8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 2 | A |
| 40a | 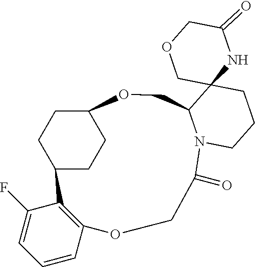 [SMILES: O=C1COC[C@@]2(CCCN3C(=O)COc4cccc(F)c4[C@H]4CC[C@H](CC4)OC[C@H]32)N1] | (1′s,3S,16′R,19′s)-3′-fluoro- 8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 1 | A |
| 40b | 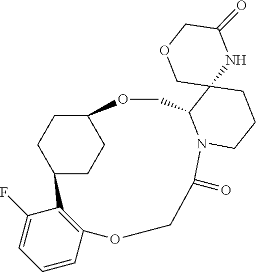 [SMILES: O=C1COC[C@]2(CCCN3C(=O)COc4cccc(F)c4[C@H]4CC[C@H](CC4)OC[C@@H]32)N1] | (1′s,3R,16′S,19′s)-3′-fluoro- 8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 2 | C |
| 41 | 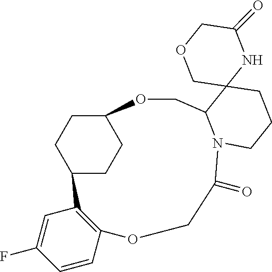 [SMILES: O=C1COCC2(CCCN3C(=O)COc4ccc(F)cc4[C@H]4CC[C@H](CC4)OCC32)N1] | Rel-(1′s,3S,16′R,19′s)-4′- fluoro-8′,18′-dioxa-11′- azaspiro[morpholine-3,15′- tetracyclo[17.2.2.02,7.011,16] tricosane]- 2′(7′),3′,5′-triene-5,10′-dione | Racemic- CIS | A |
| 41a | 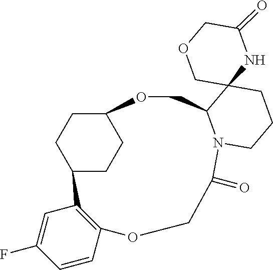 [SMILES: O=C1COC[C@@]2(CCCN3C(=O)COc4ccc(F)cc4[C@H]4CC[C@H](CC4)OC[C@H]32)N1] | (1′s,3S,16′R,19′s)-4′-fluoro- 8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 1 | A |
| 41b | 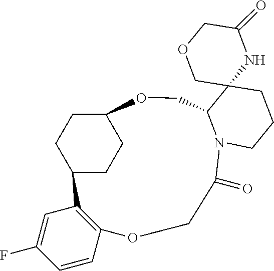 [SMILES: O=C1COC[C@]2(CCCN3C(=O)COc4ccc(F)cc4[C@H]4CC[C@H](CC4)OC[C@@H]32)N1] | (1′s,3R,16′S,19′s)-4′-fluoro- 8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 2 | C |
| 42a | 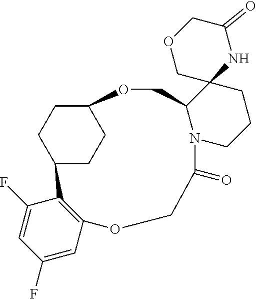 [SMILES: O=C1COC[C@@]2(CCCN3C(=O)COc4cc(F)cc(F)c4[C@H]4CC[C@H](CC4)OC[C@H]32)N1] | (1′s,3S,16′R,19′s)-3′,5′- difluoro-8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 1 | A |
| 42b | 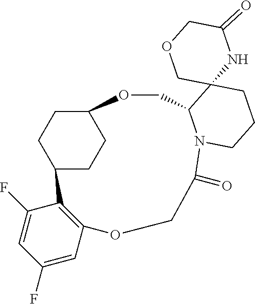 [SMILES: O=C1COC[C@]2(CCCN3C(=O)COc4cc(F)cc(F)c4[C@H]4CC[C@H](CC4)OC[C@@H]32)N1] | (1′s,3R,16′S,19′s)-3′,5′- difluoro-8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 2 | B |
| 43a | 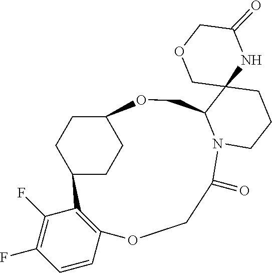 [SMILES: O=C1COC[C@@]2(CCCN3C(=O)COc4ccc(F)c(F)c4[C@H]4CC[C@H](CC4)OC[C@H]32)N1] | (1′s,3S,16′R,19′s)-3′,4′- difluoro-8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 1 | A |
| 43b | 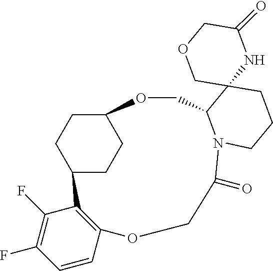 [SMILES: O=C1COC[C@]2(CCCN3C(=O)COc4ccc(F)c(F)c4[C@H]4CC[C@H](CC4)OC[C@@H]32)N1] | (1′s,3R,16′S,19′s)-3′,4′- difluoro-8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 2 | D |
| 44a | 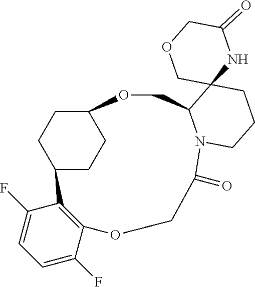 [SMILES: O=C1COC[C@@]2(CCCN3C(=O)COc4c(F)ccc(F)c4[C@H]4CC[C@H](CC4)OC[C@H]32)N1] | (1′s,3S,16′R,19′s)-3′,6′- difluoro-8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 1 | A |
| 44b | 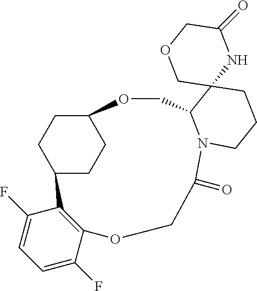 [SMILES: O=C1COC[C@]2(CCCN3C(=O)COc4c(F)ccc(F)c4[C@H]4CC[C@H](CC4)OC[C@@H]32)N1] | (1′s,3R,16′S,19′s)-3′,6′- difluoro-8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 2 | D |
| 45 | 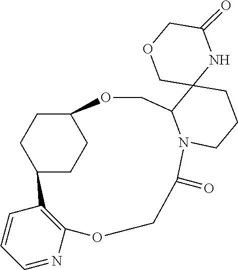 [SMILES: O=C1COCC2(CCCN3C(=O)COc4ncccc4[C@H]4CC[C@H](CC4)OCC32)N1] | Rel-(1′s,3S,16′R,19′s)-8′,18′- dioxa-6′,11′- diazaspiro[morpholine-3,15′- tetracyclo[17.2.2.02,7.011,16] tricosane]- 2′(7′),3′,5′-triene-5,10′-dione | Racemic- CIS | A |
| 45a | 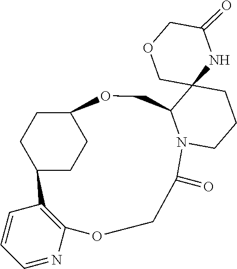 [SMILES: O=C1COC[C@@]2(CCCN3C(=O)COc4ncccc4[C@H]4CC[C@H](CC4)OC[C@H]32)N1] | (1′s,3S,16′R,19′s)-8′,18′- dioxa-6′,11′- diazaspiro[morpholine-3,15′- tetracyclo[17.2.2.0²,⁷.0¹¹,¹⁶] tricosane]-2′(7′),3′,5′-triene- 5,10′-dione | Isomer 1 | A |
| 45b | 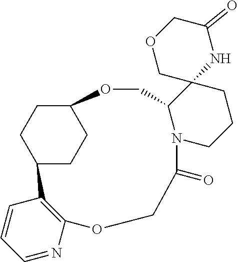 [SMILES: O=C1COC[C@]2(CCCN3C(=O)COc4ncccc4[C@H]4CC[C@H](CC4)OC[C@@H]32)N1] | (1′s,3R,16′S,19′s)-8′,18′- dioxa-6′,11′- diazaspiro[morpholine-3,15′- tetracyclo[17.2.2.0²,⁷.0¹¹,¹⁶] tricosane]-2′(7′),3′,5′-triene- 5,10′-dione | Isomer 2 | D |
| 46a | 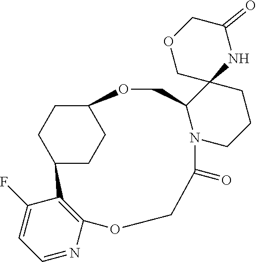 [SMILES: O=C1COC[C@@]2(CCCN3C(=O)COc4nccc(F)c4[C@H]4CC[C@H](CC4)OC[C@H]32)N1] | (1′s,3S,16′R,19′s)-3′-fluoro- 8′,18′-dioxa-6′,11′- diazaspiro[morpholine-3,15′- tetracyclo[17.2.2.0²,⁷.0¹¹,¹⁶] tricosane]-2′(7′),3′,5′-triene- 5,10′-dione | Isomer 1 | A |
| 46b | 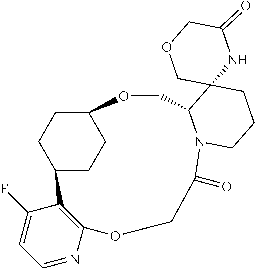 [SMILES: O=C1COC[C@]2(CCCN3C(=O)COc4nccc(F)c4[C@H]4CC[C@H](CC4)OC[C@@H]32)N1] | (1′s,3R,16′S,19′s)-3′-fluoro- 8′,18′-dioxa-6′,11′- diazaspiro[morpholine-3,15′- tetracyclo[17.2.2.0²,⁷.0¹¹,¹⁶] tricosane]-2′(7′),3′,5′-triene- 5,10′-dione | Isomer 2 | C |
| 47 | 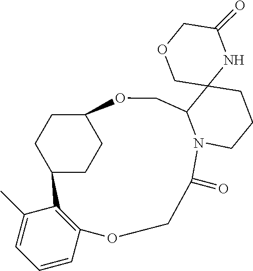 [SMILES: Cc1cccc2c1[C@H]1CC[C@H](CC1)OCC1N(CCCC13COCC(=O)N3)C(=O)CO2] | Rel-(1′s,3S,16′R,19′s)-3′- methyl-8′,18′-dioxa-11′- azaspiro[morpholine-3,15′- tetracyclo[17.2.2.02,7.011,16] tricosane]- 2′(7′),3′,5′-triene-5,10′-dione | Racemic - CIS | A |
| 47a | 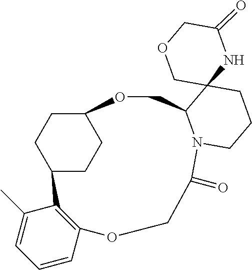 [SMILES: Cc1cccc2c1[C@H]1CC[C@H](CC1)OC[C@@H]1N(CCC[C@@]13COCC(=O)N3)C(=O)CO2] | (1′s,3S,16′R,19′s)-3′-methyl- 8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 1 | A |
| 47b | 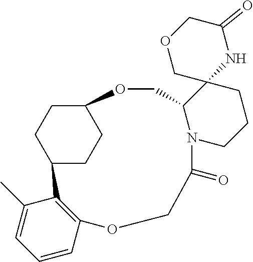 [SMILES: Cc1cccc2c1[C@H]1CC[C@H](CC1)OC[C@H]1N(CCC[C@]13COCC(=O)N3)C(=O)CO2] | (1′s,3R,16′S,19′s)-3′-methyl- 8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 2 | C |
| 48a | 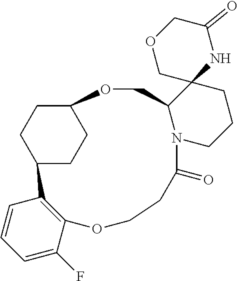 [SMILES: O=C1COC[C@@]2(CCCN3C(=O)CCOc4c(F)cccc4[C@H]4CC[C@H](CC4)OC[C@H]32)N1] | (1′s,3S,17′R,20′s)-6′-fluoro- 8′,19′-dioxa-12′- azaspiro[morpholine-3,16′- tetracyclo[18.2.2.0²,⁷.0¹²,¹⁷] tetracosane]-2′(7′),3′,5′-triene- 5,11′-dione | Isomer 1 | A |
| 48b | 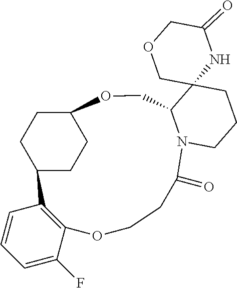 [SMILES: O=C1COC[C@]2(CCCN3C(=O)CCOc4c(F)cccc4[C@H]4CC[C@H](CC4)OC[C@@H]32)N1] | (1′s,3R,17′S,20′s)-6′-fluoro- 8′,19′-dioxa-12′- azaspiro[morpholine-3,16′- tetracyclo[18.2.2.0²,⁷.0¹²,¹⁷] tetracosane]-2′(7′),3′,5′-triene- 5,11′-dione | Isomer 2 | A |
| 49 | 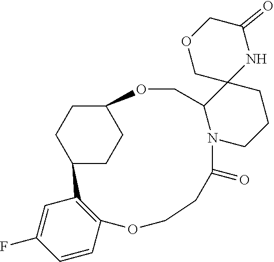 [SMILES: O=C1COCC2(CCCN3C(=O)CCOc4ccc(F)cc4[C@H]4CC[C@H](CC4)OCC32)N1] | Rel-(1′s,3S,17′R,20′s)-4′- fluoro-8′,19′-dioxa-12′- azaspiro[morpholine-3,16′- tetracyclo[18.2.2.02,7.012,17] tetracosane]- 2′(7′),3′,5′-triene-5,11′-dione | Racemic- CIS | A |
| 49a | 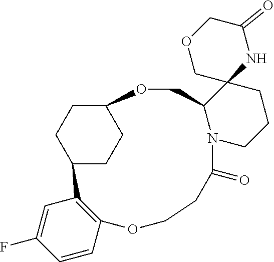 [SMILES: O=C1COC[C@@]2(CCCN3C(=O)CCOc4ccc(F)cc4[C@H]4CC[C@H](CC4)OC[C@H]32)N1] | (1′s,3S,17′R,20′s)-4′-fluoro- 8′,19′-dioxa-12′- azaspiro[morpholine-3,16′- tetracyclo[18.2.2.0²,⁷.0¹²,¹⁷] tetracosane]-2′(7′),3′,5′-triene- 5,11′-dione | Isomer 1 | A |
| 49b | 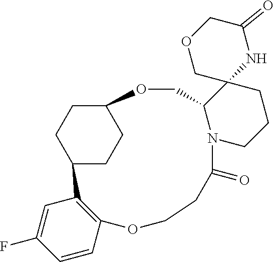 [SMILES: O=C1COC[C@]2(CCCN3C(=O)CCOc4ccc(F)cc4[C@H]4CC[C@H](CC4)OC[C@@H]32)N1] | (1′s,3R,17′S,20′s)-4′-fluoro- 8′,19′-dioxa-12′- azaspiro[morpholine-3,16′- tetracyclo[18.2.2.0²,⁷.0¹²,¹⁷] tetracosane]-2′(7′),3′,5′-triene- 5,11′-dione | Isomer 2 | A |
| 50a | 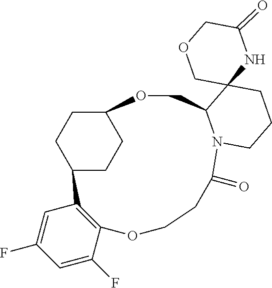 [SMILES: O=C1COC[C@@]2(CCCN3C(=O)CCOc4c(F)cc(F)cc4[C@H]4CC[C@H](CC4)OC[C@H]32)N1] | (1′s,3S,17′R,20′s)-4′,6′- difluoro-8′,19′-dioxa-12′- azaspiro[morpholine-3,16′- tetracyclo[18.2.2.0²,⁷.0¹²,¹⁷] tetracosane]-2′(7′),3′,5′-triene- 5,11′-dione | Isomer 1 | A |
| 50b | 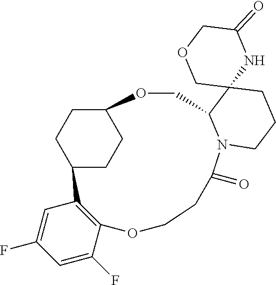 [SMILES: O=C1COC[C@]2(CCCN3C(=O)CCOc4c(F)cc(F)cc4[C@H]4CC[C@H](CC4)OC[C@@H]32)N1] | (1′s,3R,17′S,20′s)-4′,6′- difluoro-8′,19′-dioxa-12′- azaspiro[morpholine-3,16′- tetracyclo[18.2.2.0²,7.0¹²,¹⁷] tetracosane]-2′(7′),3′,5′-triene- 5,11′-dione | Isomer 2 | B |
| 51 | 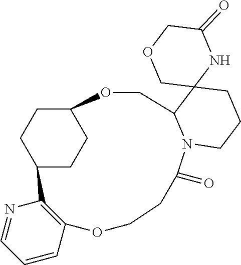 [SMILES: O=C1COCC2(CCCN3C(=O)CCOc4cccnc4[C@H]4CC[C@H](CC4)OCC32)N1] | Rel-(1′s,3S,17′R,20′s)-8′,19′- dioxa-3′,12′- diazaspiro[morpholine-3,16′- tetracyclo[18.2.2.0²,⁷.0¹²,¹⁷] tetracosane]- 2′(7′),3′,5′-triene-5,11′-dione | Racemic- CIS | D |
| 52 | 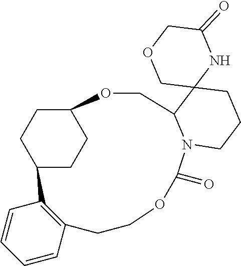 [SMILES: O=C1COCC2(CCCN3C(=O)OCCc4ccccc4[C@H]4CC[C@H](CC4)OCC32)N1] | Rel-(1′s,3S,17′R,20′s)-10′,19′- dioxa-12′- azaspiro[morpholine-3,16′- tetracyclo[18.2.2.02,7.012,17] tetracosane]- 2′(7′),3′,5′-triene-5,11′-dione | Racemic- CIS | C |
| 53 | 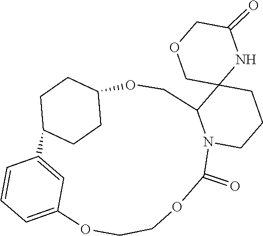 [SMILES: O=C1COCC2(CCCN3C(=O)OCCOc4cccc(c4)[C@H]4CC[C@H](CC4)OCC32)N1] | Rel-(1′s,3S,17′R,20′s)- 7′,10′,19′-trioxa-12′- azaspiro[morpholine-3,16′- tetracyclo[18.2.2.12,6.012,17 ]pentacosane]-2′(25′),3′,5′- triene-5,11′-dione | Racemic- CIS | A |
| 53a | 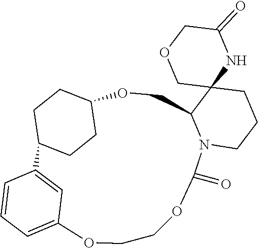 [SMILES: O=C1COC[C@@]2(CCCN3C(=O)OCCOc4cccc(c4)[C@H]4CC[C@H](CC4)OC[C@H]32)N1] | (1′s,3S,17′R,20′s)-7′,10′,19′- trioxa-12′- azaspiro[morpholine-3,16′- tetracyclo[18.2.2.1²,⁶.0¹²,¹⁷] pentacosane]- 2′(25′),3′,5′-triene-5,11′-dione | Isomer 1 | A |
| 53b | 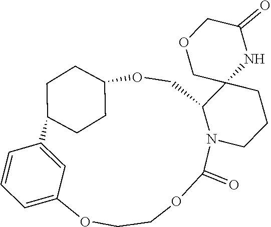 [SMILES: O=C1COC[C@]2(CCCN3C(=O)OCCOc4cccc(c4)[C@H]4CC[C@H](CC4)OC[C@@H]32)N1] | (1′s,3R,17′S,20′s)-7′,10′,19′- trioxa-12′- azaspiro[morpholine-3,16′- tetracyclo[18.2.2.1^{2,6}.0^{12,17}] pentacosane]- 2′(25′),3′,5′-triene-5,11′-dione | Isomer 2 | D |
| 54 | 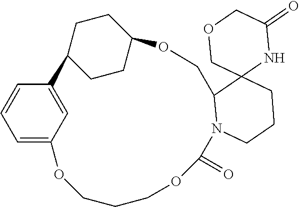 [SMILES: O=C1COCC2(CCCN3C(=O)OCCCOc4cccc(c4)[C@H]4CC[C@H](CC4)OCC32)N1] | Rel-(1′s,3S,18′R,21′s)- 7′,11′,20′-trioxa-13′- azaspiro[morpholine-3,17′- tetracyclo[19.2.2.12,6.013,18] hexacosane]-2′(26′),3′,5′- triene-5,12′-dione | Racemic- CIS | A |
| 54a | 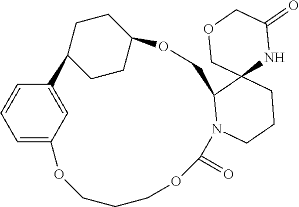 [SMILES: O=C1COC[C@@]2(CCCN3C(=O)OCCCOc4cccc(c4)[C@H]4CC[C@H](CC4)OC[C@H]32)N1] | (1′s,3S,18′R,21′s)-7′,11′,20′- trioxa-13′- azaspiro[morpholine-3,17′- tetracyclo[19.2.2.12,6.013,18] hexacosane]-2′(26′),3′,5′- triene-5,12′-dione | Isomer 1 | A |
| 54b | 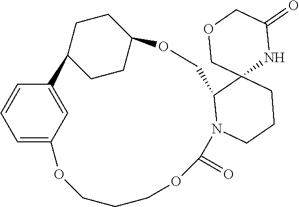 [SMILES: O=C1COC[C@]2(CCCN3C(=O)OCCCOc4cccc(c4)[C@H]4CC[C@H](CC4)OC[C@@H]32)N1] | (1′s,3R,18′S,21′s)-7′,11′,20′- trioxa-13′- azaspiro[morpholine-3,17′- tetracyclo[19.2.2.12,6.013,18] hexacosane]-2′(26′),3′,5′- triene-5,12′-dione | Isomer 2 | C |
| 55 | 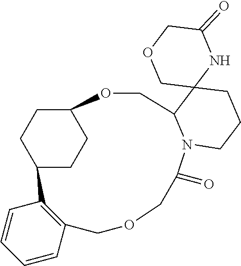 [SMILES: O=C1COCC2(CCCN3C(=O)COCc4ccccc4[C@H]4CC[C@H](CC4)OCC32)N1] | Rel-(1′s,3S,17′R,20′s)-9′,19′- dioxa-12′- azaspiro[morpholine-3,16′- tetracyclo[18.2.2.02,7.012,17] tetracosane]- 2′(7′),3′,5′-triene-5,11′-dione | Racemic- CIS | C |
| 56a | 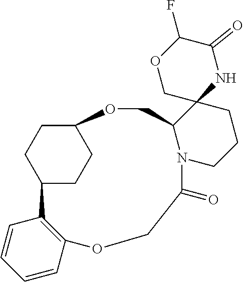 | (1′s,3S,16′R,19′s)-6-fluoro- 8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 1 | A |
| 56b | 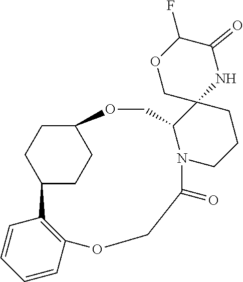 | (1′s,3R,16′S,19′s)-6-fluoro- 8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 2 | A |
| 57 | 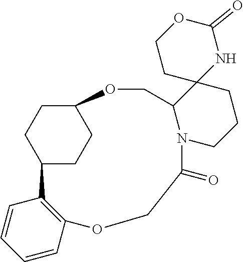 [SMILES: O=C1NC2(CCCN3C(=O)COc4ccccc4[C@H]4CC[C@H](CC4)OCC32)CCO1] | Rel-(1′s,4R,16′R,19′s)-8′,18′- dioxa-11′-azaspiro[1,3- oxazinane-4,15′- tetracyclo[17.2.2.0²,⁷.0¹¹,¹⁶] tricosane]-2′(7′),3′,5′-triene- 2,10′-dione | Racemic - CIS | A |
| 58 | 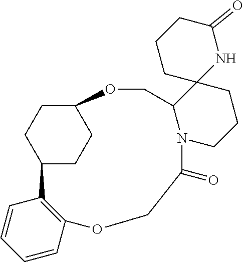 [SMILES: O=C1CCCC2(CCCN3C(=O)COc4ccccc4[C@H]4CC[C@H](CC4)OCC32)N1] | Rel-(1′s,2S,16′R,19′s)-8′,18′- dioxa-11′- azaspiro[piperidine-2,15′- tetracyclo[17.2.2.0²,⁷.0¹¹,¹⁶] tricosane]-2′(7′),3′,5′-triene- 6,10′-dione | Racemic - CIS | A |
| 58a | 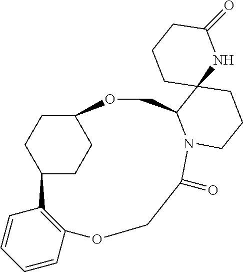 [SMILES: O=C1CCC[C@@]2(CCCN3C(=O)COc4ccccc4[C@H]4CC[C@H](CC4)OC[C@H]32)N1] | (1′s,2S,16′R,19′s)-8′,18′- dioxa-11′- azaspiro[piperidine-2,15′- tetracyclo[17.2.2.0²,⁷.0¹¹,¹⁶] tricosane]-2′(7′),3′,5′-triene- 6,10′-dione | Isomer 1 | A |
| 58b | 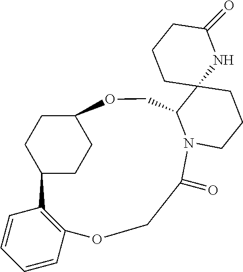 [SMILES: O=C1CCC[C@]2(CCCN3C(=O)COc4ccccc4[C@H]4CC[C@H](CC4)OC[C@@H]32)N1] | (1′s,2R,16′S,19′s)-8′,18′- dioxa-11′- azaspiro[piperidine-2,15′- tetracyclo[17.2.2.0²,⁷.0¹¹,¹⁶] tricosane]-2′(7′),3′,5′-triene- 6,10′-dione | Isomer 2 | D |
| 59 | 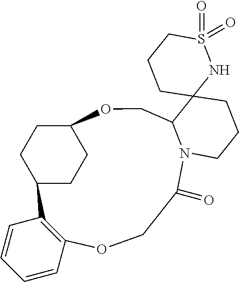 [SMILES: O=C1COc2ccccc2[C@H]2CC[C@H](CC2)OCC2N1CCCC21CCCS(=O)(=O)N1] | Rel-(1s,15R,16R,19s)-8,18- dioxa-11- azaspiro[tetracyclo[17.2.2.0², ⁷.0¹¹,¹⁶]tricosane-15,3′- [1λ⁶,2]thiazinane]-2(7),3,5- triene-1′,1′,10-trione | Racemic- CIS | A |
| 59a | 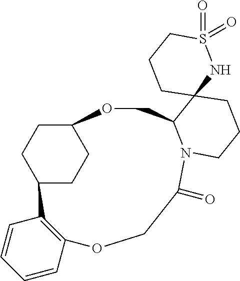 [SMILES: O=C1COc2ccccc2[C@H]2CC[C@H](CC2)OC[C@@H]2N1CCC[C@@]21CCCS(=O)(=O)N1] | (1s,15R,16R,19s)-8,18- dioxa-11- azaspiro[tetracyclo [17.2.2.0²,⁷.0¹¹,¹⁶] tricosane-15,3′- [1λ⁶,2]thiazinane]-2(7),3,5- triene-1′,1′,10-trione | Isomer 1 | A |
| 59b | 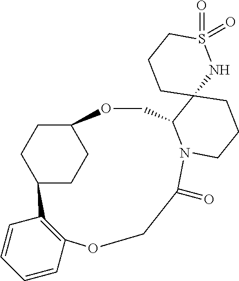 [SMILES: O=C1COc2ccccc2[C@H]2CC[C@H](CC2)OC[C@H]2N1CCC[C@]21CCCS(=O)(=O)N1] | (1s,15S,16S,19s)-8,18-dioxa-11- azaspiro[tetracyclo [17.2.2.0²,⁷.0¹¹,¹⁶] tricosane-15,3′- [1λ⁶,2]thiazinane]-2(7),3,5- triene-1′,1′,10-trione | Isomer 2 | C |
| 60 | 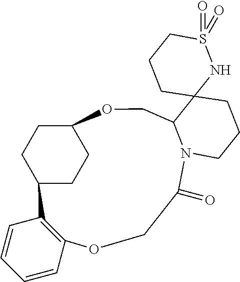 [SMILES: O=C1COc2ccccc2[C@H]2CC[C@H](CC2)OCC2N1CCCC21CCCS(=O)(=O)N1] | Rel-(1s,16R,17R,20s)-8,19- dioxa-12- azaspiro[tetracyclo [18.2.2.0²,⁷.0¹²,¹⁷] tetracosane-16,3′- [126,2]thiazinane]-2(7),3,5- triene-1′,1′,11-trione | Racemic - CIS | B |
| 61 | 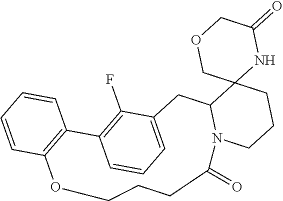 [SMILES: O=C1COCC2(CCCN3C(=O)CCCOc4ccccc4-c4cccc(c4F)CC32)N1] | Rel-(3S,18′S)-24′-fluoro-8′- oxa-13′-azaspiro[morpholine- 3,17′- tetracyclo[18.3.1.02,7.013,18] tetracosane]- 1′(24′),2′(7′),3′,5′,20′,22′- hexaene-5,12′-dione | Racemic - CIS | C |
| 62 | 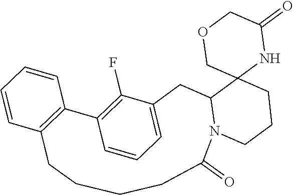 [SMILES: O=C1COCC2(CCCN3C(=O)CCCCc4ccccc4-c4cccc(c4F)CC32)N1] | Rel-(3S,18′S)-24′-fluoro-13′- azaspiro[morpholine-3,17′- tetracyclo[18.3.1.02,7.013,18 tetracosane]- l′(24′),2′(7′),3′,5′,20′,22′- hexaene-5,12′-dione | Racemic CIS | D |
| 63 | 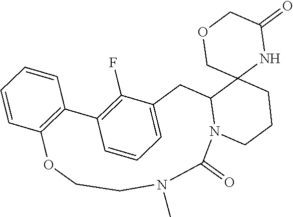 [SMILES: CN1CCOc2ccccc2-c2cccc(c2F)CC2N(CCCC23COCC(=O)N3)C1=O] | Rel-(3S,18′S)-24′-fluoro-11′- methyl-8′-oxa-11′,13′- diazaspiro[morpholine-3,17′- tetracyclo[18.3.1.02,7.013,18] tetracosane]- 1′(24′),2′(7′),3′,5′,20′,22′- hexaene-5,12′-dione | Racemic - CIS | C |
| 64 | 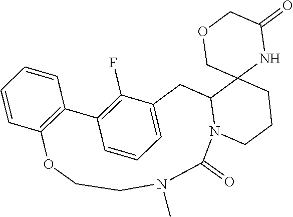 [SMILES: CN1CCOc2ccccc2-c2cccc(c2F)CC2N(CCCC23COCC(=O)N3)C1=O] | Rel-(3S,19′S)-25′-fluoro-12′- methyl-8′-oxa-12′,14′- diazaspiro[morpholine-3,18′- tetracyclo[19.3.1.02,7.014,19] pentacosane]- 1′(25′),2′(7′),3′,5′,21′,23′- hexaene-5,13′-dione | Racemic - CIS | D |
| 65 | 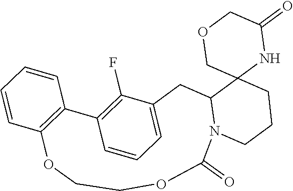 [SMILES: O=C1COCC2(CCCN3C(=O)OCCOc4ccccc4-c4cccc(c4F)CC32)N1] | Rel-(3S,18′S)-24′-fluoro- 8′,11′-dioxa-13′- azaspiro[morpholine-3,17′- tetracyclo[18.3.1.0²,⁷.0¹³,¹⁸] tetracosane]- l′(24′),2′(7′),3′,5′,20′,22′- hexaene-5,12′-dione | Racemic - CIS | D |
| 66 | 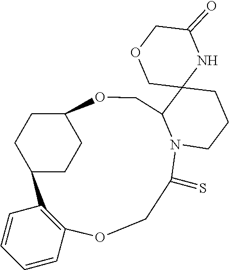 [SMILES: O=C1COCC2(CCCN3C(=S)COc4ccccc4[C@H]4CC[C@H](CC4)OCC32)N1] | Rel-(1′s,3S,16′R,19′s)-10′- sulfanylidene-8′,18′-dioxa- 11′-azaspiro[morpholine- 3,15′-tetracyclo [17.2.2.0²,⁷.0¹¹,¹⁶]tricosane]- 2′(7′),3′,5′-trien-5-one | Racemic - CIS | C |
| 67a | 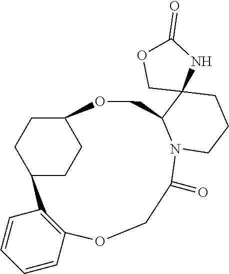 [SMILES: O=C1N[C@]2(CCCN3C(=O)COc4ccccc4[C@H]4CC[C@H](CC4)OC[C@H]32)CO1] | (1′s,3S,16′R,19′s)-8′,18′- dioxa-11′-azaspiro[1,4- oxazolidine-3,15′- tetracyclo[17.2.2.0²,⁷.0¹¹,¹⁶] tricosane]-2′(7′),3′,5′-triene- 5,10′-dione | Isomer 1 | A |
| 67b | 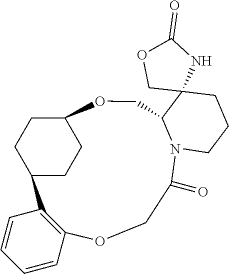 [SMILES: O=C1N[C@@]2(CCCN3C(=O)COc4ccccc4[C@H]4CC[C@H](CC4)OC[C@@H]32)CO1] | (1′s,3R,16′S,19′s)-8′,18′- dioxa-11′-azaspiro[1,4- oxazolidine-3,15′- tetracyclo[17.2.2.0²,⁷.0¹¹,¹⁶] tricosane]-2′(7′),3′,5′-triene- 5,10′-dione | Isomer 2 | D |
| 68a | 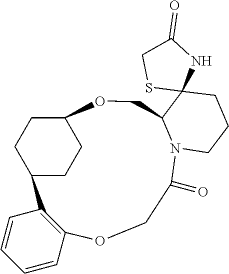 [SMILES: O=C1CS[C@@]2(CCCN3C(=O)COc4ccccc4[C@H]4CC[C@H](CC4)OC[C@H]32)N1] | Rel-(1s,15R,16S,19s)-8,18- dioxa-11- azaspiro[tetracyclo [17.2.2.0²,⁷.0¹¹,¹⁶] tricosane-15,2′- [1,3]thiazolidine]-2(7),3,5- triene-4′,10-dione | Isomer 1 | B |
| 68b | 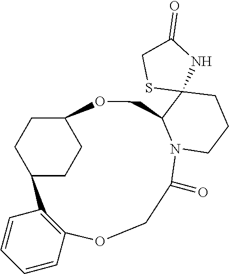 [SMILES: O=C1CS[C@]2(CCCN3C(=O)COc4ccccc4[C@H]4CC[C@H](CC4)OC[C@H]32)N1] | Rel-(1s,15S,16S,19s)-8,18- dioxa-11- azaspiro[tetracyclo [17.2.2.0²,⁷.0¹¹,¹⁶] tricosane-15,2′- [1,3]thiazolidine]-2(7),3,5- triene-4′,10-dione | Isomer 2 | D |
| 69 | 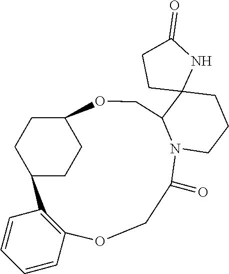 [SMILES: O=C1CCC2(CCCN3C(=O)COc4ccccc4[C@H]4CC[C@H](CC4)OCC32)N1] | Rel-(1′s,2R,16′R,19′s)-8′,18′- dioxa-11′- azaspiro[pyrrolidine-2,15′- tetracyclo[17.2.2.0²,⁷.0¹¹,¹⁶] tricosane]-2′(7′),3′,5′-triene- 5,10′-dione | Racemic - CIS | B |
| 70 | 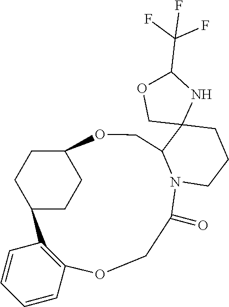 [SMILES: O=C1COc2ccccc2[C@H]2CC[C@H](CC2)OCC2N1CCCC21COC(C(F)(F)F)N1] | Rel-(1′s,3S,16′R,19′s)-5- (trifluoromethyl)-8′,18′- dioxa-11′-azaspiro[1,4- oxazolidine-3,15′-tetracyclo [17.2.2.0²,⁷.0¹¹,¹⁶]tricosane]- 2′(7′),3′,5′-trien-10′-one | Racemic - CIS | B |
| 71a | 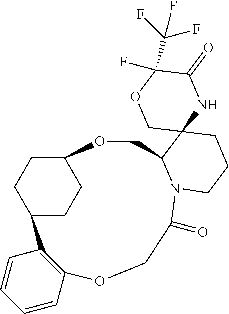 [SMILES: O=C1COc2ccccc2[C@H]2CC[C@H](CC2)OC[C@@H]2N1CCC[C@@]21CO[C@](F)(C(F)(F)F)C(=O)N1] | Rel-(1′s,3S,16′R,19′s)-6(R)- fluoro-6-(trifluoromethyl)- 8′,18′-dioxa-11′- azaspiro[morpholine-3,15′- tetracyclo[17.2.2.0²,⁷.0¹¹,¹⁶] tricosane]-2′(7′),3′,5′-triene- 5,10′-dione | Isomer 1 | A |
| 71b | 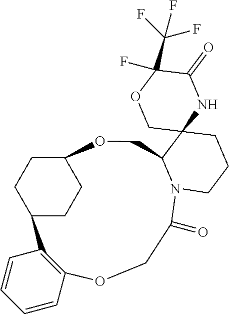 | Rel-(1′s,3S,16′R,19′s)-6(S)- fluoro-6-(trifluoromethyl)- 8′,18′-dioxa-11′- azaspiro[morpholine-3,15′- tetracyclo[17.2.2.0²,⁷.0¹¹,¹⁶] tricosane]- 2′(7′),3′,5′-triene-5,10′-dione | Isomer 2 | A |
| 72 | 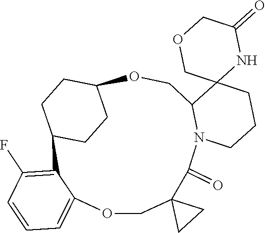 [SMILES: O=C1COCC2(CCCN3C(=O)C4(CC4)COc4cccc(F)c4[C@H]4CC[C@H](CC4)OCC32)N1] | (1′s,20′s)-3′- fluorodispiro[cyclopropane- 1,10′-[8,19]dioxa- [12]azatetracyclo[18.2.2.0²,⁷. 0¹²,¹⁷]tetracosane-16′,3″- morpholine]-2′,4′,6′-triene- 5″,11′-dione | Racemic - CIS | A |
| 72a | 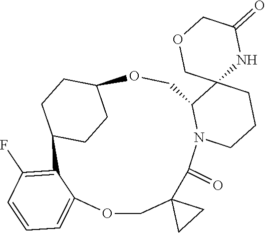 [SMILES: O=C1COC[C@]2(CCCN3C(=O)C4(CC4)COc4cccc(F)c4[C@H]4CC[C@H](CC4)OC[C@@H]32)N1] | (1′s,16′R,17′S,20′s)-3′- fluorodispiro[cyclopropane- 1,10′-[8,19]dioxa- [12]azatetracyclo[18.2.2.0²,⁷. 0¹²,¹⁷]tetracosane-16′,3″- morpholine]-2′,4′,6′-triene- 5″,11′-dione | Isomer 1 | C |
| 72b | 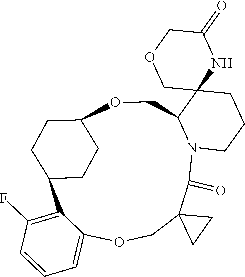 [SMILES: O=C1COC[C@@]2(CCCN3C(=O)C4(CC4)COc4cccc(F)c4[C@H]4CC[C@H](CC4)OC[C@H]32)N1] | (1′s,16′S,17′R,20′s)-3′- fluorodispiro[cyclopropane- 1,10′-[8,19]dioxa- [12]azatetracyclo[18.2.2.0²,⁷. 0¹²,¹⁷]tetracosane-16′,3″- morpholine]-2′,4′,6′-triene- 5″,11′-dione | Isomer 2 | A |
| 73a | 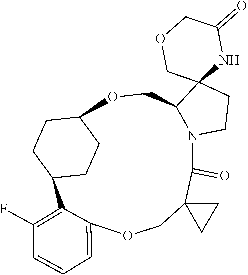 [SMILES: O=C1COC[C@@]2(CCN3C(=O)C4(CC4)COc4cccc(F)c4[C@H]4CC[C@H](CC4)OC[C@H]32)N1] | (1′s,15′S,16′R,19′s)-3′- fluorodispiro[cyclopropane- 1,10′-[8,18]dioxa- [12]azatetracyclo [17.2.2.0²,⁷.0¹²,¹⁶] tricosane-15′,3″- morpholine]-2′,4′,6′-triene- 5″,11′-dione | Isomer 1 | C |
| 73b | 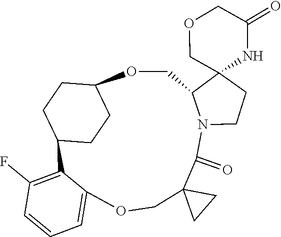 [SMILES: O=C1COC[C@]2(CCN3C(=O)C4(CC4)COc4cccc(F)c4[C@H]4CC[C@H](CC4)OC[C@@H]32)N1] | (1′s,15′R,16′S,19′s)-3′- fluorodispiro[cyclopropane- 1,10′-[8,18]dioxa- [12]azatetracyclo [17.2.2.0²,⁷.0¹²,¹⁶] tricosane-15′,3″- morpholine]-2′,4′,6′-triene- 5″,11′-dione | Isomer 2 | D |
| 74 | 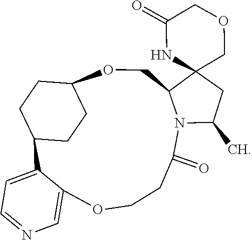 | (1′s,3S,13′R,16′R,19′s)-13′- methy1-8′,18′-dioxa- 5′,12′-diazaspiro[morpholine- 3,15′-tetracyclo [17.2.2.0²,⁷.0¹²,¹⁶] tricosane]- 2′,4′,6′-triene-5,11′-dione | Isomer 1 | A |
| 75 | 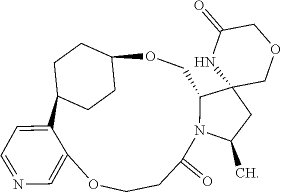 | (1′s,3R,13′R,16′S,19′s)-13′- methy1-8′,18′-dioxa- 5′,12′-diazaspiro[morpholine- 3,15′-tetracyclo [17.2.2.0²,⁷.0¹²,¹⁶] tricosane]- 2′,4′,6′-triene-5,1l′-dione | Isomer 2 | D |
| 76 | 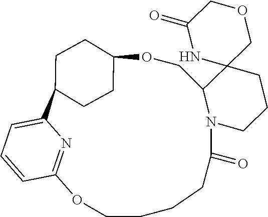 [SMILES: O=C1COCC2(CCCN3C(=O)CCCCOc4cccc(n4)[C@H]4CC[C@H](CC4)OCC32)N1] | (1′s,21′s)-7′,20′-dioxa-13′,26′- diazaspiro[morpholine-3,17′- tetracyclo[19.2.2.1²,⁶.0¹³,¹⁸] hexacosane]-2′,4′,6′(26′)-triene- 5,12′-dione | Racemic - CIS | A |
| 76a | 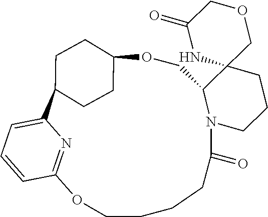 [SMILES: O=C1COC[C@]2(CCCN3C(=O)CCCCOc4cccc(n4)[C@H]4CC[C@H](CC4)OC[C@@H]32)N1] | (1′s,3R,18′S,21′s)-7′,20′- dioxa-13′,26′- diazaspiro[morpholine-3,17′- tetracyclo[19.2.2.1²,⁶.0¹³,¹⁸] hexacosane]-2′,4′,6′(26′)-triene- 5,12′-dione | Isomer 1 | D |
| 76b | 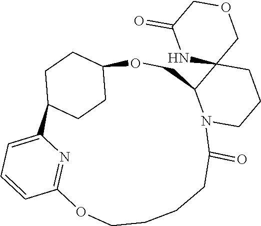 [SMILES: O=C1COC[C@@]2(CCCN3C(=O)CCCCOc4cccc(n4)[C@H]4CC[C@H](CC4)OC[C@H]32)N1] | (1′s,3S,18′R,21′s)-7′,20′- dioxa-13′,26′- diazaspiro[morpholine-3,17′- tetracyclo[19.2.2.1²,⁶.0¹³,¹⁸] hexacosane]-2′,4′,6′(26′)-triene- 5,12′-dione | Isomer 2 | A |
| 77 | 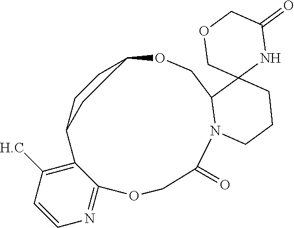 | (1′s,19′s)-3′-methyl-8′,18′- dioxa-6′,11′- diazaspiro[morpholine-3,15′- tetracyclo[17.2.2.0²,⁷.0¹²,¹⁶] tricosane]-2′,4′,6′-triene-5,10′- dione | Racemic - CIS | A |
| 77a | 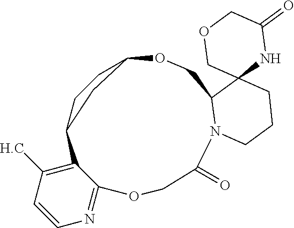 | (1′s,3S,16′R,19′s)-3′-methyl- 8′,18′-dioxa-6′,11′- diazaspiro[morpholine-3,15′- tetracyclo[17.2.2.0²,⁷.0¹¹,¹⁶] tricosane]- 2′,4′,6′-triene-5,10′-dione | Isomer 1 | A |
| 77b | 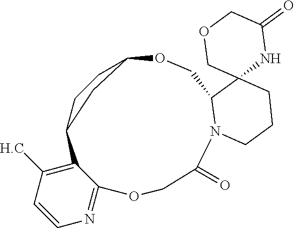 | (1′s,3R,16′S,19′s)-3′-methyl- 8′,18′-dioxa-6′,11′- diazaspiro[morpholine-3,15′- tetracyclo[17.2.2.0²,⁷.0¹²,¹⁶] tricosane]- 2′,4′,6′-triene-5,10′-dione | Isomer 2 | D |

1. A compound of Formula (I): 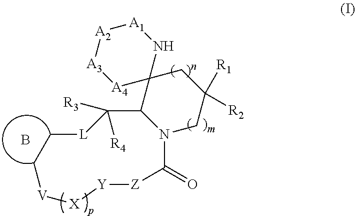
or a pharmaceutically acceptable salt or stereoisomer thereof,
wherein:
L is a linker selected from the group consisting of an optionally substituted aryl, heteroaryl, -carbocyclyl-O—, and -heterocyclyl-O—, wherein-carbocyclyl-O— and -heterocyclyl-O— have the following orientation: 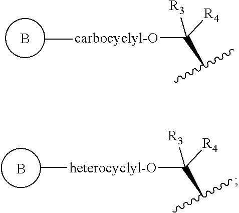
A₁ is —C(O)—, —S(O)₂—, or —C(H)(CF₃)—;
A₂ and A₃ are each independently a bond, —O—, —CR₅R₆—, —NR₇—, or —S—; or A₂ and A₃ together with an optionally substituted carbon atom form a cyclopropyl ring having the structure: 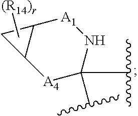
A₄ is a bond, —O—, —CR₅R₆—, —NR₇—, —S—, —(CR₅R₆)₂—, —CR₅R₆—O—, —CR₅R₆—S—, —CR₅R₆—N(R₇)—, —O—CR₅R₆—, —S—CR₅R₆—, or —N(R₇)—CR₅R₆—, with the proviso that the ring that includes A₂, A₃ and A₄ does not contain —O—O—, —O—NR₇— or —NR₇—NR₇—;
\textcircled{B} is phenyl, 5- or 6-membered heteroaryl, cycloalkyl, or heterocyclyl, each of which is optionally substituted;
V and Z are each independently —O—, —CR₈R₉—, or —NR₁₀—;
X is —O—, —CR₁₁R₁₂—, or —NR₁₃—;
Y is a bond, —O—, —CR₈R₉—, or —NR₁₀—;
R₁, R₂, R₃, R₄, R₅, R₆, R₈, R₉, R₁₁, R₁₂ and R₁₄ are each independently hydrogen, halogen, alkyl, cycloalkyl, or heterocyclyl; and/or R₁ and R₂ together with the atom to which they are attached form an optionally substituted carbocycle or heterocycle; and/or R₃ and R₄ together with the atom to which they are attached form an optionally substituted carbocycle or heterocycle; and/or R₅ and R₆ together with the atom to which they are attached form an optionally substituted carbocycle or heterocycle; and/or R₈ and R₉ together with the atom to which they are attached form an optionally substituted carbocycle or heterocycle; and/or R₁₁ and R₁₂ together with the atom to which they are attached form an optionally substituted carbocycle or heterocycle;
R₇, R₁₀, and R₁₃, are each independently hydrogen, alkyl, cycloalkyl, heterocyclyl, aryl, heteroaryl, —(C═O)alkyl, —(C═O)cycloalkyl, —(C═O)heterocyclyl, —(C═O)—O-alkyl, —(C═O)—O-cycloalkyl, —(C—O)—O-heterocyclyl, —(C—O)—O-heteroaryl, —S(O)₂-alkyl, —S(O)₂-cycloalkyl, or —S(O)₂-heterocyclyl; and
m, n, p, and r are each independently 0, 1, or 2.

2. The compound of claim 1, wherein A₁ is —C(O)— or —S(O)₂—.

3. The compound of claim 1 or 2, wherein A₂ is —O— or —CR₅R₆—.

4. The compound of any one of claims 1-3, wherein A₂ is —CR₅R₆—.

5. The compound of any one of claims 1-4, wherein A₃ is —O— or —CR₅R₆—.

6. The compound of any one of claims 1-5, wherein A₃ is —O—.

7. The compound of any one of claims 1-6, wherein A₄ is a bond or —CR₅R₆—.

8. The compound of any one of claims 1-7, wherein R₅ and R₆ are each independently H, halogen, or alkyl.

9. The compound of any one of claims 1-8, wherein R₅ and R₆ are each independently H or alkyl.

10. The compound of claim 8 or 9, wherein the alkyl is methyl, ethyl, or CF₃.

11. The compound of any one of claims 1-9, wherein R₅ and R₆ are H.

12. The compound of any one of claims 1-8, wherein R₅ and R₆ are halogen.

13. The compound of any one of claims 1-8, wherein R₅ and R₆ together with the carbon atom to which they are attached form a carbocycle or heterocycle.

14. The compound of any one of claims 1-13, wherein the carbocycle is a C₃₋₆ cycloalkyl.

15. The compound of any one of claims 1-14, wherein the heterocycle is a 3- or 6-membered heterocycle.

16. The compound of claim 13 or 15, wherein the heterocycle comprises 1 or 2 heteroatoms selected from the group consisting of N, O, and S.

17. The compound of any one of claims 1-16, wherein R₇ is H or alkyl.

18. The compound of any one of claims 1-17, wherein R₁ and R₂ are each independently H, halogen, or alkyl.

19. The compound of any one of claims 1-18, wherein Riis alkyl and R₂ is H.

20. The compound of claim 18 or 19, wherein the alkyl is methyl or ethyl.

21. The compound of any one of claims 1-18, wherein R₁ and R₂ are H.

22. The compound of any one of claims 1-18, wherein R₁ and R₂ are H or halogen.

23. The compound of claim 18 or 22, wherein the halogen is fluoride.

24. The compound of any one of claims 1-18, wherein R₁ and R₂ together with the carbon atom to which they are attached form a carbocycle or heterocycle.

25. The compound of claim 24, wherein the carbocycle is a C₃₋₆ cycloalkyl.

26. The compound of claim 24 or 25, wherein the heterocycle is a 3- or 6-membered heterocycle.

27. The compound of claim 24 or 26, wherein the heterocycle comprises 1 or 2 heteroatoms selected from the group consisting of N, O, and S.

28. The compound of any one of claims 1-27, wherein R₃ and R₄ are each independently H, halogen, or alkyl.

29. The compound of any one of claims 1-28, wherein R₃ and R₄ are each independently H or alkyl.

30. The compound of claim 28 or 29, wherein the alkyl is methyl or ethyl.

31. The compound of any one of claims 1-29, wherein R₃ and R₄ are H.

32. The compound of any one of claims 1-28, wherein R₃ and R₄ are halogen.

33. The compound of claim 32, wherein the halogen is fluoride.

34. The compound of any one of claims 1-28, wherein R₃ and R₄ together with the carbon atom to which they are attached form a carbocycle or heterocycle.

35. The compound of claim 34, wherein the carbocycle is a C₃₋₆ cycloalkyl.

36. The compound of claim 34 or 35, wherein the heterocycle is a 3- or 6-membered heterocycle.

37. The compound of claim 34 or 36, wherein the heterocycle comprises 1 or 2 heteroatoms selected from the group consisting of N, O, and S.

38. The compound of any one of claims 1-37, wherein V is —O— or —CR₈R₉—.

39. The compound of any one of claims 1-38, wherein V is —O— or —NR₁₀—.

40. The compound of any one of claims 1-39, wherein V is —O—.

41. The compound of any one of claims 1-38, wherein V is —CR₈R₉—.

42. The compound of any one of claims 1-41, wherein Y is a bond or —CR₈R₉—.

43. The compound of any one of claims 1-42, wherein Z is a —O— or —CR₈R₉—.

44. The compound of any one of claims 1-41, wherein Z is —CR₈R₉—.

45. The compound of any one of claims 1-44, wherein R₈ and R₉ are each independently H or alkyl.

46. The compound of any one of claims 1-44, wherein R₈ and R₉ together with the carbon atom to which they are attached form a C₃₋₆ cycloalkyl.

47. The compound of any one of claims 1-46, wherein R₁₀ is H or alkyl.

48. The compound of claim 45 or 47, wherein the alkyl is methyl, ethyl, or isopropyl.

49. The compound of any one of claims 1-48, wherein X is —CR₁₁R₁₂—.

50. The compound of any one of claims 1-49, wherein R₁₁ and R₁₂ are each independently H or alkyl.

51. The compound of claim 50, wherein the alkyl is methyl or ethyl.

52. The compound of any one of claims 1-49, wherein R₁₁ and R₁₂ together with the carbon atom to which they are attached form a C₃₋₆ cycloalkyl.

53. The compound of any one of claims 1-52, wherein \textcircled{B} is optionally substituted phenyl.

54. The compound of any one of claims 1-53, wherein the optionally substituted phenyl is: 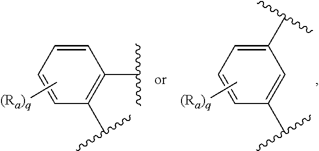
wherein Rₐ is halogen, alkyl, or alkoxy; and q is 0, 1, or 2.

55. The compound of any one of claims 1-52, wherein \textcircled{B} is optionally substituted 5-membered heteroaryl.

56. The compound of any one of claims 1-52, wherein \textcircled{B} is optionally substituted 6-membered heteroaryl.

57. The compound of claim 56, wherein the optionally substituted 6-membered heteroaryl is selected from the group consisting of pyridinyl, pyrazinyl, pyrimidinyl, or pyridazinyl.

58. The compound of claim 56 or 57, wherein the optionally substituted 6-membered heteroaryl is: 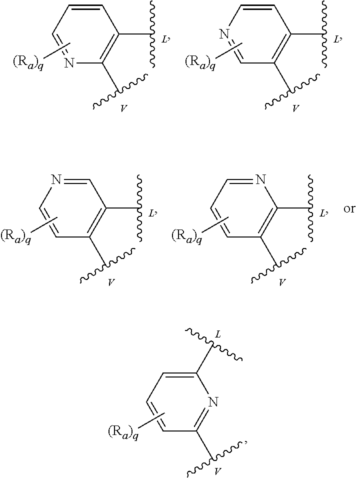
wherein Rₐ is halogen, alkyl, or alkoxy; and q is 0, 1, or 2.

59. The compound of any one of claims 54-58, wherein q is 0 or 1.

60. The compound of any one of claims 54-58, wherein q is 0.

61. The compound of any one of claims 1-60, wherein m is 0 or 1.

62. The compound of any one of claims 1-60, wherein m is 0.

63. The compound of any one of claims 1-62, wherein n is 0 or 1.

64. The compound of any one of claims 1-62, wherein n is 1.

65. The compound of any one of claims 1-64, wherein p is 0 or 1.

66. The compound of any one of claims 1-64, wherein p is 0.

67. The compound of any one of claims 1-64, wherein p is 1.

68. The compound of any one of claims 1-67, wherein L is a -carbocyclyl-O— or -heterocyclyl-O— linker having the structure 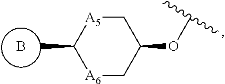
wherein A₅ and A₆ are each independently —O— or —CH₂—.

69. The compound of claim 68, wherein A₅ is —O—.

70. The compound of claim 68, wherein A₅ is —CH₂—.

71. The compound of any one of claims 68-70, wherein A₆ is —O—.

72. The compound of any one of claims 68-70, wherein A₆ is —CH₂—.

73. The compound of any one of claims 1-67, wherein L is 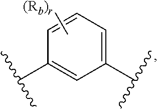
wherein R_{b} is halogen, alkyl, or alkoxy; and r is 0, 1, or 2.

74. The compound of claim 73, wherein R_{b} is halogen.

75. The compound of claim 74, wherein the halogen is fluoride.

76. The compound of any one of claims 73-75, wherein r is 1.

77. The compound of any one of claims 73-75, wherein r is 0.

78. The compound of any one of claims 1-67, wherein L is

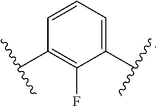 [SMILES: *c1cccc(*)c1F]

79. The compound of any one of claims 1-67, wherein L is a 5- or 6-membered heteroaryl linker having 1-2 nitrogen atoms.

80. The compound of any one of claims 1-67 and 79, wherein L is 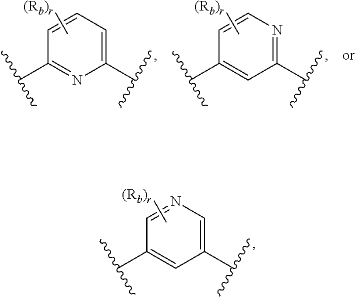
wherein R_{b} is halogen, alkyl, or alkoxy; and r is 0 or 1.

81. The compound of any one of claims 1-72, having the structure: 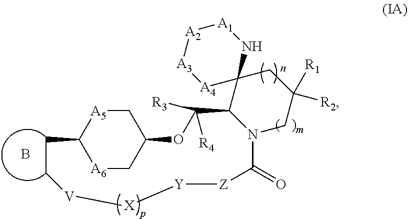
or a pharmaceutically acceptable salt or stereoisomer thereof.

82. The compound of any one of claims 1-67 and 73-80, having the structure: 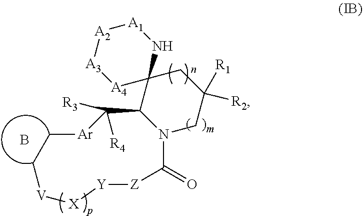
or a pharmaceutically acceptable salt or stereoisomer thereof,
wherein Ar is an aryl or heteroaryl linker.

83. The compound of claim 1, wherein the compound is selected from the group consisting of:

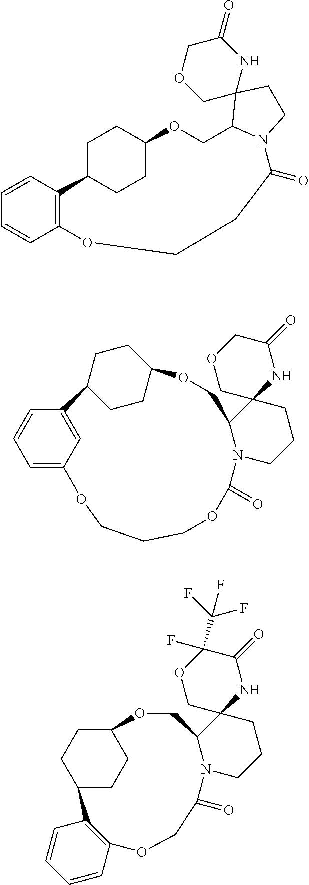 [SMILES: O=C1COCC2(CCN3C(=O)CCOc4ccccc4[C@H]4CC[C@H](CC4)OCC32)N1 ; O=C1COC[C@@]2(CCCN3C(=O)OCCCOc4cccc(c4)[C@H]4CC[C@H](CC4)OC[C@H]32)N1 ; O=C1COc2ccccc2[C@H]2CC[C@H](CC2)OC[C@@H]2N1CCC[C@@]21CO[C@](F)(C(F)(F)F)C(=O)N1]

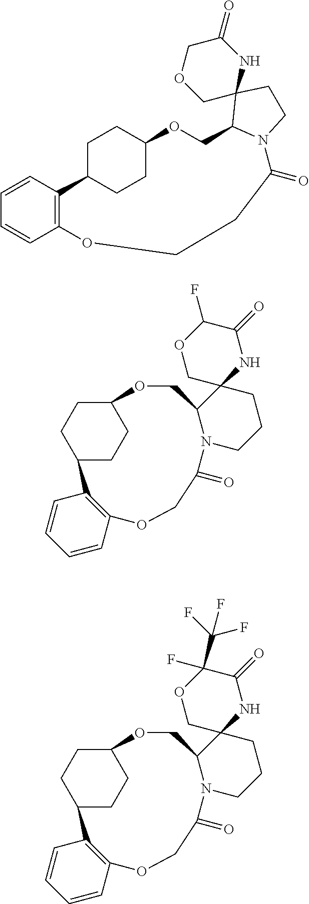

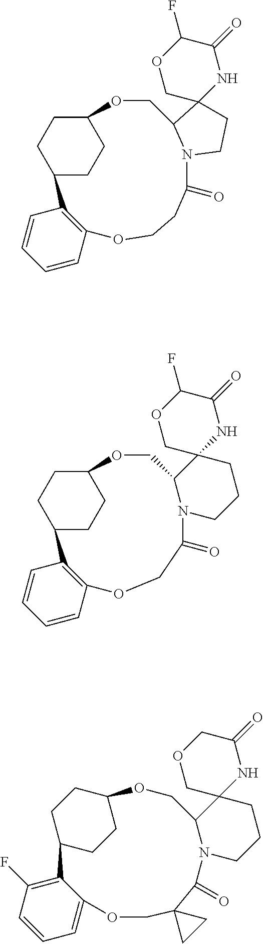

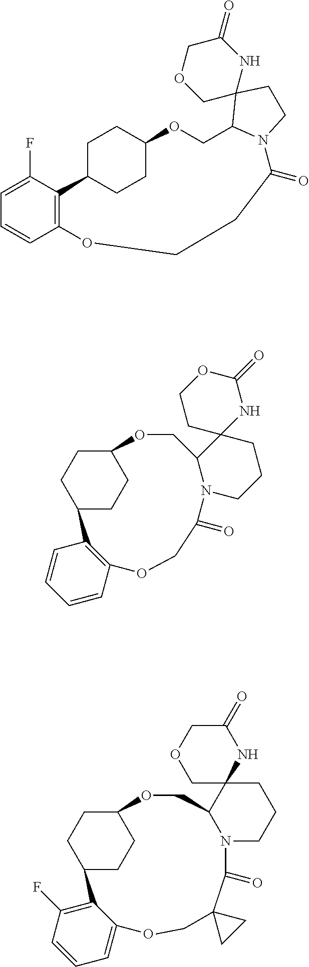 [SMILES: O=C1COCC2(CCN3C(=O)CCOc4cccc(F)c4[C@H]4CC[C@H](CC4)OCC32)N1 ; O=C1NC2(CCCN3C(=O)COc4ccccc4[C@H]4CC[C@H](CC4)OCC32)CCO1 ; O=C1COC[C@@]2(CCCN3C(=O)C4(CC4)COc4cccc(F)c4[C@H]4CC[C@H](CC4)OC[C@H]32)N1]

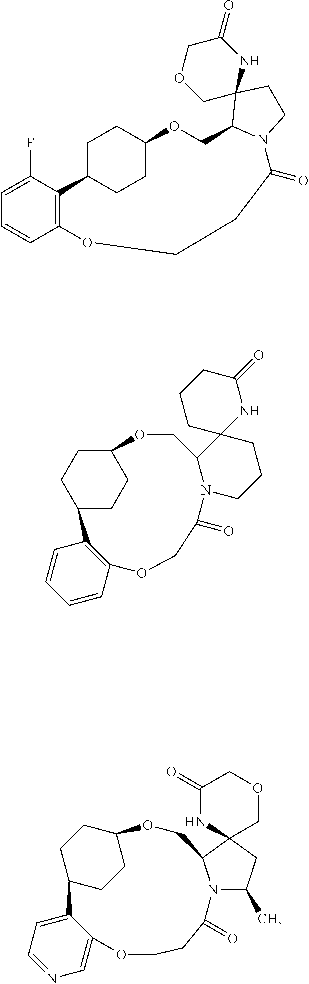

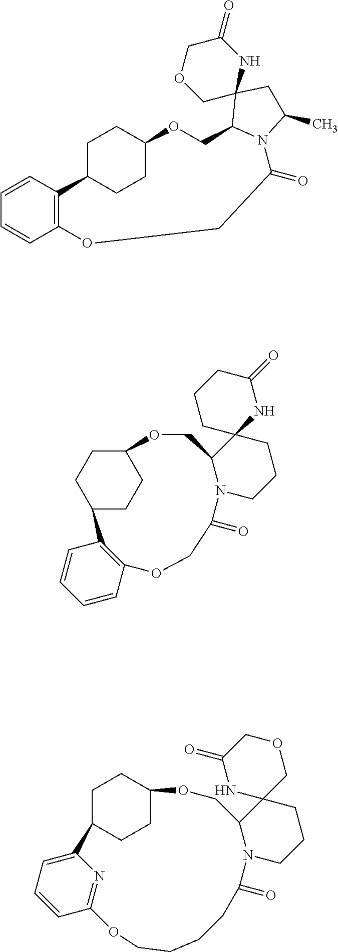 [SMILES: C[C@@H]1C[C@@]2(COCC(=O)N2)[C@@H]2CO[C@H]3CC[C@H](CC3)c3ccccc3OCC(=O)N12 ; O=C1CCC[C@@]2(CCCN3C(=O)COc4ccccc4[C@H]4CC[C@H](CC4)OC[C@H]32)N1 ; O=C1COCC2(CCCN3C(=O)CCCCOc4cccc(n4)[C@H]4CC[C@H](CC4)OCC32)N1]

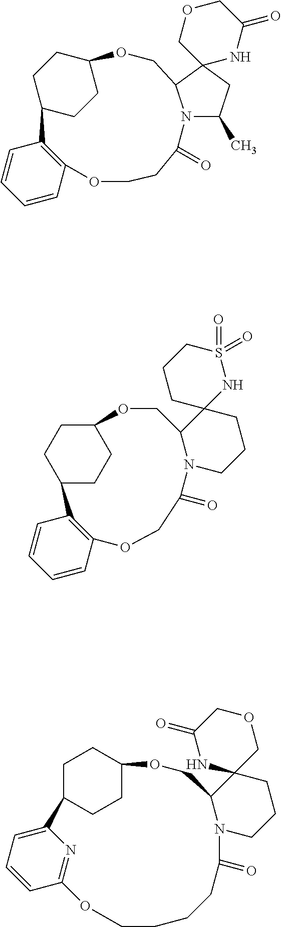 [SMILES: C[C@@H]1CC2(COCC(=O)N2)C2CO[C@H]3CC[C@H](CC3)c3ccccc3OCCC(=O)N21 ; O=C1COc2ccccc2[C@H]2CC[C@H](CC2)OCC2N1CCCC21CCCS(=O)(=O)N1 ; O=C1COC[C@@]2(CCCN3C(=O)CCCCOc4cccc(n4)[C@H]4CC[C@H](CC4)OC[C@H]32)N1]

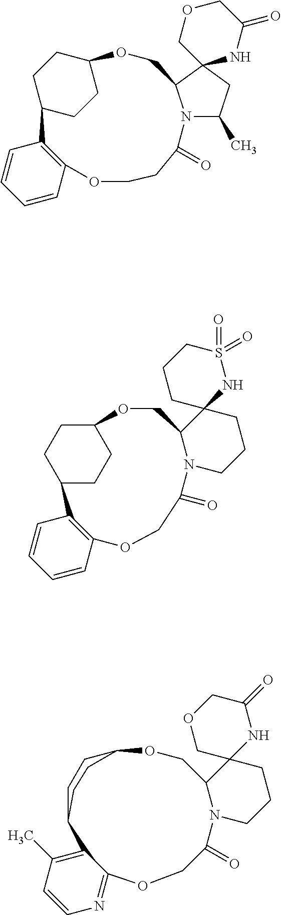 [SMILES: C[C@@H]1C[C@@]2(COCC(=O)N2)[C@@H]2CO[C@H]3CC[C@H](CC3)c3ccccc3OCCC(=O)N12 ; O=C1COc2ccccc2[C@H]2CC[C@H](CC2)OC[C@@H]2N1CCC[C@@]21CCCS(=O)(=O)N1 ; Cc1ccnc2c1[C@H]1CC[C@H](CC1)OCC1N(CCCC13COCC(=O)N3)C(=O)CO2]

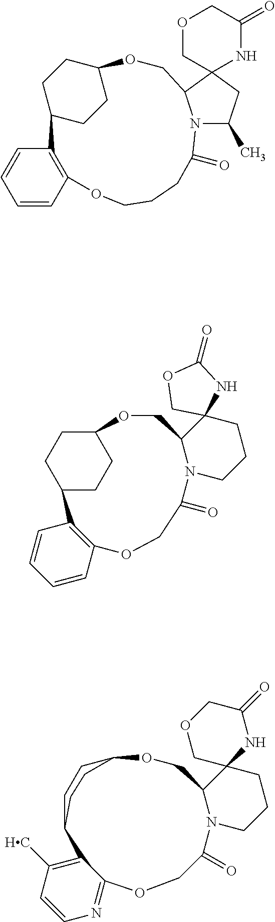

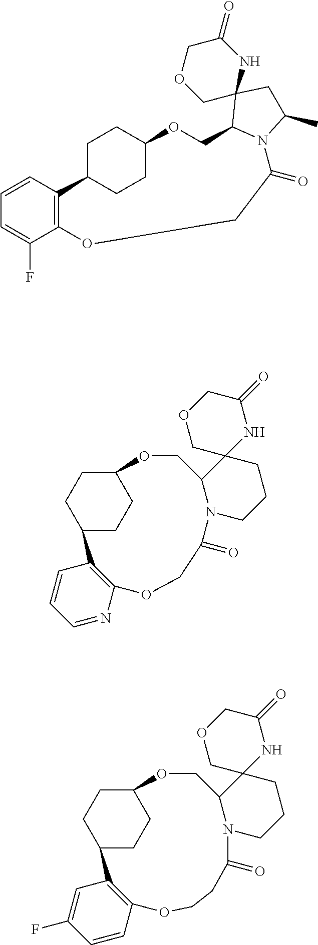 [SMILES: C[C@@H]1C[C@@]2(COCC(=O)N2)[C@@H]2CO[C@H]3CC[C@H](CC3)c3cccc(F)c3OCC(=O)N12 ; O=C1COCC2(CCCN3C(=O)COc4ncccc4[C@H]4CC[C@H](CC4)OCC32)N1 ; O=C1COCC2(CCCN3C(=O)CCOc4ccc(F)cc4[C@H]4CC[C@H](CC4)OCC32)N1]

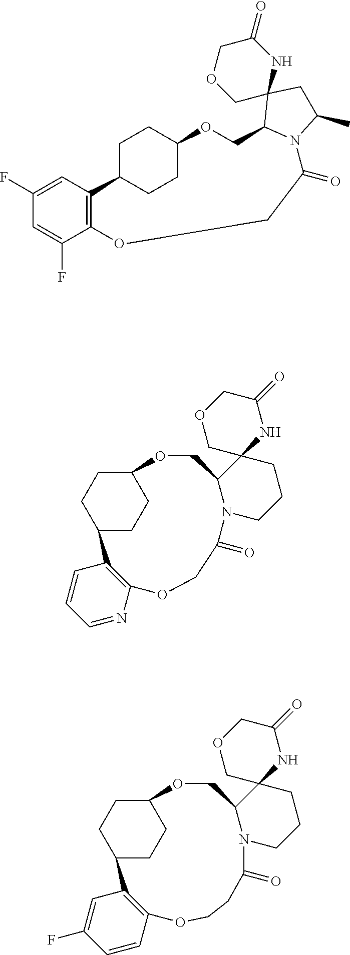 [SMILES: C[C@@H]1C[C@@]2(COCC(=O)N2)[C@@H]2CO[C@H]3CC[C@H](CC3)c3cc(F)cc(F)c3OCC(=O)N12 ; O=C1COC[C@@]2(CCCN3C(=O)COc4ncccc4[C@H]4CC[C@H](CC4)OC[C@H]32)N1 ; O=C1COC[C@@]2(CCCN3C(=O)CCOc4ccc(F)cc4[C@H]4CC[C@H](CC4)OC[C@H]32)N1]

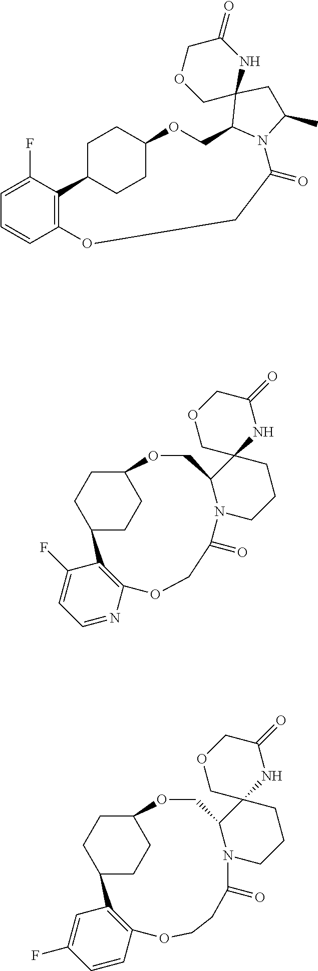 [SMILES: C[C@@H]1C[C@@]2(COCC(=O)N2)[C@@H]2CO[C@H]3CC[C@H](CC3)c3c(F)cccc3OCC(=O)N12 ; O=C1COC[C@@]2(CCCN3C(=O)COc4nccc(F)c4[C@H]4CC[C@H](CC4)OC[C@H]32)N1 ; O=C1COC[C@]2(CCCN3C(=O)CCOc4ccc(F)cc4[C@H]4CC[C@H](CC4)OC[C@@H]32)N1]

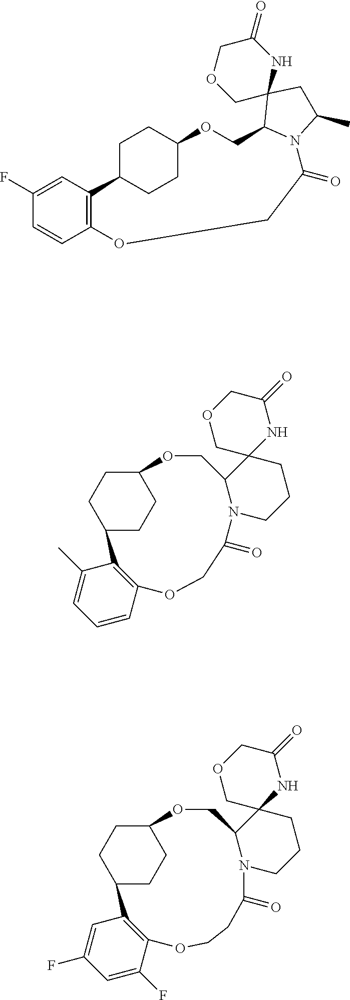

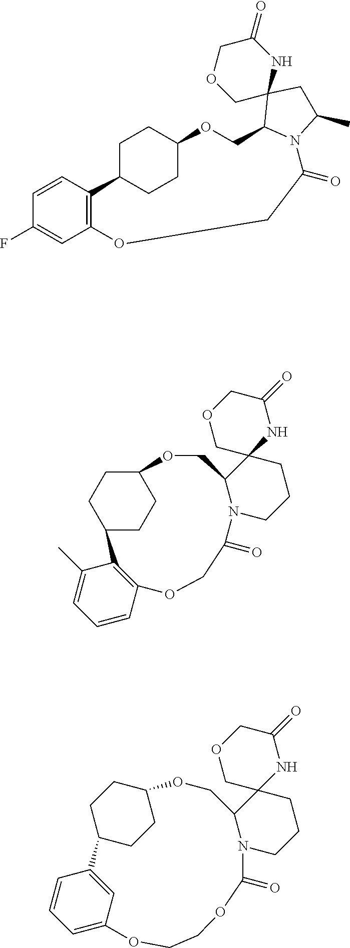

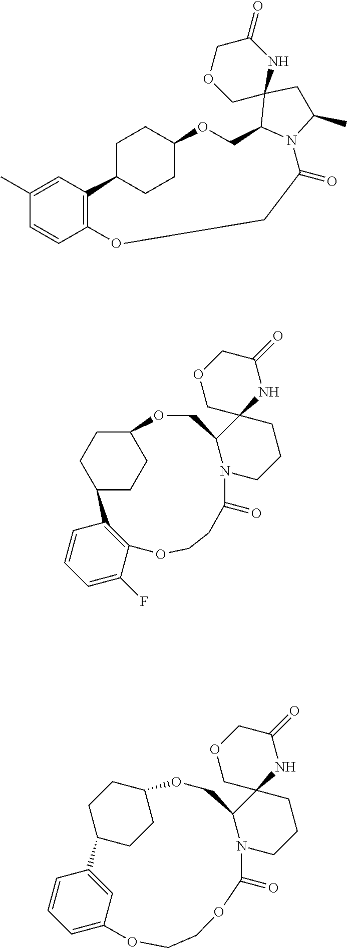

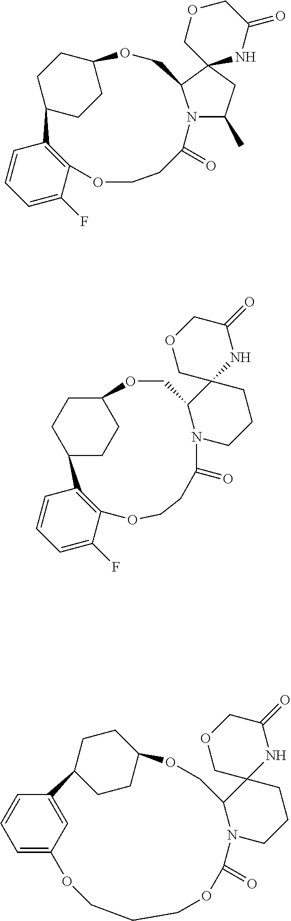 [SMILES: C[C@@H]1C[C@@]2(COCC(=O)N2)[C@@H]2CO[C@H]3CC[C@H](CC3)c3cccc(F)c3OCCC(=O)N12 ; O=C1COC[C@]2(CCCN3C(=O)CCOc4c(F)cccc4[C@H]4CC[C@H](CC4)OC[C@@H]32)N1 ; O=C1COCC2(CCCN3C(=O)OCCCOc4cccc(c4)[C@H]4CC[C@H](CC4)OCC32)N1]

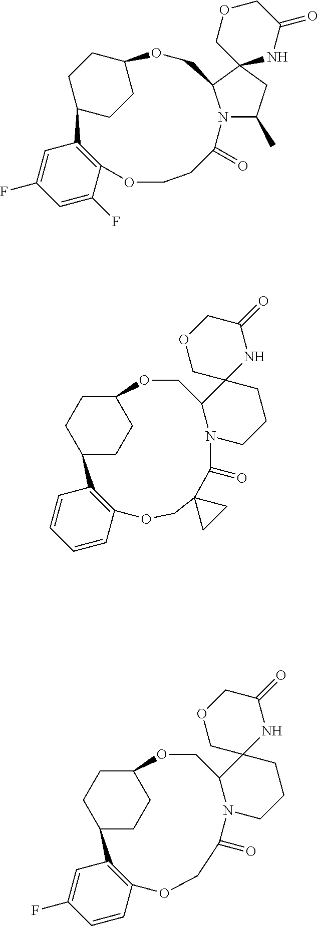 [SMILES: C[C@@H]1C[C@@]2(COCC(=O)N2)[C@@H]2CO[C@H]3CC[C@H](CC3)c3cc(F)cc(F)c3OCCC(=O)N12 ; O=C1COCC2(CCCN3C(=O)C4(CC4)COc4ccccc4[C@H]4CC[C@H](CC4)OCC32)N1 ; O=C1COCC2(CCCN3C(=O)COc4ccc(F)cc4[C@H]4CC[C@H](CC4)OCC32)N1]

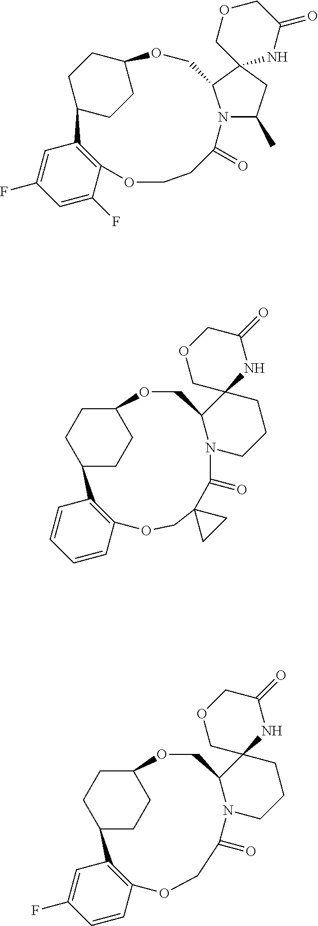 [SMILES: C[C@@H]1C[C@]2(COCC(=O)N2)[C@H]2CO[C@H]3CC[C@H](CC3)c3cc(F)cc(F)c3OCCC(=O)N12 ; O=C1COC[C@@]2(CCCN3C(=O)C4(CC4)COc4ccccc4[C@H]4CC[C@H](CC4)OC[C@H]32)N1 ; O=C1COC[C@@]2(CCCN3C(=O)COc4ccc(F)cc4[C@H]4CC[C@H](CC4)OC[C@H]32)N1]

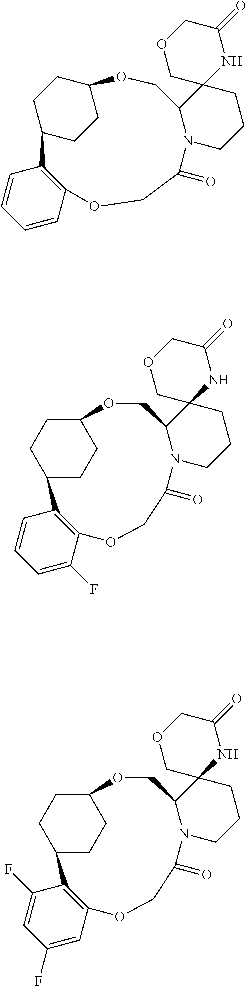 [SMILES: O=C1COCC2(CCCN3C(=O)COc4ccccc4[C@H]4CC[C@H](CC4)OCC32)N1 ; O=C1COC[C@@]2(CCCN3C(=O)COc4c(F)cccc4[C@H]4CC[C@H](CC4)OC[C@H]32)N1 ; O=C1COC[C@@]2(CCCN3C(=O)COc4cc(F)cc(F)c4[C@H]4CC[C@H](CC4)OC[C@H]32)N1]

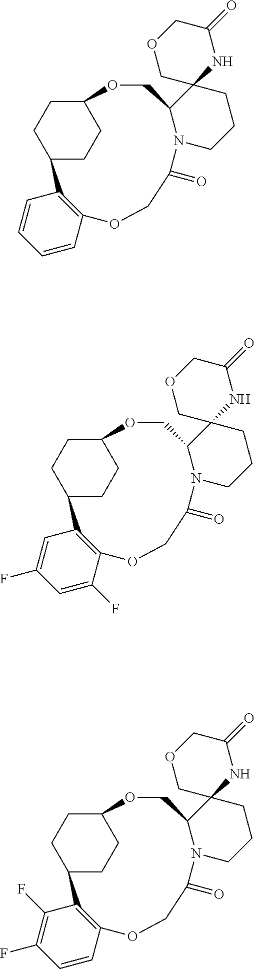 [SMILES: O=C1COC[C@@]2(CCCN3C(=O)COc4ccccc4[C@H]4CC[C@H](CC4)OC[C@H]32)N1 ; O=C1COC[C@]2(CCCN3C(=O)COc4c(F)cc(F)cc4[C@H]4CC[C@H](CC4)OC[C@@H]32)N1 ; O=C1COC[C@@]2(CCCN3C(=O)COc4ccc(F)c(F)c4[C@H]4CC[C@H](CC4)OC[C@H]32)N1]

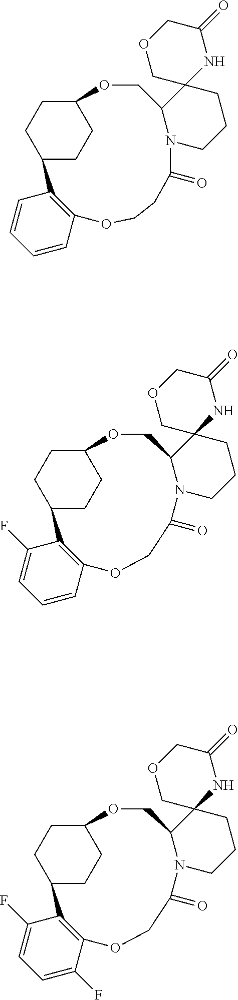 [SMILES: O=C1COCC2(CCCN3C(=O)CCOc4ccccc4[C@H]4CC[C@H](CC4)OCC32)N1 ; O=C1COC[C@@]2(CCCN3C(=O)COc4cccc(F)c4[C@H]4CC[C@H](CC4)OC[C@H]32)N1 ; O=C1COC[C@@]2(CCCN3C(=O)COc4c(F)ccc(F)c4[C@H]4CC[C@H](CC4)OC[C@H]32)N1]

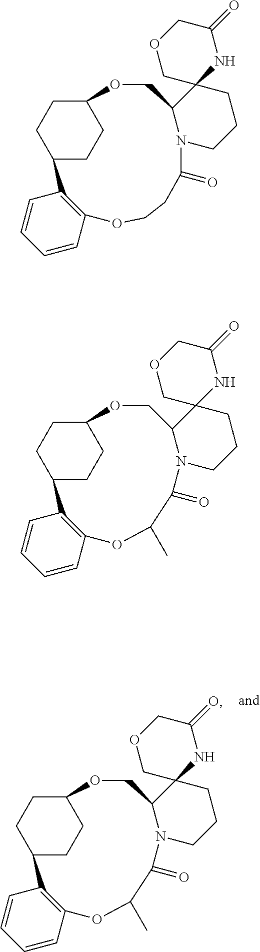 [SMILES: O=C1COC[C@@]2(CCCN3C(=O)CCOc4ccccc4[C@H]4CC[C@H](CC4)OC[C@H]32)N1 ; CC1Oc2ccccc2[C@H]2CC[C@H](CC2)OCC2N(CCCC23COCC(=O)N3)C1=O ; CC1Oc2ccccc2[C@H]2CC[C@H](CC2)OC[C@@H]2N(CCC[C@@]23COCC(=O)N3)C1=O]

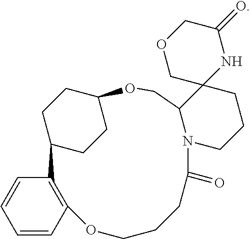 [SMILES: O=C1COCC2(CCCN3C(=O)CCCOc4ccccc4[C@H]4CC[C@H](CC4)OCC32)N1]

84. A pharmaceutical composition comprising a compound of any one of claims 1-83 or a pharmaceutically acceptable salt thereof, and a pharmaceutically acceptable excipient.

85. A method of treating a disease or disorder that is treatable by administration of an orexin agonist, the method comprising administering to a subject in need thereof a therapeutically effective amount of a compound of any one of claims 1-83.

86. A method of treating a disease or disorder by modulating one or more orexin receptors, the method comprising administering to a subject in need thereof a therapeutically effective amount of a compound of any one of claims 1-83.

87. A method of treating, preventing, ameliorating, controlling or reducing the risk of a disease or disorder associated with one or more orexin receptors, the method comprising administering to a subject in need thereof a therapeutically effective amount of a compound of any one of claims 1-83.
